(19) United States
(12) Patent Application Publication
(10) Pub. No.: US 2024/0262832 A1
(43) Pub. Date: Aug. 8, 2024

(54) SMALL MOLECULE MODULATORS OF GLUCOCEREBROSIDASE ACTIVITY AND USES THEREOF

(71) Applicant: Vanqua Bio, Inc., Chicago, IL (US)

(72) Inventors: Kevin Hunt, Chicago, IL (US); Jianbin Zheng, Glencoe, IL (US); Sida Shen, Chicago, IL (US)

(73) Assignee: Vanqua Bio, Inc., Chicago, IL (US)

(21) Appl. No.: 18/288,928

(22) PCT Filed: Apr. 28, 2022
(86) PCT No.: PCT/US2022/026715

§ 371 Date: Oct. 30, 2023

Related U.S. Application Data
(60) Provisional application No. 63/182,728, filed on Apr. 30, 2021.

(51) Int. Cl. C07D 487/04; A61K 31/454; A61K 31/4545; A61K 31/46; A61K 31/497; A61K 31/4985; A61K 31/501; A61K 31/506; A61K 31/519; A61K 31/5377; C07D 211/52; C07D 401/04; C07D 401/06; C07D 401/14; C07D 405/06; C07D 405/14; C07D 413/06; C07D 413/12; C07D 413/14; C07D 417/06; C07D 417/14; C07D 471/04

(52) U.S. Cl. CPC C07D 487/04; A61K 31/454; A61K 31/4545; A61K 31/46; A61K 31/497; A61K 31/4985; A61K 31/501; A61K 31/506; A61K 31/519; A61K 31/5377; C07D 211/52; C07D 401/04; C07D 401/06; C07D 401/14; C07D 405/06; C07D 405/14; C07D 413/06; C07D 413/12; C07D 413/14; C07D 417/06; C07D 417/14; C07D 471/04

(57) ABSTRACT

Provided herein are compounds that modulate glucocerebrosidase (GCase), an enzyme whose activity is associated with neurological diseases and disorders (e.g., Gaucher's disease. Parkinson's disease). Also provided are pharmaceutical compositions and kits comprising the compounds, and methods of treating GCase-related diseases and disorders (e.g., Gaucher's disease. Parkinson's disease) with the compounds in a subject, by administering the compounds and/or compositions described herein.

### RELATED APPLICATION

This application claims priority under 35 U.S.C. § 119(e) to U.S. Provisional Application 63/182,728, filed Apr. 30, 2021, the entirety of which is incorporated herein by reference.

### BACKGROUND

Glucocerebrosidase (EC 3.2.1.45), also called β-glucocerebrosidase, β-glucosidase, D-glucosyl-N-acylsphingosine glucohydrolase, or GCase, is an enzyme having glucosylceramidase activity. Glucocerebrosidase is required to cleave the beta-glucosidic linkage of the chemical glucocerebroside, which is an intermediate in glycolipid metabolism. Glucocerebrosidase is localized in the lysosome and disabling mutations in the gene for glucocerebrosidase (GBA1) are associated with abnormal accumulation of lipids in lysosomes.

Genetic diseases caused by mutations in GBA1 include neurodegenerative diseases such as Gaucher's disease and Parkinson's disease. Current treatments for diseases such Type 1 Gaucher's disease are limited to enzyme replacement therapy (ERT) administered every two weeks. ERT is very expensive and not effective for neuronopathic forms of Gaucher's disease. Efforts to discover and employ small molecule compounds to activate Gcase have been met with limited success. Thus, there is a need for new compounds that effectively activate Gcase and are useful in the treatment of neurodegenerative diseases (e.g., Gaucher's disease and Parkinson's disease).

### SUMMARY

The present disclosure provides compounds that are modulators of GCase. These compounds provide new compositions and methods for the treatment of diseases associated with GCase activity (e.g., neurodegenerative diseases, such as Gaucher's disease and Parkinson's disease).

In one aspect, provided are compounds of Formula (I):

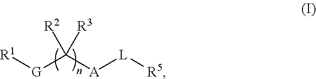

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, substituted or unsubstituted aryl, substituted or unsubstituted carbocyclyl, substituted or unsubstituted heterocyclyl, pentyl, butyl, methyl, —CH₂CH₂CH(CH₃)₂, or hydrogen, or optionally a heterocyclyl forming a spirocyclic ring system with A when n is 0 and G is a bond;
G is a bond, —S(O)₂—, —NR²—, —CH₂CH₂O—, —CH₂O—, —O— or —CR²R³—;
R² and R³ are each independently hydrogen, halogen, or substituted or unsubstituted alkyl, or R² and R³ on the same carbon form with that carbon a carbonyl;
n is 1 or 0;
A is

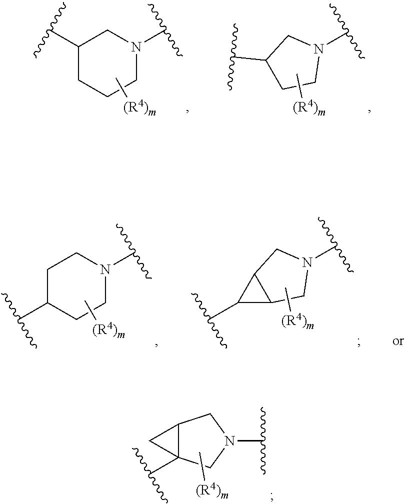

each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, —C(═O)CF₂—, —C(═O)CH(Ph)—, —C(═O)CH(iPr)—, —C(═O)CH(Et)—, —C(═O)CH(Me)—, —C(═O)C(CH₃)₂—, —C(═O)CH(OMe)—, —C(═O)CH₂CH₂—, —C(═O)CH₂CH₂CH₂—, —C(═O)CH₂CH₂CH₂O—, —C(═O)CH(CH₃)CH₂—, —C(═O)CH₂O—, —C(═O)CH₂OCH₂—, —C(═O)CH(CH₃)O—, —C(═O)CH₂CH═CH—, —C(═O)NHCH₂CH₂CH₂—, —C(═O)NHCH₂CH₂—, —CH₂—, —CH₂CH₂CH₂—, —CH₂C(CH₃)₂—, —C(═O)NH—, or —CH₂C(═O)NH—; and
R⁵ is substituted or unsubstituted heteroaryl, substituted or unsubstituted heterocyclyl, substituted or unsubstituted heteroarylalkyl, substituted or unsubstituted carbocyclyl, substituted or unsubstituted aryl, methyl, ethyl, butyl, pentyl, t-butyl, —CH₂CH₂CH(CH₃)₂, —SCF₃, or —OCH₂CH(CH₃)₂.

In another aspect, provided are compounds of Formula (I):

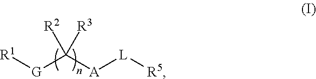

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, substituted or unsubstituted aryl, substituted or unsubstituted carbocyclyl, or substituted or unsubstituted heterocyclyl, pentyl, butyl, or —CH₂CH₂CH(CH₃)₂;
G is —S(O)₂—, —NR²—, —CH₂CH₂O—, —CH₂O—, —O— or —CR²R³—;
R² and R³ are each independently hydrogen, halogen, or substituted or unsubstituted alkyl;
n is 1 or 0;
A is

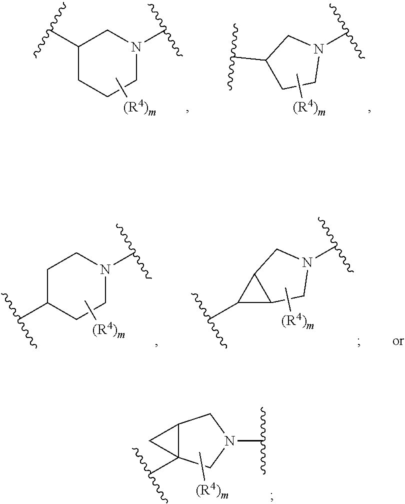

each R⁴ is independently halogen, substituted or unsubstituted alkyl, substituted or unsubstituted alkoxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, or —C(═O)CH₂O—; and
R⁵ is substituted or unsubstituted heteroaryl, substituted or unsubstituted heterocyclyl, substituted or unsubstituted heteroarylalkyl, substituted or unsubstituted aryl, substituted or unsubstituted carbocyclyl, or substituted or unsubstituted aryloxyalkyl.

In another aspect, provided are compounds of Formula (I):

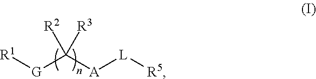

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

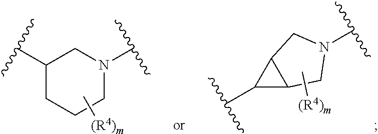

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted indolyl, substituted or unsubstituted oxadiazolyl, substituted or unsubstituted triazolyl, or substituted or unsubstituted pyrazinyl.

In another aspect, provided are compounds of Formula (I):

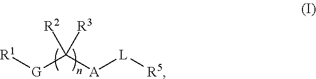

and pharmaceutically acceptable salts, co-crystals, tautomers, stereoisomers, solvates, hydrates, polymorphs, isotopically enriched derivatives, or prodrugs thereof, wherein:
R¹ is substituted or unsubstituted heteroaryl, or substituted or unsubstituted aryl;
G is —O— or —CR²R³—;
R² and R³ are each independently hydrogen, halogen, or substituted or unsubstituted alkyl;
n is 1 or 0;
A is

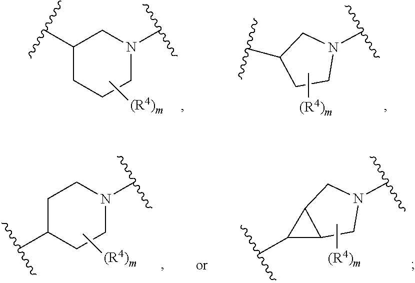

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted heteroaryl, substituted or unsubstituted heterocyclyl, substituted or unsubstituted heteroarylalkyl, substituted or unsubstituted carbocyclyl, or substituted or unsubstituted aryloxyalkyl.

In certain embodiments of Formula (I):
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

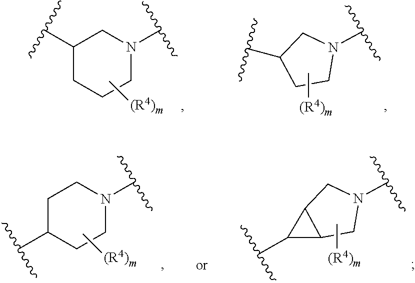

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted heteroarylalkyl, substituted or unsubstituted cycloalkyl, or substituted or unsubstituted aryloxyalkyl.

In certain embodiments, the compounds of Formula (I) are compounds of Formula (I-a), (I-b), (I-c), (I-d), (I-e), (I-f), (I-g), (I-h), (I-i), (II-a), (II-b), (II-c), (II-d), (III-a), (III-b), (III-c), (III-d), (IV-a), (IV-b), (IV-c), (IV-d), (IV-e), (V-a), (V-b), (V-c), or (V-d):

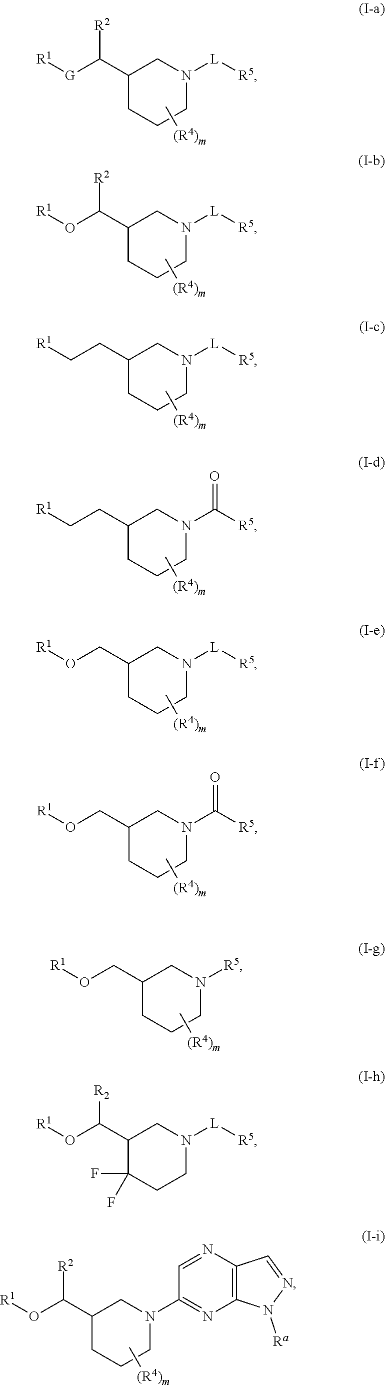

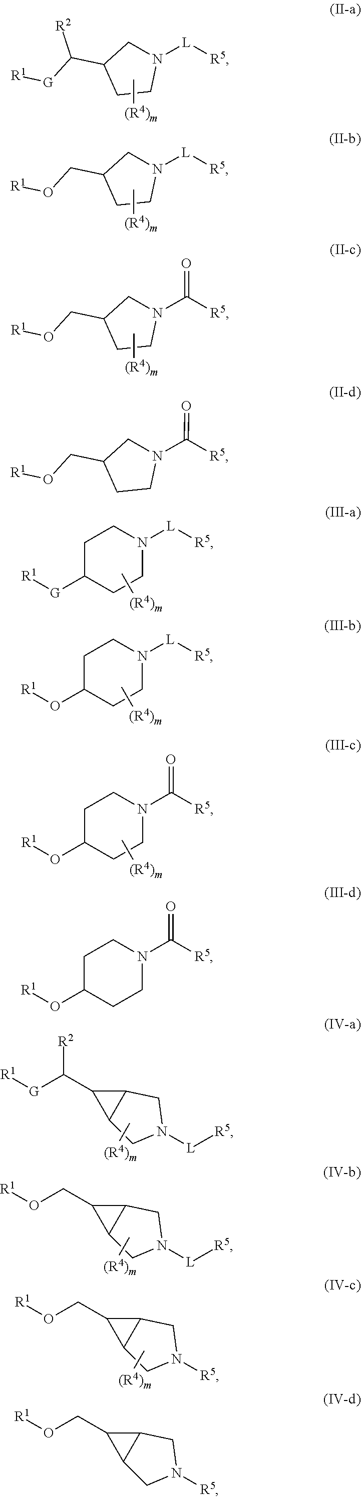

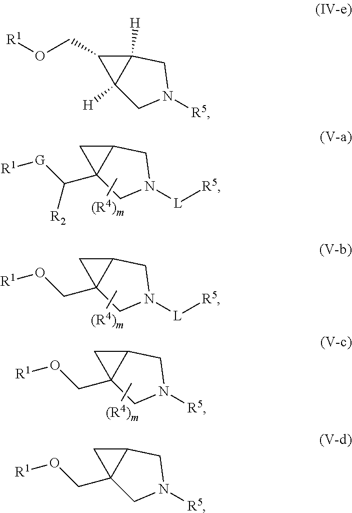

or pharmaceutically acceptable salts thereof.

In another aspect, provided are pharmaceutical compositions comprising a compound of Formula (I), or a pharmaceutically acceptable salt thereof, and optionally a pharmaceutically acceptable excipient.

In another aspect, provided are methods of treating a disease or disorder in a subject in need thereof, the method comprising administering a compound of Formula (I), or a pharmaceutically acceptable salt thereof, or a pharmaceutical composition comprising a compound of Formula (I) to the subject.

In certain embodiments, the disease or disorder is associated with glucocerebrosidase activity. In certain embodiments, the disease or disorder is a neurological disease or disorder. In certain embodiments, the neurological disease or disorder is Parkinson's disease or Gaucher's disease.

In another aspect, provided are methods of activating glucocerebrosidase, the method comprising contacting glucocerebrosidase with an effective amount of a compound of Formula (I), or a pharmaceutically acceptable salt thereof, or a pharmaceutical composition comprising a compound of Formula (I) to the subject.

In another aspect, provided are kits comprising a compound of Formula (I), or a pharmaceutically acceptable salt thereof, or a pharmaceutical composition comprising a compound of Formula (I), or a pharmaceutically acceptable salt thereof. In certain embodiments, the kits further comprise instructions for administration (e.g., human administration).

The details of certain embodiments of the invention are set forth in the Detailed Description of Certain Embodiments, as described below. Other features, objects, and advantages of the invention will be apparent from the Definitions, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stercoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (iPr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₂), pentadienyl (C₃), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

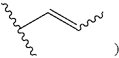

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1.4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Arylalkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroarylalkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

When substituted, exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X″ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl. C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R″ groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)₃⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{cc})₃, —OP(R^{cc})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R)₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumcoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylidencamine, N-diphenylmethylencamine, N-[(2-pyridyl)mesityl]methyleneamine, N—(N′,N′-dimethylaminomethylene)amine, N,N′-isopropylidenediamine, N-p-nitrobenzylideneamine, N-salicylidencamine, N-5-chlorosalicylideneamine, N-(5-chloro-2-hydroxyphenyl)phenylmethylencamine, N-cyclohexylidencamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfonamide, o-nitrobenzenesulfenamide (Nps), 2,4-dinitrobenzenesulfenamide, pentachlorobenzenesulfonamide, 2-nitro-4-methoxybenzenesulfonamide, triphenylmethylsulfonamide, and 3-nitropyridinesulfonamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, and Claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stercoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

The term “modulate” means decreasing or inhibiting activity and/or increasing or augmenting activity. For example, modulating glucocerebrosidase activity means decreasing or inhibiting glucocerebrosidase activity and/or increasing or augmenting glucocerebrosidase activity. The compounds disclosed herein may be administered to modulate glucocerebrosidase activity for example, as a chaperone or activator.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering.” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, or inhibiting the progress of a disease described herein. In some embodiments, treatment may be administered after one or more signs or symptoms of the disease have developed or have been observed. Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for GCase activation (e.g., at least 5%, at least 10%, at least 20%, at least 30%, at least 40%, at least 50%, at least 60%, at least 70%, at least 80%, at least 90%, at least 95%, at least 100%, at least 150%, at least 200%, at least 250%, at least 300%, or at least 500% increase in the enzymatic activity of GCase). In certain embodiments, a therapeutically effective amount is an amount sufficient for treating a disease or disorder (e.g., neurological disorder). In certain embodiments, a therapeutically effective amount is an amount sufficient for GCase activation and treating a disease or disorder (e.g., neurological disorder).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for GCase activation. In certain embodiments, a prophylactically effective amount is an amount sufficient for treating a disease or disorder (e.g., neurological disorder). In certain embodiments, a prophylactically effective amount is an amount sufficient for GCase activation and treating a disease or disorder (e.g., neurological disorder).

As used herein, the term “activate” or “activation” in the context of enzymes, for example, in the context of GCase, refers to an increase in the activity of the enzyme. In some embodiments, the term refers to an increase of the level of enzyme activity, e.g., GCase activity, to a level that is statistically significantly higher than an initial level, which may, for example, be a baseline level of enzyme activity (e.g., of wild-type GCase). In some embodiments, the term refers to an increase in the level of enzyme activity, e.g., GCase activity, to a level that is greater than 1%, greater than 5%, greater than 10%, greater than 25%, greater than 50%, greater than 75%, greater than 100%, greater than 150%, greater than 200%, greater than 300%, greater than 400%, greater than 500%, or greater than 1000% of an initial level, which may, for example, be a baseline level of enzyme activity.

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

### DETAILED DESCRIPTION OF CERTAIN EMBODIMENTS

Provided herein are compounds that are modulators of GCase (e.g., GCase activators). In one aspect, the provided GCase modulators are compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with GCase activity (e.g., neurological diseases and disorders) in a subject in need thereof.

The compounds described herein interact with GCase. As described herein, the therapeutic effect may be a result of modulation (e.g., activation), binding, and/or modification of GCase by the compounds described herein. The compounds may be provided for use in any composition, kit, or method described herein as a pharmaceutically acceptable salt, co-crystal, tautomer, stereoisomer, solvate, hydrate, polymorph, isotopically enriched derivative, or prodrug thereof.

### Compounds of Formula (I)

In one aspect, disclosed is a compound of Formula (I):

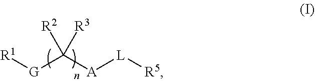

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, substituted or unsubstituted aryl, substituted or unsubstituted carbocyclyl, substituted or unsubstituted heterocyclyl, pentyl, butyl, methyl, —CH₂CH₂CH(CH₃)₂, or hydrogen, or optionally a heterocyclyl forming a spirocyclic ring system with A when n is 0 and G is a bond;
G is a bond, —S(O)₂—, —NR²—, —CH₂CH₂O—, —CH₂O—, —O— or —CR²R³—;
R² and R³ are each independently hydrogen, halogen, or substituted or unsubstituted alkyl, or R² and R³ on the same carbon form with that carbon a carbonyl;
n is 1 or 0;
A is

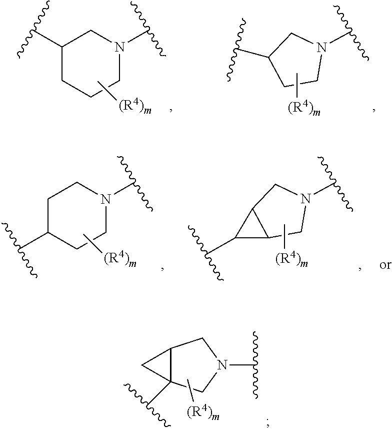

each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, —C(═O)CF₂—, —C(═O)CH(Ph)—, —C(═O)CH(iPr)—, —C(═O)CH(Et)—, —C(═O)CH(Me)—, —C(═O)C(CH₃)₂—, —C(═O)CH(OMe)—, —C(═O)CH₂CH₂—, —C(═O)CH₂CH₂CH₂—, —C(═O)CH₂CH₂CH₂O—, —C(═O)CH(CH₃)CH₂—, —C(═O)CH₂O—, —C(═O)CH₂OCH₂—, —C(═O)CH(CH₃)O—, —C(═O)CH₂CH═CH—, —C(═O)NHCH₂CH₂CH₂—, —C(═O)NHCH₂CH₂—, —CH₂—, —CH₂CH₂CH₂—, —CH₂C(CH₃)₂—, —C(═O)NH—, or —CH₂C(═O)NH—; and
R⁵ is substituted or unsubstituted heteroaryl, substituted or unsubstituted heterocyclyl, substituted or unsubstituted heteroarylalkyl, substituted or unsubstituted carbocyclyl, substituted or unsubstituted aryl, methyl, ethyl, butyl, pentyl, t-butyl, —CH₂CH₂CH(CH₃)₂, —SCF₃, or —OCH₂CH(CH₃)₂

In certain embodiments of the compound of Formula (I):

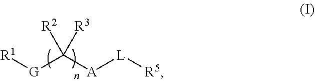

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, substituted or unsubstituted aryl, substituted or unsubstituted carbocyclyl, or substituted or unsubstituted heterocyclyl, pentyl, butyl, or
   —CH₂CH₂CH(CH₃)₂;
G is —S(O)₂—, —NR²—, —CH₂CH₂O—, —CH₂O—, —O— or —CR²R³—;
R² and R³ are each independently hydrogen, halogen, or substituted or unsubstituted alkyl;
n is 1 or 0;
A is

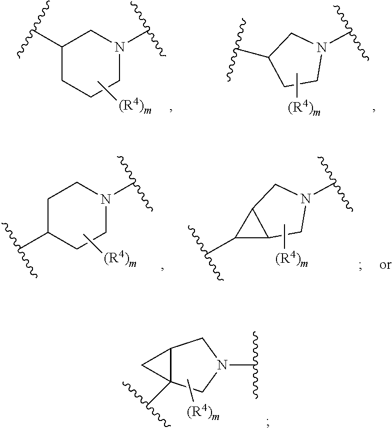

each R⁴ is independently halogen, substituted or unsubstituted alkyl, substituted or unsubstituted alkoxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, or —C(═O)CH₂O—; and
R⁵ is substituted or unsubstituted heteroaryl, substituted or unsubstituted heterocyclyl, substituted or unsubstituted heteroarylalkyl, substituted or unsubstituted aryl, substituted or unsubstituted carbocyclyl, or substituted or unsubstituted aryloxyalkyl.

In certain embodiments of the compound of Formula (I):

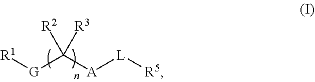

or a pharmaceutically acceptable salt, co-crystal, tautomer, stereoisomer, solvate, hydrate, polymorph, isotopically enriched derivative, or prodrug thereof, wherein:
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

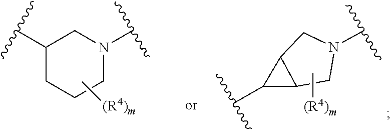

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted indolyl, substituted or unsubstituted oxadiazolyl, substituted or unsubstituted triazolyl, or substituted or unsubstituted pyrazinyl.

In certain embodiments of the compound of Formula (I):

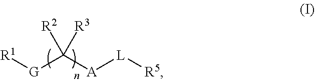

or a pharmaceutically acceptable salt, co-crystal, tautomer, stereoisomer, solvate, hydrate, polymorph, isotopically enriched derivative, or prodrug thereof, wherein:
R¹ is substituted or unsubstituted heteroaryl, or substituted or unsubstituted aryl;
G is —O— or —CR²R³—;
R² and R³ are each independently hydrogen, halogen, or substituted or unsubstituted alkyl;
n is 1 or 0;
A is

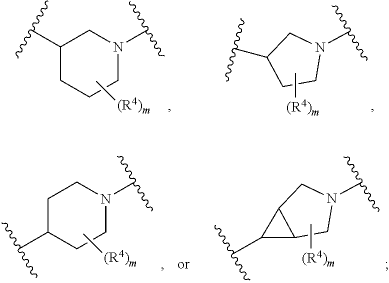

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted heteroaryl, substituted or unsubstituted heterocyclyl, substituted or unsubstituted heteroarylalkyl, substituted or unsubstituted carbocyclyl, or substituted or unsubstituted aryloxyalkyl.

In certain embodiments of the compound of Formula (I):
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

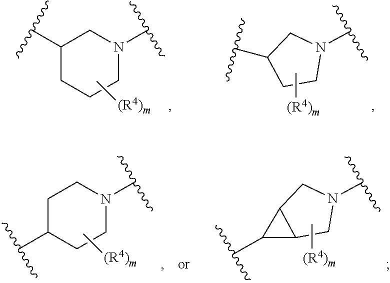

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted heteroarylalkyl, substituted or unsubstituted cycloalkyl, or substituted or unsubstituted aryloxyalkyl.

### R¹

As described herein, R¹ is substituted or unsubstituted heteroaryl, substituted or unsubstituted aryl, substituted or unsubstituted carbocyclyl, substituted or unsubstituted heterocyclyl, pentyl, butyl, methyl, —CH₂CH₂CH(CH₃)₂, or hydrogen, or optionally a heterocyclyl forming a spirocyclic ring system with A when n is 0 and G is a bond.

In certain embodiments, R¹ is substituted or unsubstituted heteroaryl, substituted or unsubstituted aryl, substituted or unsubstituted carbocyclyl, or substituted or unsubstituted heterocyclyl, pentyl, butyl, or —CH₂CH₂CH(CH₃)₂.

In certain embodiments, R¹ is substituted or unsubstituted heteroaryl, or substituted or unsubstituted aryl. In certain embodiments, R¹ is substituted or unsubstituted heteroaryl, or substituted or unsubstituted phenyl. In certain embodiments, R¹ is substituted or unsubstituted pyridinyl, or substituted or unsubstituted aryl.

In certain embodiments, R¹ is substituted or unsubstituted pyrazinyl, substituted or unsubstituted pyridinyl, or substituted or unsubstituted phenyl. In certain embodiments, R¹ is substituted or unsubstituted pyridinyl, or substituted or unsubstituted phenyl. In certain embodiments, R¹ is substituted pyridinyl, or substituted or unsubstituted phenyl.

In certain embodiments, R¹ is pyridinyl substituted with haloalkyl or haloalkoxy, unsubstituted phenyl, or phenyl substituted with halogen, haloalkyl, or alkyl. In certain embodiments. R¹ is pyridinyl substituted with halogen, haloalkyl or haloalkoxy; unsubstituted phenyl; or phenyl substituted with halogen, haloalkyl, or alkyl. In certain embodiments, R¹ is pyridinyl substituted with halogen, C₁₋₄ haloalkyl or C₁₋₄ haloalkoxy; unsubstituted phenyl; or phenyl substituted with halogen, C₁₋₄ haloalkyl, or C₁₋₄ alkyl.

In certain embodiments, R¹ is pyridinyl substituted with fluoro, fluoroalkyl or fluoroalkoxy; unsubstituted phenyl; or phenyl substituted with fluoro, fluoroalkyl, or alkyl. In certain embodiments, R¹ is pyridinyl substituted with fluoro, C₁₋₄ fluoroalkyl or C₁₋₄ fluoroalkoxy; unsubstituted phenyl; or phenyl substituted with halogen, C₁₋₄ fluoroalkyl, or C₁₋₄ alkyl.

In certain embodiments, R¹ is pyridinyl substituted with haloalkyl or haloalkoxy; unsubstituted phenyl; or phenyl substituted with haloalkyl or alkyl. In certain embodiments, R¹ is pyridinyl substituted with C₁₋₄ haloalkyl or C₁₋₄ haloalkoxy; unsubstituted phenyl; or phenyl substituted with C₁₋₄ haloalkyl or C₁₋₄ alkyl.

In certain embodiments, R¹ is pyridinyl substituted with fluoroalkyl or fluoroalkoxy; unsubstituted phenyl; or phenyl substituted with fluoroalkyl or alkyl. In certain embodiments, R¹ is pyridinyl substituted with C₁₋₄ fluoroalkyl or C₁₋₄ fluoroalkoxy; unsubstituted phenyl; or phenyl substituted with C₁₋₄ fluoroalkyl or C₁₋₄ alkyl.

In certain embodiments, R¹ is pyridinyl substituted with haloalkyl or haloalkoxy. In certain embodiments, R¹ is pyridinyl substituted with haloalkyl. In certain embodiments, R¹ is pyridinyl substituted with C₁₋₄ haloalkyl or C₁₋₄ haloalkoxy. In certain embodiments, R¹ is pyridinyl substituted with C₁₋₄ haloalkyl.

In certain embodiments. R¹ is pyridinyl substituted with fluoroalkyl or fluoroalkoxy. In certain embodiments, R¹ is pyridinyl substituted with fluoroalkyl. In certain embodiments, R¹ is pyridinyl substituted with C₁₋₄ fluoroalkyl or C₁₋₄ fluoroalkoxy. In certain embodiments, R¹ is pyridinyl substituted with C₁₋₄ fluoroalkyl.

In certain embodiments, R¹ is pyridinyl substituted with haloalkoxy. In certain embodiments, R¹ is pyridinyl substituted with C₁₋₄ haloalkoxy.

In certain embodiments, R¹ is pyridinyl substituted with fluoroalkoxy. In certain embodiments, R¹ is pyridinyl substituted with C₁₋₄ fluoroalkoxy.

In certain embodiments, R¹ is unsubstituted phenyl. In certain embodiments, R¹ is phenyl substituted with halogen, haloalkyl, or alkyl. In certain embodiments, R¹ is phenyl substituted with haloalkyl or alkyl. In certain embodiments, R¹ is phenyl substituted with C₁₋₄ haloalkyl or C₁₋₄ alkyl. In certain embodiments, R¹ is phenyl substituted with fluoroalkyl or alkyl. In certain embodiments, R¹ is phenyl substituted with C₁₋₄ fluoroalkyl or C₁₋₄ alkyl.

In certain embodiments, R¹ is phenyl substituted with haloalkyl. In certain embodiments, R¹ is phenyl substituted with fluoroalkyl. In certain embodiments, R¹ is phenyl substituted with C₁₋₄ fluoroalkyl. In certain embodiments, R¹ is phenyl substituted with fluoroalkyl. In certain embodiments, R¹ is phenyl substituted with C₁₋₄ fluoroalkyl.

In certain embodiments, R¹ is phenyl substituted with alkyl. In certain embodiments, R¹ is phenyl substituted with C₁₋₄ alkyl. In certain embodiments, R¹ is phenyl substituted with halogen. In certain embodiments, R¹ is phenyl substituted with fluoro.

In certain embodiments, R¹ is hydrogen, methyl, butyl, pentyl, —CH₂CH₂CH(CH₃)₂,

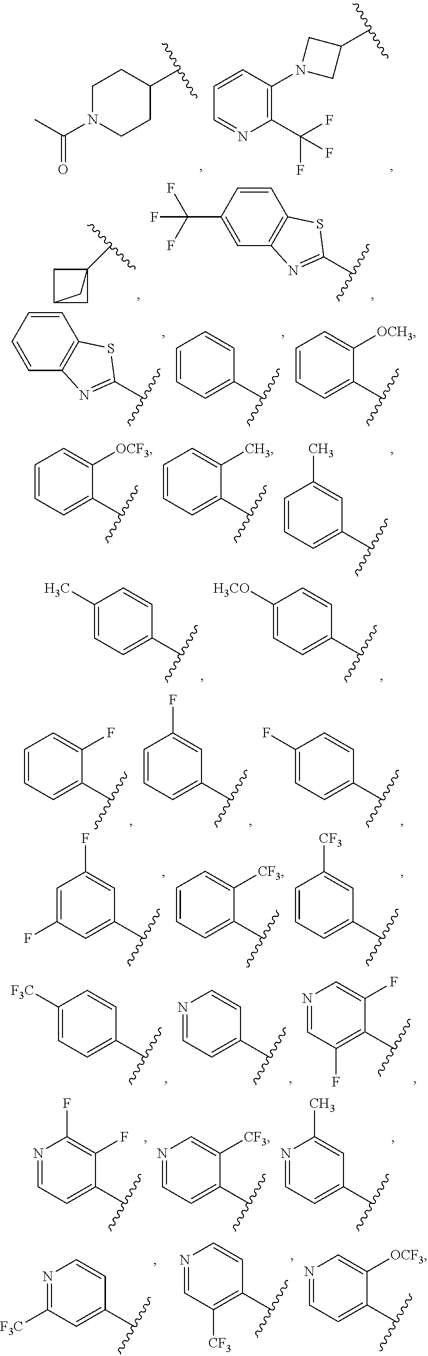

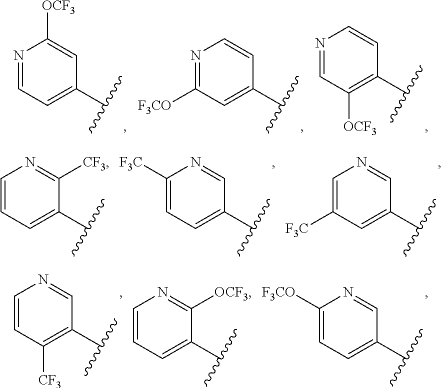

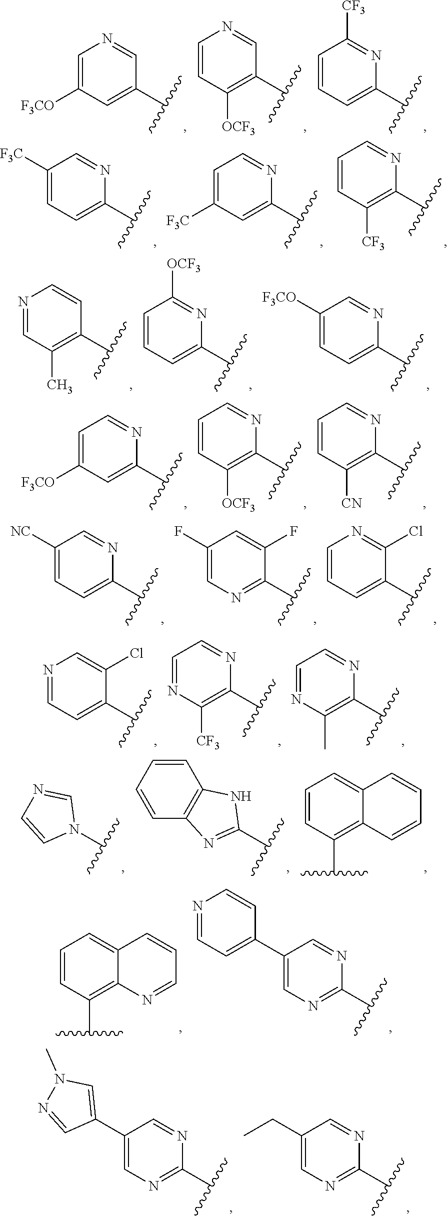

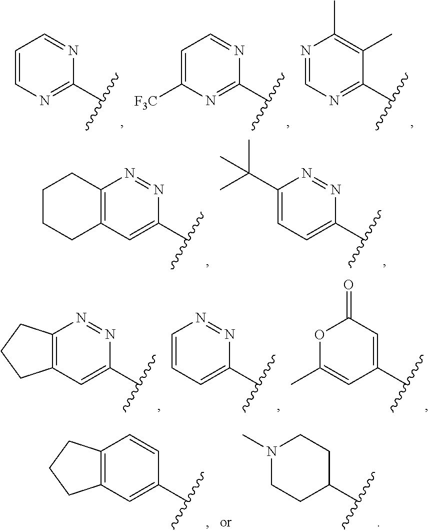

In certain embodiments, R¹ is butyl, pentyl, —CH₂CH₂CH(CH₃)₂,

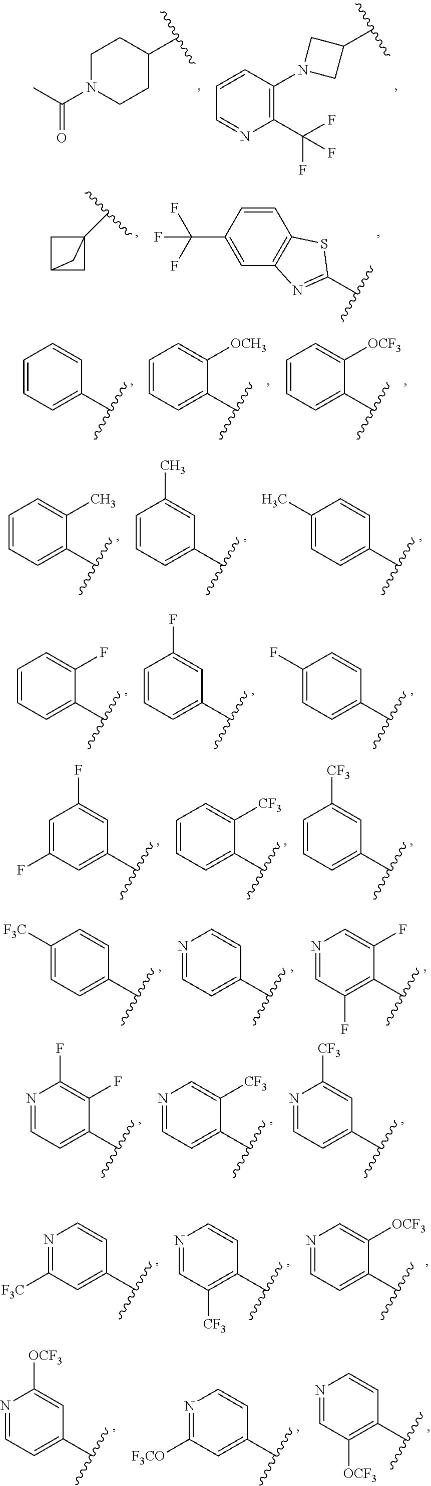

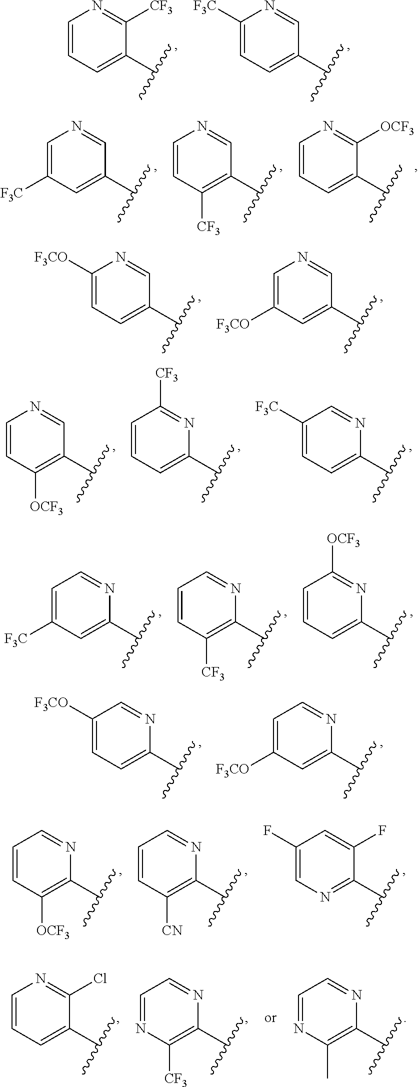

In certain embodiments, R¹ is

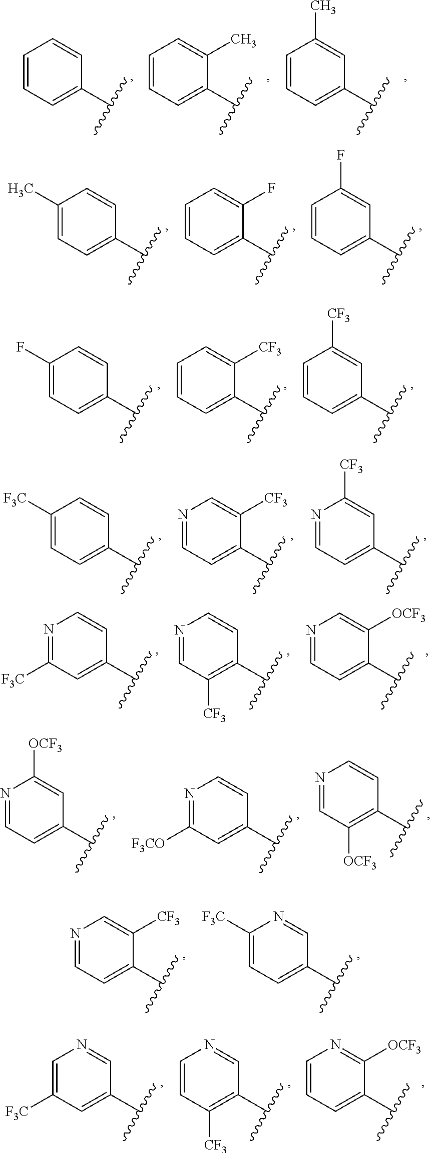

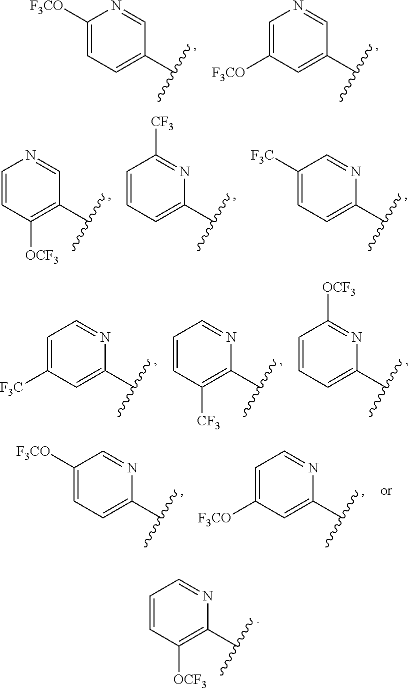

In certain embodiments, R¹ is

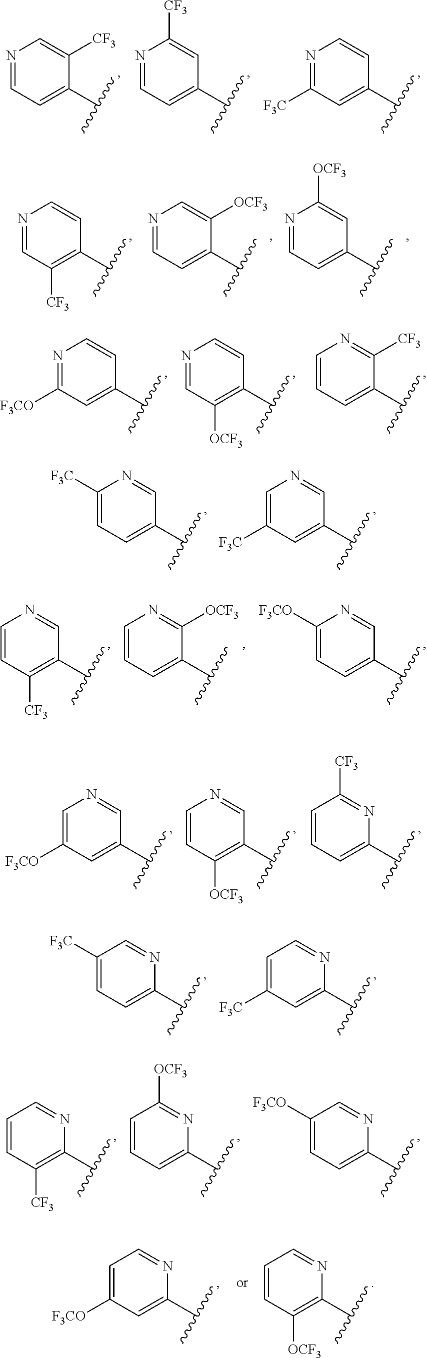

In certain embodiments, R¹ is

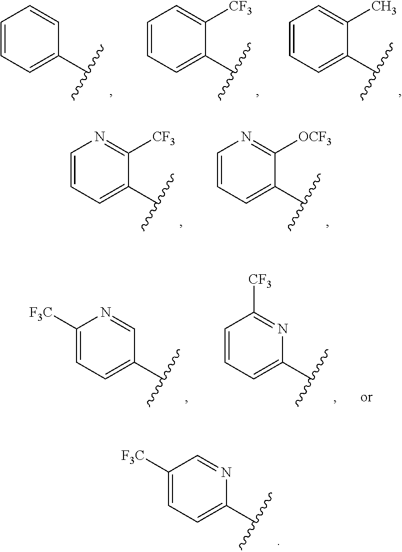

In certain embodiments, R¹ is

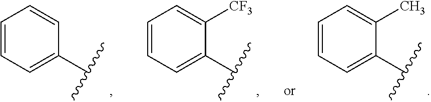

In certain embodiments, R¹ is

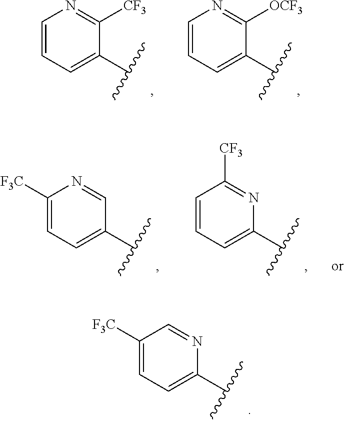 [SMILES: *c1cccnc1C(F)(F)F ; *c1cccnc1OC(F)(F)F ; *c1ccc(C(F)(F)F)nc1 ; *c1cccc(C(F)(F)F)n1 ; *c1ccc(C(F)(F)F)cn1]

As described herein, G is a bond, —S(O)₂—, —NR²—, —CH₂CH₂O—, —CH₂O—, —O— or —CR²R³—. In certain embodiments, G is —S(O)₂—, —NR²—, —CH₂CH₂O—, —CH₂O—, —O— or —CR²R³—. In certain embodiments, G is —O— or —CR²R³—.

In certain embodiments, G is —NR²—. In certain embodiments, G is —CH₂CH₂O—. In certain embodiments, G is —CH₂O—. In certain embodiments, G is —O—. In certain embodiments, G is —CR²R³—. In certain embodiments, G is —CH₂— or —CH(CH₃)—. In certain embodiments, G is —CH₂—. In certain embodiments, G is —CH(CH₃)—.

R² and R³

As described herein, R² and R³ are each independently hydrogen, halogen, or substituted or unsubstituted alkyl, or R² and R³ on the same carbon form with that carbon a carbonyl. In certain embodiments, R² and R³ are each independently hydrogen, halogen, or substituted or unsubstituted alkyl.

In certain embodiments, R² and R³ are each independently hydrogen, or substituted or unsubstituted alkyl. In certain embodiments, R² and R³ are each independently hydrogen, or substituted or unsubstituted C₁₋₄ alkyl. In certain embodiments, R² and R³ are each independently hydrogen, or unsubstituted C₁₋₄ alkyl. In certain embodiments, R² and R³ are each independently hydrogen or methyl. In certain embodiments, R² and R³ are each hydrogen. In certain embodiments, R² and R³ on the same carbon form with that carbon a carbonyl.

In certain embodiments, R² is hydrogen, halogen, or substituted or unsubstituted alkyl; and R³ is hydrogen. In certain embodiments, R² is hydrogen, or substituted or unsubstituted alkyl; and R³ is hydrogen. In certain embodiments, R² is substituted or unsubstituted alkyl; and R³ is hydrogen. In certain embodiments, R² is unsubstituted alkyl; and R³ is hydrogen. In certain embodiments, R² is unsubstituted C₁₋₄ alkyl; and R³ is hydrogen. In certain embodiments, R² is methyl; and R³ is hydrogen.

n

As described herein, n is 1 or 0. In certain embodiments, n is 1. In certain embodiments, n is 0. In certain embodiments, when n is 0, then then A is

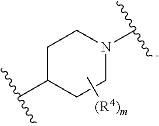

In certain embodiments, when n is 1, then then A is

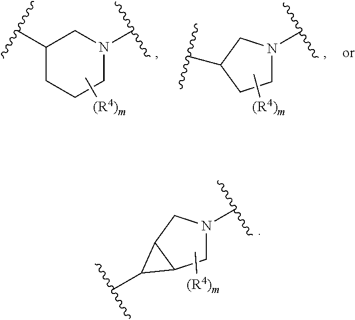

In certain embodiments, when n is 1, then then A is

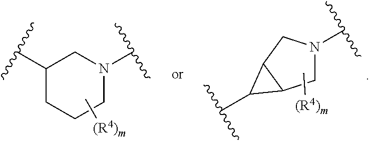

### Ring A

As described herein, A is

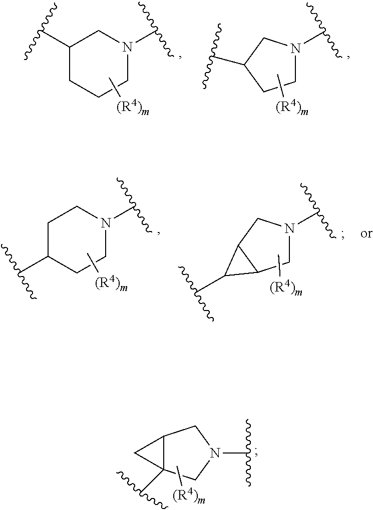

each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl; and m is 0, 1, 2, 3, or 4.

In certain embodiments, A is

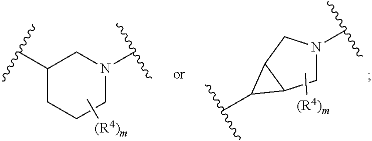

each R⁴ is independently halogen, substituted or unsubstituted alkyl, or two instances of R⁴ on the same carbon form with that carbon a carbonyl; and m is 0, 1, 2, 3, or 4.

In certain embodiments, A is

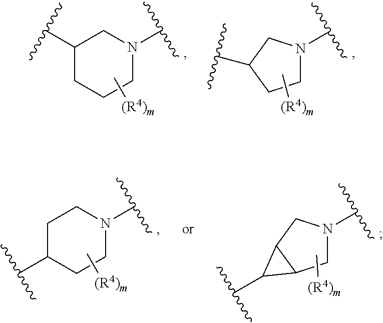

each R⁴ is independently halogen, substituted or unsubstituted alkyl, or two instances of R⁴ on the same carbon form with that carbon a carbonyl; and m is 0, 1, 2, 3, or 4.

In certain embodiments, each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl. In certain embodiments, each R⁴ is independently halogen, substituted or unsubstituted alkyl, or two instances of R⁴ on the same carbon form with that carbon a carbonyl.

In certain embodiments, R⁴ is halogen, or two instances of R⁴ on the same carbon form with that carbon a carbonyl. In certain embodiments, R⁴ is fluoro, or two instances of R⁴ on the same carbon form with that carbon a carbonyl. In certain embodiments, R⁴ is halogen. In certain embodiments, R⁴ is fluoro. In certain embodiments, two instances of R⁴ on the same carbon form with that carbon a carbonyl. In certain embodiments, each R⁴ is independently fluoro, methyl, CH₃OCH₂—, methoxy, difluoromethoxy, or two instances of R⁴ on the same carbon form with that carbon a carbonyl. In certain embodiments, each R⁴ is independently fluoro, methyl, CH₃OCH₂—, methoxy, or difluoromethoxy. In certain embodiments, each R⁴ is independently methyl. In certain embodiments, each R⁴ is independently CH₃OCH₂—. In certain embodiments, each R⁴ is independently methoxy. In certain embodiments, each R⁴ is independently difluoromethoxy.

In certain embodiments, m is 0, 1, 2, or 3. In certain embodiments, m is 0, 1, or 2. In certain embodiments, m is 0 or 2. In certain embodiments, m is 0 or 1. In certain embodiments, m is 1 or 2. In certain embodiments, m is 0. In certain embodiments, m is 2. In certain embodiments, m is 1.

In certain embodiments, R⁴ is halogen, or two instances of R⁴ on the same carbon form with that carbon a carbonyl; and m is 2. In certain embodiments, R⁴ is fluoro, or two instances of R⁴ on the same carbon form with that carbon a carbonyl; and m is 2. In certain embodiments, R⁴ is halogen; and m is 2. In certain embodiments, R⁴ is fluoro; and m is 2. In certain embodiments, two instances of R⁴ on the same carbon form with that carbon a carbonyl; and m is 2.

In certain embodiments, A is

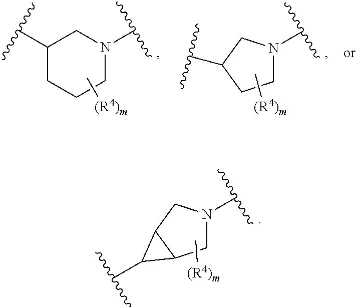

In certain embodiments, A is

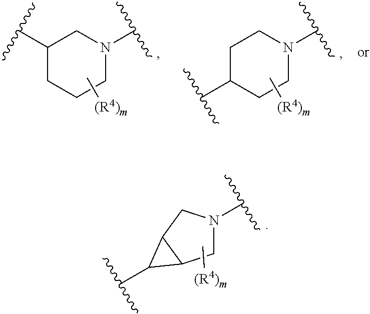

In certain embodiments, A is

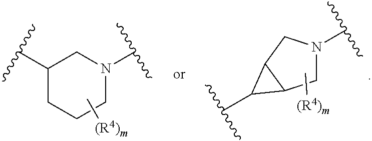

In certain embodiments, A is

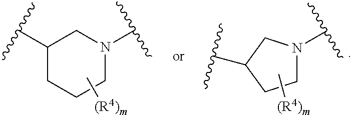

In certain embodiments, A is

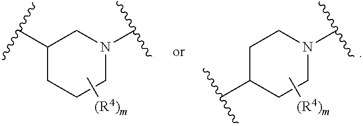

In certain embodiments, A is

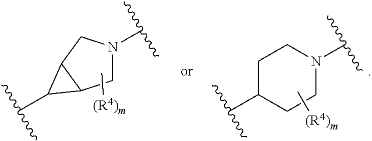

In certain embodiments, A is

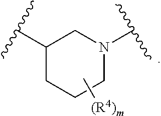

In certain embodiments, A is

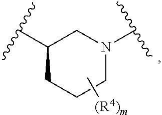

In certain embodiments, A is

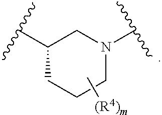

In certain embodiments, A is

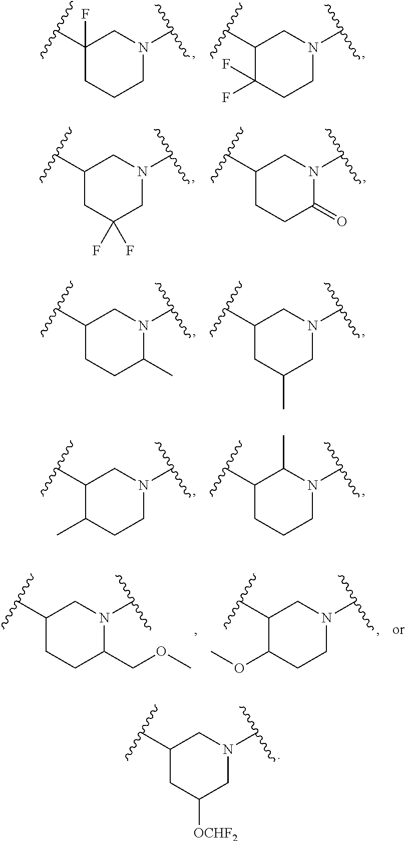

In certain embodiments, A is

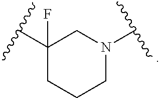 [SMILES: *N1CCCC(*)(F)C1]

In certain embodiments, A is

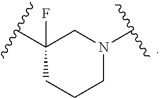 [SMILES: *N1CCC[C@](*)(F)C1]

In certain embodiments, A is

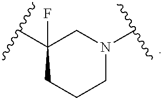 [SMILES: *N1CCC[C@@](*)(F)C1]

In certain embodiments, A is

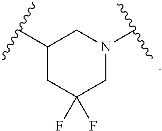 [SMILES: *C1CN(*)CC(F)(F)C1]

In certain embodiments, A is

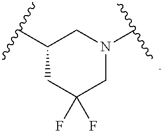 [SMILES: *[C@@H]1CN(*)CC(F)(F)C1]

In certain embodiments, A is

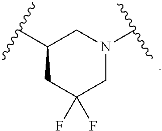 [SMILES: *[C@H]1CN(*)CC(F)(F)C1]

In certain embodiments, A is

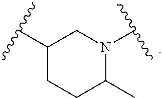 [SMILES: *C1CCC(C)N(*)C1]

In certain embodiments, A is

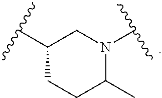

In certain embodiments, A is

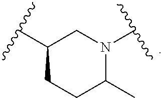

In certain embodiments, A is

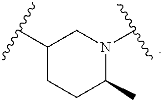 [SMILES: *C1CC[C@H](C)N(*)C1]

In certain embodiments, A is

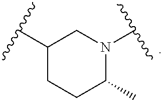 [SMILES: *C1CC[C@@H](C)N(*)C1]

In certain embodiments, A is

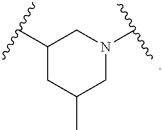 [SMILES: *C1CC(C)CN(*)C1]

In certain embodiments, A is

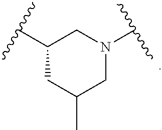

In certain embodiments, A is

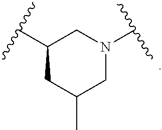

In certain embodiments, A is

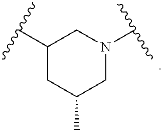 [SMILES: *C1C[C@@H](C)CN(*)C1]

embodiments, A is

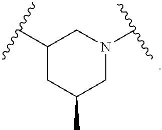 [SMILES: *C1C[C@H](C)CN(*)C1]

In certain embodiments, A is

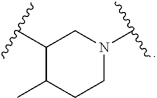 [SMILES: *C1CN(*)CCC1C]

In certain embodiments, A is

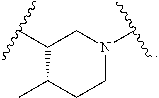

In certain embodiments, A is

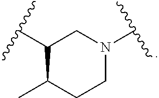

In certain embodiments, A is

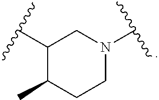 [SMILES: *C1CN(*)CC[C@H]1C]

In certain embodiments, A is

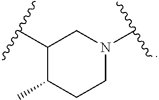 [SMILES: *C1CN(*)CC[C@@H]1C]

In certain embodiments, A is

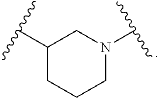 [SMILES: *C1CCCN(*)C1]

In certain embodiments, A is

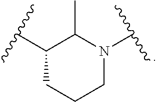

In certain embodiments, A is

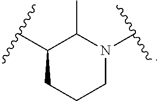

In certain embodiments, A is

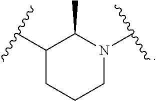 [SMILES: *C1CCCN(*)[C@@H]1C]

In certain embodiments, A is

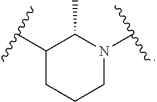 [SMILES: *C1CCCN(*)[C@H]1C]

In certain embodiments, A is

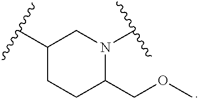 [SMILES: *C1CCC(COC)N(*)C1]

In certain embodiments, A is

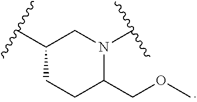

In certain embodiments, A is

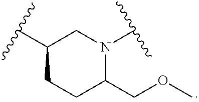

In certain embodiments, A is

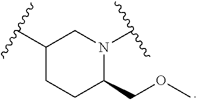 [SMILES: *C1CC[C@H](COC)N(*)C1]

In certain embodiments, A is

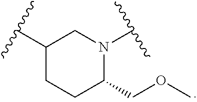 [SMILES: *C1CC[C@@H](COC)N(*)C1]

In certain embodiments, A is

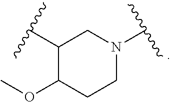 [SMILES: *C1CN(*)CCC1OC]

In certain embodiments, A is

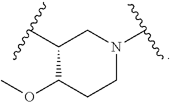

In certain embodiments, A is

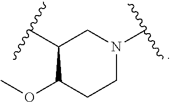

In certain embodiments, A is

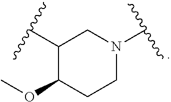 [SMILES: *C1CN(*)CC[C@H]1OC]

In certain embodiments, A is

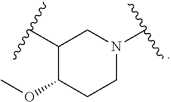 [SMILES: *C1CN(*)CC[C@@H]1OC]

In certain embodiments, A is

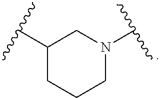 [SMILES: *C1CCCN(*)C1]

In certain embodiments, A is

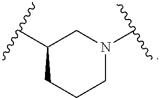 [SMILES: *[C@@H]1CCCN(*)C1]

In certain embodiments, A is

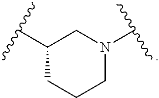 [SMILES: *[C@H]1CCCN(*)C1]

In certain embodiments, A is

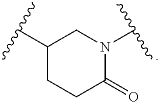 [SMILES: *C1CCC(=O)N(*)C1]

In certain embodiments, A is

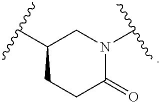 [SMILES: *[C@@H]1CCC(=O)N(*)C1]

In certain embodiments, A is

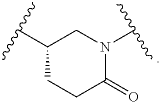 [SMILES: *[C@H]1CCC(=O)N(*)C1]

In certain embodiments, A is

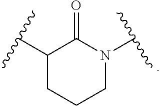 [SMILES: *C1CCCN(*)C1=O]

In certain embodiments, A is

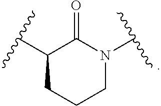 [SMILES: *[C@@H]1CCCN(*)C1=O]

In certain embodiments, A is

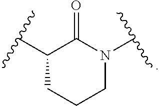 [SMILES: *[C@H]1CCCN(*)C1=O]

In certain embodiments, A is

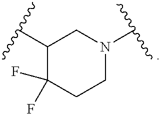 [SMILES: *C1CN(*)CCC1(F)F]

In certain embodiments, A is

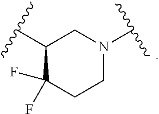 [SMILES: *[C@H]1CN(*)CCC1(F)F]

In certain embodiments, A is

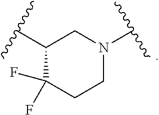 [SMILES: *[C@@H]1CN(*)CCC1(F)F]

In certain embodiments, A is

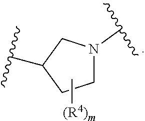

In certain embodiments, A is

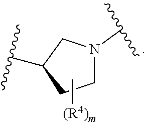

In certain embodiments, A is

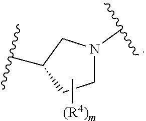

In certain embodiments, A is

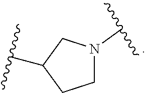 [SMILES: *C1CCN(*)C1]

In certain embodiments, A is

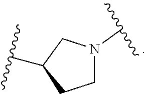 [SMILES: *[C@@H]1CCN(*)C1]

In certain embodiments, A is

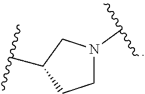 [SMILES: *[C@H]1CCN(*)C1]

In certain embodiments, A is

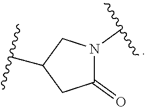 [SMILES: *C1CC(=O)N(*)C1]

In certain embodiments, A is

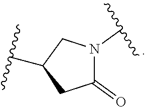 [SMILES: *[C@@H]1CC(=O)N(*)C1]

In certain embodiments, A is

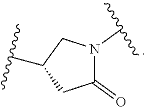 [SMILES: *[C@H]1CC(=O)N(*)C1]

In certain embodiments, A is

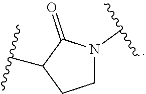 [SMILES: *C1CCN(*)C1=O]

In certain embodiments, A is

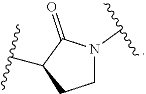 [SMILES: *[C@@H]1CCN(*)C1=O]

In certain embodiments, A is

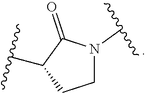 [SMILES: *[C@H]1CCN(*)C1=O]

In certain embodiments, A is

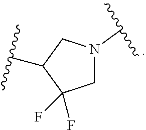 [SMILES: *C1CN(*)CC1(F)F]

In certain embodiments, A is

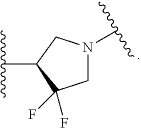 [SMILES: *[C@H]1CN(*)CC1(F)F]

In certain embodiments, A is

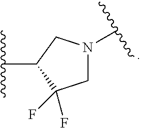 [SMILES: *[C@@H]1CN(*)CC1(F)F]

In certain embodiments, A is

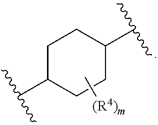

In certain embodiments, A is

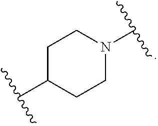 [SMILES: *C1CCN(*)CC1]

In certain embodiments, A is

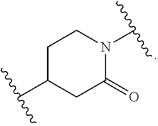 [SMILES: *C1CCN(*)C(=O)C1]

In certain embodiments, A is

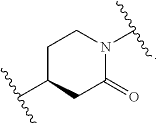 [SMILES: *[C@H]1CCN(*)C(=O)C1]

In certain embodiments, A is

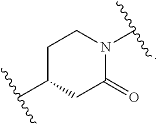 [SMILES: *[C@@H]1CCN(*)C(=O)C1]

In certain embodiments, A is

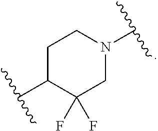 [SMILES: *C1CCN(*)CC1(F)F]

In certain embodiments, A is

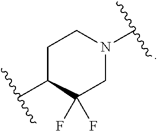 [SMILES: *[C@H]1CCN(*)CC1(F)F]

In certain embodiments, A is

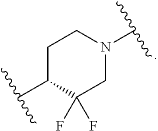 [SMILES: *[C@@H]1CCN(*)CC1(F)F]

In certain embodiments, A is

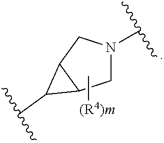

In certain embodiments, A is

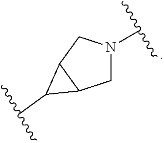 [SMILES: *C1C2CN(*)CC12]

In certain embodiments, A is

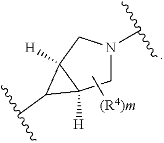

In certain embodiments, A is

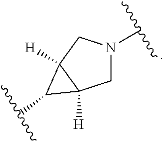 [SMILES: *[C@@H]1[C@H]2CN(*)C[C@@H]12]

In certain embodiments, A is

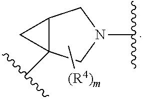

In certain embodiments, A is

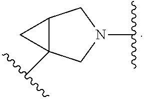 [SMILES: *N1CC2CC2(*)C1]

### L

As described herein, L is a bond, —C(═O)—, —C(═O)CH₂—, —C(═O)CF₂—, —C(═O)CH(Ph)—, —C(═O)CH(iPr)—, —C(═O)CH(Et)—, —C(═O)CH(Me)—, —C(═O)C(CH₃)₂—, —C(═O)CH(OMe)—, —C(═O)CH₂CH₂—, —C(═O)CH₂CH₂CH₂—, —C(═O)CH₂CH₂CH₂O—, —C(═O)CH(CH₃)CH₂—, —C(═O)CH₂O—, —C(═O)CH₂OCH₂—, —C(═O)CH(CH₃)O—, —C(═O)CH₂CH═CH—, —C(═O)NHCH₂CH₂CH₂—, —C(═O)NHCH₂CH₂—, —CH₂—, —CH₂CH₂CH₂—, —CH₂C(CH₃)₂—, —C(═O)NH—, or —CH₂C(═O)NH—.

In certain embodiments, L is a bond, —C(═O)—, —C(═O)CH₂—, or —C(═O)CH₂O—.

In certain embodiments, L is a bond or —C(═O)—. In certain embodiments, L is a bond. In certain embodiments, L is-C(═O)—. In certain embodiments, L is —C(═O)CH₂—. In certain embodiments, L is —C(═O)CH₂O—.

### R⁵

As described herein, R⁵ is substituted or unsubstituted heteroaryl, substituted or unsubstituted heterocyclyl, substituted or unsubstituted heteroarylalkyl, substituted or unsubstituted carbocyclyl, substituted or unsubstituted aryl, methyl, ethyl, butyl, pentyl, t-butyl, —CH₂CH₂CH(CH₃)₂, —SCF₃, or —OCH₂CH(CH₃)₂.

In certain embodiments, R⁵ is substituted or unsubstituted heteroaryl, substituted or unsubstituted heterocyclyl, substituted or unsubstituted heteroarylalkyl, substituted or unsubstituted aryl, substituted or unsubstituted carbocyclyl, or substituted or unsubstituted aryloxyalkyl.

In certain embodiments, R⁵ is substituted or unsubstituted pyrazolopyrazinyl, substituted or unsubstituted indolyl, substituted or unsubstituted oxadiazolyl, substituted or unsubstituted triazolyl, or substituted or unsubstituted pyrazinyl.

In certain embodiments, R⁵ is substituted or unsubstituted heteroaryl, substituted or unsubstituted heterocyclyl, substituted or unsubstituted heteroarylalkyl, substituted or unsubstituted carbocyclyl, or substituted or unsubstituted aryloxyalkyl.

In certain embodiments, R⁵ is substituted or unsubstituted heteroaryl. In certain embodiments, R⁵ is substituted or unsubstituted heterocyclyl. In certain embodiments, R⁵ is substituted or unsubstituted heteroarylalkyl. In certain embodiments, R⁵ is substituted or unsubstituted carbocyclyl. In certain embodiments, R⁵ is substituted or unsubstituted aryloxyalkyl.

In certain embodiments, R⁵ is substituted or unsubstituted heteroaryl, substituted or unsubstituted tetrahydropyranyl, substituted or unsubstituted heteroarylalkyl, substituted or unsubstituted cycloalkyl, or substituted or unsubstituted aryloxyalkyl.

In certain embodiments, R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted imidazopyridinyl, substituted or unsubstituted triazolopyridinyl, substituted or unsubstituted pyrazolopyrimidinyl, substituted or unsubstituted pyrrolopyrimidinyl, substituted or unsubstituted chromenonyl, substituted or unsubstituted isochromanyl, substituted or unsubstituted indolyl, substituted or unsubstituted quinoxalinyl, substituted or unsubstituted benzofuranyl, substituted or unsubstituted benzothiophenyl, substituted or unsubstituted benzimidazolyl, substituted or unsubstituted benzoxazolyl, substituted or unsubstituted 5,6,7,8-tetrahydro-[1,2,4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1,4]dioxinyl, substituted or unsubstituted tetrahydronaphthalenyl, substituted or unsubstituted isoquinolinonyl, substituted or unsubstituted quinolinyl, substituted or unsubstituted naphthyridinyl, substituted or unsubstituted naphthyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted imidazolyl, substituted or unsubstituted pyrrolyl, substituted or unsubstituted thiophenyl, substituted or unsubstituted furanyl, substituted or unsubstituted isothiazolyl, substituted or unsubstituted isoxazolyl, substituted or unsubstituted isoxazolonyl, substituted or unsubstituted 3,4-dihydro-1H-pyrrolo[2,1-c][1,4]thiazin-8-yl, substituted or unsubstituted pyrrolidinonyl, substituted or unsubstituted pyrrolidinyl, substituted or unsubstituted 1,4-diazepanyl, substituted or unsubstituted dioxolanonyl, substituted or unsubstituted pyridinyl, substituted or unsubstituted pyrazinyl, substituted or unsubstituted pyridazinyl, substituted or unsubstituted pyrimidinyl, substituted or unsubstituted phenyl, substituted or unsubstituted tetrahydropyranyl, substituted or unsubstituted tetrahydrofuranyl, substituted or unsubstituted morpholinyl, substituted or unsubstituted cyclooctyl, substituted or unsubstituted cyclohexyl, substituted or unsubstituted cyclopentyl, substituted or unsubstituted cyclobutyl, substituted or unsubstituted cyclopropyl, substituted or unsubstituted bicyclo[3.3.1]nonanyl, substituted or unsubstituted bicyclo[2.2.1]heptanyl, substituted or unsubstituted 7-oxaspiro[3.5]non-1-en-2-yl, substituted or unsubstituted hexahydro-1H-cyclopenta[c]furan-5-yl, substituted or unsubstituted adamantyl, substituted or unsubstituted spiro[2.5]octan-4-yl, methyl, ethyl, butyl, pentyl, t-butyl, —CH₂CH₂CH(CH₃)₂, —SCF₃, or —OCH₂CH(CH₃)₂.

In certain embodiments, R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted pyrazolopyrimidinyl, substituted or unsubstituted indolyl, substituted or unsubstituted quinoxalinyl, substituted or unsubstituted benzofuranyl, substituted or unsubstituted 5,6,7,8-tetrahydro-[1,2,4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1,4]dioxinyl, substituted or unsubstituted tetrahydronaphthalenyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted pyrazinyl, substituted or unsubstituted pyrimidinyl, substituted or unsubstituted phenyl, substituted or unsubstituted tetrahydropyranyl, substituted or unsubstituted morpholinyl, or substituted or unsubstituted cyclopentyl.

In certain embodiments, R⁵ is substituted or unsubstituted pyrazolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted heteroarylalkyl, substituted or unsubstituted cycloalkyl, or substituted or unsubstituted aryloxyalkyl.

In certain embodiments, R⁵ is substituted or unsubstituted pyrazolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted heterocyclyl, substituted or unsubstituted heteroarylalkyl, substituted or unsubstituted cycloalkyl, or substituted or unsubstituted aryloxyalkyl.

In certain embodiments, R⁵ is substituted or unsubstituted pyrazolopyrazinyl, substituted or unsubstituted pyrrolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted pyrazolylmethyl, substituted or unsubstituted indolylmethyl, substituted or unsubstituted cyclohexyl, or substituted or unsubstituted phenyloxyalkyl.

In certain embodiments, R⁵ is substituted or unsubstituted pyrazolopyrazinyl, substituted or unsubstituted pyrrolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, or substituted or unsubstituted pyrazinyl. In certain embodiments. In certain embodiments, R⁵ is substituted or unsubstituted pyrazolylmethyl, or substituted or unsubstituted indolylmethyl.

In certain embodiments, R⁵ is substituted or unsubstituted pyrazolopyrazinyl. In certain embodiments, R⁵ is substituted or unsubstituted pyrrolopyrazinyl. In certain embodiments, R⁵ is substituted or unsubstituted chromenonyl. In certain embodiments, R⁵ is substituted or unsubstituted indolyl. In certain embodiments, R⁵ is substituted or unsubstituted oxadiazolyl. In certain embodiments, R⁵ is substituted or unsubstituted pyrazolyl. In certain embodiments, R⁵ is substituted or unsubstituted triazolyl. In certain embodiments, R⁵ is substituted or unsubstituted pyrazinyl. In certain embodiments, R⁵ is substituted or unsubstituted tetrahydropyranyl. In certain embodiments, R⁵ is substituted or unsubstituted pyrazolylmethyl. In certain embodiments, R⁵ is substituted or unsubstituted indolylmethyl. In certain embodiments, R⁵ is substituted or unsubstituted cyclohexyl. In certain embodiments, R⁵ is or substituted or unsubstituted phenyloxyalkyl.

In certain embodiments, R⁵ is substituted pyrazolopyrazinyl, substituted pyrrolopyrazinyl, substituted chromenonyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl.

In certain embodiments, R⁵ is substituted pyrazolopyrazinyl, substituted pyrrolopyrazinyl, substituted chromenonyl, substituted indolyl, substituted oxadiazolyl, substituted pyrazolyl, substituted triazolyl, or substituted pyrazinyl. In certain embodiments, R⁵ is substituted tetrahydropyranyl. In certain embodiments, R⁵ is substituted pyrazolylmethyl or unsubstituted indolylmethyl. In certain embodiments, R⁵ is substituted cyclohexyl. In certain embodiments, R⁵ is substituted phenyloxypropyl.

In certain embodiments, R⁵ is substituted pyrazolopyrazinyl, substituted pyrrolopyrazinyl, substituted chromenonyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl, wherein each substituted R⁵ is substituted with haloalkyl, cycloalkyl, heteroaryl, aryl, halogen, arylalkyl, alkoxy, alkyl, heterocyclylalkyl, or heterocyclyl.

In certain embodiments, R⁵ is pyrazolopyrazinyl substituted with alkyl or haloalkyl. In certain embodiments, R⁵ is pyrrolopyrazinyl substituted with alkyl or haloalkyl. In certain embodiments, R⁵ is chromenonyl substituted with halogen. In certain embodiments, R⁵ is indolyl substituted with heterocyclylalkyl or heterocyclyl. In certain embodiments, R⁵ is oxadiazolyl substituted with cycloalkyl. In certain embodiments, R⁵ is pyrazolyl substituted with arylalkyl. In certain embodiments, R⁵ is triazolyl substituted with aryl. In certain embodiments, R⁵ is pyrazinyl substituted with heteroaryl. In certain embodiments, R⁵ is tetrahydropyranyl substituted with aryl. In certain embodiments, R⁵ is pyrazolylmethyl substituted with alkyl or cycloalkyl. In certain embodiments, R⁵ is unsubstituted indolylmethyl. In certain embodiments, R⁵ is cyclohexyl substituted with haloalkyl. In certain embodiments, R⁵ is bicyclo[2.2.1]heptanyl substituted with haloalkyl. In certain embodiments, R⁵ is phenyloxypropyl substituted with alkoxy.

In certain embodiments, R⁵ is

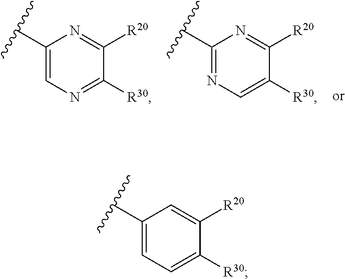

wherein R²⁰ and R³⁰ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R²⁰ and R³⁰ together with the atoms to which they are attached form a substituted or unsubstituted carbocyclyl, substituted or unsubstituted heterocyclyl, substituted or unsubstituted aryl, or substituted or unsubstituted heteroaryl.

In certain embodiments, A is

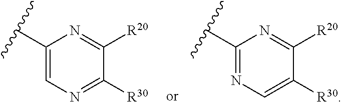

In certain embodiments, A is

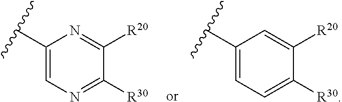

In certain embodiments, A is

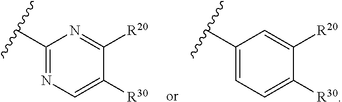

In certain embodiments, A is

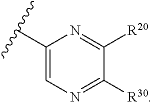

In certain embodiments, A is

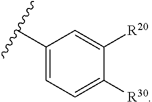

In certain embodiments, A is

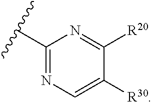

In certain embodiments, R²⁰ and R³⁰ are each independently hydrogen or substituted or unsubstituted heteroaryl; or R²⁰ and R³⁰ together with the atoms to which they are attached form a substituted or unsubstituted aryl, or substituted or unsubstituted heteroaryl.

In certain embodiments, R²⁰ is substituted or unsubstituted heteroaryl. In certain embodiments, R²⁰ is unsubstituted heteroaryl. In certain embodiments, R²⁰ is substituted or unsubstituted thiadizaolyl. In certain embodiments, R²⁰ is unsubstituted thiadizaolyl. In certain embodiments, R³⁰ is hydrogen.

In certain embodiments, R²⁰ is substituted or unsubstituted heteroaryl; and R³⁰ is hydrogen. In certain embodiments, R²⁰ is unsubstituted heteroaryl; and R³⁰ is hydrogen. In certain embodiments, R²⁰ is substituted or unsubstituted thiadizaolyl; and R³⁰ is hydrogen. In certain embodiments, R²⁰ is unsubstituted thiadizaolyl; and R³⁰ is hydrogen.

In certain embodiments, R²⁰ and R³⁰ together with the atoms to which they are attached form a substituted or unsubstituted aryl, or substituted or unsubstituted heteroaryl.

In certain embodiments, R²⁰ and R³⁰ together with the atoms to which they are attached form a substituted or unsubstituted aryl. In certain embodiments, R²⁰ and R³⁰ together with the atoms to which they are attached form a substituted or unsubstituted phenyl. In certain embodiments, R²⁰ and R³⁰ together with the atoms to which they are attached form a substituted phenyl. In certain embodiments, R²⁰ and R³⁰ together with the atoms to which they are attached form an unsubstituted phenyl.

In certain embodiments, R²⁰ and R³⁰ together with the atoms to which they are attached form a substituted or unsubstituted heteroaryl. In certain embodiments, R²⁰ and R³⁰ together with the atoms to which they are attached form a substituted or unsubstituted imidazolyl, substituted or unsubstituted pyrrolyl, or substituted or unsubstituted pyrazolyl. In certain embodiments, R²⁰ and R³⁰ together with the atoms to which they are attached form a substituted or unsubstituted pyrrolyl or substituted or unsubstituted pyrazolyl. In certain embodiments, R²⁰ and R³⁰ together with the atoms to which they are attached form a substituted imidazolyl, substituted pyrrolyl, or substituted pyrazolyl. In certain embodiments, R²⁰ and R³⁰ together with the atoms to which they are attached form a substituted pyrrolyl or substituted pyrazolyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with substituted or unsubstituted alkyl, or substituted or unsubstituted heterocyclyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with substituted or unsubstituted alkyl. In certain embodiments, R²⁰ and R³⁰ together with the atoms to which they are attached form a substituted pyrrolyl or substituted pyrazolyl, wherein the pyrrolyl or pyrazolyl is substituted with substituted or unsubstituted alkyl, or substituted or unsubstituted heterocyclyl. In certain embodiments, R²⁰ and R³⁰ together with the atoms to which they are attached form a substituted pyrrolyl or substituted pyrazolyl, wherein the pyrrolyl or pyrazolyl is substituted with substituted or unsubstituted alkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with unsubstituted alkyl, heterocyclylalkyl, heterocyclyl, or haloalkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with unsubstituted alkyl or haloalkyl. In certain embodiments, R²⁰ and R³⁰ together with the atoms to which they are attached form a substituted pyrrolyl or substituted pyrazolyl, wherein the pyrrolyl or pyrazolyl is substituted with unsubstituted alkyl, heterocyclylalkyl, heterocyclyl, or haloalkyl. In certain embodiments, R²⁰ and R³⁰ together with the atoms to which they are attached form a substituted pyrrolyl or substituted pyrazolyl, wherein the pyrrolyl or pyrazolyl is substituted with unsubstituted alkyl or haloalkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with unsubstituted C₁₋₄ alkyl, 4-5 membered heterocyclyl C₁₋₄ alkyl, 4-5 membered heterocyclyl, or C₁₋₄ haloalkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with unsubstituted C₁₋₄ alkyl or C₁₋₄ haloalkyl. In certain embodiments, R²⁰ and R³⁰ together with the atoms to which they are attached form a substituted pyrrolyl or substituted pyrazolyl, wherein the pyrrolyl or pyrazolyl is substituted with unsubstituted C₁₋₄ alkyl, 4-5 membered heterocyclyl C₁₋₄ alkyl, 4-5 membered heterocyclyl, or C₁₋₄ haloalkyl. In certain embodiments, R²⁰ and R³⁰ together with the atoms to which they are attached form a substituted pyrrolyl or substituted pyrazolyl, wherein the pyrrolyl or pyrazolyl is substituted with unsubstituted C₁₋₄ alkyl or C₁₋₄ haloalkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with unsubstituted C₁₋₄ alkyl. In certain embodiments, R²⁰ and R³⁰ together with the atoms to which they are attached form a substituted pyrrolyl or substituted pyrazolyl, wherein the pyrrolyl or pyrazolyl is substituted with unsubstituted C₁₋₄ alkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with C₁₋₄ haloalkyl. In certain embodiments, R²⁰ and R³⁰ together with the atoms to which they are attached form a substituted pyrrolyl or substituted pyrazolyl, wherein the pyrrolyl or pyrazolyl is substituted with C₁₋₄ haloalkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with 4-5 membered heterocyclyl C₁₋₄ alkyl. In certain embodiments, R²⁰ and R³⁰ together with the atoms to which they are attached form a substituted pyrrolyl or substituted pyrazolyl, wherein the pyrrolyl or pyrazolyl is substituted with 4-5 membered heterocyclyl C₁₋₄ alkyl. In certain embodiments, R²⁰ and R³⁰ together with the atoms to which they are attached form a substituted imidazolyl, substituted pyrrolyl, or substituted pyrazolyl, wherein the imidazolyl, pyrrolyl, or pyrazolyl is substituted with 4-5 membered heterocyclyl. In certain embodiments, R²⁰ and R³⁰ together with the atoms to which they are attached form a substituted pyrrolyl or substituted pyrazolyl, wherein the pyrrolyl or pyrazolyl is substituted with 4-5 membered heterocyclyl.

In certain embodiments, R²⁰ and R³⁰ together with the atoms to which they are attached form a substituted or unsubstituted pyrazolyl. In certain embodiments, R²⁰ and R³⁰ together with the atoms to which they are attached form a substituted pyrazolyl. In certain embodiments, R²⁰ and R³⁰ together with the atoms to which they are attached form a substituted pyrazolyl, wherein the pyrazolyl is substituted with substituted or unsubstituted alkyl, or substituted or unsubstituted heterocyclyl. In certain embodiments, R²⁰ and R³⁰ together with the atoms to which they are attached form a substituted pyrazolyl, wherein the pyrazolyl is substituted with substituted or unsubstituted alkyl. In certain embodiments, R²⁰ and R³⁰ together with the atoms to which they are attached form a substituted pyrazolyl, wherein the pyrazolyl is substituted with unsubstituted alkyl, heterocyclylalkyl, heterocyclyl, or haloalkyl. In certain embodiments, R²⁰ and R³⁰ together with the atoms to which they are attached form a substituted pyrazolyl, wherein the pyrazolyl is substituted with unsubstituted alkyl or haloalkyl. In certain embodiments, R²⁰ and R³⁰ together with the atoms to which they are attached form a substituted pyrazolyl, wherein the pyrazolyl is substituted with unsubstituted C₁₋₄ alkyl, 4-5 membered heterocyclyl C₁₋₄ alkyl, 4-5 membered heterocyclyl, or C₁₋₄ haloalkyl. In certain embodiments, R²⁰ and R³⁰ together with the atoms to which they are attached form a substituted pyrazolyl, wherein the pyrazolyl is substituted with unsubstituted C₁₋₄ alkyl or C₁₋₄ haloalkyl. In certain embodiments, R²⁰ and R³⁰ together with the atoms to which they are attached form a substituted pyrazolyl, wherein the pyrazolyl is substituted with unsubstituted C₁₋₄ alkyl. In certain embodiments, R²⁰ and R³⁰ together with the atoms to which they are attached form a substituted pyrazolyl, wherein the pyrazolyl is substituted with C₁₋₄ haloalkyl. In certain embodiments, R²⁰ and R³⁰ together with the atoms to which they are attached form a substituted pyrazolyl, wherein the pyrazolyl is substituted with 4-5 membered heterocyclyl. In certain embodiments, R²⁰ and R³⁰ together with the atoms to which they are attached form a substituted pyrazolyl, wherein the pyrazolyl is substituted with 4-5 membered heterocyclyl C₁₋₄ alkyl.

In certain embodiments, R²⁰ and R³⁰ together with the atoms to which they are attached form a substituted or unsubstituted pyrrolyl. In certain embodiments, R²⁰ and R³⁰ together with the atoms to which they are attached form a substituted pyrrolyl. In certain embodiments, R²⁰ and R³⁰ together with the atoms to which they are attached form a substituted, wherein the pyrrolyl is substituted with substituted or unsubstituted heterocyclyl, or substituted or unsubstituted alkyl. In certain embodiments, R²⁰ and R³⁰ together with the atoms to which they are attached form a substituted, wherein the pyrrolyl is substituted with substituted or unsubstituted alkyl. In certain embodiments, R²⁰ and R³⁰ together with the atoms to which they are attached form a substituted pyrrolyl, wherein the pyrrolyl is substituted with heterocyclyl, unsubstituted alkyl, or haloalkyl. In certain embodiments, R²⁰ and R³⁰ together with the atoms to which they are attached form a substituted pyrrolyl, wherein the pyrrolyl is substituted with unsubstituted alkyl or haloalkyl. In certain embodiments, R²⁰ and R³⁰ together with the atoms to which they are attached form a substituted pyrrolyl, wherein the pyrrolyl is substituted with 4-5 membered heterocyclyl, unsubstituted C₁₋₄ alkyl, or C₁₋₄ haloalkyl. In certain embodiments, R²⁰ and R³⁰ together with the atoms to which they are attached form a substituted pyrrolyl, wherein the pyrrolyl is substituted with unsubstituted C₁₋₄ alkyl or C₁₋₄ haloalkyl. In certain embodiments, R²⁰ and R³⁰ together with the atoms to which they are attached form a substituted pyrrolyl, wherein the pyrrolyl is substituted with unsubstituted C₁₋₄ alkyl. In certain embodiments, R²⁰ and R³⁰ together with the atoms to which they are attached form a substituted pyrrolyl, wherein the pyrrolyl is substituted with C₁₋₄ haloalkyl. In certain embodiments, R²⁰ and R³⁰ together with the atoms to which they are attached form a substituted pyrrolyl, wherein the pyrrolyl is substituted with 4-5 membered heterocyclyl.

In certain embodiments, R⁵ is

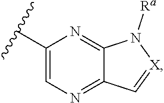

wherein X is N or CH; and R^{a} is substituted or unsubstituted alkyl, or substituted or unsubstituted heterocyclyl. In certain embodiments, R⁵ is

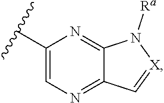

wherein X is N or CH; and R^{a} is substituted or unsubstituted heterocyclyl. In certain embodiments, R⁵ is

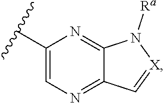

wherein X is N or CH; and R^{a} is substituted or unsubstituted alkyl. In certain embodiments, R⁵ is

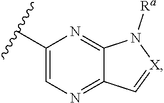

wherein X is N or CH; and R^{a} is haloalkyl or alkyl. In certain embodiments, R⁵ is

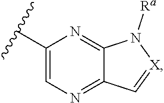

wherein X is N or CH; and R^{a} is C₁₋₄ haloalkyl or C₁₋₄ alkyl. In certain embodiments, R⁵ is

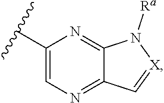

wherein X is N; and R^{a} is substituted or unsubstituted heterocyclyl. In certain embodiments, R⁵ is

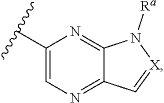

wherein X is N; and R^{a} is substituted or unsubstituted alkyl. In certain embodiments, R⁵ is

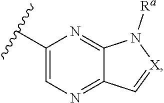

wherein X is N; and R^{a} is haloalkyl or alkyl. In certain embodiments, R⁵ is

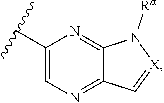

wherein X is N; and R^{a} is C₁₋₄ haloalkyl or C₁₋₄ alkyl. In certain embodiments, R⁵ is

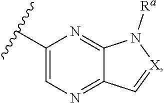

wherein X is CH; and R^{a} is substituted or unsubstituted alkyl. In certain embodiments, R⁵ is

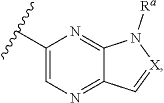

wherein X is CH; and R^{a} is haloalkyl or alkyl. In certain embodiments, R⁵ is

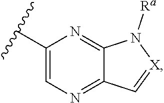

wherein X is CH; and R^{a} is C₁₋₄ haloalkyl or C₁₋₄ alkyl.

In certain embodiments, X is N or CH; and R^{a} is substituted or unsubstituted alkyl. In certain embodiments, X is N or CH; and R^{a} is heterocyclyl, haloalkyl, or alkyl. In certain embodiments, X is N or CH; and R^{a} is haloalkyl or alkyl. In certain embodiments, X is N or CH; and R^{a} is 4-5 membered heterocyclyl, fluoroalkyl, or alkyl. In certain embodiments, X is N or CH; and R^{a} is fluoroalkyl or alkyl. In certain embodiments, X is N or CH; and R^{a} is 4-5 membered heterocyclyl, C₁₋₄ haloalkyl, or C₁₋₄ alkyl. In certain embodiments, X is N or CH; and R^{a} is C₁₋₄ haloalkyl or C₁₋₄ alkyl. In certain embodiments, X is N or CH; and R^{a} is 4-membered heterocyclyl, C₁₋₄ fluoroalkyl, or C₁₋₄ alkyl. In certain embodiments, X is N or CH; and R^{a} is C₁₋₄ fluoroalkyl or C₁₋₄ alkyl. In certain embodiments, X is N; and R^{a} is substituted or unsubstituted alkyl, or substituted or unsubstituted heterocyclyl. In certain embodiments, X is N; and R^{a} is substituted or unsubstituted alkyl. In certain embodiments, X is N; and R^{a} is heterocyclyl, haloalkyl, or alkyl. In certain embodiments, X is N; and R^{a} is haloalkyl or alkyl. In certain embodiments, X is N; and R^{a} is 4-5 membered heterocyclyl, fluoroalkyl, or alkyl. In certain embodiments, X is N; and R^{a} is fluoroalkyl or alkyl. In certain embodiments, X is N; and R^{a} is C₁₋₄ haloalkyl or C₁₋₄ alkyl. In certain embodiments, X is N or CH; and R^{a} is 4-membered heterocyclyl, C₁₋₄ fluoroalkyl, or C₁₋₄ alkyl. In certain embodiments, X is N; and R^{a} is C₁₋₄ fluoroalkyl or C₁₋₄ alkyl. In certain embodiments, X is CH; and R^{a} is substituted or unsubstituted alkyl. In certain embodiments, X is CH; and R^{a} is 4-5 membered heterocyclylalkyl. 4-5 membered heterocyclyl, haloalkyl or alkyl. In certain embodiments, X is CH; and R^{a} is haloalkyl or alkyl. In certain embodiments, X is CH; and R^{a} is 4-5 membered heterocyclyl C₁₋₄ alkyl, 4-5 membered heterocyclyl, C₁₋₄ haloalkyl or C₁₋₄ alkyl. In certain embodiments, X is CH; and R^{a} is C₁₋₄ haloalkyl or C₁₋₄ alkyl. In certain embodiments, X is CH; and R^{a} is 4-5 membered heterocyclylalkyl, 4-5 membered heterocyclyl, fluoroalkyl or alkyl. In certain embodiments, X is CH; and R^{a} is fluoroalkyl or alkyl. In certain embodiments, X is CH; and R^{a} is 4-membered heterocyclyl C₁₋₄ alkyl, 4-membered heterocyclyl, C₁₋₄ fluoroalkyl or C₁₋₄ alkyl. In certain embodiments, X is CH; and R^{a} is C₁₋₄ fluoroalkyl or C₁₋₄ alkyl. In certain embodiments, X is CH; and R^{a} is C₁₋₄ alkyl. In certain embodiments, X is CH; and R^{a} is ethyl. In certain embodiments, X is CH; and R^{a} is oxetanyl. In certain embodiments, X is CH; and R^{a} is oxetanylmethyl.

In certain embodiments, R⁵ is

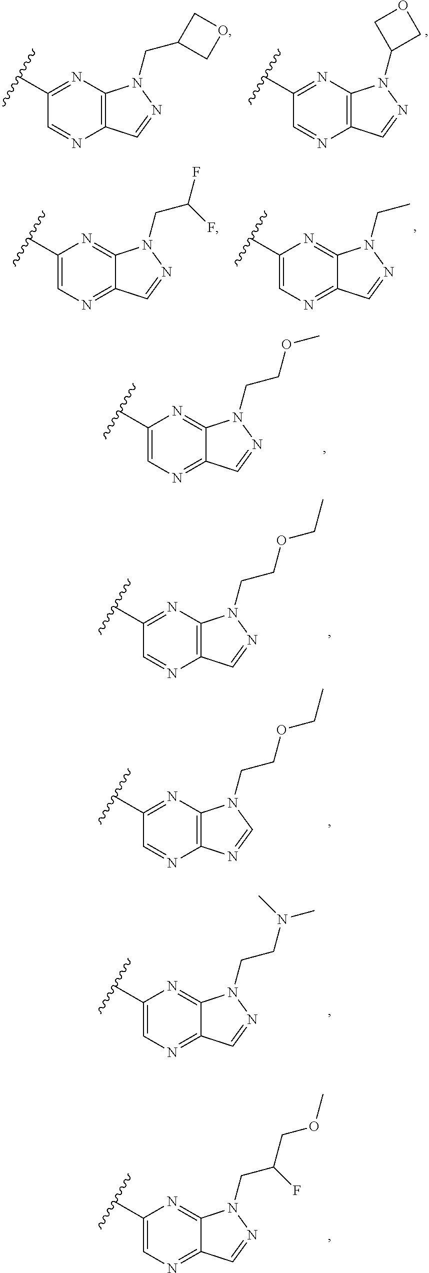

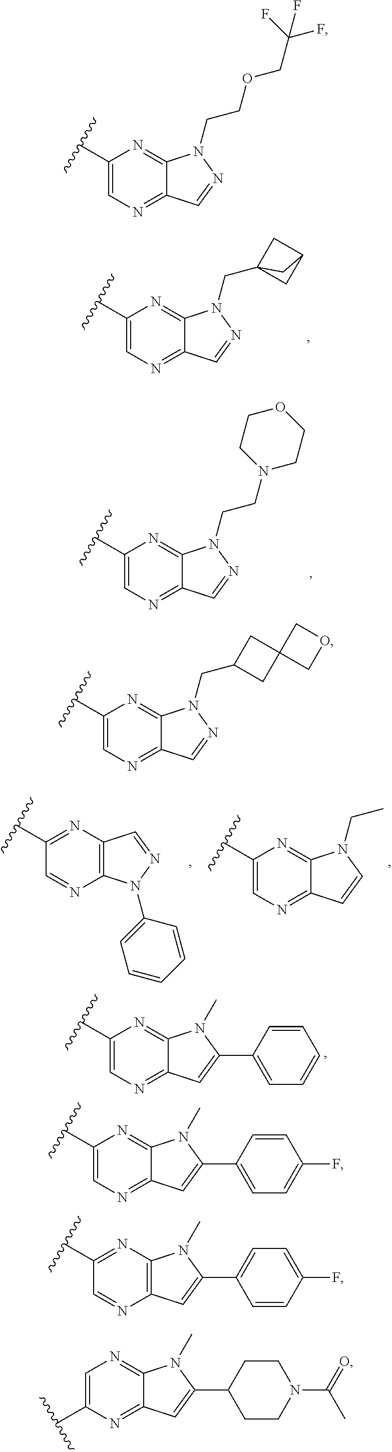

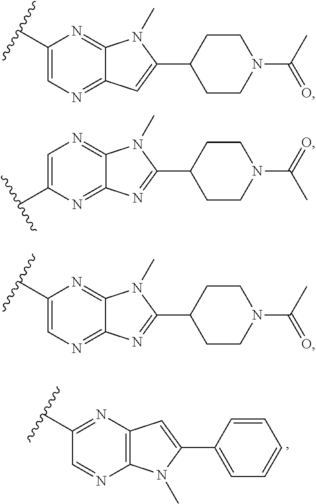

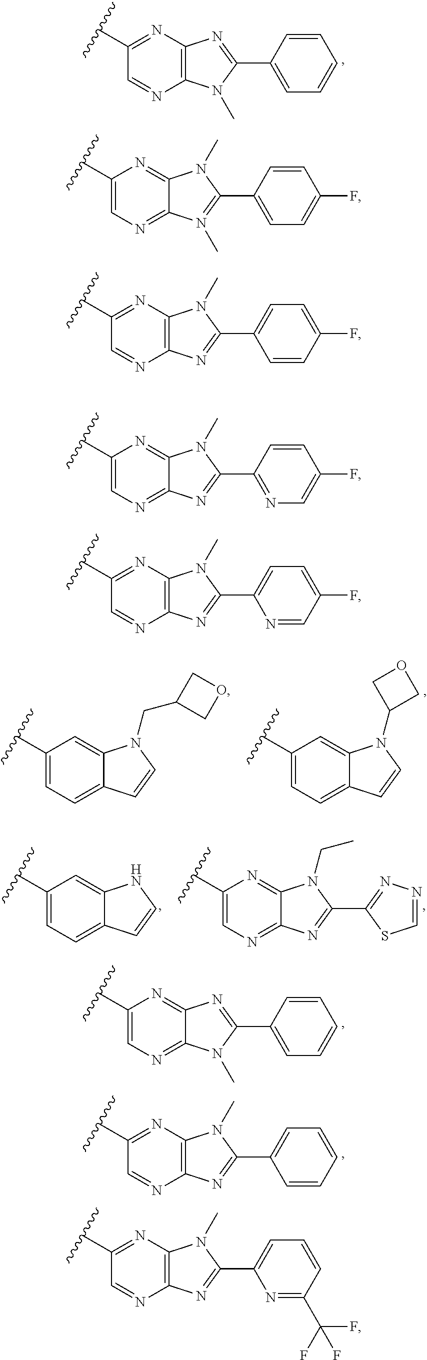

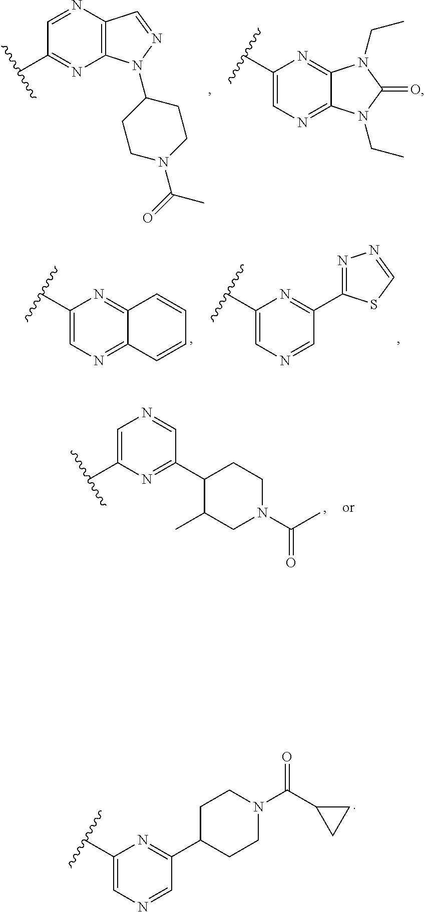

In certain embodiments, R⁵ is

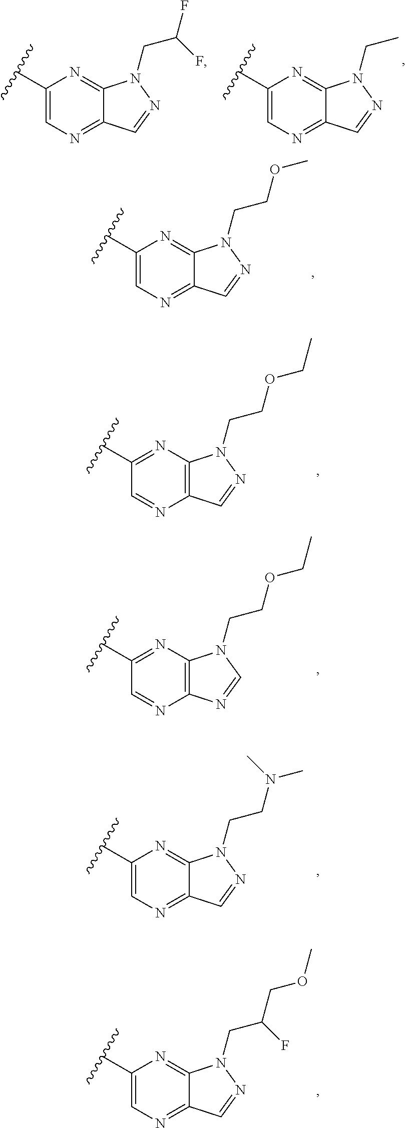

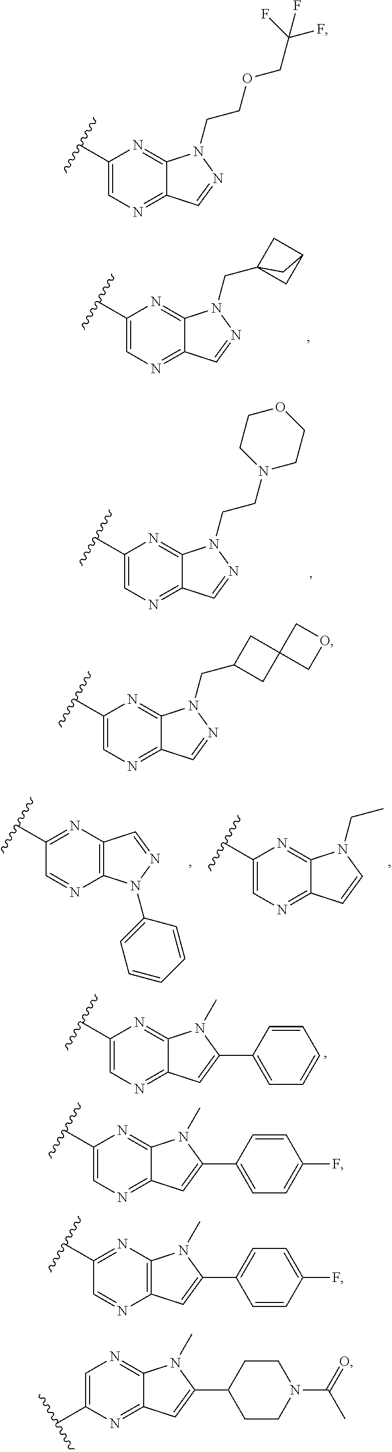

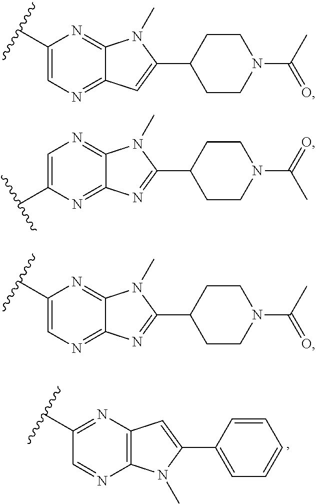

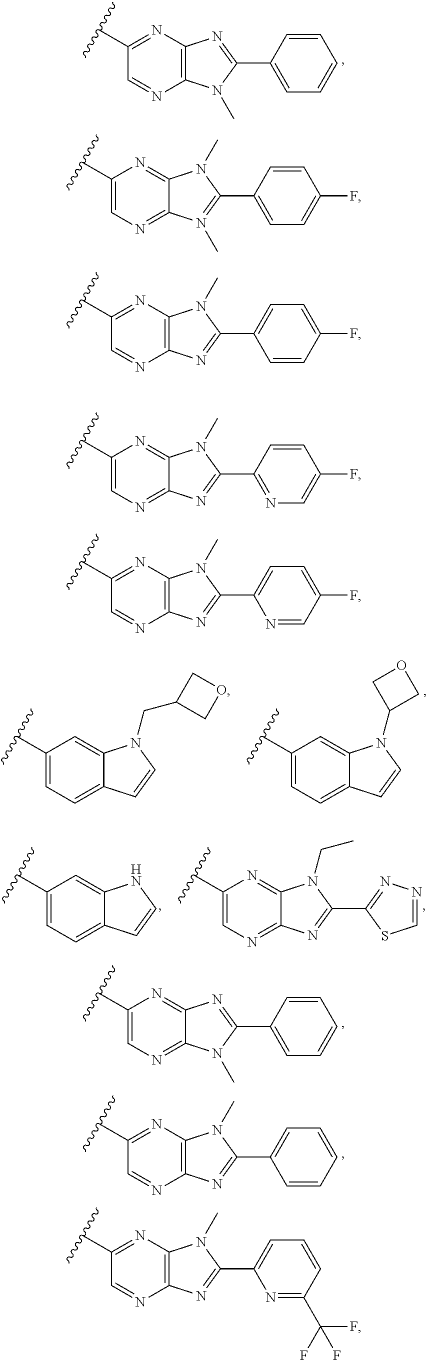

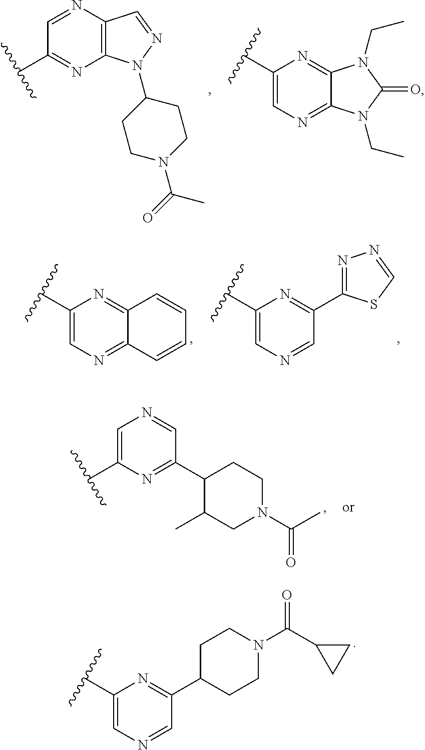

In certain embodiments, R⁵ is

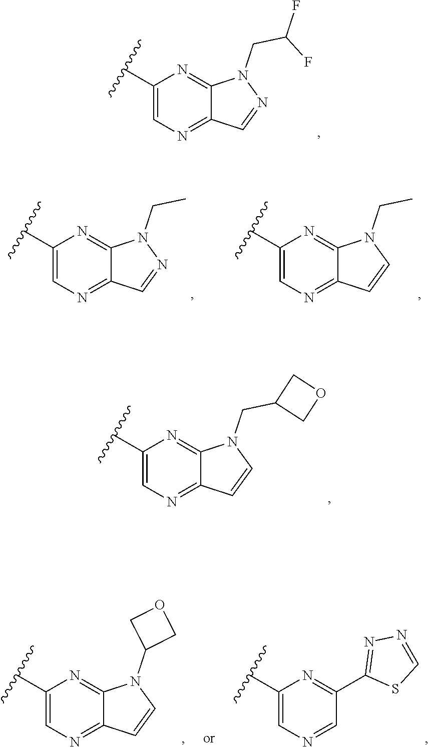 [SMILES: *c1cnc2cnn(CC(F)F)c2n1 ; *c1cnc2cnn(CC)c2n1 ; *c1cnc2ccn(CC)c2n1 ; *c1cnc2ccn(CC3COC3)c2n1 ; *c1cnc2ccn(C3COC3)c2n1 ; *c1cncc(-c2nncs2)n1]

In certain embodiments, R⁵ is

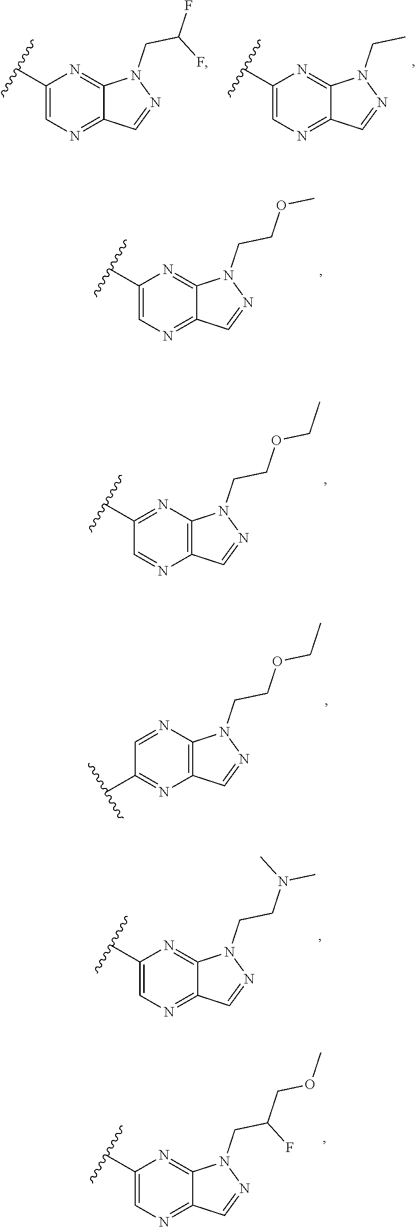

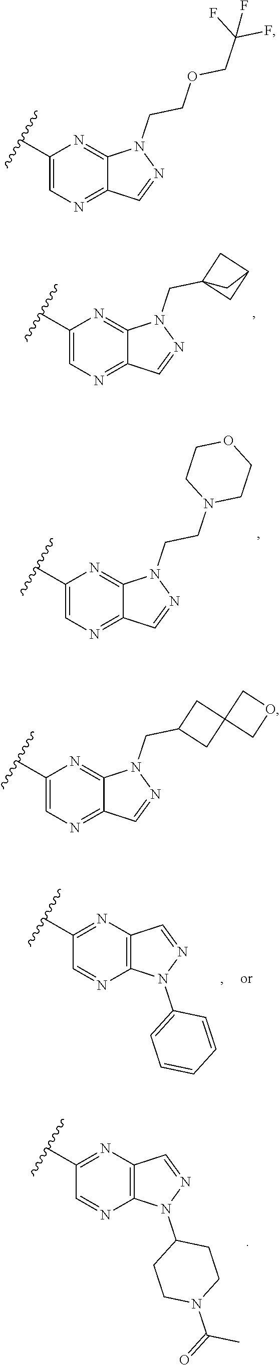 [SMILES: *c1cnc2cnn(CCOCC(F)(F)F)c2n1 ; *c1cnc2cnn(CC34CC(C3)C4)c2n1 ; *c1cnc2cnn(CCN3CCOCC3)c2n1 ; *c1cnc2cnn(CC3CC4(COC4)C3)c2n1 ; *c1cnc2c(cnn2-c2ccccc2)n1 ; *c1cnc2c(cnn2C2CCN(C(C)=O)CC2)n1]

In certain embodiments, R⁵ is

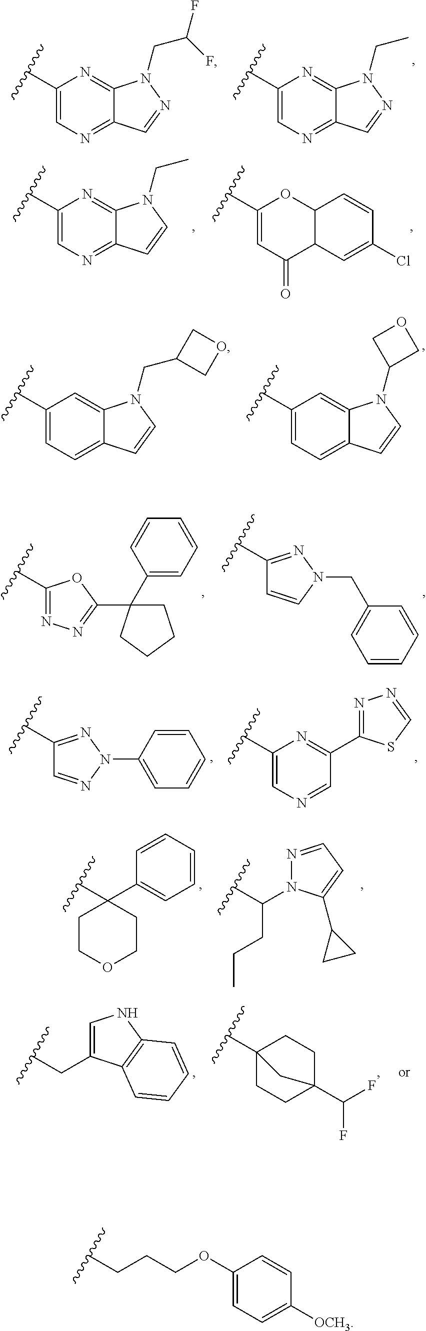

In certain embodiments, R⁵ is

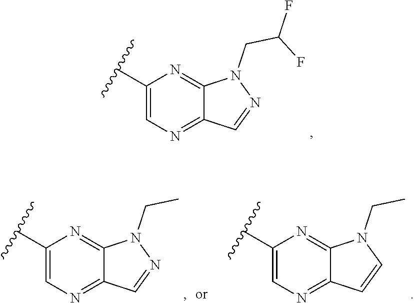 [SMILES: *c1cnc2cnn(CC(F)F)c2n1 ; *c1cnc2cnn(CC)c2n1 ; *c1cnc2ccn(CC)c2n1]

In certain embodiments, R⁵ is

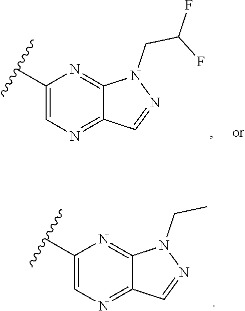 [SMILES: *c1cnc2cnn(CC(F)F)c2n1 ; *c1cnc2cnn(CC)c2n1]

In certain embodiments, R⁵ is

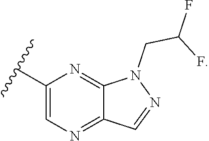 [SMILES: *c1cnc2cnn(CC(F)F)c2n1]

### CERTAIN EMBODIMENTS

In certain embodiments, the compound of Formula (I) is of formula (I):

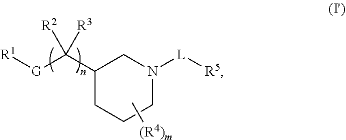

or a pharmaceutically acceptable salt, co-crystal, tautomer, stereoisomer, solvate, hydrate, polymorph, isotopically enriched derivative, or prodrug thereof; wherein R¹, R², R³, R⁴, R⁵, G, L, m and n are as defined herein.

In certain embodiments, the compound of Formula (I) is of Formula (I-a):

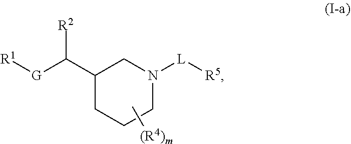

or a pharmaceutically acceptable salt, co-crystal, tautomer, stereoisomer, solvate, hydrate, polymorph, isotopically enriched derivative, or prodrug thereof; wherein R¹, R², R⁴, R⁵, G, L, and m are as defined herein.

In certain embodiments, the compound of Formula (I) is of Formula (I-b):

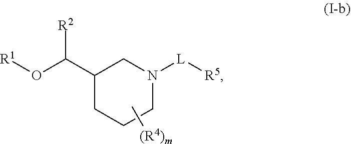

or a pharmaceutically acceptable salt, co-crystal, tautomer, stereoisomer, solvate, hydrate, polymorph, isotopically enriched derivative, or prodrug thereof; wherein R¹, R², R⁴, R⁵, L, and m are as defined herein.

In certain embodiments, the compound of Formula (I) is of Formula (I-c):

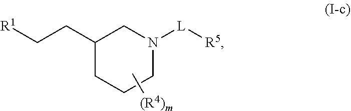

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (I-d):

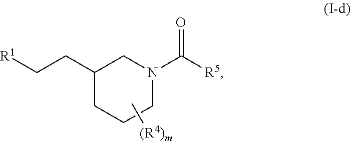

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (I-e):

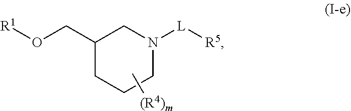

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (I-f):

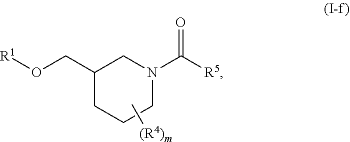

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (I-g):

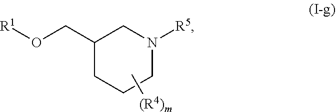

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (I-h):

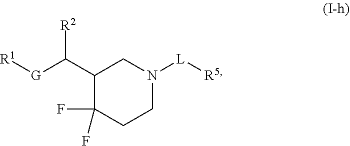

or a pharmaceutically acceptable salt, co-crystal, tautomer, stereoisomer, solvate, hydrate, polymorph, isotopically enriched derivative, or prodrug thereof; wherein R¹, R², R⁵, G, and L are as defined herein.

In certain embodiments, the compound of Formula (I) is of Formula (I-i):

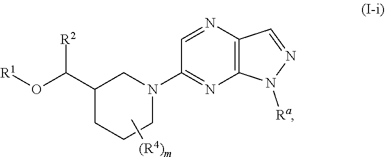

or a pharmaceutically acceptable salt, co-crystal, tautomer, stereoisomer, solvate, hydrate, polymorph, isotopically enriched derivative, or prodrug thereof; wherein R¹, R², R⁴, R^{a}, and m are as defined herein.

In certain embodiments, the compound of Formula (I) is of formula (II):

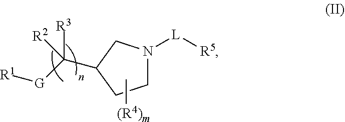

or a pharmaceutically acceptable salt, co-crystal, tautomer, stereoisomer, solvate, hydrate, polymorph, isotopically enriched derivative, or prodrug thereof; wherein R¹, R², R³, R⁴, R⁵, G, L, m and n are as defined herein.

In certain embodiments, the compound of Formula (I) is of Formula (II-a):

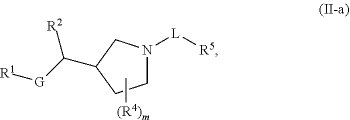

or a pharmaceutically acceptable salt, co-crystal, tautomer, stereoisomer, solvate, hydrate, polymorph, isotopically enriched derivative, or prodrug thereof; wherein R¹, R², R⁴, R⁵, G, L, and m are as defined herein.

In certain embodiments, the compound of Formula (I) is of Formula (II-b):

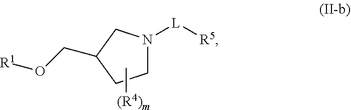

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (II-c):

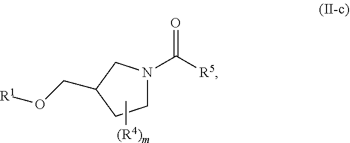

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (II-d):

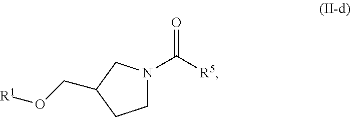

or a pharmaceutically acceptable salt, co-crystal, tautomer, stereoisomer, solvate, hydrate, polymorph, isotopically enriched derivative, or prodrug thereof; wherein R¹ and R⁵ are as defined herein.

In certain embodiments, the compound of Formula (I) is of formula (III):

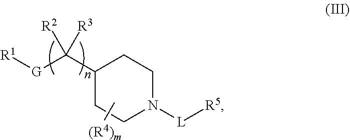

or a pharmaceutically acceptable salt, co-crystal, tautomer, stereoisomer, solvate, hydrate, polymorph, isotopically enriched derivative, or prodrug thereof; wherein R¹, R², R³, R⁴, R⁵, G, L, m and n are as defined herein.

In certain embodiments, the compound of Formula (I) is of Formula (III-a):

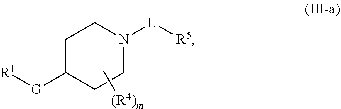

or a pharmaceutically acceptable salt, co-crystal, tautomer, stereoisomer, solvate, hydrate, polymorph, isotopically enriched derivative, or prodrug thereof; wherein R¹, R⁴, R⁵, G, L, and m are as defined herein.

In certain embodiments, the compound of Formula (I) is of Formula (III-b):

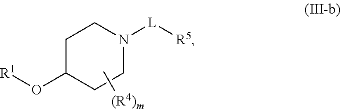

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (III-c):

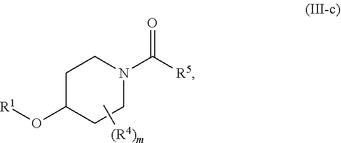

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (III-d):

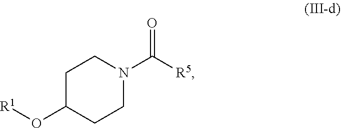

or a pharmaceutically acceptable salt, co-crystal, tautomer, stereoisomer, solvate, hydrate, polymorph, isotopically enriched derivative, or prodrug thereof; wherein R¹ and R⁵ are as defined herein.

In certain embodiments, the compound of Formula (I) is of formula (IV):

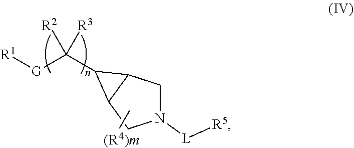

or a pharmaceutically acceptable salt, co-crystal, tautomer, stereoisomer, solvate, hydrate, polymorph, isotopically enriched derivative, or prodrug thereof; wherein R¹, R², R³, R⁴, R⁵, G, L, m, and n are as defined herein.

In certain embodiments, the compound of Formula (I) is of Formula (IV-a):

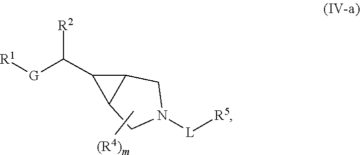

or a pharmaceutically acceptable salt, co-crystal, tautomer, stereoisomer, solvate, hydrate, polymorph, isotopically enriched derivative, or prodrug thereof; wherein R¹, R², R⁴, R⁵, G, L, and m are as defined herein.

In certain embodiments, the compound of Formula (I) is of Formula (IV-b):

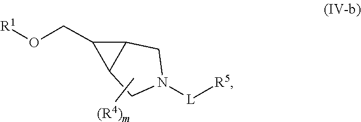

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (IV-c):

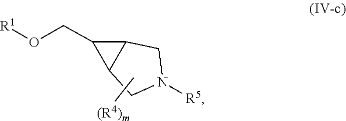

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (IV-d):

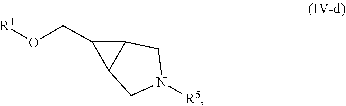

or a pharmaceutically acceptable salt, co-crystal, tautomer, stereoisomer, solvate, hydrate, polymorph, isotopically enriched derivative, or prodrug thereof; wherein R¹ and R⁵ are as defined herein.

In certain embodiments, the compound of Formula (I) is of Formula (IV-e):

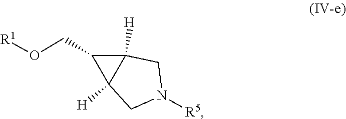

or a pharmaceutically acceptable salt, co-crystal, tautomer, stereoisomer, solvate, hydrate, polymorph, isotopically enriched derivative, or prodrug thereof; wherein R¹ and R⁵ are as defined herein.

In certain embodiments, the compound of Formula (I) is of Formula (V-a):

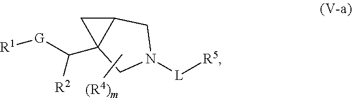

or a pharmaceutically acceptable salt, co-crystal, tautomer, stereoisomer, solvate, hydrate, polymorph, isotopically enriched derivative, or prodrug thereof; wherein R¹, R², R⁴, R⁵, G, L, and m are as defined herein.

In certain embodiments, the compound of Formula (I) is of Formula (V-b):

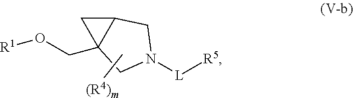

or a pharmaceutically acceptable salt, co-crystal, tautomer, stereoisomer, solvate, hydrate, polymorph, isotopically enriched derivative, or prodrug thereof; wherein R¹, R⁴, R⁵, L, and m are as defined herein.

In certain embodiments, the compound of Formula (I) is of Formula (V-c):

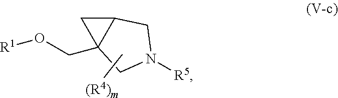

or a pharmaceutically acceptable salt, co-crystal, tautomer, stereoisomer, solvate, hydrate, polymorph, isotopically enriched derivative, or prodrug thereof; wherein R¹, R⁴, R⁵, and m are as defined herein.

In certain embodiments, the compound of Formula (I) is of Formula (V-d):

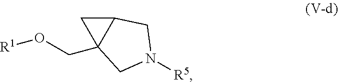

or a pharmaceutically acceptable salt, co-crystal, tautomer, stereoisomer, solvate, hydrate, polymorph, isotopically enriched derivative, or prodrug thereof; wherein R¹ and R⁵ are as defined herein.

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof:

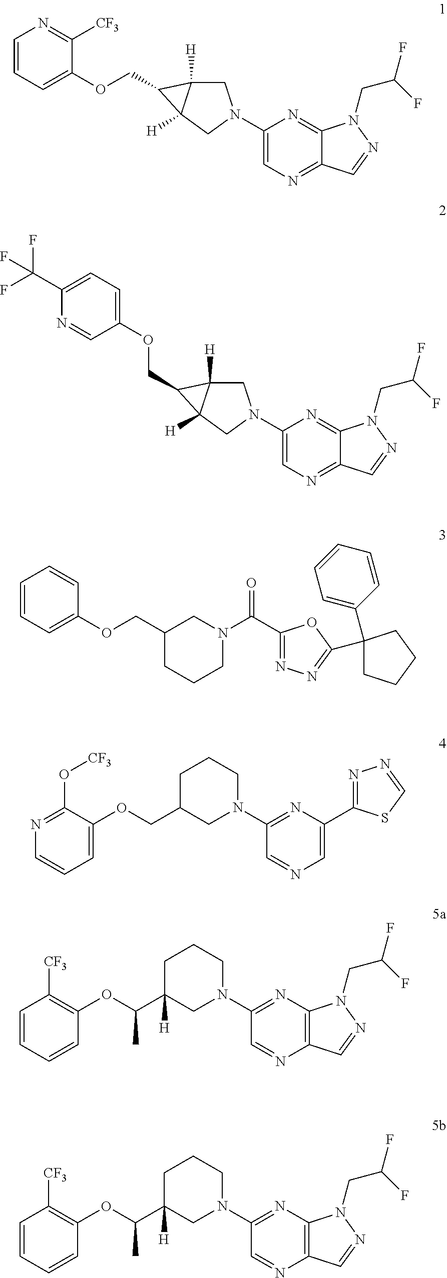 [SMILES: FC(F)Cn1ncc2ncc(N3C[C@@H]4[C@@H](COc5cccnc5C(F)(F)F)[C@@H]4C3)nc21 ; FC(F)Cn1ncc2ncc(N3C[C@@H]4[C@@H](COc5ccc(C(F)(F)F)nc5)[C@@H]4C3)nc21 ; O=C(c1nnc(C2(c3ccccc3)CCCC2)o1)N1CCCC(COc2ccccc2)C1 ; FC(F)(F)Oc1ncccc1OCC1CCCN(c2cncc(-c3nncs3)n2)C1 ; C[C@@H](Oc1ccccc1C(F)(F)F)[C@@H]1CCCN(c2cnc3cnn(CC(F)F)c3n2)C1 ; C[C@@H](Oc1ccccc1C(F)(F)F)[C@@H]1CCCN(c2cnc3cnn(CC(F)F)c3n2)C1]

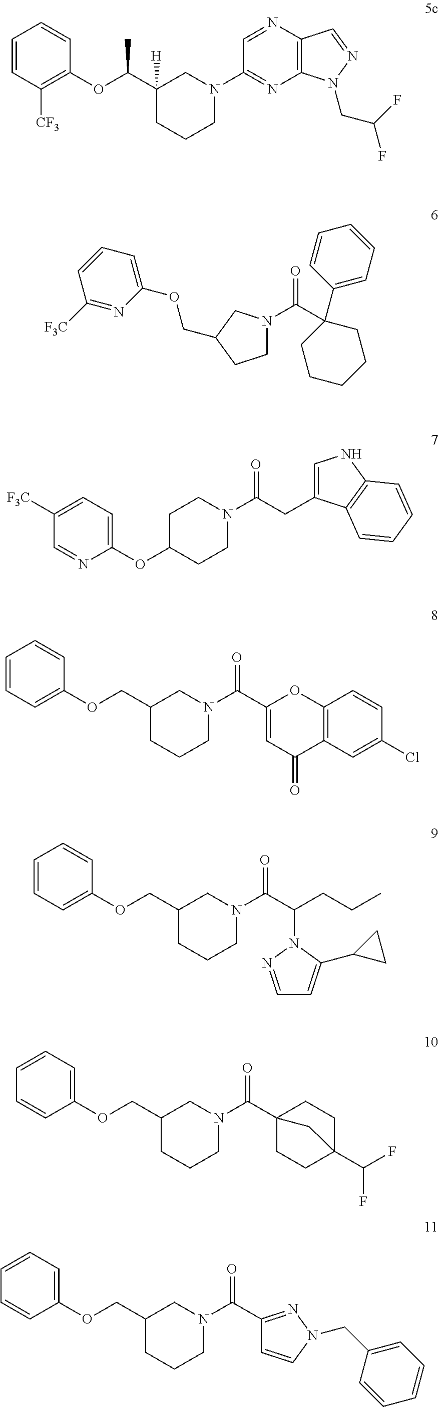

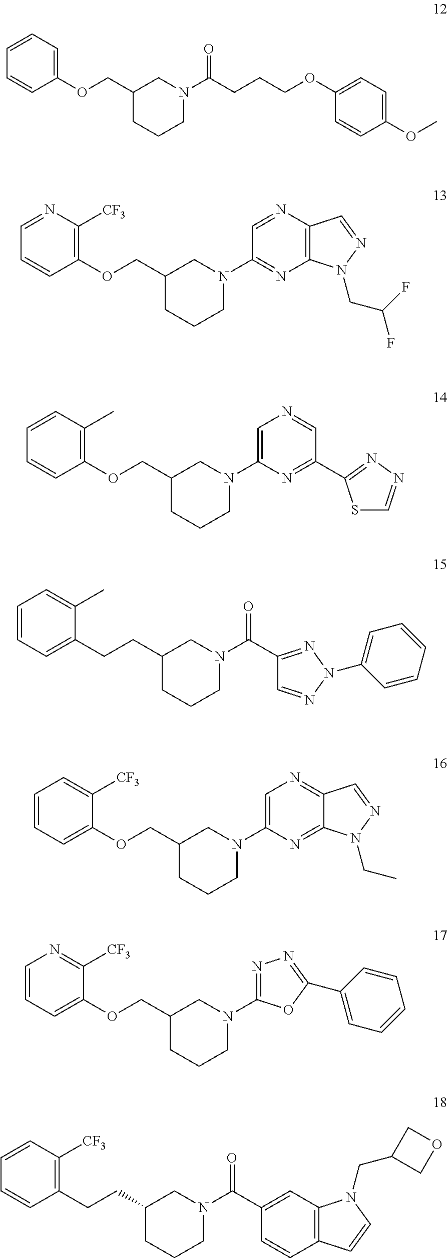

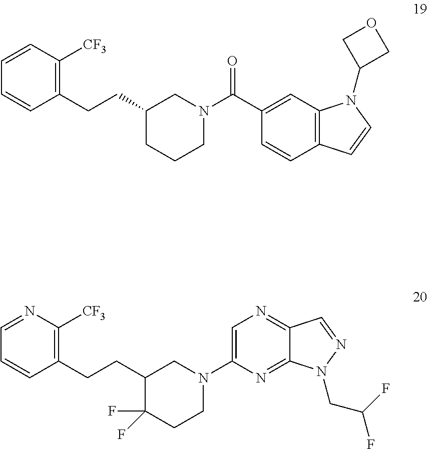 [SMILES: O=C(c1ccc2ccn(C3COC3)c2c1)N1CCC[C@H](CCc2ccccc2C(F)(F)F)C1 ; FC(F)Cn1ncc2ncc(N3CCC(F)(F)C(CCc4cccnc4C(F)(F)F)C3)nc21]

In certain embodiments, the compound of Formula (I) is a compound of Table 1, or a pharmaceutically acceptable salt, co-crystal, tautomer, stereoisomer, solvate, hydrate, polymorph, isotopically enriched derivative, or prodrug thereof.

TABLE 1

| | |
|---|---|
| 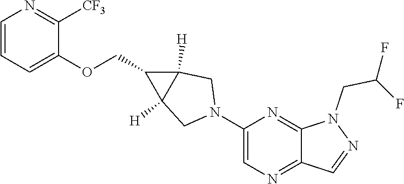 [SMILES: FC(F)Cn1ncc2ncc(N3C[C@@H]4[C@@H](COc5cccnc5C(F)(F)F)[C@@H]4C3)nc21] | 1 |
| 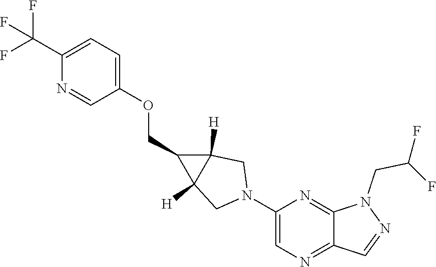 [SMILES: FC(F)Cn1ncc2ncc(N3C[C@@H]4[C@@H](COc5ccc(C(F)(F)F)nc5)[C@@H]4C3)nc21] | 2 |
| 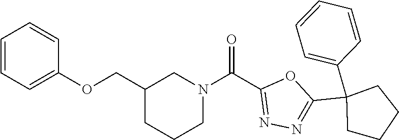 [SMILES: O=C(c1nnc(C2(c3ccccc3)CCCC2)o1)N1CCCC(COc2ccccc2)C1] | 3 |
| 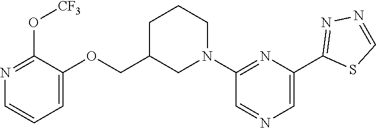 [SMILES: FC(F)(F)Oc1ncccc1OCC1CCCN(c2cncc(-c3nncs3)n2)C1] | 4 |
| 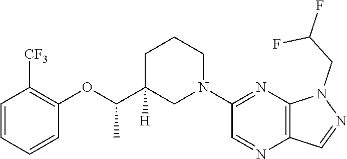 [SMILES: C[C@H](Oc1ccccc1C(F)(F)F)[C@H]1CCCN(c2cnc3cnn(CC(F)F)c3n2)C1] | 5a |
| 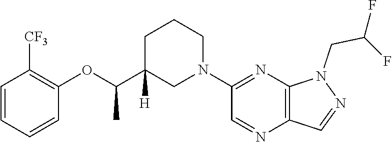 [SMILES: C[C@@H](Oc1ccccc1C(F)(F)F)[C@@H]1CCCN(c2cnc3cnn(CC(F)F)c3n2)C1] | 5b |
| 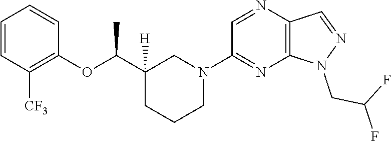 [SMILES: C[C@H](Oc1ccccc1C(F)(F)F)[C@@H]1CCCN(c2cnc3cnn(CC(F)F)c3n2)C1] | 5c |
| 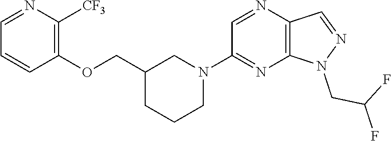 [SMILES: FC(F)Cn1ncc2ncc(N3CCCC(COc4cccnc4C(F)(F)F)C3)nc21] | 13 |
| 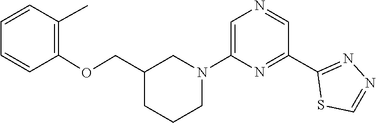 [SMILES: Cc1ccccc1OCC1CCCN(c2cncc(-c3nncs3)n2)C1] | 14 |
| 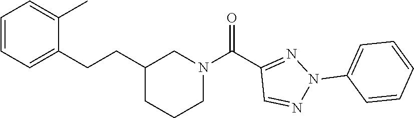 [SMILES: Cc1ccccc1CCC1CCCN(C(=O)c2cnn(-c3ccccc3)n2)C1] | 15 |
| 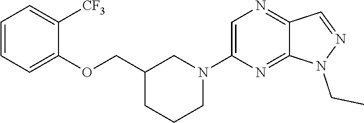 [SMILES: CCn1ncc2ncc(N3CCCC(COc4ccccc4C(F)(F)F)C3)nc21] | 16 |
| 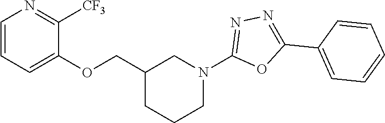 [SMILES: FC(F)(F)c1ncccc1OCC1CCCN(c2nnc(-c3ccccc3)o2)C1] | 17 |
| 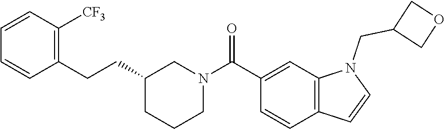 [SMILES: O=C(c1ccc2ccn(CC3COC3)c2c1)N1CCC[C@H](CCc2ccccc2C(F)(F)F)C1] | 18 |
| 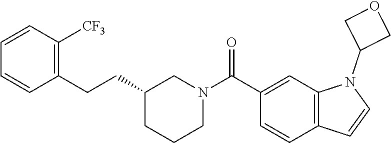 [SMILES: O=C(c1ccc2ccn(C3COC3)c2c1)N1CCC[C@H](CCc2ccccc2C(F)(F)F)C1] | 19 |
| 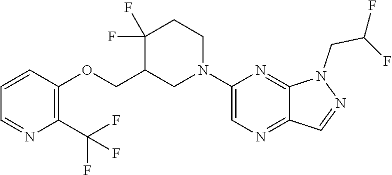 [SMILES: FC(F)Cn1ncc2ncc(N3CCC(F)(F)C(COc4cccnc4C(F)(F)F)C3)nc21] | 20 |
| 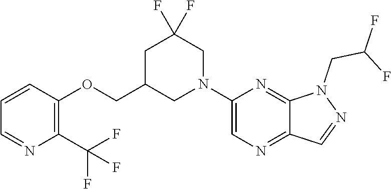 [SMILES: FC(F)Cn1ncc2ncc(N3CC(COc4cccnc4C(F)(F)F)CC(F)(F)C3)nc21] | 21 |
| 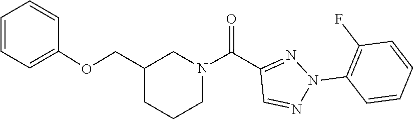 [SMILES: O=C(c1cnn(-c2ccccc2F)n1)N1CCCC(COc2ccccc2)C1] | 22 |
| 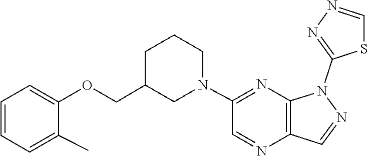 | 23 |
| 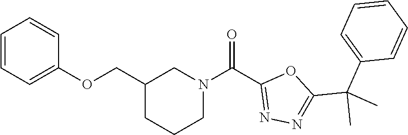 [SMILES: CC(C)(c1ccccc1)c1nnc(C(=O)N2CCCC(COc3ccccc3)C2)o1] | 24 |
| 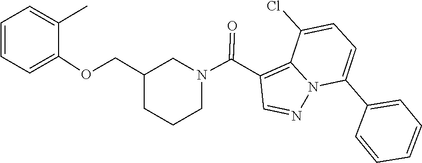 [SMILES: Cc1ccccc1OCC1CCCN(C(=O)c2cnn3c(-c4ccccc4)ccc(Cl)c23)C1] | 25 |
| 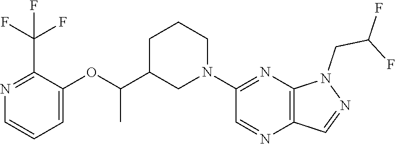 [SMILES: CC(Oc1cccnc1C(F)(F)F)C1CCCN(c2cnc3cnn(CC(F)F)c3n2)C1] | 26 |
| 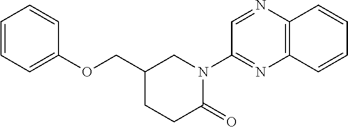 [SMILES: O=C1CCC(COc2ccccc2)CN1c1cnc2ccccc2n1] | 27 |
| 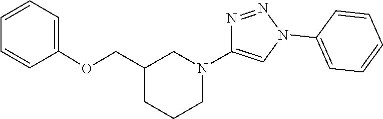 [SMILES: c1ccc(OCC2CCCN(c3cn(-c4ccccc4)nn3)C2)cc1] | 28 |
| 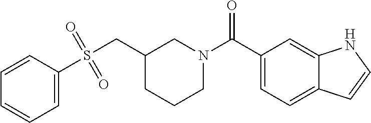 [SMILES: O=C(c1ccc2cc[nH]c2c1)N1CCCC(CS(=O)(=O)c2ccccc2)C1] | 29 |
| 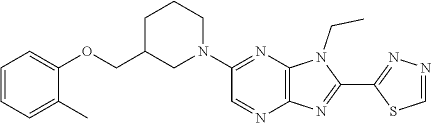 [SMILES: CCn1c(-c2nncs2)nc2ncc(N3CCCC(COc4ccccc4C)C3)nc21] | 30 |
| 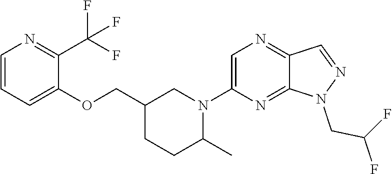 [SMILES: CC1CCC(COc2cccnc2C(F)(F)F)CN1c1cnc2cnn(CC(F)F)c2n1] | 31 |
| 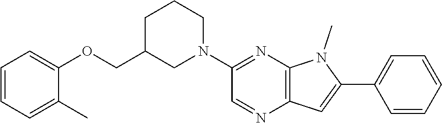 [SMILES: Cc1ccccc1OCC1CCCN(c2cnc3cc(-c4ccccc4)n(C)c3n2)C1] | 32 |
| 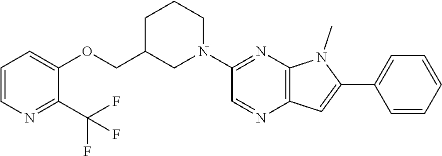 [SMILES: Cn1c(-c2ccccc2)cc2ncc(N3CCCC(COc4cccnc4C(F)(F)F)C3)nc21] | 33 |
| 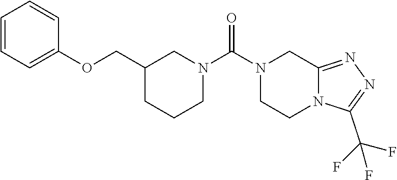 [SMILES: O=C(N1CCn2c(nnc2C(F)(F)F)C1)N1CCCC(COc2ccccc2)C1] | 34 |
| 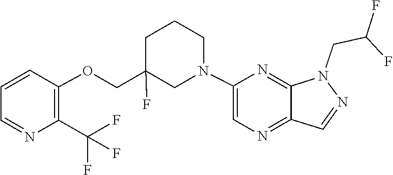 | 35 |
| 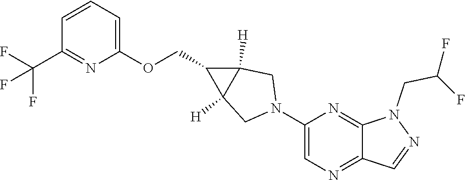 [SMILES: FC(F)Cn1ncc2ncc(N3C[C@@H]4[C@@H](COc5cccc(C(F)(F)F)n5)[C@@H]4C3)nc21] | 36 |
| 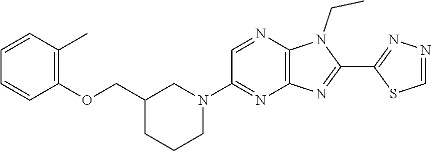 [SMILES: CCn1c(-c2nncs2)nc2nc(N3CCCC(COc4ccccc4C)C3)cnc21] | 37 |
| 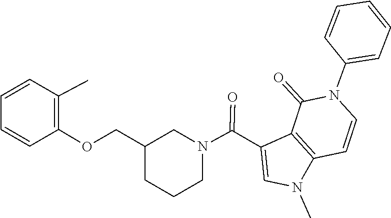 [SMILES: Cc1ccccc1OCC1CCCN(C(=O)c2cn(C)c3ccn(-c4ccccc4)c(=O)c23)C1] | 38 |
| 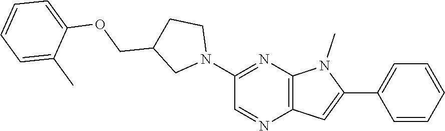 [SMILES: Cc1ccccc1OCC1CCN(c2cnc3cc(-c4ccccc4)n(C)c3n2)C1] | 39 |
| 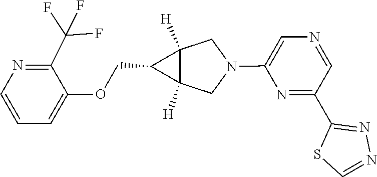 [SMILES: FC(F)(F)c1ncccc1OC[C@@H]1[C@H]2CN(c3cncc(-c4nncs4)n3)C[C@@H]12] | 40 |
| 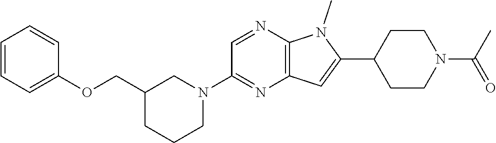 [SMILES: CC(=O)N1CCC(c2cc3nc(N4CCCC(COc5ccccc5)C4)cnc3n2C)CC1] | 41 |
| 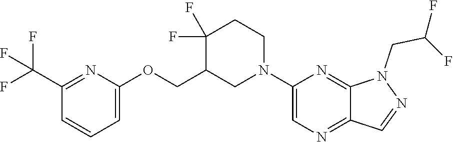 [SMILES: FC(F)Cn1ncc2ncc(N3CCC(F)(F)C(COc4cccc(C(F)(F)F)n4)C3)nc21] | 42 |
| 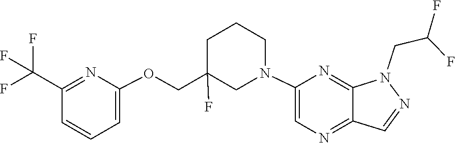 | 43 |
| 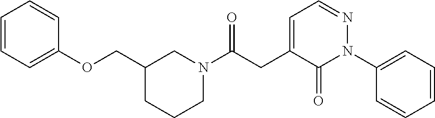 [SMILES: O=C(Cc1ccnn(-c2ccccc2)c1=O)N1CCCC(COc2ccccc2)C1] | 44 |
| 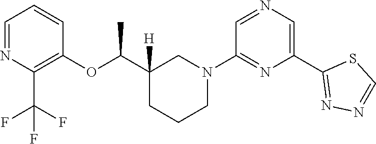 [SMILES: C[C@H](Oc1cccnc1C(F)(F)F)[C@H]1CCCN(c2cncc(-c3nncs3)n2)C1] | 45a |
| 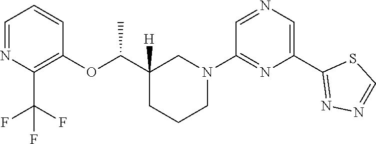 [SMILES: C[C@@H](Oc1cccnc1C(F)(F)F)[C@H]1CCCN(c2cncc(-c3nncs3)n2)C1] | 45b |
| 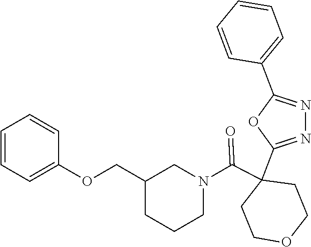 [SMILES: O=C(N1CCCC(COc2ccccc2)C1)C1(c2nnc(-c3ccccc3)o2)CCOCC1] | 46 |
| 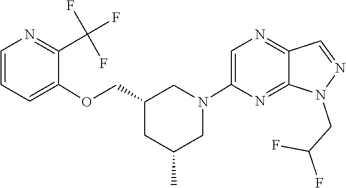 [SMILES: C[C@@H]1C[C@H](COc2cccnc2C(F)(F)F)CN(c2cnc3cnn(CC(F)F)c3n2)C1] | 47a |
| 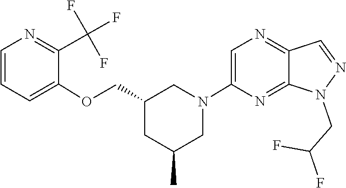 [SMILES: C[C@H]1C[C@H](COc2cccnc2C(F)(F)F)CN(c2cnc3cnn(CC(F)F)c3n2)C1] | 47b |
| 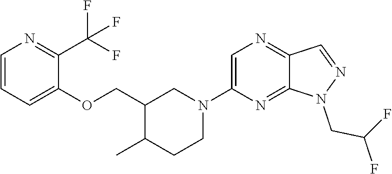 [SMILES: CC1CCN(c2cnc3cnn(CC(F)F)c3n2)CC1COc1cccnc1C(F)(F)F] | 48 |
| 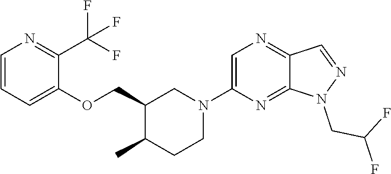 [SMILES: C[C@@H]1CCN(c2cnc3cnn(CC(F)F)c3n2)C[C@@H]1COc1cccnc1C(F)(F)F] | 48a |
| 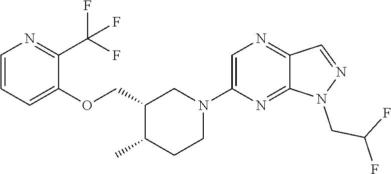 [SMILES: C[C@H]1CCN(c2cnc3cnn(CC(F)F)c3n2)C[C@H]1COc1cccnc1C(F)(F)F] | 48b |
| 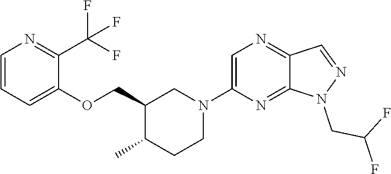 [SMILES: C[C@H]1CCN(c2cnc3cnn(CC(F)F)c3n2)C[C@@H]1COc1cccnc1C(F)(F)F] | 48c |
| 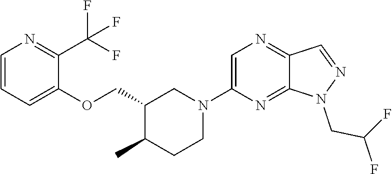 [SMILES: C[C@@H]1CCN(c2cnc3cnn(CC(F)F)c3n2)C[C@H]1COc1cccnc1C(F)(F)F] | 48d |
| 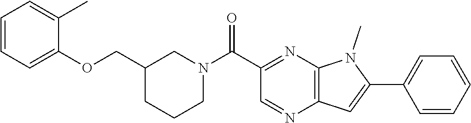 [SMILES: Cc1ccccc1OCC1CCCN(C(=O)c2cnc3cc(-c4ccccc4)n(C)c3n2)C1] | 49 |
| 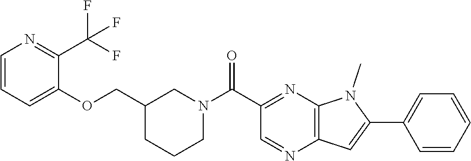 [SMILES: Cn1c(-c2ccccc2)cc2ncc(C(=O)N3CCCC(COc4cccnc4C(F)(F)F)C3)nc21] | 50 |
| 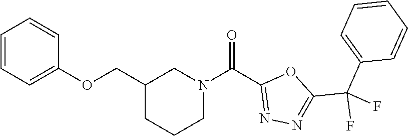 [SMILES: O=C(c1nnc(C(F)(F)c2ccccc2)o1)N1CCCC(COc2ccccc2)C1] | 51 |
| 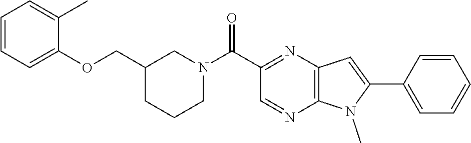 [SMILES: Cc1ccccc1OCC1CCCN(C(=O)c2cnc3c(cc(-c4ccccc4)n3C)n2)C1] | 52 |
| 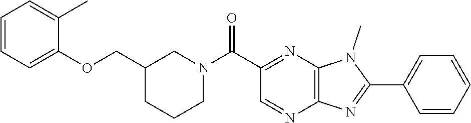 [SMILES: Cc1ccccc1OCC1CCCN(C(=O)c2cnc3nc(-c4ccccc4)n(C)c3n2)C1] | 53 |
| 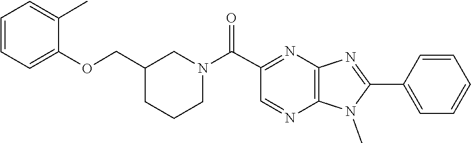 [SMILES: Cc1ccccc1OCC1CCCN(C(=O)c2cnc3c(n2)nc(-c2ccccc2)n3C)C1] | 54 |
| 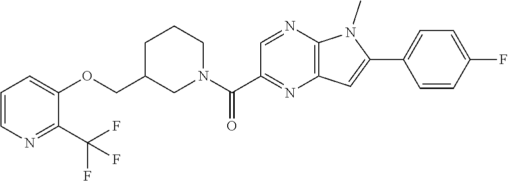 [SMILES: Cn1c(-c2ccc(F)cc2)cc2nc(C(=O)N3CCCC(COc4cccnc4C(F)(F)F)C3)cnc21] | 55 |
| 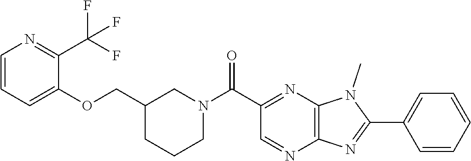 [SMILES: Cn1c(-c2ccccc2)nc2ncc(C(=O)N3CCCC(COc4cccnc4C(F)(F)F)C3)nc21] | 56 |
| 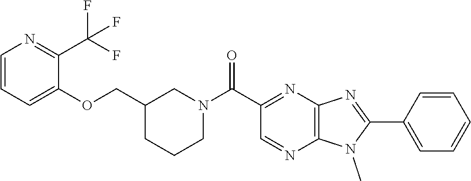 [SMILES: Cn1c(-c2ccccc2)nc2nc(C(=O)N3CCCC(COc4cccnc4C(F)(F)F)C3)cnc21] | 57 |
| 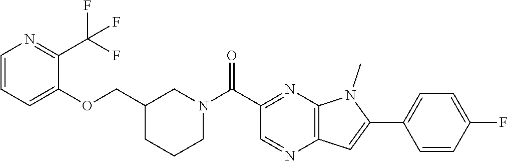 [SMILES: Cn1c(-c2ccc(F)cc2)cc2ncc(C(=O)N3CCCC(COc4cccnc4C(F)(F)F)C3)nc21] | 58 |
| 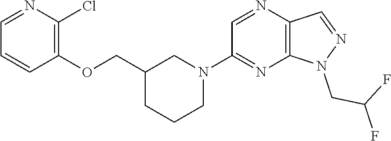 [SMILES: FC(F)Cn1ncc2ncc(N3CCCC(COc4cccnc4Cl)C3)nc21] | 59 |
| 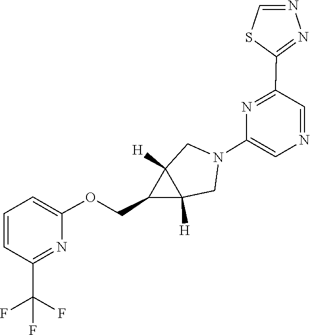 [SMILES: FC(F)(F)c1cccc(OC[C@@H]2[C@H]3CN(c4cncc(-c5nncs5)n4)C[C@@H]23)n1] | 60 |
| 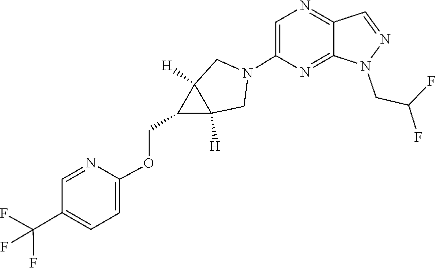 [SMILES: FC(F)Cn1ncc2ncc(N3C[C@@H]4[C@@H](COc5ccc(C(F)(F)F)cn5)[C@@H]4C3)nc21] | 61 |
| 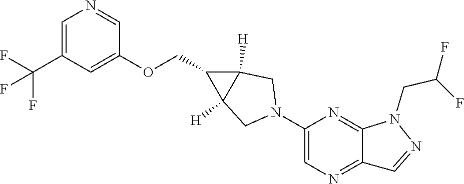 [SMILES: FC(F)Cn1ncc2ncc(N3C[C@@H]4[C@@H](COc5cncc(C(F)(F)F)c5)[C@@H]4C3)nc21] | 62 |
| 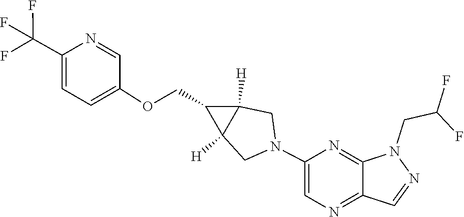 [SMILES: FC(F)Cn1ncc2ncc(N3C[C@@H]4[C@@H](COc5ccc(C(F)(F)F)nc5)[C@@H]4C3)nc21] | 63 |
| 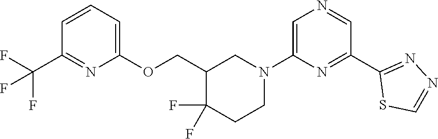 [SMILES: FC(F)(F)c1cccc(OCC2CN(c3cncc(-c4nncs4)n3)CCC2(F)F)n1] | 64 |
| 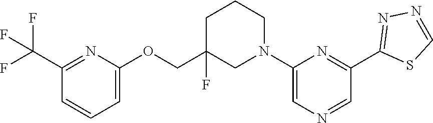 [SMILES: FC1(COc2cccc(C(F)(F)F)n2)CCCN(c2cncc(-c3nncs3)n2)C1] | 65 |
| 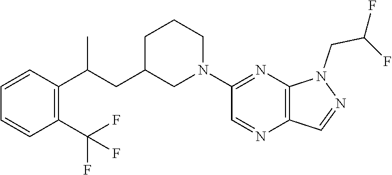 [SMILES: CC(CC1CCCN(c2cnc3cnn(CC(F)F)c3n2)C1)c1ccccc1C(F)(F)F] | 66 |
| 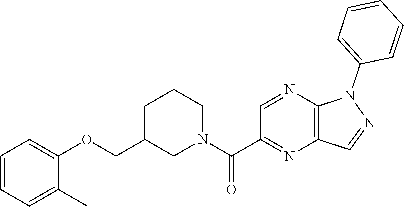 [SMILES: Cc1ccccc1OCC1CCCN(C(=O)c2cnc3c(cnn3-c3ccccc3)n2)C1] | 67 |
| 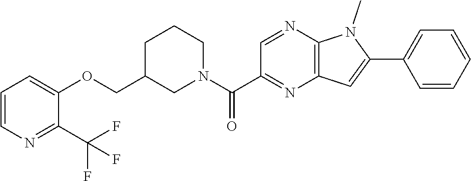 [SMILES: Cn1c(-c2ccccc2)cc2nc(C(=O)N3CCCC(COc4cccnc4C(F)(F)F)C3)cnc21] | 68 |
| 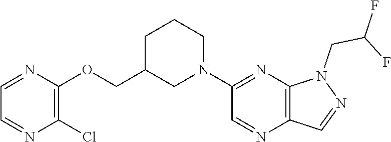 [SMILES: FC(F)Cn1ncc2ncc(N3CCCC(COc4nccnc4Cl)C3)nc21] | 69 |
| 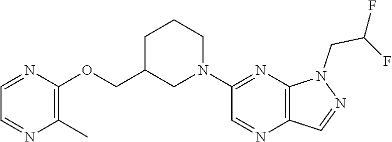 [SMILES: Cc1nccnc1OCC1CCCN(c2cnc3cnn(CC(F)F)c3n2)C1] | 70 |
| 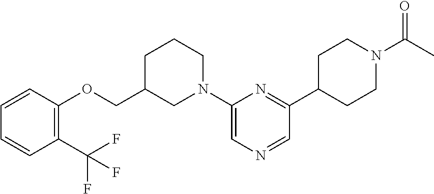 [SMILES: CC(=O)N1CCC(c2cncc(N3CCCC(COc4ccccc4C(F)(F)F)C3)n2)CC1] | 71 |
| 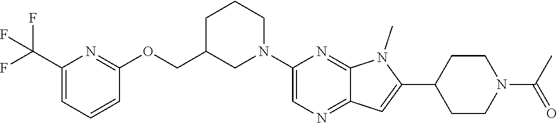 [SMILES: CC(=O)N1CCC(c2cc3ncc(N4CCCC(COc5cccc(C(F)(F)F)n5)C4)nc3n2C)CC1] | 72 |
| 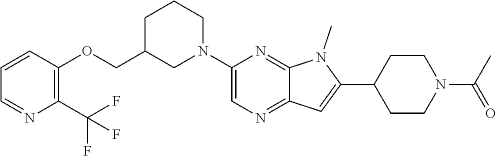 [SMILES: CC(=O)N1CCC(c2cc3ncc(N4CCCC(COc5cccnc5C(F)(F)F)C4)nc3n2C)CC1] | 73 |
| 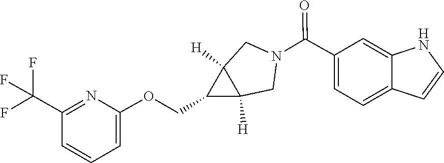 [SMILES: O=C(c1ccc2cc[nH]c2c1)N1C[C@@H]2[C@@H](COc3cccc(C(F)(F)F)n3)[C@@H]2C1] | 74 |
| 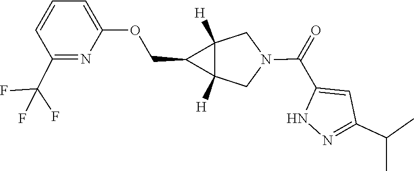 [SMILES: CC(C)c1cc(C(=O)N2C[C@@H]3[C@@H](COc4cccc(C(F)(F)F)n4)[C@@H]3C2)[nH]n1] | 75 |
| 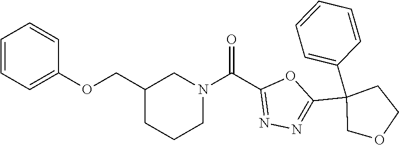 [SMILES: O=C(c1nnc(C2(c3ccccc3)CCOC2)o1)N1CCCC(COc2ccccc2)C1] | 76 |
| 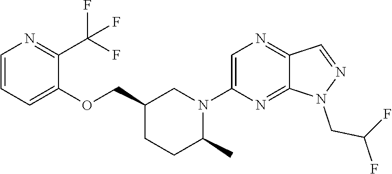 [SMILES: C[C@H]1CC[C@@H](COc2cccnc2C(F)(F)F)CN1c1cnc2cnn(CC(F)F)c2n1] | 77 |
| 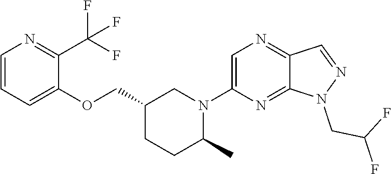 [SMILES: C[C@H]1CC[C@H](COc2cccnc2C(F)(F)F)CN1c1cnc2cnn(CC(F)F)c2n1] | 78 |
| 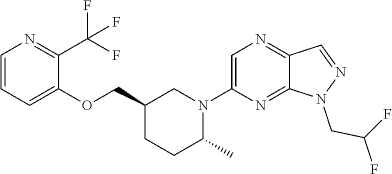 [SMILES: C[C@@H]1CC[C@@H](COc2cccnc2C(F)(F)F)CN1c1cnc2cnn(CC(F)F)c2n1] | 79 |
| 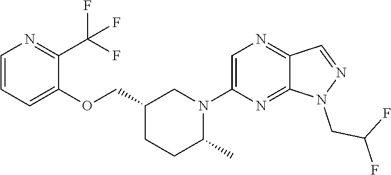 [SMILES: C[C@@H]1CC[C@H](COc2cccnc2C(F)(F)F)CN1c1cnc2cnn(CC(F)F)c2n1] | 80 |
| 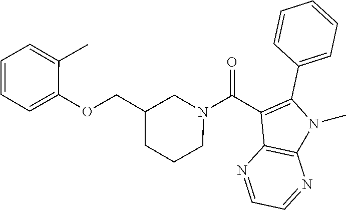 [SMILES: Cc1ccccc1OCC1CCCN(C(=O)c2c(-c3ccccc3)n(C)c3nccnc23)C1] | 81 |
| 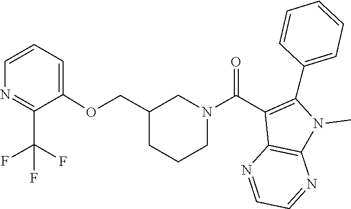 [SMILES: Cn1c(-c2ccccc2)c(C(=O)N2CCCC(COc3cccnc3C(F)(F)F)C2)c2nccnc21] | 82 |
| 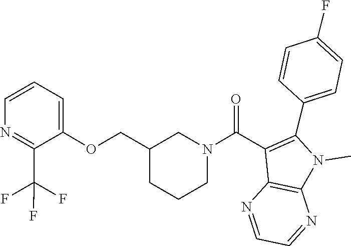 [SMILES: Cn1c(-c2ccc(F)cc2)c(C(=O)N2CCCC(COc3cccnc3C(F)(F)F)C2)c2nccnc21] | 83 |
| 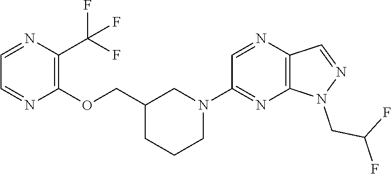 [SMILES: FC(F)Cn1ncc2ncc(N3CCCC(COc4nccnc4C(F)(F)F)C3)nc21] | 84 |
| 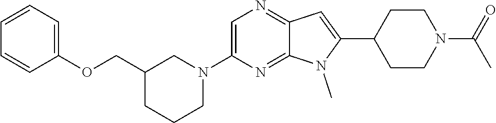 [SMILES: CC(=O)N1CCC(c2cc3ncc(N4CCCC(COc5ccccc5)C4)nc3n2C)CC1] | 85 |
| 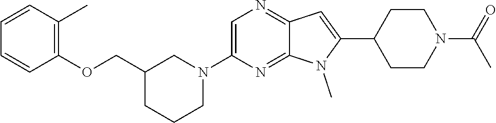 [SMILES: CC(=O)N1CCC(c2cc3ncc(N4CCCC(COc5ccccc5C)C4)nc3n2C)CC1] | 86 |
| 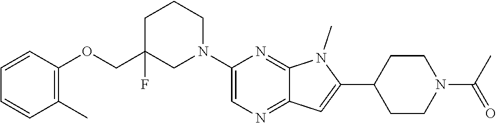 [SMILES: CC(=O)N1CCC(c2cc3ncc(N4CCCC(F)(COc5ccccc5C)C4)nc3n2C)CC1] | 87 |
| 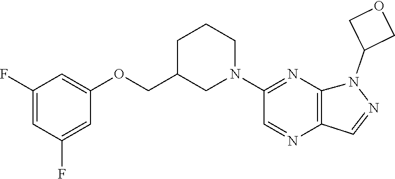 | 88 |
| 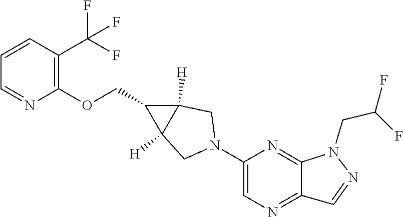 | 89 |
| 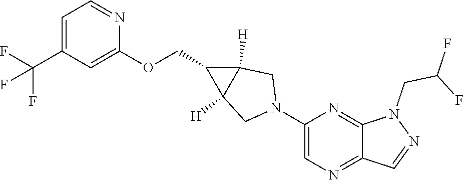 [SMILES: FC(F)Cn1ncc2ncc(N3C[C@@H]4[C@@H](COc5cc(C(F)(F)F)ccn5)[C@@H]4C3)nc21] | 90 |
| 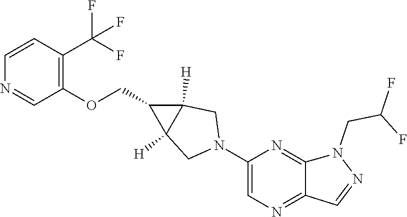 | 91 |
| 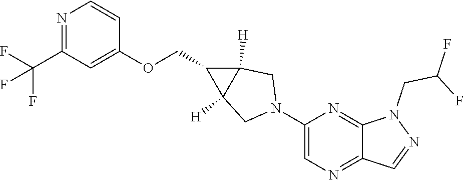 [SMILES: FC(F)Cn1ncc2ncc(N3C[C@@H]4[C@@H](COc5ccnc(C(F)(F)F)c5)[C@@H]4C3)nc21] | 92 |
| 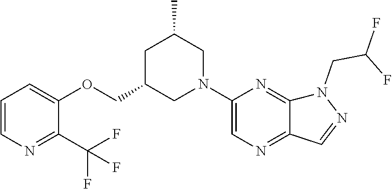 | 93 |
| 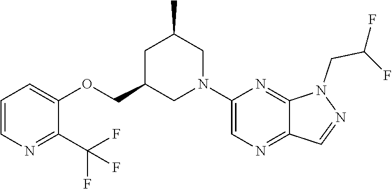 [SMILES: C[C@@H]1C[C@H](COc2cccnc2C(F)(F)F)CN(c2cnc3cnn(CC(F)F)c3n2)C1] | 94 |
| 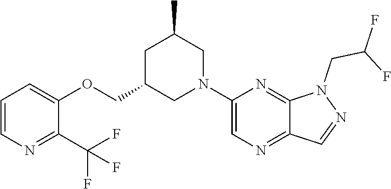 | 95 |
| 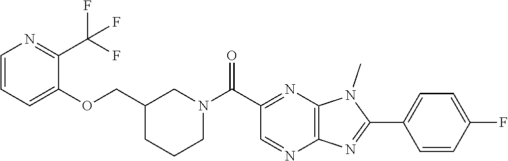 [SMILES: Cn1c(-c2ccc(F)cc2)nc2ncc(C(=O)N3CCCC(COc4cccnc4C(F)(F)F)C3)nc21] | 96 |
| 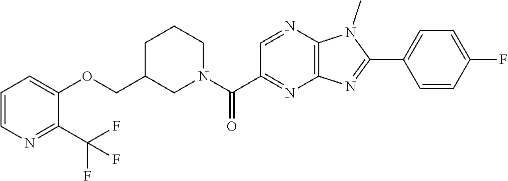 [SMILES: Cn1c(-c2ccc(F)cc2)nc2nc(C(=O)N3CCCC(COc4cccnc4C(F)(F)F)C3)cnc21] | 97 |
| 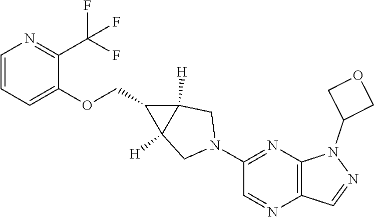 [SMILES: FC(F)(F)c1ncccc1OC[C@@H]1[C@H]2CN(c3cnc4cnn(C5COC5)c4n3)C[C@@H]12] | 98 |
| 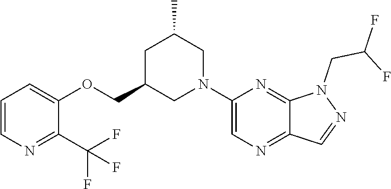 | 100 |
| 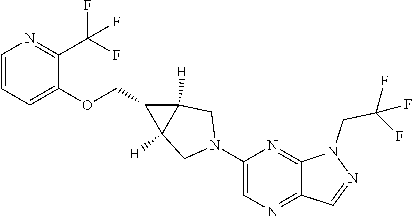 [SMILES: FC(F)(F)Cn1ncc2ncc(N3C[C@@H]4[C@@H](COc5cccnc5C(F)(F)F)[C@@H]4C3)nc21] | 101 |
| 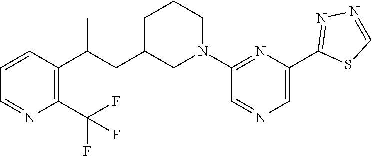 [SMILES: CC(CC1CCCN(c2cncc(-c3nncs3)n2)C1)c1cccnc1C(F)(F)F] | 102 |
| 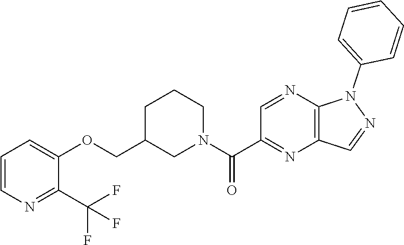 [SMILES: O=C(c1cnc2c(cnn2-c2ccccc2)n1)N1CCCC(COc2cccnc2C(F)(F)F)C1] | 103 |
| 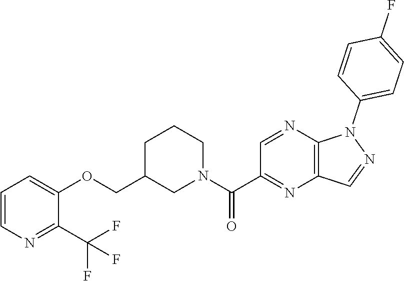 [SMILES: O=C(c1cnc2c(cnn2-c2ccc(F)cc2)n1)N1CCCC(COc2cccnc2C(F)(F)F)C1] | 104 |
| 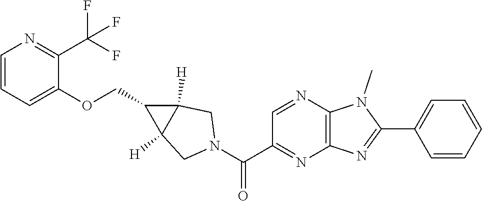 [SMILES: Cn1c(-c2ccccc2)nc2nc(C(=O)N3C[C@@H]4[C@@H](COc5cccnc5C(F)(F)F)[C@@H]4C3)cnc21] | 105 |
| 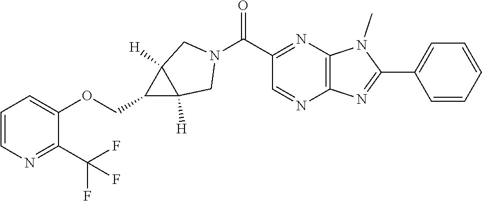 [SMILES: Cn1c(-c2ccccc2)nc2ncc(C(=O)N3C[C@@H]4[C@@H](COc5cccnc5C(F)(F)F)[C@@H]4C3)nc21] | 106 |
| 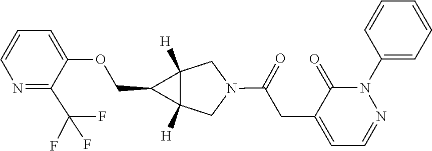 | 107 |
| 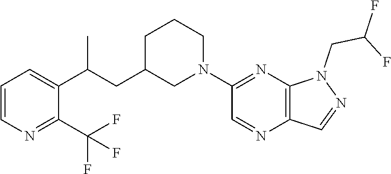 [SMILES: CC(CC1CCCN(c2cnc3cnn(CC(F)F)c3n2)C1)c1cccnc1C(F)(F)F] | 108 |
| 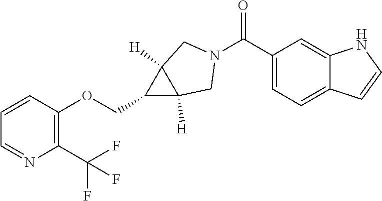 [SMILES: O=C(c1ccc2cc[nH]c2c1)N1C[C@@H]2[C@@H](COc3cccnc3C(F)(F)F)[C@@H]2C1] | 109 |
| 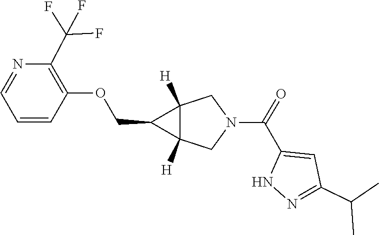 [SMILES: CC(C)c1cc(C(=O)N2C[C@@H]3[C@@H](COc4cccnc4C(F)(F)F)[C@@H]3C2)[nH]n1] | 110 |
| 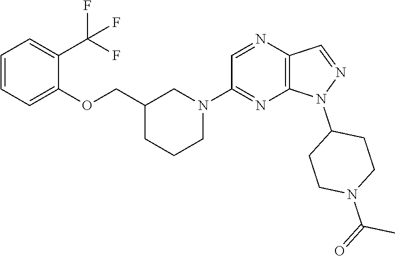 [SMILES: CC(=O)N1CCC(n2ncc3ncc(N4CCCC(COc5ccccc5C(F)(F)F)C4)nc32)CC1] | 111 |
| 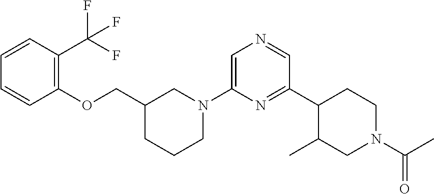 [SMILES: CC(=O)N1CCC(c2cncc(N3CCCC(COc4ccccc4C(F)(F)F)C3)n2)C(C)C1] | 112 |
| 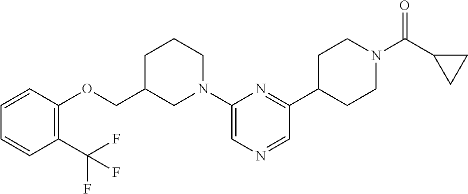 [SMILES: O=C(C1CC1)N1CCC(c2cncc(N3CCCC(COc4ccccc4C(F)(F)F)C3)n2)CC1] | 113 |
| 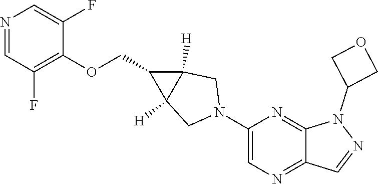 | 114 |
| 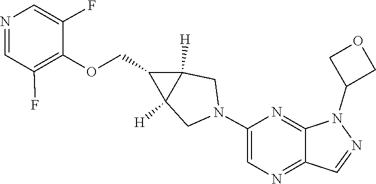 [SMILES: Fc1cncc(F)c1OC[C@@H]1[C@H]2CN(c3cnc4cnn(C5COC5)c4n3)C[C@@H]12] | 115 |
| 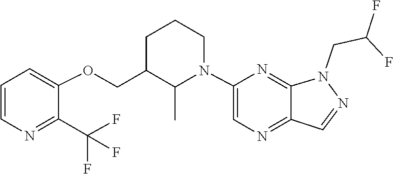 | 116 |
| 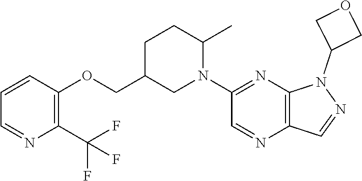 [SMILES: CC1CCC(COc2cccnc2C(F)(F)F)CN1c1cnc2cnn(C3COC3)c2n1] | 117 |
| 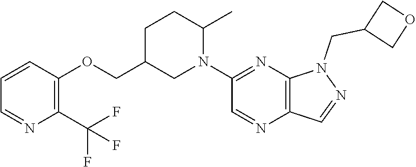 [SMILES: CC1CCC(COc2cccnc2C(F)(F)F)CN1c1cnc2cnn(CC3COC3)c2n1] | 118 |
| 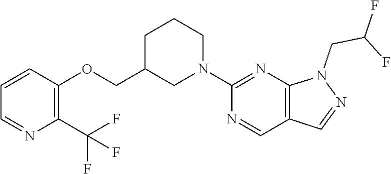 | 119 |
| 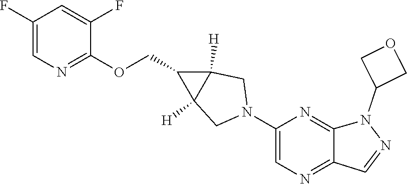 | 120 |
| 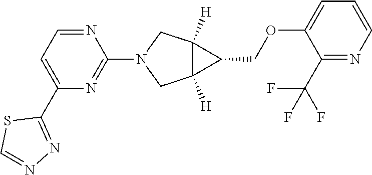 [SMILES: FC(F)(F)c1ncccc1OC[C@@H]1[C@H]2CN(c3nccc(-c4nncs4)n3)C[C@@H]12] | 121 |
| 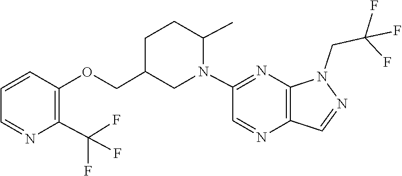 [SMILES: CC1CCC(COc2cccnc2C(F)(F)F)CN1c1cnc2cnn(CC(F)(F)F)c2n1] | 122 |
| 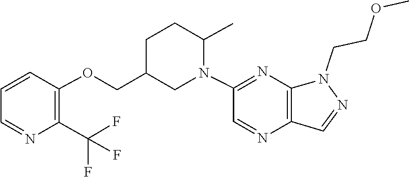 [SMILES: COCCn1ncc2ncc(N3CC(COc4cccnc4C(F)(F)F)CCC3C)nc21] | 123 |
| 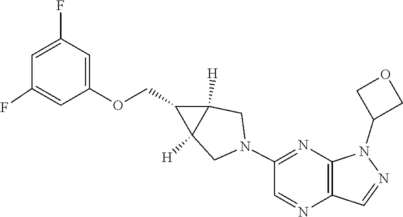 [SMILES: Fc1cc(F)cc(OC[C@@H]2[C@H]3CN(c4cnc5cnn(C6COC6)c5n4)C[C@@H]23)c1] | 124 |
| 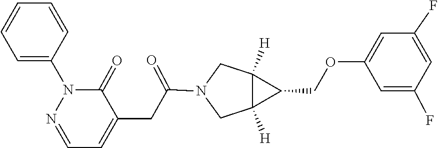 [SMILES: O=C(Cc1ccnn(-c2ccccc2)c1=O)N1C[C@@H]2[C@@H](COc3cc(F)cc(F)c3)[C@@H]2C1] | 125 |
| 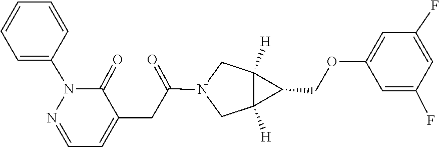 [SMILES: O=C(Cc1ccnn(-c2ccccc2)c1=O)N1C[C@@H]2[C@@H](COc3cc(F)cc(F)c3)[C@@H]2C1] | 126 |
| 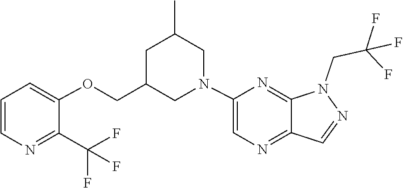 [SMILES: CC1CC(COc2cccnc2C(F)(F)F)CN(c2cnc3cnn(CC(F)(F)F)c3n2)C1] | 127 |
| 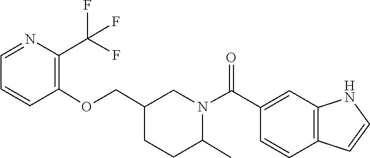 [SMILES: CC1CCC(COc2cccnc2C(F)(F)F)CN1C(=O)c1ccc2cc[nH]c2c1] | 128 |
| 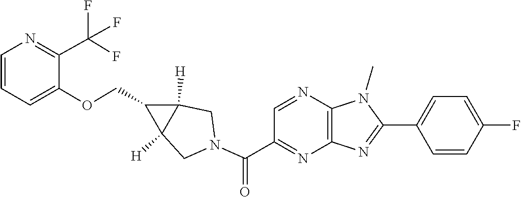 [SMILES: Cn1c(-c2ccc(F)cc2)nc2nc(C(=O)N3C[C@@H]4[C@@H](COc5cccnc5C(F)(F)F)[C@@H]4C3)cnc21] | 223 |
| 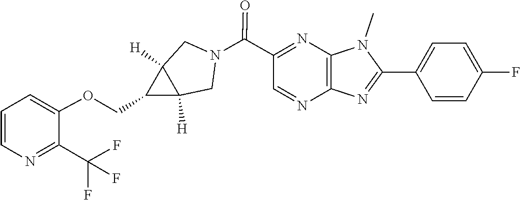 [SMILES: Cn1c(-c2ccc(F)cc2)nc2ncc(C(=O)N3C[C@@H]4[C@@H](COc5cccnc5C(F)(F)F)[C@@H]4C3)nc21] | 224 |
| 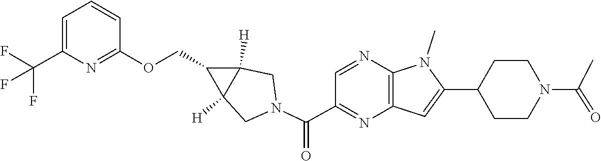 [SMILES: CC(=O)N1CCC(c2cc3nc(C(=O)N4C[C@@H]5[C@@H](COc6cccc(C(F)(F)F)n6)[C@@H]5C4)cnc3n2C)CC1] | 225 |
| 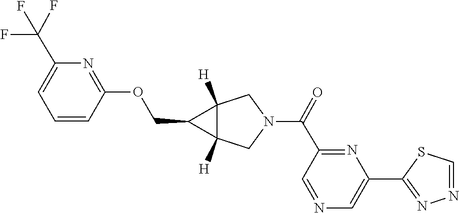 [SMILES: O=C(c1cncc(-c2nncs2)n1)N1C[C@@H]2[C@@H](COc3cccc(C(F)(F)F)n3)[C@@H]2C1] | 226 |
| 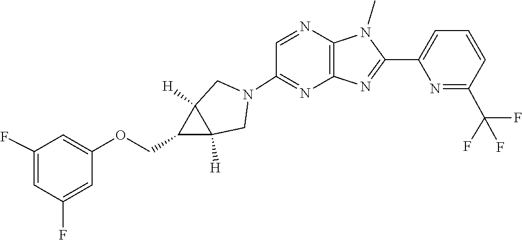 [SMILES: Cn1c(-c2cccc(C(F)(F)F)n2)nc2nc(N3C[C@@H]4[C@@H](COc5cc(F)cc(F)c5)[C@@H]4C3)cnc21] | 227 |
| 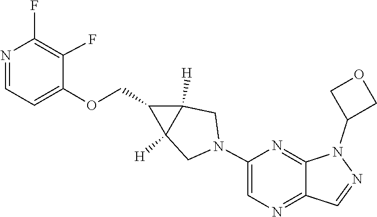 [SMILES: Fc1nccc(OC[C@@H]2[C@H]3CN(c4cnc5cnn(C6COC6)c5n4)C[C@@H]23)c1F] | 228 |
| 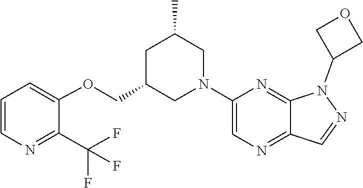 [SMILES: C[C@H]1C[C@@H](COc2cccnc2C(F)(F)F)CN(c2cnc3cnn(C4COC4)c3n2)C1] | 229a |
| 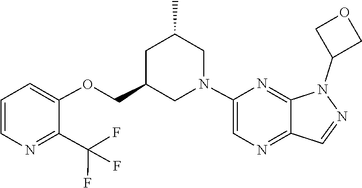 [SMILES: C[C@H]1C[C@H](COc2cccnc2C(F)(F)F)CN(c2cnc3cnn(C4COC4)c3n2)C1] | 229b |
| 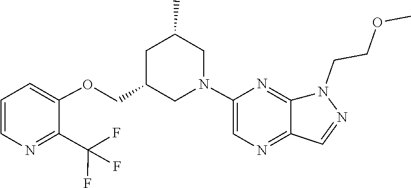 [SMILES: COCCn1ncc2ncc(N3C[C@@H](C)C[C@@H](COc4cccnc4C(F)(F)F)C3)nc21] | 230a |
| 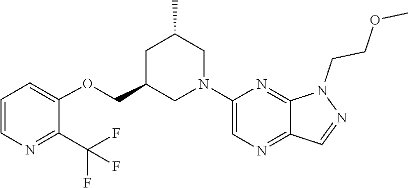 [SMILES: COCCn1ncc2ncc(N3C[C@@H](C)C[C@H](COc4cccnc4C(F)(F)F)C3)nc21] | 230b |
| 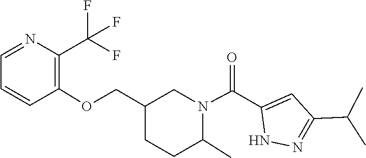 [SMILES: CC(C)c1cc(C(=O)N2CC(COc3cccnc3C(F)(F)F)CCC2C)[nH]n1] | 231 |
| 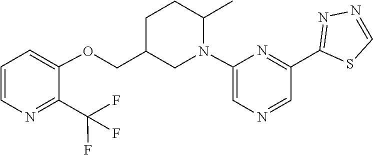 [SMILES: CC1CCC(COc2cccnc2C(F)(F)F)CN1c1cncc(-c2nncs2)n1] | 232 |
| 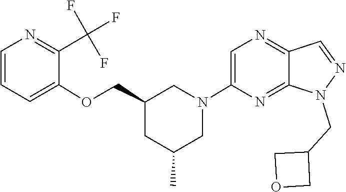 [SMILES: C[C@@H]1C[C@@H](COc2cccnc2C(F)(F)F)CN(c2cnc3cnn(CC4COC4)c3n2)C1] | 233a |
| 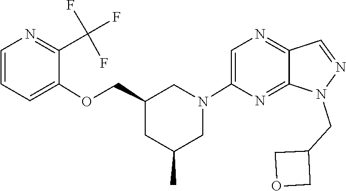 [SMILES: C[C@H]1C[C@@H](COc2cccnc2C(F)(F)F)CN(c2cnc3cnn(CC4COC4)c3n2)C1] | 233b |
| 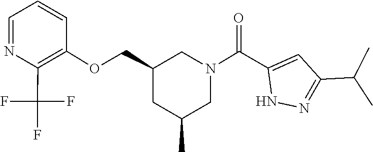 [SMILES: CC(C)c1cc(C(=O)N2C[C@@H](C)C[C@@H](COc3cccnc3C(F)(F)F)C2)[nH]n1] | 234a |
| 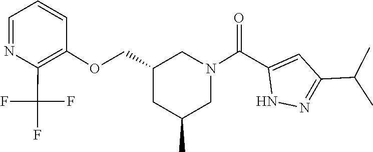 [SMILES: CC(C)c1cc(C(=O)N2C[C@@H](C)C[C@H](COc3cccnc3C(F)(F)F)C2)[nH]n1] | 234b |
| 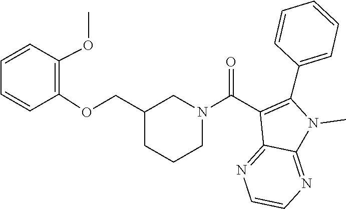 [SMILES: COc1ccccc1OCC1CCCN(C(=O)c2c(-c3ccccc3)n(C)c3nccnc23)C1] | 235 |
| 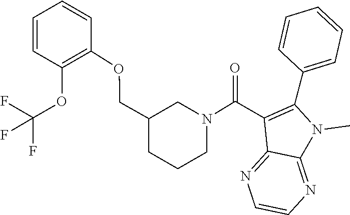 [SMILES: Cn1c(-c2ccccc2)c(C(=O)N2CCCC(COc3ccccc3OC(F)(F)F)C2)c2nccnc21] | 236 |
| 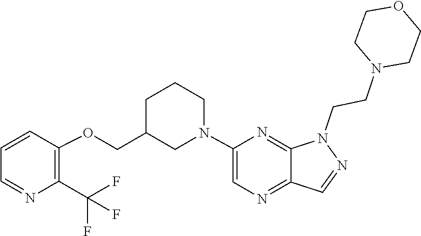 [SMILES: FC(F)(F)c1ncccc1OCC1CCCN(c2cnc3cnn(CCN4CCOCC4)c3n2)C1] | 237 |
| 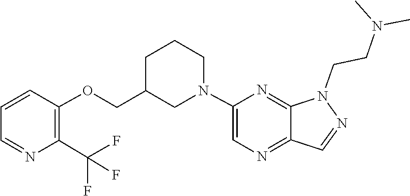 [SMILES: CN(C)CCn1ncc2ncc(N3CCCC(COc4cccnc4C(F)(F)F)C3)nc21] | 238 |
| 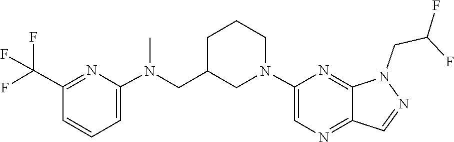 [SMILES: CN(CC1CCCN(c2cnc3cnn(CC(F)F)c3n2)C1)c1cccc(C(F)(F)F)n1] | 239 |
| 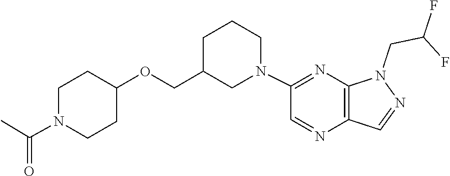 [SMILES: CC(=O)N1CCC(OCC2CCCN(c3cnc4cnn(CC(F)F)c4n3)C2)CC1] | 240 |
| 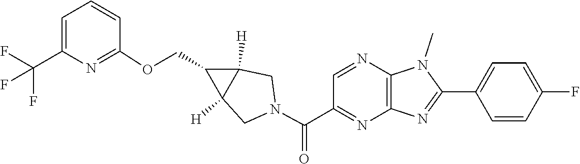 [SMILES: Cn1c(-c2ccc(F)cc2)nc2nc(C(=O)N3C[C@@H]4[C@@H](COc5cccc(C(F)(F)F)n5)[C@@H]4C3)cnc21] | 241 |
| 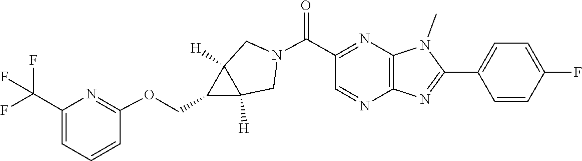 [SMILES: Cn1c(-c2ccc(F)cc2)nc2ncc(C(=O)N3C[C@@H]4[C@@H](COc5cccc(C(F)(F)F)n5)[C@@H]4C3)nc21] | 242 |
| 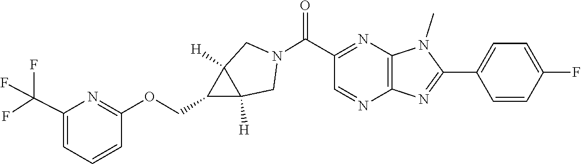 [SMILES: Cn1c(-c2ccc(F)cc2)nc2ncc(C(=O)N3C[C@@H]4[C@@H](COc5cccc(C(F)(F)F)n5)[C@@H]4C3)nc21] | 243 |
| 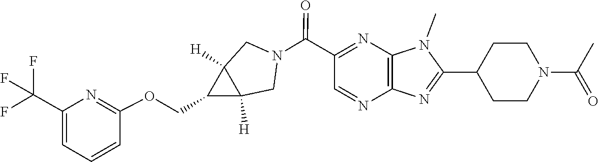 [SMILES: CC(=O)N1CCC(c2nc3ncc(C(=O)N4C[C@@H]5[C@@H](COc6cccc(C(F)(F)F)n6)[C@@H]5C4)nc3n2C)CC1] | 244 |
| 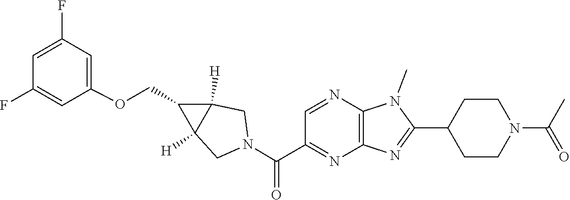 [SMILES: CC(=O)N1CCC(c2nc3nc(C(=O)N4C[C@@H]5[C@@H](COc6cc(F)cc(F)c6)[C@@H]5C4)cnc3n2C)CC1] | 245 |
| 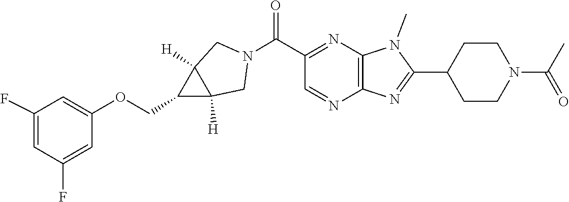 [SMILES: CC(=O)N1CCC(c2nc3ncc(C(=O)N4C[C@@H]5[C@@H](COc6cc(F)cc(F)c6)[C@@H]5C4)nc3n2C)CC1] | 246 |
| 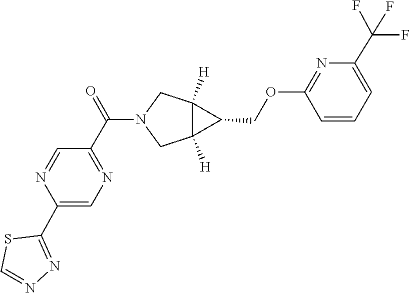 [SMILES: O=C(c1cnc(-c2nncs2)cn1)N1C[C@@H]2[C@@H](COc3cccc(C(F)(F)F)n3)[C@@H]2C1] | 247 |
| 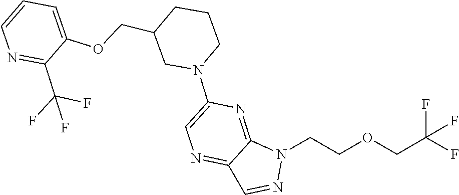 [SMILES: FC(F)(F)COCCn1ncc2ncc(N3CCCC(COc4cccnc4C(F)(F)F)C3)nc21] | 248 |
| 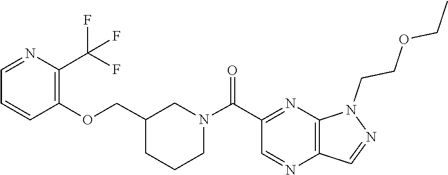 [SMILES: CCOCCn1ncc2ncc(C(=O)N3CCCC(COc4cccnc4C(F)(F)F)C3)nc21] | 249 |
| 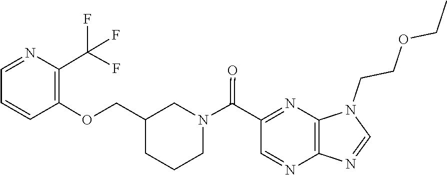 [SMILES: CCOCCn1cnc2ncc(C(=O)N3CCCC(COc4cccnc4C(F)(F)F)C3)nc21] | 250 |
| 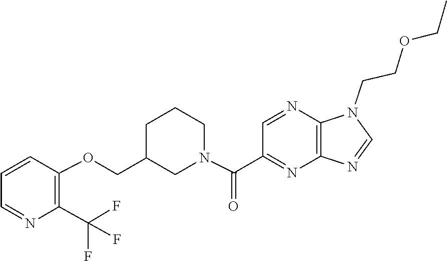 [SMILES: CCOCCn1cnc2nc(C(=O)N3CCCC(COc4cccnc4C(F)(F)F)C3)cnc21] | 251 |
| 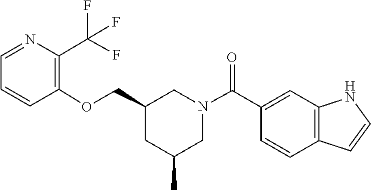 [SMILES: C[C@H]1C[C@@H](COc2cccnc2C(F)(F)F)CN(C(=O)c2ccc3cc[nH]c3c2)C1] | 252a |
| 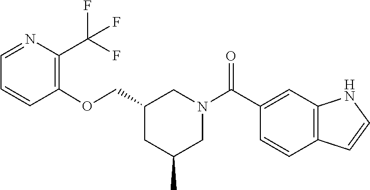 [SMILES: C[C@H]1C[C@H](COc2cccnc2C(F)(F)F)CN(C(=O)c2ccc3cc[nH]c3c2)C1] | 252b |
| 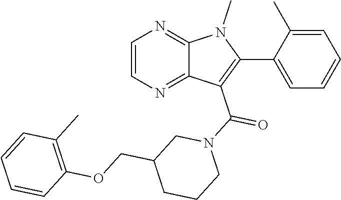 [SMILES: Cc1ccccc1OCC1CCCN(C(=O)c2c(-c3ccccc3C)n(C)c3nccnc23)C1] | 253 |
| 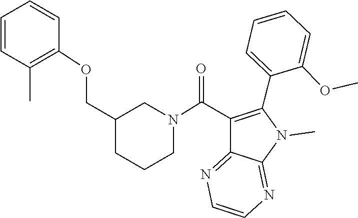 [SMILES: COc1ccccc1-c1c(C(=O)N2CCCC(COc3ccccc3C)C2)c2nccnc2n1C] | 254 |
| 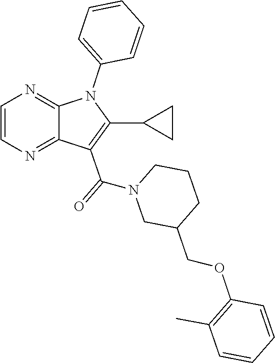 [SMILES: Cc1ccccc1OCC1CCCN(C(=O)c2c(C3CC3)n(-c3ccccc3)c3nccnc23)C1] | 255 |
| 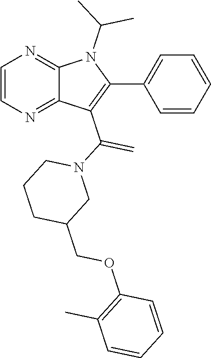 [SMILES: C=C(c1c(-c2ccccc2)n(C(C)C)c2nccnc12)N1CCCC(COc2ccccc2C)C1] | 256 |
| 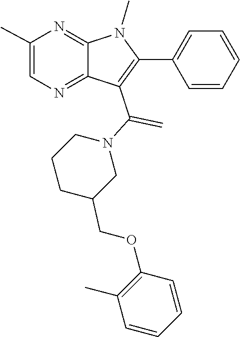 [SMILES: C=C(c1c(-c2ccccc2)n(C)c2nc(C)cnc12)N1CCCC(COc2ccccc2C)C1] | 257 |
| 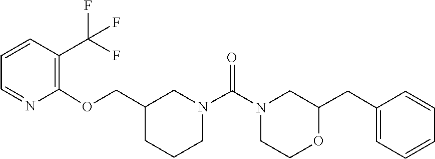 [SMILES: O=C(N1CCCC(COc2ncccc2C(F)(F)F)C1)N1CCOC(Cc2ccccc2)C1] | 258 |
| 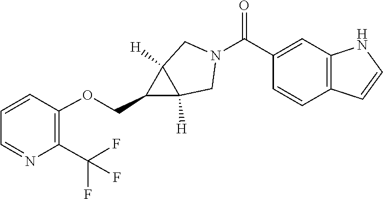 [SMILES: O=C(c1ccc2cc[nH]c2c1)N1C[C@@H]2[C@H](COc3cccnc3C(F)(F)F)[C@@H]2C1] | 259 |
| 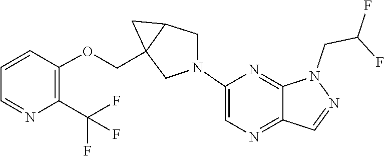 [SMILES: FC(F)Cn1ncc2ncc(N3CC4CC4(COc4cccnc4C(F)(F)F)C3)nc21] | 260 |
| 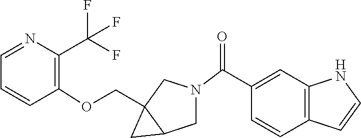 [SMILES: O=C(c1ccc2cc[nH]c2c1)N1CC2CC2(COc2cccnc2C(F)(F)F)C1] | 261 |
| 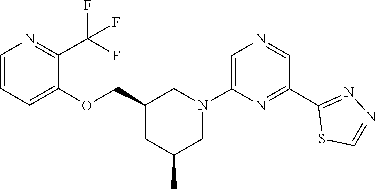 [SMILES: C[C@H]1C[C@@H](COc2cccnc2C(F)(F)F)CN(c2cncc(-c3nncs3)n2)C1] | 262a |
| 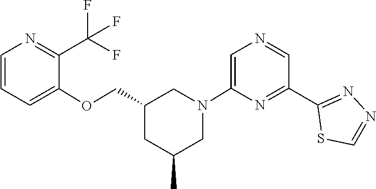 [SMILES: C[C@H]1C[C@H](COc2cccnc2C(F)(F)F)CN(c2cncc(-c3nncs3)n2)C1] | 262b |
| 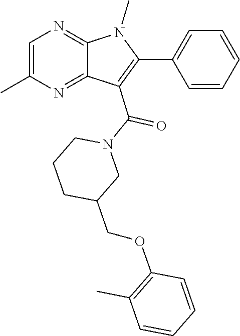 [SMILES: Cc1cnc2c(n1)c(C(=O)N1CCCC(COc3ccccc3C)C1)c(-c1ccccc1)n2C] | 263 |
| 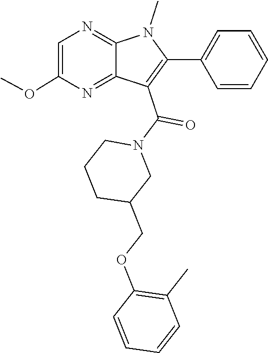 [SMILES: COc1cnc2c(n1)c(C(=O)N1CCCC(COc3ccccc3C)C1)c(-c1ccccc1)n2C] | 264 |
| 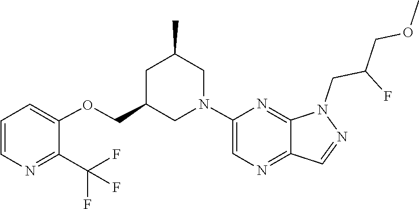 [SMILES: COCC(F)Cn1ncc2ncc(N3C[C@H](C)C[C@H](COc4cccnc4C(F)(F)F)C3)nc21] | 265 |
| 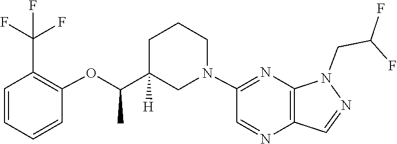 [SMILES: C[C@@H](Oc1ccccc1C(F)(F)F)[C@H]1CCCN(c2cnc3cnn(CC(F)F)c3n2)C1] | 266a |
| 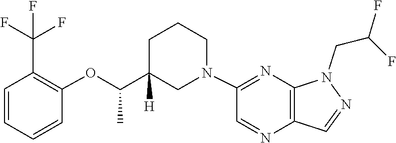 [SMILES: C[C@H](Oc1ccccc1C(F)(F)F)[C@@H]1CCCN(c2cnc3cnn(CC(F)F)c3n2)C1] | 266b |
| 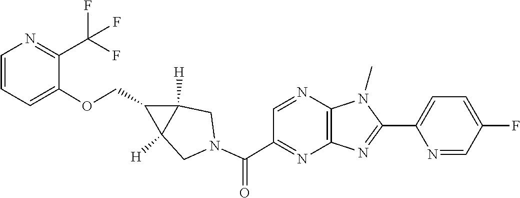 [SMILES: Cn1c(-c2ccc(F)cn2)nc2nc(C(=O)N3C[C@@H]4[C@@H](COc5cccnc5C(F)(F)F)[C@@H]4C3)cnc21] | 267 |
| 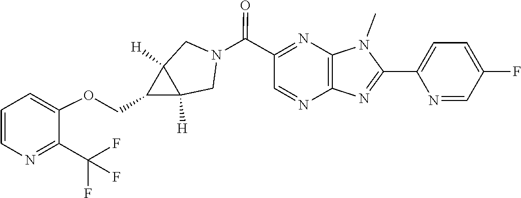 [SMILES: Cn1c(-c2ccc(F)cn2)nc2ncc(C(=O)N3C[C@@H]4[C@@H](COc5cccnc5C(F)(F)F)[C@@H]4C3)nc21] | 268 |
| 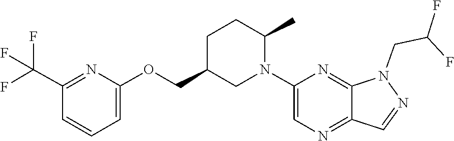 [SMILES: C[C@@H]1CC[C@H](COc2cccc(C(F)(F)F)n2)CN1c1cnc2cnn(CC(F)F)c2n1] | 269 |
| 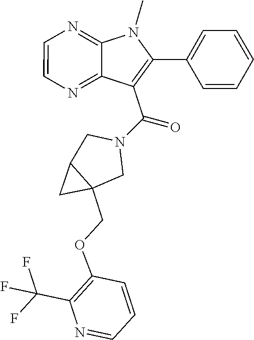 [SMILES: Cn1c(-c2ccccc2)c(C(=O)N2CC3CC3(COc3cccnc3C(F)(F)F)C2)c2nccnc21] | 270 |
| 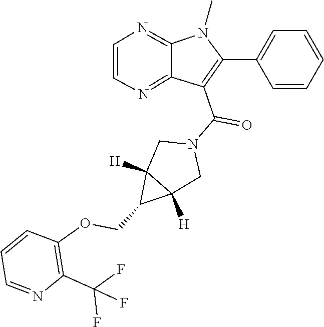 [SMILES: Cn1c(-c2ccccc2)c(C(=O)N2C[C@@H]3[C@H](COc4cccnc4C(F)(F)F)[C@@H]3C2)c2nccnc21] | 271 |
| 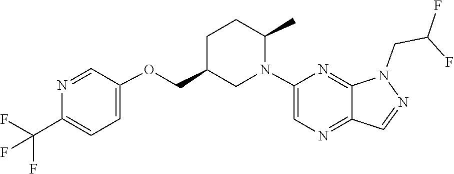 [SMILES: C[C@@H]1CC[C@H](COc2ccc(C(F)(F)F)nc2)CN1c1cnc2cnn(CC(F)F)c2n1] | 272 |
| 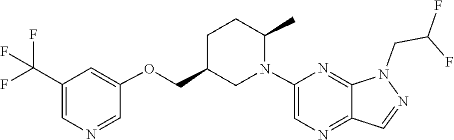 [SMILES: C[C@@H]1CC[C@H](COc2cncc(C(F)(F)F)c2)CN1c1cnc2cnn(CC(F)F)c2n1] | 273 |
| 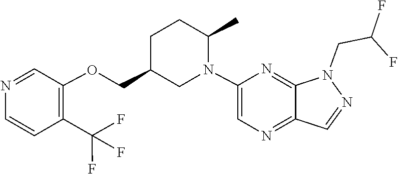 [SMILES: C[C@@H]1CC[C@H](COc2cnccc2C(F)(F)F)CN1c1cnc2cnn(CC(F)F)c2n1] | 274 |
| 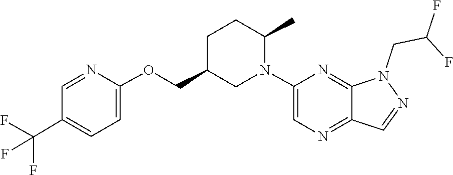 [SMILES: C[C@@H]1CC[C@H](COc2ccc(C(F)(F)F)cn2)CN1c1cnc2cnn(CC(F)F)c2n1] | 275 |
| 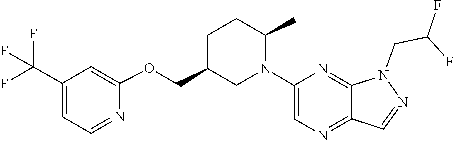 [SMILES: C[C@@H]1CC[C@H](COc2cc(C(F)(F)F)ccn2)CN1c1cnc2cnn(CC(F)F)c2n1] | 276 |
| 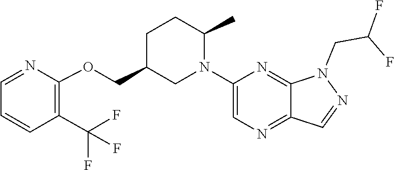 [SMILES: C[C@@H]1CC[C@H](COc2ncccc2C(F)(F)F)CN1c1cnc2cnn(CC(F)F)c2n1] | 277 |
| 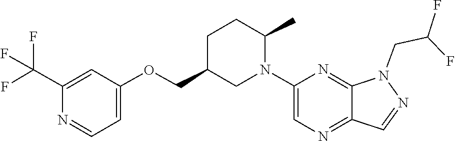 [SMILES: C[C@@H]1CC[C@H](COc2ccnc(C(F)(F)F)c2)CN1c1cnc2cnn(CC(F)F)c2n1] | 278 |
| 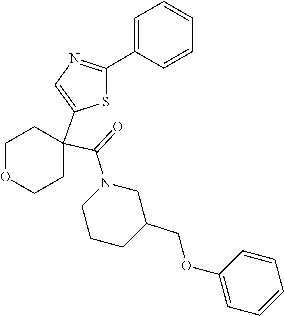 [SMILES: O=C(N1CCCC(COc2ccccc2)C1)C1(c2cnc(-c3ccccc3)s2)CCOCC1] | 279 |
| 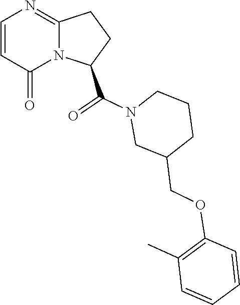 [SMILES: Cc1ccccc1OCC1CCCN(C(=O)[C@@H]2CCc3nccc(=O)n32)C1] | 280 |
| 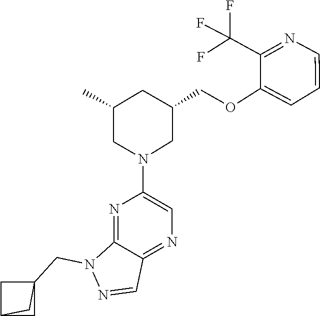 [SMILES: C[C@@H]1C[C@H](COc2cccnc2C(F)(F)F)CN(c2cnc3cnn(CC45CC(C4)C5)c3n2)C1] | 281 |
| 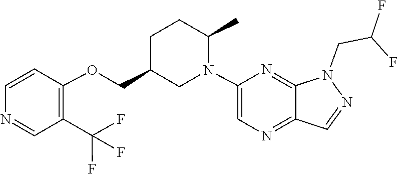 [SMILES: C[C@@H]1CC[C@H](COc2ccncc2C(F)(F)F)CN1c1cnc2cnn(CC(F)F)c2n1] | 282 |
| 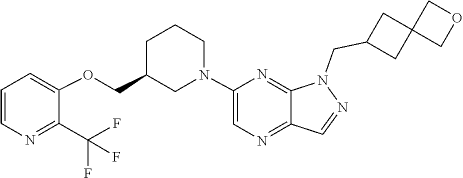 [SMILES: FC(F)(F)c1ncccc1OC[C@H]1CCCN(c2cnc3cnn(CC4CC5(COC5)C4)c3n2)C1] | 283 |
| 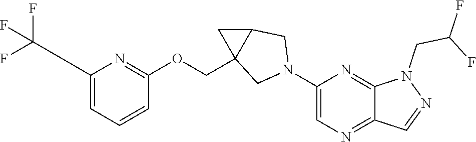 [SMILES: FC(F)Cn1ncc2ncc(N3CC4CC4(COc4cccc(CC(F)(F)F)n4)C3)nc21] | 284 |
| 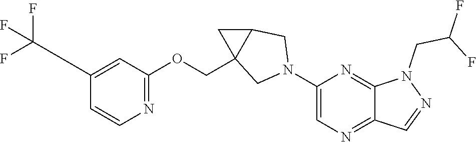 [SMILES: FC(F)Cn1ncc2ncc(N3CC4CC4(COc4cc(CC(F)(F)F)ccn4)C3)nc21] | 285 |
| 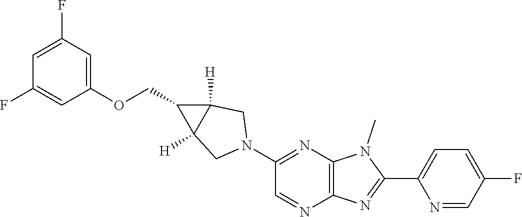 [SMILES: Cn1c(-c2ccc(F)cn2)nc2ncc(N3C[C@@H]4[C@@H](COc5cc(F)cc(F)c5)[C@@H]4C3)nc21] | 286 |
| 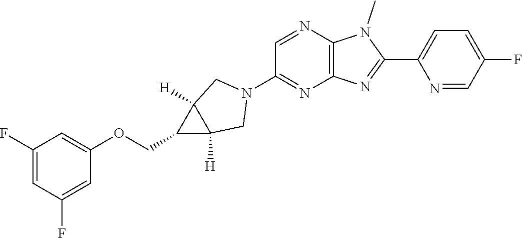 [SMILES: Cn1c(-c2ccc(F)cn2)nc2nc(N3C[C@@H]4[C@@H](COc5cc(F)cc(F)c5)[C@@H]4C3)cnc21] | 287 |
| 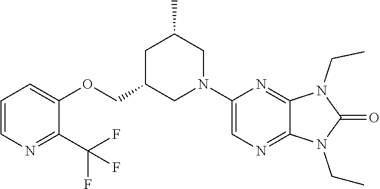 [SMILES: CCn1c(=O)n(CC)c2nc(N3C[C@@H](C)C[C@@H](COc4cccnc4C(F)(F)F)C3)cnc21] | 288a |
| 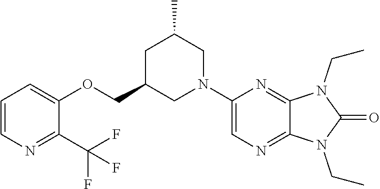 [SMILES: CCn1c(=O)n(CC)c2nc(N3C[C@@H](C)C[C@H](COc4cccnc4C(F)(F)F)C3)cnc21] | 288b |
| 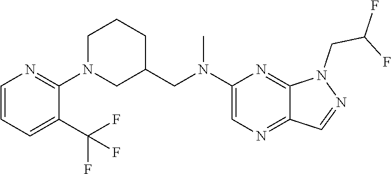 [SMILES: CN(CC1CCCN(c2ncccc2C(F)(F)F)C1)c1cnc2cnn(CC(F)F)c2n1] | 289 |
| 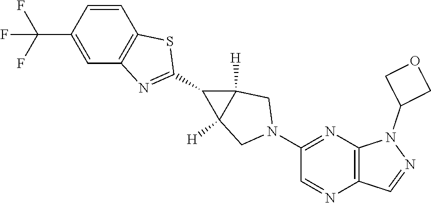 [SMILES: FC(F)(F)c1ccc2sc([C@H]3[C@@H]4CN(c5cnc6cnn(C7COC7)c6n5)C[C@@H]43)nc2c1] | 290 |
| 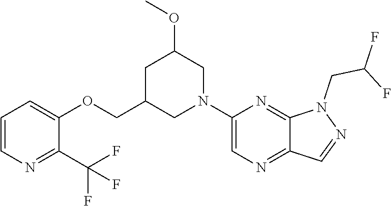 [SMILES: COC1CC(COc2cccnc2C(F)(F)F)CN(c2cnc3cnn(CC(F)F)c3n2)C1] | 291 |
| 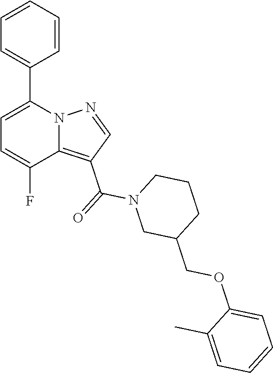 [SMILES: Cc1ccccc1OCC1CCCN(C(=O)c2cnn3c(-c4ccccc4)ccc(F)c23)C1] | 292 |
| 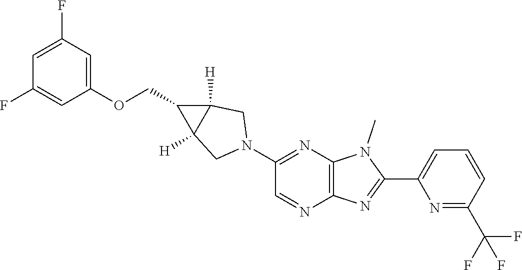 [SMILES: Cn1c(-c2cccc(C(F)(F)F)n2)nc2ncc(N3C[C@@H]4[C@@H](COc5cc(F)cc(F)c5)[C@@H]4C3)nc21] | 293 |
| 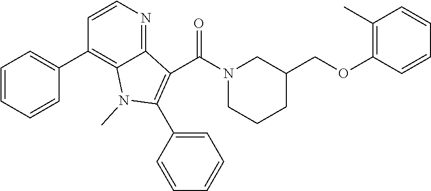 [SMILES: Cc1ccccc1OCC1CCCN(C(=O)c2c(-c3ccccc3)n(C)c3c(-c4ccccc4)ccnc23)C1] | 294 |
| 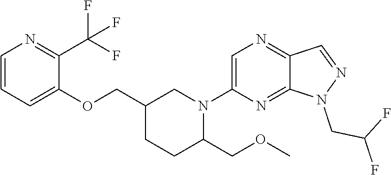 [SMILES: COCC1CCC(COc2cccnc2C(F)(F)F)CN1c1cnc2cnn(CC(F)F)c2n1] | 295 |
| 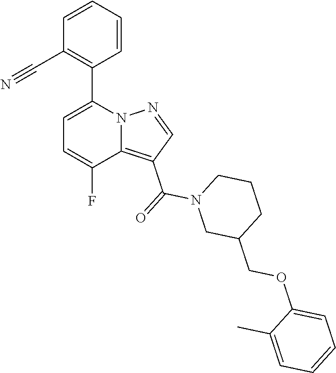 [SMILES: Cc1ccccc1OCC1CCCN(C(=O)c2cnn3c(-c4ccccc4C#N)ccc(F)c23)C1] | 296 |
| 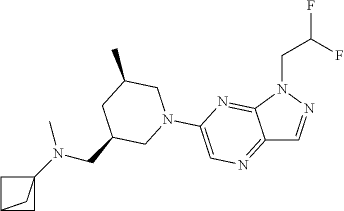 [SMILES: C[C@@H]1C[C@H](CN(C)C23CC(C2)C3)CN(c2cnc3cnn(CC(F)F)c3n2)C1] | 297 |
| 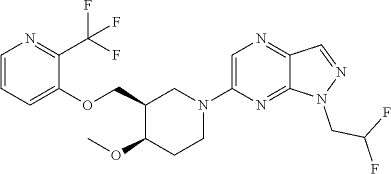 [SMILES: CO[C@@H]1CCN(c2cnc3cnn(CC(F)F)c3n2)C[C@@H]1COc1cccnc1C(F)(F)F] | 298a |
| 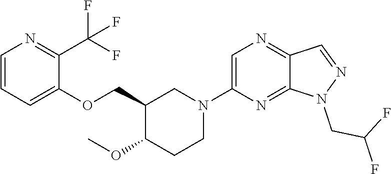 [SMILES: CO[C@H]1CCN(c2cnc3cnn(CC(F)F)c3n2)C[C@@H]1COc1cccnc1C(F)(F)F] | 298b |
| 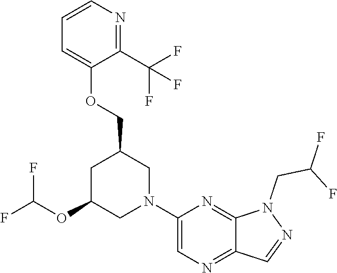 [SMILES: FC(F)Cn1ncc2ncc(N3C[C@H](COc4cccnc4C(F)(F)F)C[C@H](OC(F)F)C3)nc21] | 299a |
| 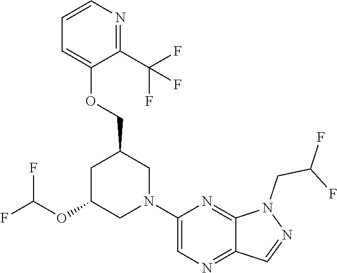 [SMILES: FC(F)Cn1ncc2ncc(N3C[C@H](COc4cccnc4C(F)(F)F)C[C@@H](OC(F)F)C3)nc21] | 299b |
| 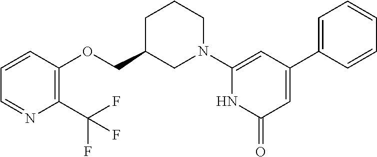 [SMILES: O=c1cc(-c2ccccc2)cc(N2CCC[C@H](COc3cccnc3C(F)(F)F)C2)[nH]1] | 300 |
| 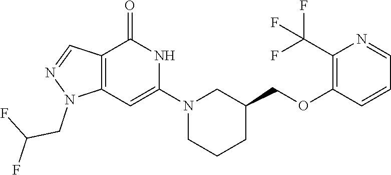 [SMILES: O=c1[nH]c(N2CCC[C@H](COc3cccnc3C(F)(F)F)C2)cc2c1cnn2CC(F)F] | 306 |
| 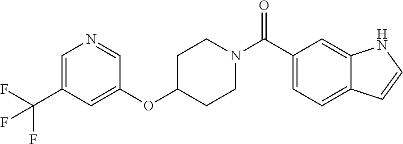 [SMILES: O=C(c1ccc2cc[nH]c2c1)N1CCC(Oc2cncc(C(F)(F)F)c2)CC1] | 307 |
| 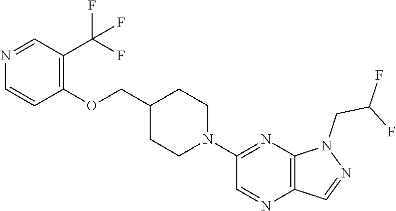 [SMILES: FC(F)Cn1ncc2ncc(N3CCC(COc4ccncc4C(F)(F)F)CC3)nc21] | 308 |
| 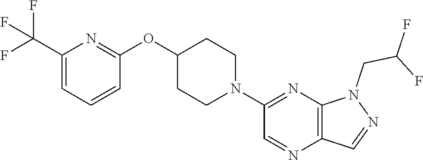 [SMILES: FC(F)Cn1ncc2ncc(N3CCC(Oc4cccc(C(F)(F)F)n4)CC3)nc21] | 309 |
| 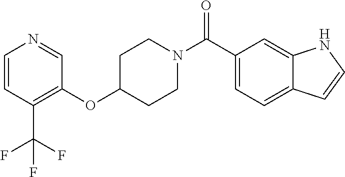 [SMILES: O=C(c1ccc2cc[nH]c2c1)N1CCC(Oc2cnccc2C(F)(F)F)CC1] | 310 |
| 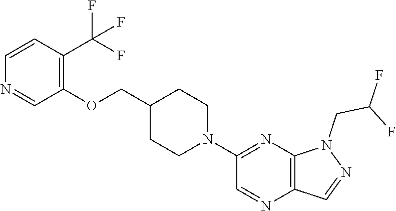 [SMILES: FC(F)Cn1ncc2ncc(N3CCC(COc4cnccc4C(F)(F)F)CC3)nc21] | 311 |
| 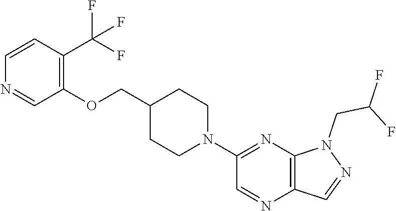 [SMILES: FC(F)Cn1ncc2ncc(N3CCC(COc4cnccc4C(F)(F)F)CC3)nc21] | 312 |
| 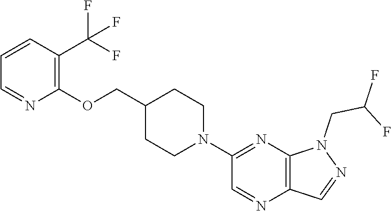 [SMILES: FC(F)Cn1ncc2ncc(N3CCC(COc4ncccc4C(F)(F)F)CC3)nc21] | 313 |
| 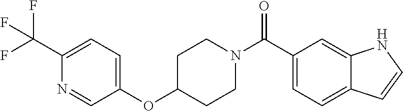 [SMILES: O=C(c1ccc2cc[nH]c2c1)N1CCC(Oc2ccc(C(F)(F)F)nc2)CC1] | 314 |
| 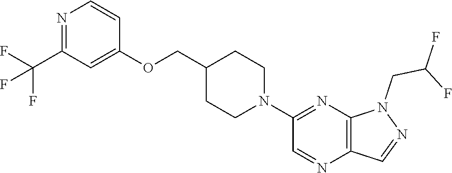 [SMILES: FC(F)Cn1ncc2ncc(N3CCC(COc4ccnc(C(F)(F)F)c4)CC3)nc21] | 315 |
| 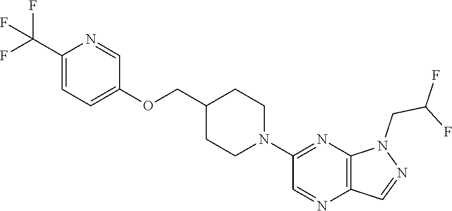 [SMILES: FC(F)Cn1ncc2ncc(N3CCC(COc4ccc(C(F)(F)F)nc4)CC3)nc21] | 316 |
| 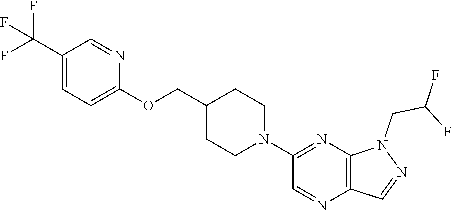 [SMILES: FC(F)Cn1ncc2ncc(N3CCC(COc4ccc(C(F)(F)F)cn4)CC3)nc21] | 317 |
| 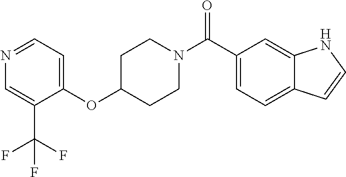 [SMILES: O=C(c1ccc2cc[nH]c2c1)N1CCC(Oc2ccncc2C(F)(F)F)CC1] | 318 |
| 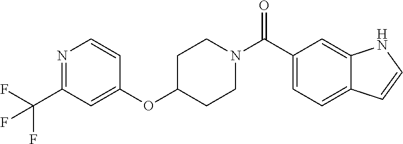 [SMILES: O=C(c1ccc2cc[nH]c2c1)N1CCC(Oc2ccnc(C(F)(F)F)c2)CC1] | 319 |
| 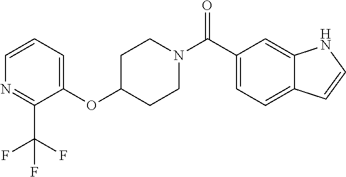 [SMILES: O=C(c1ccc2cc[nH]c2c1)N1CCC(Oc2cccnc2C(F)(F)F)CC1] | 320 |
| 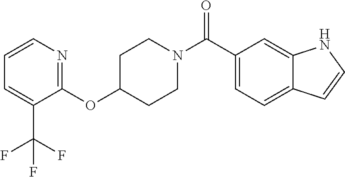 [SMILES: O=C(c1ccc2cc[nH]c2c1)N1CCC(Oc2ncccc2C(F)(F)F)CC1] | 321 |
| 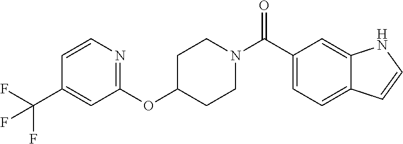 [SMILES: O=C(c1ccc2cc[nH]c2c1)N1CCC(Oc2cc(C(F)(F)F)ccn2)CC1] | 322 |
| 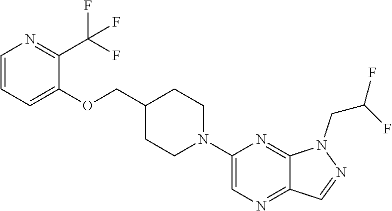 [SMILES: FC(F)Cn1ncc2ncc(N3CCC(COc4cccnc4C(F)(F)F)CC3)nc21] | 323 |
| 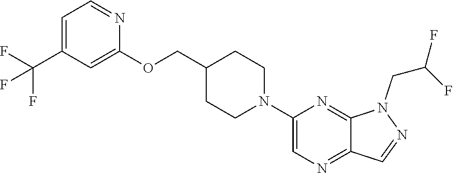 [SMILES: FC(F)Cn1ncc2ncc(N3CCC(COc4cc(C(F)(F)F)ccn4)CC3)nc21] | 324 |
| 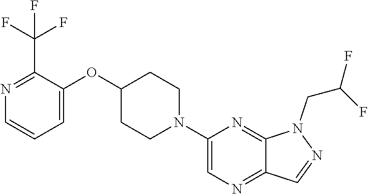 [SMILES: FC(F)Cn1ncc2ncc(N3CCC(Oc4cccnc4C(F)(F)F)CC3)nc21] | 325 |
| 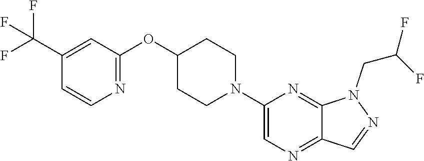 [SMILES: FC(F)Cn1ncc2ncc(N3CCC(Oc4cc(C(F)(F)F)ccn4)CC3)nc21] | 326 |
| 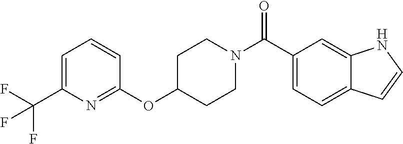 [SMILES: O=C(c1ccc2cc[nH]c2c1)N1CCC(Oc2cccc(C(F)(F)F)n2)CC1] | 327 |
| 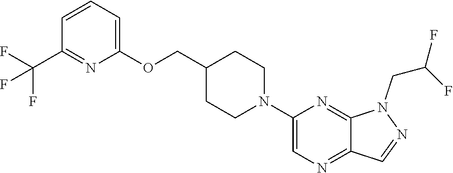 [SMILES: FC(F)Cn1ncc2ncc(N3CCC(COc4cccc(C(F)(F)F)n4)CC3)nc21] | 328 |
| 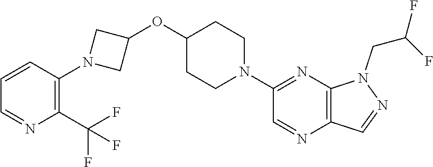 [SMILES: FC(F)Cn1ncc2ncc(N3CCC(OC4CN(c5cccnc5C(F)(F)F)C4)CC3)nc21] | 329 |
| 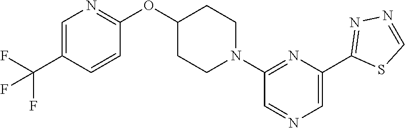 [SMILES: FC(F)(F)c1ccc(OC2CCN(c3cncc(-c4nncs4)n3)CC2)nc1] | 330 |
| 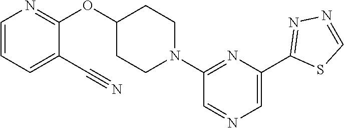 [SMILES: N#Cc1cccnc1OC1CCN(c2cncc(-c3nncs3)n2)CC1] | 331 |
| 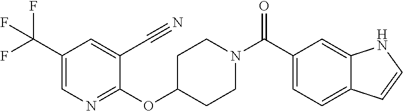 [SMILES: N#Cc1cc(C(F)(F)F)cnc1OC1CCN(C(=O)c2ccc3cc[nH]c3c2)CC1] | 332 |
| 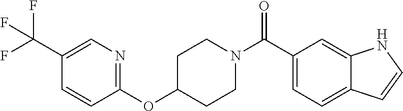 [SMILES: O=C(c1ccc2cc[nH]c2c1)N1CCC(Oc2ccc(C(F)(F)F)cn2)CC1] | 333 |
| 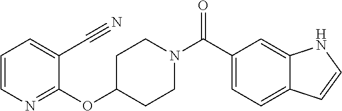 [SMILES: N#Cc1cccnc1OC1CCN(C(=O)c2ccc3cc[nH]c3c2)CC1] | 334 |
| 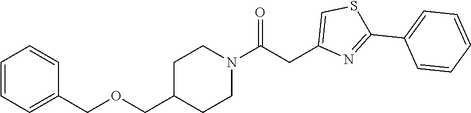 [SMILES: O=C(Cc1csc(-c2ccccc2)n1)N1CCC(COCc2ccccc2)CC1] | 335 |
| 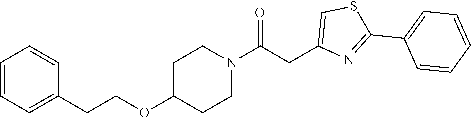 [SMILES: O=C(Cc1csc(-c2ccccc2)n1)N1CCC(OCCc2ccccc2)CC1] | 336 |
| 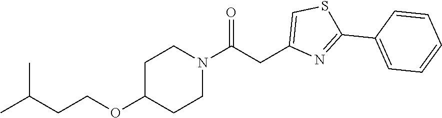 [SMILES: CC(C)CCOC1CCN(C(=O)Cc2csc(-c3ccccc3)n2)CC1] | 337 |
| 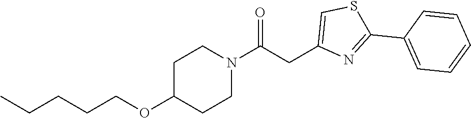 [SMILES: CCCCCOC1CCN(C(=O)Cc2csc(-c3ccccc3)n2)CC1] | 338 |
| 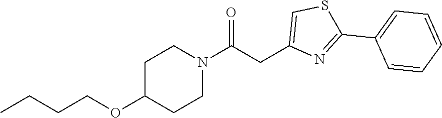 [SMILES: CCCCOC1CCN(C(=O)Cc2csc(-c3ccccc3)n2)CC1] | 339 |
| 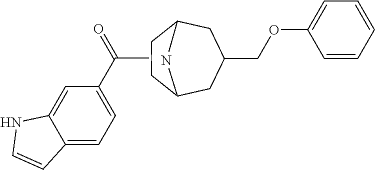 | 340 |
| 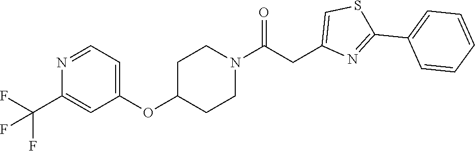 [SMILES: O=C(Cc1csc(-c2ccccc2)n1)N1CCC(Oc2ccnc(C(F)(F)F)c2)CC1] | 399 |
| 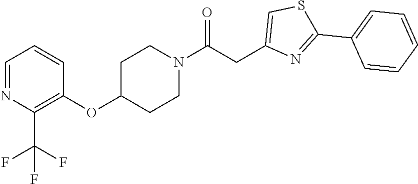 [SMILES: O=C(Cc1csc(-c2ccccc2)n1)N1CCC(Oc2cccnc2C(F)(F)F)CC1] | 400 |
| 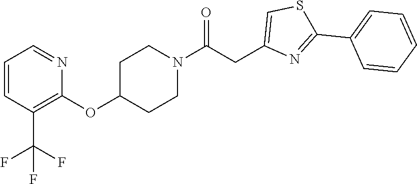 [SMILES: O=C(Cc1csc(-c2ccccc2)n1)N1CCC(Oc2ncccc2C(F)(F)F)CC1] | 401 |
| 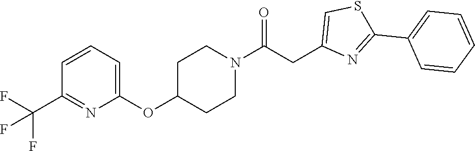 [SMILES: O=C(Cc1csc(-c2ccccc2)n1)N1CCC(Oc2cccc(C(F)(F)F)n2)CC1] | 402 |
| 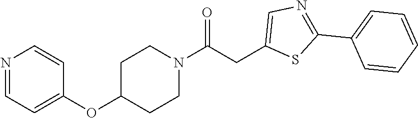 [SMILES: O=C(Cc1cnc(-c2ccccc2)s1)N1CCC(Oc2ccncc2)CC1] | 403 |
| 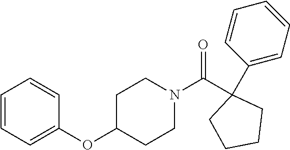 [SMILES: O=C(N1CCC(Oc2ccccc2)CC1)C1(c2ccccc2)CCCC1] | 404 |
| 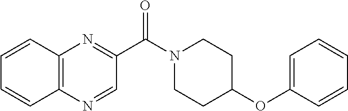 [SMILES: O=C(c1cnc2ccccc2n1)N1CCC(Oc2ccccc2)CC1] | 405 |
| 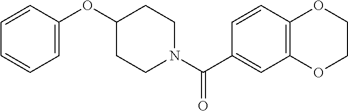 [SMILES: O=C(c1ccc2c(c1)OCCO2)N1CCC(Oc2ccccc2)CC1] | 406 |
| 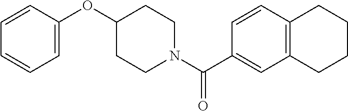 [SMILES: O=C(c1ccc2c(c1)CCCC2)N1CCC(Oc2ccccc2)CC1] | 407 |
| 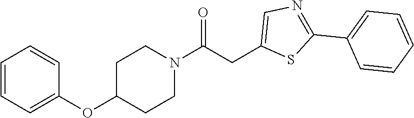 [SMILES: O=C(Cc1cnc(-c2ccccc2)s1)N1CCC(Oc2ccccc2)CC1] | 408 |
| 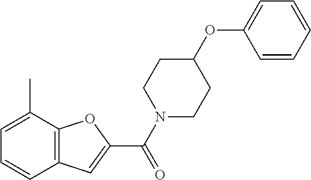 [SMILES: Cc1cccc2cc(C(=O)N3CCC(Oc4ccccc4)CC3)oc12] | 409 |
| 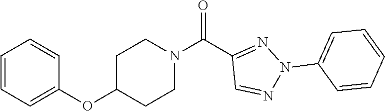 [SMILES: O=C(c1cnn(-c2ccccc2)n1)N1CCC(Oc2ccccc2)CC1] | 410 |
| 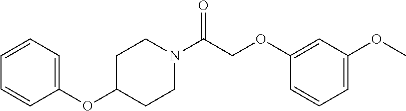 [SMILES: COc1cccc(OCC(=O)N2CCC(Oc3ccccc3)CC2)c1] | 411 |
| 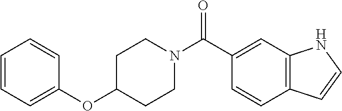 [SMILES: O=C(c1ccc2cc[nH]c2c1)N1CCC(Oc2ccccc2)CC1] | 412 |
| 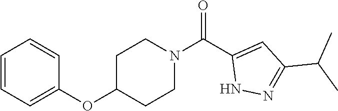 [SMILES: CC(C)c1cc(C(=O)N2CCC(Oc3ccccc3)CC2)[nH]n1] | 413 |
| 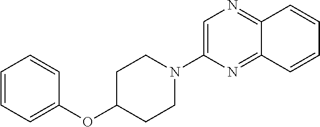 [SMILES: c1ccc(OC2CCN(c3cnc4ccccc4n3)CC2)cc1] | 414 |
| 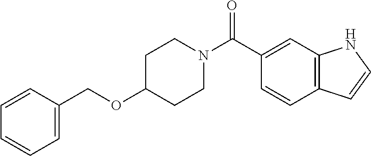 [SMILES: O=C(c1ccc2cc[nH]c2c1)N1CCC(OCc2ccccc2)CC1] | 415 |
| 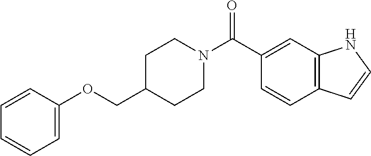 [SMILES: O=C(c1ccc2cc[nH]c2c1)N1CCC(COc2ccccc2)CC1] | 416 |
| 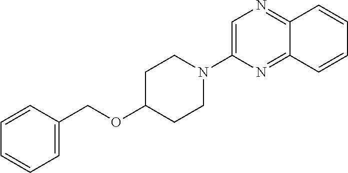 [SMILES: c1ccc(COC2CCN(c3cnc4ccccc4n3)CC2)cc1] | 417 |
| 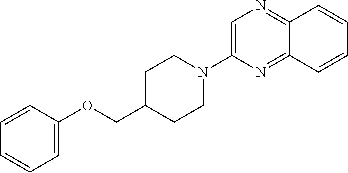 [SMILES: c1ccc(OCC2CCN(c3cnc4ccccc4n3)CC2)cc1] | 418 |

In certain embodiments, the compound of Formula (I) is a compound of Table 2, or a pharmaceutically acceptable salt, co-crystal, tautomer, stereoisomer, solvate, hydrate, polymorph, isotopically enriched derivative, or prodrug thereof. In certain embodiments, the compound of Formula (I) is not one or more of the compounds of Table 2, or a pharmaceutically acceptable salt, co-crystal, tautomer, stereoisomer, solvate, hydrate, polymorph, isotopically enriched derivative, or prodrug thereof.

TABLE 2

| | |
|---|---|
| 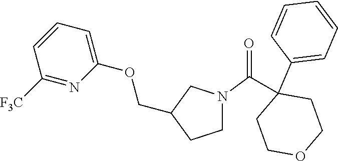 [SMILES: O=C(N1CCC(COc2cccc(C(F)(F)F)n2)C1)C1(c2ccccc2)CCOCC1] | 6 |
| 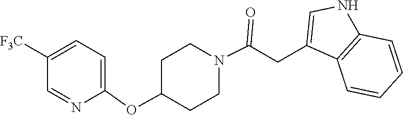 [SMILES: O=C(Cc1c[nH]c2ccccc12)N1CCC(Oc2ccc(C(F)(F)F)cn2)CC1] | 7 |
| 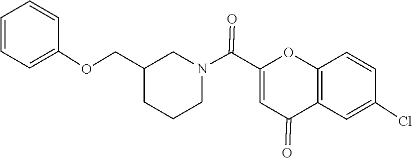 [SMILES: O=C(c1cc(=O)c2cc(Cl)ccc2o1)N1CCCC(COc2ccccc2)C1] | 8 |
| 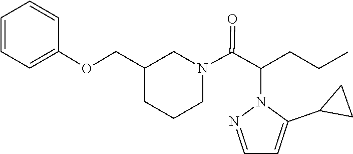 [SMILES: CCCC(C(=O)N1CCCC(COc2ccccc2)C1)n1nccc1C1CC1] | 9 |
| 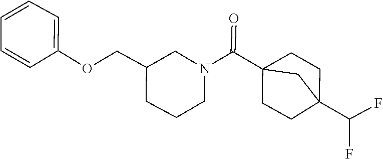 [SMILES: O=C(N1CCCC(COc2ccccc2)C1)C12CCC(C(F)F)(CC1)C2] | 10 |
| 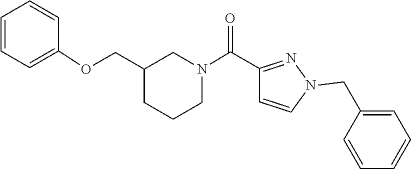 [SMILES: O=C(c1ccn(Cc2ccccc2)n1)N1CCCC(COc2ccccc2)C1] | 11 |
| 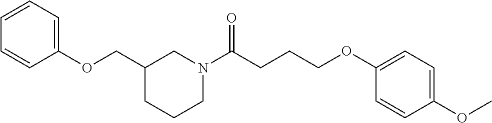 [SMILES: COc1ccc(OCCCC(=O)N2CCCC(COc3ccccc3)C2)cc1] | 12 |
| 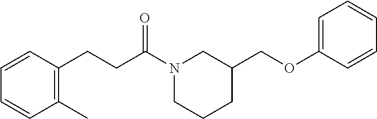 [SMILES: Cc1ccccc1CCC(=O)N1CCCC(COc2ccccc2)C1] | 129 |
| 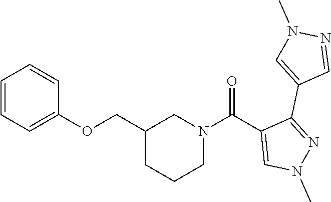 [SMILES: Cn1cc(-c2nn(C)cc2C(=O)N2CCCC(COc3ccccc3)C2)cn1] | 130 |
| 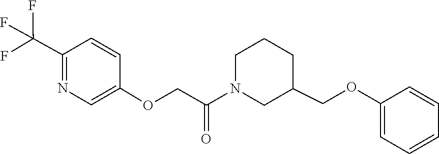 [SMILES: O=C(COc1ccc(C(F)(F)F)nc1)N1CCCC(COc2ccccc2)C1] | 131 |
| 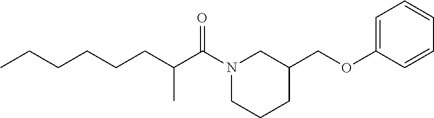 [SMILES: CCCCCCC(C)C(=O)N1CCCC(COc2ccccc2)C1] | 132 |
| 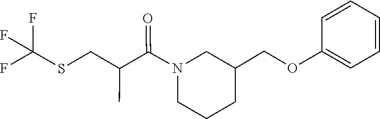 [SMILES: CC(CSC(F)(F)F)C(=O)N1CCCC(COc2ccccc2)C1] | 133 |
| 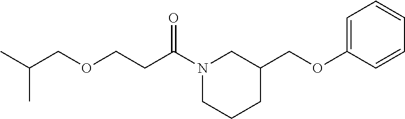 [SMILES: CC(C)COCCC(=O)N1CCCC(COc2ccccc2)C1] | 134 |
| 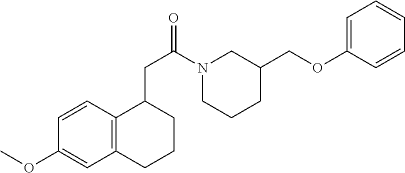 [SMILES: COc1ccc2c(c1)CCCC2CC(=O)N1CCCC(COc2ccccc2)C1] | 135 |
| 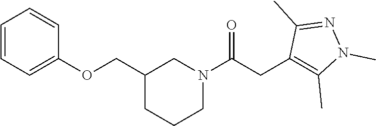 [SMILES: Cc1nn(C)c(C)c1CC(=O)N1CCCC(COc2ccccc2)C1] | 136 |
| 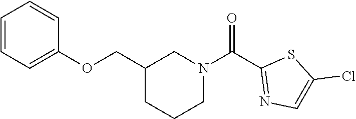 [SMILES: O=C(c1ncc(Cl)s1)N1CCCC(COc2ccccc2)C1] | 137 |
| 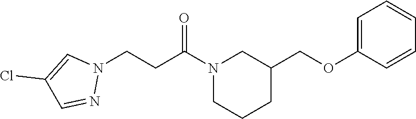 [SMILES: O=C(CCn1cc(Cl)cn1)N1CCCC(COc2ccccc2)C1] | 138 |
| 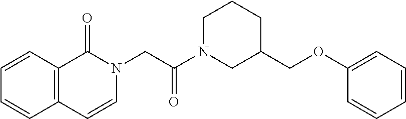 [SMILES: O=C(Cn1ccc2ccccc2c1=O)N1CCCC(COc2ccccc2)C1] | 139 |
| 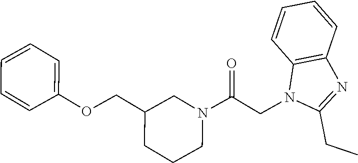 [SMILES: CCc1nc2ccccc2n1CC(=O)N1CCCC(COc2ccccc2)C1] | 140 |
| 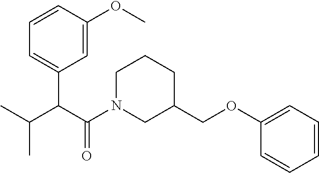 [SMILES: COc1cccc(C(C(=O)N2CCCC(COc3ccccc3)C2)C(C)C)c1] | 141 |
| 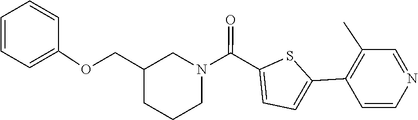 [SMILES: Cc1cnccc1-c1ccc(C(=O)N2CCCC(COc3ccccc3)C2)s1] | 142 |
| 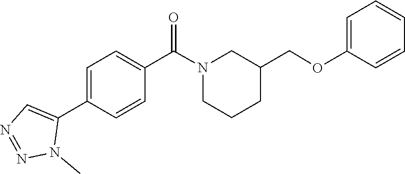 [SMILES: Cn1nncc1-c1ccc(C(=O)N2CCCC(COc3ccccc3)C2)cc1] | 143 |
| 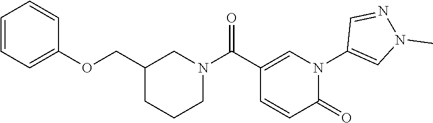 [SMILES: Cn1cc(-n2cc(C(=O)N3CCCC(COc4ccccc4)C3)ccc2=O)cn1] | 144 |
| 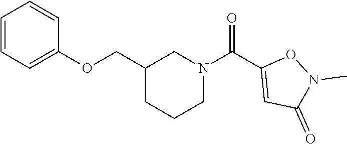 [SMILES: Cn1oc(C(=O)N2CCCC(COc3ccccc3)C2)cc1=O] | 145 |
| 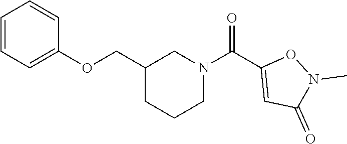 [SMILES: Cn1oc(C(=O)N2CCCC(COc3ccccc3)C2)cc1=O] | 146 |
| 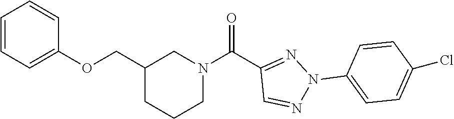 [SMILES: O=C(c1cnn(-c2ccc(Cl)cc2)n1)N1CCCC(COc2ccccc2)C1] | 147 |
| 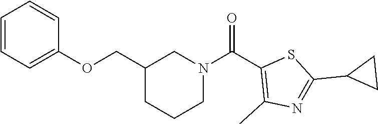 [SMILES: Cc1nc(C2CC2)sc1C(=O)N1CCCC(COc2ccccc2)C1] | 148 |
| 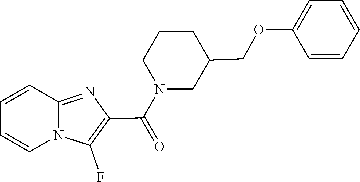 [SMILES: O=C(c1nc2ccccn2c1F)N1CCCC(COc2ccccc2)C1] | 149 |
| 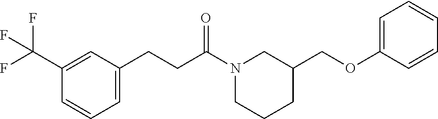 [SMILES: O=C(CCc1cccc(C(F)(F)F)c1)N1CCCC(COc2ccccc2)C1] | 150 |
| 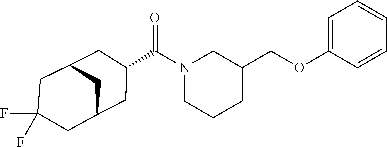 | 151 |
| 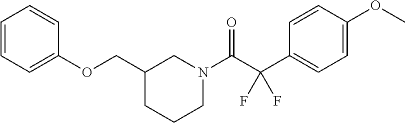 [SMILES: COc1ccc(C(F)(F)C(=O)N2CCCC(COc3ccccc3)C2)cc1] | 152 |
| 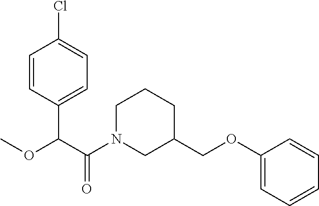 [SMILES: COC(C(=O)N1CCCC(COc2ccccc2)C1)c1ccc(Cl)cc1] | 153 |
| 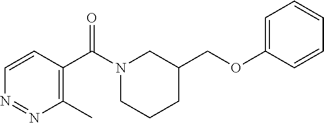 [SMILES: Cc1nnccc1C(=O)N1CCCC(COc2ccccc2)C1] | 154 |
| 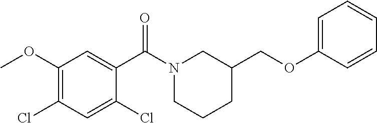 [SMILES: COc1cc(C(=O)N2CCCC(COc3ccccc3)C2)c(Cl)cc1Cl] | 155 |
| 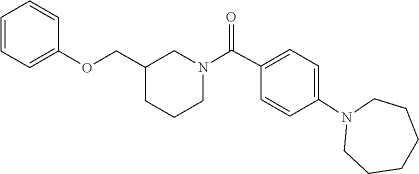 [SMILES: O=C(c1ccc(N2CCCCCC2)cc1)N1CCCC(COc2ccccc2)C1] | 156 |
| 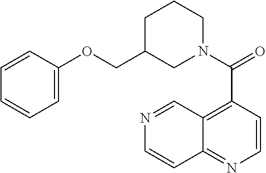 [SMILES: O=C(c1ccnc2ccncc12)N1CCCC(COc2ccccc2)C1] | 157 |
| 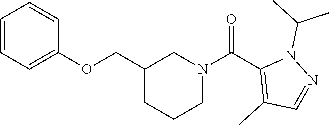 [SMILES: Cc1cnn(C(C)C)c1C(=O)N1CCCC(COc2ccccc2)C1] | 158 |
| 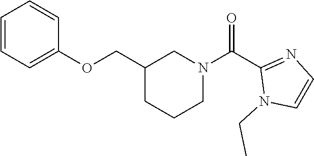 [SMILES: CCn1ccnc1C(=O)N1CCCC(COc2ccccc2)C1] | 159 |
| 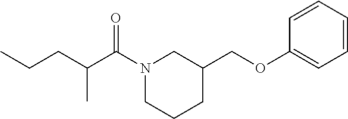 [SMILES: CCCC(C)C(=O)N1CCCC(COc2ccccc2)C1] | 160 |
| 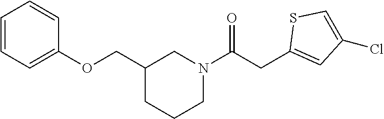 [SMILES: O=C(Cc1cc(Cl)cs1)N1CCCC(COc2ccccc2)C1] | 161 |
| 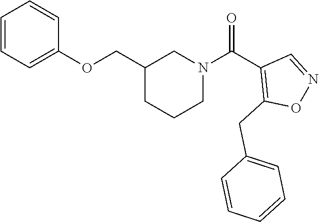 [SMILES: O=C(c1cnoc1Cc1ccccc1)N1CCCC(COc2ccccc2)C1] | 162 |
| 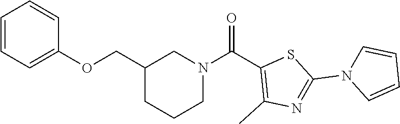 [SMILES: Cc1nc(-n2cccc2)sc1C(=O)N1CCCC(COc2ccccc2)C1] | 163 |
| 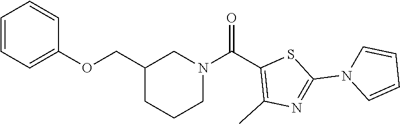 [SMILES: Cc1nc(-n2cccc2)sc1C(=O)N1CCCC(COc2ccccc2)C1] | 164 |
| 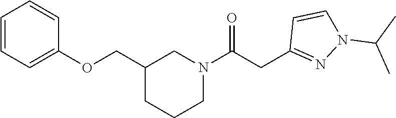 [SMILES: CC(C)n1ccc(CC(=O)N2CCCC(COc3ccccc3)C2)n1] | 165 |
| 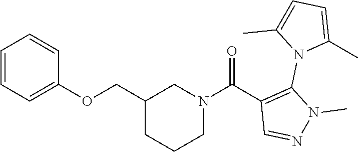 [SMILES: Cc1ccc(C)n1-c1c(C(=O)N2CCCC(COc3ccccc3)C2)cnn1C] | 166 |
| 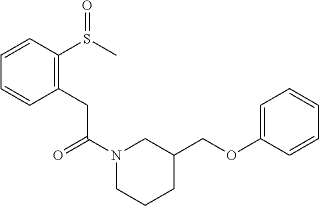 [SMILES: CS(=O)c1ccccc1CC(=O)N1CCCC(COc2ccccc2)C1] | 167 |
| 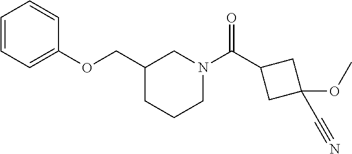 [SMILES: COC1(C#N)CC(C(=O)N2CCCC(COc3ccccc3)C2)C1] | 168 |
| 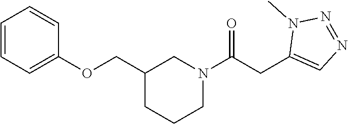 [SMILES: Cn1nncc1CC(=O)N1CCCC(COc2ccccc2)C1] | 169 |
| 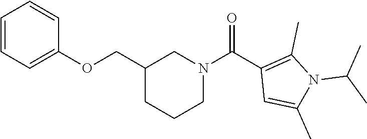 [SMILES: Cc1cc(C(=O)N2CCCC(COc3ccccc3)C2)c(C)n1C(C)C] | 170 |
| 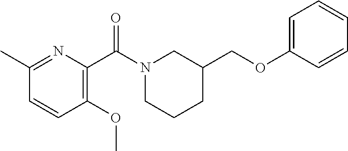 [SMILES: COc1ccc(C)nc1C(=O)N1CCCC(COc2ccccc2)C1] | 171 |
| 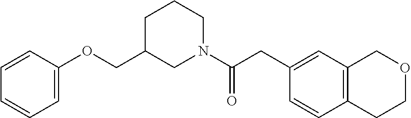 [SMILES: O=C(Cc1ccc2c(c1)COCC2)N1CCCC(COc2ccccc2)C1] | 172 |
| 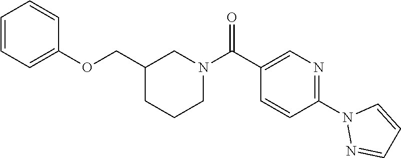 [SMILES: O=C(c1ccc(-n2cccn2)nc1)N1CCCC(COc2ccccc2)C1] | 173 |
| 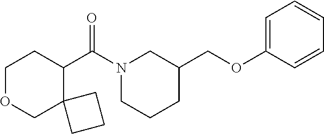 [SMILES: O=C(C1CCOCC12CCC2)N1CCCC(COc2ccccc2)C1] | 174 |
| 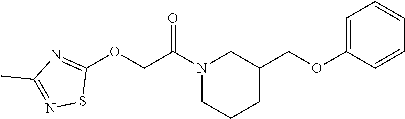 [SMILES: Cc1nsc(OCC(=O)N2CCCC(COc3ccccc3)C2)n1] | 175 |
| 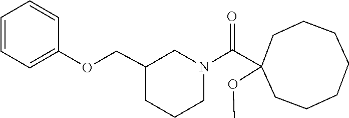 [SMILES: COC1(C(=O)N2CCCC(COc3ccccc3)C2)CCCCCCC1] | 176 |
| 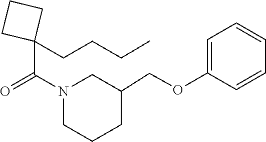 [SMILES: CCCCC1(C(=O)N2CCCC(COc3ccccc3)C2)CCC1] | 177 |
| 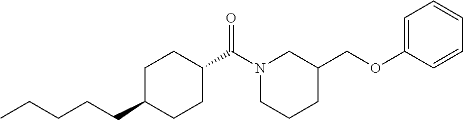 [SMILES: CCCCC[C@H]1CC[C@H](C(=O)N2CCCC(COc3ccccc3)C2)CC1] | 178 |
| 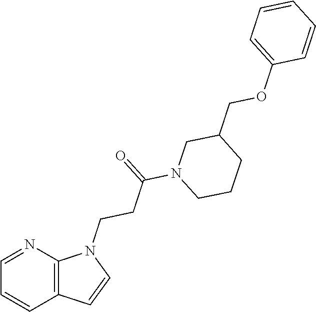 [SMILES: O=C(CCn1ccc2cccnc21)N1CCCC(COc2ccccc2)C1] | 179 |
| 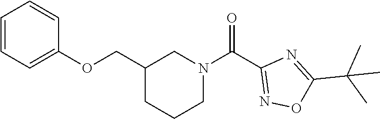 [SMILES: CC(C)(C)c1nc(C(=O)N2CCCC(COc3ccccc3)C2)no1] | 180 |
| 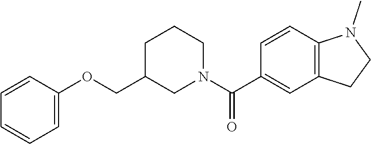 [SMILES: CN1CCc2cc(C(=O)N3CCCC(COc4ccccc4)C3)ccc21] | 181 |
| 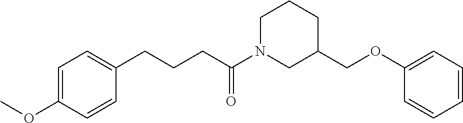 [SMILES: COc1ccc(CCCC(=O)N2CCCC(COc3ccccc3)C2)cc1] | 182 |
| 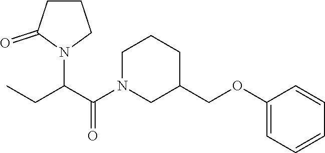 [SMILES: CCC(C(=O)N1CCCC(COc2ccccc2)C1)N1CCCC1=O] | 183 |
| 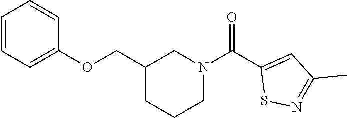 [SMILES: Cc1cc(C(=O)N2CCCC(COc3ccccc3)C2)sn1] | 184 |
| 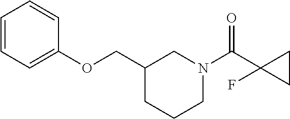 [SMILES: O=C(N1CCCC(COc2ccccc2)C1)C1(F)CC1] | 185 |
| 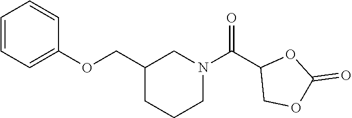 [SMILES: O=C1OCC(C(=O)N2CCCC(COc3ccccc3)C2)O1] | 186 |
| 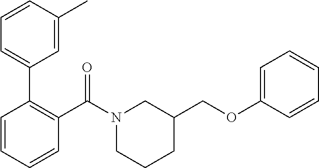 [SMILES: Cc1cccc(-c2ccccc2C(=O)N2CCCC(COc3ccccc3)C2)c1] | 187 |
| 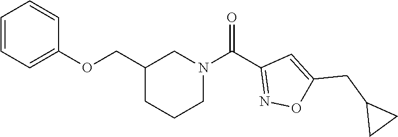 [SMILES: O=C(c1cc(CC2CC2)on1)N1CCCC(COc2ccccc2)C1] | 188 |
| 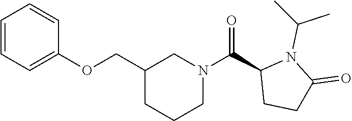 [SMILES: CC(C)N1C(=O)CC[C@H]1C(=O)N1CCCC(COc2ccccc2)C1] | 189 |
| 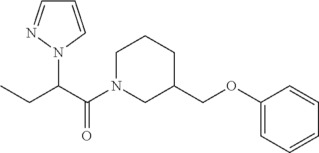 [SMILES: CCC(C(=O)N1CCCC(COc2ccccc2)C1)n1cccn1] | 190 |
| 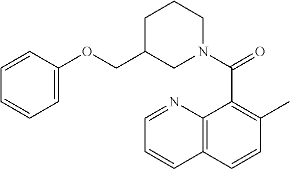 [SMILES: Cc1ccc2cccnc2c1C(=O)N1CCCC(COc2ccccc2)C1] | 191 |
| 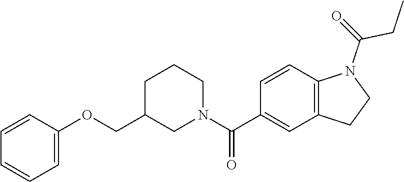 [SMILES: CCC(=O)N1CCc2cc(C(=O)N3CCCC(COc4ccccc4)C3)ccc21] | 192 |
| 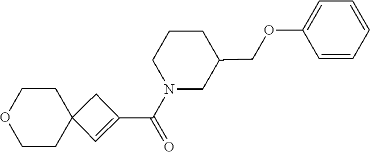 [SMILES: O=C(C1=CC2(CCOCC2)C1)N1CCCC(COc2ccccc2)C1] | 193 |
| 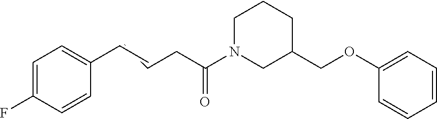 | 194 |
| 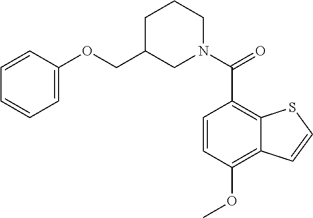 [SMILES: COc1ccc(C(=O)N2CCCC(COc3ccccc3)C2)c2sccc12] | 195 |
| 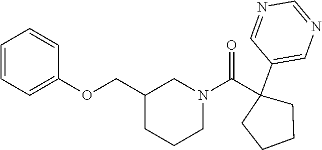 [SMILES: O=C(N1CCCC(COc2ccccc2)C1)C1(c2cncnc2)CCCC1] | 196 |
| 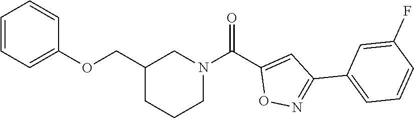 [SMILES: O=C(c1cc(-c2cccc(F)c2)no1)N1CCCC(COc2ccccc2)C1] | 197 |
| 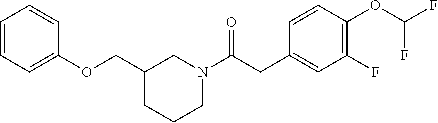 [SMILES: O=C(Cc1ccc(OC(F)F)c(F)c1)N1CCCC(COc2ccccc2)C1] | 198 |
| 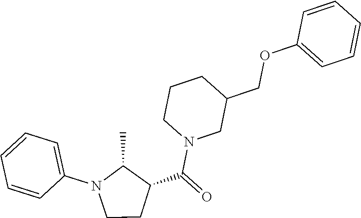 [SMILES: C[C@@H]1[C@H](C(=O)N2CCCC(COc3ccccc3)C2)CCN1c1ccccc1] | 199 |
| 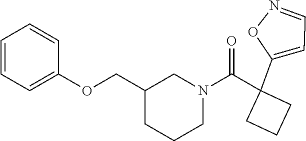 [SMILES: O=C(N1CCCC(COc2ccccc2)C1)C1(c2ccno2)CCC1] | 200 |
| 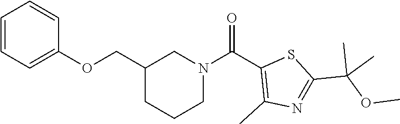 [SMILES: COC(C)(C)c1nc(C)c(C(=O)N2CCCC(COc3ccccc3)C2)s1] | 201 |
| 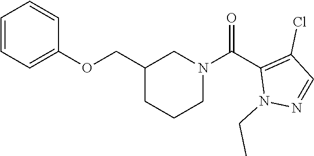 [SMILES: CCn1ncc(Cl)c1C(=O)N1CCCC(COc2ccccc2)C1] | 202 |
| 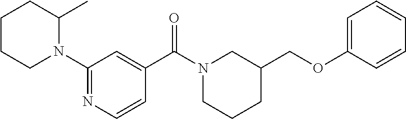 [SMILES: CC1CCCCN1c1cc(C(=O)N2CCCC(COc3ccccc3)C2)ccn1] | 203 |
| 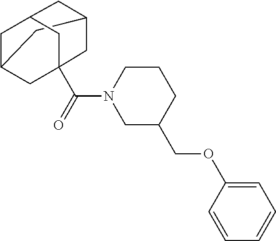 [SMILES: O=C(N1CCCC(COc2ccccc2)C1)C12CC3CC(CC(C3)C1)C2] | 204 |
| 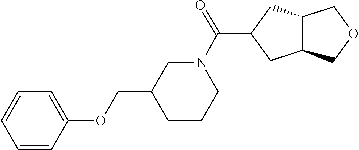 [SMILES: O=C(C1C[C@@H]2COC[C@H]2C1)N1CCCC(COc2ccccc2)C1] | 205 |
| 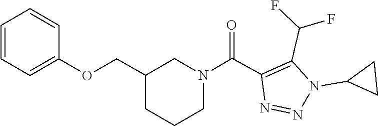 [SMILES: O=C(c1nnn(C2CC2)c1C(F)F)N1CCCC(COc2ccccc2)C1] | 206 |
| 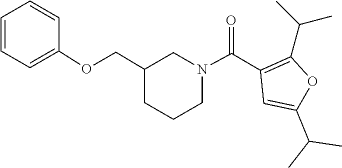 [SMILES: CC(C)c1cc(C(=O)N2CCCC(COc3ccccc3)C2)c(C(C)C)o1] | 207 |
| 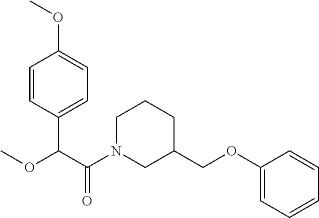 [SMILES: COc1ccc(C(OC)C(=O)N2CCCC(COc3ccccc3)C2)cc1] | 208 |
| 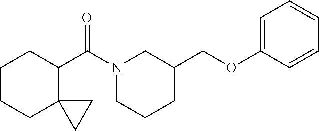 [SMILES: O=C(C1CCCCC12CC2)N1CCCC(COc2ccccc2)C1] | 209 |
| 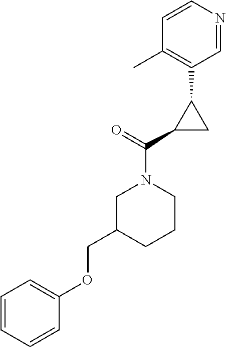 [SMILES: Cc1ccncc1[C@@H]1C[C@H]1C(=O)N1CCCC(COc2ccccc2)C1] | 210 |
| 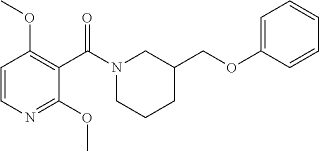 [SMILES: COc1ccnc(OC)c1C(=O)N1CCCC(COc2ccccc2)C1] | 211 |
| 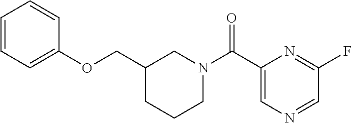 [SMILES: O=C(c1cncc(F)n1)N1CCCC(COc2ccccc2)C1] | 212 |
| 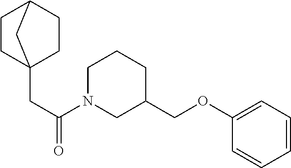 [SMILES: O=C(CC12CCC(CC1)C2)N1CCCC(COc2ccccc2)C1] | 213 |
| 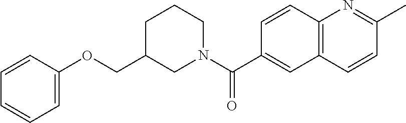 [SMILES: Cc1ccc2cc(C(=O)N3CCCC(COc4ccccc4)C3)ccc2n1] | 214 |
| 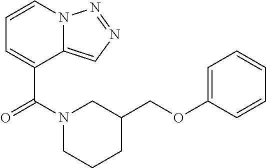 [SMILES: O=C(c1cccn2nncc12)N1CCCC(COc2ccccc2)C1] | 215 |
| 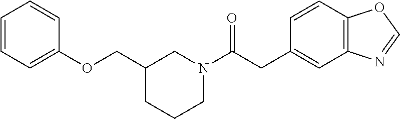 [SMILES: O=C(Cc1ccc2ocnc2c1)N1CCCC(COc2ccccc2)C1] | 216 |
| 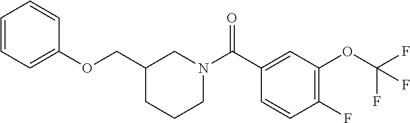 [SMILES: O=C(c1ccc(F)c(OC(F)(F)F)c1)N1CCCC(COc2ccccc2)C1] | 217 |
| 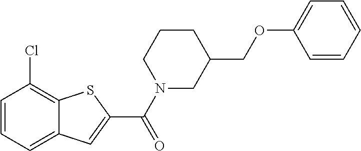 [SMILES: O=C(c1cc2cccc(Cl)c2s1)N1CCCC(COc2ccccc2)C1] | 218 |
| 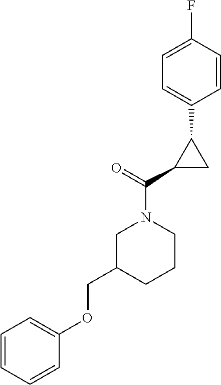 [SMILES: O=C([C@@H]1C[C@H]1c1ccc(F)cc1)N1CCCC(COc2ccccc2)C1] | 219 |
| 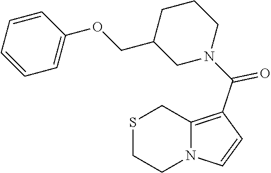 [SMILES: O=C(c1ccn2c1CSCC2)N1CCCC(COc2ccccc2)C1] | 220 |
| 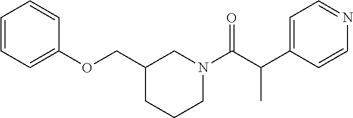 [SMILES: CC(C(=O)N1CCCC(COc2ccccc2)C1)c1ccncc1] | 221 |
| 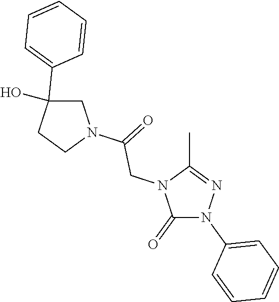 [SMILES: Cc1nn(-c2ccccc2)c(=O)n1CC(=O)N1CCC(O)(c2ccccc2)C1] | 301 |
| 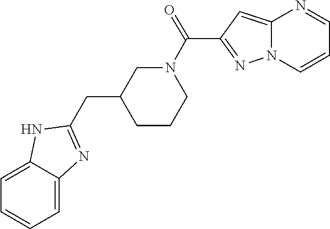 [SMILES: O=C(c1cc2ncccn2n1)N1CCCC(Cc2nc3ccccc3[nH]2)C1] | 302 |
| 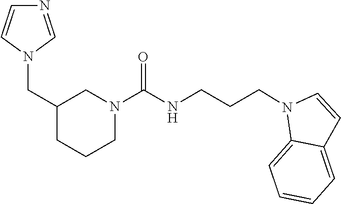 [SMILES: O=C(NCCCn1ccc2ccccc21)N1CCCC(Cn2ccnc2)C1] | 303 |
| 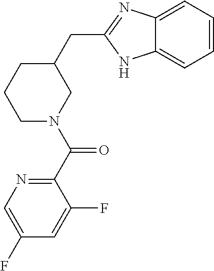 [SMILES: O=C(c1ncc(F)cc1F)N1CCCC(Cc2nc3ccccc3[nH]2)C1] | 304 |
| 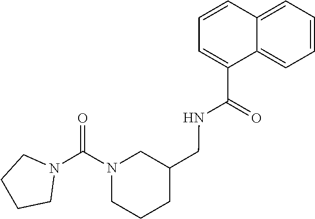 [SMILES: O=C(NCC1CCCN(C(=O)N2CCCC2)C1)c1cccc2ccccc12] | 305 |
| 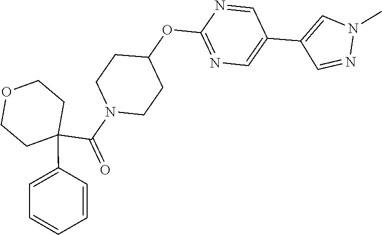 [SMILES: Cn1cc(-c2cnc(OC3CCN(C(=O)C4(c5ccccc5)CCOCC4)CC3)nc2)cn1] | 341 |
| 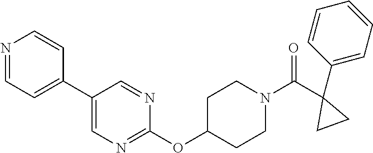 [SMILES: O=C(N1CCC(Oc2ncc(-c3ccncc3)cn2)CC1)C1(c2ccccc2)CC1] | 342 |
| 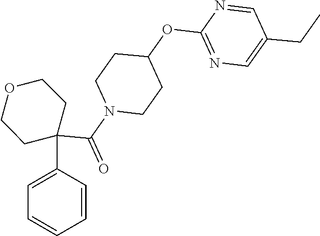 [SMILES: CCc1cnc(OC2CCN(C(=O)C3(c4ccccc4)CCOCC3)CC2)nc1] | 343 |
| 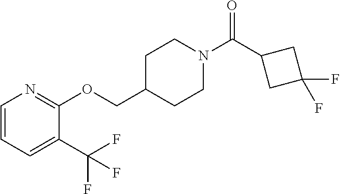 [SMILES: O=C(C1CC(F)(F)C1)N1CCC(COc2ncccc2C(F)(F)F)CC1] | 344 |
| 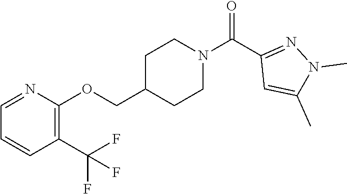 [SMILES: Cc1cc(C(=O)N2CCC(COc3ncccc3C(F)(F)F)CC2)nn1C] | 345 |
| 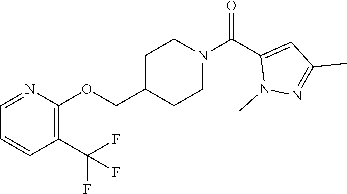 [SMILES: Cc1cc(C(=O)N2CCC(COc3ncccc3C(F)(F)F)CC2)n(C)n1] | 346 |
| 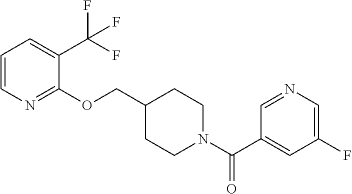 [SMILES: O=C(c1cncc(F)c1)N1CCC(COc2ncccc2C(F)(F)F)CC1] | 347 |
| 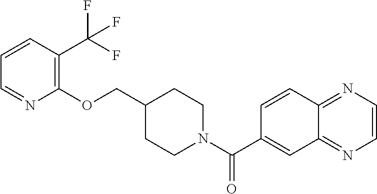 [SMILES: O=C(c1ccc2nccnc2c1)N1CCC(COc2ncccc2C(F)(F)F)CC1] | 348 |
| 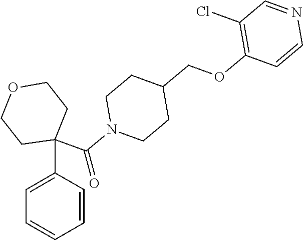 [SMILES: O=C(N1CCC(COc2ccncc2Cl)CC1)C1(c2ccccc2)CCOCC1] | 349 |
| 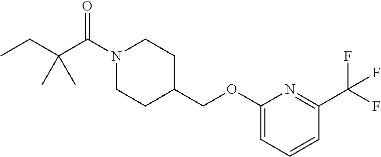 [SMILES: CCC(C)(C)C(=O)N1CCC(COc2cccc(C(F)(F)F)n2)CC1] | 350 |
| 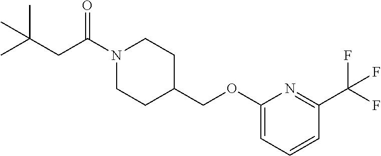 [SMILES: CC(C)(C)CC(=O)N1CCC(COc2cccc(C(F)(F)F)n2)CC1] | 351 |
| 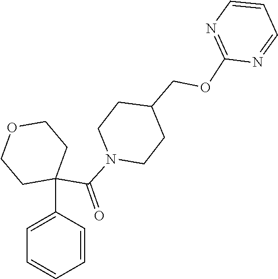 [SMILES: O=C(N1CCC(COc2ncccn2)CC1)C1(c2ccccc2)CCOCC1] | 352 |
| 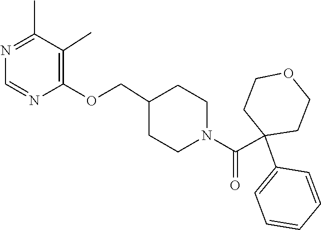 [SMILES: Cc1ncnc(OCC2CCN(C(=O)C3(c4ccccc4)CCOCC3)CC2)c1C] | 353 |
| 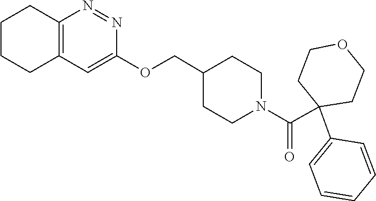 [SMILES: O=C(N1CCC(COc2cc3c(nn2)CCCC3)CC1)C1(c2ccccc2)CCOCC1] | 354 |
| 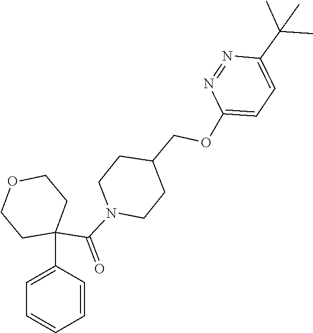 [SMILES: CC(C)(C)c1ccc(OCC2CCN(C(=O)C3(c4ccccc4)CCOCC3)CC2)nn1] | 355 |
| 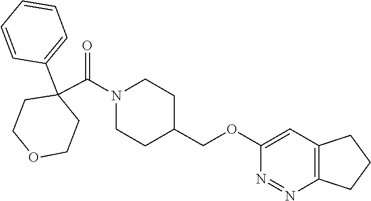 [SMILES: O=C(N1CCC(COc2cc3c(nn2)CCC3)CC1)C1(c2ccccc2)CCOCC1] | 356 |
| 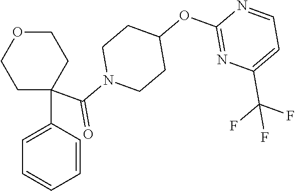 [SMILES: O=C(N1CCC(Oc2nccc(C(F)(F)F)n2)CC1)C1(c2ccccc2)CCOCC1] | 357 |
| 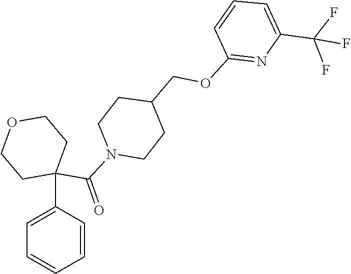 [SMILES: O=C(N1CCC(COc2cccc(C(F)(F)F)n2)CC1)C1(c2ccccc2)CCOCC1] | 358 |
| 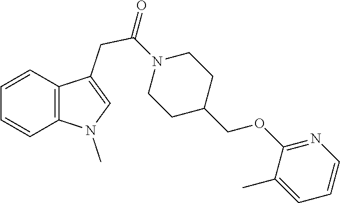 [SMILES: Cc1cccnc1OCC1CCN(C(=O)Cc2cn(C)c3ccccc23)CC1] | 359 |
| 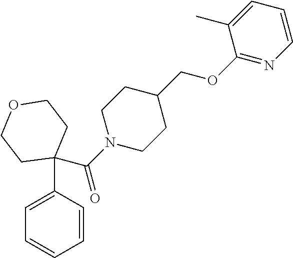 [SMILES: Cc1cccnc1OCC1CCN(C(=O)C2(c3ccccc3)CCOCC2)CC1] | 360 |
| 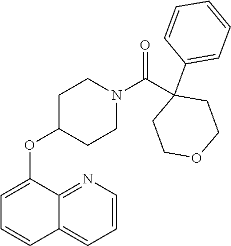 [SMILES: O=C(N1CCC(Oc2cccc3cccnc23)CC1)C1(c2ccccc2)CCOCC1] | 361 |
| 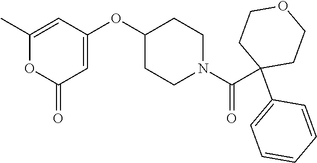 [SMILES: Cc1cc(OC2CCN(C(=O)C3(c4ccccc4)CCOCC3)CC2)cc(=O)o1] | 362 |
| 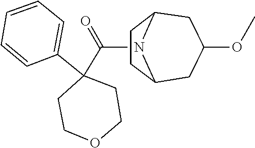 [SMILES: COC1CC2CCCC(C1)N2C(=O)C1(c2ccccc2)CCOCC1] | 363 |
| 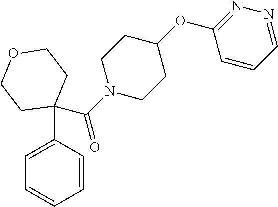 [SMILES: O=C(N1CCC(Oc2cccnn2)CC1)C1(c2ccccc2)CCOCC1] | 364 |
| 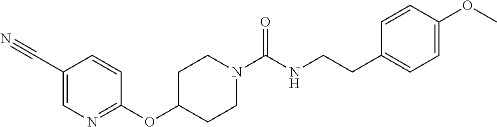 [SMILES: COc1ccc(CCNC(=O)N2CCC(Oc3ccc(C#N)cn3)CC2)cc1] | 365 |
| 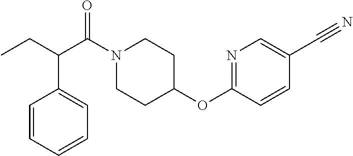 [SMILES: CCC(C(=O)N1CCC(Oc2ccc(C#N)cn2)CC1)c1ccccc1] | 366 |
| 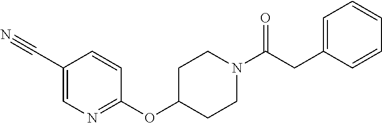 [SMILES: N#Cc1ccc(OC2CCN(C(=O)Cc3ccccc3)CC2)nc1] | 367 |
| 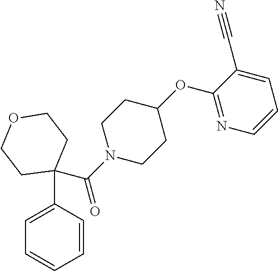 [SMILES: N#Cc1cccnc1OC1CCN(C(=O)C2(c3ccccc3)CCOCC2)CC1] | 368 |
| 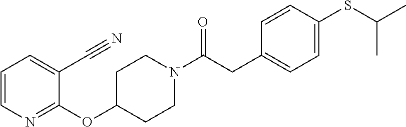 [SMILES: CC(C)Sc1ccc(CC(=O)N2CCC(Oc3ncccc3C#N)CC2)cc1] | 369 |
| 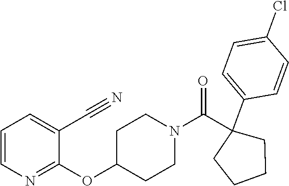 [SMILES: N#Cc1cccnc1OC1CCN(C(=O)C2(c3ccc(Cl)cc3)CCCC2)CC1] | 370 |
| 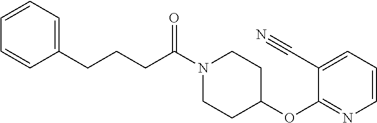 [SMILES: N#Cc1cccnc1OC1CCN(C(=O)CCCc2ccccc2)CC1] | 371 |
| 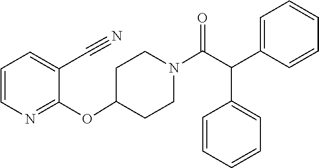 [SMILES: N#Cc1cccnc1OC1CCN(C(=O)C(c2ccccc2)c2ccccc2)CC1] | 372 |
| 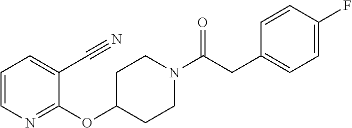 [SMILES: N#Cc1cccnc1OC1CCN(C(=O)Cc2ccc(F)cc2)CC1] | 373 |
| 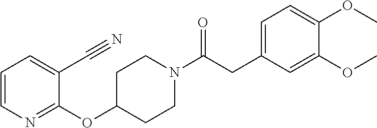 [SMILES: COc1ccc(CC(=O)N2CCC(Oc3ncccc3C#N)CC2)cc1OC] | 374 |
| 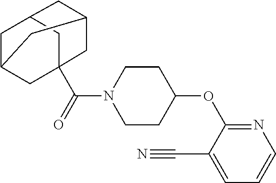 [SMILES: N#Cc1cccnc1OC1CCN(C(=O)C23CC4CC(CC(C4)C2)C3)CC1] | 375 |
| 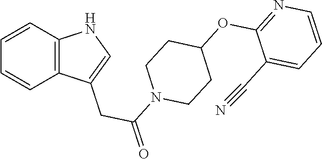 [SMILES: N#Cc1cccnc1OC1CCN(C(=O)Cc2c[nH]c3ccccc23)CC1] | 376 |
| 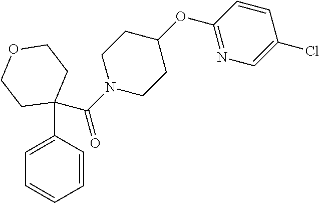 [SMILES: O=C(N1CCC(Oc2ccc(Cl)cn2)CC1)C1(c2ccccc2)CCOCC1] | 377 |
| 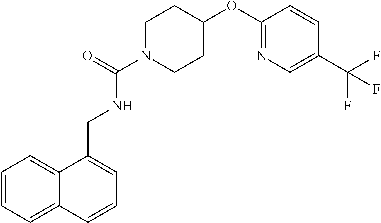 [SMILES: O=C(NCc1cccc2ccccc12)N1CCC(Oc2ccc(C(F)(F)F)cn2)CC1] | 378 |
| 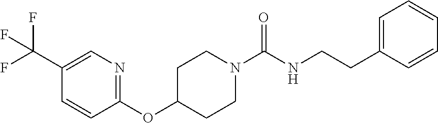 [SMILES: O=C(NCCc1ccccc1)N1CCC(Oc2ccc(C(F)(F)F)cn2)CC1] | 379 |
| 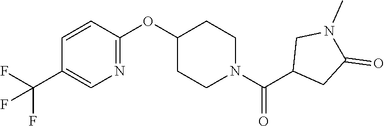 [SMILES: CN1CC(C(=O)N2CCC(Oc3ccc(C(F)(F)F)cn3)CC2)CC1=O] | 380 |
| 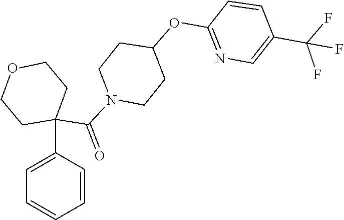 [SMILES: O=C(N1CCC(Oc2ccc(C(F)(F)F)cn2)CC1)C1(c2ccccc2)CCOCC1] | 381 |
| 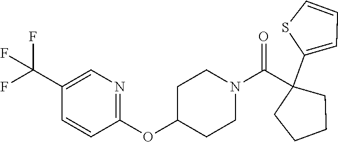 [SMILES: O=C(N1CCC(Oc2ccc(C(F)(F)F)cn2)CC1)C1(c2cccs2)CCCC1] | 382 |
| 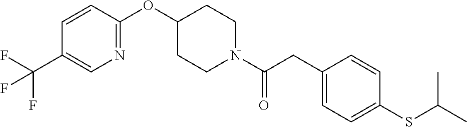 [SMILES: CC(C)Sc1ccc(CC(=O)N2CCC(Oc3ccc(C(F)(F)F)cn3)CC2)cc1] | 383 |
| 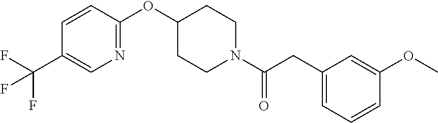 [SMILES: COc1cccc(CC(=O)N2CCC(Oc3ccc(C(F)(F)F)cn3)CC2)c1] | 384 |
| 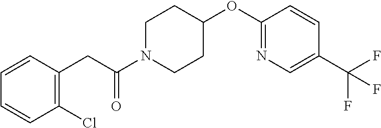 [SMILES: O=C(Cc1ccccc1Cl)N1CCC(Oc2ccc(C(F)(F)F)cn2)CC1] | 385 |
| 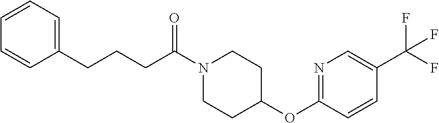 [SMILES: O=C(CCCc1ccccc1)N1CCC(Oc2ccc(C(F)(F)F)cn2)CC1] | 386 |
| 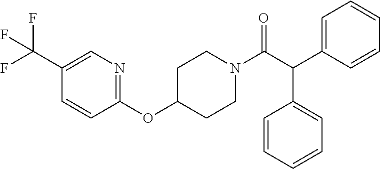 [SMILES: O=C(C(c1ccccc1)c1ccccc1)N1CCC(Oc2ccc(C(F)(F)F)cn2)CC1] | 387 |
| 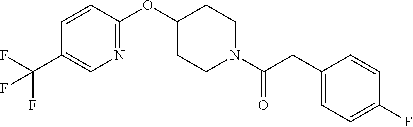 [SMILES: O=C(Cc1ccc(F)cc1)N1CCC(Oc2ccc(C(F)(F)F)cn2)CC1] | 388 |
| 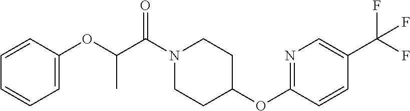 [SMILES: CC(Oc1ccccc1)C(=O)N1CCC(Oc2ccc(C(F)(F)F)cn2)CC1] | 389 |
| 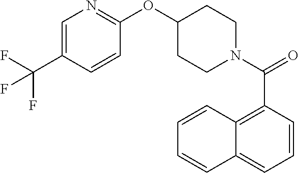 [SMILES: O=C(c1cccc2ccccc12)N1CCC(Oc2ccc(C(F)(F)F)cn2)CC1] | 390 |
| 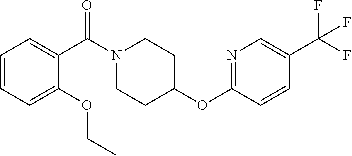 [SMILES: CCOc1ccccc1C(=O)N1CCC(Oc2ccc(C(F)(F)F)cn2)CC1] | 391 |
| 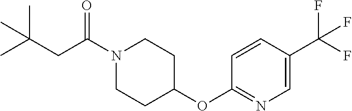 [SMILES: CC(C)(C)CC(=O)N1CCC(Oc2ccc(C(F)(F)F)cn2)CC1] | 392 |
| 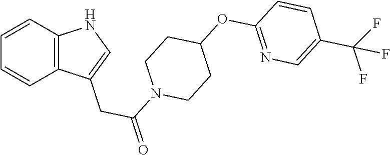 [SMILES: O=C(Cc1c[nH]c2ccccc12)N1CCC(Oc2ccc(C(F)(F)F)cn2)CC1] | 393 |
| 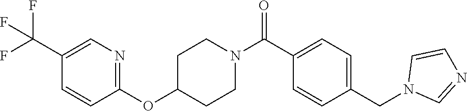 [SMILES: O=C(c1ccc(Cn2ccnc2)cc1)N1CCC(Oc2ccc(C(F)(F)F)cn2)CC1] | 394 |
| 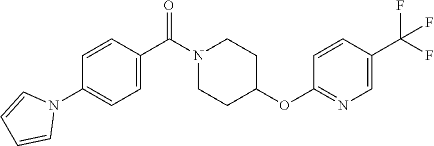 [SMILES: O=C(c1ccc(-n2cccc2)cc1)N1CCC(Oc2ccc(C(F)(F)F)cn2)CC1] | 395 |
| 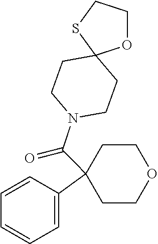 [SMILES: O=C(N1CCC2(CC1)OCCS2)C1(c2ccccc2)CCOCC1] | 396 |
| 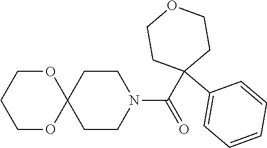 [SMILES: O=C(N1CCC2(CC1)OCCCO2)C1(c2ccccc2)CCOCC1] | 397 |
| 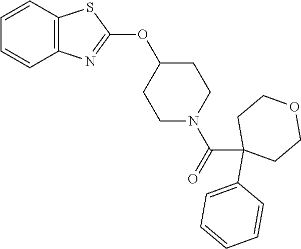 [SMILES: O=C(N1CCC(Oc2nc3ccccc3s2)CC1)C1(c2ccccc2)CCOCC1] | 398 |
| 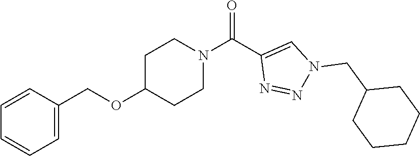 [SMILES: O=C(c1cn(CC2CCCCC2)nn1)N1CCC(OCc2ccccc2)CC1] | 419 |
| 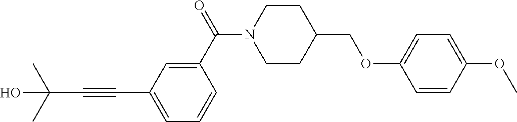 [SMILES: COc1ccc(OCC2CCN(C(=O)c3cccc(C#CC(C)(C)O)c3)CC2)cc1] | 420 |
| 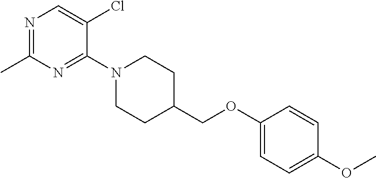 | 421 |
| 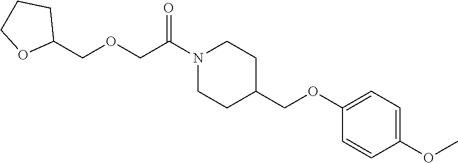 [SMILES: COc1ccc(OCC2CCN(C(=O)COCC3CCCO3)CC2)cc1] | 422 |
| 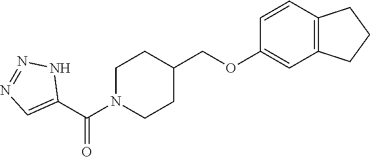 [SMILES: O=C(c1cnn[nH]1)N1CCC(COc2ccc3c(c2)CCC3)CC1] | 423 |
| 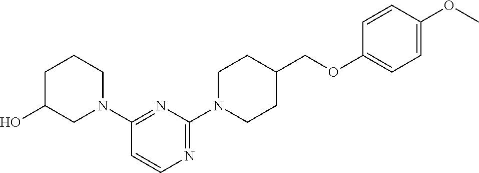 [SMILES: COc1ccc(OCC2CCN(c3nccc(N4CCCC(O)C4)n3)CC2)cc1] | 424 |
| 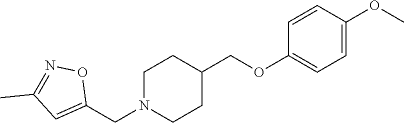 | 425 |
| 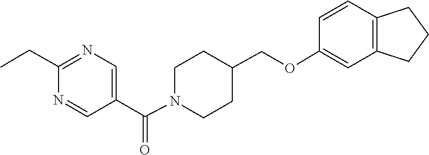 [SMILES: CCc1ncc(C(=O)N2CCC(COc3ccc4c(c3)CCC4)CC2)cn1] | 426 |
| 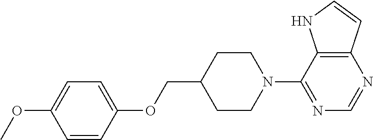 [SMILES: COc1ccc(OCC2CCN(c3ncnc4cc[nH]c34)CC2)cc1] | 427 |
| 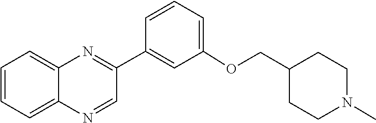 [SMILES: CN1CCC(COc2cccc(-c3cnc4ccccc4n3)c2)CC1] | 428 |
| 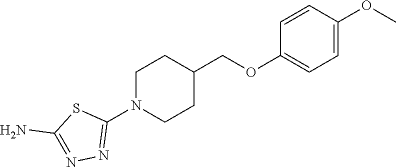 [SMILES: COc1ccc(OCC2CCN(c3nnc(N)s3)CC2)cc1] | 429 |
| 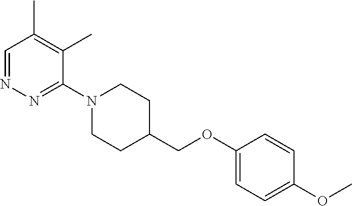 [SMILES: COc1ccc(OCC2CCN(c3nncc(C)c3C)CC2)cc1] | 430 |
| 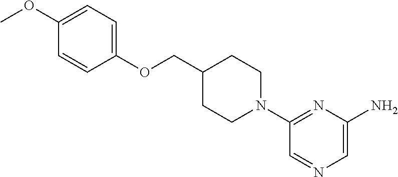 [SMILES: COc1ccc(OCC2CCN(c3cncc(N)n3)CC2)cc1] | 431 |
| 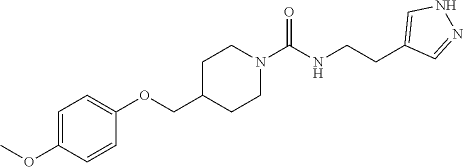 [SMILES: COc1ccc(OCC2CCN(C(=O)NCCc3cn[nH]c3)CC2)cc1] | 432 |
| 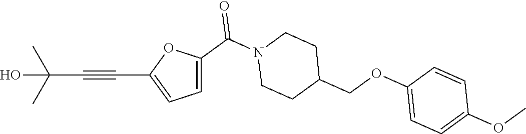 [SMILES: COc1ccc(OCC2CCN(C(=O)c3ccc(C#CC(C)(C)O)o3)CC2)cc1] | 433 |
| 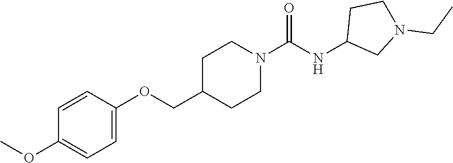 [SMILES: CCN1CCC(NC(=O)N2CCC(COc3ccc(OC)cc3)CC2)C1] | 434 |
| 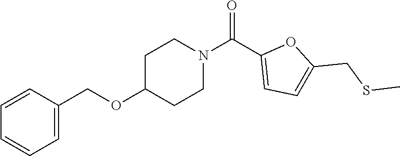 [SMILES: CSCc1ccc(C(=O)N2CCC(OCc3ccccc3)CC2)o1] | 435 |
| 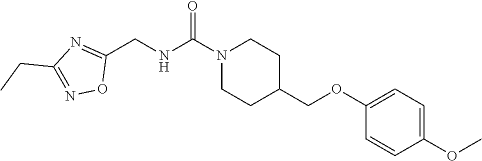 [SMILES: CCc1noc(CNC(=O)N2CCC(COc3ccc(OC)cc3)CC2)n1] | 436 |
| 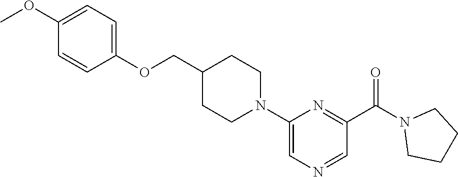 [SMILES: COc1ccc(OCC2CCN(c3cncc(C(=O)N4CCCC4)n3)CC2)cc1] | 437 |
| 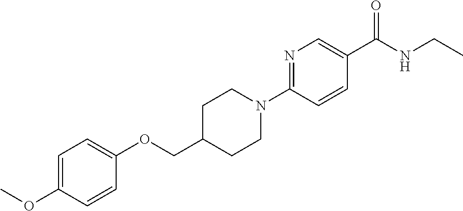 [SMILES: CCNC(=O)c1ccc(N2CCC(COc3ccc(OC)cc3)CC2)nc1] | 438 |
| 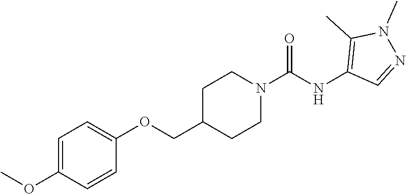 | 439 |
| 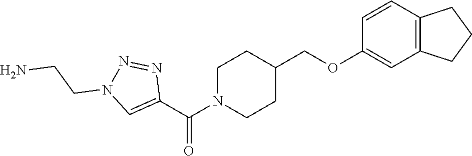 [SMILES: NCCn1cc(C(=O)N2CCC(COc3ccc4c(c3)CCC4)CC2)nn1] | 440 |
| 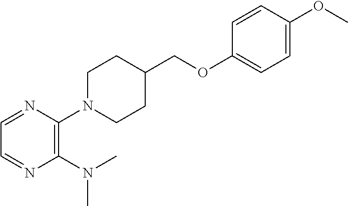 [SMILES: COc1ccc(OCC2CCN(c3nccnc3N(C)C)CC2)cc1] | 441 |
| 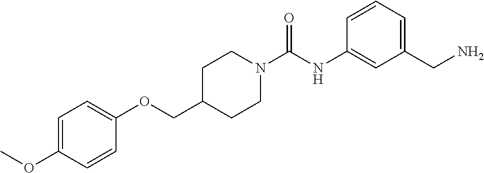 [SMILES: COc1ccc(OCC2CCN(C(=O)Nc3cccc(CN)c3)CC2)cc1] | 442 |
| 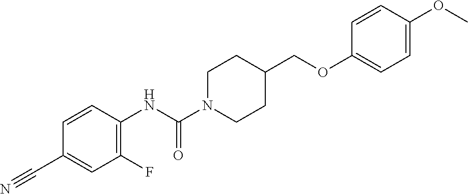 [SMILES: COc1ccc(OCC2CCN(C(=O)Nc3ccc(C#N)cc3F)CC2)cc1] | 443 |
| 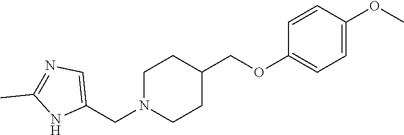 | 444 |
| 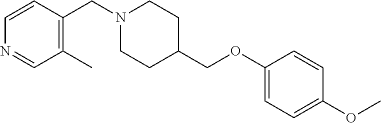 [SMILES: COc1ccc(OCC2CCN(Cc3ccncc3C)CC2)cc1] | 445 |
| 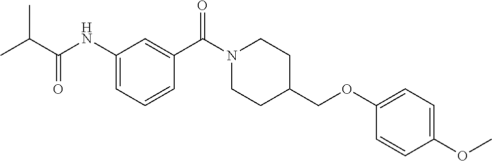 [SMILES: COc1ccc(OCC2CCN(C(=O)c3cccc(NC(=O)C(C)C)c3)CC2)cc1] | 446 |
| 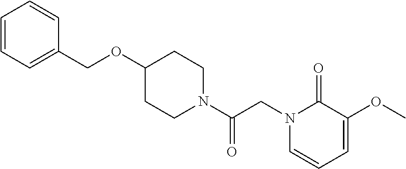 [SMILES: COc1cccn(CC(=O)N2CCC(OCc3ccccc3)CC2)c1=O] | 447 |
| 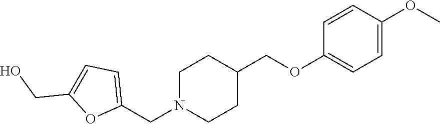 [SMILES: COc1ccc(OCC2CCN(Cc3ccc(CO)o3)CC2)cc1] | 448 |
| 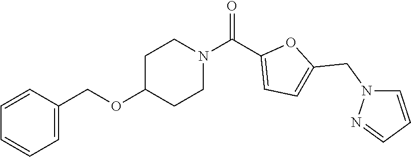 [SMILES: O=C(c1ccc(Cn2cccn2)o1)N1CCC(OCc2ccccc2)CC1] | 449 |
| 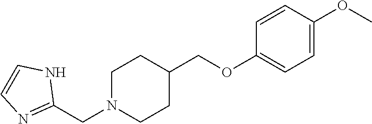 [SMILES: COc1ccc(OCC2CCN(Cc3ncc[nH]3)CC2)cc1] | 450 |
| 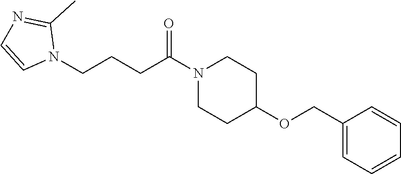 [SMILES: Cc1nccn1CCCC(=O)N1CCC(OCc2ccccc2)CC1] | 451 |
| 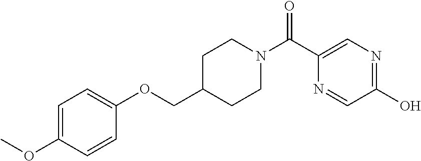 [SMILES: COc1ccc(OCC2CCN(C(=O)c3cnc(O)cn3)CC2)cc1] | 452 |
| 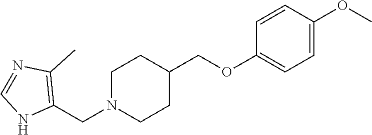 [SMILES: COc1ccc(OCC2CCN(Cc3[nH]cnc3C)CC2)cc1] | 453 |
| 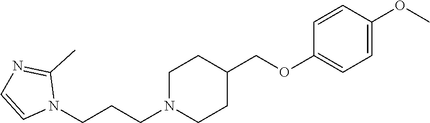 [SMILES: COc1ccc(OCC2CCN(CCCn3ccnc3C)CC2)cc1] | 454 |
| 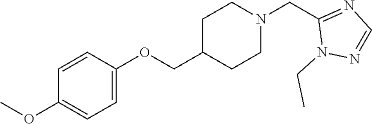 [SMILES: CCn1ncnc1CN1CCC(COc2ccc(OC)cc2)CC1] | 455 |
| 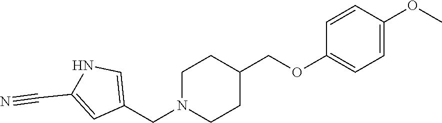 [SMILES: COc1ccc(OCC2CCN(Cc3c[nH]c(C#N)c3)CC2)cc1] | 456 |
| 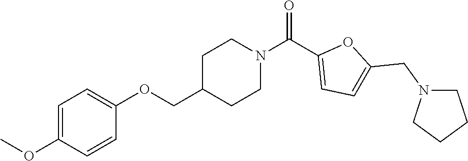 [SMILES: COc1ccc(OCC2CCN(C(=O)c3ccc(CN4CCCC4)o3)CC2)cc1] | 457 |
| 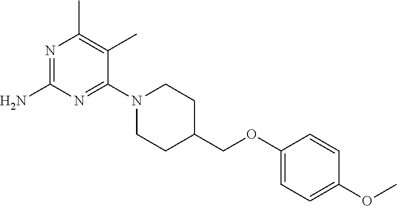 [SMILES: COc1ccc(OCC2CCN(c3nc(N)nc(C)c3C)CC2)cc1] | 458 |
| 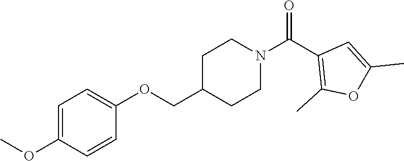 | 459 |
| 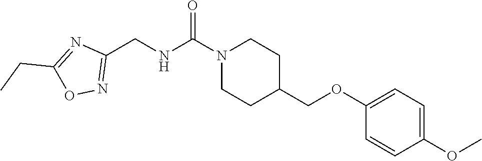 [SMILES: CCc1nc(CNC(=O)N2CCC(COc3ccc(OC)cc3)CC2)no1] | 460 |
| 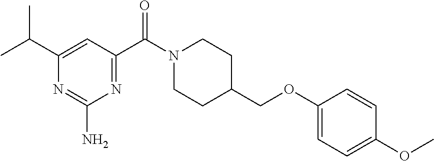 [SMILES: COc1ccc(OCC2CCN(C(=O)c3cc(C(C)C)nc(N)n3)CC2)cc1] | 461 |
| 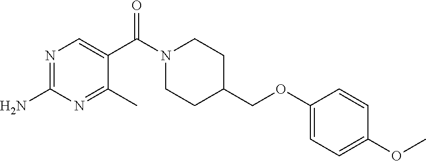 [SMILES: COc1ccc(OCC2CCN(C(=O)c3cnc(N)nc3C)CC2)cc1] | 462 |
| 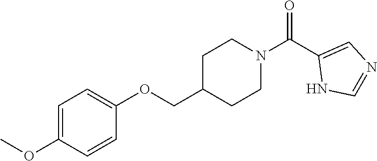 [SMILES: COc1ccc(OCC2CCN(C(=O)c3cnc[nH]3)CC2)cc1] | 463 |
| 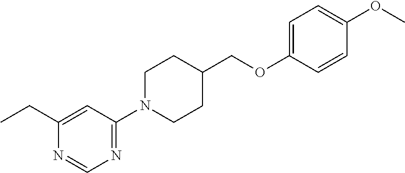 [SMILES: CCc1cc(N2CCC(COc3ccc(OC)cc3)CC2)ncn1] | 464 |
| 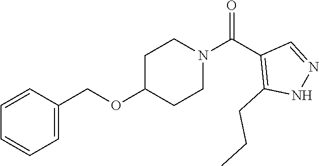 [SMILES: CCCc1[nH]ncc1C(=O)N1CCC(OCc2ccccc2)CC1] | 465 |
| 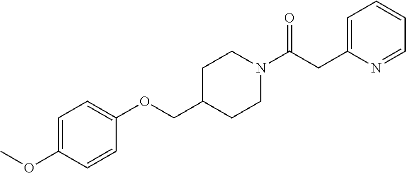 [SMILES: COc1ccc(OCC2CCN(C(=O)Cc3ccccn3)CC2)cc1] | 466 |
| 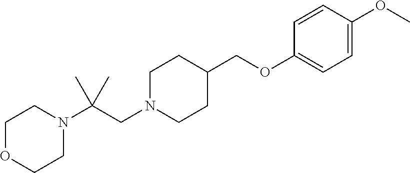 [SMILES: COc1ccc(OCC2CCN(CC(C)(C)N3CCOCC3)CC2)cc1] | 467 |
| 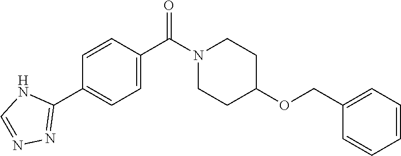 [SMILES: O=C(c1ccc(-c2nnc[nH]2)cc1)N1CCC(OCc2ccccc2)CC1] | 468 |
| 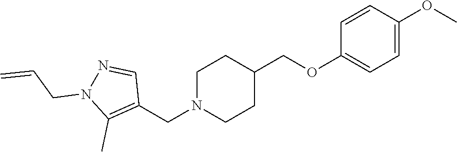 [SMILES: C=CCn1ncc(CN2CCC(COc3ccc(OC)cc3)CC2)c1C] | 469 |
| 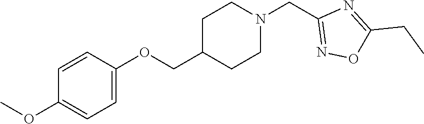 [SMILES: CCc1nc(CN2CCC(COc3ccc(OC)cc3)CC2)no1] | 470 |
| 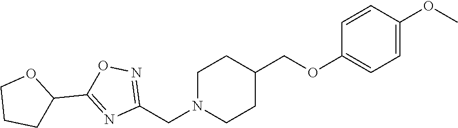 [SMILES: COc1ccc(OCC2CCN(Cc3noc(C4CCCO4)n3)CC2)cc1] | 471 |
| 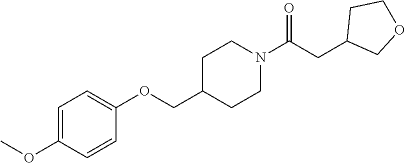 [SMILES: COc1ccc(OCC2CCN(C(=O)CC3CCOC3)CC2)cc1] | 472 |
| 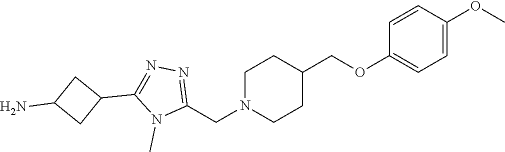 [SMILES: COc1ccc(OCC2CCN(Cc3nnc(C4CC(N)C4)n3C)CC2)cc1] | 473 |
| 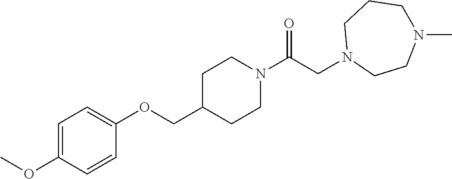 | 474 |
| 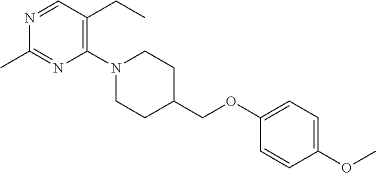 | 475 |
| 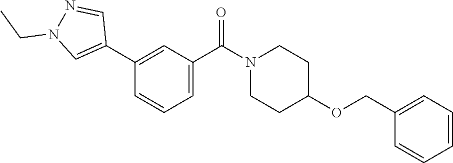 [SMILES: CCn1cc(-c2cccc(C(=O)N3CCC(OCc4ccccc4)CC3)c2)cn1] | 476 |
| 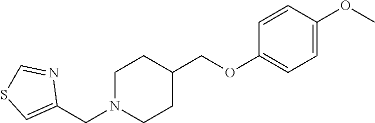 [SMILES: COc1ccc(OCC2CCN(Cc3cscn3)CC2)cc1] | 477 |
| 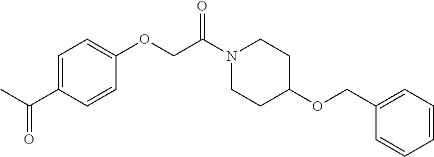 [SMILES: CC(=O)c1ccc(OCC(=O)N2CCC(OCc3ccccc3)CC2)cc1] | 478 |
| 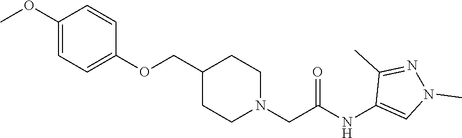 | 479 |
| 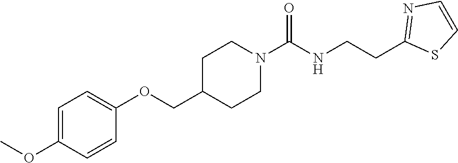 [SMILES: COc1ccc(OCC2CCN(C(=O)NCCc3nccs3)CC2)cc1] | 480 |
| 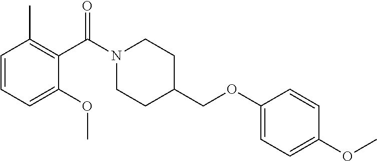 [SMILES: COc1ccc(OCC2CCN(C(=O)c3c(C)cccc3OC)CC2)cc1] | 481 |
| 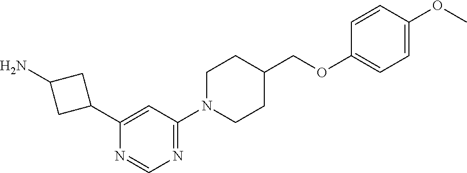 [SMILES: COc1ccc(OCC2CCN(c3cc(C4CC(N)C4)ncn3)CC2)cc1] | 482 |
| 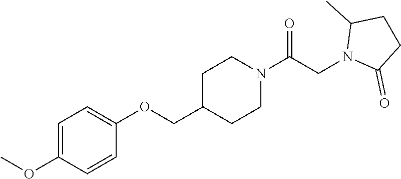 [SMILES: COc1ccc(OCC2CCN(C(=O)CN3C(=O)CCC3C)CC2)cc1] | 483 |
| 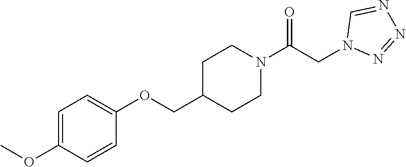 [SMILES: COc1ccc(OCC2CCN(C(=O)Cn3cnnn3)CC2)cc1] | 484 |
| 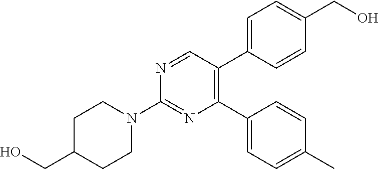 [SMILES: Cc1ccc(-c2nc(N3CCC(CO)CC3)ncc2-c2ccc(CO)cc2)cc1] | 485 |
| 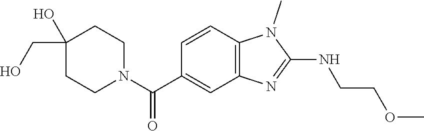 [SMILES: COCCNc1nc2cc(C(=O)N3CCC(O)(CO)CC3)ccc2n1C] | 486 |

In certain embodiments, the provided compounds (e.g., compounds of Formula (I)), activate GCase with an EC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

### Pharmaceutical Compositions, Kits, and Administration

The present disclosure provides pharmaceutical compositions comprising a disclosed compound (e.g., a compound of Formula (I)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease or disorder in a subject in need thereof. In certain embodiments, the effective amount is an amount effective for treating a neurological disease or disorder in a subject in need thereof. In certain embodiments, the effective amount is an amount effective for preventing a neurological disease or disorder in a subject in need thereof.

In certain embodiments, the effective amount is an amount effective for reducing the risk of developing a disease (e.g., neurological disease or disorder) in a subject in need thereof.

In certain embodiments, the effective amount is an amount effective for increasing the activity of GCase in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for increasing the activity of GCase by at least about 10%, at least about 20%, at least about 30%, at least about 40%, at least about 50%, at least about 60%, at least about 70%, at least about 80%, at least about 90%, at least about 95%, at least about 100%, at least about 150%, at least about 200%, at least about 250%, at least about 300%, at least about 400%, at least about 500%, or at least about 1000%. In certain embodiments, the effective amount is an amount effective for increasing the activity of GCase by a range between a percentage described in this paragraph and another percentage described in this paragraph, inclusive.

The present disclosure provides pharmaceutical compositions comprising a compound that interacts with (e.g., activates) GCase for use in treating a GCase-related disease or disorder in a subject in need thereof. The present disclosure provides pharmaceutical compositions comprising a compound that interacts with (e.g., activates) GCase for use in treating a disease or disorder associated with aberrant activity of GCase in a subject in need thereof. The present disclosure provides pharmaceutical compositions comprising a compound that interacts with (e.g., activates) GCase for use in treating a disease or disorder associated with mutated GCase in a subject in need thereof.

In certain embodiments, the composition is for use in treating a disease or disorder. In certain embodiments, the composition is for use in treating a neurological disease or disorder. In certain embodiments, the composition is for use in treating Gaucher's disease or Parkinson's disease. In certain embodiments, the composition is for use in treating Gaucher's disease. In certain embodiments, the composition is for use in treating Parkinson's disease.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disease or disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl, Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, cmu, cucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, car drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., neurological disease or disorder) in a subject in need thereof. In certain embodiments, the kits are useful for preventing a disease (e.g., neurological disease or disorder) in a subject in need thereof. In certain embodiments, the kits are useful for reducing the risk of developing a disease (e.g., neurological disease or disorder) in a subject in need thereof. In certain embodiments, the kits are useful for increasing the activity of GCase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological disease or disorder) in a subject in need thereof. In certain embodiments, the kits and instructions provide for preventing a disease (e.g., neurological disease or disorder) in a subject in need thereof. In certain embodiments, the kits and instructions provide for reducing the risk of developing a disease (e.g., neurological disease or disorder) in a subject in need thereof. In certain embodiments, the kits and instructions provide for increasing the activity of GCase in a subject or cell. A kit described herein may include one or more additional pharmaceutical agents described herein as a separate composition.

### Methods of Treatment

The present disclosure provides methods for treating a disease or disorder in a subject in need thereof. In certain embodiments, the present disclosure provides methods for treating a disease or disorder associated with GCase activity. In certain embodiments, the application provides a method of treating a neurological disease or disorder. In certain embodiments, the application provides a method of treating Gaucher's disease or Parkinson's disease. In certain embodiments, the application provides a method of treating Gaucher's disease. In certain embodiments, the application provides a method of treating Parkinson's disease.

The present disclosure provides a method of activating GCase. The present disclosure provides a method of increasing the activity of GCase. In certain embodiments, the application provides a method of activating GCase (e.g., increasing the activity of GCase) in vitro. In certain embodiments, the application provides a method of activating GCase (e.g., increasing the activity of GCase) in vivo. In certain embodiments, the application provides a method of increasing the activity of GCase in a cell. In certain embodiments, the application provides a method of increasing the activity of GCase in a human cell.

In certain embodiments, the methods comprise administering to a subject in need thereof (e.g., a subject with a neurological disease or disorder) a compound that interacts with GCase, for example, a compound that is a modulator of GCase (e.g., an activator of GCase), a binder of GCase, or a compound that modifies GCase. In certain embodiments, the methods comprise administering a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof.

Another object of the present disclosure is the use of a compound as described herein (e.g., of any formulae herein) in the manufacture of a medicament for use in the treatment of a disorder or disease described herein. Another object of the present disclosure is the use of a compound as described herein (e.g., of any formulae herein) for use in the treatment of a disorder or disease described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthetic Methods

Compounds of Formula (I) were prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P.G.M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art.

### General Procedure A

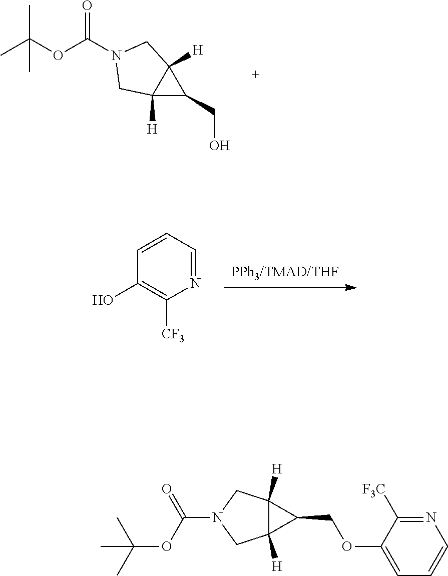 [SMILES: CC(C)(C)OC(=O)N1C[C@@H]2[C@@H](CO)[C@@H]2C1 ; Oc1cccnc1C(F)(F)F ; CC(C)(C)OC(=O)N1C[C@@H]2[C@@H](COc3cccnc3C(F)(F)F)[C@@H]2C1]

tert-butyl (1R,5S,6S)-6-({[2-(trifluoromethyl)pyridin-3-yl]oxy}methyl)-3-azabicyclo[3.1.0]hexane-3-carboxylate: To a stirred mixture of 2-(trifluoromethyl)pyridin-3-ol (191 mg, 1.17 mmol, 1.00 equiv) and tert-butyl (1R,5S,6r)-6-(hydroxymethyl)-3-azabicyclo[3.1.0]hexane-3-carboxylate (250 mg, 1.17 mmol, 1.00 equiv) in THF (10 mL) were added PPh₃ (492 mg, 1.88 mmol, 1.60 equiv) and TMAD (323 mg, 1.88 mmol, 1.6 equiv) in portions at 0° C. under nitrogen atmosphere. The resulting mixture was stirred overnight at room temperature under nitrogen atmosphere. The residue was purified by silica gel column chromatography, eluted with Hexane/EtOAc (3:1) to afford tert-butyl (1R,5S,6S)-6-({[2-(trifluoromethyl)pyridin-3-yl]oxy}methyl)-3-azabicyclo[3.1.0]hexane-3-carboxylate (390 mg, 92.8%) as an light-yellow oil. LCMS (ES, m/z): 359 [M+H]⁺.

### General Procedure B

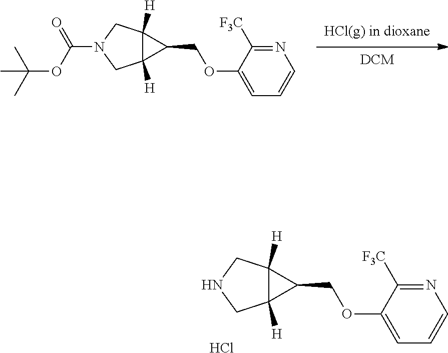

(1R,5S,6S)-6-({[2-(trifluoromethyl)pyridin-3-yl]oxy}methyl)-3-azabicyclo[3.1.0]hexane hydrochloride: To a stirred solution of tert-butyl (1R,5S,6S)-6-({[2-(trifluoromethyl)pyridin-3-yl]oxy}methyl)-3-azabicyclo[3.1.0]hexane-3-carboxylate (390 mg, 1.09 mmol, 1.00 equiv) in DCM (5 mL) was added HCl(gas) in 1,4-dioxane (4 M, 5.4 mL). The resulting mixture was stirred for 2 h at room temperature. The resulting mixture was concentrated to dryness under vacuum. The crude product (1R,5S,6S)-6-({[2-(trifluoromethyl)pyridin-3-yl]oxy}methyl)-3-azabicyclo[3.1.0]hexane hydrochloride (300 mg, 94%) was directly used next step without further purification. MS m/z: 259 [M+H]⁺.

### General Procedure C

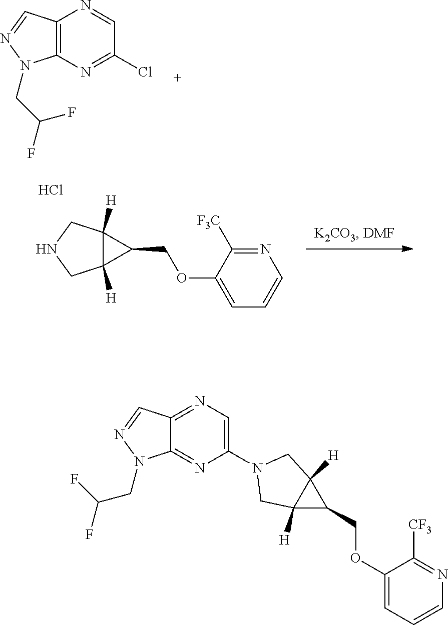 [SMILES: FC(F)Cn1ncc2ncc(Cl)nc21 ; Cl ; FC(F)(F)c1ncccc1OC[C@H]1[C@@H]2CNC[C@@H]21 ; FC(F)Cn1ncc2ncc(N3C[C@@H]4[C@@H](COc5cccnc5C(F)(F)F)[C@@H]4C3)nc21]

(1R,5S,6S)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane: To a stirred solution of (1R,5S,6S)-6-({[2-(trifluoromethyl)pyridin-3-yl]oxy}methyl)-3-azabicyclo[3.1.0]hexane hydrochloride (16.2 mg, 0.055 mmol, 1.00 equiv) and 6-chloro-1-(2,2-difluoroethyl)pyrazolo[3,4-b]pyrazine (12.0 mg, 0.055 mmol, 1.00 equiv) in DMF (1 mL) was added K₂CO₃ (15.2 mg, 0.11 mmol, 2 equiv). The resulting mixture was stirred at 60° C. for 16 h. The excessive solid was filtered off through Celite and the filtrate was concentrated under vacuum. The product was purified by silica gel column chromatography, eluted with Hexane/EtOAc (3:1 to 1:1), to afford (1R,5S,6S)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane as as a white powder (16 mg, 66.2%). MS m/z: 441.2 [M+H]⁺.

### General Procedure D

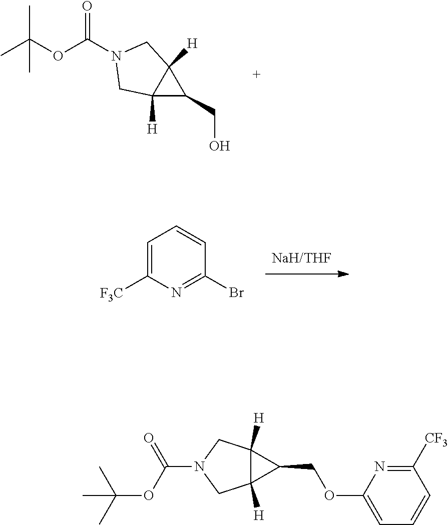 [SMILES: CC(C)(C)OC(=O)N1C[C@@H]2[C@@H](CO)[C@@H]2C1 ; FC(F)(F)c1cccc(Br)n1 ; CC(C)(C)OC(=O)N1C[C@@H]2[C@@H](COc3cccc(C(F)(F)F)n3)[C@@H]2C1]

tert-butyl (1R,5S,6S)-6-({[6-(trifluoromethyl)pyridin-2-yl]oxy}methyl)-3-azabicyclo[3.1.0]hexane-3-carboxylate: To a solution tert-butyl (1R,5S,6S)-6-(hydroxymethyl)-3-azabicyclo[3.1.0]hexane-3-carboxylate (250 mg, 1.17 mmol) in DMF (3.00 mL) was added NaH (51.6 mg, 1.1 eq., 60% w/w, 1.29 mmol) at 0° C., and allowed to warm to room temperature and stirred for 15 min. 2-bromo-6-(trifluoromethyl)pyridine (265 mg, 1.17 mmol) was added into the mixture, and the mixture was heated at 60° C. for 4 h. The reaction was monitored by LCMS. The mixture was diluted with water and extracted with EtOAc (20 mL×2). The organic layer was washed with brine, dried, filtered, evaporated, and purified by Combi-Flash to give tert-butyl (1R,5S,6S)-6-({[6-(trifluoromethyl)pyridin-2-yl]oxy}methyl)-3-azabicyclo[3.1.0]hexane-3-carboxylate (360 mg, 86) as a colorless oil.

### General Procedure E

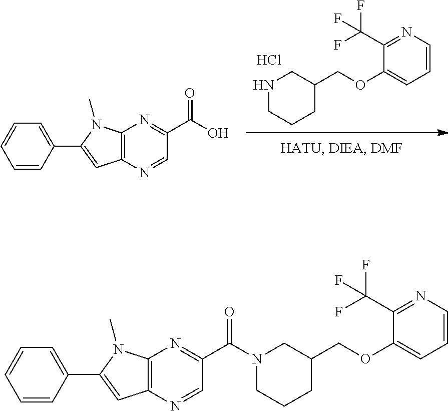 [SMILES: Cn1c(-c2ccccc2)cc2ncc(C(=O)O)nc21 ; Cl ; FC(F)(F)c1ncccc1OCC1CCCNC1 ; Cn1c(-c2ccccc2)cc2ncc(C(=O)N3CCCC(COc4cccnc4C(F)(F)F)C3)nc21]

(5-methyl-6-phenyl-5H-pyrrolo[2,3-b]pyrazin-3-yl)(3-(((2-(trifluoromethyl)pyridin-3-yl)oxy)methyl)piperidin-1-yl)methanone: To a stirred solution of 5-methyl-6-phenyl-5H-pyrrolo[2,3-b]pyrazine-3-carboxylic acid (100 mg, 0.395 mmol, 1.00 equiv) and HATU (165 mg, 0.435 mmol, 1.1 equiv) in DMF (1.5 mL) were added DIEA (204 mg, 1.58 mmol, 4 equiv) and 3-(piperidin-3-ylmethoxy)-2-(trifluoromethyl)pyridine hydrochloride (140 mg, 0.474 mmol, 1.2 equiv) dropwise at 0° C. The resulting mixture was stirred for 3 hours at 0° C. Desired product could be detected by LCMS. The reaction mixture was diluted by EtOAc (20 mL), washed by water (2×20 mL) and brine (1×20 mL), dried over anhydrous sodium sulfate. After filtration, the filtrate was concentrated. The residue was purified by reverse flash chromatography with the following conditions: column, C18 silica gel; mobile phase, ACN in water, 0% to 100% gradient in 30 min; detector, UV 254 nm. This provided (5-methyl-6-phenyl-5H-pyrrolo[2,3-b]pyrazin-3-yl)(3-(((2-(trifluoromethyl)pyridin-3-yl)oxy) methyl)piperidin-1-yl)methanone (60 mg, 29.3%) as a yellow solid. MS m/z: 496.2 [M+H]⁺

### (1R,5S,6S)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane (1)

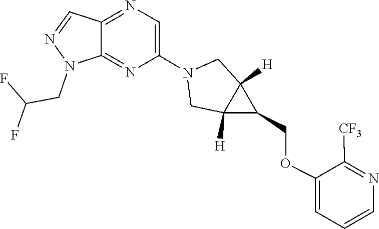 [SMILES: FC(F)Cn1ncc2ncc(N3C[C@@H]4[C@@H](COc5cccnc5C(F)(F)F)[C@@H]4C3)nc21]

Followed General Procedure C using (1R,5S,6S)-6-({[2-(trifluoromethyl)pyridin-3-yl]oxy}methyl)-3-azabicyclo[3.1.0]hexane hydrochloride (16.2 mg, 0.055 mmol, 1.00 equiv) and 6-chloro-1-(2,2-difluoroethyl)pyrazolo[3,4-b]pyrazine (12.0 mg, 0.055 mmol, 1.00 equiv to afford (1R,5S,6S)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexaneas as a white powder (16 mg, 66.2%). ¹H NMR (500 MHZ, CDCl₃) δ 8.26 (dd, J=4.6, 1.2 Hz, 1H), 8.02 (s, 1H), 7.91 (s, 1H), 7.43 (dd, J=8.5, 4.5 Hz, 1H), 7.34 (dd, J=8.3, 1.2 Hz, 1H), 6.21 (tt, J=55.7, 4.5 Hz, 1H), 4.64 (td, J=13.4, 4.5 Hz, 2H), 4.11 (d, J=6.2 Hz, 2H), 3.94 (d, J=10.8 Hz, 2H), 3.66 (dt, J=10.7, 2.1 Hz, 2H), 1.92 (td, J=3.2, 1.3 Hz, 2H), 1.23-1.20 (m, 1H). MS m/z: 441.2 [M+H]⁺.

### (1R,5S,6r)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane (2)

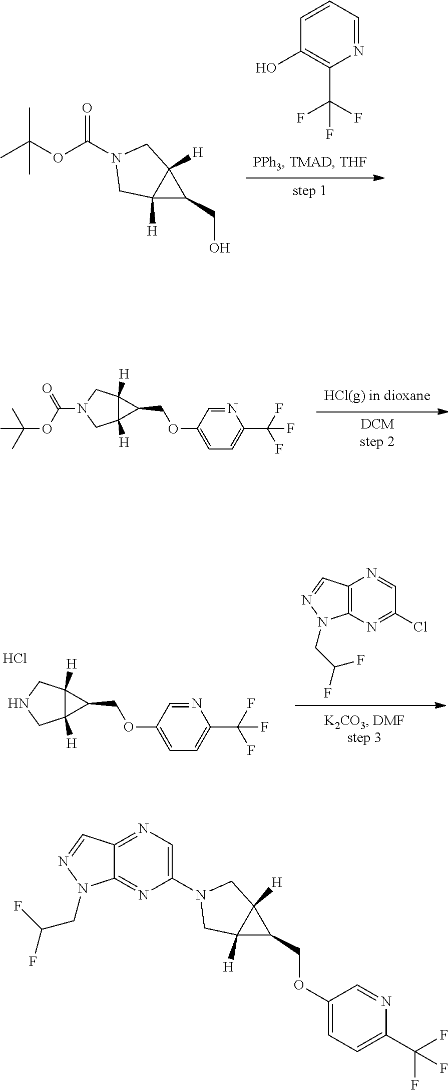

Step 1: tert-butyl (1R,5S,6r)-6-(((6-(trifluoromethyl)pyridin-3-yl)oxy)methyl)-3-azabicyclo[3.1.0]hexane-3-carboxylate: To a stirred mixture of 2-(trifluoromethyl)pyridin-3-ol (191 mg, 1.17 mmol, 1.00 equiv) and tert-butyl (1R,5S,6r)-6-(hydroxymethyl)-3-azabicyclo[3.1.0]hexane-3-carboxylate (250 mg, 1.17 mmol, 1.00 equiv) in THF (10 mL) were added PPh₃ (492 mg, 1.88 mmol, 1.60 equiv) and TMAD (323 mg, 1.88 mmol, 1.6 equiv) in portions at 0° C. under nitrogen atmosphere. The resulting mixture was stirred for overnight at room temperature under nitrogen atmosphere. The residue was purified by silica gel column chromatography, eluted with Hexane/EtOAc (3:1) to afford tert-butyl (1R,5S,6r)-6-(((6-(trifluoromethyl)pyridin-3-yl)oxy)methyl)-3-azabicyclo[3.1.0]hexane-3-carboxylate (390 mg, 92.8%) as an light-yellow oil. LCMS (ES, m/z): 359 [M+H]⁺.

Step 2: (1R,5S,6r)-6-(((6-(trifluoromethyl)pyridin-3-yl)oxy)methyl)-3-azabicyclo[3.1.0]hexane hydrochloride: To a stirred solution of tert-butyl (1R,5S,6r)-6-(((6-(trifluoromethyl)pyridin-3-yl)oxy)methyl)-3-azabicyclo[3.1.0]hexane-3-carboxylate (390 mg, 1.09 mmol, 1.00 equiv) in DCM (5 mL) was added HCl(gas) in 1,4-dioxane (4 M, 5.4 mL). The resulting mixture was stirred for 2 h at room temperature. The resulting mixture was concentrated to dryness under vacuum. The crude product (1R,5S,6r)-6-(((6-(trifluoromethyl)pyridin-3-yl)oxy)methyl)-3-azabicyclo[3.1.0]hexane hydrochloride (300 mg, 94%) was directly used in next step without further purification. MS m/z: 259 [M+H]⁺.

Step 3: (1R,5S,6r)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane: To a stirred solution of (1R,5S,6r)-6-(((6-(trifluoromethyl)pyridin-3-yl)oxy)methyl)-3-azabicyclo[3.1.0]hexane hydrochloride (16.2 mg, 0.055 mmol, 1.00 equiv) and 6-chloro-1-(2,2-difluoroethyl)pyrazolo[3,4-b]pyrazine (12.0 mg, 0.055 mmol, 1.00 equiv) in DMF (1 mL) was added K₂CO₃ (15.2 mg, 0.11 mmol, 2 equiv). The resulting mixture was stirred at 60° C. for 16 hours. The reaction mixture was diluted with water (10 mL), and stirred at rt for 15 min. The formed solid was filtered, washed with water, dried to give 16 mg (66.2%) (1R,5S,6r)-3-[1-(2,2-difluoroethyl)-1H-pyrazolo[3,4-b]pyrazin-6-yl]-6-({[2-(trifluoromethyl)pyridin-3-yl]oxy}methyl)-3-azabicyclo[3.1.0]hexane as a white powder. ¹H NMR (500 MHZ, CDCl₃) δ 8.26 (dd, J=4.6, 1.2 Hz, 1H), 8.02 (s, 1H), 7.91 (s, 1H), 7.43 (dd, J=8.5, 4.5 Hz, 1H), 7.34 (dd, J=8.3, 1.2 Hz, 1H), 6.21 (tt, J=55.7, 4.5 Hz, 1H), 4.64 (td, J=13.4, 4.5 Hz, 2H), 4.11 (d, J=6.2 Hz, 2H), 3.94 (d, J=10.8 Hz, 2H), 3.66 (dt, J=10.7, 2.1 Hz, 2H), 1.92 (td, J=3.2, 1.3 Hz, 2H), 1.23-1.20 (m, 1H). MS m/z: 441.2 [M+H]⁺.

### (3-(phenoxymethyl)piperidin-1-yl)(5-(1-phenylcyclopentyl)-1,3,4-oxadiazol-2-yl)methanone (3)

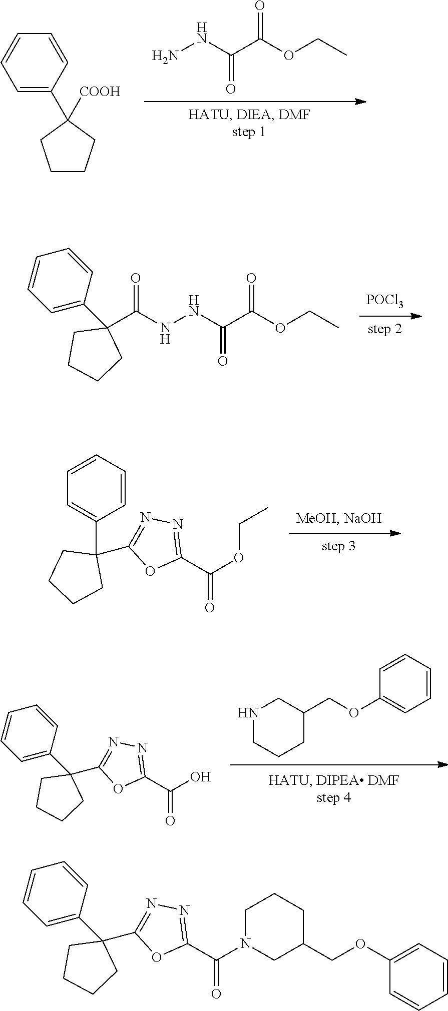

Step 1: ethyl 3-(2-benzoylhydrazineyl)-3-oxopropanoate: To a stirred solution of ethyl (hydrazinecarbonyl)formate (583 mg, 4.42 mmol, 1.20 equiv) and 1-phenylcyclopentane-1-carboxylic acid (700 mg, 3.68 mmol, 1.00 equiv) in DCM (12 mL) were added HATU (2.10 g, 5.52 mmol, 1.5 equiv) and DIPEA (713 mg, 5.52 mmol, 1.5 equiv) dropwise at 0° C. The resulting mixture was stirred for 3 hours at room temperature. The reaction was diluted with water (20 mL) and extracted with DCM (25 mL×2). The combined organic phases were washed with water (20 mL), brine (20 mL), dried over anhydrous Na₂SO₄, filtered and concentrated to dryness under vacuum to give the crude product. It was purified by chromatography on silica gel (Flash 40 g, 40-60% EtOAc:PE) to afford ethyl 2-oxo-2-(2-(1-phenylcyclopentane-1-carbonyl)hydrazineyl)acetate (800 mg, 60.7%) as a light yellow oil. MS m/z: 305[M+H]⁺.

Step 2: ethyl 5-(1-phenylcyclopentyl)-1,3,4-oxadiazole-2-carboxylate: A solution of ethyl 2-oxo-2-(2-(1-phenylcyclopentane-1-carbonyl)hydrazineyl)acetate (800 mg, 2.629 mmol, 1.00 equiv) in POCl₃ (10.0 mL) was stirred for 2 hours at 100° C. The resulting mixture was concentrated to dryness under vacuum. The residue was purified by reverse flash chromatography with the following conditions: column, C18 silica gel; mobile phase, MeCN in water, 5% to 95% gradient in 15 min; detector, UV 254 nm. to afford ethyl 5-(1-phenylcyclopentyl)-1,3,4-oxadiazole-2-carboxylate (600 mg, 71.7%) as a white solid. MS m/z: 267[M+H]⁺.

Step 3: 5-(1-phenylcyclopentyl)-1,3,4-oxadiazole-2-carboxylic acid: To the solution of ethyl 5-(1-phenylcyclopentyl)-1,3,4-oxadiazole-2-carboxylate (450 mg, 1.572 mmol, 1.00 equiv) in MeOH (2 mL) was added NaOH (96.6 mg, 2.41 mmol, 3 equiv) in water (1.00 mL). The mixture was stirred at room temperature for 1 h. The resulting mixture was concentrated to dryness under vacuum. And the resulting mixture was purified by reverse flash chromatography with the following conditions: column, C18 silica gel; mobile phase, MeCN in water, 5% to 95% gradient in 15 min; detector, UV 254 nm. to afford 5-(1-phenylcyclopentyl)-1,3,4-oxadiazole-2-carboxylic acid (300 mg) as a white solid. MS m/z: 259[M+H]⁺.

Step 4: (3-(phenoxymethyl)piperidin-1-yl)(5-(1-phenylcyclopentyl)-1,3,4-oxadiazol-2-yl)methanone: To a stirred solution of 5-(1-phenylcyclopentyl)-1,3,4-oxadiazole-2-carboxylic acid (150 mg, 0.581 mmol, 1.00 equiv) and 3-(phenoxymethyl)piperidine (133 mg. 0.697 mmol, 1.2 equiv) in DMF (2 mL) were added HATU (331 mg, 0.871 mmol, 1.5 equiv) and DIPEA (112 mg, 0.871 mmol, 1.5 equiv) dropwise at 0° C. The resulting mixture was stirred for additional 3 h at room temperature. The reaction mixture was diluted with water (10 mL), extracted with EtOAc (15 mL×2). The combined organic layers were washed with brine (3×10 mL), dried over anhydrous Na₂SO₄. After filtration, the filtrate was concentrated to dryness under reduced pressure. The residue was purified by reverse-phase Combi-Flash with the following conditions: column, C18 gel; mobile phase, MeCN in water (0.1% FA), 20% to 70% gradient in 16 min; detector, UV 254 nm. This provided (3-(phenoxymethyl)piperidin-1-yl)(5-(1-phenylcyclopentyl)-1,3,4-oxadiazol-2-yl)methanone (30.0 mg, 11.49%) as a white solid. ¹H NMR (400 MHZ, DMSO-d₆) δ 8.61-8.50 (m, 2H), 8.36-8.32 (m, 1H), 8.15 (s, 1H), 8.13-8.04 (m, 1H), 6.60-6.27 (m, 1H), 4.75-4.66 (m, 3H), 4.38-4.35 (m, 1H), 3.71-3.54 (m, 2H), 3.43-3.38 (m, 1H), 2.30-2.27 (m, 1H), 2.10-1.96 (m, 1H), 1.94-1.82 (m, 1H), 1.73-1.70 (m, 1H). MS m/z: 432.2 [M+H]⁺.

### 2-(1,3,4-thiadiazol-2-yl)-6-[3-({[2-(trifluoromethoxy)pyridin-3-yl]oxy}methyl) piperidin-1-yl]pyrazine (4)

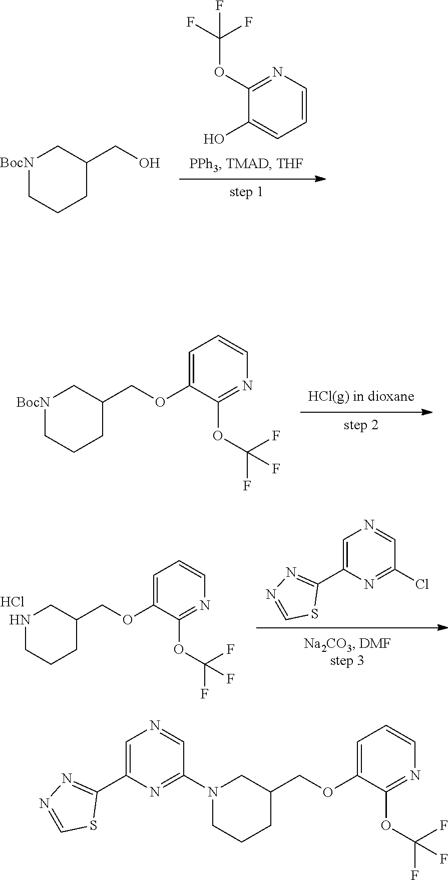

Step 1: tert-butyl 3-({[2-(trifluoromethoxy)pyridin-3-yl]oxy}methyl)piperidine-1-carboxylate: To a stirred mixture of 2-(trifluoromethoxy)pyridin-3-ol (250 mg, 1.40 mmol, 1.00 equiv) and tert-butyl 3-(hydroxymethyl)piperidine-1-carboxylate (300 mg, 1.40 mmol, 1.00 equiv) in THF (6 mL) were added PPh₃ (586 mg, 2.23 mmol, 1.60 equiv) and TMAD (384 mg, 2.23 mmol, 1.6 equiv) in portions at 0° C. under nitrogen atmosphere. The resulting mixture was stirred for overnight at room temperature under nitrogen atmosphere. The residue was purified by silica gel column chromatography, eluted with PE/EtOAc (1:1) to afford tert-butyl 3-({[2-(trifluoromethoxy)pyridin-3-yl]oxy}methyl)piperidine-1-carboxylate (460 mg, 87.6%) as an off-white solid. LCMS (ES, m/z): 377 [M+H]⁺.

Step 2: 3-(piperidin-3-ylmethoxy)-2-(trifluoromethoxy)pyridine hydrochloride: To a stirred solution of tert-butyl 3-({[2-(trifluoromethoxy)pyridin-3-yl]oxy}methyl)piperidine-1-carboxylate (460 mg, 1.22 mmol, 1.00 equiv) in DCM (8 mL) was added HCl(gas) in 1,4-dioxane (4 M, 4 mL.). The resulting mixture was stirred for 2 h at room temperature. The resulting mixture was concentrated to dryness under vacuum. The crude product 3-(piperidin-3-ylmethoxy)-2-(trifluoromethoxy)pyridine hydrochloride (380 mg) was directly used in next step without further purification. MS m/z: 277 [M+H]⁺.

Step 3: 2-(1,3,4-thiadiazol-2-yl)-6-[3-({[2-(trifluoromethoxy)pyridin-3-yl]oxy}methyl)-piperidin-1-yl]pyrazine: To a stirred mixture of 2-chloro-6-(1,3,4-thiadiazol-2-yl)pyrazine (40.0 mg, 0.201 mmol, 1.00 equiv) and 3-(piperidin-3-ylmethoxy)-2-(trifluoromethoxy)pyridine hydrochloride (75.6 mg, 0.241 mmol, 1.20 equiv) in DMF (3 mL) was added Na₂CO₃ (42.7 mg, 0.402 mmol, 2.00 equiv). The resulting mixture was stirred for 8 h at 80° C. The resulting mixture was diluted with water (15 mL), extracted with EtOAc (15 mL×3). The combined organic layers were washed with brine (2×20 mL), dried over anhydrous Na₂SO₄. After filtration, the filtrate was concentrated to dryness under reduced pressure. The residue was purified by reverse flash with the following conditions: column, C18 gel; mobile phase, MeCN in water, 30% to 70% gradient in 20 min; detector, UV 254 nm. This provided 2-(1,3,4-thiadiazol-2-yl)-6-[3-({[2-(trifluoromethoxy)pyridin-3-yl]oxy}methyl)piperidin-1-yl]pyrazine (45.1 mg, 51.1%) as a light yellow solid. ¹H NMR (300 MHz, DMSO-d₆): δ 9.73 (s, 1H), 8.59 (s, 1H), 8.48 (s, 1H), 7.91-7.86 (m, 1H), 7.78-7.70 (m, 1H), 7.45-7.36 (m, 1H), 4.48-4.35 (m, 1H), 4.31-4.18 (m, 1H), 4.16-4.04 (m, 2H), 3.23-3.01 (m, 2H), 2.21-2.06 (m, 1H), 2.00-1.88 (m, 1H), 1.87-1.76 (m, 1H), 1.70-1.39 (m, 2H). ¹⁹F NMR (282 MHZ, DMSO-d₆): δ −54.651. MS m/z: 439.10 [M+H]⁺.

### 1-(2,2-difluoroethyl)-6-((S)-3-((S)-1-(2-(trifluoromethyl)phenoxy)ethyl)piperidin-1-yl)-1H-pyrazolo[3,4-b]pyrazine (5a) and 1-(2,2-difluoroethyl)-6-((R)-3-((R)-1-(2-(trifluoromethyl)phenoxy)ethyl)piperidin-1-yl)-1H-pyrazolo[3,4-b]pyrazine (5b); trans-racemic-1-(2,2-difluoroethyl)-1-(2-(trifluoromethyl)phenoxy)ethyl)piperidin-1-yl)-1H-pyrazolo[3,4-b]pyrazine (5c)

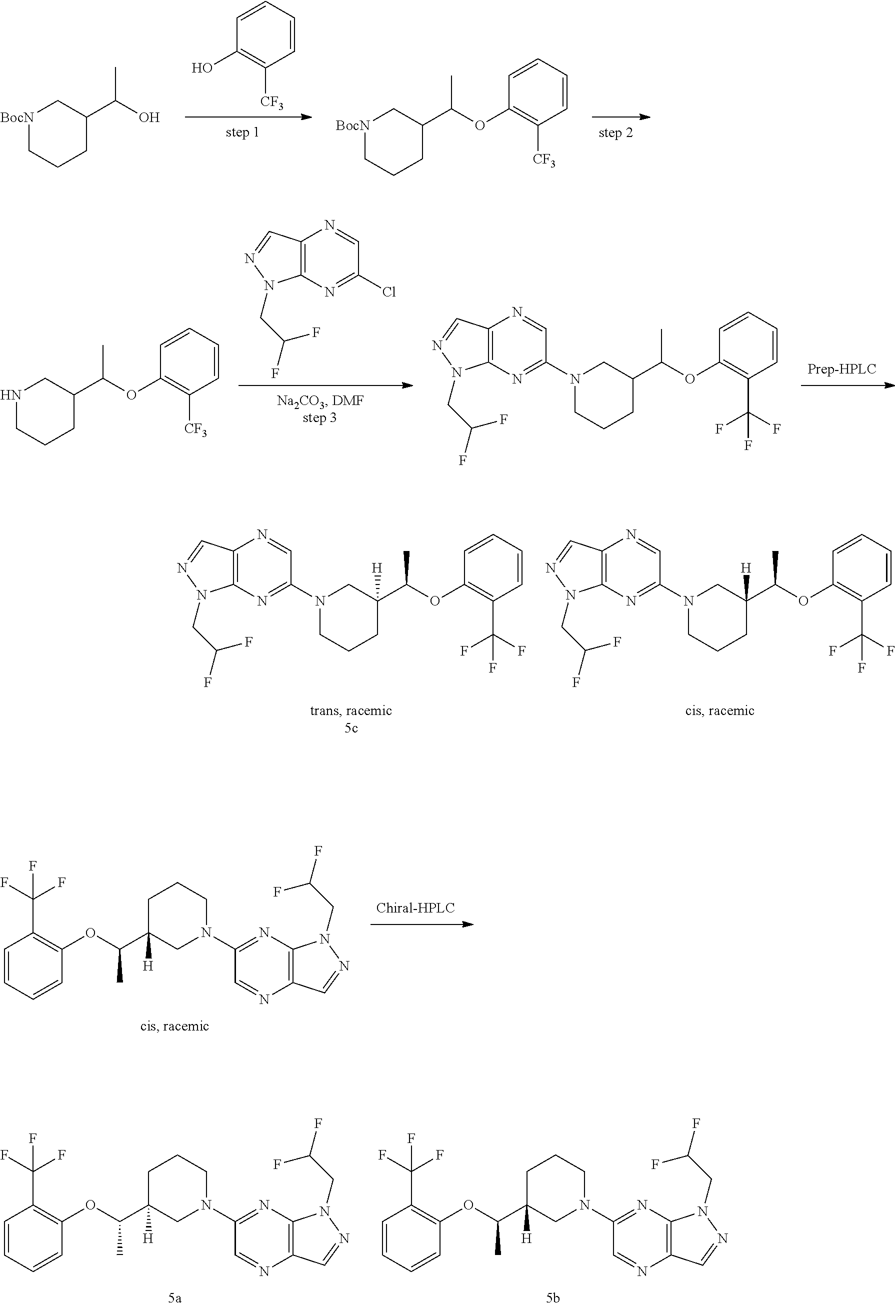

Step 1: tert-butyl 3-(1-(2-(trifluoromethyl)phenoxy)ethyl)piperidine-1-carboxylate: To a stirred mixture of tert-butyl 3-(1-hydroxyethyl)piperidine-1-carboxylate (1.00 g, 4.65 mmol, 1.00 equiv), 2-(trifluoromethyl)phenol (758 mg, 4.65 mmol, 1 equiv) and PPh₃ (1.95 g, 7.44 mmol, 1.6 equiv) in THF (10 mL) was added TMAD (1.28 g, 7.44 mmol, 1.6 equiv) in portions at 0° C. The resulting mixture was warmed to room temperature and stirred overnight at room temperature. The resulting mixture was concentrated to dryness under reduced pressure. The residue was purified by silica gel column chromatography, eluted with EtOAc/PE (1/2) to afford tert-butyl 3-(1-(2-(trifluoromethyl)phenoxy)ethyl)piperidine-1-carboxylate (600 mg, 49.7%) as a colorless oil. MS m/z: 318 [M-tBu+H]⁺.

Step 2: 3-(piperidin-3-ylmethoxy)-2-(trifluoromethyl)pyridine hydrochloride: To a stirred solution of tert-butyl 3-(1-(2-(trifluoromethyl)phenoxy)ethyl)piperidine-1-carboxylate (500 mg, 1.38 mmol, 1.00 equiv) in DCM (2.5 mL) was added HCl(gas) in dioxane (4 M, 2.5 mL). The mixture was stirred at room temperature for 2 h. After removing the solvent, the crude product 3-(1-(2-(trifluoromethyl)phenoxy)ethyl)piperidine hydrochloride (360 mg) was directly used in the next step without further purification MS m/z: 261 [M+H]⁺.

Step 3: 1-(2,2-difluoroethyl)-6-(3-(1-(2-(trifluoromethyl)phenoxy)ethyl)piperidin-1-yl)-1H-pyrazolo[3,4-b]pyrazine: To a stirred solution of 3-(1-(2-(trifluoromethyl)phenoxy)ethyl)piperidine (120 mg, 0.585 mmol, 1.00 equiv) and 6-chloro-1-(2,2-difluoroethyl)-1H-pyrazolo[3,4-b]pyrazine (153 mg, 0.702 mmol, 1.2 equiv) in DMF (2 mL) was added Na₂CO₃ (381 mg, 1.17 mmol, 2 equiv) at 0° C. The resulting mixture was stirred for 2 h at 100° C. The reaction mixture was purified by reverse flash chromatography with the following conditions: column, C18 silica gel; mobile phase, ACN in water, 0% to 100% gradient in 30 min; detector, UV 254 nm. This provided 1-(2,2-difluoroethyl)-6-(3-(1-(2-(trifluoromethyl)phenoxy)ethyl)piperidin-1-yl)-1H-pyrazolo[3,4-b]pyrazine (150 mg, 24.2%) as a yellow oil. The mixture was purified by Prep-HPLC to afford trans racemic (40.5 mg. 27.3%, assumed structure) and cis racemic (5c, 80.0 mg, 53.3%, assumed structure). The cis racemic (80.0 mg, 53.3%, assumed structure) was purified by Chiral-HPLC to afford 1-(2,2-difluoroethyl)-6-((S)-3-((S)-1-(2-(trifluoromethyl)phenoxy)ethyl)piperidin-1-yl)-1H-pyrazolo[3,4-b]pyrazine (5a; 20.0 mg, 25.0%, assumed structure) as a colorless oil and 1-(2,2-difluoroethyl)-6-((R)-3-((R)-1-(2-(trifluoromethyl)phenoxy)ethyl)piperidin-1-yl)-1H-pyrazolo[3,4-b]pyrazine (5b; 20.0 mg, 25.0%) as a colorless oil. ¹H NMR (400 MHZ, DMSO-d₆) δ 8.42 (s, 1H), 8.11 (s, 1H), 7.62-7.58 (m, 2H), 7.32-7.29 (m, 2H), 7.08-7.04 (m, 1H), 6.54-6.26 (m, 1H), 4.74-4.47 (m, 5H), 3.02-2.96 (m, 2H), 1.95-1.81 (m, 3H), 1.30-1.29 (m, 3H). MS m/z: 456.2 [M+H]⁺.

### 3-({1-[1-(2,2-difluoroethyl)pyrazolo[3,4-b]pyrazin-6-yl]piperidin-3-yl}methoxy)-2-(trifluoromethyl)pyridine (13)

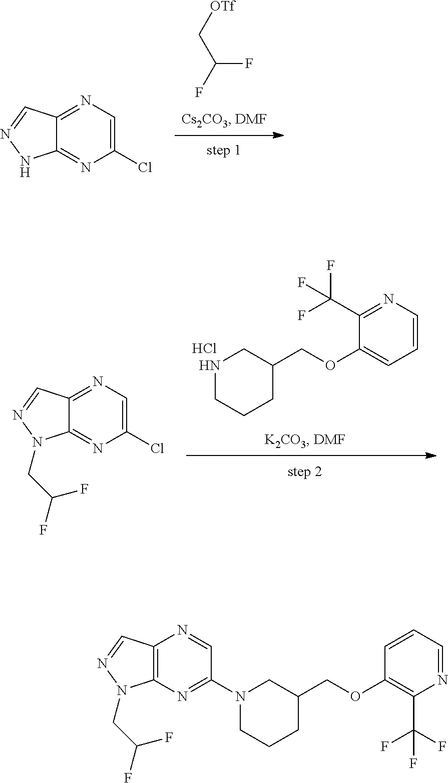 [SMILES: O=S(=O)(OCC(F)F)C(F)(F)F ; Clc1cnc2cn[nH]c2n1 ; FC(F)Cn1ncc2ncc(Cl)nc21 ; Cl ; FC(F)(F)c1ncccc1OCC1CCCNC1 ; FC(F)Cn1ncc2ncc(N3CCCC(COc4cccnc4C(F)(F)F)C3)nc21]

Step 1: 6-chloro-1-(2,2-difluoroethyl)pyrazolo[3,4-b]pyrazine: To a stirred mixture of 6-chloro-1H-pyrazolo[3,4-b]pyrazine (180 mg, 1.16 mmol, 1.00 equiv) and 2,2-difluoroethyl trifluoromethanesulfonate (373 mg, 1.75 mmol, 1.5 equiv) in DMF (2 mL) was added Cs₂CO₃ (1.14 g. 3.50 mmol, 3 equiv). The resulting mixture was stirred for 3 hours at room temperature. The reaction was diluted with water (20 mL) and extracted with EtOAc (20 mL×2). The combined organic phases were washed with water (40 mL), brine (40 mL), dried over anhydrous Na₂SO₄, filtered and concentrated to dryness under vacuum to give the crude product. It was purified by chromatography on silica gel (Flash 40 g, 40-60% EA:PE) to afford 6-chloro-1-(2,2-difluoroethyl)pyrazolo[3,4-b]pyrazine (130 mg, 51.1%) as a yellow solid. LCMS (ES, m/z): 219 [M+H]⁺.

Step 2: 3-({1-[1-(2,2-difluoroethyl)pyrazolo[3,4-b]pyrazin-6-yl]piperidin-3-yl}methoxy)-2-(trifluoromethyl)pyridine: To a stirred solution of 6-chloro-1-(2,2-difluoroethyl)pyrazolo[3,4-b]pyrazine (50.0 mg, 0.229 mmol, 1.00 equiv) and Cs₂CO₃ (223 mg, 0.687 mmol, 3 equiv) in DMF (1 mL) was added 3-(piperidin-3-ylmethoxy)-2-(trifluoromethyl)pyridine hydrochloride (81.4 mg, 0.275 mmol, 1.2 equiv). The resulting mixture was stirred for 4 hours at 80° C. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 3-({1-[1-(2,2-difluoroethyl)pyrazolo[3,4-b]pyrazin-6-yl]piperidin-3-yl}methoxy)-2-(trifluoro methyl)pyridine (20.0 mg, 19.8%) as a yellow solid. ¹H NMR (400 MHZ, DMSO-d₆) δ 8.43 (s, 1H), 8.27-8.26 (m, 1H), 8.12 (s, 1H), 7.83-7.81 (m, 1H), 7.71-7.68 (m, 1H), 6.55-6.25 (m, 1H), 4.78-4.56 (m, 3H), 4.41-4.37 (m 1H), 4.23-4.20 (m, 1H), 4.10-4.05 (m, 1H), 3.18-3.12 (m, 1H), 3.01-2.95 (m, 1H), 2.11 (s, 1H), 1.92-1.80 (m, 2H), 1.63-1.43 (m, 2H). MS m/z: 443.05 [M+H]⁺.

### 2-(6-(3-((o-tolyloxy)methyl)piperidin-1-yl)pyrazin-2-yl)-1,3,4-thiadiazole (14)

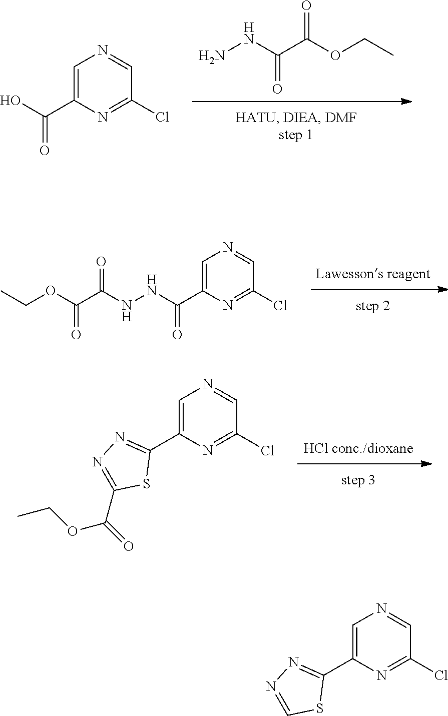

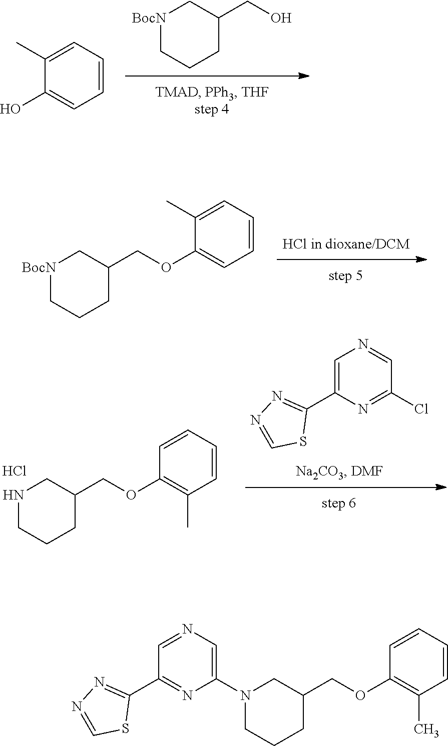

Step 1: ethyl 2-(2-(6-chloropyrazine-2-carbonyl)hydrazineyl)-2-oxoacetate: To a stirred solution of 6-chloropyrazine-2-carboxylic acid (2.00 g, 12.6 mmol, 1.0 eq.) and HATU (4.81 g, 12.6 mmol, 1.0 equiv) in DMF (20 mL) were added DIEA (4.76 g, 37.8 mmol, 3 equiv) and ethyl 2-hydrazineyl-2-oxoacetate (1.66 g, 12.6 mmol, 1.0 equiv) sequentially at 0° C. The resulting mixture was stirred for 3 hours at room temperature. The reaction was diluted with water (100 mL) and extracted with EtOAc (60 mL×2). The combined EtOAc phase was washed with water (100 mL), brine (100 mL), dried over anhydrous Na₂SO₄, filtered and concentrated under vacuum to give the crude product. It was purified by chromatography on silica gel (Flash 40 g, 40-60% EtOAc:PE) to afford ethyl 2-(2-(6-chloropyrazine-2-carbonyl)hydrazineyl)-2-oxoacetate (2.00 g, 58.3%) as a colorless oil. MS m/z: 273 [M+H]⁺.

Step 2: ethyl 5-(6-chloropyrazin-2-yl)-1,3,4-thiadiazole-2-carboxylate: A solution of ethyl 2-(2-(6-chloropyrazine-2-carbonyl)hydrazineyl)-2-oxoacetate (1.00 g, 3.67 mmol, 1 equiv.) and Lawesson Reagent (891 mg, 2.20 mmol, 0.6 equiv.) in toluene (10 mL) was stirred for 16 hours at 100° C. The reaction mixture was purified by chromatography on silica gel (Flash 40 g, 40-60% EtOAc:PE) to afford ethyl 5-(6-chloropyrazin-2-yl)-1,3,4-thiadiazole-2-carboxylate (460 mg, 46.4%) as a colorless oil. MS m/z: 271 [M+H]⁺.

Step 3: 2-(6-chloropyrazin-2-yl)-1,3,4-thiadiazole: To a stirred solution of ethyl 5-(6-chloropyrazin-2-yl)-1,3,4-thiadiazole-2-carboxylate (460 mg, 1.70 mmol, 1.00 equiv) in dioxane (5 mL) was added HCl conc. (1 mL) dropwise at room temperature. The resulting mixture was stirred for 2 hours at 100° C. The resulting mixture was concentrated to dryness under vacuum. The residue was purified by chromatography on silica gel (Flash 40 g, 40-60% EtOAc:PE) to afford 2-(6-chloropyrazin-2-yl)-1,3,4-thiadiazole (270 mg, 80.2%) as a white solid. MS m/z: 199 [M+H]⁺.

Step 4: tert-butyl 3-((o-tolyloxy)methyl)piperidine-1-carboxylate: To a stirred mixture of tert-butyl 3-(hydroxymethyl)piperidine-1-carboxylate (1.00 g, 4.65 mmol, 1.00 equiv), o-cresol (502 mg, 4.65 mmol, 1 equiv) and PPh₃ (1949 mg, 7.44 mmol, 1.6 equiv) in THF (10 mL) was added TMAD (1.280 g, 7.44 mmol, 1.6 equiv) in portion at 0° C. The resulting mixture was warmed to room temperature and stirred for overnight at room temperature. The resulting mixture was concentrated to dryness under reduced pressure. The residue was purified by silica gel column chromatography, eluted with EtOAc/PE (1/2) to afford tert-butyl 3-((o-tolyloxy)methyl)piperidine-1-carboxylate (1.00 g. 70.5%) as a colorless oil. MS m/z: 250 [M-tBu+H]⁺.

Step 5: 3-((o-tolyloxy)methyl)piperidine hydrochloride: tert-Butyl 3-((o-tolyloxy)methyl)piperidine-1-carboxylate (1.00 g, 3.27 mmol, 1.00 equiv) was dissolved in DCM (5 mL)/HCl(gas) in dioxane (4M, 5 mL). The mixture was stirred at room temperature for 1 h. After removing the solvent, the crude product 3-((o-tolyloxy)methyl)piperidine hydrochloride (750 mg) was directly used in next step without further purification MS m/z: 206 [M+H]⁺.

Step 6: 2-(6-(3-((o-tolyloxy)methyl)piperidin-1-yl)pyrazin-2-yl)-1,3,4-thiadiazole: To a stirred solution of 3-((o-tolyloxy)methyl)piperidine hydrochloride (90.0 mg, 0.425 mmol, 1 equiv) and 2-(6-chloropyrazin-2-yl)-1,3,4-thiadiazole (82.1 mg, 0.425 mmol, 1.00 equiv) in DMF (2 mL) was added Cs₂CO₃ (481 mg, 1.28 mmol, 3 equiv). The resulting mixture was stirred for 2 hours at 100° C. under nitrogen atmosphere. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 2-(6-(3-((o-tolyloxy)methyl)piperidin-1-yl)pyrazin-2-yl)-1,3,4-thiadiazole (31.0 mg, 19.7%) as a yellow green solid. ¹H NMR (300 MHz, DMSO-d₆) δ 9.73 (s, 1H), 8.59 (s, 1H), 8.51 (s, 1H), 7.19-7.07 (m, 2H), 6.93 (d, J=8.0 Hz, 1H), 6.89-6.78 (m, 1H), 4.63-4.53 (m, 1H), 4.28 (d, J=13.4 Hz, 1H), 4.05-3.84 (m, 2H), 3.19-3.06 (m, 1H), 3.00 (dd, J=13.1, 10.3 Hz, 1H), 2.25 (s, 3H), 2.16-2.03 (m, 1H), 1.98-1.87 (m, 1H), 1.87-1.75 (m, 1H), 1.67-1.39 (m, 2H). MS m/z: 368.15 [M+H]⁺.

### (3-(2-methylphenethyl)piperidin-1-yl)(2-phenyl-2H-1,2,3-triazol-4-yl)methanone (15)

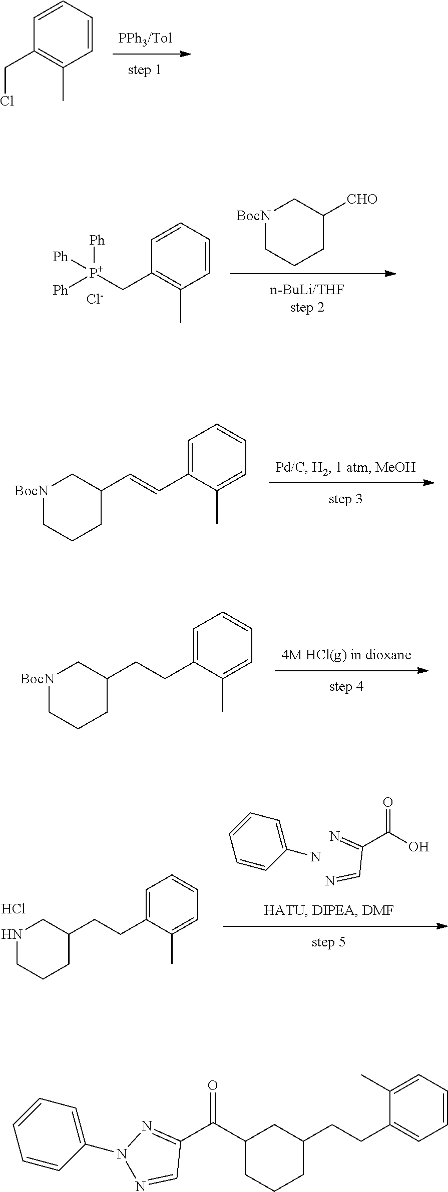

Step 1: (2-methylbenzyl)triphenylphosphonium: A solution of 1-(chloromethyl)-2-methylbenzene (500 mg, 3.6 mmol, 1 equiv.) and PPh₃ (1.0 g, 3.9 mmol, 1.1 equiv.) in toluene (15 mL) was stirred for 16 hours at 100° C. The reaction mixture was cooled to room temperature, then filtered, and the filter cake was washed with toluene (3×10 mL) to afford (2-methylbenzyl)triphenylphosphonium (1.01 g, 70.2%) as a white solid. MS m/z: 367[M+H]⁺.

Step 2: tert-butyl (E)-3-(2-methylstyryl)piperidine-1-carboxylate: To a stirred mixture of (2-methylbenzyl)triphenylphosphonium chloride (800.0 mg, 1.99 mmol, 1.20 equiv) in THF (20.00 mL) was added n-BuLi (2.5M in THF, 0.79 mL, 1.2 equiv) dropwise at −78° C. under N₂ atmosphere. The resulting mixture was allowed to warm to 0° C. and was stirred for 1 h at 0° C. under N₂ atmosphere. The reaction system was then cooled to −78° C. To the stirred solution was added tert-butyl 3-formylpiperidine-1-carboxylate (353.0 mg, 1.65 mmol, 1.00 equiv) in THF (1.00 mL) dropwise at −78° C. under N₂ atmosphere. The resulting mixture was allowed to warm to room temperature and was stirred for 8 h at room temperature under N₂ atmosphere. The reaction was quenched with saturated NH₄HCO₃ aq. at 0° C. The resulting mixture was extracted with EtOAc (3×10 mL). The combined organic layers were washed with brine (1×20 mL), dried over anhydrous Na₂SO₄. After filtration, the filtrate was concentrated under reduced pressure. The residue was purified by silica gel column chromatography, eluted with EtOAc/PE (1/3) to afford tert-butyl (E)-3-(2-methylstyryl)piperidine-1-carboxylate (350.0 mg, 70.0%) as a colorless oil. MS m/z: 302 [M+H]⁺.

Step 3: tert-butyl 3-(2-methylphenethyl)piperidine-1-carboxylate: To the solution of tert-butyl (E)-3-(2-methylstyryl)piperidine-1-carboxylate (350.0 mg, 1.1 mmol, 1.00 equiv) in MeOH (5.00 mL) was added Pt/C (10% w/w, 35.0 mg). The resulted mixture was hydrogenated overnight under H₂ (1 atm) atmosphere at room temperature. The reaction system was filtrated through celite and the filtrate was concentrated. The product tert-butyl 3-(2-methylphenethyl)piperidine-1-carboxylate (320 mg, 91.0%). MS m/z: 304 [M+H]⁺.

Step 4: 3-(2-methylphenethyl)piperidine hydrochloride: To a stirred solution of tert-butyl 3-(2-methylphenethyl)piperidine-1-carboxylate (300 mg, 0.99 mmol, 1.00 equiv) in DCM (4 mL) was added HCl (gas) in 1,4-dioxane (4M, 4 mL) dropwise at 0° C. The resulting mixture was stirred for 2 hours at room temperature. The resulting mixture was concentrated to dryness under vacuum. This provided 3-(2-methylphenethyl)piperidine hydrochloride (200 mg, 84.0%) as a white solid. MS m/z: 204.2[M+H]⁺. ¹H NMR (400 MHZ, DMSO-d₆) δ 9.04 (s, 1H), 8.81 (s, 1H), 7.13-7.05 (m, 4H), 3.35-3.17 (m, 2H), 2.74-2.53 (m, 4H), 2.25 (s, 3H), 1.89-1.75 (m, 4H), 1.48-1.19 (m, 3H). MS m/z: 204.2 [M+H]⁺.

Step 5: (3-(2-methylphenethyl)piperidin-1-yl)(2-phenyl-2H-1,2,3-triazol-4-yl)methanone: To a stirred solution of 2-phenyl-2H-1,2,3-triazole-4-carboxylic acid (21.6 mg, 0.11 mmol, 1.00 equiv) and HATU (47.7 mg, 0.12 mmol, 1.1 equiv) in DMF (2 mL) were added DIPEA (60 μL, 0.34 mmol, 3.0 equiv) and 3-(2-methylphenethyl)piperidine hydrochloride (27.5 mg, 0.11 mmol, 1.00 equiv) in sequence at room temperature. The resulting mixture was stirred for additional 16 h at room temperature. The reaction mixture was diluted with water (10 mL), extracted with EtOAc (15 mL×2). The combined organic layers were washed with brine (3×10 mL), dried over anhydrous Na₂SO₄. After filtration, the filtrate was concentrated to dryness under reduced pressure. The residue was purified by Combi-Flash silica gel column. This provided (3-(2-methylphenethyl)piperidin-1-yl)(2-phenyl-2H-1,2,3-triazol-4-yl)methanone (16.0 mg, 37.5%) as a colorless oil. ¹H NMR (500 MHz, DMSO-d₆) δ 8.38 (d, J=2.5 Hz, 1H), 8.03 (dd, J=8.0, 5.3 Hz, 2H), 7.60 (dt, J=11.4, 7.8 Hz, 2H), 7.48 (td, J=7.3, 5.1 Hz, 1H), 7.20-7.00 (m, 4H), 4.39 (d, J=13.0 Hz, 1H), 4.27 (dd, J=47.6, 13.3 Hz, 1H), 3.31-3.02 (m, 1H), 3.01-2.74 (m, 1H), 2.64 (t, J=7.9 Hz, 1H), 2.55 (q, J=7.1, 6.3 Hz, 1H), 2.23 (d, J=63.8 Hz, 3H), 1.95 (d, J=12.8 Hz, 1H), 1.75 (tt, J=13.3, 3.7 Hz, 1H), 1.69-1.27 (m, 5H). MS m/z: 375.3 [M+H]⁺.

### 1-{1-ethylpyrazolo[3,4-b]pyrazin-6-yl}-3-[2-(trifluoromethyl)phenoxymethyl]piperidine (16)

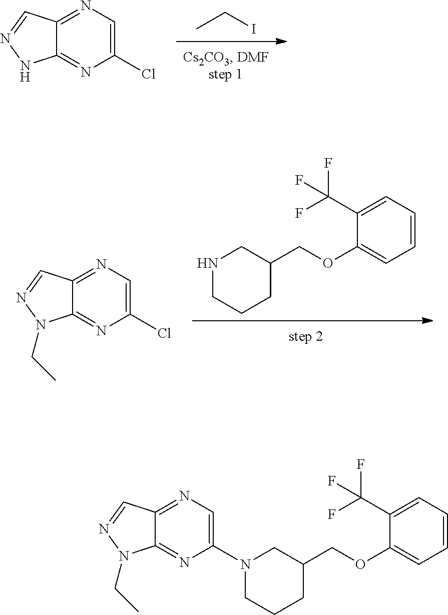

Step 1: 6-chloro-1-ethylpyrazolo[3,4-b]pyrazine: To a stirred solution of 6-chloro-1H-pyrazolo[3,4-b]pyrazine (300 mg, 1.94 mmol, 1.00 equiv) and cesium carbonate (1.27 mg, 3.88 mmol, 2 equiv) in DMF (4 mL) was added ethyl iodide (454.09 mg, 2.912 mmol, 1.5 equiv) dropwise at 0° C. The resulting mixture was stirred for 1 hour at room temperature. The reaction was diluted with water (20 mL) and extracted with EtOAc (25 mL×2). The combined organic phases were washed with water (20 mL), brine (20 mL), dried over anhydrous Na₂SO₄, filtered and concentrated to dryness under vacuum to give the crude product. It was purified by chromatography on silica gel (Flash 40 g, 40-60% EA:PE) to afford 6-chloro-1-ethylpyrazolo[3,4-b]pyrazine (320 mg, 90.4%) as a yellow solid. MS m/z: 183[M+H]⁺.

Step 2: 1-{1-ethylpyrazolo[3,4-b]pyrazin-6-yl}-3-[2-(trifluoromethyl)phenoxymethyl]piperidine: To a stirred solution of 3-[2-(trifluoromethyl)phenoxymethyl]piperidine (100 mg, 0.386 mmol, 1.00 equiv) and 6-chloro-1-ethylpyrazolo[3,4-b]pyrazine (84.5 mg, 0.463 mmol, 1.2 equiv) in DMF (1 mL) was added Cs₂CO₃ (251.33 mg, 0.772 mmol, 2 equiv). The resulting mixture was stirred for 3 hours at 80° C. The reaction mixture was purified by reverse flash chromatography with the following conditions: column, C18 silica gel; mobile phase, MeCN in water, 0% to 100% gradient in 30 min; detector, UV 254 nm. This provided 1-{1-ethylpyrazolo[3,4-b]pyrazin-6-yl}-3-[2-(trifluoromethyl)phenoxymethyl]piperidine (32.6 mg, 20.8%) as a yellow solid. LCMS (ES, m/z): 406.1 [M+H]⁺. ¹H NMR (400 MHZ, DMSO-d₆) δ 8.37 (s, 1H), 8.01 (s, 1H), 7.66-7.60 (m, 2H), 7.29-7.26 (m, 1H), 7.13-7.08 (m, 1H), 4.72-4.67 (m, 1H), 4.40-4.35 (m, 1H), 4.28-4.21 (m, 2H), 4.18-4.13 (m, 1H), 4.04-3.98 (m, 1H), 3.17-3.08 (m, 1H), 2.01-2.93 (m, 1H), 2.18-2.04 (m, 1H), 1.96-1.76 (m, 2H), 1.65-1.42 (m, 2H), 1.37-1.33 (m, 3H). MS m/z: 406.1 [M+H]⁺.

### 2-Phenyl-5-(3-(((2-(trifluoromethyl)pyridin-3-yl)oxy)methyl)piperidin-1-yl)-1,3,4-oxadiazole (17)

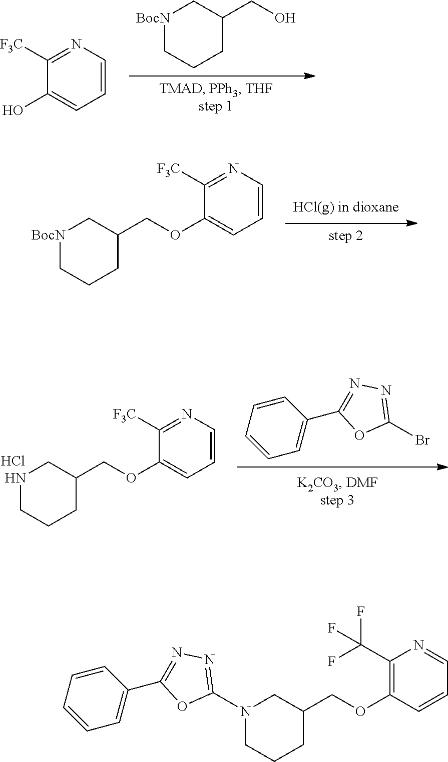

Step 1: tert-butyl 3-(((2-(trifluoromethyl)pyridin-3-yl)oxy)methyl)piperidine-1-carboxylate: To a stirred mixture of tert-butyl 3-(hydroxymethyl)piperidine-1-carboxylate (1.00 g, 4.65 mmol, 1.00 equiv), 2-(trifluoromethyl)pyridin-3-ol (758 mg, 4.65 mmol, 1 equiv) and PPh₃ (1.95 g, 7.44 mmol, 1.6 equiv) in THF (10 mL) was added TMAD (1.28 g, 7.44 mmol, 1.6 equiv) in portion at 0° C. The resulting mixture was warmed to room temperature and stirred overnight at room temperature. The resulting mixture was concentrated to dryness under reduced pressure. The residue was purified by silica gel column chromatography, eluted with EtOAc/PE (1/2) to afford tert-butyl 3-(((2-(trifluoromethyl)pyridin-3-yl)oxy) methyl) piperidine-1-carboxylate (1.00 g, 59.7%) as a colorless oil. MS m/z: 305 [M-tBu+H]⁺.

Step 2: 3-(piperidin-3-ylmethoxy)-2-(trifluoromethyl)pyridine hydrochloride: To a stirred solution of tert-butyl 3-(((2-(trifluoromethyl)pyridin-3-yl)oxy)methyl) piperidine-1-carboxylate (500 mg, 1.38 mmol, 1.00 equiv) in DCM (2.5 mL) was added HCl(gas) in dioxane (4M, 2.5 mL). The mixture was stirred at room temperature for 2 h. After removing the solvent, the crude product 3-(piperidin-3-ylmethoxy)-2-(trifluoromethyl) pyridine hydrochloride (360 mg) was directly used in next step without further purification MS m/z: 261 [M+H]⁺.

Step 3: 2-[5-(piperidin-3-yl)-1,3,4-thiadiazol-2-yl]-6-(trifluoromethyl)pyridine hydrochloride: To a stirred solution of 3-(piperidin-3-ylmethoxy)-2-(trifluoromethyl)pyridine hydrochloride (70.0 mg, 0.236 mmol, 1.00 equiv) and N,N-dimethyl-1,8-diazaspiro[4.5]decane-1-carboxamide (53.0 mg, 0.236 mmol, 1.00 equiv) in DMF (1.00 mL) was added K₂CO₃ (97.7 mg, 0.927 mmol, 3 equiv) at room temperature. The resulting mixture was stirred for 2 h at 100° C. under nitrogen atmosphere. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 2-phenyl-5-(3-(((2-(trifluoromethyl)pyridin-3-yl)oxy)methyl)piperidin-1-yl)-1,3,4-oxadiazole (32 mg, 33.6%) as an white solid. ¹H NMR (400 MHZ, DMSO-d₆) δ 8.26 (d, J=2.2 Hz, 1H), 7.90-7.79 (m, 3H), 7.75-7.65 (m, 1H), 7.59-7.47 (m, 1H), 4.26-4.17 (m, 1H), 4.15-4.03 (m, 2H), 3.83-3.94 (m, 1H), 3.21-3.02 (m, 2H), 2.23-2.10 (br, 1H), 1.95-1.74 (m, 2H), 1.72-1.55 (m, 1H), 1.50-1.35 (m, 1H). MS m/z: 405.1 [M+H]⁺.

### 1-[(oxetan-3-yl)methyl]-6-[(3S)-3-{2-[2-(trifluoromethyl)phenyl]ethyl}piperidine-1-carbonyl]-1H-indole (18)

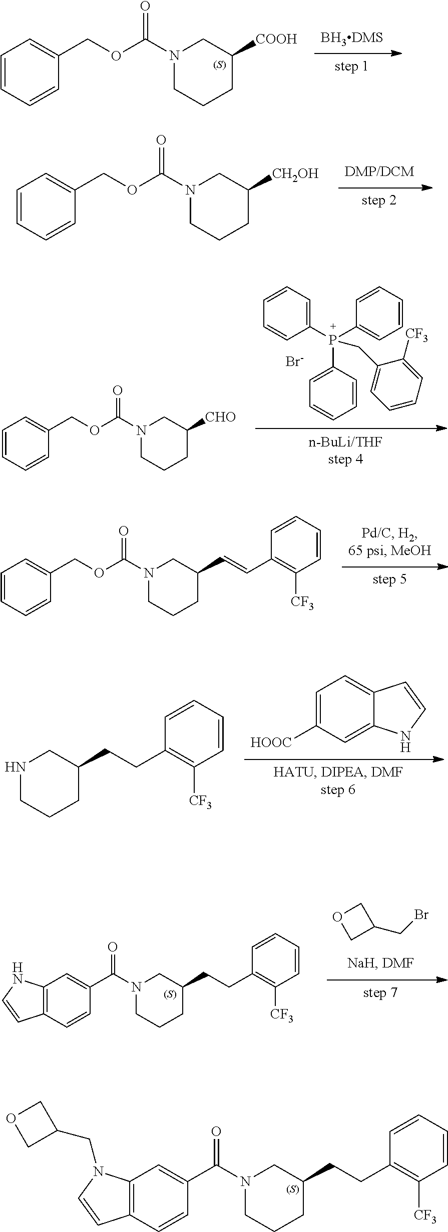

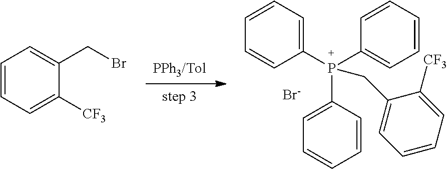

Step 1: benzyl (S)-3-(hydroxymethyl)piperidine-1-carboxylate: To a stirred solution of (S)-1-((benzyloxy)carbonyl)piperidine-3-carboxylic acid (1.50 g, 5.68 mmol, 1.00 equiv) in THF (15.0 mL) was added BH₃-Me₂S (1.7 mL, 4 M, 3.00 equiv) dropwise at 0° C. under N₂ atmosphere. The resulting mixture was stirred for 3 h at 0° C. under N₂ atmosphere. The reaction was quenched with water (30 mL) at 0° C. and extracted with EtOAc (3×20 mL). The combined organic layers were washed with brine (50 mL), dried over anhydrous Na₂SO₄. After filtration, the filtrate was concentrated under reduced pressure. The residue was purified by silica gel column chromatography, eluted with EtOAc/PE (2/1) to afford benzyl (S)-3-(hydroxymethyl)piperidine-1-carboxylate (1.0 g, 70.4%) as a colorless oil. MS m/z: 250 [M+H]⁺.

Step 2: benzyl (S)-3-formylpiperidine-1-carboxylate: To a stirred solution of benzyl (S)-3-(hydroxymethyl)piperidine-1-carboxylate (500 mg, 2.0 mmol, 1.00 equiv) in DCM (10 mL) was added Dess-Martin (1.0 g, 2.4 mmol, 1.2 equiv) in portions at 0 degrees C. The resulting mixture was stirred for 3 h at room temperature. The resulting mixture was filtered; the filter cake was washed with DCM (3×10 mL). The filtrate was concentrated under reduced pressure. The residue was purified by silica gel column chromatography, eluted with PE/EA (1:20) to afford benzyl (S)-3-formylpiperidine-1-carboxylate (400.0 mg, 80.1%) as a colorless oil. MS m/z: 248 [M+H]⁺.

Step 3: triphenyl(2-(trifluoromethyl)benzyl)phosphonium bromide: A solution of 1-(bromomethyl)-2-(trifluoromethyl)benzene (500 mg, 2.1 mmol, 1 equiv.) and PPh₃ (608.0 mg. 2.3 mmol, 1.1 equiv.) in toluene (5 mL) was stirred for 16 hours at 100° C. The reaction mixture was cooled to room temperature and filtered, the filter cake was washed with toluene (3×10 mL) to afford triphenyl(2-(trifluoromethyl)benzyl) phosphonium bromide (900 mg, 81.0%) as a white solid. MS m/z: 421[M+H]⁺.

Step 4: benzyl (R,E)-3-(2-(trifluoromethyl)styryl)piperidine-1-carboxylate: To a stirred mixture of triphenyl(2-(trifluoromethyl)benzyl)phosphonium (300.0 mg, 0.65 mmol, 1.00 equiv) in THF (12.00 mL) was added n-BuLi (2.5 M in THF, 0.26 mL, 1 equiv) dropwise at −78° C. under N₂ atmosphere. The resulting mixture was allowed to warm to 0° C. and was stirred for 30 min at 0° C. under N₂ atmosphere. The reaction system was then cooled to −78° C. To the stirred solution was added benzyl (S)-3-formylpiperidine-1-carboxylate (163.0 mg, 0.65 mmol, 1.00 equiv) in THF (1.00 mL) dropwise at −78° C. under N₂ atmosphere. The resulting mixture was allowed to warm to room temperature and was stirred for 8 h at room temperature under N₂ atmosphere. The reaction was quenched with saturated NH₄HCO₃ aq. at ° C. The resulting mixture was extracted with EtOAc (3×10 mL).

The combined organic layers were washed with brine (1×20 mL), dried over anhydrous Na₂SO₄. After filtration, the filtrate was concentrated under reduced pressure. The residue was purified by silica gel column chromatography, eluted with EtOAc/PE (1/3) to afford benzyl (R,E)-3-(2-(trifluoromethyl)styryl)piperidine-1-carboxylate (100 mg, 40.0%) as a colorless oil. MS m/z: 390 [M+H]⁺.

Step 5: (S)-3-(2-(trifluoromethyl)phenethyl)piperidine: To the solution of benzyl (R,E)-3-(2-(trifluoromethyl)styryl)piperidine-1-carboxylate (100.00 mg, 0.333 mmol, 1.00 equiv) in MeOH (5.00 mL) was added Pd/C (20% w/w, 20.0 mg). The resulted mixture was hydrogenated overnight under H₂ atmosphere (1 atm) at room temperature. The reaction mixture was filtrated through celite and the filtrate was concentrated to dryness. The product (S)-3-(2-(trifluoromethyl)phenethyl)piperidine (90 mg, 90.0%) was used directly for next step. MS m/z: 258 [M+H]⁺.

Step 6: (S)-(1H-indol-6-yl)(3-(2-(trifluoromethyl)phenethyl)piperidin-1-yl)methanone: To a stirred solution of 1H-indole-6-carboxylic acid (50 mg, 0.31 mmol, 1.00 equiv) and (S)-3-(2-(trifluoromethyl)phenethyl)piperidine (79.8 mg, 0.31 mmol, 1.0 equiv) in DMF (2 mL) were added HATU (129 mg, 0.34 mmol, 1.1 equiv) and DIPEA (58.5 mg, 0.46 mmol, 1.5 equiv) dropwise at 0° C. The resulting mixture was stirred for additional 3 h at room temperature. The reaction mixture was diluted with water (10 mL), extracted with EtOAc (15 mL×2). The combined organic layers were washed with brine (3×10 mL), dried over anhydrous Na₂SO₄. After filtration, the filtrate was concentrated to dryness under reduced pressure. The residue was purified by reverse-phase Combi-Flash with the following conditions: column, C18 gel; mobile phase, MeCN in water (0.1% FA), 20% to 70% gradient in 16 min; detector, UV 254 nm. This provided (S)-(1H-indol-6-yl)(3-(2-(trifluoromethyl)phenethyl)piperidin-1-yl)methanone (30.0 mg, 24.1%) as a white solid. ¹H NMR (400 MHZ, DMSO-d₆) δ 11.25 (s, 1H), 7.64-7.45 (m, 3H), 7.44-7.38 (m, 4H), 7.00-6.98 (m, 1H), 6.47 (s, 1H), 4.30-3.60 (m, 2H), 3.34-2.67 (m, 4H), 1.92-1.89 (m, 1H), 1.61-1.68 (m, 2H), 1.44-1.20 (m, 3H). MS m/z: 401.2 [M+H]⁺.

Step 7: 1-[(oxetan-3-yl)methyl]-6-[(3S)-3-{2-[2-(trifluoromethyl)phenyl]ethyl}piperidine-1-carbonyl]-1H-indole: A mixture of 6-[(3S)-3-{2-[2-(trifluoromethyl)phenyl]ethyl}piperidine-1-carbonyl]-1H-indole (15.0 mg, 37.5 μmol, 1.00 equiv) in DMF (1.00 mL) was added sodium hydride 60% w/w (1.65 mg, 41.2 μmol, 1.1 eq.) at room temperature and stirred for 15 min. The mixture was added 3-(bromomethyl)oxetane (5.66 mg, 37.5 μmol, 1.0 equiv) dropwise. The mixture was stirred at room temperature for 16 hrs. The reaction was monitored by LCMS. The mixture was diluted with water (15 mL), and extracted with EtOAc (20 mL). The organic layer was washed with brine, dried, filtered, concentrated, and purified with Combi-Flash to give 1-[(oxetan-3-yl)methyl]-6-[(3S)-3-{2-[2-(trifluoromethyl)phenyl]ethyl}piperidine-1-carbonyl]-1H-indole (15 mg, 85.1%) as a light-yellow sticky oil. ¹H NMR (500 MHZ, DMSO-d₆) δ 7.74-7.30 (m, 7H), 7.01 (ddd, J=9.8, 8.0, 1.4 Hz, 1H), 6.48 (d, J=3.2 Hz, 1H), 4.60 (dd, J=7.8, 6.1 Hz, 2H), 4.52 (d, J=7.4 Hz, 2H), 4.40 (td, J=6.1, 1.4 Hz, 2H), 4.37-4.09 (m, 1H), 3.85-3.52 (m, 1H), 3.50-3.36 (m, 1H), 3.12-2.60 (m, 4H), 1.96-1.87 (m, 1H), 1.78-1.32 (m, 5H), 1.31-1.21 (m, 1H). MS m/z: 471.1 [M+H]⁺.

### 1-(oxetan-3-yl)-6-[(3S)-3-{2-[2-(trifluoromethyl)phenyl]ethyl}piperidine-1-carbonyl]-1H-indole (19)

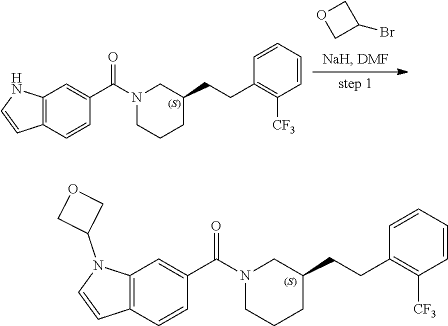

A mixture of 6-[(3S)-3-{2-[2-(trifluoromethyl)phenyl]ethyl}piperidine-1-carbonyl]-1H-indole (15.0 mg, 37.5 μmol, 1.00 equiv) in DMF (1.00 mL) was added sodium hydride 60% w/w (1.65 mg, 41.2 μmol, 1.1 eq.) at room temperature and stirred for 15 min. The mixture was added 3-bromooxetane (5.13 mg, 37.5 μmol, 1.0 equiv) dropwise. The mixture was stirred at room temperature for 16 hrs. The reaction was monitored by LCMS. The mixture was diluted with water (15 mL), and extracted with EtOAc (20 mL). The organic layer was washed with brine, dried, filtered, concentrated, and purified with Combi-Flash to give 1-(oxetan-3-yl)-6-[(3S)-3-{2-[2-(trifluoromethyl)phenyl]ethyl}piperidine-1-carbonyl]-1H-indole as light-yellow sticky oil (8.0 mg, 46.8%). ¹H NMR (500 MHZ, DMSO-d₆) δ 7.87 (d, J=3.2 Hz, 1H), 7.71-7.30 (m, 6H), 7.07 (dd, J=8.1, 1.3 Hz, 1H), 6.62 (d, J=3.2 Hz, 1H), 5.89-5.76 (m, 1H), 5.05 (td, J=7.4, 3.3 Hz, 2H), 4.92 (q, J=6.1 Hz, 2H), 4.46-4.24 (m, 1H), 3.85-3.53 (m, 1H), 3.14-2.61 (m, 4H), 1.96-1.87 (m, 1H), 1.82-1.20 (m, 6H). MS m/z: 457.1 [M+H]⁺.

### 3-({1-[1-(2,2-difluoroethyl)-1H-pyrazolo[3,4-b]pyrazin-6-yl]-4,4-difluoropiperidin-3-yl}methoxy)-2-(trifluoromethyl)pyridine (20)

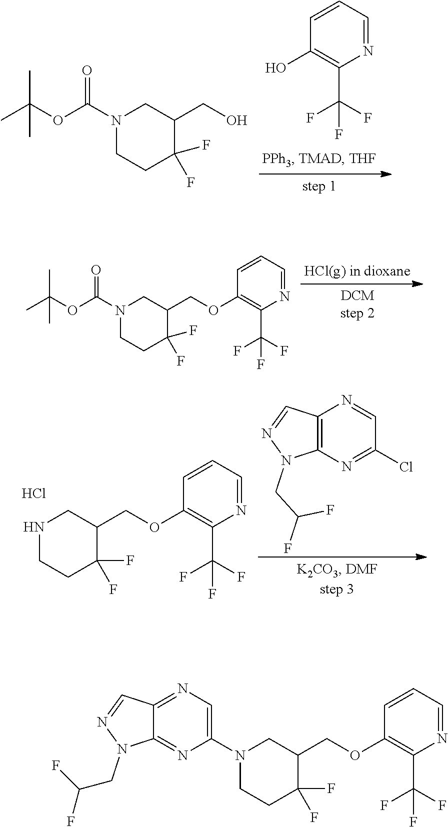

Step 1: tert-butyl 4,4-difluoro-3-(((2-(trifluoromethyl)pyridin-3-yl)oxy)methyl)piperidine-1-carboxylate: To a stirred mixture of 2-(trifluoromethyl)pyridin-3-ol (77.9 mg, 0.48 mmol, 1.00 equiv) and tert-butyl 4,4-difluoro-3-(hydroxymethyl)piperidine-1-carboxylate (120 mg, 0.48 mmol, 1.00 equiv) in THF (3 mL) were added PPh₃ (200 mg, 0.76 mmol, 1.60 equiv) and TMAD (132 mg, 0.76 mmol, 1.6 equiv) in portions at 0° C. under nitrogen atmosphere. The resulting mixture was stirred for overnight at room temperature under nitrogen atmosphere. The residue was purified by silica gel column chromatography, eluted with Hexane/EtOAc (4:1) to afford tert-butyl 4,4-difluoro-3-(((2-(trifluoromethyl)pyridin-3-yl)oxy)methyl)piperidine-1-carboxylate (100 mg, 52.8%) as an light-yellow oil. LCMS (ES, m/z): 397 [M+H]⁺.

Step 2: 3-((4,4-difluoropiperidin-3-yl)methoxy)-2-(trifluoromethyl)pyridine hydrochloride: To a stirred solution of tert-butyl 4,4-difluoro-3-(((2-(trifluoromethyl)pyridin-3-yl)oxy)methyl)piperidine-1-carboxylate (100 mg, 0.25 mmol, 1.00 equiv) in DCM (2 mL) was added HCl(gas) in 1,4-dioxane (4 M, 1.26 mL). The resulting mixture was stirred for 2 h at room temperature. The resulting mixture was concentrated to dryness under vacuum. The crude product 3-((4,4-difluoropiperidin-3-yl)methoxy)-2-(trifluoromethyl)pyridine hydrochloride (80 mg) was directly used in next step without further purification. MS m/z: 297 [M+H]⁺.

Step 3: 3-({1-[1-(2,2-difluoroethyl)-1H-pyrazolo[3,4-b]pyrazin-6-yl]-4,4-difluoropiperidin-3-yl}methoxy)-2-(trifluoromethyl)pyridine: To a stirred solution of 3-((4,4-difluoropiperidin-3-yl)methoxy)-2-(trifluoromethyl)pyridine hydrochloride (18.3 mg, 0.055 mmol, 1.00 equiv) and 6-chloro-1-(2,2-difluoroethyl)pyrazolo[3,4-b]pyrazine (12.0 mg, 0.055 mmol. 1.00 equiv) in DMF (1 mL) was added K₂CO₃ (15.2 mg, 0.11 mmol, 2 equiv). The resulting mixture was stirred at 60° C. for 16 hours. The reaction mixture was diluted with water (10 mL) and extracted with EtOAc (15 mL×2). The organic layer was evaporated and purified with Combi-flash (4 g silica gel column), eluted with Hex/EtOAc (1/1). The fractions were collected, and concentrated to give 4 mg (15.2%) 3-({1-[1-(2,2-difluoroethyl)-1H-pyrazolo[3,4-b]pyrazin-6-yl]-4,4-difluoropiperidin-3-yl}methoxy)-2-(trifluoromethyl)pyridine as a white powder. ¹H NMR (500 MHZ, CDCl₃) δ 8.30 (d, J=5.1 Hz, 2H), 8.06 (s, 1H), 7.47 (dd, J=8.5, 4.5 Hz, 1H), 7.40 (d, J=8.5 Hz, 1H), 6.17 (tt, J=55.4, 4.4 Hz, 1H), 4.88 (dt, J=14.2, 4.9 Hz, 1H), 4.71-4.63 (m, 2H), 4.56 (dd, J=9.2, 3.6 Hz, 1H), 4.47-4.40 (m, 1H), 4.08 (t, J=9.6 Hz, 1H), 3.48 (ddd, J=14.4, 11.7, 3.3 Hz, 1H), 3.33-3.25 (m, 1H), 2.74-2.59 (m, 1H), 2.31-2.20 (m, 1H), 2.16-2.00 (m, 1H). MS m/z: 479.2 [M+H]⁺.

### 3-({1-[1-(2,2-difluoroethyl)-1H-pyrazolo[3,4-b]pyrazin-6-yl]-5,5-difluoropiperidin-3-yl}methoxy)-2-(trifluoromethyl)pyridine (21)

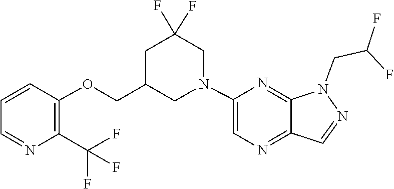 [SMILES: FC(F)Cn1ncc2ncc(N3CC(COc4cccnc4C(F)(F)F)CC(F)(F)C3)nc21]

Step 1: tert-butyl 3,3-difluoro-5-({[2-(trifluoromethyl)pyridin-3-yl]oxy}methyl)piperidine-1-carboxylate: Followed General Procedure A using tert-butyl 3,3-difluoro-5-(hydroxymethyl)piperidine-1-carboxylate (120 mg, 0.48 mmol, 1.00 equiv) and 2-(trifluoromethyl)pyridin-3-ol (78 mg, 0.48 mmol, 1.00 equiv) to afford tert-butyl 3,3-difluoro-5-({[2-(trifluoromethyl)pyridin-3-yl]oxy}methyl)piperidine-1-carboxylate (137 mg, 52.8%) as an light-yellow oil. MS m/z: 397 [M+H]⁺.

Step 2: 3-[(5,5-difluoropiperidin-3-yl)methoxy]-2-(trifluoromethyl)pyridine hydrochloride: Followed General Procedure B using tert-butyl 3,3-difluoro-5-({[2-(trifluoromethyl)pyridin-3-yl]oxy}methyl)piperidine-1-carboxylate (120 mg, 0.3 mmol, 1.00 equiv) to afford 3-[(5,5-difluoropiperidin-3-yl)methoxy]-2-(trifluoromethyl)pyridine hydrochloride (95 mg). MS m/z: 297 [M+H]⁺.

Step 3: 3-({1-[1-(2,2-difluoroethyl)-1H-pyrazolo[3,4-b]pyrazin-6-yl]-5,5-difluoropiperidin-3-yl}methoxy)-2-(trifluoromethyl)pyridine: Followed General Procedure C using 3-[(5,5-difluoropiperidin-3-yl)methoxy]-2-(trifluoromethyl)pyridine hydrochloride (18.3 mg, 0.055 mmol, 1.00 equiv) and 6-chloro-1-(2,2-difluoroethyl)pyrazolo[3,4-b]pyrazine (12.0 mg, 0.055 mmol, 1.00 equiv) to afford 3-({1-[1-(2,2-difluoroethyl)-1H-pyrazolo[3,4-b]pyrazin-6-yl]-5,5-difluoropiperidin-3-yl}methoxy)-2-(trifluoromethyl)pyridine as a white powder (4 mg, 15.2%). ¹H NMR (500 MHz, CDCl₃) δ 8.32 (d, J=4.7 Hz, 1H), 8.29 (s, 1H), 8.08 (d, J=1.0 Hz, 1H), 7.49 (dd, J=8.5, 4.6 Hz, 1H), 7.37 (d, J=8.5 Hz, 1H), 6.18 (tt, J=55.5, 4.4 Hz, 1H), 4.67 (td, J=13.4, 4.4 Hz, 2H), 4.56 (d, J=14.0 Hz, 1H), 4.50-4.41 (m, 1H), 4.18 (dd, J=9.1, 4.1 Hz, 1H), 4.06 (t, J=8.1 Hz, 1H), 3.66 (ddd, J=25.0, 14.1, 3.0 Hz, 1H), 3.41 (dd, J=13.7, 9.4 Hz, 1H), 2.63 (s, 1H), 2.38 (q. J=11.2, 10.0 Hz, 1H), 2.21-2.08 (m, 1H). MS m/z: 479.2 [M+H]⁺.

### (2-(2-Fluorophenyl)-2H-1,2,3-triazol-4-yl)(3-(phenoxymethyl)piperidin-1-yl)methanone (22)

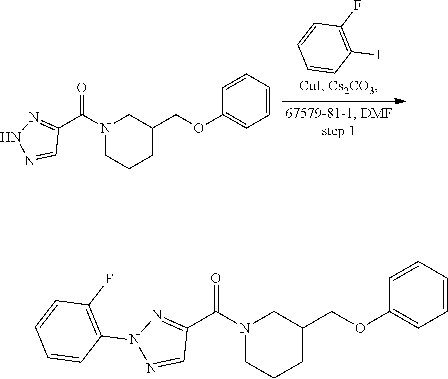

To a solution of 3-(phenoxymethyl)-1-(2H-1,2,3-triazole-4-carbonyl)piperidine (200 mg, 0.698 mmol, 1.00 equiv) and 1-fluoro-2-iodo-benzene (310 mg, 1.40 mmol, 2 equiv) in DMF (3 mL, 38.8 mmol, 55.5 equiv) were added CuI (13.3 mg, 0.070 mmol, 0.1 equiv), N1-(furan-2-ylmethyl)-N2-(2-methylnaphthalen-1-yl)oxalamide (21.5 mg, 0.070 mmol, 0.1 equiv) and Cs₂CO₃ (683 mg, 2.10 mmol, 3 equiv) under N₂ atmosphere. The result mixture was heated to 90° C. and stirred overnight. Desired product could be detected by LCMS. The reaction mixture was diluted by EtOAc (20 mL), washed by water (2×20 mL) and brine (1×20 mL), dried over anhydrous sodium sulfate. After filtration, the filtrate was concentrated. The residue was purified by silica gel column chromatography, eluted with EtOAc/PE=1/5 to afford impure product. The impure product was further purified by reverse flash chromatography with the following conditions: column, C18 silica gel; mobile phase, MeCN in water, 35% to 75% gradient in 15 min; detector, UV 254 nm. This provided (2-(2-fluorophenyl)-2H-1,2,3-triazol-4-yl)(3-(phenoxymethyl)piperidin-1-yl)methanone (17.2 mg, 6.47%) as a white solid. ¹H NMR (400 MHZ, DMSO-d₆) δ 8.51-8.25 (d, J=20.9 Hz, 1H), 7.69-7.24 (m, 1H), 7.59 (t, J=11.3 Hz, 2H), 7.51-7.37 (m, 1H), 7.30 (t, J=7.7 Hz, 1H), 7.19 (t, J=7.2 Hz, 1H), 7.02-6.83 (m, 2H), 6.81-6.79 (m, 1H), 4.62-4.33 (m, 1H), 4.27 (d, J=13.2 Hz, 1H), 4.07-3.72 (m, 2H), 3.27-3.18 (m, 1H), 3.12-2.78 (m, 1H), 2.13-1.96 (m, 1H), 1.97-1.86 (m, 1H), 1.84-1.69 (m, 1H), 1.66-1.33 (m, 2H). MS m/z: 381.2 [M+H]⁺.

### 2-(6-(3-((o-Tolyloxy)methyl)piperidin-1-yl)-1H-pyrazolo[3,4-b]pyrazin-1-yl)-1,3,4-thiadiazole (23)

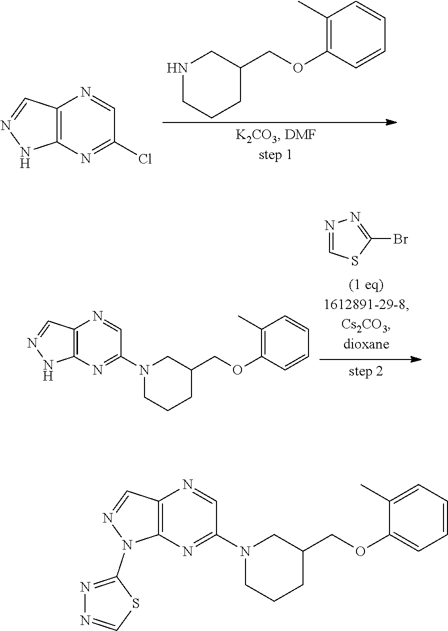 [SMILES: Clc1cnc2cn[nH]c2n1 ; Cc1ccccc1OCC1CCCNC1 ; Brc1nncs1 ; Cc1ccccc1OCC1CCCN(c2cnc3cn[nH]c3n2)C1 ; Cc1ccccc1OCC1CCCN(c2cnc3cnn(-c4nncs4)c3n2)C1]

Step 1: 6-(3-((o-tolyloxy)methyl)piperidin-1-yl)-1H-pyrazolo[3,4-b]pyrazine: To a stirred solution of 6-chloro-1H-pyrazolo[3,4-b]pyrazine (180 mg, 1.16 mmol, 1 equiv) and 3-((o-tolyloxy)methyl)piperidine (263 mg, 1.28 mmol, 1.1 equiv) in DMF (3 mL) was added K₂CO₃ (321 mg, 2.33 mmol, 2 equiv) at room temperature under air atmosphere. The resulting mixture was stirred for overnight at 100° C. under nitrogen atmosphere. The resulting mixture was diluted with EtOAc (30 mL). The combined organic layers were washed with water (2×20 mL) brine (1×10 mL), dried over anhydrous Na₂SO₄. After filtration, the filtrate was concentrated under reduced pressure. The residue was purified by silica gel column chromatography, eluted with PE/EA (5:1) to afford 6-(3-((o-tolyloxy)methyl)piperidin-1-yl)-1H-pyrazolo[3,4-b]pyrazine (120 mg, 31.8%) as an off-white solid. MS m/z: 324 [M+H]⁺.

Step 2: 2-(6-(3-((o-tolyloxy)methyl)piperidin-1-yl)-1H-pyrazolo[3,4-b]pyrazin-1-yl)-1,3,4-thiadiazole: To a stirred solution of 6-(3-((o-tolyloxy)methyl)piperidin-1-yl)-1H-pyrazolo[3,4-b]pyrazine (100 mg, 0.309 mmol, 1.00 equiv) and 2-bromo-1,3,4-thiadiazole (51.0 mg, 0.309 mmol, 1 equiv) in dioxane (2 mL) were added Cs₂CO₃ (201 mg, 0.618 mmol, 2 equiv), Pd-PEPPSI-IPentCl 2-methylpyridine (o-picoline (26.0 mg, 0.031 mmol, 0.1 equiv). The resulting mixture was stirred for 16 h at 100° C. under nitrogen atmosphere. Desired product could be detected by LCMS. The resulting mixture was diluted with water (10 mL), extracted with EtOAc (3×10 mL). The combined organic layers were dried over anhydrous Na₂SO₄. After filtration, the filtrate was concentrated under reduced pressure. The residue was purified by reverse flash chromatography with the following conditions: column. C18 silica gel; mobile phase, MeCN in water, 10% to 50% gradient in 10 min; detector, UV 254 nm. This provided 2-(6-(3-((o-tolyloxy)methyl)piperidin-1-yl)-1H-pyrazolo[3,4-b]pyrazin-1-yl)-1,3,4-thiadiazole (12.5 mg, 9.44%) MS m/z: 407.95 [M+H]⁺. ¹H NMR (300 MHz, DMSO-d₆) δ 9.48 (d, J=1.7 Hz, 1H), 8.63-8.47 (m, 2H), 7.21-7.07 (m, 2H), 6.93 (d, J=8.0 Hz, 1H), 6.87-6.76 (m, 1H), 4.73-4.38 (m, 2H), 3.98 (d, J=6.2 Hz, 2H), 3.21 (d, J=11.9 Hz, 2H), 2.22 (s, 3H), 2.11 (d, J=18.2 Hz, 1H), 2.01-1.80 (m, 2H), 1.60 (d, J=9.1 Hz, 2H).

### (3-(Phenoxymethyl)piperidin-1-yl)(5-(2-phenylpropan-2-yl)-1,3,4-oxadiazol-2-yl)methanone (24)

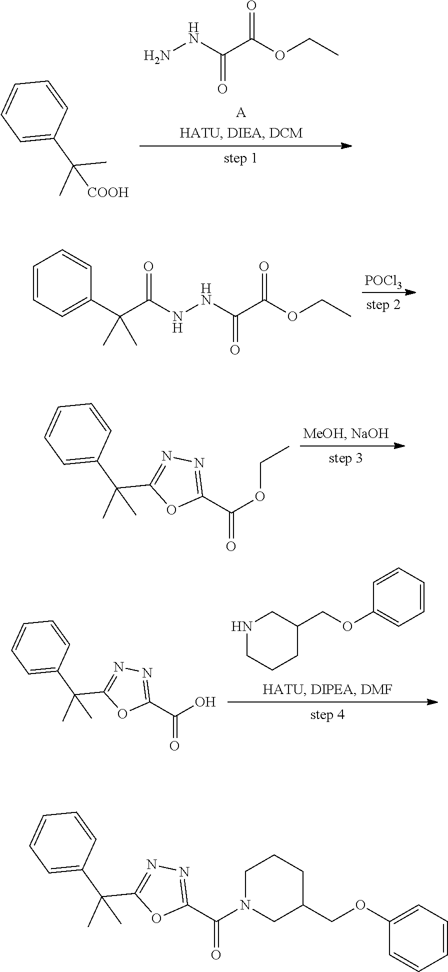

Step 1: ethyl 2-(2-(2-methyl-2-phenylpropanoyl)hydrazineyl)-2-oxoacetate: To a stirred solution of 2-methyl-2-phenylpropanoic acid (1 g, 6.09 mmol, 1.00 equiv) and HATU (2.55 g, 6.69 mmol, 1.1 equiv) in DCM (50 mL) were added DIEA (1.57 g, 12.1 mmol, 2 equiv) and ethyl 2-hydrazineyl-2-oxoacetate (0.97 g, 7.30 mmol, 1.2 equiv) in portions at 0° C. The resulting mixture was stirred for 16 hours at room temperature. The resulting mixture was concentrated under vacuum. The residue was purified by silica gel column chromatography, eluted with PE/EA (1/1) to afford ethyl 2-(2-(2-methyl-2-phenylpropanoyl)hydrazineyl)-2-oxoacetate (1.2 g, 70.8%) as a white solid. MS m/z: 279[M+H]⁺.

Step 2: ethyl 5-(2-phenylpropan-2-yl)-1,3,4-oxadiazole-2-carboxylate: A solution of ethyl 2-(2-(2-methyl-2-phenylpropanoyl)hydrazineyl)-2-oxoacetate (1 g, 3.59 mmol, 1.00 equiv) in phosphorus oxychloride (10 mL) was stirred for 2 hours at 100° C. The resulting mixture was diluted with EtOAc (100 mL). The organic layer was washed with Na₂CO₃ (100 mL), dried over anhydrous Na₂SO₄. After filtration, the filtrate was concentrated under reduced pressure. The residue was purified by silica gel column chromatography, eluted with PE/EA (1:2) to afford ethyl 5-(2-phenylpropan-2-yl)-1,3,4-oxadiazole-2-carboxylate (600 mg, 64.1%) as a light-yellow oil. MS m/z: 261 [M+H]⁺.

Step 3: 5-(2-phenylpropan-2-yl)-1,3,4-oxadiazole-2-carboxylic acid: A solution of ethyl 5-(2-phenylpropan-2-yl)-1,3,4-oxadiazole-2-carboxylate (220 mg, 0.845 mmol, 1.00 equiv) and NaOH (135 mg, 3.38 mmol, 4 equiv) in MeOH/H₂O (1 mL/1 mL) was stirred for 3 hours at room temperature. Desired product could be detected by LCMS. The mixture was acidified to pH 5 with HCl (1 mol/L). The resulting mixture was extracted with EtOAc (3×10 mL). The combined organic layers were washed with water (3×15 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 5-(2-phenylpropan-2-yl)-1,3,4-oxadiazole-2-carboxylic acid (140 mg, 71.3%) as a white solid. MS m/z: 233[M+H]⁺.

Step 4: (3-(phenoxymethyl)piperidin-1-yl)(5-(2-phenylpropan-2-yl)-1,3,4-oxadiazol-2-yl)methanone: To a stirred solution of 5-(2-phenylpropan-2-yl)-1,3,4-oxadiazole-2-carboxylic acid (60 mg, 0.258 mmol, 1.00 equiv) and HATU (108 mg, 0.284 mmol, 1.1 equiv) in DMF (1 mL) were added DIEA (66.7 mg, 0.516 mmol, 2 equiv) and 3-(phenoxymethyl)piperidine (59.3 mg, 0.310 mmol, 1.2 equiv) in portions at 0° C. The resulting mixture was stirred for 2 hours at room temperature. The residue was purified by reverse flash chromatography with the following conditions: column, C18 silica gel; mobile phase, MeCN in water, 10% to 100% gradient in 30 min; detector, UV 254 nm. This provided 3-(phenoxymethyl)piperidin-1-yl)(5-(2-phenylpropan-2-yl)-1,3,4-oxadiazol-2-yl)methanone (35 mg, 33.11%) as a white solid. ¹H NMR (400 MHZ, DMSO-d₆) δ 7.43-7.22 (m, 7H), 7.01-6.82 (m, 3H), 4.49-4.09 (m, 2H), 3.98-3.76 (m, 2H), 3.38-3.24 (m, 1H), 3.12-2.87 (m, 1H), 2.07-2.05 (m, 1H), 1.93-1.85 (m, 1H), 1.83-1.70 (m, 7H), 1.57-1.39 (m, 2H). MS m/z: 406.3 [M+H]⁺.

### (4-Chloro-7-phenylpyrazolo[1,5-a]pyridin-3-yl)(3-((o-tolyloxy)methyl)piperidin-1-yl)methanone (25)

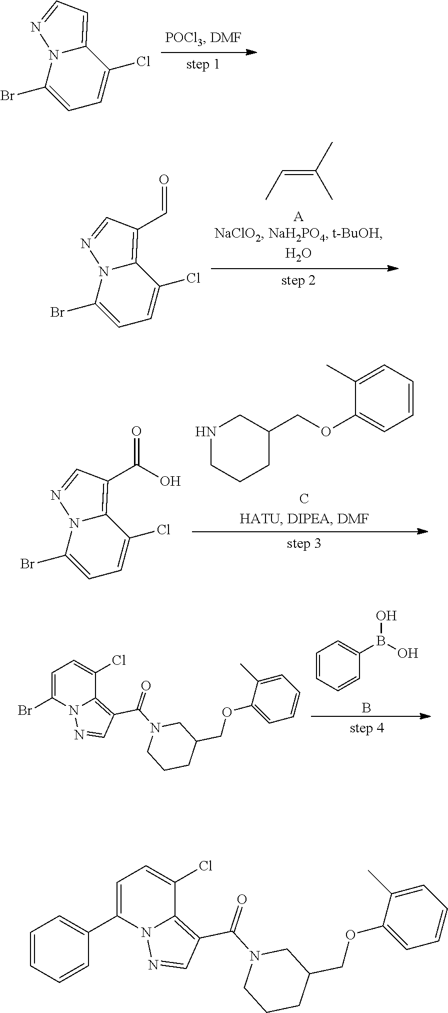

Step 1: 7-bromo-4-chloropyrazolo[1,5-a]pyridine-3-carbaldehyde: To a solution of 7-bromo-4-chloropyrazolo[1,5-a]pyridine (300 mg, 1.29 mmol, 1.00 equiv) was added POCl₃ (596 mg, 3.88 mmol, 3.00 equiv) at 0° C. The resulting mixture was stirred for 2 hours at room temperature under nitrogen atmosphere. The mixture was basified to pH 10 with 1M NaOH. The resulting mixture was extracted with CH₂Cl₂ (3×30 mL). The combined organic layers were washed with water (2×30 mL), dried over anhydrous Na₂SO₄. After filtration, the filtrate was concentrated under reduced pressure. This provided 7-bromo-4-chloropyrazolo[1,5-a]pyridine-3-carbaldehyde (150 mg, 44.6%) as a white solid. MS m/z: 260 [M+H]⁺.

Step 2: 7-bromo-4-chloropyrazolo[1,5-a]pyridine-3-carboxylic acid: To a stirred solution of 7-bromo-4-chloropyrazolo[1,5-a]pyridine-3-carbaldehyde (150 mg, 0.578 mmol, 1.00 equiv) in H₂O (1.00 mL) was added NaH₂PO₄ (416 mg, 3.46 mmol, 6.00 equiv) at 0° C. under air atmosphere. After 5 minutes, t-BuOH (5.00 mL), 2,3-dimethylbut-2-ene (122 mg, 1.45 mmol, 2.50 equiv) and NaClO₂ (78.4 mg, 0.867 mmol, 1.50 equiv) were added. After 16 hours, reaction mixture was diluted with ethyl acetate, washed with water and brine, then dried over magnesium sulfate, filtered, and concentrated to give 7-bromo-4-chloropyrazolo[1,5-a]pyridine-3-carboxylic acid (100 mg, 62.8%) as a white solid which was used without further purification. MS m/z: 277 [M+H]⁺.

Step 3: (7-bromo-4-chloropyrazolo[1,5-a]pyridin-3-yl)(3-((o-tolyloxy)methyl)piperidin-1-yl)methanone: To a stirred mixture of 7-bromo-4-chloropyrazolo[1,5-a]pyridine-3-carboxylic acid (100 mg, 0.363 mmol, 1.00 equiv) and 3-((o-tolyloxy)methyl)piperidine (112 mg, 0.544 mmol, 1.50 equiv) in DMF (3.00 mL) were added HATU (207 mg, 0.544 mmol, 1.50 equiv) and DIPEA (140 mg, 1.09 mmol, 3.00 equiv), The resulting mixture was stirred for 1 h at room temperature under argon atmosphere. The resulting mixture was diluted with water (10 mL) and extracted with EtOAc (3×20 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, ACN in water, 10% to 50% gradient in 10 min; detector, UV 254 nm. This provided 1-{7-bromo-4-chloropyrazolo[1,5-a]pyridine-3-carbonyl}-3-(2-methylphenoxymethyl)piperidine (100 mg, 59.5%) as a white solid. MS m/z: 464 [M+H]⁺.

Step 4: (4-chloro-7-phenylpyrazolo[1,5-a]pyridin-3-yl)(3-((o-tolyloxy)methyl)piperidin-1-yl)-methanone: To a solution of 1-{7-bromo-4-chloropyrazolo[1,5-a]pyridine-3-carbonyl}-3-(2-methylphenoxymethyl)piperidine (100 mg, 0.216 mmol, 1.00 equiv) and phenylboronic acid (39.5 mg, 0.324 mmol, 1.50 equiv) in dioxane (2.00 mL) and H₂O (0.50 mL) were added K₂CO₃ (59.7 mg, 0.430 mmol, 2.00 equiv) and Pd(dppf)Cl₂ (15.8 mg, 0.02 mmol, 0.100 equiv). After stirring for 2 hours at 80° C. under a nitrogen atmosphere, the resulting mixture was concentrated under reduced pressure. The residue was purified by reverse flash chromatography with the following conditions: column, C18 silica gel; mobile phase, ACN in water, 10% to 50% gradient in 10 min; detector, UV 254 nm. This provided 1-{4-chloro-7-phenylpyrazolo[1,5-a]pyridine-3-carbonyl}-3-(2-methylphenoxymethyl)piperidine (40 mg, 40.1%) as a white solid. ¹H NMR (300 MHZ, DMSO-d₆): δ 8.21-8.13 (m, 1H), 7.94-7.88 (m, 1H), 7.64-7.43 (m, 4H), 7.11-6.69 (m, 5H), 4.43-4.34 (m, 1H), 4.00-3.84 (m, 2H), 3.68-3.55 (m, 1H), 2.97 (s, 2H), 2.32-2.22 (s, 1H), 2.10-2.00 (m, 1H), 1.94-1.75 (m, 2H), 1.63-1.39 (m, 4H). MS m/z: 459.9 [M+H]⁺.

### 1-(2,2-Difluoroethyl)-6-(3-(1-((2-(trifluoromethyl)pyridin-3-yl)oxy)ethyl)piperidin-1-yl)-1H-pyrazolo[3,4-b]pyrazine (26)

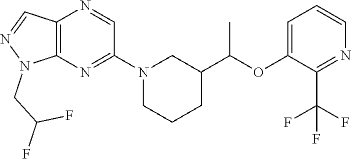 [SMILES: CC(Oc1cccnc1C(F)(F)F)C1CCCN(c2cnc3cnn(CC(F)F)c3n2)C1]

Step 1: tert-butyl 3-(1-{[2-(trifluoromethyl)pyridin-3-yl]oxy}ethyl)piperidine-1-carboxylate: Followed General Procedure A using 2-(trifluoromethyl)pyridin-3-ol (297 mg, 1.82 mmol, 1.00 equiv) and tert-butyl 3-(1-hydroxyethyl)piperidine-1-carboxylate (501 mg, 2.18 mmol. 1.2 equiv) to afford tert-butyl 3-(1-((2-(trifluoromethyl)pyridin-3-yl)oxy)ethyl)piperidine-1-carboxylate (30 mg, 4.4%) as a colorless oil. MS m/z: 375 [M+H]⁺.

Step 2: 3-(1-(piperidin-3-yl)ethoxy)-2-(trifluoromethyl)pyridine hydrochloride: Followed General Procedure B using tert-butyl 3-(1-((2-(trifluoromethyl)pyridin-3-yl)oxy)ethyl)piperidine-1-carboxylate (50 mg, 0.134 mmol, 1.00 equiv) to afford 3-(1-(piperidin-3-yl)ethoxy)-2-(trifluoromethyl)pyridine hydrochloride (50 mg). MS m/z: 275 [M+H]⁺.

Step 3 1-(2,2-difluoroethyl)-6-(3-(1-((2-(trifluoromethyl)pyridin-3-yl)oxy)ethyl)piperidin-1-yl)-1H-pyrazolo[3,4-b]pyrazine: Followed General Procedure C using 3-(1-(piperidin-3-yl)ethoxy)-2-(trifluoromethyl)pyridine hydrochloride (50 mg, 0.161 mmol, 1.00 equiv) and 6-chloro-1-(2,2-difluoroethyl)-1H-pyrazolo[3,4-b]pyrazine (38.7 mg, 0.177 mmol, 1.1 equiv). The crude product was purified by reverse flash chromatography with the following conditions: column, C18 silica gel; mobile phase, ACN in water, 10% to 50% gradient in 30 min; detector, UV 254/220 nm to afford 1-(2,2-difluoroethyl)-6-(3-(1-((2-(trifluoromethyl)pyridin-3-yl)oxy)ethyl)piperidin-1-yl)-1H-pyrazolo[3,4-b]pyrazine (13.9 mg, 18.6%) as an off-white solid. ¹H NMR (400 MHZ, CD₃OD): δ 8.32 (s, 1H), 8.19-8.16 (m, 2H), 7.97 (s, 1H), 7.77-7.74 (m, 1H), 7.62-7.56 (m, 1H), 6.40-6.09 (m, 1H), 4.78-4.72 (m, 1H), 4.69-4.59 (m, 3H), 4.54-4.48 (m, 1H), 3.16-3.05 (m, 2H), 2.11-1.87 (m, 3H), 1.73-1.60 (m, 2H), 1.41-1.38 (m, 3H). MS m/z: 457.20 [M+H]⁺.

### 5-(Phenoxymethyl)-1-(quinoxalin-2-yl)piperidin-2-one (27)

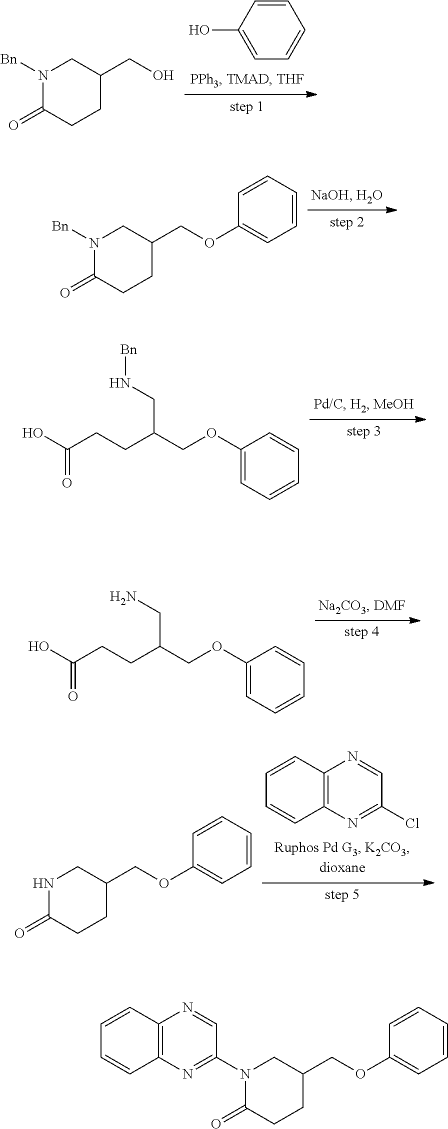

Step 1: 1-benzyl-5-(phenoxymethyl)piperidin-2-one: Followed General Procedure A using 1-benzyl-5-(hydroxymethyl)piperidin-2-one (190 mg, 0.787 mmol, 1.00 equiv), phenol (148 mg, 1.57 mmol, 1.5 equiv) to afford 1-benzyl-5-(phenoxymethyl)piperidin-2-one (140 mg, 56.0%) as a white solid. MS m/z: 296 [M+H]⁺.

Step 2: 5-(benzylamino)-4-(phenoxymethyl)pentanoic acid: To a solution of methyl 1-benzyl-5-(phenoxymethyl)piperidin-2-one (200 mg, 0.493 mmol, 1.00 equiv) in MeOH (2.00 mL) was added NaOH (78.9 mg, 1.97 mmol, 4.00 equiv) in water (1.00 mL). The mixture was stirred at 100° C. for 1 h. The mixture was concentrated by 3M HCl aq, and the resulting mixture was purified by reverse flash chromatography with the following conditions: column, C18 silica gel; mobile phase, MeCN in water, 5% to 95% gradient in 15 min; detector, UV 254 nm to afford 5-(benzylamino)-4-(phenoxymethyl)pentanoic acid (150 mg, 77.7%) as a white solid. MS m/z: 297 [M+H]⁺.

Step 3: 5-amino-4-(phenoxymethyl)pentanoic acid: To a solution of 5-(benzylamino)-4-(phenoxymethyl)pentanoic acid (100 mg, 0.333 mmol, 1.00 equiv) in MeOH (5.00 mL) was added Pd/C (16.6 mg) with water. The resulted mixture was hydrogenated overnight at room temperature. Desired product could be detected by LCMS. The reaction system was filtrated through celite and the filtrate was concentrated. The crude product 5-amino-4-(phenoxymethyl)pentanoic acid (103 mg, crude) was used directly for next step. MS m/z: 214 [M+H]⁺.

Step 4: 5-(phenoxymethyl)piperidin-2-one: To a stirred solution of 5-amino-4-(phenoxymethyl)pentanoic acid (200 mg, 1.83 mmol, 1.00 equiv) in DMF (5.00 mL) was added sat. Na₂CO₃ (1.6 mL) at 0° C. The mixture was stirred for 2 h at 100° C. The resulting mixture was diluted with DCM (50 mL), washed with water (2×50 mL) and brine (1×50 mL), and was dried over anhydrous sodium sulfate. After filtration, the filtrate was concentrated under reduced pressure. The crude product was purified by reverse phase flash with the following conditions (column, C18 gel; mobile phase, B phase: MeCN, A phase: water; 5% to 95% B gradient in 15 min; detector: UV 220/200 nm) to afford 5-(phenoxymethyl)piperidin-2-one (640 mg, 86.0%) as a colorless syrup. MS m/z: 206 [M+H]⁺.

Step 5: 5-(phenoxymethyl)-1-(quinoxalin-2-yl)piperidin-2-one: To the solution of 5-(phenoxymethyl)piperidin-2-one (50.0 mg, 0.143 mmol, 1.00 equiv) and 2-chloroquinoxaline (44.0 mg, 0.215 mmol, 1.50 equiv) in dioxane (3.00 mL) were added RuPhos Pd G3 (5.9 mg, 0.007 mmol, 0.05 equiv) and K₂CO₃ (54.9 mg, 0.286 mmol, 2.00 equiv) under N₂ atmosphere. The result mixture was heated to 60° C. and stirred overnight. Desired product could be detected by LCMS. The reaction mixture was diluted by EtOAc (20 mL), washed by water (2×20 mL) and brine (1×10 mL), dried over anhydrous sodium sulfate. After filtration, the filtrate was concentrated. The residue was purified by silica gel column chromatography, eluted with EtOAc/PE=1/10 to afford 5-(phenoxymethyl)-1-(quinoxalin-2-yl)piperidin-2-one (15 mg, 86.0%) as a white solid. ¹H NMR (400 MHZ, DMSO-d₆) δ 9.30 (s, 1H), 8.15-8.12 (m, 1H), 8.04-7.91 (m, 1H), 7.89-7.81 (m, 2H), 7.38-7.33 (m, 2H), 7.06-6.98 (m, 3H), 4.34-4.28 (m, 1H), 4.16-4.13 (m, 2H), 4.00-3.93 (m, 1H), 2.80-2.61 (m, 3H), 2.19-2.13 (m, 1H), 1.91-1.84 (m, 1H). MS m/z: 334.0 [M+H]⁺.

### 3-(Phenoxymethyl)-1-(1-phenyl-1H-1,2,3-triazol-4-yl)piperidine (28)

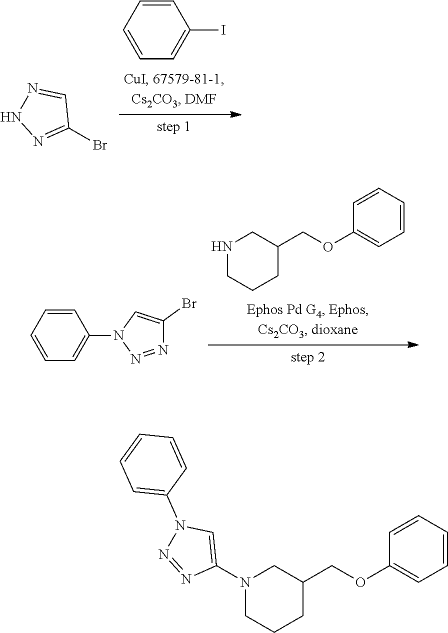 [SMILES: Ic1ccccc1 ; Brc1cn[nH]n1 ; c1ccc(OCC2CCCNC2)cc1 ; Brc1cn(-c2ccccc2)nn1 ; c1ccc(OCC2CCCN(c3cn(-c4ccccc4)nn3)C2)cc1]

Step 1: 4-bromo-1-phenyl-1H-1,2,3-triazole: To the solution of 4-bromo-2H-1,2,3-triazole (400 mg, 2.70 mmol, 1.00 equiv) and iodophenyl (1654 mg, 8.11 mmol, 3 equiv) in DMF (5 mL) were added CuI (51.5 mg, 0.270 mmol, 0.1 equiv), (1S,2S)-1-N,2-N-dimethylcyclohexane-1,2-diamine (38.5 mg, 0.270 mmol, 0.10 equiv) and Cs₂CO₃ (2642 mg. 8.11 mmol, 3 equiv) under N₂ atmosphere. The result mixture was heated to 100° C. and stirred overnight. Desired product could be detected by LCMS. The reaction mixture was diluted by EtOAc (30 mL), washed by water (2×30 mL) and brine (1×30 mL), dried over anhydrous sodium sulfate. After filtration, the filtrate was concentrated. The residue was purified by silica gel column chromatography, eluted with EtOAc/PE=1/1 to afford 4-bromo-1-phenyl-1H-1,2,3-triazole (100 mg, 16.5%). MS m/z: 224 [M+H]⁺.

Step 2: 3-(phenoxymethyl)-1-(1-phenyl-1H-1,2,3-triazol-4-yl)piperidine: To a solution of 4-bromo-1-phenyl-1H-1,2,3-triazole (60 mg, 0.268 mmol, 1.00 equiv) and 3-(phenoxymethyl)piperidine hydrochloride (61.0 mg, 0.268 mmol, 1 equiv) in dioxane (2 mL) were added Ephos Pd G4 (24.6 mg, 0.027 mmol, 0.1 equiv), Ephos (14.3 mg, 0.027 mmol, 0.1 equiv) and Cs₂CO₃ (262 mg, 0.804 mmol, 3 equiv) under N₂ atmosphere. The result mixture was heated to 90° C. and stirred overnight. Desired product could be detected by LCMS. The reaction mixture was diluted by EtOAc (20 mL), washed by water (2×20 mL) and brine (1×10 mL), dried over anhydrous sodium sulfate. After filtration, the filtrate was concentrated. The residue was purified by silica gel column chromatography, eluted with EtOAc/PE=1/1 to afford impure product. The impure product was further purified by reverse flash chromatography with the following conditions: column, C18 silica gel; mobile phase, MeCN in water, 35% to 75% gradient in 15 min; detector, UV 254 nm. This provided 3-(phenoxymethyl)-1-(1-phenyl-1H-1,2,3-triazol-4-yl)piperidine (10 mg, 11.2%) as a white solid. ¹H NMR (400 MHZ, DMSO-d₆) δ 8.20 (s, 1H), 8.04-7.76 (m, 2H), 7.57 (t, J=7.8 Hz, 2H), 7.44 (t, J=7.4 Hz, 1H), 7.39-7.21 (m, 2H), 7.09-6.76 (m, 3H), 4.11-3.88 (m, 2H), 3.84 (dd, J=11.7, 3.8 Hz, 1H), 3.70-3.54 (m, 1H), 2.76 (td, J=11.6, 3.1 Hz, 1H), 2.71-2.62 (m, 2H), 2.24-2.05 (m, 1H), 1.94-1.82 (m, 1H), 1.82-1.74 (m, 1H), 1.70-1.58 (m, 1H), 1.40-1.17 (m, 1H). MS m/z: 335.0 [M+H]⁺.

### (1H-Indol-6-yl)(3-((phenylsulfonyl)methyl)piperidin-1-yl)methanone (29)

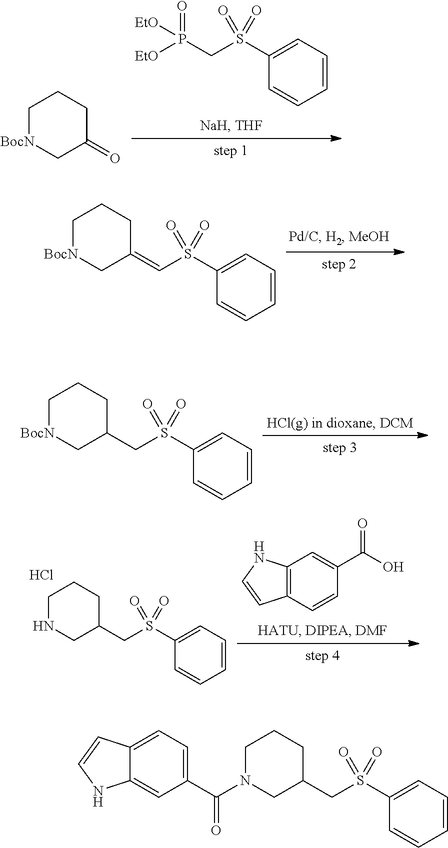

Step 1: tert-butyl (E)-3-((phenylsulfonyl)methylene)piperidine-1-carboxylate: To a stirred mixture of diethyl((phenylsulfonyl)methyl)phosphonate (880 mg, 3.01 mmol, 1.5 equiv) in THF (8 mL) was added NaH (60% w/z oil, 120 mg, 3.01 mmol, 1.50 equiv) dropwise at 0° C. under N₂ atmosphere. The resulting mixture was allowed to warm to room temperature and was stirred for 30 min under N₂ atmosphere. The reaction system was then cooled to 0° C. To the stirred solution was added tert-butyl 3-oxopiperidine-1-carboxylate (399 mg, 2 mmol, 1.00 equiv) in THF (4.00 mL) dropwise at 0° C. under N₂ atmosphere. The resulting mixture was allowed to warm to room temperature and was stirred for 3 h at room temperature under N₂ atmosphere. Desired product could be detected by LCMS. The reaction was quenched with saturated NH₄HCO₃ aq. at 0° C. The resulting mixture was extracted with EtOAc (3×20 mL). The combined organic layers were washed with water (2×20 mL) and brine (1×20 mL), dried over anhydrous Na₂SO₄. After filtration, the filtrate was concentrated under reduced pressure. The residue was purified by silica gel column chromatography, eluted with EtOAc/PE (1/3) to afford tert-butyl (E)-3-((phenylsulfonyl)methylene)piperidine-1-carboxylate (200 mg, 29.5%) as a colorless oil. MS m/z: 338 [M+H]⁺.

Step 2: tert-butyl 3-((phenylsulfonyl)methyl)piperidine-1-carboxylate: To a stirred solution of tert-butyl (3E)-3-[(benzenesulfonyl)methylidene]piperidine-1-carboxylate (200 mg, 0.593 mmol, 1.00 equiv) in MeOH (10 mL) was added Pd/C (20 mg, 10% Pd on carbon, wetted with water). The resulting mixture was stirred for overnight at room temperature under hydrogen atmosphere. The resulting mixture was filtered, the filter cake was washed with MeOH (3×10 mL). The filtrate was concentrated under reduced pressure. This provided tert-butyl 3-[(benzenesulfonyl)methyl]piperidine-1-carboxylate (165 mg, 82.01%) as a yellow oil. MS m/z: 340 [M+H]⁺.

Step 3: 3-((phenylsulfonyl)methyl)piperidine: To a stirred solution of tert-butyl tert-butyl 3-[(benzenesulfonyl)methyl]piperidine-1-carboxylate (120 mg, 0.354 mmol, 1.00 equiv) in DCM (3 mL) was added HCl (g) in dioxane (1.5 mL) dropwise at 0° C. The resulting mixture was stirred for 2 hours at room temperature. After removing the solvent, the crude product 3-[(benzenesulfonyl)methyl]piperidine (130 mg) was used for next step without further purification. MS m/z: 240 [M+H]⁺.

Step 4: (1H-indol-6-yl)(3-((phenylsulfonyl)methyl)piperidin-1-yl)methanone: A mixture of 1H-indole-6-carboxylic acid (91.8 mg, 0.570 mmol, 1.00 equiv), 3-[(benzenesulfonyl)methyl]piperidine (130 mg, 0.546 mmol, 1.1 equiv) and HATU (325 mg, 0.855 mmol, 1.5 equiv) were added to DMF (2.00 mL) followed by DIPEA (96.2 mg, 0.744 mmol, 1.5 equiv) at room temperature. The mixture was stirred at room temperature for 16 h. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford (1H-indol-6-yl)(3-((phenylsulfonyl)methyl)piperidin-1-yl)methanone (19 mg, 9.96%) as a white solid. ¹H NMR (300 MHZ, DMSO-d₆) δ 11.28 (s, 1H), 7.98-7.33 (m, 8H), 6.97 (d, J=8.1 Hz, 1H), 6.50 (t, J=2.4 Hz, 1H), 4.22 (s, 2H), 3.29 (s, 2H), 2.97-2.68 (m, 2H), 1.88 (t, J=12.8 Hz, 2H), 1.60 (d, J=11.3 Hz, 1H), 1.44-1.17 (m, 2H). MS m/z: 393.1 [M+H]⁺.

### 2-(1-Ethyl-6-(3-((o-tolyloxy)methyl)piperidin-1-yl)-1H-imidazo[4,5-b]pyrazin-2-yl)-1,3,4-thiadiazole (30)

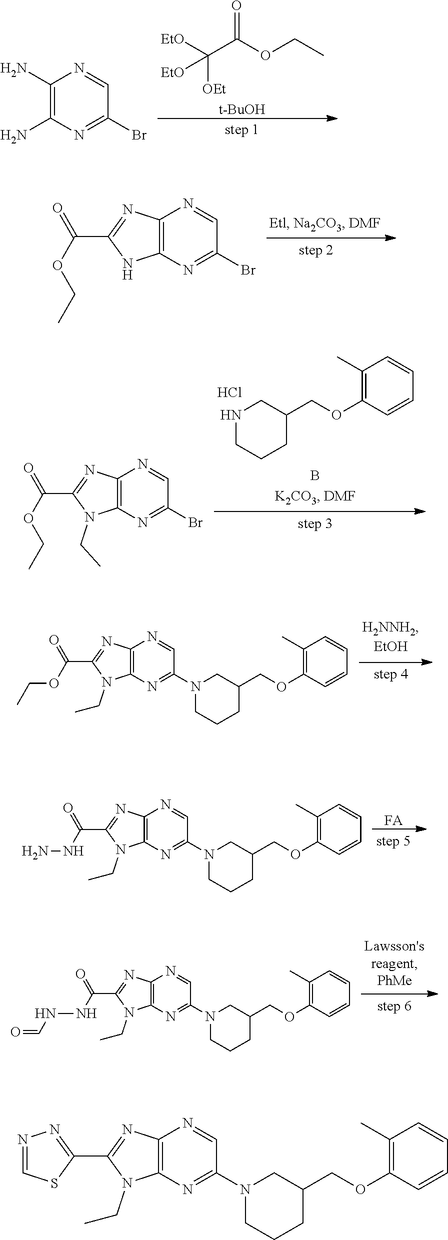

Step 1: ethyl 6-bromo-1H-imidazo[4,5-b]pyrazine-2-carboxylate: A solution of 5-bromopyrazine-2,3-diamine (1 g, 5.29 mmol, 1.00 equiv) and ethyl 2,2,2-diethoxyacetate (3.5 g, 15.8 mmol, 3.0 equiv) in 2-methylpropan-2-ol (10 mL) was stirred for 3 days at 100° C. The mixture was allowed to cool down to room temperature and was concentrated under vacuum. The residue was purified by reverse flash chromatography with the following conditions: column, C18 silica gel; mobile phase, MeCN in water, 5% to 95% gradient in 30 min; detector, UV 254 nm. This provided ethyl 6-bromo-1H-imidazo[4,5-b]pyrazine-2-carboxylate (700 mg, 48.8%) as a yellow solid. MS m/z: 271 [M+H]⁺.

Step 2: ethyl 6-bromo-1-ethyl-1H-imidazo[4,5-b]pyrazine-2-carboxylate: A solution of ethyl 6-bromo-1H-imidazo[4,5-b]pyrazine-2-carboxylate (700 mg, 2.58 mmol, 1 equiv) and ethyl iodide (483 mg, 3.10 mmol, 1.2 equiv) in DMF (5 mL) was stirred for overnight at room temperature. The resulting mixture was diluted with EtOAc (40 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (2:1) to afford ethyl 6-bromo-1-ethyl-1H-imidazo[4,5-b]pyrazine-2-carboxylate (400 mg, 51.8%) as a yellow solid. MS m/z: 299 [M+H]⁺.

Step 3: ethyl 1-ethyl-6-(3-((o-tolyloxy)methyl)piperidin-1-yl)-1H-imidazo[4,5-b]pyrazine-2-carboxylate: A solution of ethyl 6-bromo-1-ethyl-1H-imidazo[4,5-b]pyrazine-2-carboxylate (400 mg, 1.34 mmol, 1 equiv), 3-(2-methylphenoxymethyl)piperidine (302 mg, 1.471 mmol, 1.1 equiv) and Na₂CO₃ (283 mg, 2.674 mmol, 2 equiv) in DMF (5 mL) was stirred for 3 h at 100° C. The resulting mixture was diluted with EtOAc (30 mL). The organic layer was washed with water (3×20 mL), dried over anhydrous Na₂SO₄. After filtration, the filtrate was concentrated under reduced pressure. The residue was purified by reverse flash chromatography with the following conditions: column, silica gel; mobile phase, MeCN in water, 10% to 95% gradient in 30 min; detector, UV 254 nm. This provided ethyl 1-ethyl-6-(3-((o-tolyloxy)methyl)piperidin-1-yl)-1H-imidazo[4,5-b]pyrazine-2-carboxylate (290 mg, 51.21%) as a light yellow solid. MS m/z: 424 [M+H]⁺.

Step 4: 1-ethyl-6-(3-((o-tolyloxy)methyl)piperidin-1-yl)-1H-imidazo[4,5-b]pyrazine-2-carbohydrazide: A solution of ethyl 1-ethyl-6-(3-((o-tolyloxy)methyl)piperidin-1-yl)-1H-imidazo[4,5-b]pyrazine-2-carboxylate (290 mg, 0.685 mmol, 1 equiv) in hydrazine (4 mL) was stirred for 2 h at 80° C. The resulting mixture was concentrated under vacuum. The resulting crude product was used in the next step directly without further purification. MS m/z: 410 [M+H]⁺.

Step 5: 1-ethyl-N′-formyl-6-(3-((o-tolyloxy)methyl)piperidin-1-yl)-1H-imidazo[4,5-b]pyrazine-2-carbohydrazide: A solution of 1-ethyl-6-(3-((o-tolyloxy)methyl)piperidin-1-yl)-1H-imidazo[4,5-b]pyrazine-2-carbohydrazide (290 mg, 0.708 mmol, 1 equiv) in HCOOH (5 mL) was stirred for 2 h at 80° C. The residue was purified by reverse flash chromatography with the following conditions: column, silica gel; mobile phase, MeCN in water, 10% to 100% gradient in 30 min; detector, UV 254 nm. This provided 1-ethyl-N′-formyl-6-(3-((o-tolyloxy)methyl)piperidin-1-yl)-1H-imidazo[4,5-b]pyrazine-2-carbohydrazide (200 mg, 64.5%) as a yellow solid. MS m/z: 438 [M+H]⁺.

Step 6: 2-(1-ethyl-6-(3-((o-tolyloxy)methyl)piperidin-1-yl)-1H-imidazo[4,5-b]pyrazin-2-yl)-1,3,4-thiadiazole: A solution of 1-ethyl-N′-formyl-6-(3-((o-tolyloxy)methyl)piperidin-1-yl)-1H-imidazo[4,5-b]pyrazine-2-carbohydrazide (30 mg, 0.069 mmol, 1.00 equiv) and Lawesson reagent (16.6 mg, 0.041 mmol, 0.60 equiv) in PhCH₃ (2 mL) was stirred for 3 h at 100° C. The resulting mixture was concentrated under vacuum. The residue was purified by silica gel column chromatography, eluted with PE/EA (2:1) to afford 2-(1-ethyl-6-(3-((o-tolyloxy)methyl)piperidin-1-yl)-1H-imidazo[4,5-b]pyrazin-2-yl)-1,3,4-thiadiazole as a yellow solid. The residue was further purified by reverse flash chromatography with the following conditions: column, silica gel; mobile phase, MeCN in water, 10% to 90% gradient in 30 min; detector, UV 254 nm. This provided 2-(1-ethyl-6-(3-((o-tolyloxy)methyl)piperidin-1-yl)-1H-imidazo[4,5-b]pyrazin-2-yl)-1,3,4-thiadiazole (12.2 mg, 40.0%) as a yellow solid. ¹H NMR (400 MHZ, CDCl₃) δ 9.18 (s, 1H), 8.27 (s, 1H), 7.16 (t, J=7.7 Hz, 2H), 6.99-6.74 (m, 2H), 4.90 (q. J=7.1 Hz, 2H), 4.72-4.62 (m, 1H), 4.33 (d, J=12.9 Hz, 1H), 4.00 (dd, J=9.2, 4.8 Hz, 1H), 3.93-3.83 (m, 1H), 3.23-3.12 (m, 1H), 3.10-2.98 (m, 1H), 2.31 (s, 3H), 2.30-2.19 (m, 1H), 2.05-1.96 (m, 1H), 1.95-1.87 (m, 1H), 1.86-1.67 (m, 1H), 1.60-1.46 (m, 4H). MS m/z: 435.9 [M+H]⁺

### 1-(2,2-Difluoroethyl)-6-(2-methyl-5-(((2-(trifluoromethyl)pyridin-3-yl)oxy)methyl)piperidin-1-yl)-1H-pyrazolo[3,4-b]pyrazine (31)

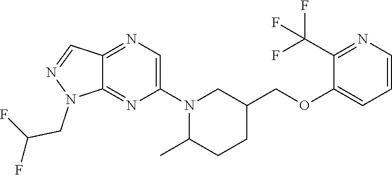 [SMILES: CC1CCC(COc2cccnc2C(F)(F)F)CN1c1cnc2cnn(CC(F)F)c2n1]

Step 1: tert-butyl 2-methyl-5-(((2-(trifluoromethyl)pyridin-3-yl)oxy)methyl)piperidine-1-carboxylate: Followed General Procedure A using tert-butyl 5-(hydroxymethyl)-2-methylpiperidine-1-carboxylate (100 mg, 0.436 mmol, 1.00 equiv) and 2-(trifluoromethyl)pyridin-3-ol (64 mg, 0.392 mmol, 0.9 equiv) to afford tert-butyl 2-methyl-5-(((2-(trifluoromethyl)pyridin-3-yl)oxy)methyl)piperidine-1-carboxylate (70 mg, 42.8%) as a colorless oil. MS m/z: 375 [M+H]⁺.

Step 2: 3-((6-methylpiperidin-3-yl)methoxy)-2-(trifluoromethyl)pyridine hydrochloride: Followed General Procedure B using tert-butyl 2-methyl-5-(((2-(trifluoromethyl)pyridin-3-yl)oxy)methyl)piperidine-1-carboxylate (70 mg, 0.187 mmol, 1.00 equiv) to afford 3-((6-methylpiperidin-3-yl)methoxy)-2-(trifluoromethyl)pyridine hydrochloride (50 mg). MS m/z: 275 [M+H]⁺.

Step 3: 1-(2,2-difluoroethyl)-6-(2-methyl-5-(((2-(trifluoromethyl)pyridin-3-yl)oxy)methyl)piperidin-1-yl)-1H-pyrazolo[3,4-b]pyrazine: Followed General Procedure C using 3-((6-methylpiperidin-3-yl)methoxy)-2-(trifluoromethyl)pyridine hydrochloride (50 mg, 0.161 mmol, 1.00 equiv) and 6-chloro-1-(2,2-difluoroethyl)-1H-pyrazolo[3,4-b]pyrazine (38.7 mg, 0.177 mmol, 1.1 equiv). The crude product was purified by reverse flash chromatography with the following conditions: column, C18 silica gel; mobile phase, ACN in water, 10% to 50% gradient in 30 min; detector, UV 254/220 nm to afford 1-(2,2-difluoroethyl)-6-(2-methyl-5-(((2-(trifluoromethyl)pyridin-3-yl)oxy)methyl)piperidin-1-yl)-1H-pyrazolo[3,4-b]pyrazine (17.3 mg, 22.9%) as a white solid. ¹H NMR (400 MHZ. CD₃OD): δ 8.32 (s, 1H), 8.23-8.10 (m, 1H), 7.99 (s, 1H), 7.77-7.72 (m, 1H), 7.66-7.61 (m, 1H), 6.40-6.09 (m, 1H), 4.94-4.88 (m, 1H), 4.81-4.76 (m, 1H), 4.72-4.62 (m, 2H), 4.29-4.24 (m, 1H), 4.12-4.06 (m, 1H), 3.07-2.97 (m, 1H), 2.23-2.13 (m, 1H), 2.00-1.90 (m, 1H), 1.88-1.74 (m, 3H), 1.35-1.29 (m, 3H). MS m/z: 457.2 [M+H]⁺.

### 5-Methyl-6-phenyl-3-(3-((o-tolyloxy)methyl)piperidin-1-yl)-5H-pyrrolo[2,3-b]pyrazine 32)

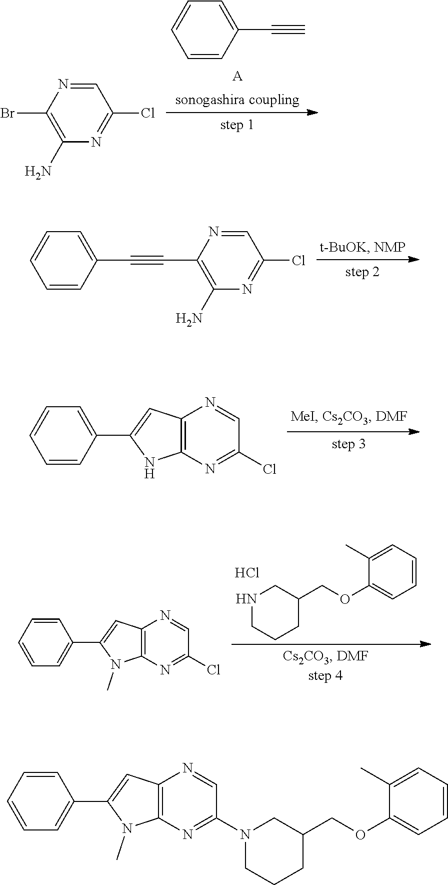

Step 1: 6-chloro-3-(2-phenylethynyl)pyrazin-2-amine: To a stirred mixture of 3-bromo-6-chloropyrazin-2-amine (1 g, 4.79 mmol, 1.00 equiv), ethynylbenzene (0.74 g, 7.19 mmol, 1.50 equiv), CuI (0.09 g, 0.480 mmol, 0.1 equiv) and PPh₃ (2.52 g, 9.59 mmol, 2 equiv) in DMF (10 mL) were added Pd(PPh₃)₂Cl₂ (0.34 g, 0.480 mmol, 0.1 equiv) and TEA (1.46 g, 14.3 mmol, 3 equiv) at room temperature. The resulting mixture was stirred for 16 hours at 80° C. under N₂ atmosphere. The resulting mixture was diluted with EtOAc (40 mL). The resulting mixture was washed with water (2×40 mL). The combined organic layers were washed with brine (40 mL), dried over anhydrous sodium sulfate. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 6-chloro-3-(2-phenylethynyl)pyrazin-2-amine (870 mg, 79.0%) as a yellow solid. MS m/z: 230[M+H]⁺.

Step 2: 3-chloro-6-phenyl-5H-pyrrolo[2,3-b]pyrazine: A solution of 6-chloro-3-(2-phenylethynyl)pyrazin-2-amine (200 mg, 0.871 mmol, 1.00 equiv) and t-BuOK (200 mg, 1.78 mmol, 2.05 equiv) in NMP (3 mL) was stirred for 2 hours at 80° C. The residue was purified by reverse flash chromatography with the following conditions: column, C18 silica gel; mobile phase, MeOH in water, 0% to 100% gradient in 30 min; detector, UV 254 nm. This provided 3-chloro-6-phenyl-5H-pyrrolo[2,3-b]pyrazine (175 mg, 87.5%) as a yellow solid. MS m/z: 230 [M+H]⁺.

Step 3: 3-chloro-5-methyl-6-phenylpyrrolo[2,3-b]pyrazine: To a stirred solution of 3-chloro-6-phenyl-5H-pyrrolo[2,3-b]pyrazine (170 mg, 0.740 mmol, 1.00 equiv) and Cs₂CO₃ (723 mg, 2.22 mmol, 3 equiv) in DMF (2 mL) was added MeI (126 mg, 0.888 mmol, 1.2 equiv) at room temperature. The resulting mixture was stirred for 3 hours at room temperature. The residue was purified by reverse flash chromatography with the following conditions: column, C18 silica gel; mobile phase, MeCN in water, 0% to 100% gradient in 30 min; detector, UV 254 nm. This provided 3-chloro-5-methyl-6-phenylpyrrolo[2,3-b]pyrazine (150 mg, 83.2%) as a yellow solid. MS m/z: 244 [M+H]⁺.

Step 4: 5-methyl-6-phenyl-3-(3-((o-tolyloxy)methyl)piperidin-1-yl)-5H-pyrrolo[2,3-b]pyrazine: To a stirred solution of 3-chloro-5-methyl-6-phenylpyrrolo[2,3-b]pyrazine (50 mg, 0.205 mmol, 1.00 equiv) and 3-((o-tolyloxy)methyl)piperidine hydrochloride (54.7 mg, 0.267 mmol, 1.3 equiv) in dioxane (1 mL) were added 1612891-29-8 (17.2 mg, 0.021 mmol, 0.1 equiv) and Cs₂CO₃ (200 mg, 0.615 mmol, 3 equiv) at room temperature under N₂ atmosphere. The resulting mixture was stirred for 2 hours at 100° C. under N₂ atmosphere. Desired product could be detected by LCMS. The reaction mixture was diluted by EtOAc (20 mL), washed by water (2×20 mL) and brine (1×20 mL), dried over anhydrous sodium sulfate. After filtration, the filtrate was concentrated. The residue was purified by reverse flash chromatography with the following conditions: column, C18 silica gel; mobile phase, MeCN in water, 0% to 100% gradient in 30 min; detector, UV 254 nm. This provided 5-methyl-6-phenyl-3-(3-((o-tolyloxy)methyl) piperidin-1-yl)-5H-pyrrolo[2,3-b]pyrazine (14 mg, 16.47%) as a yellow solid. ¹H NMR (400 MHZ, DMSO-d₆) δ 8.13 (s, 1H). 7.62-7.53 (m, 2H), 7.51-7.42 (m, 2H), 7.41-7.32 (m, 1H), 7.09-7.05 (m, 2H), 6.94-6.82 (m, 1H), 6.78-6.74 (m, 1H), 6.52 (s, 1H), 4.51-4.46 (m, 1H), 4.20-4.17 (m, 1H), 3.94-3.90 (m, 1H), 3.85-3.81 (m, 1H), 3.62 (s, 3H), 3.05-2.94 (m, 1H), 2.88-2.82 (m, 1H), 2.17 (s. 3H), 2.05-2.00 (m, 1H), 1.92-1.80 (m, 1H), 1.75-1.70 (m, 1H), 1.57-1.48 (m, 1H), 1.45-1.28 (m, 1H). MS m/z: 413.0 [M+H]⁺.

### 5-Methyl-6-phenyl-3-(3-(((2-(trifluoromethyl)pyridin-3-yl)oxy)methyl)piperidin-1-yl)-5H-pyrrolo[2,3-b]pyrazine (33)

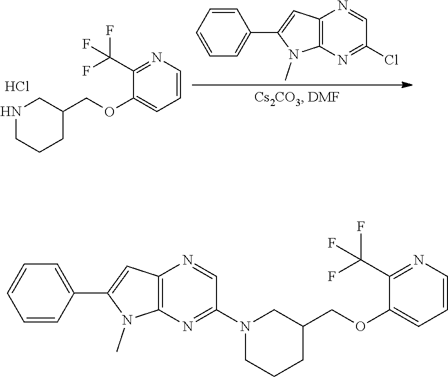

To a stirred solution of 3-(piperidin-3-ylmethoxy)-2-(trifluoromethyl)pyridine hydrochloride (94.9 mg, 0.320 mmol, 1.3 equiv) and 3-chloro-5-methyl-6-phenyl-5H-pyrrolo[2,3-b]pyrazine (60 mg, 0.246 mmol, 1.00 equiv) in dioxane (2 mL) were added 1612891-29-8 (20.7 mg, 0.025 mmol, 0.1 equiv) and Cs₂CO₃ (160 mg, 0.492 mmol, 2 equiv) at room temperature under N₂ atmosphere. The resulting mixture was stirred for 2 hours at 100° C. under N₂ atmosphere. Desired product could be detected by LCMS. The reaction mixture was diluted by EtOAc (20 mL), washed by water (2×20 mL) and brine (1×20 mL), dried over anhydrous sodium sulfate. After filtration, the filtrate was concentrated. The residue was purified by reverse flash chromatography with the following conditions: column, C18 silica gel; mobile phase, MeCN in water, 0% to 100% gradient in 30 min; detector, UV 254 nm. This provided 5-methyl-6-phenyl-3-(3-(((2-(trifluoromethyl)pyridin-3-yl)oxy)methyl)piperidin-1-yl)-5H-pyrrolo[2,3-b]pyrazine (17 mg, 14.19%) as a yellow solid. ¹H NMR (400 MHz, DMSO-d₆) δ 8.26 (dd, J=4.5, 1.2 Hz, 1H), 8.19 (s, 1H), 7.85-7.80 (m, 1H), 7.72-7.67 (m, 1H), 7.67-7.60 (m, 2H), 7.56-7.47 (m, 2H), 7.47-7.39 (m, 1H), 6.60 (s, 1H), 4.54 (d, J=11.1 Hz, 1H), 4.30-4.18 (m, 2H), 4.15-4.07 (m, 1H), 3.68 (s, 3H), 3.03 (t, J=11.0 Hz, 1H), 2.96-2.86 (m, 1H), 2.14 (s, 1H), 1.96-1.86 (m, 1H), 1.86-1.76 (m, 1H), 1.67-1.53 (m, 1H), 1.50-1.39 (m, 1H). MS m/z: 468.3 [M+H]⁺.

### 3-(Phenoxymethyl)-1-[3-(trifluoromethyl)-5H,6H,7H,8H-[1,2,4]triazolo[4,3-a]pyrazine-7-carbonyl]piperidine (34)

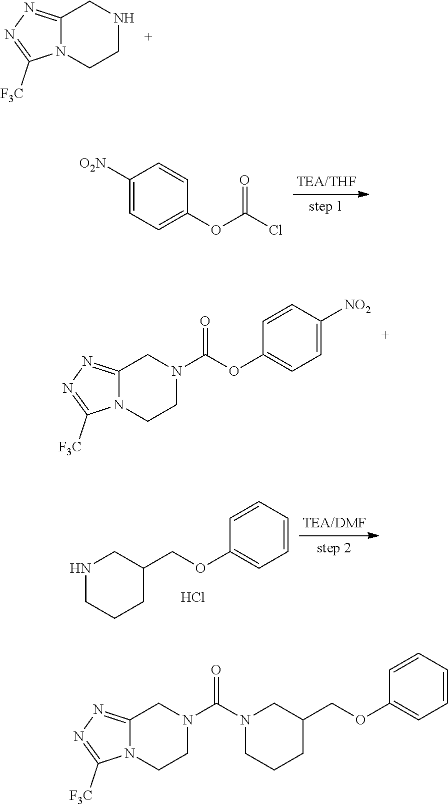

Step 1: 4-nitrophenyl 3-(trifluoromethyl)-5H,6H,7H,8H-[1,2,4]triazolo[4,3-a]pyrazine-7-carboxylate: A mixture of 3-(trifluoromethyl)-5H,6H,7H,8H-[1,2,4]triazolo[4,3-a]pyrazine hydrochloride (100 mg, 437 μmol, 1.0 equiv), 4-nitrophenyl carbonochloridate (97.0 mg, 1.1 equiv, 481 μmol), and TEA (122 μL, 2 eq., 875 μmol) in THF (2.00 mL) was stirred at rt for 16 h. The mixture was filtered. The filtrate was concentrated and purified by Combi-flash to give a white solid (85 mg, 54%) MS m/z: 358 [M+H]⁺.

Step 2: 3-(phenoxymethyl)-1-[3-(trifluoromethyl)-5H,6H,7H,8H-[1,2,4]triazolo[4,3-a]pyrazine-7-carbonyl]piperidine: A mixture of 4-nitrophenyl 3-(trifluoromethyl)-5H,6H,7H,8H-[1,2,4]triazolo[4,3-a]pyrazine-7-carboxylate (20.0 mg, 56.0 μmol), 3-(phenoxymethyl)piperidine hydrochloride (12.7 mg, 56.0 μmol), and TEA (9.36 UL, 1.2 eq., 67.2 μmol) in DMF (1.00 mL) was heated at 70° C. overnight. After the completion of the reaction determined by LC-MS. The mixture was added water (2 mL) and extracted with EtOAc (3 mL×2). The organic layer was concentrated and purified by Combi-Flash to afford 3-(phenoxymethyl)-1-[3-(trifluoromethyl)-5H,6H,7H,8H-[1,2,4]triazolo[4,3-a]pyrazine-7-carbonyl]piperidine as a light yellow oil (21 mg, 92%). ¹H NMR (500 MHZ, CDCl₃) δ 7.30-7.26 (m, 2H), 6.95 (tt, J=7.3, 1.1 Hz, 1H), 6.88-6.84 (m, 2H), 4.70 (d, J=1.4 Hz, 2H), 4.28-4.15 (m, 2H), 3.93-3.83 (m, 2H), 3.79 (dd, J=9.4, 8.1 Hz, 1H), 3.73-3.56 (m, 3H), 2.95 (td, J=11.8, 11.3, 3.0 Hz, 1H), 2.90-2.78 (m, 1H), 2.17-2.07 (m, 1H), 1.94 (dq, J=13.0, 4.1 Hz, 1H), 1.81 (dt, J=13.6, 3.7 Hz, 1H), 1.39 (dtd, J=13.1, 11.2, 4.0 Hz, 1H). MS m/z: 410 [M+H]⁺.

### 3-({1-[1-(2,2-Difluoroethyl)-1H-pyrazolo[3,4-b]pyrazin-6-yl]-3-fluoropiperidin-3-yl}methoxy)-2-(trifluoromethyl)pyridine (35)

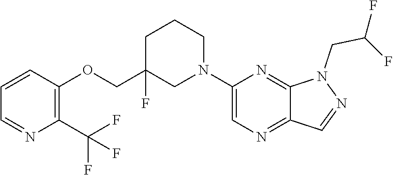 [SMILES: FC(F)Cn1ncc2ncc(N3CCCC(F)(COc4cccnc4C(F)(F)F)C3)nc21]

Step 1: tert-butyl 3-fluoro-3-({[2-(trifluoromethyl)pyridin-3-yl]oxy}methyl)piperidine-1-carboxylate: Followed General Procedure A using tert-butyl 3-fluoro-3-(hydroxymethyl)piperidine-1-carboxylate (200 mg, 0.86 mmol, 1.00 equiv) and 2-(trifluoromethyl)pyridin-3-ol (140 mg, 0.86 mmol, 1.0 equiv) to afford tert-butyl 3-fluoro-3-({[2-(trifluoromethyl)pyridin-3-yl]oxy}methyl)piperidine-1-carboxylate (190 mg, 59%) as a colorless oil. MS m/z: 379 [M+H]⁺.

Step 2: 3-[(3-fluoropiperidin-3-yl)methoxy]-2-(trifluoromethyl)pyridine hydrochloride: Followed General Procedure B using tert-butyl 3-fluoro-3-({[2-(trifluoromethyl)pyridin-3-yl]oxy}methyl)piperidine-1-carboxylate (190 mg, 0.5 mmol, 1.00 equiv) to afford 3-[(3-fluoropiperidin-3-yl)methoxy]-2-(trifluoromethyl)pyridine hydrochloride (150 mg). MS m/z: 279 [M+H]⁺.

Step 3: 3-({1-[1-(2,2-difluoroethyl)-1H-pyrazolo[3,4-b]pyrazin-6-yl]-3-fluoropiperidin-3-yl}methoxy)-2-(trifluoromethyl)pyridine: Followed General Procedure C using 3-[(3-fluoropiperidin-3-yl)methoxy]-2-(trifluoromethyl)pyridine hydrochloride (17.3 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford 3-({1-[1-(2,2-difluoroethyl)-1H-pyrazolo[3,4-b]pyrazin-6-yl]-3-fluoropiperidin-3-yl}methoxy)-2-(trifluoromethyl)pyridine (18 mg, 71%) as a white solid. ¹H NMR (500 MHZ, CDCl₃) δ 8.33 (dd, J=4.5, 1.2 Hz, 1H), 8.28 (s, 1H), 8.05 (s, 1H), 7.49 (dd, J=8.5, 4.6 Hz, 1H), 7.40 (dd, J=8.5, 1.2 Hz, 1H), 6.21 (tt, J=55.6, 4.5 Hz, 1H), 4.74-4.58 (m, 3H), 4.35-4.12 (m, 3H), 3.60 (dd, J=28.9, 14.3 Hz, 1H), 3.39-3.27 (m, 1H), 2.17-1.96 (m, 3H), 1.88-1.75 (m, 1H). MS m/z: 461 [M+H]⁺.

### (1R,5S,6S)-3-[1-(2,2-Difluoroethyl)-1H-pyrazolo[3,4-b]pyrazin-6-yl]-6-({[6-(trifluoromethyl)pyridin-2-yl]oxy}methyl)-3-azabicyclo[3.1.0]hexane (36)

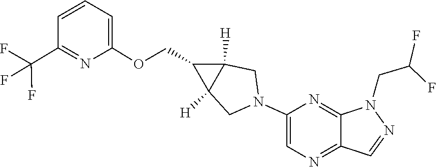 [SMILES: FC(F)Cn1ncc2ncc(N3C[C@@H]4[C@@H](COc5cccc(C(F)(F)F)n5)[C@@H]4C3)nc21]

Step 1: tert-butyl 3-fluoro-3-({[2-(trifluoromethyl)pyridin-3-yl]oxy}methyl)piperidine-1-carboxylate: Followed General Procedure D using tert-butyl (1R,5S,6S)-6-(hydroxymethyl)-3-azabicyclo[3.1.0]hexane-3-carboxylate (250 mg, 1.17 mmol, 1.00 equiv) and 2-bromo-6-(trifluoromethyl)pyridine (265 mg, 1.17 mmol, 1.0 equiv) to afford tert-butyl (1R,5S,6S)-6-({[6-(trifluoromethyl)pyridin-2-yl]oxy}methyl)-3-azabicyclo[3.1.0]hexane-3-carboxylate (360 mg, 86%) as a colorless oil. MS m/z: 359 [M+H]⁺.

Step 2: (1R,5S,6S)-6-({[6-(trifluoromethyl)pyridin-2-yl]oxy}methyl)-3-azabicyclo[3.1.0]hexane hydrochloride: Followed General Procedure B using tert-butyl (1R,5S,6S)-6-({[6-(trifluoromethyl)pyridin-2-yl]oxy}methyl)-3-azabicyclo[3.1.0]hexane-3-carboxylate (360 mg, 1 mmol, 1.00 equiv) to afford (1R,5S,6S)-6-({[6-(trifluoromethyl)pyridin-2-yl]oxy}methyl)-3-azabicyclo[3.1.0]hexane hydrochloride (250 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[6-(trifluoromethyl)pyridin-2-yl]oxy}methyl)-3-azabicyclo[3.1.0]hexane: Followed General Procedure C using (1R,5S,6S)-6-({[6-(trifluoromethyl)pyridin-2-yl]oxy}methyl)-3-azabicyclo[3.1.0]hexane hydrochloride (16.2 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford (1R,5S,6S)-3-[1-(2,2-difluoroethyl)-1H-pyrazolo[3,4-b]pyrazin-6-yl]-6-({[6-(trifluoromethyl)pyridin-2-yl]oxy}methyl)-3-azabicyclo[3.1.0]hexane (23 mg, 95%) as a white solid. ¹H NMR (500 MHz, CDCl₃) δ 8.03 (s, 1H), 7.91 (s, 1H), 7.72 (t, J=7.8 Hz, 1H), 7.25 (s, 1H), 6.93 (d, J=8.4 Hz, 1H), 6.22 (tt, J=55.7, 4.5 Hz, 1H), 4.65 (td, J=13.4, 4.5 Hz, 2H), 4.32 (d, J=7.2 Hz, 2H), 3.95 (d, J=10.7 Hz, 2H), 3.65 (dt, J=10.7, 2.1 Hz, 2H), 1.89 (d, J=3.3 Hz, 2H), 1.22 (tt, J=7.1, 3.4 Hz, 1H). MS m/z: 441 [M+H]⁺.

### 2-(1-Ethyl-5-(3-((o-tolyloxy)methyl)piperidin-1-yl)-1H-imidazo[4,5-b]pyrazin-2-yl)-1,3,4-thiadiazole (37)

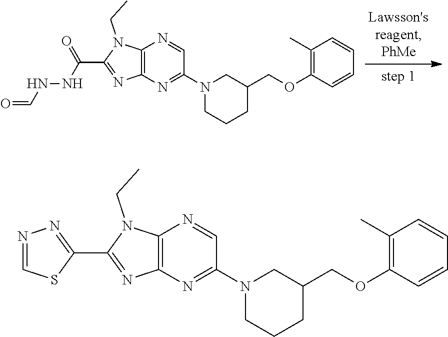 [SMILES: CCn1c(C(=O)NNC=O)nc2nc(N3CCCC(COc4ccccc4C)C3)cnc21 ; CCn1c(-c2nncs2)nc2nc(N3CCCC(COc4ccccc4C)C3)cnc21]

Step 1: 2-(1-ethyl-5-(3-((o-tolyloxy)methyl)piperidin-1-yl)-1H-imidazo[4,5-b]pyrazin-2-yl)-1,3,4-thiadiazole: A solution of 1-ethyl-N′-formyl-5-[3-(2-methylphenoxymethyl)piperidin-1-yl]imidazo[4,5-b]pyrazine-2-carbohydrazide (30 mg, 0.069 mmol, 1.00 equiv) and Lawesson reagent (16.6 mg, 0.041 mmol, 0.60 equiv) in PhCH₃ (2 mL) was stirred for 3 h at 100° C. The resulting mixture was concentrated under vacuum. The residue was purified by silica gel column chromatography, eluted with PE/EA (2:1) to afford 2-(1-ethyl-5-(3-((o-tolyloxy)methyl)piperidin-1-yl)-1H-imidazo[4,5-b]pyrazin-2-yl)-1,3,4-thiadiazole (4.5 mg, 15.1%) as a yellow solid. The residue was purified by reverse flash chromatography with the following conditions: column, silica gel; mobile phase, MeCN in water, 10% to 90% gradient in 30 min; detector, UV 254 nm. This provided 2-(1-ethyl-5-(3-((o-tolyloxy)methyl)piperidin-1-yl)-1H-imidazo[4,5-b]pyrazin-2-yl)-1,3,4-thiadiazole (4.5 mg, 15.1%) as a yellow solid. ¹H NMR (400 MHZ, CDCl₃) δ 9.25 (s, 1H), 8.42 (s, 1H), 7.19-7.11 (, 2H), 6.89-6.84 (m, 1H), 6.83-6.78 (m, 1H), 5.02 (q, J=7.1 Hz, 2H), 4.39-4.21 (m, 2H), 4.01-3.86 (m, 2H), 3.29-3.17 (m, 2H), 2.50-2.35 (m, 1H), 2.27 (s, 3H), 2.07-1.98 (m, 1H), 1.97-1.86 (m, 2H), 1.63-1.51 (m, 4H). MS m/z: 436.2 [M+H]⁺.

### 1-Methyl-5-phenyl-3-(3-((o-tolyloxy)methyl)piperidine-1-carbonyl)-1,5-dihydro-4H-pyrrolo[3,2-c]pyridin-4-one (38)

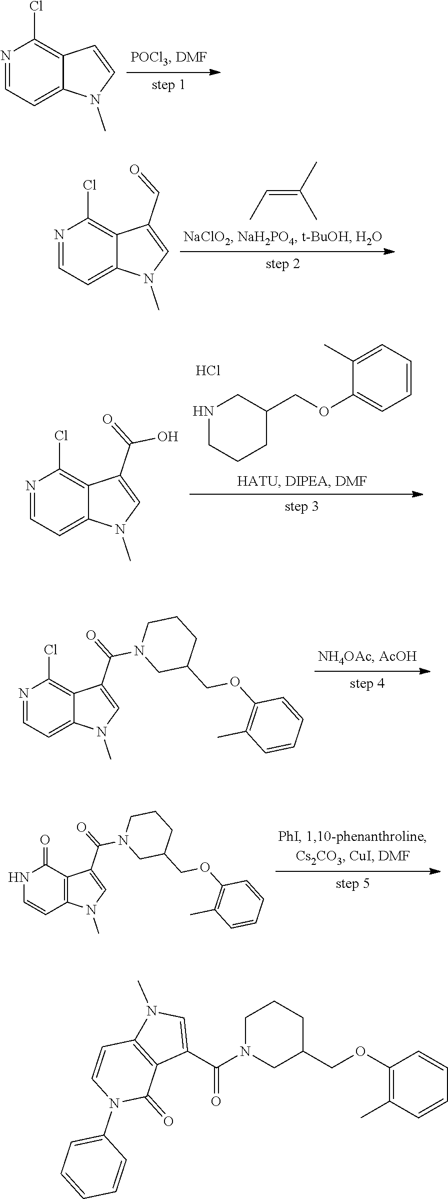

Step 1: 4-chloro-1-methyl-1H-pyrrolo[3,2-c]pyridine-3-carbaldehyde: To a solution of 4-chloro-1-methyl-1H-pyrrolo[3,2-c]pyridine (500 mg, 3.00 mmol, 1.00 equiv) in DMF (5.0 mL) was added POCl₃ (1.38 g, 9.00 mmol, 3.0 equiv) at 0° C. The resulting mixture was stirred for 2 hour at room temperature under nitrogen atmosphere. The mixture was basified to pH 10 with 1M NaOH. The resulting mixture was extracted with CH₂Cl₂ (3×30 mL). The combined organic layers were washed with water (2×30 mL), dried over anhydrous Na₂SO₄. After filtration, the filtrate was concentrated under reduced pressure. This provided 4-chloro-1-methyl-1H-pyrrolo[3,2-c]pyridine-3-carbaldehyde (350 mg, 59.9%) as a white solid. MS m/z: 195 [M+H]⁺.

Step 2: 4-chloro-1-methyl-1H-pyrrolo[3,2-c]pyridine-3-carboxylic acid: To a stirred solution of 4-chloro-1-methyl-1H-pyrrolo[3,2-c]pyridine-3-carbaldehyde (300 mg, 1.54 mmol, 1 equiv) and 2,3-dimethylbut-2-ene (324 mg, 3.85 mmol, 2.5 equiv) in t-BuOH (5 mL) and H₂O (1 mL) were added NaClO₂ (209 mg, 2.31 mmol, 1.5 equiv) and NaH₂PO₄ (1109 mg, 9.24 mmol, 6.0 equiv) in portions at 0° C. under nitrogen atmosphere. The resulting mixture was stirred for overnight at room temperature under nitrogen atmosphere. The resulting mixture was acidified to PH ˜3, and was extracted by EtOAc (3×20 mL). The combined organic layers were washed with water (2×30 mL), and brine (30 mL), dried over anhydrous Na₂SO₄. After filtration, the filtrate was concentrated under vacuum to afford 4-chloro-1-methyl-1H-pyrrolo[3,2-c]pyridine-3-carboxylic acid (250 mg, 77.0%) as a white solid. MS m/z: 211 [M+H]⁺.

Step 3: (4-chloro-1-methyl-1H-pyrrolo[3,2-c]pyridin-3-yl)(3-((o-tolyloxy)methyl)piperidin-1-yl)methanone: To a stirred solution of 4-chloro-1-methyl-1H-pyrrolo[3,2-c]pyridine-3-carboxylic acid (230 mg, 1.09 mmol, 1 equiv) and 3-((o-tolyloxy)methyl)piperidine (269 mg, 1.31 mmol, 1.2 equiv) in DMF (3 mL) were added HATU (622 mg, 1.63 mmol, 1.5 equiv) and DIPEA (423 mg, 3.27 mmol, 3 equiv) dropwise at 0° C. under nitrogen atmosphere. The resulting mixture was stirred for 2 h at room temperature under nitrogen atmosphere. The reaction was diluted with water at room temperature. The resulting mixture was extracted with EtOAc (3×10 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (5:1) to afford (4-chloro-1-methyl-1H-pyrrolo[3,2-c]pyridin-3-yl)(3-((o-tolyloxy)methyl)piperidin-1-yl)methanone (300 mg, 69.0%) as a white solid. MS m/z: 398 [M+H]⁺.

Step 4: 1-methyl-3-(3-((o-tolyloxy)methyl)piperidine-1-carbonyl)-1,5-dihydro-4H-pyrrolo[3,2-c]pyridin-4-one: To a stirred solution of (4-chloro-1-methyl-1H-pyrrolo[3,2-c]pyridin-3-yl)(3-((o-tolyloxy)methyl)piperidin-1-yl)methanone (150 mg, 0.377 mmol, 1 equiv) in AcOH (2.5 mL) was added NH₄OAc (290 mg, 3.77 mmol, 10 equiv) in portions at 0° C. under air atmosphere. The resulting mixture was stirred for overnight at 100° C. under nitrogen atmosphere. Desired product could be detected by LCMS. The resulting mixture was concentrated under reduced pressure. The residue was purified by silica gel column chromatography, eluted with PE/EA (2:1) to afford 1-methyl-3-(3-((o-tolyloxy)methyl)piperidine-1-carbonyl)-1,5-dihydro-4H-pyrrolo[3,2-c]pyridin-4-one (100 mg, 69.91%) as a white solid. MS m/z: 380 [M+H]⁺.

Step 5: 1-methyl-5-phenyl-3-(3-((o-tolyloxy)methyl)piperidine-1-carbonyl)-1,5-dihydro-4H-pyrrolo[3,2-c]pyridin-4-one: To a stirred solution of 1-methyl-3-(3-((o-tolyloxy)methyl)piperidine-1-carbonyl)-1,5-dihydro-4H-pyrrolo[3,2-c]pyridin-4-one (100 mg, 0.264 mmol, 1 equiv) and iodobenzene (107.53 mg, 0.528 mmol, 2 equiv) in DMF (3 mL) were added Cs₂CO₃ (171 mg, 0.528 mmol, 2 equiv) and CuI (5.02 mg, 0.026 mmol, 0.1 equiv), 1,10-phenanthroline (9.50 mg, 0.053 mmol, 0.2 equiv) at room temperature under air atmosphere. The resulting mixture was stirred for overnight at 100° C. under argon atmosphere. The reaction was diluted with water at room temperature. The resulting mixture was extracted with EtOAc (3×10 mL). The combined organic layers were washed with water (2×20 mL) and brine (1×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 90% gradient in 20 min; detector, UV 254 nm. This provided 1-methyl-5-phenyl-3-(3-((o-tolyloxy)methyl)piperidine-1-carbonyl)-1,5-dihydro-4H-pyrrolo[3,2-c]pyridin-4-one (35 mg, 29.1%) as a white solid. ¹H NMR (300 MHZ, DMSO-d₆) δ 7.59-7.29 (m, 6H), 7.28-6.99 (m, 3H), 6.97-6.73 (m, 2H), 6.69 (d, J=7.4 Hz, 1H), 4.62-4.19 (m, 1H), 4.01-3.81 (m, 2H), 3.73 (d, J=18.3 Hz, 3H), 3.61-3.48 (m, 1H), 3.11-2.72 (m, 2H), 2.18 (s, 1H), 2.04 (d, J=25.5 Hz, 1H), 1.88-1.18 (m, 6H). MS m/z: 456.0 [M+H]⁺.

### 5-Methyl-6-phenyl-3-(3-((o-tolyloxy)methyl)pyrrolidin-1-yl)-5H-pyrrolo[2,3-b]pyrazine (39)

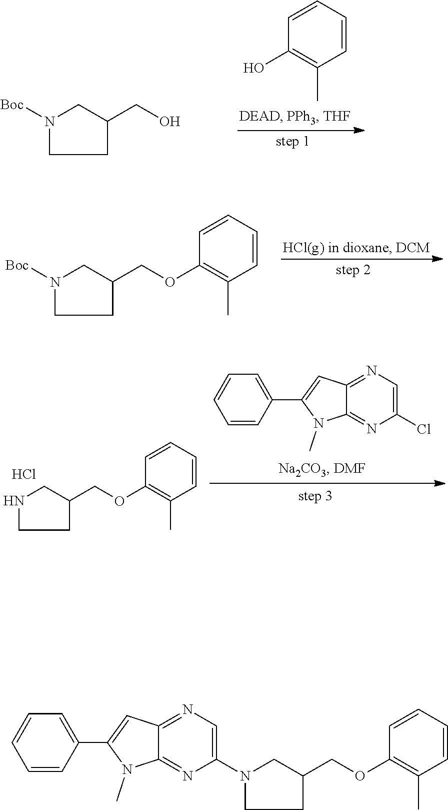

Step 1: tert-butyl 3-((o-tolyloxy)methyl)pyrrolidine-1-carboxylate: To a stirred mixture of o-cresol (200 mg, 1.85 mmol, 1 equiv), tert-butyl 3-(hydroxymethyl)pyrrolidine-1-carboxylate (447 mg, 2.22 mmol, 1.2 equiv) in THF (5 mL) and PPh₃ (728 mg, 2.77 mmol, 1.5 equiv) in THF (4 mL) was added TMAD (478 mg, 2.77 mmol, 1.5 equiv) in portion at 0° C. The resulting mixture was warmed to 50° C. and stirred for overnight. Desired product could be detected by LCMS. The resulting mixture was concentrated under reduced pressure. The residue was purified by silica gel column chromatography, eluted with EtOAc/PE (1/1) to afford tert-butyl 3-((o-tolyloxy)methyl)pyrrolidine-1-carboxylate (120 mg, 22.3%) as a yellow solid. MS m/z: 292 [M+H]⁺.

Step 2: 3-((o-tolyloxy)methyl)pyrrolidine hydrochloride: To a stirred solution of tert-butyl 3-(2-methylphenoxymethyl)pyrrolidine-1-carboxylate (120 mg, 0.412 mmol, 1 equiv) in DCM (2.5 mL) was added HCl (gas) in dioxane (2.5 mL). The mixture was stirred at room temperature for 2 h. After removing the solvent, the crude product 3-((o-tolyloxy)methyl)pyrrolidine hydrochloride (100 mg) was directly used in next step without further purification. MS m/z: 192 [M+H]⁺.

Step 3: 5-methyl-6-phenyl-3-(3-((o-tolyloxy)methyl)pyrrolidin-1-yl)-5H-pyrrolo[2,3-b]pyrazine: To a stirred solution of 3-((o-tolyloxy)methyl)pyrrolidine (100 mg, 0.410 mmol, 1 equiv) and 3-(2-methylphenoxymethyl)pyrrolidine (86.3 mg, 0.451 mmol, 1.1 equiv) in DMF (1.00 mL) was added Na₂CO₃ (87.0 mg, 0.820 mmol, 2 equiv) at room temperature. The resulting mixture was stirred for 2 h at 100° C. under nitrogen atmosphere. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 5-methyl-6-phenyl-3-(3-((o-tolyloxy)methyl)pyrrolidin-1-yl)-5H-pyrrolo[2,3-b]pyrazine (13 mg, 10.40%) as an white solid. ¹H NMR (400 MHZ, DMSO-d₆) δ 7.87 (s, 1H), 7.63 (d, J=7.6 Hz, 2H), 7.51 (t, J=7.5 Hz, 2H), 7.42 (t, J=7.3 Hz, 1H), 7.14 (d, J=7.4 Hz, 2H), 6.96 (d, J=8.1 Hz, 1H), 6.84 (t, J=7.4 Hz, 1H), 6.60 (s, 1H), 4.06 (d, J=6.5 Hz, 2H), 3.79 (dd, J=10.5, 7.5 Hz, 1H), 3.72 (s, 4H), 3.56 (q, J=8.2 Hz, 1H), 3.45 (dd, J=10.6, 6.6 Hz, 1H), 2.85 (t, J=7.1 Hz, 1H), 2.17 (s, 4H), 1.98 (t, J=12.7, 7.2 Hz, 1H). MS m/z: 398.9 [M+H]⁺.

### (1R,5S,6S)-3-[6-(1,3,4-Thiadiazol-2-yl)pyrazin-2-yl]-6-({[2-(trifluoromethyl)pyridin-3-yl]oxy}methyl)-3-azabicyclo[3.1.0]hexane (40)

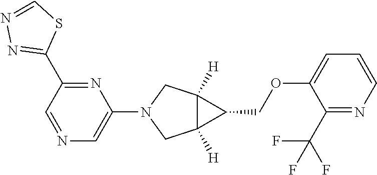 [SMILES: FC(F)(F)c1ncccc1OC[C@@H]1[C@H]2CN(c3cncc(-c4nncs4)n3)C[C@@H]12]

Followed General Procedure C using (1R,5S,6S)-6-({[2-(trifluoromethyl)pyridin-3-yl]oxy}methyl)-3-azabicyclo[3.1.0]hexane hydrochloride (16.2 mg, 0.055 mmol, 1.00 equiv) and 2-chloro-6-(1,3,4-thiadiazol-2-yl)pyrazine (11 mg, 0.055 mmol, 1.1 equiv) to afford (1R,5S,6S)-3-[6-(1,3,4-thiadiazol-2-yl)pyrazin-2-yl]-6-({[2-(trifluoromethyl)pyridin-3-yl]oxy}methyl)-3-azabicyclo[3.1.0]hexane (13 mg, 56%) as a white solid. ¹H NMR (500 MHz, CDCl₃) δ 9.19 (s, 1H), 8.85 (s, 1H), 8.28 (dd, J=4.6, 1.2 Hz, 1H), 7.97 (s, 1H), 7.46 (dd, J=8.5, 4.6 Hz, 1H), 7.37 (d, J=8.5 Hz, 1H), 4.13 (d, J=6.2 Hz, 2H), 3.90 (d, J=10.4 Hz, 2H), 3.63 (dt, J=10.6, 1.9 Hz, 2H), 1.97-1.89 (m, 2H), 1.28-1.25 (m, 1H). MS m/z: 421 [M+H]⁺.

### 1-(4-{5-Methyl-2-[3-(phenoxymethyl)piperidin-1-yl]-5H-pyrrolo[2,3-b]pyrazin-6-yl}piperidin-1-yl)ethan-1-one (41)

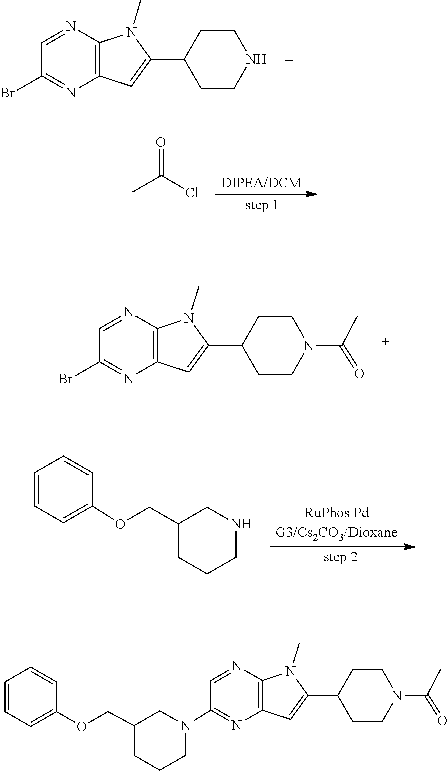

Step 1: 1-(4-{2-bromo-5-methyl-5H-pyrrolo[2,3-b]pyrazin-6-yl}piperidin-1-yl)ethan-1-one: 4-{2-bromo-5-methyl-5H-pyrrolo[2,3-b]pyrazin-6-yl}piperidine (300 mg, 1.02 mmol), DIPEA (266 μL, 1.5 eq., 1.52 mmol) and acetyl chloride (87.0 μL, 1.2 eq., 1.22 mmol) were combined with DCM (4 ml). The reaction mixture was stirred overnight. The crude reaction mixture was concentrated under vacuum and purified by chromatography (silica gel, 12 g. EtOAc/hexane=50:50 to 100:0). The fractions were concentrated and dried under vacuum to afford 1-(4-{2-bromo-5-methyl-5H-pyrrolo[2,3-b]pyrazin-6-yl}piperidin-1-yl)ethan-1-one as a white solid (310, 90%). MS m/z: 338 [M+H]⁺.

Step 2: 1-(4-{5-methyl-2-[3-(phenoxymethyl)piperidin-1-yl]-5H-pyrrolo[2,3-b]pyrazin-6-yl}piperidin-1-yl)ethan-1-one: A mixture of 1-(4-{2-bromo-5-methyl-5H-pyrrolo[2,3-b]pyrazin-6-yl}piperidin-1-yl)ethan-1-one (14.8 mg, 43.9 μmol) and 1-(4-{2-bromo-5-methyl-5H-pyrrolo[2,3-b]pyrazin-6-yl}piperidin-1-yl)ethan-1-one (14.8 mg, 43.9 μmol), and dicesium(1+) carbonate (42.9 mg, 3 eq., 132 μmol), RuPhos Pd G3 (3.67 mg, 0.1 eq., 4.39 μmol) in 1,4-dioxane (500 μL) was heated at 70° C. overnight. The reaction was monitored by LCMS. The mixture was cooled to rt and filtered through Celite. The filtrate was concentrated and purified by flash chromatography (silica gel, 12 g. EtOAc/hexane=50:50 to 100:0) to afford 1-(4-{5-methyl-2-[3-(phenoxymethyl)piperidin-1-yl]-5H-pyrrolo[2,3-b]pyrazin-6-yl}piperidin-1-yl)ethan-1-one as a colorless oil (5 mg, 25%). ¹H NMR (500 MHZ, DMSO-d₆) δ 7.95 (s, 1H), 7.32-7.24 (m, 2H), 6.99-6.89 (m, 3H), 6.15 (s, 1H), 4.52 (d, J=12.9 Hz, 1H), 4.24 (dd, J=12.7, 3.8 Hz, 1H), 4.06 (d, J=12.7 Hz, 1H), 3.97-3.88 (m, 3H), 3.73 (s, 3H), 3.20 (td, J=13.1, 2.5 Hz, 2H), 3.16-3.03 (m, 2H), 2.92-2.83 (m, 1H), 2.75 (dd, J=12.7, 10.2 Hz, 1H), 2.67 (td, J=13.2, 3.0 Hz, 1H), 2.04 (s, 4H), 2.00-1.84 (m, 5H), 1.76 (dt, J=13.1, 3.6 Hz, 1H), 1.64-1.53 (m, 2H), 1.49-1.39 (m, 1H), 1.39-1.29 (m, 1H). MS m/z: 448 [M+H]⁺.

### 2-({1-[1-(2,2-Difluoroethyl)-1H-pyrazolo[3,4-b]pyrazin-6-yl]-4,4-difluoropiperidin-3-yl}methoxy)-6-(trifluoromethyl)pyridine (42)

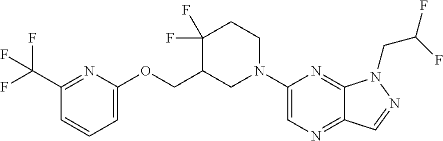 [SMILES: FC(F)Cn1ncc2ncc(N3CCC(F)(F)C(COc4cccc(C(F)(F)F)n4)C3)nc21]

Step 1: tert-butyl 4,4-difluoro-3-({[6-(trifluoromethyl)pyridin-2-yl]oxy}methyl)piperidine-1-carboxylate: Followed General Procedure D using tert-butyl 4,4-difluoro-3-(hydroxymethyl)piperidine-1-carboxylate (120 mg, 0.48 mmol, 1.00 equiv) and 2-bromo-6-(trifluoromethyl)pyridine (108 mg, 0.48 mmol, 1.0 equiv) to afford tert-butyl 4,4-difluoro-3-({[6-(trifluoromethyl)pyridin-2-yl]oxy}methyl)piperidine-1-carboxylate (181 mg, 96%) as a colorless oil. MS m/z: 397 [M+H]⁺.

Step 2: 2-[(4,4-difluoropiperidin-3-yl)methoxy]-6-(trifluoromethyl)pyridine hydrochloride: Followed General Procedure B using tert-butyl 4,4-difluoro-3-({[6-(trifluoromethyl)pyridin-2-yl]oxy}methyl)piperidine-1-carboxylate (181 mg, 1 mmol, 1.00 equiv) to afford 2-[(4,4-difluoropiperidin-3-yl)methoxy]-6-(trifluoromethyl)pyridine hydrochloride (150 mg). MS m/z: 297 [M+H]⁺.

Step 3: 2-({1-[1-(2,2-difluoroethyl)-1H-pyrazolo[3,4-b]pyrazin-6-yl]-4,4-difluoropiperidin-3-yl}methoxy)-6-(trifluoromethyl)pyridine: Followed General Procedure C using 2-[(4,4-difluoropiperidin-3-yl)methoxy]-6-(trifluoromethyl)pyridine hydrochloride (18.3 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford 2-({1-[1-(2,2-difluoroethyl)-1H-pyrazolo[3,4-b]pyrazin-6-yl]-4,4-difluoropiperidin-3-yl}methoxy)-6-(trifluoromethyl)pyridine (17 mg, 65%) as a colorless oil. ¹H NMR (500 MHZ, CDCl₃) δ 8.28 (s, 1H), 8.05 (s, 1H), 7.78-7.72 (m, 1H), 7.29 (d, J=7.3 Hz, 1H), 6.96 (d, J=8.4 Hz, 1H), 6.19 (tt, J=55.5, 4.5 Hz, 1H), 4.79 (dd, J=11.3, 4.0 Hz, 1H), 4.62 (td, J=13.3, 4.5 Hz, 2H), 4.48 (d, J=14.0 Hz, 1H), 4.43 (dd, J=11.3, 8.9 Hz, 1H), 4.31 (dd, J=15.3, 3.8 Hz, 1H), 3.64 (ddd, J=14.0, 10.4, 3.6 Hz, 1H), 3.54 (ddd, J=13.9, 9.6, 1.5 Hz, 1H), 2.65-2.56 (m, 1H), 2.31-2.23 (m, 1H), 2.16-2.00 (m, 1H). MS m/z: 479 [M+H]⁺.

### 2-({1-[1-(2,2-Difluoroethyl)-1H-pyrazolo[3,4-b]pyrazin-6-yl]-3-fluoropiperidin-3-yl}methoxy)-6-(trifluoromethyl)pyridine (43)

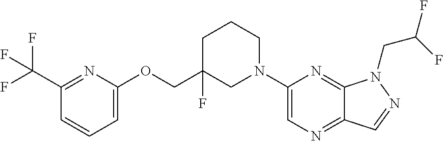 [SMILES: FC(F)Cn1ncc2ncc(N3CCCC(F)(COc4cccc(C(F)(F)F)n4)C3)nc21]

Step 1: tert-butyl 3-fluoro-3-({[6-(trifluoromethyl)pyridin-2-yl]oxy}methyl)piperidine-1-carboxylate: Followed General Procedure D using tert-butyl 3-fluoro-3-(hydroxymethyl)piperidine-1-carboxylate (111 mg, 0.48 mmol, 1.00 equiv) and 2-bromo-6-(trifluoromethyl)pyridine (108 mg, 0.48 mmol, 1.0 equiv) to afford tert-butyl 3-fluoro-3-({[6-(trifluoromethyl)pyridin-2-yl]oxy}methyl)piperidine-1-carboxylate (177 mg, 98%) as a colorless oil. MS m/z: 379 [M+H]⁺.

Step 2: 2-[(4,4-difluoropiperidin-3-yl)methoxy]-6-(trifluoromethyl)pyridine hydrochloride: Followed General Procedure B using tert-butyl 3-fluoro-3-({[6-(trifluoromethyl)pyridin-2-yl]oxy}methyl)piperidine-1-carboxylate (177 mg, 1 mmol, 1.00 equiv) to afford 2-[(3-fluoropiperidin-3-yl)methoxy]-6-(trifluoromethyl)pyridine hydrochloride (160 mg). MS m/z: 279 [M+H]⁺.

Step 3: 2-({1-[1-(2,2-difluoroethyl)-1H-pyrazolo[3,4-b]pyrazin-6-yl]-3-fluoropiperidin-3-yl}methoxy)-6-(trifluoromethyl)pyridine: Followed General Procedure C using 2-[(3-fluoropiperidin-3-yl)methoxy]-6-(trifluoromethyl)pyridine hydrochloride (17.3 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford 2-({1-[1-(2,2-difluoroethyl)-1H-pyrazolo[3,4-b]pyrazin-6-yl]-3-fluoropiperidin-3-yl}methoxy)-6-(trifluoromethyl)pyridine (23 mg, 91%) as a colorless oil. ¹H NMR (500 MHz, CDCl₃) δ 8.27 (s, 1H), 8.03 (s, 1H), 7.77 (t, J=7.9 Hz, 1H), 7.30 (d, J=7.3 Hz, 1H), 7.05 (d, J=8.4 Hz, 1H), 6.21 (tt, J=55.6, 4.5 Hz, 1H), 4.70-4.61 (m, 2H), 4.57 (s, 1H), 4.53 (d, J=3.7 Hz, 1H), 4.45 (dd, J=14.2, 8.7 Hz, 1H), 4.28 (dt, J=12.9, 4.3 Hz, 1H), 3.61 (dd, J=27.1, 14.2 Hz, 1H), 3.41-3.33 (m, 1H), 2.20-2.11 (m, 1H), 2.10-1.86 (m, 2H), 1.86-1.75 (m, 1H). MS m/z: 461 [M+H]⁺.

### 4-(2-Oxo-2-(3-(phenoxymethyl)piperidin-1-yl)ethyl)-2-phenylpyridazin-3(2H)-one (44)

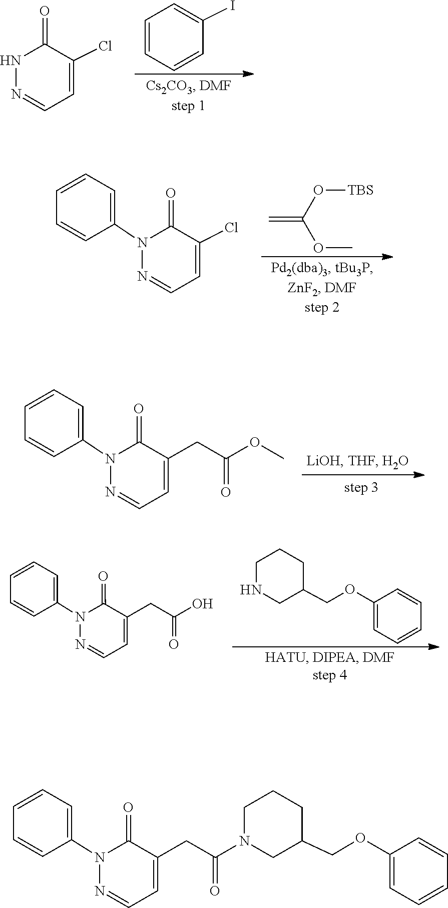

Step 1: 4-chloro-2-phenylpyridazin-3(2H)-one: To a stirred solution of 4-chloropyridazin-3(2H)-one (1 g, 7.661 mmol, 1 equiv) and iodobenzene (3.13 g, 15.3 mmol, 2 equiv) in DMF (10 mL) were added Cs₂CO₃ (7.49 g. 22.9 mmol, 3 equiv) and CuI (0.15 g, 0.766 mmol, 0.1 equiv), 1,10-phenanthroline (0.14 g, 0.766 mmol, 0.1 equiv) at room temperature under air atmosphere. The resulting mixture was stirred for overnight at 100° C. under argon atmosphere. The reaction was quenched with Water at room temperature. The resulting mixture was extracted with EtOAc (3×10 mL). The combined organic layers were washed with brine (1×6 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 50% gradient in 10 min; detector, UV 254 nm. This provided 4-chloro-2-phenylpyridazin-3(2H)-one (600 mg, 37.9%) as a white solid. MS m/z: 207 [M+H]⁺.

Step 2: methyl 2-(3-oxo-2-phenyl-2,3-dihydropyridazin-4-yl)acetate: To a stirred solution of 4-chloro-2-phenylpyridazin-3(2H)-one (580 mg, 2.80 mmol, 1 equiv) and tert-butyl[(1-methoxyethenyl)oxy]dimethylsilane (634 mg, 3.36 mmol, 1.2 equiv) in DMF (6 mL) were added Pd₂(dba)₃ (257 mg, 0.281 mmol, 0.1 equiv), zinc fluoride (290 mg, 2.80 mmol, 1 equiv) and tri-tert-butylphosphine (113 mg, 0.561 mmol, 0.2 equiv) at room temperature under air atmosphere. The resulting mixture was stirred for 16 h at 100° C. under argon atmosphere. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (5:1) to afford methyl 2-(3-oxo-2-phenyl-2,3-dihydropyridazin-4-yl)acetate (350 mg, 51.0%) as a white solid. MS m/z: 245 [M+H]⁺.

Step 3: 2-(3-oxo-2-phenyl-2,3-dihydropyridazin-4-yl)acetic acid: To a stirred solution of methyl 2-(3-oxo-2-phenyl-2,3-dihydropyridazin-4-yl)acetate (350 mg, 1.433 mmol, 1 equiv) in THF (2 mL) and H₂O (2 mL) was added LiOH (41.1 mg, 1.72 mmol, 1.2 equiv) dropwise at 0° C. under air atmosphere. The resulting mixture was stirred for 2 h at room temperature under nitrogen atmosphere. The resulting mixture was concentrated under vacuum. The resulting mixture was concentrated under vacuum to afford 2-(3-oxo-2-phenyl-2,3-dihydropyridazin-4-yl)acetic acid (250 mg, 75.8%) as a white solid. MS m/z: 231 [M+H]⁺.

Step 4: 4-(2-oxo-2-(3-(phenoxymethyl)piperidin-1-yl)ethyl)-2-phenylpyridazin-3(2H)-one: To a stirred solution of 2-(3-oxo-2-phenyl-2,3-dihydropyridazin-4-yl)acetic acid (100 mg, 0.434 mmol, 1 equiv) and 3-(phenoxymethyl)piperidine (124 mg, 0.651 mmol, 1.5 equiv) in DMF (3 mL) were added HATU (247 mg, 0.651 mmol, 1.5 equiv) and DIPEA (168 mg. 1.30 mmol, 3 equiv) dropwise at 0° C. under nitrogen atmosphere. The resulting mixture was stirred for 2 h at room temperature under nitrogen atmosphere. The reaction was quenched with Water at room temperature. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (5:1) to afford 4-(2-oxo-2-(3-(phenoxymethyl)piperidin-1-yl)ethyl)-2-phenylpyridazin-3(2H)-one (20 mg, 11.4%) as a white solid. ¹H NMR (300 MHz, DMSO-d₆) δ 8.03-7.97 (m, 1H), 7.57-7.39 (m, 5H), 7.39-7.21 (m, 3H), 6.99-6.86 (m, 3H), 4.41-3.99 (m, 1H), 3.98-3.69 (m, 3H), 3.65 (d, J=8.0 Hz, 2H), 3.21-3.06 (m, 1H), 2.91-2.62 (m, 1H), 1.93 (d, J=40.2 Hz, 2H), 1.73 (d, J=12.8 Hz, 1H), 1.40 (d, J=9.2 Hz, 2H). MS m/z: 404.1 [M+H]⁺.

### 2-(6-((S)-3-((S)-1-((2-(Trifluoromethyl)pyridin-3-yl)oxy)ethyl)piperidin-1-yl)pyrazin-2-yl)-1,3,4-thiadiazole (45a) & 2-(6-((S)-3-((R)-1-((2-(trifluoromethyl)pyridin-3-yl)oxy)ethyl)piperidin-1-yl)pyrazin-2-yl)-1,3,4-thiadiazole (45b)

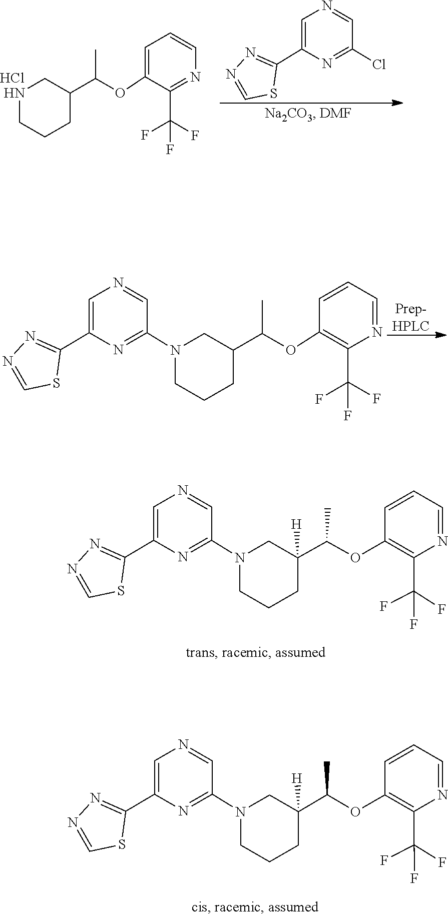

Step 1: 2-(6-(3-(1-((2-(trifluoromethyl)pyridin-3-yl)oxy)ethyl)piperidin-1-yl)pyrazin-2-yl)-1,3,4-thiadiazole: Followed General Procedure C using 3-(1-(piperidin-3-yl)ethoxy)-2-(trifluoromethyl)pyridine hydrochloride (20 mg, 0.064 mmol, 1.00 equiv) and 2-chloro-6-(1,3,4-thiadiazol-2-yl)pyrazine (14 mg, 0.07 mmol, 1.1 equiv). The crude product was purified by reverse flash chromatography with the following conditions: column, silica gel; mobile phase, MeCN in water, 10% to 50% gradient in 10 min; detector, UV 254 nm. This provided 2-(6-(3-(1-((2-(trifluoromethyl)pyridin-3-yl)oxy)ethyl)piperidin-1-yl)pyrazin-2-yl)-1,3,4-thiadiazole (35 mg) as a yellow solid. This product was further purified by prep. HPLC with the following conditions: Column: YMC-Actus Triart C18, 30*150 mm, 5 μm; Mobile Phase A: Water (0.1% FA), Mobile Phase B: ACN; Flow rate: 60 mL/min; Gradient: 42% B to 55% B in 12 min, 55% B; Wave Length: 254/220 nm; RT1(min): 10.63/11.3. This provided 2-(6-((S)-3-((S)-1-((2-(trifluoromethyl)pyridin-3-yl)oxy)ethyl)piperidin-1-yl)pyrazin-2-yl)-1,3,4-thiadiazole (45a, 19.2 mg, 54.8%) and 2-(6-((S)-3-((R)-1-((2-(trifluoromethyl)pyridin-3-yl)oxy)ethyl)piperidin-1-yl)pyrazin-2-yl)-1,3,4-thiadiazole (45b, 7 mg, 20%) as yellow solids. 45a: ¹H NMR (400 MHZ, Methanol-d₄): δ 9.50 (s, 1H), 8.60 (s, 1H), 8.33 (s, 1H), 8.18 (m, 1H), 7.76 (m, 1H), 7.60 (m, 1H), 4.72 (m, 1H), 4.54-4.37 (m, 2H), 3.14-3.06 (m, 1H), 3.09-3.00 (m, 1H), 2.13-2.07 (m, 1H), 2.06-1.85 (m, 2H), 1.73-1.56 (m, 2H), 1.41 (d, J=6.2 Hz, 3H). MS m/z: 437.1 [M+H]⁺. 45b: ¹H NMR (300 MHZ, DMSO-d₆): ¹H NMR (400 MHZ, Methanol-d₄): δ 9.48 (s, 1H), 8.57 (s, 1H), 8.30 (s, 1H), 8.13 (m, 1H), 7.67 (m, 1H), 7.53 (m, 1H), 4.65 (m, 1H), 4.46-4.37 (m, 1H), 4.29-4.19 (m, 1H), 3.31-3.20 (m, 2H), 2.01-1.93 (m, 2H), 1.93-1.84 (m, 1H), 1.74-1.59 (m, 2H), 1.38 (d, J=6.1 Hz, 3H). MS m/z:437.2 [M+H]⁺.

### (3-(Phenoxymethyl)piperidin-1-yl)(4-(5-phenyl-1,3,4-oxadiazol-2-yl)tetrahydro-2H-pyran-4-yl)methanone (46)

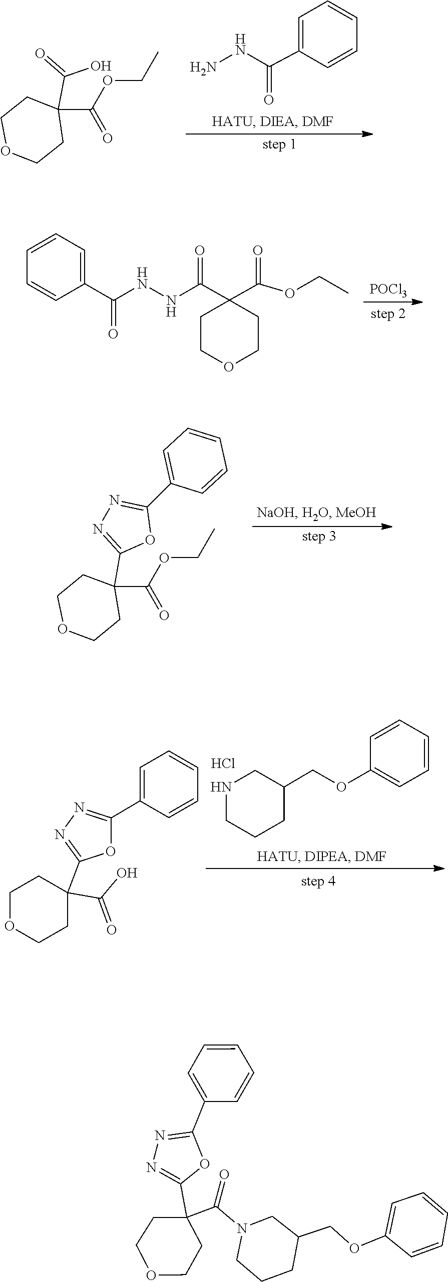

Step 1: ethyl 4-(2-benzoylhydrazine-1-carbonyl)tetrahydro-2H-pyran-4-carboxylate: To a stirred solution of benzohydrazide (500 mg, 3.67 mmol, 1 equiv) and 4-(ethoxycarbonyl)oxane-4-carboxylic acid (891 mg, 4.41 mmol, 1.2 equiv) in DCM (11 mL) were added HATU (2094 mg, 5.51 mmol, 1.5 equiv) and DIPEA (712 mg, 5.51 mmol, 1.5 equiv) dropwise at 0° C. The resulting mixture was stirred for 3 hours at room temperature. The residue was purified by silica gel column chromatography, eluted with PE/EA (1:1) to afford ethyl 4-(2-benzoylhydrazine-1-carbonyl)tetrahydro-2H-pyran-4-carboxylate (660 mg, 56.10%) as a light yellow oil. MS m/z: 321 [M+H]⁺.

Step 2: ethyl 4-(5-phenyl-1,3,4-oxadiazol-2-yl)tetrahydro-2H-pyran-4-carboxylate: A solution of ethyl 4-(N′-benzoylhydrazinecarbonyl)oxane-4-carboxylate (660 mg, 2.06 mmol, 1 equiv) in POCl₃ (10.08 mL, 65.7 mmol, 25 equiv) was stirred for 2 hours at 100° C. The residue was quenched by water at 0° C. The resulting mixture was extracted with EtOAc (3×30 mL). The combined organic layers were washed with water (2×10 mL) and brine (1×20 mL), dried over anhydrous Na₂SO₄. After filtration, the filtrate was concentrated under reduced pressure. The residue was purified by silica gel column chromatography, eluted with PE/EA (1:2) to afford ethyl 4-(5-phenyl-1,3,4-oxadiazol-2-yl)tetrahydro-2H-pyran-4-carboxylate (200 mg, 32.11%) as a white solid. MS m/z: 303 [M+H]⁺.

Step 3: 4-(5-phenyl-1,3,4-oxadiazol-2-yl)tetrahydro-2H-pyran-4-carboxylic acid: To the solution of ethyl 4-(5-phenyl-1,3,4-oxadiazol-2-yl)oxane-4-carboxylate (200 mg, 0.662 mmol, 1 equiv) in MeOH (2 mL) was added NaOH (39.69 mg, 0.993 mmol, 1.5 equiv) in water (1.00 mL). The mixture was stirred at room temperature for 1 h. The mixture was concentrated by 3M HCl aq. The aqueous phase was extracted by EtOAc (3×10 mL). The combined organic layers were washed by water (2×15 mL) and brine (1×15 mL), dried over anhydrous Na₂SO₄. After filtration, the resulting mixture was concentrated under reduced pressure to afford 4-(5-phenyl-1,3,4-oxadiazol-2-yl)tetrahydro-2H-pyran-4-carboxylic acid (150 mg, 82.67%) as a white solid. MS m/z: 275 [M+H]⁺.

Step 4: (3-(phenoxymethyl)piperidin-1-yl)(4-(5-phenyl-1,3,4-oxadiazol-2-yl)tetrahydro-2H-pyran-4-yl)methanone: To a stirred solution of 4-(5-phenyl-1,3,4-oxadiazol-2-yl)oxane-4-carboxylic acid (150 mg, 0.547 mmol, 1 equiv) and 3-(phenoxymethyl)piperidine (125.53 mg, 0.656 mmol, 1.2 equiv) in DMF (3 mL) were added HATU (311.92 mg, 0.821 mmol, 1.5 equiv) and DIPEA (106.03 mg, 0.821 mmol, 1.5 equiv) dropwise at 0° C. The resulting mixture was stirred for additional 3 h at room temperature. The resulting mixture was diluted with water (10 mL). The resulting mixture was extracted with EtOAc (3×15 mL). The combined organic layers were washed with brine (3×10 mL), dried over anhydrous Na₂SO₄. After filtration, the filtrate was concentrated under reduced pressure. The residue was purified by reverse-phase Combi-Flash with the following conditions: column, C18 gel; mobile phase, MeCN in water (0.1% FA), 20% to 70% gradient in 16 min; detector, UV 254 nm. This provided (3-(phenoxymethyl)piperidin-1-yl)(4-(5-phenyl-1,3,4-oxadiazol-2-yl)tetrahydro-2H-pyran-4-yl)methanone (30 mg, 11.49%) as a white solid. ¹H NMR (400 MHZ, DMSO-d₆) δ 8.02-7.95 (m, 2H), 7.68-7.53 (m, 3H), 7.28-7.21 (m, 2H), 6.89 (dd, J=22.6, 7.6 Hz, 3H), 3.98-3.43 (m, 8H), 2.81 (dd, J=63.5, 14.6 Hz, 2H), 2.30-2.17 (m, 4H), 1.70 (s, 2H), 1.59 (s, 1H), 1.23 (s, 2H). MS m/z: 448.1 [M+H]⁺.

### 1-(2,2-Difluoroethyl)-6-((3R,5S)-3-methyl-5-(((2-(trifluoromethyl)pyridin-3-yl)oxy)methyl)piperidin-1-yl)-1H-pyrazolo[3,4-b]pyrazine (47a) and 1-(2,2-difluoroethyl)-6-((3S,5S)-3-methyl-5-(((2-(trifluoromethyl)pyridin-3-yl)oxy)methyl)piperidin-1-yl)-1H-pyrazolo[3,4-b]pyrazine (47b)

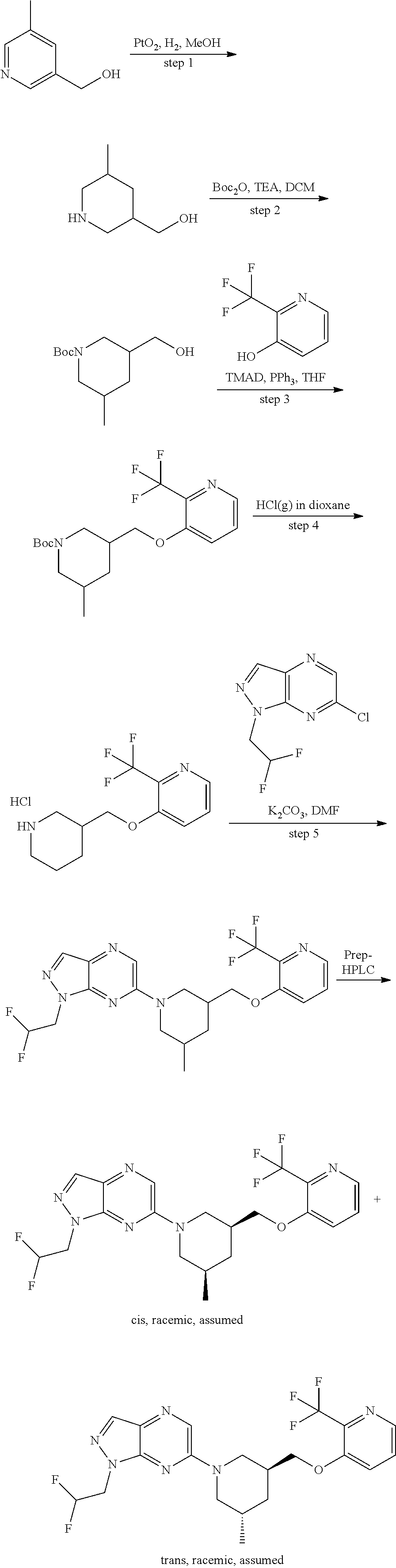

Step 1: (5-methylpiperidin-3-yl)methanoltert-butyl: To a solution of (5-methylpyridin-3-yl)methanol (1 g, 8.12 mmol, 1 equiv) in MeOH (10 mL) and HCl(6M) (1 mL) was added PtO₂ (0.37 g, 1.62 mmol, 0.2 equiv) in a pressure tank. The mixture was hydrogenated at room temperature under 30 psi of hydrogen pressure for overnight, filtered through a Celite pad and concentrated under reduced pressure. This provided (5-methylpiperidin-3-yl)methanol (600 mg, curde) as a white solid. MS m/z: 130 [M+H]⁺.

Step 2: tert-butyl 3-(hydroxymethyl)-5-methylpiperidine-1-carboxylate: To a stirred solution of (5-methylpiperidin-3-yl)methanol (0.60 g, 4.64 mmol, 1.00 equiv) in DCM (5.0 mL) were added Boc₂O (1.52 g, 6.96 mmol, 1.5 equiv) and TEA (1.41 g, 13.9 mmol, 3.0 equiv), The resulting mixture was stirred for 2 h at room temperature under nitrogen atmosphere. Desired product could be detected by LCMS. The resulting mixture was concentrated under reduced pressure. The residue was purified by silica gel column chromatography, eluted with PE/EA (8:1) to afford tert-butyl 3-(hydroxymethyl)-5-methylpiperidine-1-carboxylate (450 mg, 42.2%) as a colorless oil. MS m/z: 230 [M+H]⁺.

Step 3: tert-butyl 3-methyl-5-(((2-(trifluoromethyl)pyridin-3-yl)oxy)methyl)piperidine-1-carboxylate: To a stirred solution of tert-butyl 3-(hydroxymethyl)-5-methylpiperidine-1-carboxylate (400 mg, 1.74 mmol, 1.00 equiv) and 2-(trifluoromethyl)pyridin-3-ol (426 mg, 2.61 mmol, 1.5 equiv) in THF (5 mL) were added PPh₃ (732 mg, 2.79 mmol, 1.6 equiv) and TMAD (480 mg, 2.79 mmol, 1.6 equiv), The resulting mixture was stirred for overnight at room temperature under nitrogen atmosphere. Desired product could be detected by LCMS. The resulting mixture was concentrated under reduced pressure. The residue was purified by silica gel column chromatography, eluted with PE/EA (8:1) to afford tert-butyl 3-methyl-5-(((2-(trifluoromethyl)pyridin-3-yl)oxy)methyl)piperidine-1-carboxylate (200 mg, 30.6%) as a colorless oil. MS m/z: 375 [M+H]⁺.

Step 4: 3-((5-methylpiperidin-3-yl)methoxy)-2-(trifluoromethyl)pyridine hydrochloride: To a stirred solution of 3-methyl-5-(((2-(trifluoromethyl)pyridin-3-yl)oxy)methyl)piperidine-1-carboxylate (200 mg, 0.534 mmol, 1.00 equiv) in DCM (3.0 mL) were added HCl (gas) in 1,4-dioxane (3.0 mL) dropwise at room temperature under nitrogen atmosphere. The resulting mixture was concentrated under vacuum. This provided 3-((5-methylpiperidin-3-yl)methoxy)-2-(trifluoromethyl)pyridine hydrochloride (310 mg, crude) as a white solid. MS m/z: 275 [M+H]⁺.

Step 5: 1-(2,2-difluoroethyl)-6-((3R,5S)-3-methyl-5-(((2-(trifluoromethyl)pyridin-3-yl)oxy)methyl)piperidin-1-yl)-1H-pyrazolo[3,4-b]pyrazine (assumed); 1-(2,2-difluoroethyl)-6-((3S,5S)-3-methyl-5-(((2-(trifluoromethyl)pyridin-3-yl)oxy)methyl)piperidin-1-yl)-1H-pyrazolo[3,4-b]pyrazine (assumed): To a stirred solution of 3-((5-methylpiperidin-3-yl)methoxy)-2-(trifluoromethyl)pyridine hydrochloride (120 mg, 0.437 mmol, 1.00 equiv) and 6-chloro-1-(2,2-difluoroethyl)-1H-pyrazolo[3,4-b]pyrazine (95.6 mg, 0.437 mmol, 1 equiv) in DMF (3 mL) was added Na₂CO₃ (139 mg, 1.31 mmol, 3 equiv) at 100° C. under nitrogen atmosphere. The residue was purified by reverse flash chromatography with the following conditions: column, C18 silica gel; mobile phase, ACN in water, 10% to 90% gradient in 10 min; detector, UV 254 nm. This provided 1-(2,2-difluoroethyl)-6-(3-methyl-5-(((2-(trifluoromethyl)pyridin-3-yl)oxy)methyl)piperidin-1-yl)-1H-pyrazolo[3,4-b]pyrazine (70 mg) as a white solid. This product was further purified by prep. HPLC with the following conditions: Column: YMC-Actus Triart C18, 30*150 mm, 5 um; Mobile Phase A: Water(0.1% FA), Mobile Phase B: ACN; Flow rate: 60 mL/min; Gradient: 57% B to 65% B in 8 min, 65% B; Wave Length: 220 nm; RT1(min): 6.43. This provided 1-(2,2-difluoroethyl)-6-((3R,5S)-3-methyl-5-(((2-(trifluoromethyl)pyridin-3-yl)oxy)methyl)piperidin-1-yl)-1H-pyrazolo[3,4-b]pyrazine (47a, assumed, 7.3 mg, 3.9%) and 1-(2,2-difluoroethyl)-6-((3S,5S)-3-methyl-5-(((2-(trifluoromethyl)pyridin-3-yl)oxy)methyl)piperidin-1-yl)-1H-pyrazolo[3,4-b]pyrazine (47b, assumed, 44.1 mg, 22.7%) as white solids. 47a: ¹H NMR (400 MHZ, DMSO-d₆) δ 8.40 (s, 1H), 8.26-8.21 (m, 1H), 8.08 (s, 1H), 7.74 (d, J=8.4 Hz, 1H), 7.67-7.58 (m, 1H), 6.55-6.19 (m, 1H), 4.65-4.54 (m, 2H), 4.13 (d, J=7.1 Hz, 2H), 3.99-3.91 (m, 2H), 3.87-3.79 (m, 1H), 3.42-3.35 (m, 1H), 2.04 (d, J=30.2 Hz, 1H), 1.84-1.73 (m, 1H), 1.61-1.49 (m, 1H), 0.95 (d, J=6.7 Hz, 3H). MS m/z:456.9 [M+H]⁺. 47b: ¹H NMR (400 MHZ, DMSO-d₆) δ 8.47 (s, 1H), 8.30-8.25 (m, 1H), 8.13 (s, 1H), 7.82 (d, J=8.6 Hz, 1H), 7.74-7.67 (m, 1H), 6.57-6.24 (m, 1H), 4.94 (d, J=12.6 Hz, 1H), 4.73-4.61 (m, 2H), 4.52 (d, J=12.3 Hz, 1H), 4.27-4.20 (m, 1H), 4.07-3.99 (m, 1H), 2.73-2.54 (m, 2H), 2.17-2.07 (m, 1H), 1.90 (d, J=12.6 Hz, 1H), 1.77-1.66 (m, 1H), 1.13-1.02 (m, 1H), 0.98 (d, J=6.5 Hz, 3H). MS m/z: 456.9 [M+H]⁺.

### 1-(2,2-Difluoroethyl)-6-(4-methyl-3-(((2-(trifluoromethyl)pyridin-3-yl)oxy)methyl)piperidin-1-yl)-1H-pyrazolo[3,4-b]pyrazine (48)

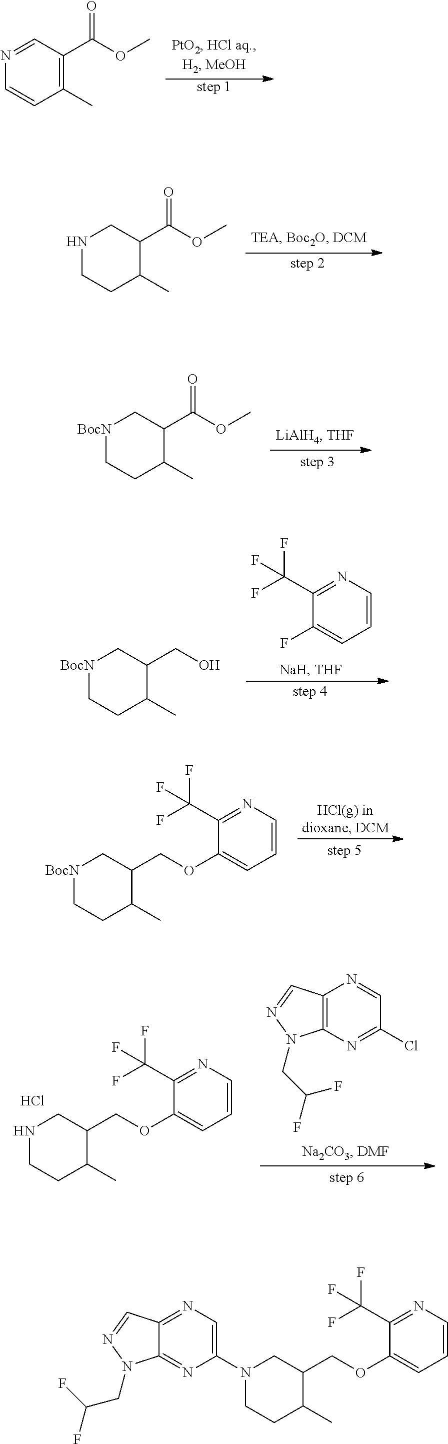

Step 1: methyl 4-methylpiperidine-3-carboxylate: To a stirred mixture of methyl 4-methylnicotinate (1 g, 6.615 mmol, 1 equiv) and PtO₂ (380 mg, 1.673 mmol, 0.25 equiv) in MeOH (5 mL) was added HCl (1 mL) at room temperature under air atmosphere. The resulting mixture was stirred for overnight at room temperature under hydrogen atmosphere. The resulting mixture was filtered, and the filter cake was washed with MeOH (3×10 mL). The filtrate was concentrated under reduced pressure, the crude product methyl 4-methylpiperidine-3-carboxylate (1 g, 96.15%) as a yellow oil. MS m/z: 158 [M+H]⁺.

Step 2: 1-(tert-butyl) 3-methyl 4-methylpiperidine-1,3-dicarboxylate: To a stirred mixture of methyl 4-methylpiperidine-3-carboxylate (1 g, 6.36 mmol, 1 equiv) in DCM (20 mL) was added TEA (2 g, 19.8 mmol, 3.11 equiv) at 0° C. and di-tert-butyl dicarbonate (1 g, 4.582 mmol, 0.72 equiv) at 0° C. under air atmosphere. The resulting mixture was stirred for 2 h at room temperature under air atmosphere. The resulting mixture was concentrated under reduced pressure. The residue was purified by silica gel column chromatography, eluted with PE/EA (1/1) to afford 1-(tert-butyl) 3-methyl 4-methylpiperidine-1,3-dicarboxylate (1.24 g, 75.8%) as a colorless oil. MS m/z: 258 [M+H]⁺.

Step 3: tert-butyl 3-(hydroxymethyl)-4-methylpiperidine-1-carboxylate: To a stirred mixture of 1-(tert-butyl) 3-methyl 4-methylpiperidine-1,3-dicarboxylate (1.2 g. 4.663 mmol, 1 equiv) in tetrahydrofuran (15 mL) was added LiAlH₄ (5.60 mL, 147.547 mmol, 31.64 equiv) dropwise at 0° C. under air atmosphere. The resulting mixture was stirred for 1 h at 0° C. under air atmosphere. The reaction was quenched by the addition of water (0.24 mL) at 0° C. Drop solution 10% NaOH (0.48 mL) into the mixture, last drop of water (0.72 mL). The resulting mixture was filtered, the filter cake was washed with EtOAc (3×10 mL), dried over anhydrous Na₂SO₄, filtered and concentrated to dryness under vacuum to give the crude product. The residue was purified by silica gel column chromatography, eluted with PE/EA (0-100% 20 min) to afford tert-butyl 3-(hydroxymethyl)-4-methylpiperidine-1-carboxylate (796 mg, 74.4%) as a colorless oil. MS m/z: 230 [M+H]⁺.

Step 4: tert-butyl 4-methyl-3-(((2-(trifluoromethyl)pyridin-3-yl)oxy)methyl)piperidine-1-carboxylate: To a stirred mixture of tert-butyl 3-(hydroxymethyl)-4-methylpiperidine-1-carboxylate (790 mg, 3.44 mmol, 1 equiv) in DMF (10 mL) was added NaH (60% in oil, 413 mg, 10.3 mmol, 3 equiv) in portions at 0° C. under air atmosphere. Stirred mixture at room temperature for 10 minutes. A solution of 3-fluoro-2-(trifluoromethyl)pyridine (569 mg, 3.44 mmol, 1 equiv) stirred for overnight at room temperature under air atmosphere. The reaction mixture was quenched with water (50 mL), extracted with EtOAc (3×50 mL). The combined organic layers were washed with brine (50 mL), dried over anhydrous Na₂SO₄. After filtration, the filtrate was concentrated to dryness under reduced pressure. The residue was purified by silica gel column chromatography, eluted with PE/EA (0-100% 20 min) to afford tert-butyl 4-methyl-3-(((2-(trifluoromethyl)pyridin-3-yl)oxy)methyl)piperidine-1-carboxylate (1.12 g, 86.8%) as a colorless oil. LCMS (ES, m/z): 375 [M+H]⁺.

Step 5: 3-((4-methylpiperidin-3-yl)methoxy)-2-(trifluoromethyl)pyridine hydrochloride: To a stirred mixture of tert-butyl 4-methyl-3-(((2-(trifluoromethyl)pyridin-3-yl)oxy)methyl)piperidine-1-carboxylate (1.1 g, 2.938 mmol, 1 equiv) in DCM (10 mL) was added HCl (gas) in 1,4-dioxane (10 mL) dropwise at 0° C. under air atmosphere. The resulting mixture was stirred for 1 h at 0° C. under air atmosphere. The resulting mixture was concentrated to dryness under vacuum. This provided 3-((4-methylpiperidin-3-yl)methoxy)-2-(trifluoromethyl)pyridine hydrochloride (946 mg, crude) as a white solid. MS m/z: 275 [M+H]⁺.

Step 6: 1-(2,2-difluoroethyl)-6-(4-methyl-3-(((2-(trifluoromethyl)pyridin-3-yl)oxy)methyl)pi-peridin-1-yl)-1H-pyrazolo[3,4-b]pyrazine: To a stirred mixture of 3-((4-methylpiperidin-3-yl)methoxy)-2-(trifluoromethyl)pyridine (162.29 mg, 0.522 mmol, 1.1 equiv) and Na₂CO₃ (150.97 mg, 1.425 mmol, 3 equiv) in DMF (1.5 mL) was added 6-chloro-1-(2,2-difluoroethyl)-1H-pyrazolo[3,4-b]pyrazine (100 mg, 0.475 mmol, 1 equiv) at room temperature under air atmosphere. The resulting mixture was stirred for 1.5 h at 100° C. under air atmosphere. The resulting mixture was diluted with water (20 mL) and extracted with EtOAc (3×15 mL). The combined organic layers were washed with water (2×30 mL) and brine (30 mL), dried over anhydrous Na₂SO₄. After filtration, the filtrate was concentrated to dryness under reduced pressure. The product was further purified by reversed phase Combi-flash chromatography with the following conditions (column, C18 gel; mobile phase, B phase: MeCN, A phase: water; 35% to 80% B gradient in 20 min; detector: UV 254/220 nm). The pure fraction was concentrated under vacuum to afford 1-(2,2-difluoroethyl)-6-(2-methyl-3-(((2-(trifluoromethyl)pyridin-3-yl)oxy)methyl)piperidin-1-yl)-1H-pyrazolo[3,4-b]pyrazine (80.6 mg, 33.5%) as a yellow solid. ¹H NMR (300 MHZ, DMSO-d₆) δ 8.43 (s, 1H), 8.32-8.24 (m, 1H), 8.13 (s, 1H), 7.87 (d, J=8.7 Hz, 1H), 7.72 (dd, J=8.7, 4.5 Hz, 1H), 6.41 (tt, J=54.9, 3.9 Hz, 1H), 4.85 (d, J=13.2 Hz, 1H), 4.75-4.60 (m, 2H), 4.52 (d, J=13.5 Hz, 1H), 4.44 (dd, J=9.9, 2.7 Hz, 1H), 4.08 (dd, J=9.6, 7.2 Hz, 1H), 3.12-3.01 (m, 1H), 2.95-2.82 (m, 1H), 1.86-1.64 (m, 3H), 1.41-1.22 (m, 1H), 1.03 (d, J=5.7 Hz, 3H). MS m/z: 457.2 [M+H]⁺.

### 1-(2,2-Difluoroethyl)-6-((3R,4R)-4-methyl-3-(((2-(trifluoromethyl)pyridin-3-yl)oxy)methyl)piperidin-1-yl)-1H-pyrazolo[3,4-b]pyrazine (48a); 1-(2,2-difluoroethyl)-6-((3S,4S)-4-methyl-3-(((2-(trifluoromethyl)pyridin-3-yl)oxy)methyl)piperidin-1-yl)-1H-pyrazolo[3,4-b]pyrazine (48b); 1-(2,2-difluoroethyl)-6-((3R,4S)-4-methyl-3-(((2-(trifluoromethyl)pyridin-3-yl)oxy)methyl)piperidin-1-yl)-1H-pyrazolo[3,4-b]pyrazine (48c); 1-(2,2-difluoroethyl)-6-((3S,4R)-4-methyl-3-(((2-(trifluoromethyl)pyridin-3-yl)oxy)methyl)piperidin-1-yl)-1H-pyrazolo[3,4-b]pyrazine(48d)

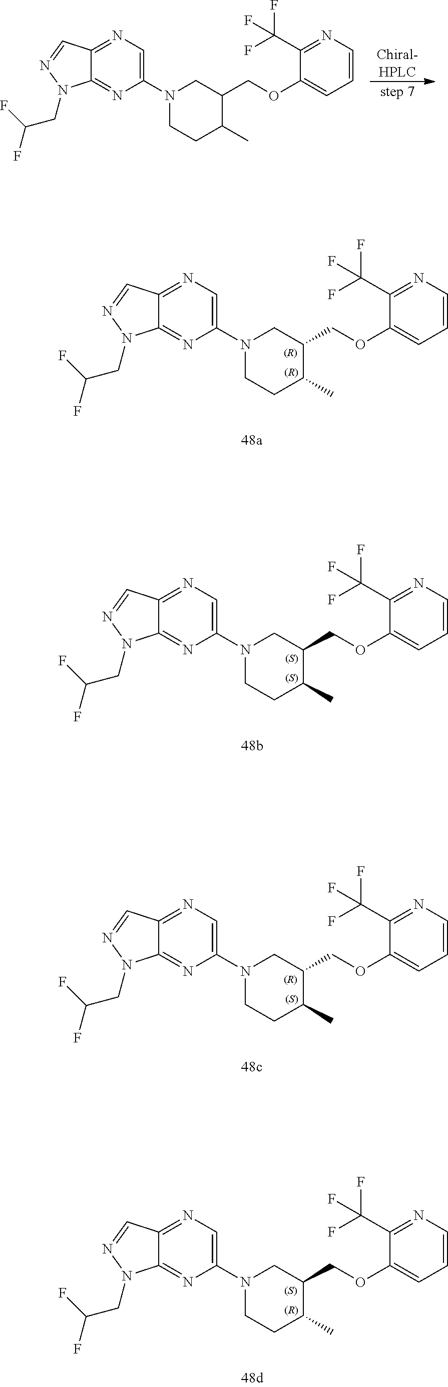

Compound 48 was further purified by prep. HPLC with the following conditions: Column: Xselect CSH F-Phenyl OBD column, 19*250 mm, 5 um; Mobile Phase A: Water(0.1% FA), Mobile Phase B: MeOH ----- Preparative; Flow rate: 25 mL/min; Gradient: 56% B to 72% B in 10 min, 72% B; Wave Length: 254/220 nm; RT1(min): 8.83/9.88. The resulting two racemates were further purified by chiral. HPLC with the following conditions: Column: CHIRALPAK IG-3, 4.6*50 mm, 3 um; Mobile Phase A: Hex(0.1% DEA):IPA=70:30; Flow rate: 1 mL/min; Gradient: 0% B to 0% B; Injection Volume: 5 μl mL. This provided 1-(2,2-difluoroethyl)-6-((3R,4R)-4-methyl-3-(((2-(trifluoromethyl)pyridin-3-yl)oxy)methyl)piperidin-1-yl)-1H-pyrazolo[3,4-b]pyrazine (48a, 17.1 mg, 21.2%), 1-(2,2-difluoroethyl)-6-((3S,4S)-4-methyl-3-(((2-(trifluoromethyl)pyridin-3-yl)oxy)methyl)piperidin-1-yl)-1H-pyrazolo[3,4-b]pyrazine(48b, 16.6 mg, 20.6%), 1-(2,2-difluoroethyl)-6-((3R,4S)-4-methyl-3-(((2-(trifluoromethyl)pyridin-3-yl)oxy)methyl)piperidin-1-yl)-1H-pyrazolo[3,4-b]pyrazine(48c, 20.2 mg, 25.1%) and 1-(2,2-difluoroethyl)-6-((3S,4R)-4-methyl-3-(((2-(trifluoromethyl)pyridin-3-yl)oxy)methyl)piperidin-1-yl)-1H-pyrazolo[3,4-b]pyrazine(48d, 18.3 mg, 22.7%) as white solids.

48a: ¹H NMR (400 MHZ, DMSO-d₆) δ 8.39 (d, J=28.6 Hz, 1H), 8.31-8.20 (m, 1H), 8.10 (d, J=18.7 Hz, 1H), 7.83 (dd, J=31.0, 8.7 Hz, 1H), 7.67 (d, J=37.2 Hz, 1H), 6.41 (t, J=55.0 Hz, 1H), 4.89-4.79 (m, 1H), 4.67 (t, J=15.0 Hz, 2H), 4.52 (d, J=14.4 Hz, 1H), 4.44 (d, J=9.7 Hz, 1H), 4.08 (s, 1H), 3.06 (s, 1H), 2.87 (d, J=12.0 Hz, 1H), 1.76 (q, J=14.5, 14.1 Hz. 3H), 1.32 (d, J=12.1 Hz, 1H), 1.09-0.99 (m, 3H). MS m/z:457.1 [M+H]⁺.

48b: ¹H NMR (400 MHZ, DMSO-d₆) δ 8.43 (s, 1H), 8.28 (d, J=4.5 Hz, 1H), 8.13 (s, 1H), 7.86 (d, J=8.5 Hz, 1H), 7.72 (dd, J=9.0, 4.6 Hz, 1H), 6.34 (d, J=55.1 Hz, 1H), 4.85 (d, J=13.6 Hz, 1H), 4.67 (t, J=15.2 Hz, 2H), 4.51 (d, J=13.1 Hz, 1H), 4.43 (d, J=9.4 Hz, 1H), 4.07 (d, J=8.7 Hz, 1H), 3.06 (s, 1H), 2.87 (d, J=12.1 Hz, 1H), 1.84-1.71 (m, 3H), 1.28 (d, J=35.8 Hz, 1H), 1.02 (d, J=5.6 Hz, 3H). MS m/z: 457.1 [M+H]⁺.

48c: ¹H NMR (400 MHZ, DMSO-d₆) δ 8.35 (s, 1H), 8.23 (d, J=4.4 Hz, 1H), 8.08 (s, 1H), 7.79 (d, J=8.6 Hz, 1H), 7.62 (dd, J=8.6, 4.5 Hz, 1H), 6.36 (t, J=4.0 Hz, 1H), 4.57 (d, J=14.7, 4.0 Hz, 2H), 4.20 (dd, J=9.7, 4.8 Hz, 1H), 4.09 (t, J=9.6 Hz, 1H), 3.99 (dd, J=13.5, 6.6 Hz, 2H), 3.80 (dd, J=13.4, 3.3 Hz, 1H), 3.59 (dd, J=11.1, 6.5 Hz, 1H), 2.26 (s, 1H), 2.14 (s, 1H), 1.70 (s, 1H), 1.56 (s, 1H), 1.06 (d, J=7.1 Hz, 3H). MS m/z: 457.1 [M+H]⁺.

48d: ¹H NMR (400 MHZ, DMSO-d₆) δ 8.36 (s, 1H), 8.23 (dd, J=4.5, 1.1 Hz, 1H), 8.08 (s, 1H), 7.79 (d, J=8.6 Hz, 1H), 7.62 (dd, J=8.6, 4.5 Hz, 1H), 6.48-6.23 (m, 1H), 4.57 (d, J=14.8, 4.0 Hz, 2H), 4.20 (dd, J=9.7, 4.9 Hz, 1H), 4.09 (t, J=9.6 Hz, 1H), 3.99 (dd, J=13.6, 6.3 Hz, 2H), 3.80 (dd, J=13.4, 3.5 Hz, 1H), 3.58 (td, J=8.6, 8.1, 4.2 Hz, 1H), 2.26 (s, 1H), 2.14 (d, J=4.8 Hz, 1H), 1.77-1.66 (m, 1H), 1.60-1.50 (m, 1H), 1.06 (d, J=7.1 Hz, 3H). MS m/z: 457.1 [M+H]⁺.

### (5-methyl-6-phenyl-5H-pyrrolo[2,3-b]pyrazin-3-yl)(3-((o-tolyloxy)methyl)piperidin-1-yl)methanone (49)

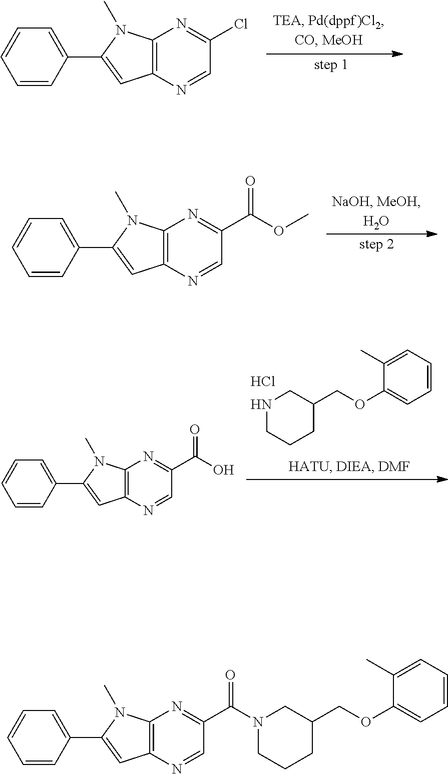 [SMILES: Cn1c(-c2ccccc2)cc2ncc(Cl)nc21 ; COC(=O)c1cnc2cc(-c3ccccc3)n(C)c2n1 ; Cn1c(-c2ccccc2)cc2ncc(C(=O)O)nc21 ; Cl ; Cc1ccccc1OCC1CCCNC1 ; Cc1ccccc1OCC1CCCN(C(=O)c2cnc3cc(-c4ccccc4)n(C)c3n2)C1]

Step 1: methyl 5-methyl-6-phenyl-5H-pyrrolo[2,3-b]pyrazine-3-carboxylate: To a stirred solution of 3-chloro-5-methyl-6-phenyl-5H-pyrrolo[2,3-b]pyrazine (300 mg, 1.23 mmol, 1.00 equiv) and TEA (373 mg, 3.69 mmol, 3 equiv) in MeOH (5 mL) was added Pd(dppf)Cl₂ (90.0 mg, 0.123 mmol, 0.1 equiv) at room temperature. The resulting mixture was stirred for 16 h at 100° C. under CO atmosphere in 50 atm. The reaction mixture was diluted by EtOAc (50 mL), washed by water (2×40 mL) and brine (1×40 mL), dried over anhydrous sodium sulfate. After filtration, the filtrate was concentrated. The residue was purified by silica gel column chromatography, eluted with EtOAc/PE=1/2, to afford methyl 5-methyl-6-phenyl-5H-pyrrolo[2,3-b]pyrazine-3-carboxylate (300 mg, 91.1%) as a brown solid. MS m/z: 268 [M+H]⁺.

Step 2: 5-methyl-6-phenyl-5H-pyrrolo[2,3-b]pyrazine-3-carboxylic acid: To a stirred solution of 5-methyl-6-phenyl-5H-pyrrolo[2,3-b]pyrazine-3-carboxylate (300 mg, 1.12 mmol, 1.00 equiv) and NaOH (179 mg, 4.48 mmol, 4 equiv) in MeOH (2 mL)/H₂O (2 mL) at room temperature. The resulting mixture was stirred for 2 hours at 50° C. Desired product could be detected by LCMS. The mixture was acidified to pH 5 with HCl (1 mol/L). The resulting mixture was extracted with EtOAc (3×10 mL). The combined organic layers were washed with water (3×15 mL), dried over anhydrous Na₂SO₄. After filtration, the filtrate was concentrated under reduced pressure. This provided 5-methyl-6-phenyl-5H-pyrrolo[2,3-b]pyrazine-3-carboxylic acid (260 mg, 95.6%) as a brown solid. MS m/z: 254 [M+H]⁺.

Step 3: (5-methyl-6-phenyl-5H-pyrrolo[2,3-b]pyrazin-3-yl)(3-((o-tolyloxy)methyl)piperidin-1-yl)methanone: To a stirred solution of 5-methyl-6-phenylpyrrolo[2,3-b]pyrazine-3-carboxylic acid (100 mg, 0.395 mmol, 1.00 equiv) and HATU (165 mg, 0.435 mmol, 1.1 equiv) in DMF (1.5 mL) were added DIEA (204 mg, 1.580 mmol, 4 equiv) and 3-(2-methylphenoxymethyl)piperidine hydrochloride (105 mg, 0.435 mmol, 1.1 equiv) dropwise at 0° C. The resulting mixture was stirred for 3 hours at 0° C. Desired product could be detected by LCMS. The residue was purified by reverse flash chromatography with the following conditions: column, C18 silica gel; mobile phase, MeCN in water, 0% to 100% gradient in 30 min; detector, UV 254 nm. This provided (5-methyl-6-phenyl-5H-pyrrolo[2,3-b]pyrazin-3-yl)(3-((o-tolyloxy)methyl)piperidin-1-yl) methanone (60 mg, 33.84%) as a yellow solid. ¹H NMR (400 MHZ, DMSO-d₆) δ 8.64-8.56 (m, 1H), 7.81-7.69 (m, 2H), 7.66-7.53 (m, 3H), 7.20-6.94 (m, 2H), 6.92-6.92 (m, 1H), 6.91-6.69 (m, 2H), 4.65-4.31 (m, 1H), 4.05-3.87 (m, 2H), 3.85 (s, 1H), 3.74 (s, 2H), 3.70-3.65 (m, 1H), 3.24-2.87 (m, 2H), 2.22 (s, 1H), 2.15-2.10 (m, 1H), 1.93-1.90 (m, 1H), 1.85-1.81 (m, 1H), 1.71-1.61 (m, 1H), 1.49 (s, 3H). MS m/z: 441.3 [M+H]⁺.

### (5-Methyl-6-phenyl-5H-pyrrolo[2,3-b]pyrazin-3-yl)(3-(((2-(trifluoromethyl)pyridin-3-yl)oxy)methyl)piperidin-1-yl)methanone (50)

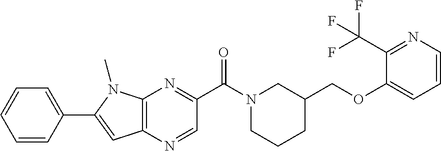 [SMILES: Cn1c(-c2ccccc2)cc2ncc(C(=O)N3CCCC(COc4cccnc4C(F)(F)F)C3)nc21]

Followed General Procedure E using 5-methyl-6-phenyl-5H-pyrrolo[2,3-b]pyrazine-3-carboxylic acid (100 mg, 0.395 mmol, 1.00 equiv) and 3-(piperidin-3-ylmethoxy)-2-(trifluoromethyl)pyridine hydrochloride (140 mg, 0.474 mmol, 1.2 equiv) to afford (5-methyl-6-phenyl-5H-pyrrolo[2,3-b]pyrazin-3-yl)(3-(((2-(trifluoromethyl)pyridin-3-yl)oxy) methyl)piperidin-1-yl)methanone (60 mg, 29.3%) as a yellow solid. ¹H NMR (400 MHz, DMSO-d₆) δ 8.55-8.52 (m, 1H), 8.29-8.16 (m, 1H), 7.90-7.75 (m, 1H), 7.75-7.67 (m, 2H), 7.65-7.47 (m, 4H), 6.93-6.89 (m, 1H), 4.66-4.15 (m, 2H), 4.13-4.09 (m, 1H), 3.99-3.59 (m, 4H), 3.19-2.86 (m, 2H), 2.13-2.08 (m, 1H), 2.00-1.35 (m, 4H). MS m/z: 496.2 [M+H]⁺.

### (5-(Difluoro(phenyl)methyl)-1,3,4-oxadiazol-2-yl)(3-(phenoxymethyl)piperidin-1-yl)methanone (51)

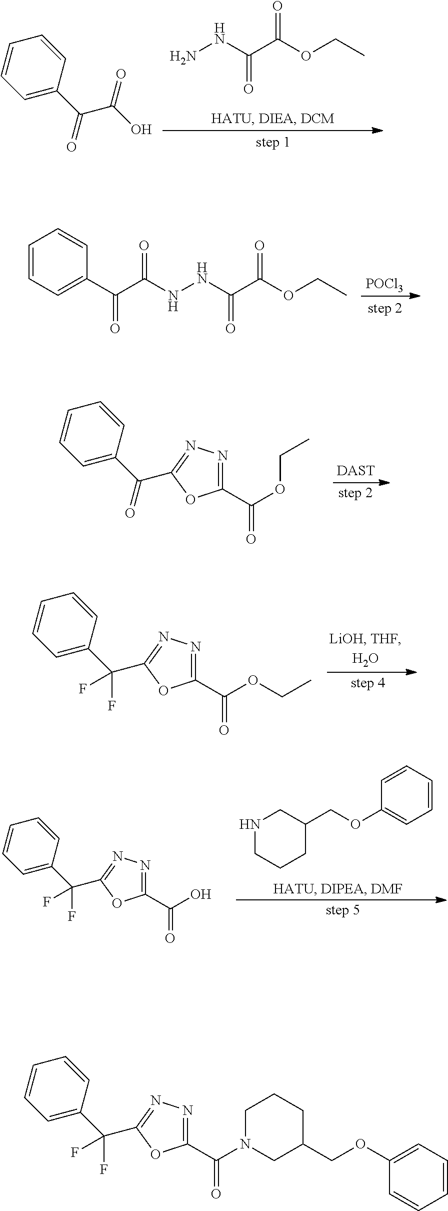

Step 1: ethyl 2-oxo-2-(2-(2-oxo-2-phenylacetyl)hydrazineyl)acetate: A mixture of benzoylformic acid (700 mg, 4.66 mmol, 1 equiv), ethyl (hydrazinecarbonyl)formate (677 mg, 5.13 mmol, 1.1 equiv), HATU (2.66 g, 6.99 mmol, 1.5 equiv) and DIPEA (1.21 g, 9.33 mmol, 2 equiv) in DCM (20 mL) was stirred for 3 h at room temperature. The resulting mixture was washed with water (3×10 mL). The resulting mixture was concentrated under vacuum. The residue was purified by reverse flash chromatography with the following conditions: column, C18 silica gel; mobile phase, MeCN in water, 10% to 100% gradient in 30 min; detector, UV 254 nm. This provided ethyl 2-oxo-2-(2-(2-oxo-2-phenylacetyl)hydrazineyl)acetate (750 mg, 60.9%) as a white solid. MS m/z: 265[M+H]⁺.

Step 2. ethyl 5-benzoyl-1,3,4-oxadiazole-2-carboxylate: A solution of ethyl 2-oxo-2-(2-(2-oxo-2-phenylacetyl)hydrazineyl)acetate (750 mg, 2.84 mmol, 1.00 equiv) in phosphorus oxychloride (6 mL) was stirred for 8 h at 100° C. The reaction was quenched by the addition of sat. NaHCO₃(aq.) (30 mL) at 0° C. The resulting mixture was extracted with EtOAc (3×20 mL). The combined organic layers were washed with water (2×15 mL), dried over anhydrous Na₂SO₄. After filtration, the filtrate was concentrated under reduced pressure. The residue was purified by silica gel column chromatography, eluted with PE/EA (5:1) to afford ethyl 5-benzoyl-1,3,4-oxadiazole-2-carboxylate (300 mg, 42.9%) as a yellow solid. MS m/z: 247 [M+H]⁺.

Step 3: ethyl 5-(difluoro(phenyl)methyl)-1,3,4-oxadiazole-2-carboxylate: A solution of ethyl 5-benzoyl-1,3,4-oxadiazole-2-carboxylate (300 mg, 1.22 mmol, 1.00 equiv) and DAST (1.96 g, 12.2 mmol, 10 equiv) in DCM (5 mL) was stirred for overnight at 50° C. The resulting mixture was concentrated under vacuum. The residue was purified by silica gel column chromatography, eluted with PE/EA (5:1) to afford ethyl 5-[difluoro(phenyl)methyl]-1,3,4-oxadiazole-2-carboxylate (150 mg, 45.9%) as a yellow solid. MS m/z: 269 [M+H]⁺.

Step 4: 5-(difluoro(phenyl)methyl)-1,3,4-oxadiazole-2-carboxylic acid: A solution of ethyl 5-(difluoro(phenyl)methyl)-1,3,4-oxadiazole-2-carboxylate (150 mg, 0.560 mmol, 1.00 equiv) and LiOH (16.1 mg, 0.671 mmol, 1.2 equiv) in THF (0.5 mL)/MeOH (0.5 mL)/H₂O (0.5 mL) was stirred for overnight at room temperature. The resulting mixture was concentrated under vacuum. The crude product (140 mg) was used in the next step directly without further purification. MS m/z: 241 [M+H]⁺.

Step 5: (5-(difluoro(phenyl)methyl)-1,3,4-oxadiazol-2-yl)(3-(phenoxymethyl)piperidin-1-yl)methanone: Followed General Procedure E using 5-(difluoro(phenyl)methyl)-1,3,4-oxadiazole-2-carboxylic acid (50 mg, 0.208 mmol, 1.00 equiv) and 3-(phenoxymethyl)piperidine (43.8 mg, 0.229 mmol, 1.1 equiv) to afford (5-(difluoro(phenyl)methyl)-1,3,4-oxadiazol-2-yl)(3-(phenoxymethyl)piperidin-1-yl)methanone (20 mg, 23.2%) as a white solid. ¹H NMR (300 MHz, DMSO-d₆) δ 7.77-7.57 (m, 5H), 7.34-7.22 (m, 2H), 7.00-6.84 (m, 3H), 4.55-4.18 (m, 2H), 3.99-3.79 (m, 2H), 3.42-3.33 (m, 1H), 3.15-2.91 (m, 1H), 2.17-1.98 (s, 1H), 1.96-1.71 (m, 2H), 1.67-1.37 (m, 2H). MS m/z: 413.90 [M+H]⁺.

### (5-methyl-6-phenyl-5H-pyrrolo[2,3-b]pyrazin-2-yl)(3-((o-tolyloxy)methyl)piperidin-1-yl)methanone (52)

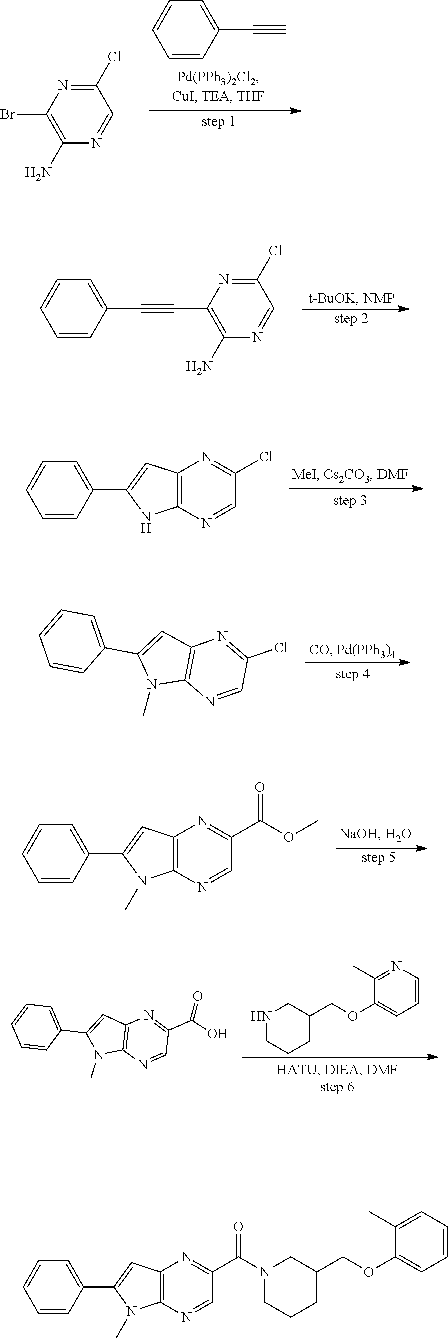

Step 1: 5-chloro-3-(phenylethynyl)pyrazin-2-amine: To the solution of 3-bromo-5-chloropyrazin-2-amine (600 mg, 2.87 mmol, 1.00 equiv) and ethynylbenzene (352 mg, 3.45 mmol, 1.2 equiv) in THF (6 mL) were added CuI (54.8 mg, 0.288 mmol, 0.1 equiv) and TEA (873 mg, 8.63 mmol, 3 equiv) and Pd(PPh₃)₂Cl₂ (202 mg, 0.288 mmol, 0.1 equiv) under N₂ atmosphere. The result mixture was heated to 80° C. and stirred 2 h. Desired product could be detected by LCMS. The reaction mixture was diluted by EtOAc (20 mL), washed by water (2×20 mL) and brine (1×30 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 90% gradient in 10 min; detector, UV 254 nm. This provided 5-chloro-3-(2-phenylethynyl)pyrazin-2-amine (400 mg, 60.5%) as a brown solid. MS m/z: 230 [M+H]⁺.

Step 2: 2-chloro-6-phenyl-5H-pyrrolo[2,3-b]pyrazine: To the solution of 5-chloro-3-(2-phenylethynyl)pyrazin-2-amine (400 mg, 1.74 mmol, 1.00 equiv) in NMP (5 mL) were added t-BuOK (977 mg, 8.71 mmol, 5 equiv) under N₂ atmosphere. The result mixture was heated to 80° C. and stirred 2 h. Desired product could be detected by LCMS. The reaction mixture was diluted by EtOAc (20 mL), washed by water (2×20 mL) and brine (1×30 mL), dried over anhydrous Na₂SO₄. After filtration, the filtrate was concentrated under reduced pressure. The residue was purified by reverse flash chromatography with the following conditions: column, silica gel; mobile phase, MeCN in water, 10% to 90% gradient in 10 min; detector, UV 254 nm. This provided 2-chloro-6-phenyl-5H-pyrrolo[2,3-b]pyrazine (350 mg, 87.5%) as a brown yellow solid. MS m/z: 230 [M+H]⁺.

Step 3: 2-chloro-5-methyl-6-phenyl-5H-pyrrolo[2,3-b]pyrazine: To the solution of 2-chloro-6-phenyl-5H-pyrrolo[2,3-b]pyrazine (400 mg, 1.74 mmol, 1.00 equiv) and MeI (370 mg, 2.61 mmol, 1.5 equiv) and Cs₂CO₃ (1134 mg, 3.48 mmol, 2 equiv) in DMF (4 mL) under N₂ atmosphere. The reaction lasted one night at room temperature. Desired product could be detected by LCMS. The resulting mixture was diluted with EtOAc (50 mL). The combined organic layers were washed with water (3×50 mL), dried over anhydrous Na₂SO₄. After filtration, the filtrate was concentrated under reduced pressure. The resulting mixture was concentrated under reduced pressure. The residue was purified by reverse flash chromatography with the following conditions: column, C18 silica gel; mobile phase, MeCN in water, 10% to 90% gradient in 10 min; detector, UV 254 nm. This provided 2-chloro-5-methyl-6-phenyl-5H-pyrrolo[2,3-b]pyrazine (300 mg, 70.6%) as a yellow green solid. MS m/z: 244 [M+H]⁺.

Step 4: methyl 5-methyl-6-phenyl-5H-pyrrolo[2,3-b]pyrazine-2-carboxylate: To a solution of 2-chloro-5-methyl-6-phenyl-5H-pyrrolo[2,3-b]pyrazine in MeOH (5 mL) was added Pd(PPh₃)₂Cl₂ (67.0 mg, 0.09 mmol, 0.1 equiv) and TEA (290 mg, 2.86 mmol, 3 equiv) in a pressure tank. The mixture was purged with nitrogen for 1 hour and then was pressurized to 50 atm with carbon monoxide at 100° C. for one night. Desired product could be detected by LCMS. The reaction mixture was cooled to room temperature and filtered to remove insoluble solids. The resulting mixture was concentrated under reduced pressure. The residue was purified by reverse flash chromatography with the following conditions: column, C18 silica gel; mobile phase, MeCN in water, 10% to 90% gradient in 10 min; detector, UV 254 nm. This provided methyl 6-(4-fluorophenyl)-5-methylpyrrolo[2,3-b]pyrazine-2-carboxylate (220 mg, 80.7%) as a brown solid. MS m/z: 268 [M+H]⁺.

Step 5: 5-methyl-6-phenyl-5H-pyrrolo[2,3-b]pyrazine-2-carboxylic acid: To the solution of methyl 6-(4-fluorophenyl)-5-methylpyrrolo[2,3-b]pyrazine-2-carboxylate (180 mg, 0.673 mmol, 1 equiv) and NaOH (53.8 mg, 1.34 mmol, 2 equiv) in H₂O (2 mL) under N₂ atmosphere. The result mixture was heated to 50° C. and stirred 2 h. Desired product could be detected by LCMS. The residue was acidified to pH 3 with HCl(0.5 mL, 1.0 mmol). The resulting mixture was extracted with EtOAc (3×20 mL). The combined organic layers were washed with water (30 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 90% gradient in 10 min; detector, UV 254 nm. This provided 5-methyl-6-phenylpyrrolo[2,3-b]pyrazine-2-carboxylic acid (120 mg, 70.3%) as a white solid. MS m/z: 254 [M+H]⁺.

Step 6: (5-methyl-6-phenyl-5H-pyrrolo[2,3-b]pyrazin-2-yl)(3-((o-tolyloxy)methyl)piperidin-1-yl)methanone: Followed General Procedure E using 5-methyl-6-phenylpyrrolo[2,3-b]pyrazine-2-carboxylic acid (100 mg, 0.395 mmol, 1 equiv) and 2-methyl-3-(piperidin-3-ylmethoxy)pyridine (97.7 mg, 0.474 mmol, 1.2 equiv) to afford (5-methyl-6-phenyl-5H-pyrrolo[2,3-b]pyrazin-2-yl)(3-((o-tolyloxy)methyl)piperidin-1-yl)methanone (40 mg, 23.0%) as a white solid. ¹H NMR (300 MHz, DMSO-d₆) δ 8.55-8.38 (m, 1H), 7.79-7.70 (m, 2H), 7.66-7.53 (m, 3H), 7.22-6.88 (m, 3H), 6.88-6.79 (m, 1H), 6.79-6.69 (m, 1H), 4.68-4.24 (m, 1H), 3.99-3.62 (m, 6H), 3.21-2.85 (m, 2H), 2.22 (s, 1H), 2.07 (s, 1H), 1.96-1.70 (m, 2H), 1.67-1.38 (m, 4H). MS m/z: 441.1 [M+H]⁺.

### (1-Methyl-2-phenyl-1H-imidazo[4,5-b]pyrazin-6-yl)(3-((o-tolyloxy)methyl)piperidin-1-yl)methanone (53)

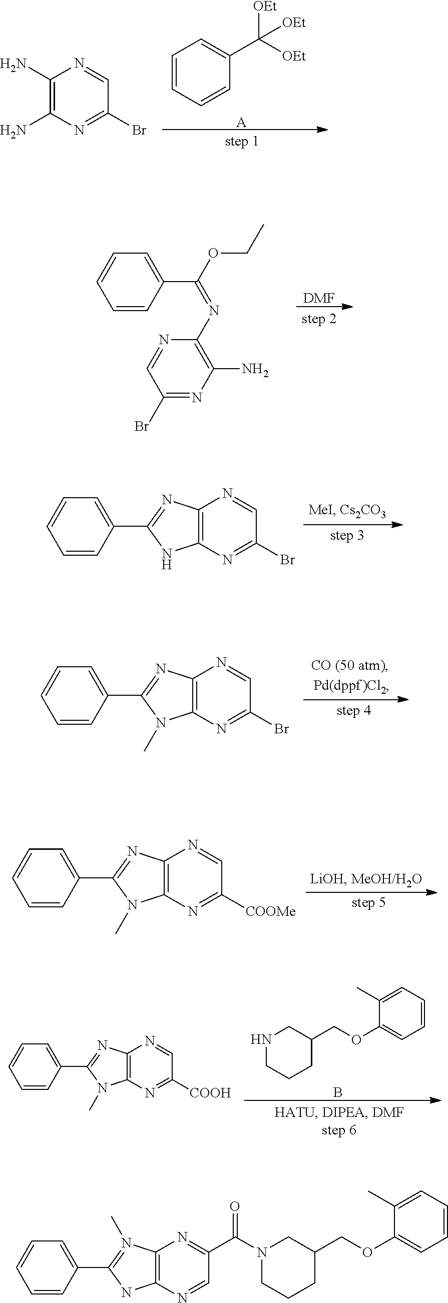

Step 1: ethyl (E)-N-(3-amino-5-bromopyrazin-2-yl)benzimidate: A solution of 5-bromopyrazine-2,3-diamine (3 g, 15.9 mmol, 1 equiv) in (triethoxymethyl)benzene (40 mL) was stirred for 2 days at 130° C. The residue was purified by silica gel column chromatography, eluted with PE/EA (5:1) to afford ethyl (E)-N-(3-amino-5-bromopyrazin-2-yl)benzimidate (700 mg, 13.7%) as a yellow solid. MS m/z: 321 [M+H]⁺.

Step 2: 6-bromo-2-phenyl-1H-imidazo[4,5-b]pyrazine: A solution of ethyl (E)-N-(3-amino-5-bromopyrazin-2-yl)benzimidate (700 mg) in DMF (5 mL) was stirred for overnight at 110° C. The resulting mixture was extracted with EtOAc (40 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford 6-bromo-2-phenyl-1H-imidazo[4,5-b]pyrazine (400 mg). MS m/z: 275 [M+H]⁺.

Step 3: 6-bromo-1-methyl-2-phenyl-1H-imidazo[4,5-b]pyrazine: A solution of 6-bromo-2-phenyl-1H-imidazo[4,5-b]pyrazine (400 mg, 1.44 mmol, 1.00 equiv), CH₃I (247 mg, 1.74 mmol, 1.2 equiv) and Cs₂CO₃ (947 mg, 2.91 mmol, 2 equiv) in DMF (5 mL) was stirred for overnight at room temperature. The resulting mixture was extracted with EtOAc (30 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:1) to afford 6-bromo-1-methyl-2-phenyl-1H-imidazo[4,5-b]pyrazine (280 mg, 66.6%) as a yellow solid. MS m/z: 289 [M+H]⁺.

Step 4: methyl 1-methyl-2-phenyl-1H-imidazo[4,5-b]pyrazine-6-carboxylate: A solution of 6-bromo-1-methyl-2-phenyl-1H-imidazo[4,5-b]pyrazine (280 mg, 0.968 mmol, 1.00 equiv), Et₃N (294 mg, 2.90 mmol, 3 equiv) and Pd(dppf)Cl₂ (70.8 mg, 0.097 mmol, 0.1 equiv) in MeOH (10 mL) was stirred for overnight at 100° C. under carbon monoxide atmosphere. The resulting mixture was concentrated under vacuum. The residue was purified by silica gel column chromatography, eluted with PE/EA (1:2) to afford methyl 1-methyl-2-phenyl-1H-imidazo[4,5-b]pyrazine-6-carboxylate (200 mg, 77.0%) as a yellow solid. MS m/z: 269 [M+H]⁺.

Step 5: 1-methyl-2-phenyl-1H-imidazo[4,5-b]pyrazine-6-carboxylic acid: A solution of methyl 1-methyl-2-phenyl-1H-imidazo[4,5-b]pyrazine-6-carboxylate (200 mg, 0.746 mmol, 1 equiv) and LiOH (21.4 mg, 0.895 mmol, 1.2 equiv) in THF (2 mL)/H₂O (2 mL)/MeOH (2 mL) was stirred for overnight at room temperature. The resulting mixture was concentrated under vacuum. The crude product/resulting mixture was used in the next step directly without further purification. MS m/z: 255 [M+H]⁺.

Step 6: (1-methyl-2-phenyl-1H-imidazo[4,5-b]pyrazin-6-yl)(3-((o-tolyloxy)methyl)piperidin-1-yl)methanone: Followed General Procedure E using 1-methyl-2-phenyl-1H-imidazo[4,5-b]pyrazine-6-carboxylic acid (150 mg, 0.59 mmol, 1 equiv) and 3-((o-tolyloxy)methyl)piperidine (133 mg, 0.65 mmol, 1.1 equiv) to afford (1-methyl-2-phenyl-1H-imidazo[4,5-b]pyrazin-6-yl)(3-((o-tolyloxy)methyl) piperidin-1-yl)methanone (15 mg, 5.70%) as a white solid. ¹H NMR (400 MHZ, DMSO-d₆) δ 8.68 (d, J=24.7 Hz, 1H), 8.05-7.97 (m, 2H), 7.70-7.62 (m, 3H), 7.21-6.64 (m, 4H), 4.69-4.22 (m, 1H), 4.04-3.64 (m, 6H), 3.21-2.87 (m, 2H), 2.22 (s, 1H), 2.17-2.04 (m, 1H), 2.00-1.87 (m, 1H), 1.86-1.67 (m, 1H), 1.65-1.35 (m, 4H). MS m/z: 442.2 [M+H]⁺.

### (1-Methyl-2-phenyl-1H-imidazo[4,5-b]pyrazin-5-yl)(3-((o-tolyloxy)methyl)piperidin-1-yl)methanone (54)

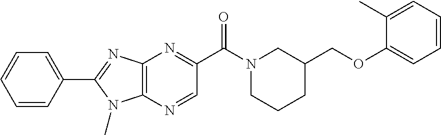 [SMILES: Cc1ccccc1OCC1CCCN(C(=O)c2cnc3c(n2)nc(-c2ccccc2)n3C)C1]

Followed General Procedure E using 1-methyl-2-phenyl-1H-imidazo[4,5-b]pyrazine-5-carboxylic acid (50 mg, 0.197 mmol, 1.00 equiv) and 3-((o-tolyloxy)methyl)piperidine (44.4 mg, 0.217 mmol, 1.1 equiv) to afford (1-methyl-2-phenyl-1H-imidazo[4,5-b]pyrazin-5-yl)(3-((o-tolyloxy)methyl)piperidin-1-yl)methanone (15 mg, 17.1%) as a white solid. ¹H NMR (400 MHZ, DMSO-d₆) δ 8.60 (d, J=37.4 Hz, 1H), 8.07-8.00 (m, 2H), 7.73-7.62 (m, 3H), 7.19-6.69 (m, 4H), 4.70-4.24 (m, 1H), 4.02-3.58 (m, 6H), 3.23-2.88 (m, 2H), 2.22 (s, 1H), 2.15-2.00 (m, 1H), 1.98-1.83 (m, 1H), 1.85-1.65 (m, 1H), 1.63-1.42 (s, 4H). MS m/z: 442.20 [M+H]⁺.

### (6-(4-Fluorophenyl)-5-methyl-5H-pyrrolo[2,3-b]pyrazin-2-yl)(3-((2-(trifluoromethyl) phenoxy)methyl)piperidin-1-yl)methanone (55)

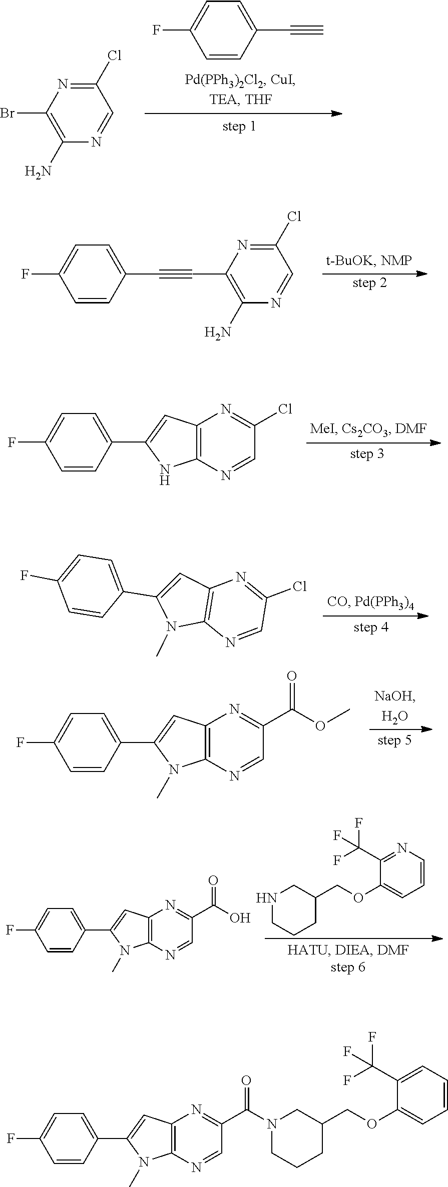

Step 1: 5-chloro-3-((4-fluorophenyl)ethynyl)pyrazin-2-amine: To a solution of 3-bromo-5-chloropyrazin-2-amine (600 mg, 2.87 mmol, 1.00 equiv) and 1-ethynyl-4-fluorobenzene (414 mg, 3.45 mmol, 1.2 equiv) in THF (6.5 mL) were added CuI (54.8 mg, 0.288 mmol, 0.1 equiv) and TEA (873 mg, 8.63 mmol, 3 equiv) and Pd(PPh₃)₂Cl₂ (202 mg, 0.288 mmol, 0.1 equiv) under N₂ atmosphere. The result mixture was heated to 80° C. and stirred 2 h. Desired product could be detected by LCMS. The reaction mixture was diluted by EtOAc (20 mL), washed by water (2×20 mL) and brine (1×30 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 50% gradient in 10 min; detector, UV 254 nm. This provided 5-chloro-3-((4-fluorophenyl)ethynyl)pyrazin-2-amine (500 mg, 70.1%) as a brown solid. MS m/z: 248 [M+H]⁺.

Step 2: 2-chloro-6-(4-fluorophenyl)-5H-pyrrolo[2,3-b]pyrazine: To the solution of 5-chloro-3-((4-fluorophenyl)ethynyl)pyrazin-2-amine (500 mg, 2.01 mmol, 1.00 equiv) in NMP (6 mL) were added t-BuOK (453 mg, 4.03 mmol, 2 equiv) under N₂ atmosphere. The resulting mixture was heated to 80° C. and stirred 2 h. Desired product could be detected by LCMS. The reaction mixture was diluted by EtOAc (20 mL), washed by water (2×20 mL) and brine (1×30 mL), dried over anhydrous Na₂SO₄. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm. This provided 2-chloro-6-(4-fluorophenyl)-5H-pyrrolo[2,3-b]pyrazine (350 mg, 70.0%) as a yellow solid. MS m/z: 248 [M+H]⁺.

Step 3: 2-chloro-6-(4-fluorophenyl)-5-methyl-5H-pyrrolo[2,3-b]pyrazine: To the solution of 2-chloro-6-(4-fluorophenyl)-5H-pyrrolo[2,3-b]pyrazine (350 mg, 1.41 mmol, 1.00 equiv) and MeI (300 mg, 2.12 mmol, 1.5 equiv) and Cs₂CO₃ (1381 mg, 4.24 mmol, 3 equiv) in DMF (6 mL) under N₂ atmosphere. The reaction lasted one night at room temperature. Desired product could be detected by LCMS. The resulting mixture was diluted with water (50 mL). The combined organic layers were extracted with EtOAc (3×50 mL), dried over anhydrous Na₂SO₄. After filtration, the filtrate was concentrated under reduced pressure. The resulting mixture was concentrated under reduced pressure. The residue was purified by reverse flash chromatography with the following conditions: column, C18 silica gel; mobile phase, MeCN in water, 10% to 50% gradient in 10 min; detector, UV 254 nm. This provided 2-chloro-6-(4-fluorophenyl)-5-methyl-5H-pyrrolo[2,3-b]pyrazine (250 mg, 77.2%) as a yellow solid. MS m/z: 262 [M+H]⁺.

Step 4: methyl 6-(4-fluorophenyl)-5-methyl-5H-pyrrolo[2,3-b]pyrazine-2-carboxylate: To a solution of 2-chloro-6-(4-fluorophenyl)-5-methyl-5H-pyrrolo[2,3-b]pyrazine (250 mg, 0.95 mmol, 1.00 equiv) in MeOH (5 mL) was added Pd(PPh₃)₂Cl₂ (67.0 mg, 0.096 mmol, 0.1 equiv) and TEA (290 mg, 2.86 mmol, 3 equiv) in a pressure tank. The mixture was purged with nitrogen for 1 hour and then was pressurized to 50 atm with carbon monoxide at 100° C. for one night. Desired product could be detected by LCMS. The reaction mixture was cooled to room temperature and filtered to remove insoluble solids. The resulting mixture was concentrated under reduced pressure. The residue was purified by reverse flash chromatography with the following conditions: column, C18 silica gel; mobile phase, MeCN in water, 10% to 50% gradient in 10 min; detector, UV 254 nm. This provided methyl 6-(4-fluorophenyl)-5-methyl-5H-pyrrolo[2,3-b]pyrazine-2-carboxylate (220 mg, 80.7%) as a brown solid. MS m/z: 286 [M+H]⁺.

Step 5: 6-(4-fluorophenyl)-5-methyl-5H-pyrrolo[2,3-b]pyrazine-2-carboxylic acid: To the solution of methyl 6-(4-fluorophenyl)-5-methylpyrrolo[2,3-b]pyrazine-2-carboxylate (220 mg, 0.769 mmol, 1 equiv) and NaOH (61.5 mg, 1.53 mmol, 2 equiv) in H₂O (2 mL) under N₂ atmosphere. The result mixture was heated to 50° C. and stirred 2 h. Desired product could be detected by LCMS. The residue was acidified to pH 3 with HCl (0.5 mL, 1.0 mmol). The resulting mixture was extracted with EtOAc (3×15 mL). The combined organic layers were washed with water (3×20 mL), dried over anhydrous Na₂SO₄. After filtration, the filtrate was concentrated under reduced pressure. The resulting mixture was concentrated under reduced pressure. The residue was purified by reverse flash chromatography with the following conditions: column, C18 silica gel; mobile phase, MeCN in water, 10% to 90% gradient in 10 min; detector, UV 254 nm. This provided 5-methyl-6-phenylpyrrolo[2,3-b]pyrazine-2-carboxylic acid (120 mg, 51.3%) as a white solid. MS m/z: 272 [M+H]⁺.

Step 6: (6-(4-fluorophenyl)-5-methyl-5H-pyrrolo[2,3-b]pyrazin-2-yl)(3-((2-(trifluoromethyl)phenoxy)methyl)piperidin-1-yl)methanone: Followed General Procedure E using 5-methyl-6-phenylpyrrolo5-methyl-6-phenylpyrrolo[2,3-b]pyrazine-2-carboxylic acid (60 mg, 0.221 mmol, 1 equiv) and 3-(piperidin-3-ylmethoxy)-2-(trifluoromethyl)pyridine (69.0 mg, 0.265 mmol, 1.2 equiv) to afford (6-(4-fluorophenyl)-5-methyl-5H-pyrrolo[2,3-b]pyrazin-2-yl)(3-((2-(trifluoromethyl)phenoxy)methyl)piperidin-1-yl)methanone (20.1 mg, 17.70%) as a yellow solid. ¹H NMR (300 MHZ, DMSO-d₆) δ 8.54-8.32 (m, 1H), 8.31-8.14 (m, 1H), 7.90-7.59 (m, 4H), 7.54-7.39 (m, 2H), 6.93-6.55 (m, 1H), 4.65-3.90 (m, 3H), 3.88-3.74 (m, 4H), 3.24-2.83 (m, 2H), 2.06 (s, 1H), 1.96-1.66 (m, 2H), 1.65-1.38 (m, 2H). MS m/z: 514.1 [M+H]⁺.

### (1-Methyl-2-phenyl-1H-imidazo[4,5-b]pyrazin-6-yl)(3-(((2-(trifluoromethyl)pyridin-3-yl)oxy)methyl)piperidin-1-yl)methanone (56)

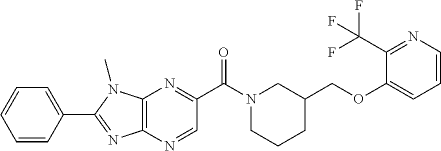 [SMILES: Cn1c(-c2ccccc2)nc2ncc(C(=O)N3CCCC(COc4cccnc4C(F)(F)F)C3)nc21]

Followed General Procedure E using 3-methyl-2-phenylimidazo[4,5-b]pyrazine-5-carboxylic acid (40 mg, 0.157 mmol, 1.00 equiv) and 3-(piperidin-3-ylmethoxy)-2-(trifluoromethyl)pyridine (45.0 mg, 0.173 mmol, 1.1 equiv) to afford (1-methyl-2-phenyl-1H-imidazo[4,5-b]pyrazin-6-yl)(3-(((2-(trifluoromethyl)pyridin-3-yl)oxy)methyl)piperidin-1-yl)methanone. ¹H NMR (300 MHz, DMSO-d₆) δ 8.57 (d, J=51.5 Hz, 1H), 8.30-8.14 (m, 1H), 8.06-7.94 (m, 2H), 7.77-7.58 (m, 5H), 4.69-4.12 (m, 2H), 4.08-3.87 (m, 4H), 3.88-3.67 (m, 1H), 3.23-2.80 (m, 2H), 2.21-1.36 (m, 5H). MS m/z: 497.0 [M+H]⁺.

### (1-Methyl-2-phenyl-1H-imidazo[4,5-b]pyrazin-5-yl)(3-(((2-(trifluoromethyl)pyridin-3-yl)oxy)methyl)piperidin-1-yl)methanone (57)

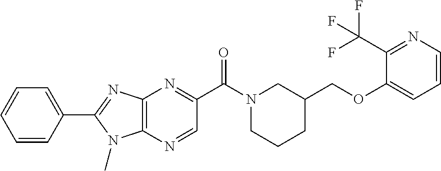 [SMILES: Cn1c(-c2ccccc2)nc2nc(C(=O)N3CCCC(COc4cccnc4C(F)(F)F)C3)cnc21]

Following General Procedure E using 1-methyl-2-phenyl-1H-imidazo[4,5-b]pyrazine-5-carboxylic acid (50 mg, 0.197 mmol, 1.00 equiv) and 3-(piperidin-3-ylmethoxy)-2-(trifluoromethyl)pyridine (56.3 mg, 0.217 mmol, 1.1 equiv) to afford (1-methyl-2-phenyl-1H-imidazo[4,5-b]pyrazin-5-yl)(3-(((2-(trifluoromethyl)pyridin-3-yl)oxy)methyl)piperidin-1-yl)methanone (15 mg, 15.2%) as a white solid. ¹H NMR (300 MHz, DMSO-d₆) δ 8.67 (d, J=31.4 Hz, 1H), 8.32-8.22 (m, 1H), 8.05-7.90 (m, 2H), 7.77-7.55 (m, 5H), 4.60-4.40 (m, 1H), 4.24-4.06 (m, 1H), 3.96 (s, 1H), 3.89-3.71 (m, 4H), 3.20-2.86 (m, 2H), 2.21-2.01 (m, 1H), 2.00-1.38 (m, 4H). MS m/z: 497.0 [M+H]⁺.

### (6-(4-fluorophenyl)-5-methyl-5H-pyrrolo[2,3-b]pyrazin-3-yl)(3-(((2-(trifluoromethyl)pyridin-3-yl)oxy)methyl)piperidin-1-yl)methanone (58)

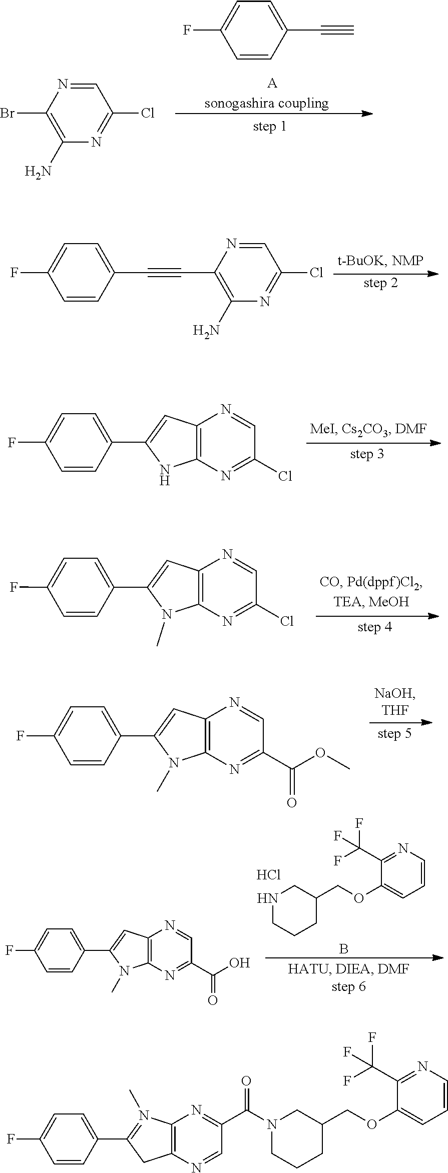

Step 1: 6-chloro-3-((4-fluorophenyl)ethynyl)pyrazin-2-amine: A solution of 3-bromo-6-chloropyrazin-2-amine (500 mg, 2.39 mmol, 1 equiv) and 1-ethynyl-4-fluorobenzene (345 mg, 2.87 mmol, 1.2 equiv) and CuI (15.2 mg, 0.240 mmol, 0.1 equiv) and Pd(PPh₃)₂Cl₂ (168 mg, 0.240 mmol, 0.1 equiv) and TEA (728 mg, 7.19 mmol, 3 equiv) in THF (8 mL) was stirred for 2 h at 80° C. The residue was purified by silica gel column chromatography, eluted with PE/EA (3:1) to afford 6-chloro-3-((4-fluorophenyl)ethynyl)pyrazin-2-amine (450 mg, 75.7%) as a yellow solid. MS m/z: 248 [M+H]⁺.

Step 2: 3-chloro-6-(4-fluorophenyl)-5H-pyrrolo[2,3-b]pyrazine: A solution of 6-chloro-3-((4-fluorophenyl)ethynyl)pyrazin-2-amine (200 mg, 0.808 mmol, 1 equiv) and t-BuOK (181 mg, 1.61 mmol, 2 equiv) in NMP (2 mL) was stirred for 2 h at 80° C. The resulting mixture was purified by reverse flash chromatography with the following conditions: column, C18 silica gel; mobile phase, MeCN in water, 10% to 100% gradient in 15 min; detector, UV 254 nm. This provided 3-chloro-6-(4-fluorophenyl)-5H-pyrrolo[2,3-b]pyrazine (150 mg, 75.0%) as a yellow solid. MS m/z: 248 [M+H]⁺.

Step 3: 3-chloro-6-(4-fluorophenyl)-5-methyl-5H-pyrrolo[2,3-b]pyrazine: A solution of 3-chloro-6-(4-fluorophenyl)-5H-pyrrolo[2,3-b]pyrazine (280 mg, 1.13 mmol, 1 equiv) and MeI (192 mg, 1.35 mmol, 1.2 equiv) and Cs₂CO₃ (1105 mg, 3.39 mmol, 3 equiv) in DMF (3 mL) was stirred for 2 h at room temperature. The reaction mixture was diluted by EtOAc (20 mL), washed by water (2×15 mL) and brine (15 mL), dried over anhydrous sodium sulfate. After filtration, the filtrate was concentrated. The residue was purified by reverse flash chromatography with the following conditions: column, C18 silica gel; mobile phase, MeCN in water, 10% to 50% gradient in 10 min; detector, UV 254 nm. This provided 3-chloro-6-(4-fluorophenyl)-5-methyl-5H-pyrrolo[2,3-b]pyrazine (150 mg, 50.7%) as a white solid. MS m/z: 262 [M+H]⁺.

Step 4: methyl 6-(4-fluorophenyl)-5-methyl-5H-pyrrolo[2,3-b]pyrazine-3-carboxylate: A solution of 3-chloro-6-(4-fluorophenyl)-5-methyl-5H-pyrrolo[2,3-b]pyrazine (80 mg, 0.306 mmol, 1 equiv) and Pd(dppf)Cl₂ (22.3 mg, 0.031 mmol, 0.1 equiv) and TEA (92.8 mg, 0.918 mmol, 3 equiv) in MeOH (3 mL) was stirred for overnight at 100° C. and 50 atm under CO atmosphere. The resulting mixture was filtered, the filter cake was washed with MeOH (2×3 mL). The filtrate was concentrated under reduced pressure. The crude product methyl 6-(4-fluorophenyl)-5-methyl-5H-pyrrolo[2,3-b]pyrazine-3-carboxylate (100 mg, crude) was used in the next step directly without further purification. MS m/z: 286 [M+H]⁺.

Step 5: 6-(4-fluorophenyl)-5-methyl-5H-pyrrolo[2,3-b]pyrazine-3-carboxylic acid: A solution of methyl 6-(4-fluorophenyl)-5-methyl-5H-pyrrolo[2,3-b]pyrazine-3-carboxylate (100 mg, 0.351 mmol, 1 equiv) and NaOH (56.0 mg, 1.40 mmol, 4 equiv) in MeOH (1 mL) and H₂O (1 mL) was stirred for 2 h at 50° C. The mixture was acidified to pH 3 with HCl (1 M). The resulting mixture was extracted with EtOAc (3×10 mL). The combined organic layers were washed with water (2×10 mL) and brine (1×20 mL), dried over anhydrous Na₂SO₄. After filtration, the filtrate was concentrated under reduced pressure. The crude product 6-(4-fluorophenyl)-5-methyl-5H-pyrrolo[2,3-b]pyrazine-3-carboxylic acid (100 mg, crude) was used in the next step directly without further purification. MS m/z: 272 [M+H]⁺.

Step 6: (6-(4-fluorophenyl)-5-methyl-5H-pyrrolo[2,3-b]pyrazin-3-yl)(3-(((2-(trifluoromethyl)pyridin-3-yl)oxy)methyl)piperidin-1-yl)methanone: Followed General Procedure E using 3-(piperidin-3-ylmethoxy)-2-(trifluoromethyl)pyridine hydrochloride (115.14 mg, 0.443 mmol, 1.2 equiv) and 6-(4-fluorophenyl)-5-methyl-5H-pyrrolo[2,3-b]pyrazine-3-carboxylic acid (100 mg, 0.369 mmol, 1.00 equiv) to afford (6-(4-fluorophenyl)-5-methyl-5H-pyrrolo[2,3-b]pyrazin-3-yl)(3-(((2-(trifluoromethyl)pyridin-3-yl)oxy)methyl)piperidin-1-yl)methanone (29.3 mg, 15.4%) as a white solid. ¹H NMR (300 MHz, DMSO-d₆) δ 8.74-8.58 (m, 1H), 8.40-8.20 (m, 1H), 7.94-7.67 (m, 4H), 7.58-7.48 (m, 2H), 7.01-6.95 (m, 1H), 4.82-4.32 (m, 1H), 4.29-4.15 (m, 1H), 4.04-3.89 (m, 3H), 3.76 (s, 2H), 3.27-2.93 (m, 2H), 2.30-2.10 (m, 1H), 2.09-1.85 (m, 2H), 1.78-1.47 (m, 2H). MS m/z: 514.3 [M+H]⁺.

### 2-Chloro-3-({1-[1-(2,2-difluoroethyl)pyrazolo[3,4-b]pyrazin-6-yl]piperidin-3-yl}methoxy) pyridine (59)

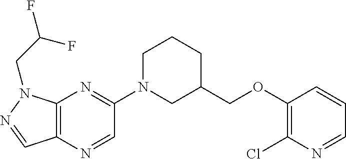 [SMILES: FC(F)Cn1ncc2ncc(N3CCCC(COc4cccnc4Cl)C3)nc21]

Step 1: tert-butyl 3-(((2-chloropyridin-3-yl)oxy)methyl)piperidine-1-carboxylate: Followed General Procedure A using 2-chloropyridin-3-ol (500 mg, 3.86 mmol, 1.00 equiv) and tert-butyl 3-(hydroxymethyl)piperidine-1-carboxylate (997 mg, 4.63 mmol, 1.2 equiv) to afford tert-butyl 3-(((2-chloropyridin-3-yl)oxy)methyl)piperidine-1-carboxylate (800 mg, 63.4%) as a white solid. MS m/z: 327[M+H]⁺.

Step 2: 2-chloro-3-(piperidin-3-ylmethoxy)pyridine hydrochloride: Followed General Procedure B using tert-butyl 3-(((2-chloropyridin-3-yl)oxy)methyl)piperidine-1-carboxylate (400 mg, 1.22 mmol, 1.00 equiv) to afford 2-chloro-3-(piperidin-3-ylmethoxy)pyridine hydrochloride (300 mg). MS m/z: 227 [M+H]⁺.

Step 3: 6-(3-(((2-chloropyridin-3-yl)oxy)methyl)piperidin-1-yl)-1-(2,2-difluoroethyl)-1H-pyrazolo[3,4-b]pyrazine: Followed General Procedure C using 6-chloro-1-(2,2-difluoroethyl)-1H-pyrazolo[3,4-b]pyrazine (60 mg, 0.274 mmol, 1.00 equiv) and 2-chloro-3-(piperidin-3-ylmethoxy)pyridine hydrochloride (86.6 mg, 0.329 mmol, 1.2 equiv) to afford 6-(3-(((2-chloropyridin-3-yl)oxy)methyl)piperidin-1-yl)-1-(2,2-difluoroethyl)-1H-pyrazolo[3,4-b]pyrazine (30 mg, 26.5%) as a white solid. ¹H NMR (300 MHZ, DMSO-d₆) δ 8.45 (s, 1H), 8.12 (s, 1H), 7.99-7.97 (m, 1H), 7.63-7.60 (m, 1H), 7.41-7.37 (m, 1H), 6.65-6.23 (m, 1H), 4.72-4.61 (m, 3H), 4.37-4.33 (m, 1H), 4.17-4.02 (m, 2H), 3.28-3.17 (m, 1H), 3.13-3.06 (m, 1H), 2.18-2.08 (m, 1H), 1.98-1.78 (m, 2H), 1.64-1.48 (m, 2H). MS m/z: 409.2 [M+H]⁺.

### (1R,5S,6S)-3-[6-(1,3,4-thiadiazol-2-yl)pyrazin-2-yl]-6-({[6-(trifluoromethyl)pyridin-2-yl]oxy}methyl)-3-azabicyclo[3.1.0]hexane (60)

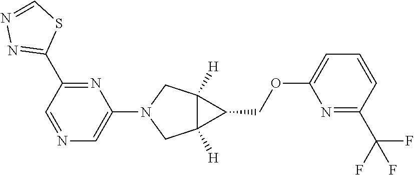 [SMILES: FC(F)(F)c1cccc(OC[C@@H]2[C@H]3CN(c4cncc(-c5nncs5)n4)C[C@@H]23)n1]

Followed General Procedure C using (1R,5S,6S)-6-({[6-(trifluoromethyl)pyridin-2-yl]oxy}methyl)-3-azabicyclo[3.1.0]hexane hydrochloride (22.3 mg, 0.075 mmol, 1 equiv) and 2-chloro-6-(1,3,4-thiadiazol-2-yl)pyrazine (15 mg, 0.075 mmol, 1 equiv) to afford (1R,5S,6S)-3-[6-(1,3,4-thiadiazol-2-yl)pyrazin-2-yl]-6-({[6-(trifluoromethyl)pyridin-2-yl]oxy}methyl)-3-azabicyclo[3.1.0]hexane as a yellow powder (11 mg, 35%). ¹H NMR (500 MHz, CDCl₃) δ 9.18 (s, 1H), 8.83 (s, 1H), 7.95 (s, 1H), 7.72 (t, J=7.8 Hz, 1H), 7.25 (s, 1H, overlapped with CDCl₃ solvent peak), 6.93 (d, J=8.4 Hz, 1H), 4.32 (d, J=7.2 Hz, 2H), 3.90 (d, J=10.4 Hz, 2H), 3.60 (dt, J=10.4, 2.1 Hz, 2H), 1.88 (d, J=3.5 Hz, 2H), 1.21 (m, 1H). MS m/z: 421.4 [M+H]⁺.

### (1R,5S,6S)-3-[1-(2,2-difluoroethyl)-1H-pyrazolo[3,4-b]pyrazin-6-yl]-6-({[5-(trifluoromethyl)pyridin-2-yl]oxy}methyl)-3-azabicyclo[3.1.0]hexane (61)

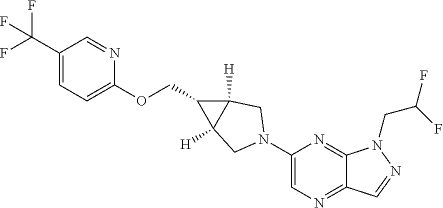 [SMILES: FC(F)Cn1ncc2ncc(N3C[C@@H]4[C@@H](COc5ccc(C(F)(F)F)cn5)[C@@H]4C3)nc21]

Step 1: tert-butyl (1R,5S,6S)-6-({[5-(trifluoromethyl)pyridin-2-yl]oxy}methyl)-3-azabicyclo[3.1.0]hexane-3-carboxylate: Followed General Procedure D using tert-butyl (1R,5S,6S)-6-(hydroxymethyl)-3-azabicyclo[3.1.0]hexane-3-carboxylate (100 mg, 0.47 mmol, 1.00 equiv) and 2-bromo-5-(trifluoromethyl)pyridine (106 mg, 0.47 mmol, 1.0 equiv) to afford tert-butyl (1R,5S,6S)-6-({[5-(trifluoromethyl)pyridin-2-yl]oxy}methyl)-3-azabicyclo[3.1.0]hexane-3-carboxylate (140 mg, 83%) as a colorless oil. MS m/z: 359 [M+H]⁺.

Step 2: (1R,5S,6S)-6-({[5-(trifluoromethyl)pyridin-2-yl]oxy}methyl)-3-azabicyclo[3.1.0]hexane hydrochloride: Followed General Procedure B using tert-butyl (1R,5S,6S)-6-({[5-(trifluoromethyl)pyridin-2-yl]oxy}methyl)-3-azabicyclo[3.1.0]hexane-3-carboxylate (140 mg, 0.39 mmol, 1.00 equiv) to afford 2-[(3-fluoropiperidin-3-yl)methoxy]-6-(trifluoromethyl)pyridine hydrochloride (130 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[5-(trifluoromethyl)pyridin-2-yl]oxy}methyl)-3-azabicyclo[3.1.0]hexane: Followed General Procedure C using (1R,5S,6S)-6-({[5-(trifluoromethyl)pyridin-2-yl]oxy}methyl)-3-azabicyclo[3.1.0]hexane hydrochloride (16.2 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford (1R,5S,6S)-3-[1-(2,2-difluoroethyl)-1H-pyrazolo[3,4-b]pyrazin-6-yl]-6-({[5-(trifluoromethyl)pyridin-2-yl]oxy}methyl)-3-azabicyclo[3.1.0]hexane (17 mg, 70%) as a white solid. ¹H NMR (500 MHZ, CDCl₃) δ 8.46-8.37 (m, 1H), 8.03 (s, 1H), 7.92 (s, 1H), 7.78 (dd, J=8.8, 2.6 Hz, 1H), 6.84 (d, J=8.7 Hz, 1H), 6.22 (tt, J=55.6, 4.5 Hz, 1H), 4.65 (td, J=13.3, 4.5 Hz, 2H), 4.32 (d, J=7.1 Hz, 2H), 3.96 (d, J=10.7 Hz, 2H), 3.66 (dt, J=10.7. 2.1 Hz, 2H), 1.86 (d, J=3.5 Hz, 2H), 1.33-1.26 (m, 1H). MS m/z: 441.4 [M+H]⁺.

### (1R,5S,6S)-3-[1-(2,2-difluoroethyl)-1H-pyrazolo[3,4-b]pyrazin-6-yl]-6-({[5-(trifluoromethyl)pyridin-3-yl]oxy}methyl)-3-azabicyclo[3.1.0]hexane (62)

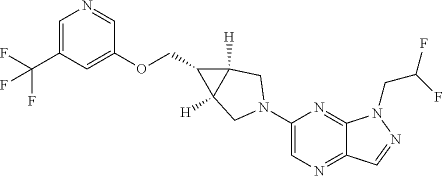 [SMILES: FC(F)Cn1ncc2ncc(N3C[C@@H]4[C@@H](COc5cncc(C(F)(F)F)c5)[C@@H]4C3)nc21]

Step 1: tert-butyl (1R,5S,6S)-6-({[5-(trifluoromethyl)pyridin-3-yl]oxy}methyl)-3-azabicyclo[3.1.0]hexane-3-carboxylate: Followed General Procedure A using tert-butyl (1R,5S,6S)-6-(hydroxymethyl)-3-azabicyclo[3.1.0]hexane-3-carboxylate (100 mg, 0.47 mmol, 1.00 equiv) and 5-(trifluoromethyl)pyridin-3-ol (76.5 mg, 0.47 mmol, 1.0 equiv) to tert-butyl (1R,5S,6S)-6-({[5-(trifluoromethyl)pyridin-3-yl]oxy}methyl)-3-azabicyclo[3.1.0]hexane-3-carboxylate (24 mg, 14%) as a colorless oil. MS m/z: 359 [M+H]⁺.

Step 2: (1R,5S,6S)-6-({[5-(trifluoromethyl)pyridin-3-yl]oxy}methyl)-3-azabicyclo[3.1.0]hexane hydrochloride: Followed General Procedure B using tert-butyl (1R,5S,6S)-6-({[5-(trifluoromethyl)pyridin-3-yl]oxy}methyl)-3-azabicyclo[3.1.0]hexane-3-carboxylate (24 mg, 0.067 mmol, 1.00 equiv) to afford (1R,5S,6S)-6-({[5-(trifluoromethyl)pyridin-3-yl]oxy}methyl)-3-azabicyclo[3.1.0]hexane hydrochloride (20 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[5-(trifluoromethyl)pyridin-3-yl]oxy}methyl)-3-azabicyclo[3.1.0]hexane: Followed General Procedure C using (1R,5S,6S)-6-({[5-(trifluoromethyl)pyridin-3-yl]oxy}methyl)-3-azabicyclo[3.1.0]hexane hydrochloride (16.2 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford (1R,5S,6S)-3-[1-(2,2-difluoroethyl)-1H-pyrazolo[3,4-b]pyrazin-6-yl]-6-({[5-(trifluoromethyl)pyridin-3-yl]oxy}methyl)-3-azabicyclo[3.1.0]hexane (13 mg, 54%) as a white solid. ¹H NMR (500 MHZ, CDCl₃) δ 8.53-8.47 (m, 2H), 8.04 (s, 1H), 7.94 (s, 1H), 7.37 (d, J=2.4 Hz, 1H), 6.23 (tt, J=55.7, 4.5 Hz, 1H), 4.66 (td, J=13.4, 4.6 Hz, 2H), 4.02 (dd, J=13.9, 8.8 Hz, 4H), 3.69 (dt, J=10.8, 2.1 Hz, 2H), 1.95-1.86 (m, 2H), 1.27 (dd, J=7.2, 3.8 Hz, 1H). MS m/z: 441.4 [M+H]⁺.

### (1R,5S,6S)-3-[1-(2,2-difluoroethyl)-1H-pyrazolo[3,4-b]pyrazin-6-yl]-6-({[5-(trifluoromethyl)pyridin-3-yl]oxy}methyl)-3-azabicyclo[3.1.0]hexane (63)

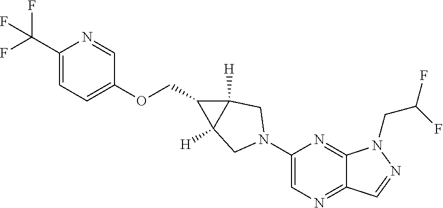 [SMILES: FC(F)Cn1ncc2ncc(N3C[C@@H]4[C@@H](COc5ccc(C(F)(F)F)nc5)[C@@H]4C3)nc21]

Step 1: tert-butyl (1R,5S,6S)-6-({[6-(trifluoromethyl)pyridin-3-yl]oxy}methyl)-3-azabicyclo[3.1.0]hexane-3-carboxylate: Followed General Procedure A using tert-butyl (1R,5S,6S)-6-(hydroxymethyl)-3-azabicyclo[3.1.0]hexane-3-carboxylate (100 mg, 0.47 mmol, 1.00 equiv) and 6-(trifluoromethyl)pyridin-3-ol (76.5 mg, 0.47 mmol, 1.0 equiv) to tert-butyl (1R,5S,6S)-6-({[6-(trifluoromethyl)pyridin-3-yl]oxy}methyl)-3-azabicyclo[3.1.0]hexane-3-carboxylate (106 mg, 63%) as a colorless oil. MS m/z: 359 [M+H]⁺.

Step 2: (1R,5S,6S)-6-({[6-(trifluoromethyl)pyridin-3-yl]oxy}methyl)-3-azabicyclo[3.1.0]hexane hydrochloride: Followed General Procedure B using tert-butyl (1R,5S,6S)-6-({[6-(trifluoromethyl)pyridin-3-yl]oxy}methyl)-3-azabicyclo[3.1.0]hexane-3-carboxylate (106 mg, 0.067 mmol, 1.00 equiv) to afford (1R,5S,6S)-6-({[6-(trifluoromethyl)pyridin-3-yl]oxy}methyl)-3-azabicyclo[3.1.0]hexane hydrochloride (80 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[6-(trifluoromethyl)pyridin-3-yl]oxy}methyl)-3-azabicyclo[3.1.0]hexane: Followed General Procedure C using (1R,5S,6S)-6-({[6-(trifluoromethyl)pyridin-3-yl]oxy}methyl)-3-azabicyclo[3.1.0]hexane hydrochloride (16.2 mg, 0.055 mmol, 1.00 equiv) and 6-chloro-1-(2,2-difluoroethyl)-1H-pyrazolo[3,4-b]pyrazine (12 mg, 0.055 mmol, 1.1 equiv) to afford (1R,5S,6S)-3-[1-(2,2-difluoroethyl)-1H-pyrazolo[3,4-b]pyrazin-6-yl]-6-({[6-(trifluoromethyl)pyridin-3-yl]oxy}methyl)-3-azabicyclo[3.1.0]hexane (10 mg, 41%) as a white solid. ¹H NMR (500 MHZ, CDCl₃) δ 8.39 (d, J=2.8 Hz, 1H), 8.04 (d, J=0.8 Hz, 1H), 7.94 (s, 1H), 7.63 (d, J=8.7 Hz, 1H), 7.31-7.27 (m, 1H), 6.23 (tt, J=55.7, 4.5 Hz, 1H), 4.66 (td, J=13.3, 4.5 Hz, 2H), 4.04 (d, J=7.0 Hz, 2H), 4.00 (d, J=10.7 Hz, 2H), 3.69 (dt, J=10.8, 2.0 Hz, 2H), 1.90 (s, 2H), 1.28 (dq, J=6.8, 3.4 Hz, 1H). MS m/z: 441.4 [M+H]⁺.

### 2-[4,4-Difluoro-3-({[6-(trifluoromethyl)pyridin-2-yl]oxy}methyl)piperidin-1-yl]-6-(1,3,4-thiadiazol-2-yl)pyrazine (64)

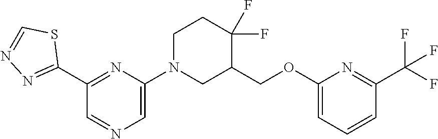 [SMILES: FC(F)(F)c1cccc(OCC2CN(c3cncc(-c4nncs4)n3)CCC2(F)F)n1]

Followed General Procedure C using 2-[(4,4-difluoropiperidin-3-yl)methoxy]-6-(trifluoromethyl)pyridine hydrochloride (20 mg, 0.06 mmol, 1 equiv) and 2-chloro-6-(1,3,4-thiadiazol-2-yl)pyrazine (12 mg, 0.06 mmol, 1 equiv) to afford 2-[4,4-difluoro-3-({[6-(trifluoromethyl)pyridin-2-yl]oxy}methyl)piperidin-1-yl]-6-(1,3,4-thiadiazol-2-yl)pyrazine as a light-yellow oil (9 mg, 33%). ¹H NMR (500 MHZ, CDCl₃) δ 9.19 (s, 1H), 8.89 (s, 1H), 8.32 (s, 1H), 7.75 (t, J=7.8 Hz, 1H), 7.29 (d, J=7.3 Hz, 1H), 6.96 (d, J=8.4 Hz, 1H), 4.78 (dd, J=11.2, 4.1 Hz, 1H), 4.43 (dd, J=11.2, 8.9 Hz, 1H), 4.38 (dt, J=13.8, 3.8 Hz, 1H), 4.33-4.20 (m, 1H), 3.59 (ddd, J=13.9, 10.3, 3.7 Hz, 1H), 3.51 (ddd, J=13.9, 9.6, 1.6 Hz, 1H), 2.61 (ddq, J=18.7, 9.3, 4.7 Hz, 1H), 2.27 (ddt, J=14.0, 9.6, 7.1 Hz, 1H), 2.16-2.01 (m, 1H). MS m/z: 459.4 [M+H]⁺.

### 2-[3-Fluoro-3-({[6-(trifluoromethyl)pyridin-2-yl]oxy}methyl)piperidin-1-yl]-6-(1,3,4-thiadiazol-2-yl)pyrazine (65)

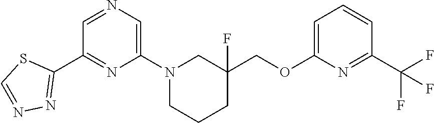 [SMILES: FC1(COc2cccc(C(F)(F)F)n2)CCCN(c2cncc(-c3nncs3)n2)C1]

Followed General Procedure C using 2-[(3-fluoropiperidin-3-yl)methoxy]-6-(trifluoromethyl)pyridine hydrochloride (19 mg, 0.06 mmol, 1 equiv) and 2-chloro-6-(1,3,4-thiadiazol-2-yl)pyrazine (12 mg, 0.06 mmol, 1 equiv) to afford 2-[3-fluoro-3-({[6-(trifluoromethyl)pyridin-2-yl]oxy}methyl)piperidin-1-yl]-6-(1,3,4-thiadiazol-2-yl)pyrazine as a light-yellow oil (9 mg, 33%). ¹H NMR (500 MHZ, CDCl₃) δ 9.19 (s, 1H), 8.83 (s, 1H), 8.30 (s, 1H), 7.76 (t, J=7.8 Hz, 1H), 7.30 (d, J=7.2 Hz, 1H), 7.04 (d, J=8.4 Hz, 1H), 4.57 (d, J=3.3 Hz, 1H), 4.53 (s, 1H), 4.37 (dd, J=14.1, 8.8 Hz, 1H), 4.22-4.11 (m, 1H), 3.58 (dd, J=26.8, 14.1 Hz, 1H), 3.38-3.29 (m, 1H), 2.16 (t, J=11.5 Hz, 1H), 2.03-1.87 (m, 2H), 1.81 (dt, J=13.4, 4.5 Hz, 1H). MS m/z: 441.4 [M+H]⁺.

### 1-(2,2-Difluoroethyl)-6-(3-(2-(2-(trifluoromethyl)phenyl)propyl)piperidin-1-yl)-1H-pyrazolo[3,4-b]pyrazine (66)

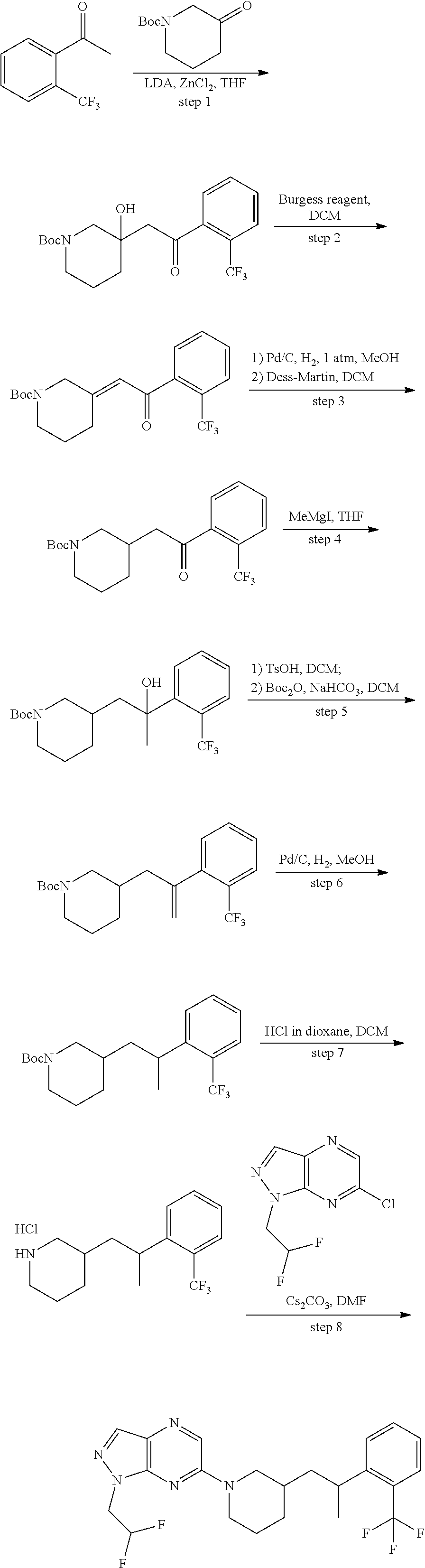

Step 1: tert-butyl 3-hydroxy-3-(2-oxo-2-(2-(trifluoromethyl)phenyl)ethyl)piperidine-1-carboxylate: To a stirred solution of 1-(2-(trifluoromethyl)phenyl)ethan-1-one (4.00 g, 21.3 mmol, 1.00 equiv) in THF (20.0 mL) was added LDA (4.56 g, 42.5 mmol, 2.00 equiv) at −78° C. The resulting mixture was allowed to stir at this temperature for 0.5 h, followed by adding tert-butyl 3-oxopiperidine-1-carboxylate (4.24 g, 21.3 mmol, 1.00 equiv), and ZnCl (0.7 M in THF, 30.4 mL, 1.00 equiv.) dropwise at −78° C. under argon atmosphere. After reaction for another 0.5 h, the reaction was quenched with sat. NH₄Cl (aq.) at room temperature. The resulting mixture was extracted with EtOAc (3×50 mL). The combined organic layers were washed with water (2×40 mL) and brine (40 mL), dried over anhydrous Na₂SO₄. After filtration, the filtrate was concentrated under reduced pressure. The residue was purified by silica gel column chromatography, eluted with EtOAc/PE (1/2) to afford tert-butyl 3-hydroxy-3-(2-oxo-2-(2-(trifluoromethyl)phenyl)ethyl)piperidine-1-carboxylate (2.00 g, 24.3%) as a white solid. MS m/z: 389 [M+H]⁺.

Step 2: tert-butyl (E)-3-(2-oxo-2-(2-(trifluoromethyl)phenyl)ethylidene)piperidine-1-carboxylate: A mixture of tert-butyl 3-hydroxy-3-(2-oxo-2-(2-(trifluoromethyl)phenyl)ethyl)piperidine-1-carboxylate (2.00 g, 5.16 mmol, 1.00 equiv) and Burgess reagent (6.15 g, 25.8 mmol, 5.00 equiv) in DCM (4.00 mL) was stirred for overnight at room temperature under air atmosphere. The resulting mixture was concentrated under reduced pressure. The resulting mixture was extracted with EtOAc (3×50 mL). The combined organic layers were washed with water (3×40 mL) and brine, dried over anhydrous Na₂SO₄. After filtration, the filtrate was concentrated under reduced pressure. The residue was purified by silica gel column chromatography, eluted with EtOAc/PE (1/1) to afford tert-butyl (E)-3-(2-oxo-2-(2-(trifluoromethyl)phenyl)ethylidene)piperidine-1-carboxylate. MS m/z: 370 [M+H]⁺.

Step 3: tert-butyl 3-(2-oxo-2-(2-(trifluoromethyl)phenyl)ethyl)piperidine-1-carboxylate: To the solution of tert-butyl (E)-3-(2-oxo-2-(2-(trifluoromethyl)phenyl)ethylidene)piperidine-1-carboxylate (1.2 g, 3.25 mmol, 1 equiv) in McOH (20 mL) was added Pd/C (120 mg, 10% Pd on carbon, wetted with water). The resulted mixture was hydrogenated overnight at room temperature. Desired product could be detected by LCMS. The reaction system was filtrated through celite and the filtrate was concentrated. The crude product was dissolved in DCM (20 mL), and was added with Dess-Martin reagent (2.76 g, 6.50 mmol, 2 eq.). The resulting mixture was allowed to react at room temperature for 2 h, filtrated, and concentrated. The residue was purified by silica gel column chromatography, eluted with EtOAc/PE (1/1) to afford tert-butyl 3-(2-oxo-2-(2-(trifluoromethyl)phenyl)ethyl)piperidine-1-carboxylate (550 mg) was used directly for next step. MS m/z: 372 [M+H]⁺.

Step 4: tert-butyl 3-(2-hydroxy-2-(2-(trifluoromethyl)phenyl)propyl)piperidine-1-carboxylate: To a stirred solution of tert-butyl 3-(2-oxo-2-(2-(trifluoromethyl)phenyl)ethyl)piperidine-1-carboxylate (550 mg, 1.48 mmol, 1.00 equiv) in THF (5.00 mL) was added CH₃MgI (985 mg, 5.92 mmol, 4.00 equiv) dropwise at 0° C. under air atmosphere. The reaction was quenched with water at 0° C. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with EtOAc/PE (1/1) to afford tert-butyl 3-(2-hydroxy-2-(2-(trifluoromethyl)phenyl)propyl)piperidine-1-carboxylate (280 mg, 48.8%) as a white solid. MS m/z: 388 [M+H]⁺.

Step 5: tert-butyl 3-(2-(2-(trifluoromethyl)phenyl)allyl)piperidine-1-carboxylate: A mixture of tert-butyl 3-(2-hydroxy-2-(2-(trifluoromethyl)phenyl)propyl)piperidine-1-carboxylate (280 mg, 0.723 mmol, 1.00 equiv) and TsOH (622 mg, 3.62 mmol, 5.00 equiv) in Toluene (3.00 mL) was stirred for 2 h at 100° C. under air atmosphere. To the above mixture was added NaHCO₃(364 mg, 4.34 mmol, 6.00 equiv) and Boc₂O (237 mg, 1.09 mmol, 1.50 equiv) at room temperature. The resulting mixture was stirred for additional 2 h at room temperature. The resulting mixture was extracted with EtOAc (3×20 mL). The combined organic layers were washed with water (2×20 mL), dried over anhydrous Na₂SO₄. After filtration, the filtrate was concentrated under reduced pressure. The residue was purified by silica gel column chromatography, eluted with EtOAc/PE (1/1) to afford tert-butyl 3-(2-(2-(trifluoromethyl)phenyl)allyl)piperidine-1-carboxylate (220 mg, 82.4%) as a white solid. MS m/z: 370 [M+H]⁺.

Step 6: tert-butyl 3-(2-(2-(trifluoromethyl)phenyl)propyl)piperidine-1-carboxylate: To the solution of tert-butyl 3-(2-(2-(trifluoromethyl)phenyl)allyl)piperidine-1-carboxylate (220 mg, 0.596 mmol, 1.00 equiv) in MeOH (4.00 mL) was added Pd/C (6.34 mg, 10% Pd on carbon, wetted with water). The resulted mixture was hydrogenated overnight at room temperature. Desired product could be detected by LCMS. The reaction system was filtrated through celite and the filtrate was concentrated. The crude product tert-butyl 3-(2-(2-(trifluoromethyl)phenyl)propyl)piperidine-1-carboxylate (200 mg) was used directly for next step. MS m/z: 372 [M+H]⁺.

Step 7: 3-(2-(2-(trifluoromethyl)phenyl)propyl)piperidine hydrochloride: Followed General Procedure B using tert-butyl 3-(2-(2-(trifluoromethyl)phenyl)propyl)piperidine-1-carboxylate (200 mg) to afford the crude product 3-(2-(2-(trifluoromethyl)phenyl)propyl)piperidine hydrochloride (200 mg) was used for next step without further purification. MS m/z: 261 [M+H]⁺.

Step 8: 1-(2,2-difluoroethyl)-6-(3-(2-(2-(trifluoromethyl)phenyl)propyl)piperidin-1-yl)-1H-pyrazolo[3,4-b]pyrazine: Followed General Procedure C using 3-(2-(2-(trifluoromethyl)phenyl)propyl)piperidine hydrochloride (100 mg, 0.325 mmol, 1.00 equiv) and 6-chloro-1-(2,2-difluoroethyl)-1H-pyrazolo[3,4-b]pyrazine (71.0 mg, 0.325 mmol, 1.00 equiv). The residue was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(3-(2-(2-(trifluoromethyl)phenyl)propyl)piperidin-1-yl)-1H-pyrazolo[3,4-b]pyrazine (25.0 mg, 17.0%) as a white solid. ¹H NMR (400 MHZ, DMSO-d₆) δ 8.35 (d, J=3.9 Hz, 1H), 8.10 (d, J=1.0 Hz, 1H), 7.69-7.62 (m, 3H), 7.39 (t, J=7.6 Hz, 1H), 6.56-6.28 (m, 1H), 4.71-4.60 (m, 2H), 4.47-4.25 (m, 2H), 3.26 (s, 1H), 3.13-3.01 (m, 1H), 2.90-2.62 (m, 1H), 1.81 (s, 1H). 1.72-1.54 (m, 3H), 1.44 (d, J=14.1 Hz, 2H), 1.29-1.15 (m, 4H). MS m/z: 454 [M+H]⁺.

### (1-phenyl-1H-pyrazolo[3,4-b]pyrazin-5-yl)(3-((o-tolyloxy)methyl)piperidin-1-yl)methanone (67)

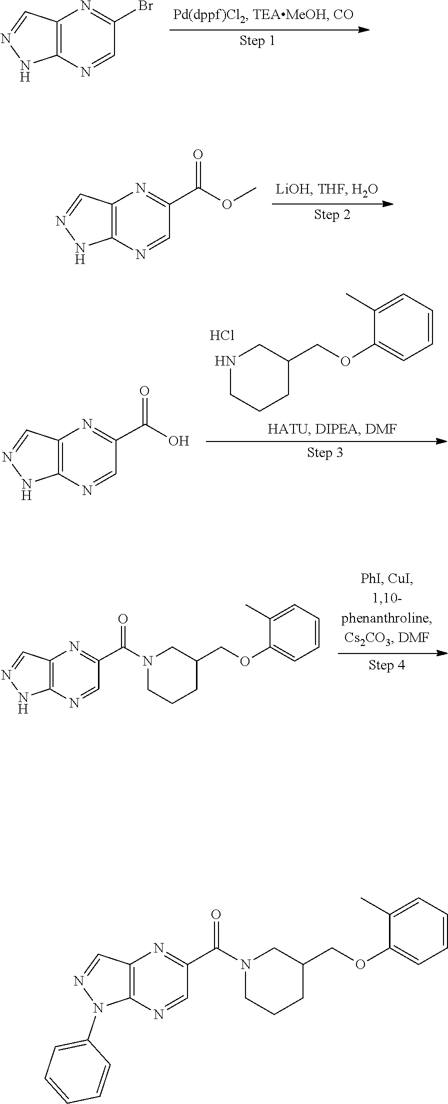

Step 1: methyl 1H-pyrazolo[3,4-b]pyrazine-5-carboxylate: A solution of 5-bromo-1H-pyrazolo[3,4-b]pyrazine (300 mg, 1.51 mmol, 1.00 equiv), Et₃N (459 mg, 4.54 mmol, 3 equiv) and Pd(dppf)Cl₂ (110 mg, 0.151 mmol, 0.1 equiv) in MeOH (10 mL) was stirred for overnight at 100° C. under carbon monoxide atmosphere. The resulting mixture was concentrated under vacuum. The residue was purified by silica gel column chromatography, eluted with PE/EA (1:2) to afford methyl 1H-pyrazolo[3,4-b]pyrazine-5-carboxylate (250 mg, 93.0%) as a yellow solid. MS m/z: 179 [M+H]⁺.

Step 2: 1H-pyrazolo[3,4-b]pyrazine-5-carboxylic acid: To a stirred solution of methyl 1H-pyrazolo[3,4-b]pyrazine-5-carboxylate (250 mg, 1.40 mmol, 1 equiv) in THF (3 mL) and H₂O (3 mL) was added LiOH·H₂O (58.8 mg, 0.4 mmol, 2 equiv) at 0° C. The resulting mixture was stirred for 2 h at 0° C. under. The resulting mixture was acidified by HCl (3 M) to PH ˜3. The aqueous phase was extracted by EtOAc (3×10 mL). The combined organic layers were washed by water (2×15 mL) and brine (1×15 mL), dried over anhydrous Na₂SO₄. After filtration, the resulting mixture was concentrated under reduced pressure to afford 1H-pyrazolo[3,4-b]pyrazine-5-carboxylic acid (250 mg, crude) as a white solid. MS m/z: 166 [M+H]⁺.

Step 3: (1H-pyrazolo[3,4-b]pyrazin-5-yl)(3-((o-tolyloxy)methyl)piperidin-1-yl)methanone: To a stirred mixture of 1H-pyrazolo[3,4-b]pyrazine-5-carboxylic acid (100 mg, 0.606 mmol, 1.00 equiv) and 3-((o-tolyloxy)methyl)piperidine hydrochloride (146 mg, 0.606 mmol, 1.00 equiv) in DMF (3.00 mL) were added HATU (380 mg, 0.606 mmol, 1.00 equiv) and DIPEA (234 mg, 1.82 mmol, 3.00 equiv), The resulting mixture was stirred for 1 h at room temperature under argon atmosphere. The resulting mixture was diluted with water (10 mL). The resulting mixture was extracted with EtOAc (3×20 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, ACN in water, 10% to 95% gradient in 10 min; detector, UV 254 nm. This provided (1H-pyrazolo[3,4-b]pyrazin-5-yl)(3-((o-tolyloxy)methyl)piperidin-1-yl)methanone (100 mg, 47.0%) as a white solid. MS m/z: 352 [M+H]⁺.

Step 4: (1-phenyl-1H-pyrazolo[3,4-b]pyrazin-5-yl)(3-((o-tolyloxy)methyl)piperidin-1-yl)methanone: To the solution of (1H-pyrazolo[3,4-b]pyrazin-5-yl)(3-((o-tolyloxy)methyl)piperidin-1-yl)methanone (100 mg, 0.284 mmol, 1 equiv) and iodobenzene (116 mg, 0.570 mmol, 2.0 equiv) in DMF (2 mL) were added CuI (5.4 mg, 0.028 mmol, 0.1 equiv), 1,10-phenanthroline (5.2 mg, 0.028, 0.1 equiv.) and Cs₂CO₃ (323 mg. 0.852 mmol, 3 equiv) under N₂ atmosphere. The result mixture was heated to 100° C. and stirred overnight. Desired product could be detected by LCMS. The reaction mixture was diluted by EtOAc (20 mL), washed by water (2×15 mL) and brine (1×15 mL), dried over anhydrous sodium sulfate. After filtration, the filtrate was concentrated. The residue was purified by silica gel column chromatography, eluted with EtOAc/PE=1/2, to afford impure product. This was further purified by reverse flash chromatography with the following conditions (column, C18 gel; mobile phase, B phase: MeCN, A phase: water; 35% to 75% B gradient in 20 min; detector: UV 254/220 nm). This provided (1-phenyl-1H-pyrazolo[3,4-b]pyrazin-5-yl)(3-((o-tolyloxy)methyl)piperidin-1-yl)methanone (11.2 mg, 9.20%) as a white solid. ¹H NMR (400 MHZ, CD₃OD) δ 8.91-8.70 (m, 1H), 8.59-8.33 (m, 1H), 8.29-8.19 (m, 2H), 7.61-7.52 (m, 2H), 7.43-7.34 (m, 1H), 7.18-6.64 (m, 4H), 4.77-4.20 (m, 1H), 4.08-3.71 (m, 3H), 3.47-3.34 (m, 1H), 3.28-3.03 (m, 1H), 2.32-2.12 (m, 2H), 2.08-1.67 (m, 3H), 1.66-1.49 (m, 3H). MS m/z: 428.1 [M+H]⁺.

### (5-methyl-6-phenyl-5H-pyrrolo[2,3-b]pyrazin-2-yl)(3-(((2-(trifluoromethyl)pyridin-3-yl)oxy)methyl)piperidin-1-yl)methanone (68)

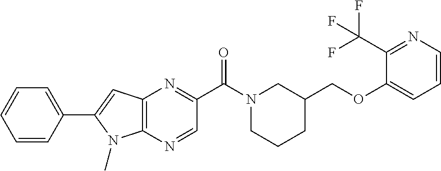 [SMILES: Cn1c(-c2ccccc2)cc2nc(C(=O)N3CCCC(COc4cccnc4C(F)(F)F)C3)cnc21]

Followed General Procedure E using 5-methyl-6-phenyl-5H-pyrrolo[2,3-b]pyrazine-2-carboxylic acid (150 mg, 0.592 mmol, 1.00 equiv) and 3-(piperidin-3-ylmethoxy)-2-(trifluoromethyl)pyridine (154. mg, 0.592 mmol, 1 equiv). The residue was purified by reverse flash chromatography with the following conditions: column, C18 silica gel; mobile phase, ACN in water, 10% to 50% gradient in 10 min; detector, UV 254 nm. This provided (5-methyl-6-phenyl-5H-pyrrolo[2,3-b]pyrazin-2-yl)(3-(((2-(trifluoromethyl)pyridin-3-yl)oxy)methyl)piperidin-1-yl)methanone (20.1 mg, 6.85%) as a yellow solid. ¹H NMR (300 MHz, DMSO-d₆) δ 8.53-8.33 (m, 1H), 8.31-8.16 (m, 1H), 7.88-7.69 (m, 3H), 7.68-7.50 (m, 4H), 6.93-6.57 (m, 1H), 4.67-3.94 (m, 3H), 3.97-3.75 (m, 4H), 3.24-2.82 (m, 2H), 2.20-1.99 (m, 1H), 1.97-1.37 (m, 4H). MS m/z: 496.0 [M+H]⁺.

### 6-(3-(((3-Chloropyrazin-2-yl)oxy)methyl)piperidin-1-yl)-1-(2,2-difluoroethyl)-1H-pyrazolo[3,4-b]pyrazine (69)

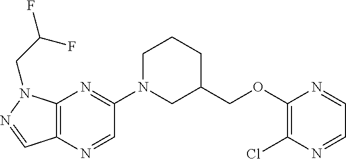 [SMILES: FC(F)Cn1ncc2ncc(N3CCCC(COc4nccnc4Cl)C3)nc21]

Step 1: tert-butyl 3-(((3-chloropyrazin-2-yl)oxy)methyl)piperidine-1-carboxylate: Followed General Procedure A using 3-chloropyrazin-2-ol (500 mg, 3.83 mmol, 1 equiv) and tert-butyl 3-(hydroxymethyl)piperidine-1-carboxylate (824 mg, 3.83 mmol, 1 equiv to afford tert-butyl 3-(((3-chloropyrazin-2-yl)oxy)methyl)piperidine-1-carboxylate (350 mg, 27.87%) as a white solid. MS m/z: 328[M+H]⁺.

Step 2: 2-chloro-3-(piperidin-3-ylmethoxy)pyrazine hydrochloride: Followed General Procedure B using tert-butyl 3-(((3-chloropyrazin-2-yl)oxy)methyl)piperidine-1-carboxylate (350 mg, 1.07 mmol, 1.00 equiv) to afford the crude product 2-chloro-3-(piperidin-3-ylmethoxy)pyrazine hydrochloride (220 mg). MS m/z: 228 [M+H]⁺.

Step 3: 6-(3-(((3-chloropyrazin-2-yl)oxy)methyl)piperidin-1-yl)-1-(2,2-difluoroethyl)-1H-pyrazolo[3,4-b]pyrazine: Followed General Procedure C using 6-chloro-1-(2,2-difluoroethyl)-1H-pyrazolo[3,4-b]pyrazine (60 mg, 0.274 mmol, 1.00 equiv) and 2-chloro-3-(piperidin-3-ylmethoxy)pyrazine hydrochloride (86.6 mg, 0.329 mmol, 1.2 equiv). The residue was purified by reverse flash chromatography with the following conditions: column, C18 silica gel; mobile phase, MeCN in water, 0% to 100% gradient in 30 min; detector, UV 254 nm. This provided 6-(3-(((2-chloropyridin-3-yl)oxy)methyl)piperidin-1-yl)-1-(2,2-difluoroethyl)-1H-pyrazolo[3,4-b]pyrazine (30 mg, 26.5%) as a white solid. ¹H NMR (300 MHz, DMSO-d₆) δ 8.45 (s, 1H), 8.21 (d, J=2.7 Hz, 1H), 8.12 (s, 1H), 8.07 (d, J=2.7 Hz, 1H), 6.42 (tt, J=55.0, 3.9 Hz, 1H), 4.73-4.53 (m, 3H), 4.45-4.26 (m, 3H), 3.30-3.17 (m, 1H), 3.17-3.06 (m, 1H), 2.22-2.09 (m, 1H), 1.97-1.74 (m, 2H), 1.65-1.40 (m, 2H). MS m/z: 410.0 [M+H]⁺.

### 1-(2,2-Difluoroethyl)-6-(3-(((3-methylpyrazin-2-yl)oxy)methyl)piperidin-1-yl)-1H-pyrazolo[3,4-b]pyrazine (70)

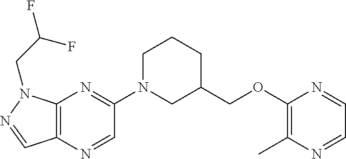 [SMILES: Cc1nccnc1OCC1CCCN(c2cnc3cnn(CC(F)F)c3n2)C1]

Step 1: tert-butyl 3-(((3-methylpyrazin-2-yl)oxy)methyl)piperidine-1-carboxylate: Followed General Procedure A using tert-butyl 3-(hydroxymethyl)piperidine-1-carboxylate (1 g, 4.64 mmol, 1.00 equiv) and 3-methylpyrazin-2-ol (0.61 g, 5.57 mmol, 1.2 equiv) to afford tert-butyl 3-(((3-methylpyrazin-2-yl)oxy)methyl)piperidine-1-carboxylate (700 mg, 49.0%) as a white solid. MS m/z: 308 [M+H]⁺.

Step 2: 2-methyl-3-(piperidin-3-ylmethoxy)pyrazine hydrochloride: Followed General Procedure B using tert-butyl 3-(((3-methylpyrazin-2-yl)oxy)methyl)piperidine-1-carboxylate (700 mg, 2.27 mmol, 1 equiv) to afford the crude product 2-methyl-3-(piperidin-3-ylmethoxy)pyrazine hydrochloride (550 mg) as a white solid. MS m/z: 208 [M+H]⁺.

Step 3: 1-(2,2-difluoroethyl)-6-(3-(((3-methylpyrazin-2-yl)oxy)methyl)piperidin-1-yl)-1H-pyrazolo[3,4-b]pyrazine: Followed General Procedure C using 2-methyl-3-(piperidin-3-ylmethoxy)pyrazine hydrochloride (100 mg, 0.410 mmol, 1.00 equiv) and 6-chloro-1-(2,2-difluoroethyl)-1H-pyrazolo[3,4-b]pyrazine (107 mg, 0.492 mmol, 1.2 equiv). The residue was purified by reverse flash chromatography with the following conditions: column, C18 silica gel; mobile phase, MeCN in Water (0.1% NH₃·H₂O), 10% to 100% gradient in 30 min; detector, UV 254 nm. This provided 1-(2,2-difluoroethyl)-6-(3-(((3-methylpyrazin-2-yl)oxy)methyl)piperidin-1-yl)-1H-pyrazolo[3,4-b]pyrazine (96.3 mg, 59.97%) as a white solid. ¹H NMR (400 MHZ, DMSO-d₆) δ 8.46 (s, 1H), 8.11 (s, 1H), 8.04 (d, J=2.9 Hz, 1H), 8.00 (d, J=2.9 Hz, 1H), 6.60-6.40 (m, 1H), 4.71-4.57 (m, 3H), 4.39-4.28 (m, 2H), 4.27-4.17 (m, 1H), 3.27-3.17 (m, 1H), 3.14-3.03 (m, 1H), 2.47 (s, 3H), 2.09 (d, J=15.2 Hz, 1H), 1.96-1.87 (m, 1H), 1.84-1.76 (m, 1H), 1.63-1.41 (m, 2H). MS m/z: 390.2 [M+H]⁺.

### 1-(4-(6-(3-(2-(Trifluoromethyl)phenethyl)piperidin-1-yl)pyrazin-2-yl)piperidin-1-yl)ethan-1-one (70)

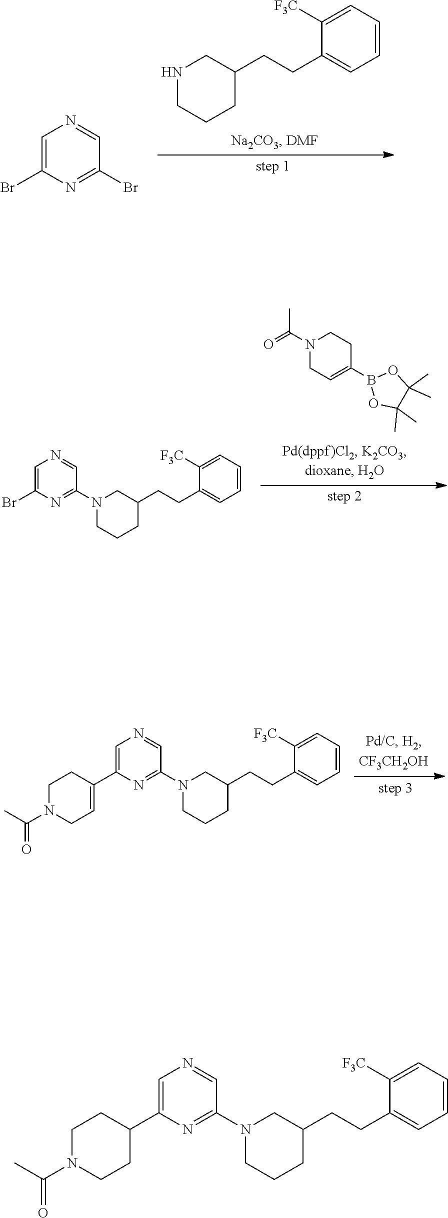 [SMILES: Brc1cncc(Br)n1 ; FC(F)(F)c1ccccc1CCC1CCCNC1 ; CC(=O)N1CC=C(B2OC(C)(C)C(C)(C)O2)CC1 ; FC(F)(F)c1ccccc1CCC1CCCN(c2cncc(Br)n2)C1 ; CC(=O)N1CC=C(c2cncc(N3CCCC(CCc4ccccc4C(F)(F)F)C3)n2)CC1 ; CC(=O)N1CCC(c2cncc(N3CCCC(CCc4ccccc4C(F)(F)F)C3)n2)CC1]

Step 1. 2-bromo-6-(3-(2-(trifluoromethyl)phenethyl)piperidin-1-yl)pyrazine: A solution of 2,6-dibromopyrazine (220 mg, 0.925 mmol, 1 equiv), 3-(2-(trifluoromethyl)phenethyl)piperidine (262 mg, 1.02 mmol, 1.1 equiv) and Na₂CO₃ (196 mg, 1.85 mmol, 2 equiv) in DMF (2 mL) was stirred for 3 h at 100° C. The resulting mixture was diluted with EtOAc (30 mL). The organic layer was washed with water (3×20 mL), dried over anhydrous Na₂SO₄. After filtration, the filtrate was concentrated under reduced pressure. The residue was purified by silica gel column chromatography, eluted with PE/EA (3:1) to afford 2-bromo-6-(3-(2-(trifluoromethyl)phenethyl)piperidin-1-yl)pyrazine (150 mg, 39.2%) as an off-white solid. MS m/z: 414 [M+H]⁺.

Step 2: 1-(4-(6-(3-(2-(trifluoromethyl)phenethyl)piperidin-1-yl)pyrazin-2-yl)-3,6-dihydropyridin-1(2H)-yl)ethan-1-one: A solution of 2-bromo-6-(3-(2-(trifluoromethyl)phenethyl)piperidin-1-yl)pyrazine (150 mg, 0.362 mmol, 1 equiv), 1-(4-(4,4,5,5-tetramethyl-1,3,2-dioxaborolan-2-yl)-3,6-dihydropyridin-1(2H)-yl)ethan-1-one (136 mg, 0.543 mmol, 1.5 equiv), Pd(dppf)Cl₂ (26.5 mg, 0.036 mmol, 0.1 equiv) and K₂CO₃ (100 mg, 0.724 mmol, 2 equiv) in dioxane (4 mL)/H₂O (0.8 mL) was stirred for overnight at 80° C. under argon atmosphere. The resulting mixture was concentrated under vacuum. The residue was purified by silica gel column chromatography, eluted with PE/EA (5:1) to afford 1-(4-(6-(3-(2-(trifluoromethyl)phenethyl)piperidin-1-yl)pyrazin-2-yl)-3,6-dihydropyridin-1(2H)-yl)ethan-1-one (120 mg, 72.3%) as a white solid. MS m/z: 459 [M+H]⁺.

Step 3: 1-(4-(6-(3-(2-(trifluoromethyl)phenethyl)piperidin-1-yl)pyrazin-2-yl)piperidin-1-yl)ethan-1-one: A solution of 1-(4-(6-(3-(2-(trifluoromethyl)phenethyl)piperidin-1-yl)pyrazin-2-yl)-3,6-dihydropyridin-1(2H)-yl)ethan-1-one (60 mg, 0.131 mmol, 1.00 equiv) and Pd/C (1.39 mg, 0.013 mmol, 0.1 equiv) in CF₃CH₂OH (5 mL) was stirred for overnight at room temperature under hydrogen atmosphere. The precipitated solids were collected by filtration and washed with MeOH (4×20 mL). The residue was purified by reverse flash chromatography with the following conditions: column, C18 silica gel; mobile phase, MeCN in water, 10% to 100% gradient in 10 min; detector, UV 254 nm. This provided 1-(4-(6-(3-(2-(trifluoromethyl)phenethyl)piperidin-1-yl)pyrazin-2-yl)piperidin-1-yl)ethan-1-one (20 mg, 32.9%) as a white solid. ¹H NMR (400 MHZ, DMSO-d₆) δ 8.10 (s, 1H), 7.71 (s, 1H), 7.61 (t, J=7.3 Hz, 2H), 7.26 (d, J=8.6 Hz, 1H), 7.09 (t, J=7.5 Hz, 1H), 4.48 (d, J=12.8 Hz, 2H), 4.19 (d, J=13.0 Hz, 1H), 4.03-4.07 (m, 1H), 3.99 (t, J=8.8 Hz, 1H), 3.93-3.83 (d, J=13.5 Hz, 1H), 3.15-3.04 (m, 1H), 2.99-2.90 (m, 1H), 2.89-2.80 (m, 1H), 2.78-2.70 (m, 1H), 2.64-2.55 (m, 1H), 2.01 (s, 4H), 1.92-1.84 (m, 1H), 1.84-1.72 (m, 3H), 1.69-1.60 (m, 1H), 1.57-1.36 (m, 3H). MS m/z: 463.1 [M+H]⁺.

### 1-(4-(5-methyl-3-(3-(((6-(trifluoromethyl)pyridin-2-yl)oxy)methyl)piperidin-1-yl)-5H-pyrrolo[2,3-b]pyrazin-6-yl)piperidin-1-yl)ethan-1-one (72)

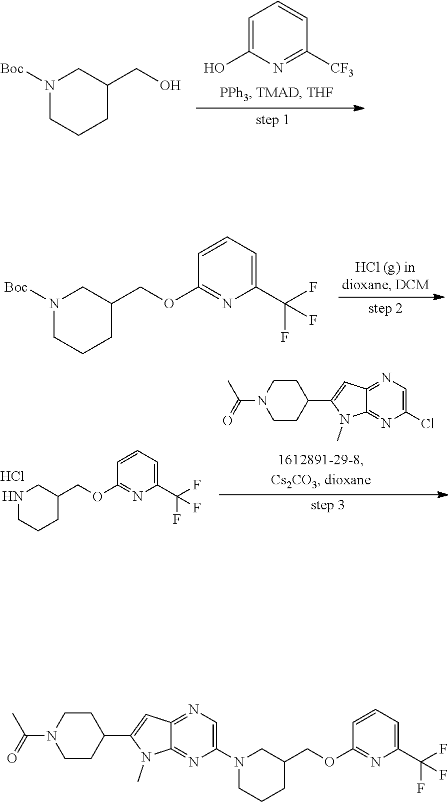

Step 1: tert-butyl 3-(((6-(trifluoromethyl)pyridin-2-yl)oxy)methyl)piperidine-1-carboxylate: To a stirred mixture of tert-butyl 3-(hydroxymethyl)piperidine-1-carboxylate (1 g, 4.64 mmol, 1.00 equiv), 6-(trifluoromethyl)pyridin-2-ol (0.91 g, 5.57 mmol, 1.2 equiv) and PPh₃ (1.95 g, 7.43 mmol, 1.6 equiv) in THF (10 mL) was added TMAD (1.28 g, 7.43 mmol, 1.6 equiv) in portions at 0° C. The resulting mixture was warmed to room temperature and stirred for overnight at room temperature. Desired product could be detected by LCMS. The resulting mixture was concentrated under reduced pressure. The residue was purified by silica gel column chromatography, eluted with EtOAc/PE (1/4) to afford tert-butyl 3-(((6-(trifluoromethyl)pyridin-2-yl)oxy)methyl)piperidine-1-carboxylate (700 mg, 41.8%) as a white solid. MS m/z: 361 [M+H]⁺.

Step 2: 2-(piperidin-3-ylmethoxy)-6-(trifluoromethyl)pyridine hydrochloride: A solution of tert-butyl 3-(((6-(trifluoromethyl)pyridin-2-yl)oxy)methyl)piperidine-1-carboxylate (700 mg, 1.94 mmol, 1 equiv) and 4N HCl (gas) 1,4-dioxane solution (5 mL) in DCM (5 mL) was stirred for 2 h at room temperature. The desired product could be detected by LCMS. The resulting mixture was concentrated under vacuum to afford 2-(piperidin-3-ylmethoxy)-6-(trifluoromethyl)pyridine hydrochloride (550 mg, crude) as a white solid. MS m/z: 261 [M+H]⁺.

Step 3: 1-(4-(5-methyl-3-(3-(((6-(trifluoromethyl)pyridin-2-yl)oxy)methyl)piperidin-1-yl)-5H-pyrrolo[2,3-b]pyrazin-6-yl)piperidin-1-yl)ethan-1-one: To a stirred solution of 2-(piperidin-3-ylmethoxy)-6-(trifluoromethyl)pyridine hydrochloride (60.8 mg, 0.205 mmol, 1.2 equiv) and 1-(4-(3-chloro-5-methyl-5H-pyrrolo[2,3-b]pyrazin-6-yl)piperidin-1-yl)ethan-1-one (50 mg, 0.171 mmol, 1.00 equiv) in dioxane (1 mL) were added Cs₂CO₃ (83.4 mg, 0.257 mmol, 1.5 equiv) and Pd-PEPPSI-IPentCl 2-methylpyridine (o-picoline) (14.3 mg, 0.017 mmol, 0.1 equiv). The resulting mixture was stirred for 16 h at 90° C. The desired product could be detected by LCMS. The residue was purified by reverse flash chromatography with the following conditions: column, C18 silica gel; mobile phase, MeCN in Water (0.1% NH₃·H₂O), 10% to 100% gradient in 30 min; detector, UV 254 nm. This provided 1-(4-(5-methyl-3-(3-(((6-(trifluoromethyl)pyridin-2-yl)oxy)methyl)piperidin-1-yl)-5H-pyrrolo[2,3-b]pyrazin-6-yl)piperidin-1-yl)ethan-1-one (19.8 mg, 21.68%) as a yellow solid. ¹H NMR (400 MHZ, DMSO-d₆) δ 8.14-8.07 (m, 1H), 8.04-7.93 (m, 1H), 7.53-7.44 (m, 1H), 7.29-7.18 (m, 1H), 6.19 (d, J=3.3 Hz, 1H), 4.52 (d, J=13.1 Hz, 1H), 4.39-4.13 (m, 4H), 3.93 (d, J=13.5 Hz, 1H), 3.72-3.59 (m, 3H), 3.20 (t, J=13.0 Hz, 1H), 3.12-2.94 (m, 2H), 2.93-2.84 (m, 1H), 2.67 (t, J=12.9 Hz, 1H), 2.17-2.01 (m, 4H), 2.00-1.87 (m. 3H), 1.83-1.71 (m, 1H), 1.64-1.51 (m, 2H), 1.49-1.33 (m, 2H). MS m/z: 517.3 [M+H]⁺.

### 1-(4-(5-methyl-3-(3-(((2-(trifluoromethyl)pyridin-3-yl)oxy)methyl)piperidin-1-yl)-5H-pyrrolo[2,3-b]pyrazin-6-yl)piperidin-1-yl)ethan-1-one (73)

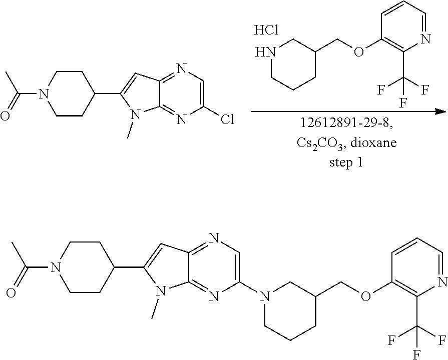 [SMILES: CC(=O)N1CCC(c2cc3ncc(Cl)nc3n2C)CC1 ; Cl ; FC(F)(F)c1ncccc1OCC1CCCNC1 ; CC(=O)N1CCC(c2cc3ncc(N4CCCC(COc5cccnc5C(F)(F)F)C4)nc3n2C)CC1]

To a stirred solution of 1-(4-(3-chloro-5-methyl-5H-pyrrolo[2,3-b]pyrazin-6-yl)piperidin-1-yl)ethan-1-one (50 mg, 0.171 mmol, 1 equiv) and 3-(piperidin-3-ylmethoxy)-2-(trifluoromethyl)pyridine hydrochloride (60.8 mg, 0.205 mmol, 1.2 equiv) in dioxane (1 mL) were added Cs₂CO₃ (111 mg, 0.342 mmol, 2 equiv) and 1612891-29-8 (14.3 mg, 0.017 mmol, 0.1 equiv) in portions at 25° C. The resulting mixture was stirred for 2 hours at 100° C. under nitrogen atmosphere. Desired product could be detected by LCMS. The reaction mixture was diluted by EtOAc (20 mL), washed by water (2×20 mL) and brine (1×20 mL), dried over anhydrous sodium sulfate. After filtration, the filtrate was concentrated. The residue was purified by reverse flash chromatography with the following conditions: column, C18 silica gel; mobile phase, MeCN in water, 5% to 100% gradient in 30 min; detector, UV 254 nm. Pure fractions were evaporated to dryness to afford 1-(4-(5-methyl-3-(3-(((2-(trifluoromethyl)pyridin-3-yl)oxy)methyl)piperidin-1-yl)-5H-pyrrolo[2,3-b]pyrazin-6-yl)piperidin-1-yl)ethan-1-one (20.6 mg, 23.1%) as a light yellow solid. ¹H NMR (400 MHZ, DMSO-d₆) δ 8.26 (d, J=4.3 Hz, 1H), 8.06 (s, 1H), 7.85-7.79 (m, 1H), 7.73-7.65 (m, 2H), 6.19 (s, 1H), 4.50 (t, J=12.6 Hz, 2H), 4.25-4.14 (m, 2H), 4.08 (t, J=8.9 Hz, 1H), 3.92 (d, J=13.4 Hz, 1H), 3.64 (s, 3H), 3.25-3.14 (m, 1H), 3.11-2.92 (m, 2H), 2.84-2.79 (m, 1H), 2.71-2.61 (m, 1H), 2.17-2.06 (m, 1H), 2.04 (s, 3H), 2.00-1.83 (m, 3H), 1.82-1.72 (m, 1H), 1.64-1.50 (m, 2H), 1.47-1.34 (m, 2H). MS m/z: 517.1 [M+H]⁺.

### 6-[(1R,5S,6S)-6-({[6-(trifluoromethyl)pyridin-2-yl]oxy}methyl)-3-azabicyclo[3.1.0]hexane-3-carbonyl]-1H-indole (74)

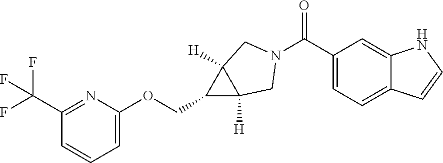 [SMILES: O=C(c1ccc2cc[nH]c2c1)N1C[C@@H]2[C@@H](COc3cccc(C(F)(F)F)n3)[C@@H]2C1]

Followed General Procedure E using (1R,5S,6S)-6-({[6-(trifluoromethyl)pyridin-2-yl]oxy}methyl)-3-azabicyclo[3.1.0]hexane hydrochloride (36.6 mg, 124 μmol) and 1H-indole-6-carboxylic acid (20 mg, 124 μmol) to afford 6-[(1R,5S,6S)-6-({[6-(trifluoromethyl)pyridin-2-yl]oxy}methyl)-3-azabicyclo[3.1.0]hexane-3-carbonyl]-1H-indole as a white foam (34 mg, 68%). ¹H NMR (500 MHZ, CDCl₃) δ 8.36 (s, 1H), 7.69 (t, J=7.8 Hz, 1H), 7.62 (d, J=8.1 Hz, 1H), 7.55 (s, 1H), 7.30 (t, J=2.9 Hz, 1H), 7.23 (d, J=7.3 Hz, 1H), 7.19 (dd, J=8.1, 1.3 Hz, 1H), 6.88 (d, J=8.4 Hz, 1H), 6.57 (dd, J=3.4, 1.9 Hz, 1H), 4.31 (d, J=12.1 Hz, 1H), 4.24 (d, J=7.2 Hz, 2H), 3.67 (s, 2H), 3.54 (d, J=12.3 Hz, 1H), 1.65 (d, J=34.1 Hz, 2H), 1.14 (tt, J=7.1, 3.3 Hz, 1H). MS m/z: 402.4 [M+H]⁺.

### (1R,5S,6S)-3-[3-(Propan-2-yl)-1H-pyrazole-5-carbonyl]-6-({[6-(trifluoromethyl)pyridin-2-yl]oxy}methyl)-3-azabicyclo[3.1.0]hexane (75)

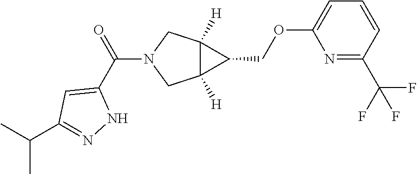 [SMILES: CC(C)c1cc(C(=O)N2C[C@@H]3[C@@H](COc4cccc(C(F)(F)F)n4)[C@@H]3C2)[nH]n1]

Followed General Procedure E using (1R,5S,6S)-6-({[6-(trifluoromethyl)pyridin-2-yl]oxy}methyl)-3-azabicyclo[3.1.0]hexane hydrochloride (36.6 mg, 124 μmol) and 3-(propan-2-yl)-1H-pyrazole-5-carboxylic acid (19.1 mg, 124 μmol) to afford (1R,5S,6S)-3-[3-(propan-2-yl)-1H-pyrazole-5-carbonyl]-6-({[6-(trifluoromethyl)pyridin-2-yl]oxy}methyl)-3-azabicyclo[3.1.0]hexane (22 mg, 45%). ¹H NMR (500 MHZ, CDCl₃) δ 10.24 (br s, 1H), 7.70 (q, J=6.9 Hz, 1H), 7.24 (s, 1H, overlapped with CDCl₃ solvent peak), 7.01-6.76 (m, 1H), 6.44 (d, J=4.4 Hz, 1H), 4.28 (h, J=10.9, 9.0 Hz, 2H), 4.21-4.07 (m, 2H), 3.87 (dd, J=11.4, 5.1 Hz, 1H), 3.62 (dd, J=12.6, 5.4 Hz, 1H), 3.01 (p, J=6.8 Hz, 1H), 1.76 (ddd, J=44.0, 7.7, 3.8 Hz, 2H), 1.24 (s, 6H), 1.11 (dd, J=7.3, 3.7 Hz, 1H). MS m/z: 395.4 [M+H]⁺.

### 3-(phenoxymethyl)-1-[5-(3-phenyloxolan-3-yl)-1,3,4-oxadiazole-2-carbonyl]piperidine (76)

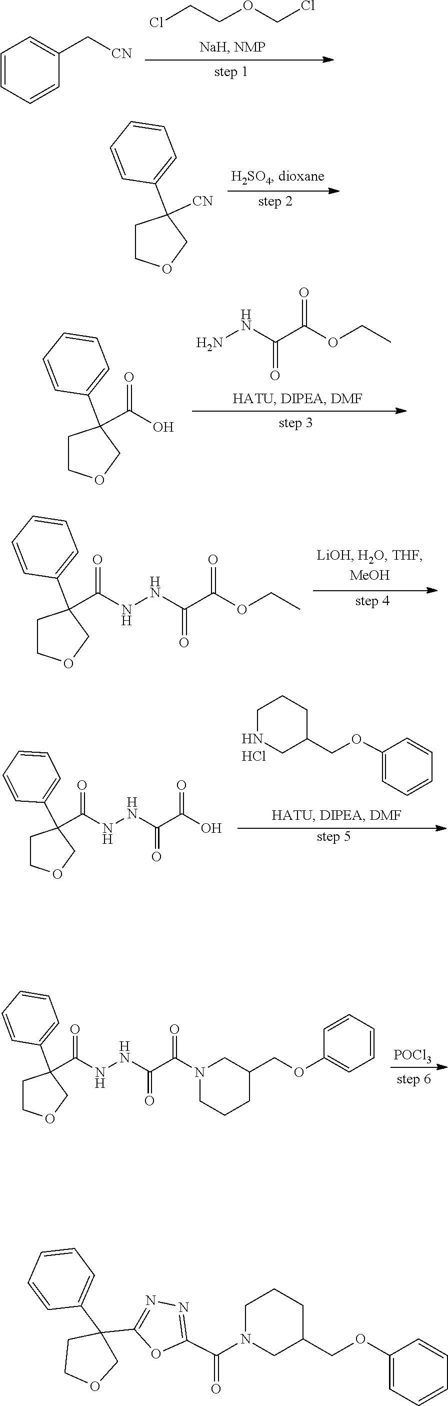

Step 1: 3-phenyltetrahydrofuran-3-carbonitrile: To a stirred mixture of NaH (2.05 g, 51.2 mmol, 3.00 equiv) in NMP (20 mL) was added a solution of 2-phenylacetonitrile (2.00 g, 17.1 mmol, 1.00 equiv) and 1-chloro-2-(chloromethoxy)ethane (2.20 g, 17.1 mmol, 1.00 equiv) in Et₂O (5 mL) dropwise at −20° C. The resulting mixture was stirred for overnight at room temperature. The reaction was quenched with sat. NH₄Cl (aq.) at 0° C. The resulting mixture was extracted with EtOAc (3×15 mL). The combined organic layers were washed with brine (2×20 mL), dried over anhydrous Na₂SO₄. After filtration, the filtrate was concentrated under reduced pressure. The residue was purified by reverse flash with the following conditions: column, C18 silica; mobile phase, MeCN in water, 5% to 95% gradient in 20 min; detector, UV 200 nm. This provided 3-phenyltetrahydrofuran-3-carbonitrile (1.25 g. 42.3%) as a light brown oil. MS m/z: 174 [M+H]⁺.

Step 2: 3-phenyltetrahydrofuran-3-carboxylic acid: To a stirred solution of 3-phenyltetrahydrofuran-3-carbonitrile (1.15 g, 6.64 mmol, 1.00 equiv) in dioxane (4.60 mL) was added H₂SO₄ (6.9 mL, 62.1 mmol, 9.36 equiv) dropwise at 0° C. The resulting mixture was stirred for overnight at 110° C. The mixture was allowed to cool down to room temperature and extracted with EtOAc (3×15 mL). The organic layer was washed with 3×20 mL of NaOH (2 N). The aqueous layer was acidified to pH 5 with conc. HCl and extracted with EtOAc (3×25 mL). The combined organic layers were washed with brine (2×20 mL), dried over anhydrous Na₂SO₄. After filtration, the filtrate was concentrated under reduced pressure. This provided 3-phenyltetrahydrofuran-3-carboxylic acid (620 mg, 48.6%) as a light brown solid. MS m/z: 193 [M+H]⁺.

Step 3: ethyl 2-oxo-2-(2-(3-phenyltetrahydrofuran-3-carbonyl)hydrazineyl)acetate: To a stirred mixture of 3-phenyltetrahydrofuran-3-carboxylic acid (620 mg, 3.23 mmol, 1.00 equiv) and ethyl 2-hydrazineyl-2-oxoacetate (16.50 mg, 0.125 mmol, 1.20 equiv) in DMF (5 mL) were added HATU (1.35 g, 3.55 mmol, 1.10 equiv) and DIPEA (1.25 g, 9.68 mmol, 3.00 equiv) at 0° C. The resulting mixture was stirred for 2 h at room temperature. The resulting mixture was diluted with water (20 mL). The resulting mixture was extracted with EtOAc (3×25 mL). The combined organic layers were washed with brine (2×30 mL), dried over anhydrous Na₂SO₄. After filtration, the filtrate was concentrated under reduced pressure. The residue was purified by reverse flash with the following conditions: column, C18 gel; mobile phase, MeCN in water (0.1% FA), 0% to 50% gradient in 20 min; detector, UV 200 nm. This provided ethyl 2-oxo-2-(2-(3-phenyltetrahydrofuran-3-carbonyl)hydrazineyl)acetate (640 mg, 64.8%) as a light yellow semi-solid. MS m/z: 307 [M+H]⁺.

Step 4: 2-oxo-2-(2-(3-phenyltetrahydrofuran-3-carbonyl)hydrazineyl)acetic acid: To a stirred solution of ethyl 2-oxo-2-(2-(3-phenyltetrahydrofuran-3-carbonyl)hydrazineyl)acetate (350 mg, 1.14 mmol, 1.00 equiv) in THF (3 mL)/MeOH (6 mL) was added a solution of LiOH·H₂O (57.5 mg, 1.37 mmol, 1.20 equiv) in H₂O (3 mL) at 0° C.

The resulting mixture was stirred for 6 h at room temperature. The mixture was acidified to pH 5 with HCl (aq.) (2N). The residue was purified by reverse flash with the following conditions: column, C18 gel; mobile phase, MeCN in water (0.1% FA), 0% to 30% gradient in 20 min; detector, UV 200 nm. This provided 2-oxo-2-(2-(3-phenyltetrahydrofuran-3-carbonyl)hydrazineyl)acetic acid (190 mg, 59.8%) as an off-white solid. MS m/z: 279 [M+H]⁺.

Step 5: N′-(2-oxo-2-(3-(phenoxymethyl)piperidin-1-yl)acetyl)-3-phenyltetrahydrofuran-3-carbohydrazide: To a stirred mixture of 2-oxo-2-(2-(3-phenyltetrahydrofuran-3-carbonyl)hydrazineyl)acetic acid (170 mg, 0.611 mmol, 1.00 equiv) and 3-(phenoxymethyl)piperidine (153 mg, 0.672 mmol, 1.10 equiv) in DMF (4 mL) were added HATU (256 mg, 0.672 mmol, 1.10 equiv) and DIPEA (237 mg, 1.83 mmol, 3.00 equiv) at 0° C. The resulting mixture was stirred for 2 h at room temperature. The resulting mixture was diluted with water (15 mL). The resulting mixture was extracted with EtOAc (3×10 mL). The combined organic layers were washed with brine (2×15 mL), dried over anhydrous Na₂SO₄. After filtration, the filtrate was concentrated under reduced pressure. The residue was purified by reverse flash with the following conditions: column, C18 gel; mobile phase, MeCN in water (0.1% FA), 5% to 50% gradient in 20 min; detector, UV 254 nm. This provided N′-(2-oxo-2-(3-(phenoxymethyl)piperidin-1-yl)acetyl)-3-phenyltetrahydrofuran-3-carbohydrazide (150 mg, 54.4%) as an off-white solid. MS m/z: 452 [M+H]⁺.

Step 6: (3-(phenoxymethyl)piperidin-1-yl)(5-(3-phenyltetrahydrofuran-3-yl)-1,3,4-oxadiazol-2-yl)methanone: A solution of N′-(2-oxo-2-(3-(phenoxymethyl)piperidin-1-yl)acetyl)-3-phenyltetrahydrofuran-3-carbohydrazide (110 mg, 0.244 mmol, 1.00 equiv) in POCl₃ (3 mL) was stirred for 6 h at 100° C. under nitrogen atmosphere. The reaction was quenched with sat. NaHCO₃(aq.) at 0° C. The resulting mixture was extracted with EtOAc (3×15 mL). The combined organic layers were dried over anhydrous Na₂SO₄. After filtration, the filtrate was concentrated under reduced pressure. The residue was purified by Prep-TLC (PE/EA 1:2) to afford the crude product. The crude product was purified by reverse flash with the following conditions: column, C18 gel; mobile phase, MeCN in water (0.1% FA), 5% to 50% gradient in 20 min; detector, UV 220 nm. This provided (3-(phenoxymethyl)piperidin-1-yl)(5-(3-phenyltetrahydrofuran-3-yl)-1,3,4-oxadiazol-2-yl)methanone (15.5 mg, 14.6%) as an off-white solid. ¹H NMR (300 MHZ, CDCl₃): δ 7.42-7.29 (m, 6H), 7.28-7.25 (m, 1H), 7.06-6.78 (m, 3H), 4.91-4.78 (m, 1H), 4.76-4.66 (m, 1H), 4.51-4.06 (m, 3H), 3.98-3.79 (m, 2H), 3.44-3.20 (m, 2H), 3.13-2.84 (m, 1H), 2.68-2.52 (m, 1H), 2.22-2.06 (m, 1H), 2.06-1.96 (m, 1H), 1.96-1.83 (m, 1H), 1.76-1.64 (m, 2H), 1.61-1.50 (m, 1H). MS, m/z: 434.1 [M+H]⁺.

### 1-(2,2-Difluoroethyl)-6-((2S,5R)-2-methyl-5-(((2-(trifluoromethyl)pyridin-3-yl)oxy)methyl)piperidin-1-yl)-1H-pyrazolo[3,4-b]pyrazine (77)

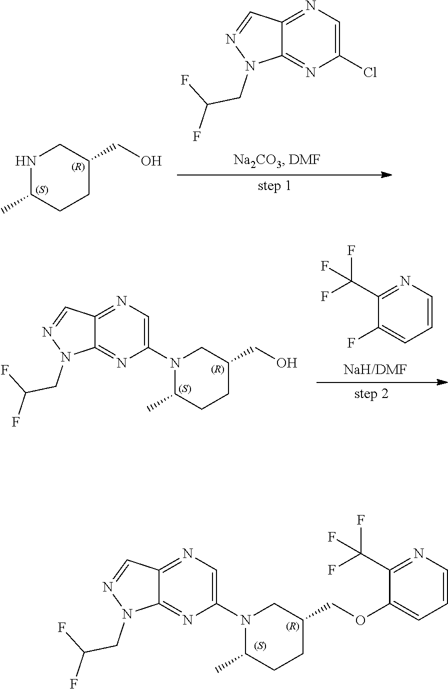

Step 1: ((3R,6S)-1-(1-(2,2-difluoroethyl)-1H-pyrazolo[3,4-b]pyrazin-6-yl)-6-methylpiperidin-3-yl)methanol: To a stirred solution of ((3R,6S)-6-methylpiperidin-3-yl)methanol (150 mg, 1.16 mmol, 1.00 equiv) and 6-chloro-1-(2,2-difluoroethyl)pyrazolo[3,4-b]pyrazine (253 mg, 1.16 mmol, 1 equiv) in DMF (2 mL) was added Na₂CO₃ (307 mg, 2.90 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided ((3R,6S)-1-(1-(2,2-difluoroethyl)-1H-pyrazolo[3,4-b]pyrazin-6-yl)-6-methylpiperidin-3-yl)methanol (220 mg, 60.8%) as a yellow oil. MS m/z: 312 [M+H]⁺.

Step 2: 1-(2,2-difluoroethyl)-6-((2S,5R)-2-methyl-5-(((2-(trifluoromethyl)pyridin-3-yl)oxy)methyl)piperidin-1-yl)-1H-pyrazolo[3,4-b]pyrazine: To a stirred solution of ((3R,6S)-1-(1-(2,2-difluoroethyl)-1H-pyrazolo[3,4-b]pyrazin-6-yl)-6-methylpiperidin-3-yl)methanol (200 mg, 0.642 mmol, 1.00 equiv) and 3-fluoro-2-(trifluoromethyl)pyridine (212 mg. 1.28 mmol, 2 equiv) in DMF (2 mL) was added NaH (18.5 mg, 0.770 mmol, 1.2 equiv) in portions at 0° C. The resulting mixture was stirred for 3 h at room temperature. The reaction was quenched by the addition of sat. NH₄Cl (aq.) (5 mL) at 0° C. The resulting mixture was extracted with EtOAc (3×10 mL). The combined organic layers were washed with water (10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 3-1-(2,2-difluoroethyl)-6-((2S,5R)-2-methyl-5-(((2-(trifluoromethyl)pyridin-3-yl)oxy)methyl)piperidin-1-yl)-1H-pyrazolo[3,4-b]pyrazine (100 mg, 34.1%) as a white solid. ¹H NMR (300 MHz, DMSO-d₆) δ 8.43 (s, 1H), 8.29-8.28 (m, 1H), 8.13 (s, 1H), 7.86-7.83 (m, 1H), 7.74-7.70 (m, 1H), 6.61-6.23 (m, 1H), 4.87 (s, 1H), 4.82-4.60 (m, 3H), 4.32-4.27 (m, 1H), 4.15-4.19 (m, 1H), 2.95-2.86 (m, 1H), 2.07 (s, 1H), 1.90-1.59 (m, 4H), 1.24-1.22 (m, 3H). MS m/z: 457.2 [M+H]⁺.

### 3-{[(3S,6S)-1-[1-(2,2-Difluoroethyl)pyrazolo[3,4-b]pyrazin-6-yl]-6-methylpiperidin-3-yl]methoxy}-2-(trifluoromethyl)pyridine (78)

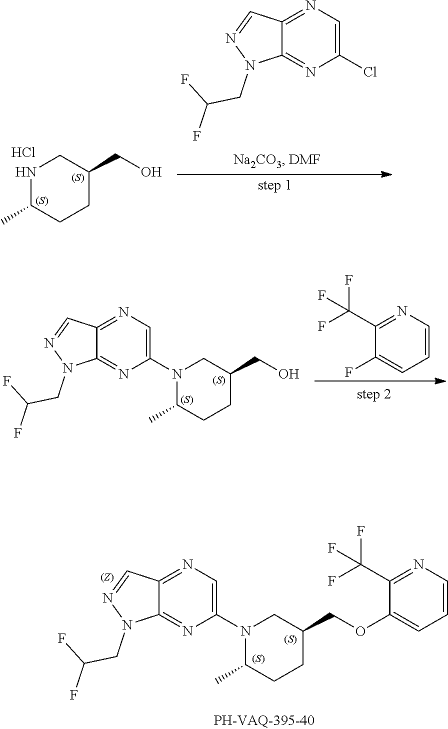

Step 1: ((3S,6S)-1-(1-(2,2-difluoroethyl)-1H-pyrazolo[3,4-b]pyrazin-6-yl)-6-methylpiperidin-3-yl)methanol: To a stirred solution of ((3S,6S)-6-methylpiperidin-3-yl)methanol hydrochloride (200 mg, 1.21 mmol, 1 equiv) and 6-chloro-1-(2,2-difluoroethyl)-1H-pyrazolo[3,4-b]pyrazine (264 mg, 1.21 mmol, 1 equiv) in DMF (3 mL) was added Na₂CO₃ (312 mg, 3.02 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided ((3S,6S)-1-(1-(2,2-difluoroethyl)-1H-pyrazolo[3,4-b]pyrazin-6-yl)-6-methylpiperidin-3-yl)methanol (120 mg, 31.93%) as a yellow oil. MS m/z: 312 [M+H]⁺.

Step 2: 1-(2,2-difluoroethyl)-6-((2S,5S)-2-methyl-5-(((2-(trifluoromethyl)pyridin-3-yl)oxy)methyl)piperidin-1-yl)-1H-pyrazolo[3,4-b]pyrazine: To a stirred solution of ((3S,6S)-1-(1-(2,2-difluoroethyl)-1H-pyrazolo[3,4-b]pyrazin-6-yl)-6-methylpiperidin-3-yl)methanol (120 mg, 0.385 mmol, 1 equiv) and 3-fluoro-2-(trifluoromethyl)pyridine (69.99 mg, 0.424 mmol, 1.1 equiv) in DMF (2 mL) was added NaH (13.9 mg, 0.578 mmol, 1.5 equiv) in portions at 0° C. The resulting mixture was stirred for 3 h at room temperature. The reaction was quenched by the addition of sat. NH₄Cl (aq.) (5 mL) at 0° C. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 1-(2,2-difluoroethyl)-6-((2S,5S)-2-methyl-5-(((2-(trifluoromethyl)pyridin-3-yl)oxy)methyl)piperidin-1-yl)-1H-pyrazolo[3,4-b]pyrazine (73.4 mg, 41.0%) as an off-white semi-solid. ¹H NMR (400 MHZ, DMSO-d₆) δ 8.29 (d, J=1.2 Hz, 1H), 8.22 (d, J=4.4 Hz, 1H), 8.07 (d, J=1.2 Hz, 1H), 7.74 (d, J=8.6 Hz, 1H), 7.64-7.57 (m, 1H), 6.47-6.14 (m. 1H), 4.67 (t, J=6.4 Hz, 1H), 4.55-4.45 (m, 3H), 4.25-4.08 (m, 2H), 3.36 (d, J=4.4 Hz, 1H), 2.37 (s, 1H), 2.06-1.90 (m, 2H), 1.52 (d, J=10.8 Hz, 2H), 1.25 (d, J=6.5 Hz, 3H). MS m/z: 457.0 [M+H]⁺.

### 1-(2,2-Difluoroethyl)-6-((2R,5R)-2-methyl-5-(((2-(trifluoromethyl)pyridin-3-yl)oxy)methyl)piperidin-1-yl)-1H-pyrazolo[3,4-b]pyrazine (79)

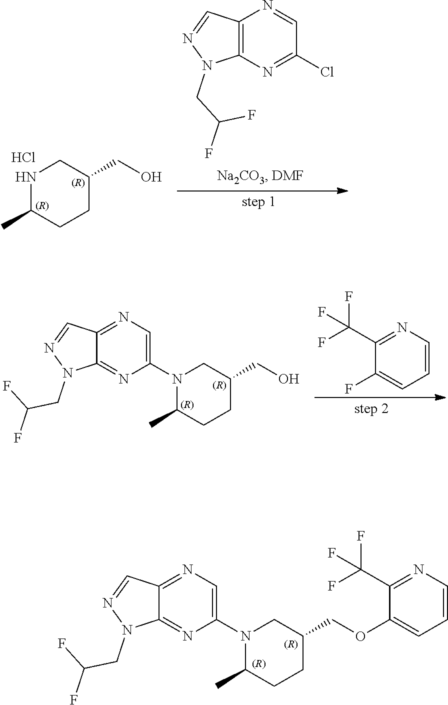

Step 1: ((3R,6R)-1-(1-(2,2-difluoroethyl)-1H-pyrazolo[3,4-b]pyrazin-6-yl)-6-methylpiperidin-3-yl)methanol: To a stirred solution of ((3R,6R)-6-methylpiperidin-3-yl)methanol hydrochloride (150 mg, 1.16 mmol, 1.00 equiv) and 6-chloro-1-(2,2-difluoroethyl)-1H-pyrazolo[3,4-b]pyrazine (253 mg, 1.16 mmol, 1 equiv) in DMF (2 mL) was added Na₂CO₃ (307 mg, 2.90 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided ((3R,6R)-1-(1-(2,2-difluoroethyl)-1H-pyrazolo[3,4-b]pyrazin-6-yl)-6-methylpiperidin-3-yl)methanol (90 mg, 31.9%) as a yellow oil. MS m/z: 312 [M+H]⁺.

Step 2: 1-(2,2-difluoroethyl)-6-((2R,5R)-2-methyl-5-(((2-(trifluoromethyl)pyridin-3-yl)oxy)methyl)piperidin-1-yl)-1H-pyrazolo[3,4-b]pyrazine: To a stirred solution of ((3R,6R)-1-(1-(2,2-difluoroethyl)-1H-pyrazolo[3,4-b]pyrazin-6-yl)-6-methylpiperidin-3-yl)methanol (80 mg, 0.257 mmol, 1 equiv) and 3-fluoro-2-(trifluoromethyl)pyridine (46.6 mg, 0.283 mmol, 1.1 equiv) in DMF (1 mL) was added NaH (15.4 mg, 0.386 mmol, 1.5 equiv) at 0° C. The resulting mixture was stirred for 1 h at room temperature. The reaction was quenched by the addition of sat. NH₄Cl (aq.) (5 mL) at 0° C. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 1-(2,2-difluoroethyl)-6-((2R,5R)-2-methyl-5-(((2-(trifluoromethyl)pyridin-3-yl)oxy)methyl)piperidin-1-yl)-1H-pyrazolo[3,4-b]pyrazine (63 mg, 53.4%) as a white solid. ¹H NMR (300 MHZ, CD₃OD) δ 8.24-8.15 (m, 2H) 7.94 (s, 1H), 7.67-7.58 (m, 1H), 7.53-7.50 (m, 1H), 6.35-5.93 (m, 1H), 4.79-4.63 (m, 2H), 4.57-4.44 (m, 2H), 4.25-4.17 (m, 1H), 4.15-4.08 (m, 1H), 3.48-3.40 (m, 1H), 2.54-2.44 (m, 1H), 2.23-1.96 (m, 2H), 1.70-1.56 (m, 2H), 1.39-1.35 (m, 3H). MS m/z: 457.1 [M+H]⁺.

### 1-(2,2-Difluoroethyl)-6-((2R,5S)-2-methyl-5-(((2-(trifluoromethyl)pyridin-3-yl)oxy)methyl)piperidin-1-yl)-1H-pyrazolo[3,4-b]pyrazine (80)

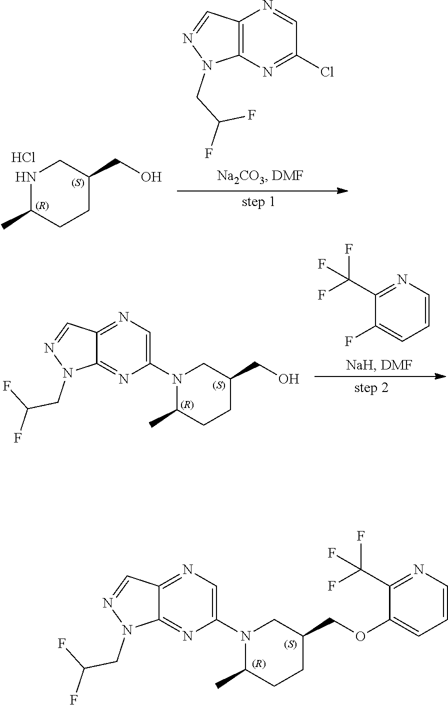

Step 1: ((3S,6R)-1-(1-(2,2-difluoroethyl)-1H-pyrazolo[3,4-b]pyrazin-6-yl)-6-methylpiperidin-3-yl)methanol: A solution of ((3S,6R)-6-methylpiperidin-3-yl)methanol hydrochloride (150 mg, 1.16 mmol, 1 equiv), Na₂CO₃ (246 mg, 2.32 mmol, 2 equiv) and 6-chloro-1-(2,2-difluoroethyl)-1H-pyrazolo[3,4-b]pyrazine (279 mg, 1.27 mmol, 1.1 equiv) in DMF (3 mL) was stirred for 2 h at 90° C. The residue was purified by reverse flash chromatography with the following conditions: column, C18 silica gel; mobile phase, MeCN in water, 10% to 100 gradient in 30 min; detector, UV 254 nm. This provided ((3S,6R)-1-(1-(2,2-difluoroethyl)-1H-pyrazolo[3,4-b]pyrazin-6-yl)-6-methylpiperidin-3-yl)methanol (150 mg, 41.5%) as a colorless oil. MS m/z: 312 [M+H]⁺.

Step 2: 1-(2,2-difluoroethyl)-6-((2R,5S)-2-methyl-5-(((2-(trifluoromethyl)pyridin-3-yl)oxy)methyl)piperidin-1-yl)-1H-pyrazolo[3,4-b]pyrazine: A solution of ((3S,6R)-1-(1-(2,2-difluoroethyl)-1H-pyrazolo[3,4-b]pyrazin-6-yl)-6-methylpiperidin-3-yl)methanol (100 mg, 0.321 mmol, 1 equiv) and 3-fluoro-2-(trifluoromethyl)pyridine (63.6 mg, 0.385 mmol, 1.2 equiv) in DMF (3 mL) was added NaH (9.2 mg, 0.385 mmol, 1.2 equiv) in portions at 0° C. The resulting mixture was stirred for 2 h at room temperature. The reaction was quenched by the addition of sat. NH₄Cl (aq.) (5 mL) at 0° C. The resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by reverse flash chromatography with the following conditions: column, silica gel; mobile phase, MeCN in water, 10% to 100% gradient in 10 min; detector, UV 254 nm. This provided 1-(2,2-difluoroethyl)-6-((2R,5S)-2-methyl-5-(((2-(trifluoromethyl)pyridin-3-yl)oxy)methyl)piperidin-1-yl)-1H-pyrazolo[3,4-b]pyrazine (60 mg, 40.5%) as a white solid. ¹H NMR (400 MHz, DMSO-d₆) δ 8.42 (s, 1H), 8.28 (dd, J=4.5, 1.1 Hz, 1H), 8.13 (s, 1H), 7.84 (d, J=8.5 Hz, 1H), 7.72 (dd, J=8.6, 4.5 Hz, 1H), 6.41 (tt, J=55.0, 4.0 Hz, 1H), 4.93-4.82 (m, 1H), 4.79-4.58 (m, 3H), 4.29 (dd, J=9.5, 4.6 Hz, 1H), 4.15-4.08 (m, 1H), 2.90 (t, J=12.6 Hz, 1H), 2.13-2.00 (m, 1H), 1.89-1.63 (m, 4H), 1.22 (d, J=6.8 Hz, 3H). MS m/z: 456.9 [M+H]⁺.

### (5-Methyl-6-phenyl-5H-pyrrolo[2,3-b]pyrazin-7-yl)(3-((o-tolyloxy)methyl)piperidin-1-yl)methanone (81)

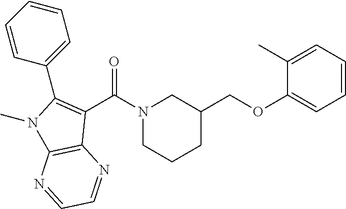 [SMILES: Cc1ccccc1OCC1CCCN(C(=O)c2c(-c3ccccc3)n(C)c3nccnc23)C1]

Followed General Procedure E using 3-(2-methylphenoxymethyl)piperidine (34.1 mg, 0.166 mmol, 1.2 equiv) to afford (5-methyl-6-phenyl-5H-pyrrolo[2,3-b]pyrazin-7-yl)(3-((o-tolyloxy)methyl)piperidin-1-yl)methanone (58.3 mg, 95.0%) as a white solid. ¹H NMR (400 MHZ, DMSO-d₆) δ 8.64-8.24 (m, 2H), 7.66-7.45 (m, 5H), 7.19-6.93 (m, 2H), 6.92-6.68 (m, 2H), 4.39 (dd, J=30.5, 12.6 Hz, 1H), 3.93-3.71 (m, 5H), 3.51 (t, J=9.3 Hz, 1H), 3.00-2.74 (m, 2H), 2.17 (s, 1H), 1.93-1.56 (m, 3H), 1.52-1.21 (m, 4H). MS m/z: 441.3 [M+H]⁺.

### (5-Methyl-6-phenyl-5H-pyrrolo[2,3-b]pyrazin-7-yl)(3-(((2-(trifluoromethyl)pyridin-3-yl)oxy)methyl)piperidin-1-yl)methanone (82)

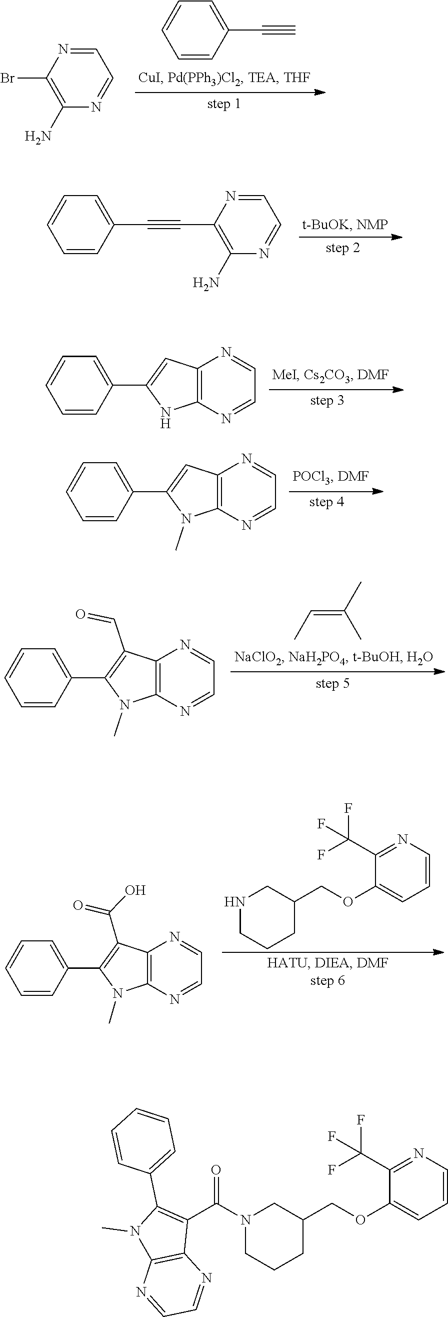

Step 1: 3-(phenylethynyl)pyrazin-2-amine: To the solution of 3-bromopyrazin-2-amine (1.00 g, 5.75 mmol, 1.00 equiv) and ethynylbenzene (0.700 g, 6.90 mmol, 1.20 equiv) and CuI (0.110 g, 0.575 mmol, 0.1 equiv) and TEA (1.74 g, 17.3 mmol, 3.00 equiv) in THF (10 mL) was added Pd(PPh₃)₂Cl₂ (0.400 g. 0.575 mmol, 0.1 equiv) under N₂ atmosphere. The resulting mixture was stirred for 2 h at 80° C. The residue was purified by silica gel column chromatography, eluted with PE/EtOAc (5:1) to afford 3-(phenylethynyl)pyrazin-2-amine (480 mg, 96.9%) as a white solid. The product was confirmed by TLC. MS m/z: 196 [M+H]⁺.

Step 2: 6-phenyl-5H-pyrrolo[2,3-b]pyrazine: To a stirred solution of 3-(phenylethynyl)pyrazin-2-amine (200 mg, 1.03 mmol, 1.00 equiv) in (2 mL) was added t-BuOK (230 mg, 2.05 mmol, 2.00 equiv). The resulting mixture was stirred at 80° C. for 2 h under N₂ atmosphere. The residue was purified by reverse flash chromatography with the following conditions: column, C18 silica gel; mobile phase phase, MeCN in water (0.1% FA), 5% to 100% gradient in 20 min; detector, UV 254/220 nm. This provided 6-phenyl-5H-pyrrolo[2,3-b]pyrazine (190 mg, 95.0%) as a yellow oil. MS m/z: 196 [M+H]⁺.

Step 3: 5-methyl-6-phenyl-5H-pyrrolo[2,3-b]pyrazine: To a stirred solution of 6-phenyl-5H-pyrrolo[2,3-b]pyrazine (950 mg, 4.87 mmol, 1.00 equiv) and CH₃I (1.39 g, 7.30 mmol, 1.50 equiv) in THF (10 mL) was added Cs₂CO₃ (2.38 g. 7.30 mmol, 1.50 equiv). The resulting mixture was stirred for 2 h at room temperature under N₂ atmosphere. The residue was purified by silica gel column chromatography, eluted with PE/EtOAc (6:1) to afford 5-methyl-6-phenyl-5H-pyrrolo[2,3-b]pyrazine (490 mg, 48.1%) as a yellow solid. MS m/z: 210 [M+H]⁺.

Step 4: 5-methyl-6-phenyl-5H-pyrrolo[2,3-b]pyrazine-7-carbaldehyde: To a stirred solution of POCl₃ (989 mg, 6.45 mmol, 3.00 equiv) in DMF (7 mL) was added 5-methyl-6-phenylpyrrolo[2,3-b]pyrazine (450 mg, 2.151 mmol, 1.00 equiv). The resulting mixture was stirred for 3 hours at 0° C. The reaction was monitored by LCMS. The mixture was diluted by water (20 mL) neutralized to PH ˜8 with saturated NaHCO₃(aq.). The resulting mixture was extracted with EtOAc (3×10 mL). The combined organic layers were washed with water (2×20 mL) and brine (20 mL), dried over anhydrous Na₂SO₄. After filtration, the filtrate was concentrated under reduced pressure. This provided 5-methyl-6-phenylpyrrolo[2,3-b]pyrazine-7-carbaldehyde (200 mg, 39.2%) as a white solid. MS m/z: 238 [M+H]⁺.

Step 5: 5-methyl-6-phenyl-5H-pyrrolo[2,3-b]pyrazine-7-carboxylic acid: To a stirred mixture of 5-methyl-6-phenylpyrrolo[2,3-b]pyrazine-7-carbaldehyde (200 mg, 0.843 mmol, 1.00 equiv) in H₂O (1.00 mL) was added NaH₂PO₄ (606 mg, 5.06 mmol, 6 equiv) at 0° C. under air atmosphere. After 5 minutes, t-BuOH (5.00 mL), 2-methyl-2-butene (147.80 mg, 2.107 mmol, 2.5 equiv) and NaClO₂ (73.90 mg, 1.264 mmol, 1.5 equiv) were added. After 16 hours, reaction mixture was diluted with ethyl acetate (20 mL), washed with water (2×15 mL) and brine (20 mL), then dried over magnesium sulfate, filtered, and concentrated to give 5-methyl-6-phenylpyrrolo[2,3-b]pyrazine-7-carboxylic acid (80 mg, 37.47%) as a white solid which was used without further purification. MS m/z: 268 [M+H]⁺.

Step 6: (5-methyl-6-phenyl-5H-pyrrolo[2,3-b]pyrazin-7-yl)(3-(((2-(trifluoromethyl)pyridin-3-yl)oxy)methyl)piperidin-1-yl)methanone: To a stirred solution of 5-methyl-6-phenylpyrrolo[2,3-b]pyrazine-7-carboxylic acid (50.0 mg, 0.197 mmol, 1.00 equiv) and HATU (90.1 mg, 0.236 mmol, 1.20 equiv) in DMF (1.5 mL) were added 3-(piperidin-3-ylmethoxy)-2-(trifluoromethyl)pyridine (56.5 mg, 0.217 mmol, 1.10 equiv) and DIEA (76.6 mg, 0.591 mmol, 3.00 equiv). The resulting mixture was stirred for 2 h at room temperature under N₂ atmosphere. The residue was purified by reverse flash chromatography with the following conditions: column, C18 silica gel; mobile phase, ACN in water, 5% to 100% gradient in 20 min; detector, UV 254 nm. This provided (5-methyl-6-phenyl-5H-pyrrolo[2,3-b]pyrazin-7-yl)(3-(((2-(trifluoromethyl)pyridin-3-yl)oxy)methyl)piperidin-1-yl)methanone (43 mg 43.0%) as a white solid. ¹H NMR (400 MHz, DMSO-d₆) δ 8.54-8.10 (m, 3H), 7.78-7.49 (m, 7H), 4.45-4.26 (m, 1H), 4.14-3.94 (m, 1H), 3.74 (s, 5H), 3.00-2.71 (m, 2H), 1.96-1.53 (m, 3H), 1.46-1.19 (m, 2H). MS m/z: 496.2 [M+H]⁺.

### (6-(4-Fluorophenyl)-5-methyl-5H-pyrrolo[2,3-b]pyrazin-7-yl)(3-(((2-(trifluoromethyl)pyridin-3-yl)oxy)methyl)piperidin-1-yl)methanone (83)

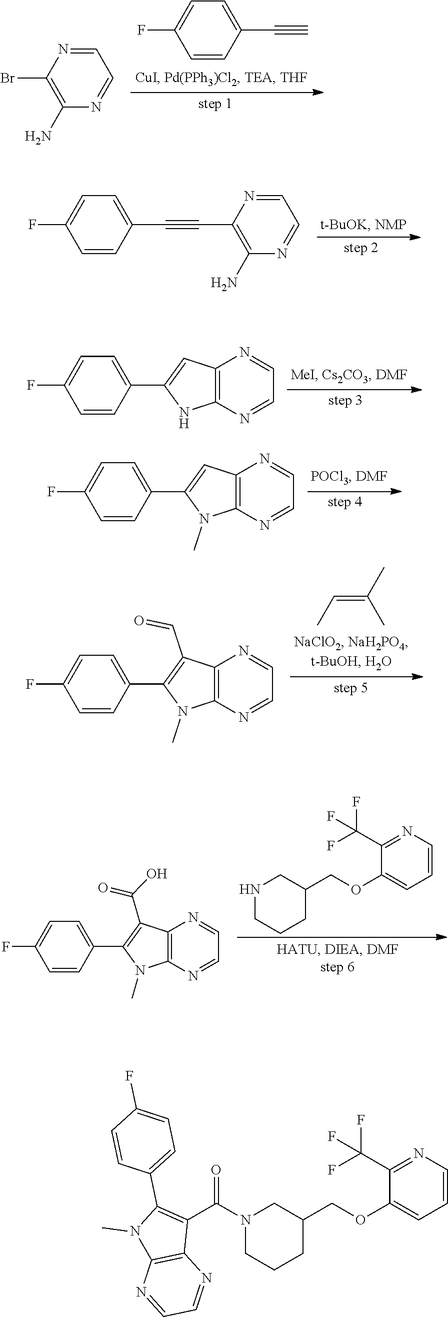

Step 1: 3-((4-fluorophenyl)ethynyl)pyrazin-2-amine: To a stirred solution of 1-ethynyl-4-fluorobenzene (829 mg, 6.90 mmol, 1.20 equiv) and 3-bromopyrazin-2-amine (1.00 g. 5.75 mmol, 1.00 equiv) in THF (20 mL) were added CuI (110 mg, 0.575 mmol, 0.100 equiv) and Pd(PPh₃)₂Cl₂ (404 mg, 0.575 mmol, 0.100 equiv) and TEA (1.75 g, 17.3 mmol, 3.00 equiv) under N₂ atmosphere. The final reaction mixture was irradiated with microwave radiation for 2 h at 80° C. The mixture was allowed to cool down to room temperature. The residue was purified by silica gel column chromatography, eluted with PE/EtOAc (1:1) to afford 3-[2-(4-fluorophenyl)ethynyl]pyrazin-2-amine (980 mg, 78.0%) as a red oil. The product was confirmed by TLC. MS m/z: 214 [M+H]⁺.

Step 2: 6-(4-fluorophenyl)-5H-pyrrolo[2,3-b]pyrazine: To a stirred solution of 3-((4-fluorophenyl)ethynyl)pyrazin-2-amine (900 mg, 4.22 mmol, 1.00 equiv) and t-BuOK (948 mg, 8.44 mmol, 2.00 equiv). The resulting mixture was stirred at 80° C. for 2 h under N₂ atmosphere. The residue was purified by reverse flash chromatography with the following conditions: column, C18 silica gel; mobile phase phase, MeCN in water (0.1% FA), 5% to 100% gradient in 20 min; detector, UV 254/220 nm. This provided 6-(4-fluorophenyl)-5H-pyrrolo[2,3-b]pyrazine (850 mg, 94.5%) as a red oil. MS m/z: 214 [M+H]⁺.

Step 3: 6-(4-fluorophenyl)-5-methyl-5H-pyrrolo[2,3-b]pyrazine: To a stirred solution/mixture of 6-(4-fluorophenyl)-5H-pyrrolo[2,3-b]pyrazine (850 mg, 3.99 mmol, 1.00 equiv) and CH₃I (849 mg, 5.98 mmol, 1.50 equiv) in THF (20 mL) was added Cs₂CO₃ (1.95 g. 5.98 mmol, 1.50 equiv). The resulting mixture was stirred for 2 h at room temperature under N₂ atmosphere. The residue was purified by silica gel column chromatography, eluted with PE/EtOAc (6:1) to afford 6-(4-fluorophenyl)-5-methylpyrrolo[2,3-b]pyrazine (450 mg, 49.7%) as a red oil. The product was confirmed by TLC. MS m/z: 228 [M+H]⁺.

Step 4: 6-(4-fluorophenyl)-5-methyl-5H-pyrrolo[2,3-b]pyrazine-7-carbaldehyde: To a stirred solution of POCl₃ (911 mg, 5.940 mmol, 3.00 equiv) in DMF (7 mL) was added 6-(4-fluorophenyl)-5-methylpyrrolo[2,3-b]pyrazine (450 mg, 2.151 mmol, 1.00 equiv). The resulting mixture was stirred for 3 hours at 0° C. The reaction was monitored by LCMS. The mixture was diluted by water (20 mL) neutralized to pH ˜8 with saturated NaHCO₃(aq.). The resulting mixture was extracted with EtOAc (3×10 mL). The combined organic layers were washed with water (2×20 mL) and brine (20 mL), dried over anhydrous Na₂SO₄. After filtration, the filtrate was concentrated under reduced pressure. This provided 6-(4-fluorophenyl)-5-methyl-5H-pyrrolo[2,3-b]pyrazine-7-carbaldehyde (200 mg, 39.6%) as a white solid. MS m/z: 256 [M+H]⁺.

Step 5: 6-(4-fluorophenyl)-5-methyl-5H-pyrrolo[2,3-b]pyrazine-7-carboxylic acid: To a stirred mixture of 6-(4-fluorophenyl)-5-methylpyrrolo[2,3-b]pyrazine-7-carbaldehyde (80.0 mg, 0.313 mmol, 1.00 equiv) in H₂O (1.00 mL) was added NaH₂PO+(226 mg, 1.88 mmol, 6.00 equiv) at 0° C. under air atmosphere. After 5 minutes, t-BuOH (5.00 mL), 2-methyl-2-butene (55.0 mg, 0.782 mmol, 2.50 equiv) and NaClO₂ (42.5 mg, 0.470 mmol, 1.50 equiv) were added. After 16 hours, reaction mixture was diluted with ethyl acetate (20 mL), washed with water (2×15 mL) and brine (20 mL), then dried over magnesium sulfate, filtered, and concentrated to give 6-(4-fluorophenyl)-5-methylpyrrolo[2,3-b]pyrazine-7-carboxylic acid (40 mg, 47.05%) as a white solid. The product was confirmed by TLC. MS m/z: 272 [M+H]⁺.

Step 6: (6-(4-fluorophenyl)-5-methyl-5H-pyrrolo[2,3-b]pyrazin-7-yl)(3-(((2-(trifluoromethyl)pyridin-3-yl)oxy)methyl)piperidin-1-yl)methanone: To a stirred solution/mixture of 3-(piperidin-3-ylmethoxy)-2-(trifluoromethyl)pyridine (31.7 mg, 0.122 mmol, 1.10 equiv) and HATU (50.5 mg, 0.133 mmol, 1.20 equiv) in DMF (1 mL) were added 6-(4-fluorophenyl)-5-methylpyrrolo[2,3-b]pyrazine-7-carboxylic acid (30.0 mg, 0.111 mmol, 1.00 equiv) and DIEA (42.9 mg, 0.333 mmol, 3.00 equiv). The resulting mixture was stirred for 2 h at room temperature under N₂ atmosphere. The residue was purified by reverse flash chromatography with the following conditions: column, C18 silica gel; mobile phase, ACN in water, 5% to 100% gradient in 20 min; detector, UV 254 nm. This provided (6-(4-fluorophenyl)-5-methyl-5H-pyrrolo[2,3-b]pyrazin-7-yl)(3-(((2-(trifluoromethyl)pyridin-3-yl)oxy)methyl)piperidin-1-yl)methanone (27 mg 47.5%) as a white solid. ¹H NMR (400 MHz, DMSO-d₆) δ 8.54-8.13 (m, 3H), 7.90-7.47 (m, 4H), 7.41 (t, J=8.7 Hz, 2H), 4.45-4.18 (m, 1H), 4.11-4.00 (m, 1H), 3.73 (s, 4H), 3.53 (d, 1H), 3.00-2.75 (m, 2H), 1.99-1.07 (m, 5H). MS m/z: 514.5 [M+H]⁺.

### 1-(2,2-Difluoroethyl)-6-(3-(((3-(difluoromethyl)pyrazin-2-yl)oxy)methyl)piperidin-1-yl)-1H-pyrazolo[3,4-b]pyrazine (84)

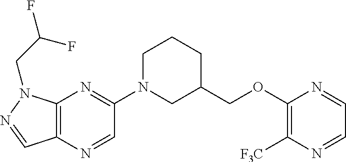 [SMILES: FC(F)Cn1ncc2ncc(N3CCCC(COc4nccnc4C(F)(F)F)C3)nc21]

Step 1: tert-butyl 3-(((3-(trifluoromethyl)pyrazin-2-yl)oxy)methyl)piperidine-1-carboxylate: Followed to General Procedure using tert-butyl 3-(hydroxymethyl)piperidine-1-carboxylate (120 mg, 0.557 mmol, 1 equiv) and 3-(trifluoromethyl)pyrazin-2(1H)-one (91.5 mg, 0.557 mmol, 1 equiv to afford tert-butyl 3-(((3-(trifluoromethyl)pyrazin-2-yl)oxy)methyl)piperidine-1-carboxylate (120 mg, 59.58%) as a white solid. MS m/z: 362 [M+H]⁺.

Step 2: 2-(piperidin-3-ylmethoxy)-3-(trifluoromethyl)pyrazine hydrochloride: Followed General Procedure B using tert-butyl 3-(((3-(trifluoromethyl)pyrazin-2-yl)oxy)methyl)piperidine-1-carboxylate (120 mg, 0.332 mmol, 1 equiv) to afford the crude product 2-(piperidin-3-ylmethoxy)-3-(trifluoromethyl)pyrazine hydrochloride (110 mg). MS m/z: 262 [M+H]⁺.

Step 3: 1-(2,2-difluoroethyl)-6-(3-(((3-(difluoromethyl)pyrazin-2-yl)oxy)methyl)piperidin-1-yl)-1H-pyrazolo[3,4-b]pyrazine: Followed General Procedure C using A 2-(piperidin-3-ylmethoxy)-3-(trifluoromethyl)pyrazine hydrochloride (50 mg, 0.191 mmol, 1 equiv) and 6-chloro-1-(2,2-difluoroethyl)pyrazolo[3,4-b]pyrazine (46.0 mg, 0.210 mmol, 1.1 equiv). This provided 1-(2,2-difluoroethyl)-6-(3-(((3-(difluoromethyl)pyrazin-2-yl)oxy)methyl)piperidin-1-yl)-1H-pyrazolo[3,4-b]pyrazine (40 mg, 47.14%) as a white solid. ¹H NMR (400 MHZ, DMSO-d₆) δ 8.58 (d, J=2.6 Hz, 1H), 8.43 (s, 1H), 8.40 (d, J=2.6 Hz, 1H), 8.12 (s, 1H), 6.40 (tt, J=55.0, 4.0 Hz, 1H), 4.71-4.59 (m, 3H), 4.49 (dd, J=10.7, 5.2 Hz, 1H), 4.41-4.31 (m, 2H), 3.24-3.12 (m, 1H), 3.02 (dd, J=13.2, 10.2 Hz, 1H), 2.20-2.11 (m, 1H), 1.95-1.86 (m, 1H), 1.85-1.76 (m, 1H), 1.63-1.40 (m, 2H). MS m/z: 444.0 [M+H]⁺.

### 1-(4-(5-Methyl-3-(3-(phenoxymethyl)piperidin-1-yl)-5H-pyrrolo[2,3-b]pyrazin-6-yl)piperidin-1-yl)ethan-1-one (85)

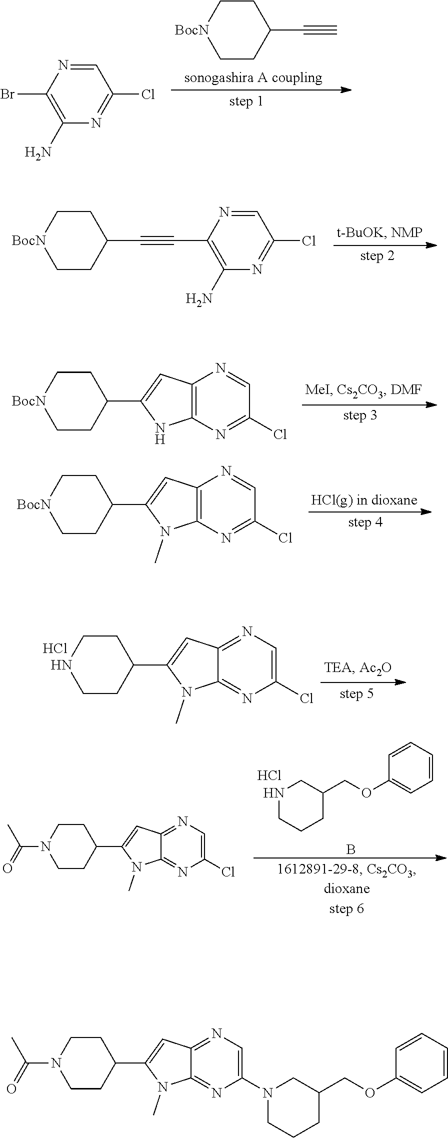

Step 1: tert-butyl 4-((3-amino-5-chloropyrazin-2-yl)ethynyl)piperidine-1-carboxylate: A solution of 3-bromo-6-chloropyrazin-2-amine (500 mg, 2.40 mmol, 1 equiv) and tert-butyl 4-ethynylpiperidine-1-carboxylate (602 mg, 2.88 mmol, 1.2 equiv) and CuI (45.7 mg, 0.24 mmol, 0.1 equiv) and Pd(PPh₃)₂Cl₂ (168 mg, 0.24 mmol, 0.1 equiv) and TEA (728 mg, 7.19 mmol, 3 equiv) in THF (8 mL) was stirred for 2 h at 80° C. The resulting mixture was concentrated under reduced pressure. The residue was purified by silica gel column chromatography, eluted with PE/EA (1:1) to afford tert-butyl 4-((3-amino-5-chloropyrazin-2-yl)ethynyl)piperidine-1-carboxylate (700 mg, 86.6%) as a yellow solid. MS m/z: 337 [M+H]⁺.

Step 2: tert-butyl 4-(3-chloro-5H-pyrrolo[2,3-b]pyrazin-6-yl)piperidine-1-carboxylate: A solution of tert-butyl 4-((3-amino-5-chloropyrazin-2-yl)ethynyl)piperidine-1-carboxylate (700 mg, 2.17 mmol, 1.00 equiv) and t-BuOK (487 mg, 4.34 mmol, 2.0 equiv) in NMP (7.0 mL) was stirred for 2 h at 80° C. The resulting mixture was diluted with EtOAc (30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford tert-butyl 4-(3-chloro-5H-pyrrolo[2,3-b]pyrazin-6-yl)piperidine-1-carboxylate (600 mg, 82.1%) as a yellow solid. MS m/z: 337 [M+H]⁺.

Step 3: tert-butyl 4-(3-chloro-5-methyl-5H-pyrrolo[2,3-b]pyrazin-6-yl)piperidine-1-carboxylate: A solution of tert-butyl 4-(3-chloro-5H-pyrrolo[2,3-b]pyrazin-6-yl)piperidine-1-carboxylate (600 mg, 1.78 mmol, 1.00 equiv) and methyl iodide (303 mg, 2.137 mmol, 1.2 equiv) and Cs₂CO₃ (1.74 g. 5.34 mmol, 3.0 equiv) in DMF (6 mL) was stirred for 2 h at room temperature. The resulting mixture was diluted with EtOAc (30 mL). The organic layer were washed with brine (20 mL), dried over anhydrous Na₂SO₄. After filtration, the filtrate was concentrated under reduced pressure. The crude product was used in the next step directly without further purification. MS m/z: 351 [M+H]⁺.

Step 4: 3-chloro-5-methyl-6-(piperidin-4-yl)-5H-pyrrolo[2,3-b]pyrazine hydrochloride: A solution of tert-butyl 4-(3-chloro-5-methyl-5H-pyrrolo[2,3-b]pyrazin-6-yl)piperidine-1-carboxylate (600 mg) in DCM (5 mL). To the above mixture was added HCl(gas) in 1,4-dioxane (5 mL) dropwise over 0.5 min at 0° C. The resulting mixture was stirred for additional 2 h at room temperature. The resulting mixture was concentrated under reduced pressure. The crude product was used in the next step directly without further purification. MS m/z: 251 [M+H]⁺.

Step 5: 1-(4-(3-chloro-5-methyl-5H-pyrrolo[2,3-b]pyrazin-6-yl)piperidin-1-yl)ethan-1-one: To a stirred solution of 3-chloro-5-methyl-6-(piperidin-4-yl)-5H-pyrrolo[2,3-b]pyrazine hydrochloride (450 mg, 1.98 mmol, 1.00 equiv) in DCM (5.0 mL) was added TEA (600 mg, 5.93 mmol, 3.0 equiv) dropwise at 0° C. To the above mixture was added acetic anhydride (242 mg, 2.37 mmol, 1.2 equiv) dropwise over 0.5 min at 0° C. The resulting mixture was stirred for additional 2 h at room temperature. The resulting mixture was diluted with EtOAc (50 mL). The combined organic layers were washed with brine (50 mL), dried over anhydrous Na₂SO₄. After filtration, the filtrate was concentrated under reduced pressure. The crude product was used in the next step directly without further purification. MS m/z: 293 [M+H]⁺.

Step 6: 1-(4-(5-methyl-3-(3-(phenoxymethyl)piperidin-1-yl)-5H-pyrrolo[2,3-b]pyrazin-6-yl)piperidin-1-yl)ethan-1-one: A solution of 1-(4-(3-chloro-5-methyl-5H-pyrrolo[2,3-b]pyrazin-6-yl)piperidin-1-yl)ethan-1-one (60 mg, 0.205 mmol, 1.00 equiv) and 3-(phenoxymethyl)piperidine hydrochloride (51.3 mg, 0.226 mmol, 1.1 equiv) and Cs₂CO₃ (133 mg, 0.410 mmol, 2 equiv) and Pd-PEPPSI-IPentCl 2-methylpyridine (o-picoline (17.24 mg, 0.021 mmol, 0.1 equiv) in dioxane (1.5 mL) was stirred for 2 h at 90° C. under nitrogen atmosphere. The resulting mixture was concentrated under reduced pressure. The residue was purified by reverse flash chromatography with the following conditions: column, C18 silica gel; mobile phase, MeCN in water, 10% to 60% gradient in 10 min; detector, UV 254 nm. This provided 1-(4-(5-methyl-3-(3-(phenoxymethyl)piperidin-1-yl)-5H-pyrrolo[2,3-b]pyrazin-6-yl)piperidin-1-yl)ethan-1-one (54.2 mg, 58.9%) as off-white solid. ¹H NMR (300 MHz, DMSO-d₆) δ 8.09 (s, 1H), 7.35-7.25 (m, 2H), 7.02-6.89 (m, 3H), 6.20 (s, 1H), 4.52 (d, J=12.9 Hz, 1H), 4.35 (d, J=13.8 Hz, 1H), 4.18 (d, J=13.2 Hz, 1H), 4.00-3.85 (d, J=6.3 Hz, 3H), 3.66 (s, 3H), 3.21 (t, J=12.3 Hz, 1H), 3.12-2.98 (m, 2H), 2.93-2.82 (m, 1H), 2.68 (t, J=13.0 Hz, 1H), 2.15-1.90 (m, 7H), 1.83-1.70 (m, 1H), 1.64-1.50 (m, 2H), 1.50-1.30 (m, 2H). MS m/z: 448.1 [M+H]⁺.

### 1-(4-(5-Methyl-3-(3-((o-tolyloxy)methyl)piperidin-1-yl)-5H-pyrrolo[2,3-b]pyrazin-6-yl)piperidin-1-yl)ethan-1-one (86)

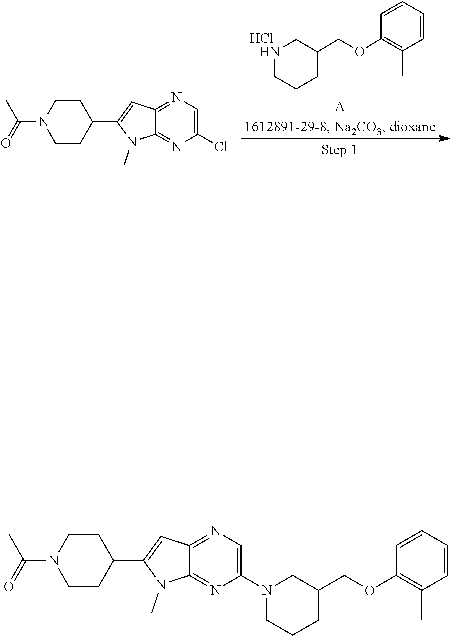 [SMILES: Cl ; Cc1ccccc1OCC1CCCNC1 ; CC(=O)N1CCC(c2cc3ncc(Cl)nc3n2C)CC1 ; "A1612891-29-8, Na2CO3, dioxane" ; CC(=O)N1CCC(c2cc3ncc(N4CCCC(COc5ccccc5C)C4)nc3n2C)CC1]

To a stirred solution of 3-((o-tolyloxy)methyl)piperidine hydrochloride (59.5 mg, 0.246 mmol, 1.20 equiv) and 1-(4-(3-chloro-5-methyl-5H-pyrrolo[2,3-b]pyrazin-6-yl)piperidin-1-yl)ethan-1-one (60.0 mg, 0.205 mmol, 1.00 equiv) in dioxane (1.00 mL) were added Na₂CO₃ (54.3 mg, 0.512 mmol, 2.50 equiv) and 1612891-29-8 (34.5 mg, 0.041 mmol, 0.200 equiv). The resulting mixture was stirred for 2 h at 90° C. under nitrogen atmosphere. The residue was purified by reverse flash chromatography with the following conditions (column, C18 gel; mobile phase, B phase: MeCN, A phase: water; 0% to 100% B gradient in 20 min; detector: UV 254/220 nm). This provided 1-(4-(5-methyl-3-(3-((o-tolyloxy)methyl)piperidin-1-yl)-5H-pyrrolo[2,3-b]pyrazin-6-yl)piperidin-1-yl)ethan-1-one (60.0 mg, 63.4%) as a white solid. ¹H NMR (300 MHz, DMSO-d₆) δ 8.09 (s, 1H), 7.17-7.12 (m, 2H), 6.95-6.92 (m, 1H), 6.89-6.77 (m, 1H), 6.20 (s, 1H), 4.55-4.46 (m, 2H), 4.22-4.18 (m, 1H), 4.03-3.85 (m, 3H), 3.66 (s, 3H), 3.25-3.16 (m, 1H), 3.10-2.95 (m, 2H), 2.90-2.83 (m, 1H), 2.72-2.63 (m, 1H), 2.24 (s, 3H), 2.04 (s, 4H), 1.94 (s, 3H), 1.80-1.76 (m, 1H), 1. 60-1.56 (m, 2H), 1.50-1.34 (m, 2H). MS m/z: 462.3 [M+H]⁺.

### 1-(4-(3-(3-(Fluoro(o-tolyloxy)methyl)piperidin-1-yl)-5-methyl-5H-pyrrolo[2,3-b]pyrazin-6-yl)piperidin-1-yl)ethan-1-one (87)

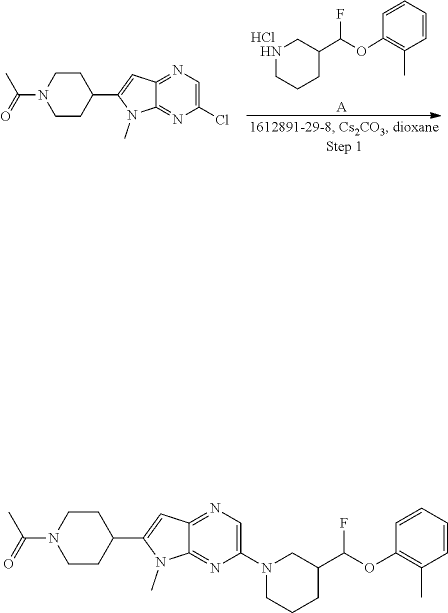

To a stirred solution of 1-(4-(3-chloro-5-methyl-5H-pyrrolo[2,3-b]pyrazin-6-yl)piperidin-1-yl)ethan-1-one (120 mg, 0.410 mmol, 1.00 equiv) and (3-(fluoro(o-tolyloxy)methyl)piperidine hydrochloride (117 mg, 0.451 mmol, 1.100 equiv) in dioxane (3.00 mL) was added Cs₂CO₃ (267 mg, 0.820 mmol, 2.00 equiv) and 1612891-29-8 (34.5 mg, 0.041 mmol, 0.100 equiv). The resulting mixture was stirred for 2 hours at 90° C. The reaction mixture was purified by reverse flash chromatography with the following conditions: column, C18 silica gel; mobile phase, MeCN in water, 0% to 100% gradient in 20 min; detector, UV 254 nm. This provided 1-(4-(3-(3-(fluoro(o-tolyloxy)methyl)piperidin-1-yl)-5-methyl-5H-pyrrolo[2,3-b]pyrazin-6-yl)piperidin-1-yl)ethan-1-one (40.0 mg, 19.8%) as a yellow solid. ¹H NMR (400 MHZ, DMSO-d₆) δ 8.11 (s, 1H), 7.15 (t, J=8.4 Hz, 2H), 6.95 (d, J=7.9 Hz, 1H), 6.86 (t, J=7.3 Hz, 1H), 6.20 (d, J=0.7 Hz, 1H), 4.52 (d, J=12.9 Hz, 1H), 4.48-4.39 (m, 1H), 4.19 (s, 1H), 4.14 (s, 1H), 4.08 (d, J=12.8 Hz, 1H), 3.93 (d, J=13.6 Hz, 1H), 3.67 (s, 3H), 3.56-3.42 (m, 1H), 3.19 (d, J=12.8 Hz, 2H), 3.06 (t, J=11.7 Hz, 1H), 2.64 (s, 1H), 2.25 (s, 3H), 2.04 (s, 3H), 2.03-1.89 (m, 5H), 1.72 (s, 1H), 1.64-1.52 (m, 1H), 1.47-1.34 (m, 1H). MS m/z: 480.1 [M+H]⁺.

### 6-(3-((3,5-Difluorophenoxy)methyl)piperidin-1-yl)-1-(oxetan-3-yl)-1H-pyrazolo[3,4-b]pyrazine (88)

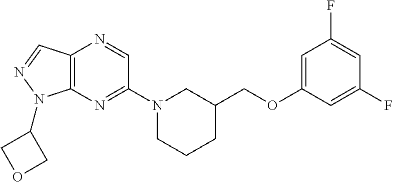 [SMILES: Fc1cc(F)cc(OCC2CCCN(c3cnc4cnn(C5COC5)c4n3)C2)c1]

Followed General Procedure C using 3-((3,5-difluorophenoxy)methyl)piperidine hydrochloride (102 mg, 0.362 mmol, 1.2 equiv) and 6-chloro-1-(oxetan-3-yl)-1H-pyrazolo[3,4-b]pyrazine (60.0 mg, 0.302 mmol, 1.00 equiv). The reaction mixture was purified by reverse flash chromatography with the following conditions: column, C18 silica gel; mobile phase, MeCN in water, 10% to 60% gradient in 15 min; detector, UV 254 nm. This provided 6-(3-((3,5-difluorophenoxy)methyl)piperidin-1-yl)-1-(oxetan-3-yl)-1H-pyrazolo[3,4-b]pyrazine (34.0 mg, 27.6%) as a yellow solid. ¹H NMR (300 MHZ, DMSO-d₆) δ 8.51 (s, 1H), 8.23 (s, 1H), 6.87-6.81 (m, 3H), 5.94-5.89 (m, 1H), 5.15-4.99 (m, 4H), 4.57-4.35 (m, 2H), 4.07-4.03 (m, 2H), 3.32-3.13 (m, 2H), 2.15-1.94 (m, 3H), 1.85-1.54 (m, 2H). MS m/z: 402.0 [M+H]⁺.

### (1R,5S,6S)-3-[1-(2,2-difluoroethyl)-1H-pyrazolo[3,4-b]pyrazin-6-yl]-6-({[3-(trifluoromethyl)pyridin-2-yl]oxy}methyl)-3-azabicyclo[3.1.0]hexane (89)

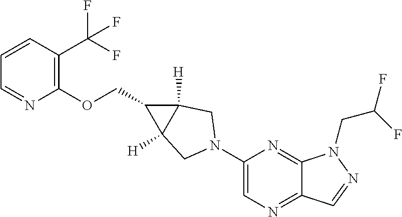 [SMILES: FC(F)Cn1ncc2ncc(N3C[C@@H]4[C@@H](COc5ncccc5C(F)(F)F)[C@@H]4C3)nc21]

Step 1: tert-butyl (1R,5S,6S)-6-({[3-(trifluoromethyl)pyridin-2-yl]oxy}methyl)-3-azabicyclo[3.1.0]hexane-3-carboxylate: Followed General Procedure D using tert-butyl (1R,5S,6S)-6-(hydroxymethyl)-3-azabicyclo[3.1.0]hexane-3-carboxylate (100 mg, 469 μmol) and 2-bromo-3-(trifluoromethyl)pyridine (106 mg, 469 μmol) to afford tert-butyl (1R,5S,6S)-6-({[3-(trifluoromethyl)pyridin-2-yl]oxy}methyl)-3-azabicyclo[3.1.0]hexane-3-carboxylate (136 mg, 81%) as a colorless oil. MS m/z: 359 [M+H]⁺.

Step 2: (1R,5S,6S)-6-({[3-(trifluoromethyl)pyridin-2-yl]oxy}methyl)-3-azabicyclo[3.1.0]hexane hydrochloride: Followed General Procedure B using tert-butyl (1R,5S,6S)-6-({[3-(trifluoromethyl)pyridin-2-yl]oxy}methyl)-3-azabicyclo[3.1.0]hexane-3-carboxylate (150 mg, 0.42 mmol) to afford (1R,5S,6S)-6-({[3-(trifluoromethyl)pyridin-2-yl]oxy}methyl)-3-azabicyclo[3.1.0]hexane hydrochloride (110 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[3-(trifluoromethyl)pyridin-2-yl]oxy}methyl)-3-azabicyclo[3.1.0]hexane: Followed General Procedure C using (1R,5S,6S)-6-({[3-(trifluoromethyl)pyridin-2-yl]oxy}methyl)-3-azabicyclo[3.1.0]hexane hydrochloride (99 mg, 0.38 mmol) and 6-chloro-1-(2,2-difluoroethyl)-1H-pyrazolo[3,4-b]pyrazine (84 mg, 0.38 mmol) to afford (1R,5S,6S)-3-[1-(2,2-difluoroethyl)-1H-pyrazolo[3,4-b]pyrazin-6-yl]-6-({[3-(trifluoromethyl)pyridin-2-yl]oxy}methyl)-3-azabicyclo[3.1.0]hexane (86 mg, 50%) as an off-white wax. ¹H NMR (500 MHz, CDCl₃) δ 8.29 (ddd, J=5.0, 1.9, 0.8 Hz, 1H), 8.02 (s, 1H), 7.91 (s, 1H), 7.87 (ddd, J=7.6, 1.9, 0.9 Hz, 1H), 6.97 (ddd, J=7.5, 5.0, 0.8 Hz, 1H), 6.22 (tt, J=55.7, 4.5 Hz, 1H), 4.65 (td, J=13.3, 4.5 Hz, 2H), 4.42 (d, J=6.7 Hz, 2H), 3.93 (d, J=10.7 Hz, 2H), 3.65 (dt, J=10.7. 2.1 Hz, 2H), 1.92-1.85 (m, 2H), 1.33-1.18 (m, 1H). MS m/z: 441.4 [M+H]⁺.

### (1R,5S,6S)-3-[1-(2,2-difluoroethyl)-1H-pyrazolo[3,4-b]pyrazin-6-yl]-6-({[4-(trifluoromethyl)pyridin-2-yl]oxy}methyl)-3-azabicyclo[3.1.0]hexane (90)

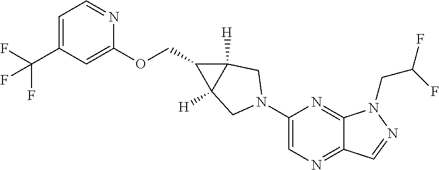 [SMILES: FC(F)Cn1ncc2ncc(N3C[C@@H]4[C@@H](COc5cc(C(F)(F)F)ccn5)[C@@H]4C3)nc21]

Step 1: tert-butyl (1R,5S,6S)-6-({[4-(trifluoromethyl)pyridin-2-yl]oxy}methyl)-3-azabicyclo[3.1.0]hexane-3-carboxylate: Followed General Procedure D using tert-butyl (1R,5S,6S)-6-(hydroxymethyl)-3-azabicyclo[3.1.0]hexane-3-carboxylate (100 mg, 469 μmol) and 2-bromo-4-(trifluoromethyl)pyridine (106 mg, 469 μmol) to afford tert-butyl (1R,5S,6S)-6-({[4-(trifluoromethyl)pyridin-2-yl]oxy}methyl)-3-azabicyclo[3.1.0]hexane-3-carboxylate (155 mg, 92%) as a colorless oil. MS m/z: 359 [M+H]⁺.

Step 2: (1R,5S,6S)-6-({[4-(trifluoromethyl)pyridin-2-yl]oxy}methyl)-3-azabicyclo[3.1.0]hexane hydrochloride: Followed General Procedure B using tert-butyl (1R,5S,6S)-6-({[4-(trifluoromethyl)pyridin-2-yl]oxy}methyl)-3-azabicyclo[3.1.0]hexane-3-carboxylate (155 mg, 0.42 mmol) to afford (1R,5S,6S)-6-({[4-(trifluoromethyl)pyridin-2-yl]oxy}methyl)-3-azabicyclo[3.1.0]hexane hydrochloride (120 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[4-(trifluoromethyl)pyridin-2-yl]oxy}methyl)-3-azabicyclo[3.1.0]hexane: Followed General Procedure C using (1R,5S,6S)-6-({[4-(trifluoromethyl)pyridin-2-yl]oxy}methyl)-3-azabicyclo[3.1.0]hexane hydrochloride (20.2 mg, 0.069 mmol) and 6-chloro-1-(2,2-difluoroethyl)-1H-pyrazolo[3,4-b]pyrazin (15 mg, 0.069 mmol) to afford (1R,5S,6S)-3-[1-(2,2-difluoroethyl)-1H-pyrazolo[3,4-b]pyrazin-6-yl]-6-({[4-(trifluoromethyl)pyridin-2-yl]oxy}methyl)-3-azabicyclo[3.1.0]hexane (10 mg, 33%) as a white solid. ¹H NMR (500 MHz, CDCl₃) δ 8.28 (d, J=5.3 Hz, 1H), 8.03 (s, 1H), 7.92 (s, 1H), 7.10-7.05 (m, 1H), 6.99 (d, J=1.5 Hz, 1H), 6.22 (tt, J=55.7, 4.5 Hz, 1H), 4.65 (td, J=13.3, 4.5 Hz, 2H), 4.30 (d, J=7.1 Hz, 2H), 3.96 (d, J=10.7 Hz, 2H), 3.66 (dt, J=10.7, 2.0 Hz, 2H), 1.92-1.79 (m, 2H), 1.30-1.26 (m, 1H). MS m/z: 441.4 [M+H]⁺.

### (1R,5S,6S)-3-[1-(2,2-difluoroethyl)-1H-pyrazolo[3,4-b]pyrazin-6-yl]-6-({[4-(trifluoromethyl)pyridin-3-yl]oxy}methyl)-3-azabicyclo[3.1.0]hexane (91)

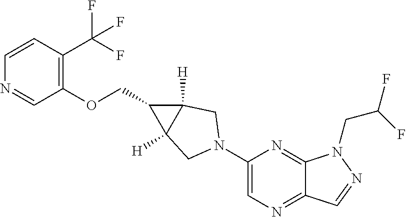 [SMILES: FC(F)Cn1ncc2ncc(N3C[C@@H]4[C@@H](COc5cnccc5C(F)(F)F)[C@@H]4C3)nc21]

Step 1: tert-butyl (1R,5S,6S)-6-({[4-(trifluoromethyl)pyridin-3-yl]oxy}methyl)-3-azabicyclo[3.1.0]hexane-3-carboxylate: Followed General Procedure A using tert-butyl (1R,5S,6S)-6-(hydroxymethyl)-3-azabicyclo[3.1.0]hexane-3-carboxylate (100 mg, 469 μmol) and 4-(trifluoromethyl)pyridin-3-ol (77 mg, 469 μmol) to afford tert-butyl (1R,5S,6S)-6-({[4-(trifluoromethyl)pyridin-3-yl]oxy}methyl)-3-azabicyclo[3.1.0]hexane-3-carboxylate (53 mg, 32%) as a colorless oil. MS m/z: 359 [M+H]⁺.

Step 2: (1R,5S,6S)-6-({[4-(trifluoromethyl)pyridin-3-yl]oxy}methyl)-3-azabicyclo[3.1.0]hexane hydrochloride: Followed General Procedure B using tert-butyl (1R,5S,6S)-6-({[4-(trifluoromethyl)pyridin-3-yl]oxy}methyl)-3-azabicyclo[3.1.0]hexane-3-carboxylate (53 mg, 0.15 mmol) to afford (1R,5S,6S)-6-({[4-(trifluoromethyl)pyridin-3-yl]oxy}methyl)-3-azabicyclo[3.1.0]hexane hydrochloride (40 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[4-(trifluoromethyl)pyridin-3-yl]oxy}methyl)-3-azabicyclo[3.1.0]hexane: Followed General Procedure C using (1R,5S,6S)-6-({[4-(trifluoromethyl)pyridin-3-yl]oxy}methyl)-3-azabicyclo[3.1.0]hexane hydrochloride (20.2 mg, 0.069 mmol) and 6-chloro-1-(2,2-difluoroethyl)-1H-pyrazolo[3,4-b]pyrazine (15 mg, 0.069 mmol) to afford (1R,5S,6S)-3-[1-(2,2-difluoroethyl)-1H-pyrazolo[3,4-b]pyrazin-6-yl]-6-({[4-(trifluoromethyl)pyridin-3-yl]oxy}methyl)-3-azabicyclo[3.1.0]hexane (22 mg, 73%) as a light-yellow oil. ¹H NMR (500 MHz, CDCl₃) δ 8.46 (s, 1H), 8.39 (d, J=4.8 Hz, 1H), 8.04 (s, 1H), 7.94 (s, 1H), 7.47 (d, J=4.9 Hz, 1H), 6.23 (tt, J=55.7, 4.5 Hz, 2H), 4.66 (td, J=13.3, 4.5 Hz, 2H), 4.22 (d, J=6.3 Hz, 2H), 3.96 (d, J=10.7 Hz, 2H), 3.68 (dt, J=10.7, 2.1 Hz, 2H), 1.93 (d, J=3.6 Hz, 2H), 1.28 (dd, J=6.4, 3.4 Hz, 1H). MS m/z: 441.4 [M+H]⁺.

### (1R,5S,6S)-3-[1-(2,2-difluoroethyl)-1H-pyrazolo[3,4-b]pyrazin-6-yl]-6-({[2-(trifluoromethyl)pyridin-4-yl]oxy}methyl)-3-azabicyclo[3.1.0]hexane (92)

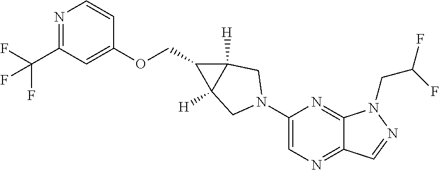 [SMILES: FC(F)Cn1ncc2ncc(N3C[C@@H]4[C@@H](COc5ccnc(C(F)(F)F)c5)[C@@H]4C3)nc21]

Step 1: tert-butyl (1R,5S,6S)-6-({[2-(trifluoromethyl)pyridin-4-yl]oxy}methyl)-3-azabicyclo[3.1.0]hexane-3-carboxylate: Followed General Procedure A using tert-butyl (1R,5S,6S)-6-(hydroxymethyl)-3-azabicyclo[3.1.0]hexane-3-carboxylate (100 mg, 469 μmol) and 2-(trifluoromethyl)pyridin-4-ol (77 mg, 469 μmol) to afford tert-butyl (1R,5S,6S)-6-({[2-(trifluoromethyl)pyridin-4-yl]oxy}methyl)-3-azabicyclo[3.1.0]hexane-3-carboxylate (56 mg, 33%) as a colorless oil. MS m/z: 359 [M+H]⁺.

Step 2: (1R,5S,6S)-6-({[2-(trifluoromethyl)pyridin-4-yl]oxy}methyl)-3-azabicyclo[3.1.0]hexane hydrochloride: Followed General Procedure B using tert-butyl (1R,5S,6S)-6-({[2-(trifluoromethyl)pyridin-4-yl]oxy}methyl)-3-azabicyclo[3.1.0]hexane-3-carboxylate (56 mg, 0.16 mmol) to afford (1R,5S,6S)-6-({[2-(trifluoromethyl)pyridin-4-yl]oxy}methyl)-3-azabicyclo[3.1.0]hexane hydrochloride (42 mg). MS m/z: 259 [M+H]⁺.

Step 3: (1R,5S,6S)-3-[1-(2,2-difluoroethyl)-1H-pyrazolo[3,4-b]pyrazin-6-yl]-6-({[2-(trifluoromethyl)pyridin-4-yl]oxy}methyl)-3-azabicyclo[3.1.0]hexane: Followed General Procedure C using (1R,5S,6S)-6-({[2-(trifluoromethyl)pyridin-4-yl]oxy}methyl)-3-azabicyclo[3.1.0]hexane hydrochloride (20.2 mg, 0.069 mmol) and 6-chloro-1-(2,2-difluoroethyl)-1H-pyrazolo[3,4-b]pyrazine (15 mg, 0.069 mmol) to afford (1R,5S,6S)-3-[1-(2,2-difluoroethyl)-1H-pyrazolo[3,4-b]pyrazin-6-yl]-6-({[2-(trifluoromethyl)pyridin-4-yl]oxy}methyl)-3-azabicyclo[3.1.0]hexane (17 mg, 73%) as a white solid. ¹H NMR (500 MHz, CDCl₃) δ 8.55 (d, J=5.7 Hz, 1H), 8.05 (s, 1H), 7.94 (s, 1H), 7.19 (d, J=2.4 Hz, 1H), 6.95 (dd, J=5.7, 2.4 Hz, 1H), 6.23 (tt, J=55.7, 4.5 Hz, 1H), 4.66 (td, J=13.3, 4.5 Hz, 2H), 4.04 (d, J=7.0 Hz, 2H), 4.00 (d, J=10.7 Hz, 2H), 3.69 (dt, J=10.8, 1.9 Hz, 2H), 1.90 (d, J=3.3 Hz, 2H), 1.29 (m, 1H). MS m/z: 441.4 [M+H]⁺.

### 1-(2,2-Difluoroethyl)-6-((3S,5R)-3-methyl-5-(((2-(trifluoromethyl)pyridin-3-yl)oxy)methyl) piperidin-1-yl)-1H-pyrazolo[3,4-b]pyrazine (93)

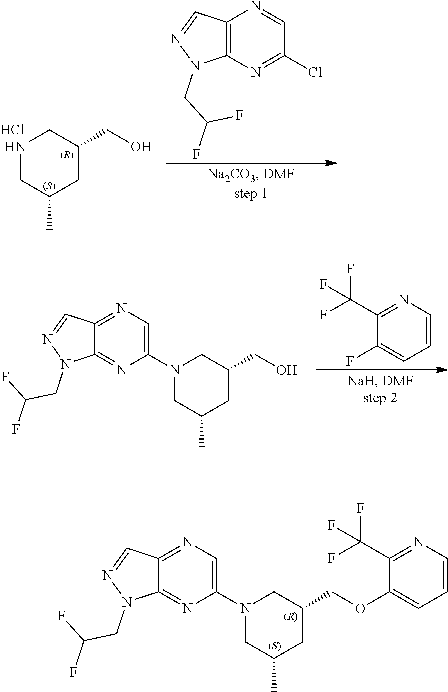

Step 1: ((3R,5S)-1-(1-(2,2-difluoroethyl)-1H-pyrazolo[3,4-b]pyrazin-6-yl)-5-methylpiperidin-3-yl)methanol: To a stirred solution of [(3R,5S)-5-methylpiperidin-3-yl]methanol hydrochloride (100 mg, 0.604 mmol, 1.00 equiv) and 6-chloro-1-(2,2-difluoroethyl) pyrazolo[3,4-b]pyrazine (132 mg, 0.604 mmol, 1 equiv) in DMF (1 mL) was added Na₂CO₃ (160 mg, 1.51 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided [(3R,5S)-1-[1-(2,2-difluoroethyl)pyrazolo[3,4-b]pyrazin-6-yl]-5-methylpiperidin-3-yl]methanol as a yellow solid (160 mg, 85.13%). MS m/z: 312 [M+H]⁺.

Step 2: 1-(2,2-difluoroethyl)-6-((3S,5R)-3-methyl-5-(((2-(trifluoromethyl)pyridine-3-yl)oxy) me-thyl)piperidin-1-yl)-1H-pyrazolo[3,4-b]pyrazine: To a stirred solution of [(3R,5S)-1-[1-(2,2-difluoroethyl)pyrazolo[3,4-b]pyrazin-6-yl]-5-methylpiperidin-3-yl]methanol (100 mg, 0.321 mmol, 1 equiv) and 3-fluoro-2-(trifluoromethyl)pyridine (79.54 mg. 0.482 mmol, 1.5 equiv) in DMF (1 mL) was added NaH (19.3 mg, 0.802 mmol, 2.5 equiv) in portions at 0° C. The resulting mixture was stirred for 2 h at room temperature. The reaction was quenched by the addition of water (2 mL) at 0° C. The residue was purified by reverse flash chromatography with the following conditions: column, C18 silica gel; mobile phase, MeCN in water, 0% to 100% gradient in 30 min; detector, UV 254 nm. This provided 3-{[(3R,5S)-1-[1-(2,2-difluoroethyl)pyrazolo[3,4-b]pyrazin-6-yl]-5-methylpiperidin-3-yl]methoxy}-2-(trifluoromethyl)pyridine as a white solid (70 mg, 47.75%). ¹H NMR (300 MHz, DMSO-d₆) δ 8.47 (s, 1H), 8.29-8.27 (m, 1H), 8.13 (s, 1H), 7.85-7.82 (m, 1H), 7.73-7.69 (m, 1H), 6.66-6.20 (m, 1H), 4.97-4.93 (m, 1H), 4.73-4.62 (m, 2H), 4.55-4.51 (m, 1H), 4.26-4.22 (m, 1H), 4.07-4.01 (m, 1H), 2.72-2.55 (m, 2H), 2.12 (s, 1H), 1.93-1.88 (m, 1H), 1.72 (s, 1H), 1.14-1.06 (m, 1H), 1.00-0.97 (m, 3H). MS m/z: 457.0 [M+H]⁺.

### 1-(2,2-Difluoroethyl)-6-((3R,5S)-3-methyl-5-(((2-(trifluoromethyl)pyridin-3-yl)oxy) methyl)piperidin-1-yl)-1H-pyrazolo[3,4-b]pyrazine (94)

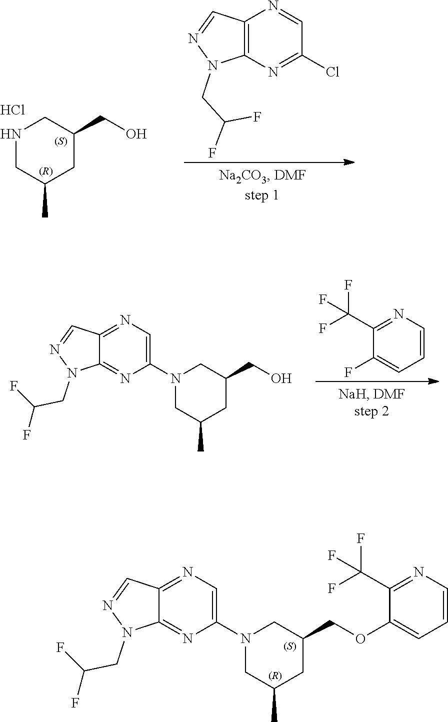

Step 1: ((3S,5R)-1-(1-(2,2-difluoroethyl)-1H-pyrazolo[3,4-b]pyrazin-6-yl)-5-methylpiperidin-3-yl)methanol: To a stirred solution of [(3S,5R)-5-methylpiperidin-3-yl]methanol hydrochloride (150 mg, 0.905 mmol, 1 equiv) and 6-chloro-1-(2,2-difluoroethyl)pyrazolo[3,4-b]pyrazine (217.72 mg, 0.996 mmol, 1.1 equiv) in DMF (2.00 mL) was added Na₂CO₃ (239.92 mg, 2.263 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided [(3S,5R)-1-[1-(2,2-difluoroethyl)pyrazolo[3,4-b]pyrazin-6-yl]-5-methylpiperidin-3-yl]methanol as a white solid (200 mg, 70.95%). MS m/z: 312[M+H]⁺.

Step 2: 1-(2,2-difluoroethyl)-6-((3R,5S)-3-methyl-5-(((2-(trifluoromethyl)pyridin-3-yl)oxy) methyl)piperidin-1-yl)-1H-pyrazolo[3,4-b]pyrazine: To a stirred solution of [(3S,5R)-1-[1-(2,2-difluoroethyl)pyrazolo[3,4-b]pyrazin-6-yl]-5-methylpiperidin-3-yl]methanol (100 mg, 0.321 mmol, 1 equiv) and 3-fluoro-2-(trifluoromethyl)pyridine (58.33 mg, 0.353 mmol, 1.1 equiv) in DMF (1.5 mL) was added NaH (11.56 mg, 0.482 mmol, 1.5 equiv) in portions at 0° C. The resulting mixture was stirred for 3 h at room temperature. The reaction was quenched by the addition of sat. NH₄Cl (aq.) (5 mL) at 0° C. The resulting mixture was extracted with EtOAc (10 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 0% to 100% gradient in 30 min; detector, UV 254 nm. This provided 3-{[(3S,5R)-1-[1-(2,2-difluoroethyl)pyrazolo[3,4-b]pyrazin-6-yl]-5-methylpiperidin-3-yl]methoxy}-2-(trifluoromethyl)pyridine as a white solid (110 mg, 74.66%). H NMR (400 MHZ, DMSO-d₆) δ 8.47 (s, 1H), 8.27 (d, J=4.5 Hz, 1H), 8.13 (s, 1H), 7.83 (d, J=8.6 Hz, 1H), 7.73-7.68 (m, 1H), 6.57-6.24 (m, 1H), 4.94 (d, J=13.0 Hz, 1H), 4.73-4.62 (m, 2H), 4.52 (d, J=12.7 Hz, 1H), 4.26-4.20 (m, 1H), 4.03 (t, J=8.9 Hz, 1H), 2.72-2.54 (m, 2H), 2.12 (d, J=4.2 Hz, 1H), 1.90 (d, J=12.8 Hz, 1H), 1.72 (s, 1H), 1.10-0.94 (m, 4H). MS m/z: 457.2 [M+H]⁺

### 1-(2,2-Difluoroethyl)-6-((3R,5R)-3-methyl-5-(((2-(trifluoromethyl)pyridin-3-yl)oxy)methyl) piperidin-1-yl)-1H-pyrazolo[3,4-b]pyrazine (95)

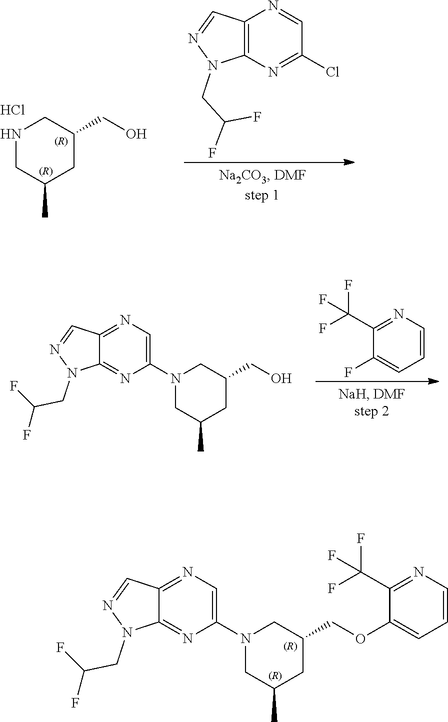

Step 1: ((3R,5R)-1-(1-(2,2-difluoroethyl)-1H-pyrazolo[3,4-b]pyrazin-6-yl)-5-methylpiperidin-3-yl)methanol: To a stirred solution of [(3R,5R)-5-methylpiperidin-3-yl]methanol hydrochloride (100 mg, 0.604 mmol, 1 equiv) and 6-chloro-1-(2,2-difluoroethyl)pyrazolo[3,4-b]pyrazine (131.95 mg, 0.604 mmol, 1 equiv) in DMF (1 mL) was added Na₂CO₃ (159.95 mg, 1.510 mmol, 2.5 equiv). The resulting mixture was stirred for 2 h at 100° C. The residue was purified by reverse flash chromatography with the following conditions: column, C18 silica gel; mobile phase, MeCN in water, 0% to 100% gradient in 30 min; detector, UV 254 nm. This provided [(3R,5R)-1-[1-(2,2-difluoroethyl)pyrazolo[3,4-b]pyrazin-6-yl]-5-methylpiperidin-3-yl]methanol as a yellow solid (160 mg, 85.13%). MS m/z: 312 [M+H]⁺.

Step 2: 1-(2,2-difluoroethyl)-6-((3R,5R)-3-methyl-5-(((2-(trifluoromethyl)pyridin-3-yl)oxy) methyl) piperidin-1-yl)-1H-pyrazolo[3,4-b]pyrazine: To a stirred solution of [(3R,5R)-1-[1-(2,2-difluoroethyl)pyrazolo[3,4-b]pyrazin-6-yl]-5-methylpiperidin-3-yl]methanol (150 mg, 0.482 mmol, 1 equiv) and NaH (17.34 mg, 0.723 mmol, 1.5 equiv) in DMF (2 mL) was added 3-fluoro-2-(trifluoromethyl)pyridine (87.49 mg, 0.530 mmol, 1.1 equiv) at room temperature. The resulting mixture was stirred for 1 h at room temperature. The reaction was monitored by LCMS. Desired product could be detected by LCMS. The residue was purified by reverse flash chromatography with the following conditions: column, silica gel; mobile phase, MeCN in water, 10% to 50% gradient in 20 min; detector, UV 254 nm to afford 3-{[(3R,5R)-1-[1-(2,2-difluoroethyl)pyrazolo[3,4-b]pyrazin-6-yl]-5-methylpiperidin-3-yl]methoxy}-2-(trifluoromethyl)pyridine as a white solid (170 mg, 75.84%). ¹H NMR (400 MHZ, DMSO-d₆) δ 8.40 (s, 1H), 8.25-8.22 (m, 1H), 8.09 (s, 1H), 7.76-7.71 (m, 1H), 7.65-7.60 (m, 1H), 6.52-6.21 (t, J=4.0 Hz, 1H), 4.64-4.54 (m, 2H), 4.16-4.10 (d, J=7.2 Hz, 2H), 3.99-3.90 (m, 2H), 3.87-3.80 (dd, J=13.2, 6.0 Hz, 1H), 3.42-3.35 (m, 1H), 2.40-2.31 (m, 1H), 2.05-1.93 (m, 1H), 1.84-1.73 (m, 1H), 1.60-1.51 (m, 1H), 0.97-0.92 (d, J=6.6 Hz, 3H). MS m/z: 457.2 [M+H]⁺.

### (2-(4-Fluorophenyl)-1-methyl-1H-imidazo[4,5-b]pyrazin-6-yl)(3-(((2-(trifluoromethyl)pyridin-3-yl)oxy)methyl)piperidin-1-yl)methanone (96)

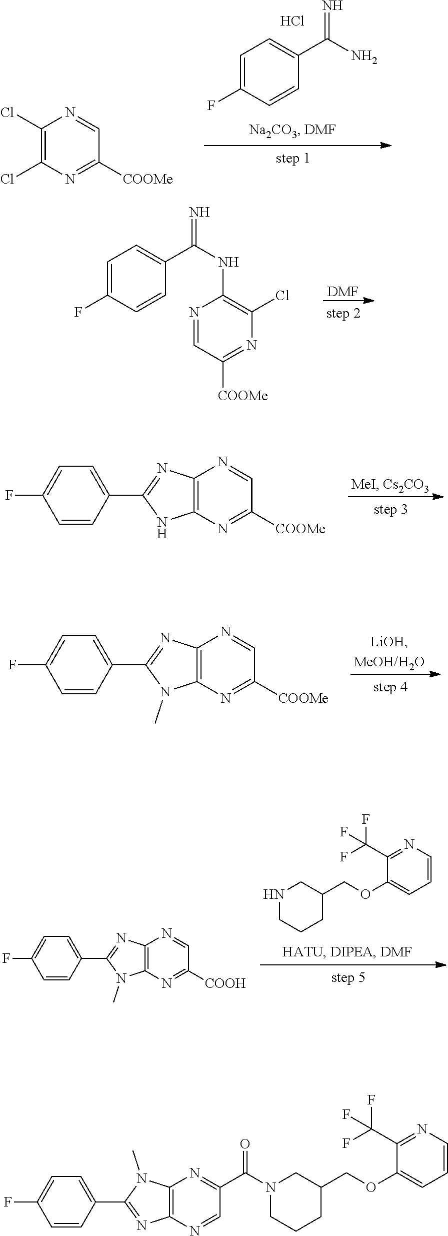

Step 1: methyl 6-chloro-5-(4-fluorobenzimidamido)pyrazine-2-carboxylate: A solution of methyl 5,6-dichloropyrazine-2-carboxylate (400 mg, 1.93 mmol, 1 equiv), 4-fluorobenzenecarboximidamide (293 mg, 2.13 mmol, 1.1 equiv) and Na₂CO₃ (409 mg, 3.86 mmol, 2 equiv) in DMF (3 mL) was stirred for 2 h at 100° C. The resulting mixture was diluted with EtOAc (30 mL). The organic layer was washed with water (3×15 mL), dried over anhydrous Na₂SO₄. After filtration, the filtrate was concentrated under reduced pressure. The residue was purified by silica gel column chromatography, eluted with PE/EA (1:1) to afford methyl 6-chloro-5-(4-fluorobenzimidamido)pyrazine-2-carboxylate as a yellow solid (500 mg, 83.8%). MS m/z: 309 [M+H]⁺.

Step 2: methyl 2-(4-fluorophenyl)-1H-imidazo[4,5-b]pyrazine-6-carboxylate: A solution of methyl 6-chloro-5-(4-fluorobenzimidamido)pyrazine-2-carboxylate (500 mg, 1.62 mmol, 1 equiv) in DMF (5 mL) was stirred for overnight at 120° C. The residue was purified by reverse flash chromatography with the following conditions: column, C18 silica gel; mobile phase, MeCN in water, 0% to 100% gradient in 30 min; detector, UV 254 nm. This provided methyl 2-(4-fluorophenyl)-1H-imidazo[4,5-b]pyrazine-6-carboxylate as a yellow solid (150 mg, 34.0%). MS m/z: 273 [M+H]⁺.

Step 3: methyl 2-(4-fluorophenyl)-1-methyl-1H-imidazo[4,5-b]pyrazine-6-carboxylate: A solution of methyl 2-(4-fluorophenyl)-1H-imidazo[4,5-b]pyrazine-6-carboxylate (150 mg, 0.551 mmol, 1 equiv), MeI (117 mg, 0.827 mmol, 1.5 equiv) and Cs₂CO₃ (359 mg, 1.10 mmol, 2 equiv) in DMF (5 mL) was stirred for overnight at room temperature. The residue was purified by reverse flash chromatography with the following conditions: column, C18 silica gel; mobile phase, MeCN in water, 10% to 100% gradient in 10 min; detector, UV 254 nm. This provided methyl 2-(4-fluorophenyl)-1-methyl-1H-imidazo[4,5-b]pyrazine-6-carboxylate as a yellow solid (80 mg, 50.7%). MS m/z: 287 [M+H]⁺.

Step 4: 2-(4-fluorophenyl)-1-methyl-1H-imidazo[4,5-b]pyrazine-6-carboxylic acid: A solution of methyl 2-(4-fluorophenyl)-1-methyl-1H-imidazo[4,5-b]pyrazine-6-carboxylate (80 mg, 0.279 mmol, 1 equiv) and LiOH (8.03 mg, 0.335 mmol, 1.2 equiv) in MeOH (1 mL)/H₂O (1 mL) was stirred for overnight at room temperature. The resulting mixture was concentrated under vacuum. The crude product was used in the next step directly without further purification. MS m/z: 273 [M+H]⁺.

Step 5: (2-(4-fluorophenyl)-1-methyl-1H-imidazo[4,5-b]pyrazin-6-yl)(3-(((2-(trifluoromethyl)pyridin-3-yl)oxy)methyl)piperidin-1-yl)methanone: A solution of 2-(4-fluorophenyl)-1-methyl-1H-imidazo[4,5-b]pyrazine-6-carboxylic acid (50 mg, 0.184 mmol, 1 equiv) and DIPEA (47.5 mg, 0.368 mmol, 2 equiv) in DMF (1.5 mL) was stirred for 2 h at room temperature. The resulting mixture was diluted with EtOAc (10 mL). The organic layer was washed with water (3×10 mL), dried over anhydrous Na₂SO₄. After filtration, the filtrate was concentrated under reduced pressure. The crude product was purified by Prep-HPLC to afford (2-(4-fluorophenyl)-1-methyl-1H-imidazo[4,5-b]pyrazin-6-yl)(3-(((2-(trifluoromethyl)pyridin-3-yl)oxy)methyl)piperidin-1-yl)methanone as a white solid (11 mg, 11.19%). ¹H NMR (400 MHZ, DMSO-d₆) δ 8.67 (d, J=41.9 Hz, 1H), 8.22 (dd, J=47.1, 4.5 Hz, 1H), 8.13-8.02 (m, 2H), 7.90-7.60 (m, 2H), 7.56-7.47 (m, 2H), 4.47 (dd, J=110.5, 12.7 Hz, 1H), 4.27-4.05 (m, 1H), 3.99-3.70 (m, 5H), 3.19-2.85 (m, 2H), 2.20-2.04 (m, 1H), 1.94-1.40 (m, 4H). MS m/z: 514.9 [M+H]⁺.

### (2-(4-Fluorophenyl)-1-methyl-1H-imidazo[4,5-b]pyrazin-5-yl)(3-(((2-(trifluoromethyl)pyridin-3-yl)oxy)methyl)piperidin-1-yl)methanone (97)

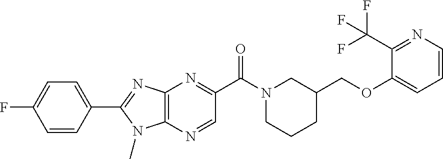 [SMILES: Cn1c(-c2ccc(F)cc2)nc2nc(C(=O)N3CCCC(COc4cccnc4C(F)(F)F)C3)cnc21]

Followed General Procedure E using 2-(4-fluorophenyl)-1-methylimidazo[4,5-b]pyrazine-5-carboxylic acid (50 mg, 0.184 mmol, 1 equiv) and 3-(piperidin-3-ylmethoxy)-2-(trifluoromethyl)pyridine (52.6 mg, 0.202 mmol, 1.1 equiv) to afford (2-(4-fluorophenyl)-1-methyl-1H-imidazo[4,5-b]pyrazin-5-yl)(3-(((2-(trifluoromethyl)pyridin-3-yl)oxy)methyl)piperidin-1-yl)methanone as a white solid (30 mg, 31.7%). ¹H NMR (400 MHz, DMSO-d₆) δ 8.56 (d, J=68.1 Hz, 1H), 8.22 (dd, J=46.9, 4.5 Hz, 1H), 8.12-8.03 (m, 2H), 7.89-7.59 (m, 2H), 7.56-7.46 (m, 2H), 4.67-4.12 (m, 2H), 4.08-3.85 (m, 4H), 3.83-3.68 (m, 1H), 3.20-2.86 (m, 2H), 2.19-1.40 (m, 5H). MS m/z: 514.9 [M+H]⁺.

### 1-(Oxetan-3-yl)-6-((1R,5S,6r)-6-(((2-(trifluoromethyl)pyridin-3-yl)oxy)methyl)-3-azabicyclo[3.1.0]hexan-3-yl)-1H-pyrazolo[3,4-b]pyrazine (98)

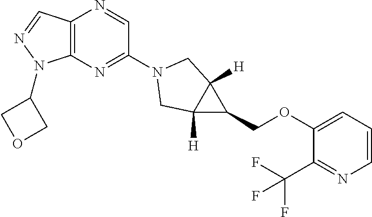 [SMILES: FC(F)(F)c1ncccc1OC[C@@H]1[C@H]2CN(c3cnc4cnn(C5COC5)c4n3)C[C@@H]12]

Followed General Procedure C using (1R,5S,6r)-6-(((2-(trifluoromethyl)pyridin-3-yl)oxy)methyl)-3-azabicyclo[3.1.0]hexane hydrochloride (67.4 mg, 0.261 mmol, 1.10 equiv) and 6-chloro-1-(oxetan-3-yl)-1H-pyrazolo[3,4-b]pyrazine (50 mg, 0.237 mmol, 1.00 equiv). The resulting mixture was stirred for 2 h at 100° C. The residue was purified by reverse flash chromatography with the following conditions (column, C18 gel; mobile phase, B phase: MeCN, A phase: water; 0% to 100% B gradient in 20 min; detector: UV 254/220 nm). This provided 1-(oxetan-3-yl)-6-((1R,5S,6r)-6-(((2-(trifluoromethyl)pyridin-3-yl)oxy)methyl)-3-azabicyclo[3.1.0]hexan-3-yl)-1H-pyrazolo[3,4-b]pyrazine as a yellow solid (59.0 mg, 54.0%). ¹H NMR (300 MHz, DMSO-d₆) δ 8.33-8.32 (m, 1H), 8.25 (s, 1H), 8.15 (s, 1H), 7.88 (d, J=8.1 Hz, 1H), 7.75-7.72 (m, 1H), 6.01-5.90 (m, 1H), 5.12 (t, J=6.4 Hz, 2H), 5.04-5.01 (m, 2H), 4.27 (d, J=6.8 Hz, 2H), 3.95 (d, J=11.0 Hz, 2H), 3.71-3.63 (m, 2H), 1.96 (d, J=3.3 Hz, 2H), 1.23-1.19 (m, 1H). MS m/z: 433.1 [M+H]⁺.

### 1-(2,2-Difluoroethyl)-6-((3S,5S)-3-methyl-5-(((2-(trifluoromethyl)pyridin-3-yl)oxy) methyl)piperidin-1-yl)-1H-pyrazolo[3,4-b]pyrazine (100)

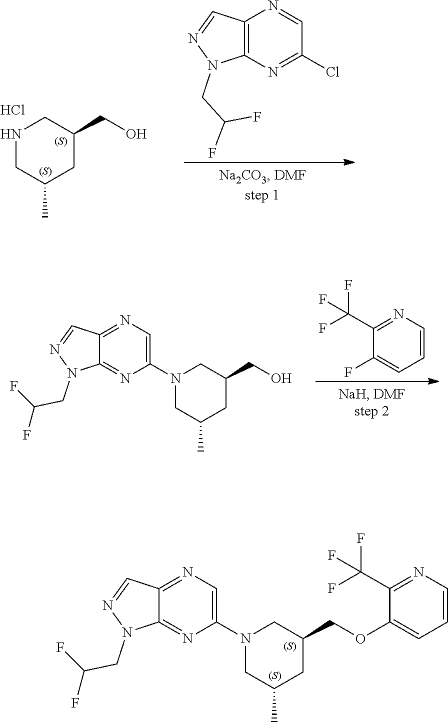

Step 1: ((3S,5S)-1-(1-(2,2-difluoroethyl)-1H-pyrazolo[3,4-b]pyrazin-6-yl)-5-methylpiperidin-3-yl)methanol: A solution of [(3S,5S)-5-methylpiperidin-3-yl]methanol (100 mg, 0.774 mmol, 1.00 equiv), Na₂CO₃ (164.06 mg, 1.548 mmol, 2 equiv) and 6-chloro-1-(2,2-difluoroethyl)pyrazolo[3,4-b]pyrazine (169.18 mg, 0.774 mmol, 1.00 equiv) in DMF (2 mL) was stirred for 2 h at 100° C. The resulting mixture was extracted with EtOAc (40 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (1:1) to afford ((3S,5S)-1-(1-(2,2-difluoroethyl)-1H-pyrazolo[3,4-b]pyrazin-6-yl)-5-methylpiperidin-3-yl)methanol as an off-white wax (150 mg, 62.25%). MS m/z: 312 [M+H]⁺.

Step 2: 1-(2,2-difluoroethyl)-6-((3S,5S)-3-methyl-5-(((2-(trifluoromethyl)pyridin-3-yl)oxy)me-thyl)piperidin-1-yl)-1H-pyrazolo[3,4-b]pyrazine: To a stirred solution of ((3S,5S)-1-(1-(2,2-difluoroethyl)-1H-pyrazolo[3,4-b]pyrazin-6-yl)-5-methylpiperidin-3-yl)methanol (150 mg, 0.482 mmol, 1 equiv) and NaH (13.87 mg, 0.578 mmol, 1.2 equiv) in DMF (3 mL) was added 3-fluoro-2-(trifluoromethyl)pyridine (87.5 mg, 0.530 mmol, 1.1 equiv) at 0° C. under argon atmosphere. The resulting mixture was diluted with EtOAc (30 mL). The combined organic layers were washed with water (3×20 mL), dried over anhydrous Na₂SO₄. After filtration, the filtrate was concentrated under reduced pressure. The crude product (150 mg) was purified by Prep-HPLC to afford 1-(2,2-difluoroethyl)-6-((3S,5S)-3-methyl-5-(((2-(trifluoromethyl)pyridin-3-yl)oxy)methyl)piperidin-1-yl)-1H-pyrazolo[3,4-b]pyrazine as a white solid (103 mg, 46.37%). ¹H NMR (300 MHz, DMSO-d₆) δ 8.41 (s, 1H), 8.24 (dd, J=4.6, 1.1 Hz, 1H), 8.09 (s, 1H), 7.77-7.72 (m, 1H), 7.63 (dd, J=8.6, 4.5 Hz, 1H), 6.40-6.35 (m, 1H), 4.69-4.54 (m, 2H), 4.14 (d, J=7.0 Hz, 2H), 4.00-3.90 (m, 2H), 3.89-3.79 (m, 1H), 3.45-3.36 (m, 1H), 2.42-2.27 (m, 1H), 2.06-1.92 (m, 1H), 1.87-1.77 (m, 1H), 1.63-1.52 (m, 1H), 0.95 (d, J=6.7 Hz, 3H). MS m/z: 457.0 [M+H]⁺.

### (1R,5S,6S)-3-[1-(2,2,2-Trifluoroethyl)-1H-pyrazolo[3,4-b]pyrazin-6-yl]-6-({[2-(trifluoromethyl)pyridin-3-yl]oxy}methyl)-3-azabicyclo[3.1.0]hexane (101)

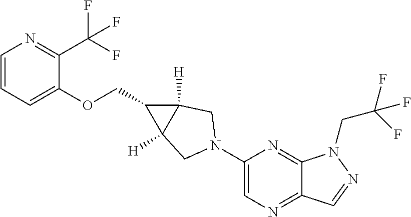 [SMILES: FC(F)(F)Cn1ncc2ncc(N3C[C@@H]4[C@@H](COc5cccnc5C(F)(F)F)[C@@H]4C3)nc21]

Step 1: tert-butyl (1R,5S,6S)-6-({[2-(trifluoromethyl)pyridin-3-yl]oxy}methyl)-3-azabicyclo[3.1.0]hexane-3-carboxylate: Followed General Procedure A using tert-butyl (1R,5S,6S)-6-(hydroxymethyl)-3-azabicyclo[3.1.0]hexane-3-carboxylate (250 mg, 1.17 mmol) and 2-(trifluoromethyl)pyridin-3-ol (191 mg, 1.17 mmol) to afford tert-butyl (1R,5S,6S)-6-({[2-(trifluoromethyl)pyridin-3-yl]oxy}methyl)-3-azabicyclo[3.1.0]hexane-3-carboxylate as a colorless oil (390 mg, 93%). MS m/z: 359 [M+H]⁺.

Step 2: (1R,5S,6S)-6-({[2-(trifluoromethyl)pyridin-3-yl]oxy}methyl)-3-azabicyclo[3.1.0]hexane hydrochloride: Followed General Procedure B using tert-butyl (1R,5S,6S)-6-({[2-(trifluoromethyl)pyridin-3-yl]oxy}methyl)-3-azabicyclo[3.1.0]hexane-3-carboxylate (390 mg, 1.09 mmol) to afford (1R,5S,6S)-6-({[2-(trifluoromethyl)pyridin-3-yl]oxy}methyl)-3-azabicyclo[3.1.0]hexane hydrochloride (300 mg). MS m/z: 259 [M+H]⁺.

Step 3: (1R,5S,6S)-3-[1-(2,2,2-trifluoroethyl)-1H-pyrazolo[3,4-b]pyrazin-6-yl]-6-({[2-(trifluoromethyl)pyridin-3-yl]oxy}methyl)-3-azabicyclo[3.1.0]hexane: Followed General Procedure C using (1R,5S,6S)-6-({[2-(trifluoromethyl)pyridin-3-yl]oxy}methyl)-3-azabicyclo[3.1.0]hexane hydrochloride (16.2 mg, 0.055 mmol) and 6-chloro-1-(2,2-difluoroethyl)-1H-pyrazolo[3,4-b]pyrazine (13 mg, 0.055 mmol) to afford (1R,5S,6S)-3-[1-(2,2,2-trifluoroethyl)-1H-pyrazolo[3,4-b]pyrazin-6-yl]-6-({[2-(trifluoromethyl)pyridin-3-yl]oxy}methyl)-3-azabicyclo[3.1.0]hexane (14 mg, 56%) as a white solid. ¹H NMR (500 MHz, DMSO-d₆) δ 8.25 (dd, J=4.5, 1.1 Hz, 1H), 8.18 (s, 1H), 8.11 (s, 1H), 7.82-7.79 (m, 1H), 7.68 (dd, J=8.6, 4.5 Hz, 1H), 5.13 (q. J=9.1 Hz, 2H), 4.20 (d, J=6.9 Hz, 2H), 3.88 (d, J=11.0 Hz, 2H), 3.61 (d, J=10.9 Hz, 2H), 1.89 (s, 2H), 1.19-1.15 (m, 1H). MS m/z: 459.4 [M+H]⁺.

### 2-(6-(3-(2-(2-(Trifluoromethyl)pyridin-3-yl)propyl)piperidin-1-yl)pyrazin-2-yl)-1,3,4-thiadiazole (102)

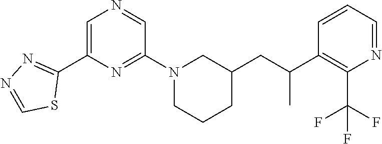 [SMILES: CC(CC1CCCN(c2cncc(-c3nncs3)n2)C1)c1cccnc1C(F)(F)F]

Followed General Procedure C using 3-[1-(piperidin-3-yl)propan-2-yl]-2-(trifluoromethyl)pyridine hydrochloride (70 mg, 0.227 mmol, 1 equiv) and 2-chloro-6-(1,3,4-thiadiazol-2-yl)pyrazine (49.5 mg, 0.250 mmol, 1.1 equiv). The residue was purified by silica gel column chromatography, eluted with EtOAc/PE (1/1) to afford the crude product. The crude product was further purified by reversed phase Combi-flash chromatography with the following conditions (column, C18 gel; mobile phase, B phase: MeCN, A phase: water; 40% to 95% B gradient in 20 min; detector: UV 254/220 nm). The pure fraction was concentrated under vacuum to afford 2-(6-(3-(2-(2-(trifluoromethyl)pyridin-3-yl)propyl)piperidin-1-yl)pyrazin-2-yl)-1,3,4-thiadiazole (24.8 mg, 25.18%) as an yellow solid. ¹H NMR (400 MHZ, DMSO-d₆) δ 9.74 (dd, J=5.5, 0.7 Hz, 1H), 8.65-8.51 (m, 2H), 8.44 (d, J=3.9 Hz, 1H), 8.17 (t, J=7.9 Hz, 1H), 7.70 (dt, J=8.8, 4.5 Hz, 1H), 4.51-4.09 (m, 2H), 3.28-3.22 (m, 1H), 3.10-2.96 (m, 1H), 2.91-2.78 (m, 0.5H), 2.71-2.61 (m, 1H), 1.86-1.75 (m, 1H), 1.73-1.54 (m, 3H), 1.54-1.41 (m, 2H), 1.31-1.13 (m, 4H). MS m/z: 435.0 [M+H]⁺.

### (1-Phenyl-1H-pyrazolo[3,4-b]pyrazin-5-yl)(3-(((2-(trifluoromethyl)pyridin-3-yl)oxy)methyl)piperidin-1-yl)methanone (103)

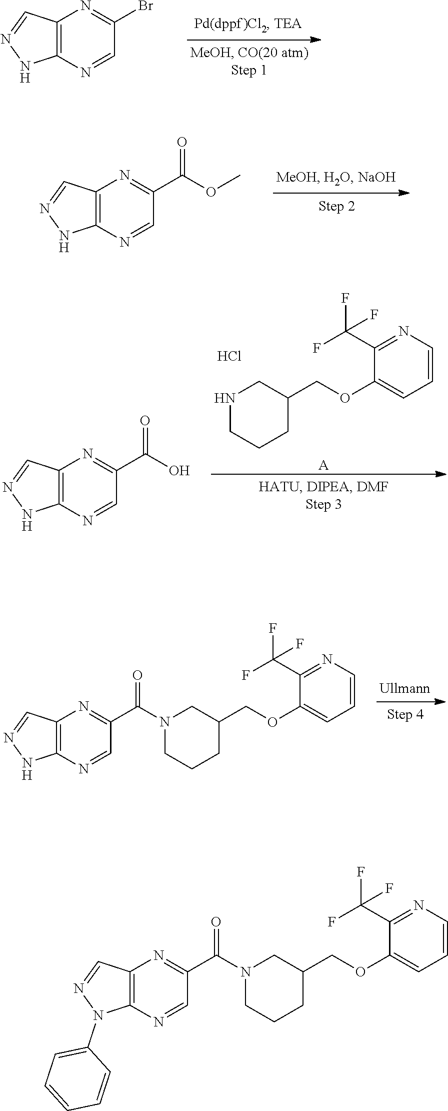

Step 1: methyl 1H-pyrazolo[3,4-b]pyrazine-5-carboxylate: A solution of 5-bromo-1H-pyrazolo[3,4-b]pyrazine (300 mg, 1.50 mmol, 1 equiv) and Pd(dppf)Cl₂ (110 mg, 0.151 mmol, 0.1 equiv) and TEA (457 mg, 4.52 mmol, 3 equiv) in MeOH (4 mL) was stirred for 4 h at 100° C. under CO atmosphere at 20 atm. The resulting mixture was concentrated under reduced pressure. The residue was purified by silica gel column chromatography, eluted with PE/EA (1:1) to afford methyl 1H-pyrazolo[3,4-b]pyrazine-5-carboxylate (180 mg, 67.0%) as a yellow oil. MS m/z: 179 [M+H]⁺.

Step 2: 1H-pyrazolo[3,4-b]pyrazine-5-carboxylic acid: A solution of methyl 1H-pyrazolo[3,4-b]pyrazine-5-carboxylate (150 mg, 0.842 mmol, 1 equiv) and NaOH (134 mg, 3.36 mmol, 4 equiv) in MeOH (1 mL) and H₂O (1 mL) was stirred for 2 h at room temperature. The mixture was acidified to pH 2 with HCl (aq.). The resulting mixture was extracted with EtOAc (3×5 mL). The combined organic layers were washed with brine (10 mL), dried over anhydrous Na₂SO₄. After filtration, the filtrate was concentrated under reduced pressure. This provided 1H-pyrazolo[3,4-b]pyrazine-5-carboxylic acid (100 mg, 72.3%) as a white solid. The crude product was used in the next step directly without further purification. MS m/z: 165 [M+H]⁺.

Step 3: (1H-pyrazolo[3,4-b]pyrazin-5-yl)(3-(((2-(trifluoromethyl)pyridin-3-yl)oxy)methyl)piperidin-1-yl)methanone: A solution of 1H-pyrazolo[3,4-b]pyrazine-5-carboxylic acid (100 mg, 0.609 mmol, 1 equiv) in DMF (2 mL) was treated with HATU (278.01 mg, 0.731 mmol, 1.2 equiv) and DIEA (236 mg, 1.82 mmol, 3 equiv) for 10 min at 0° C. under nitrogen atmosphere followed by the addition of 3-(piperidin-3-ylmethoxy)-2-(trifluoromethyl)pyridine hydrochloride (198 mg, 0.670 mmol, 1.1 equiv) at 0° C. The resulting mixture was stirred for additional 2 h at room temperature. The residue was purified by reverse flash chromatography with the following conditions: column, C18 silica gel; mobile phase MeCN in water, 10% to 100% gradient in 15 min; detector, UV 254 nm. This provided (1H-pyrazolo[3,4-b]pyrazin-5-yl)(3-(((2-(trifluoromethyl)pyridin-3-yl)oxy) methyl)piperidin-1-yl)methanone (70 mg, 28.2%) as a white solid. MS m/z: 407 [M+H]⁺.

Step 4: (1-phenyl-1H-pyrazolo[3,4-b]pyrazin-5-yl)(3-(((2-(trifluoromethyl)pyridin-3-yl)oxy)methyl)piperidin-1-yl)methanone: To a stirred solution of (1H-pyrazolo[3,4-b]pyrazin-5-yl)(3-(((2-(trifluoromethyl)pyridin-3-yl)oxy)methyl)piperidin-1-yl)methanone (50 mg, 0.123 mmol, 1 equiv) and iodophenyl (50.2 mg, 0.246 mmol, 2 equiv) in DMF (2 mL) was added Cs₂CO₃ (80.1 mg, 0.246 mmol, 2 equiv) and CuI (2.34 mg, 0.012 mmol, 0.1 equiv). The resulting mixture was stirred for 2 hours at 110° C. The reaction mixture was purified by reverse flash chromatography with the following conditions: column, C18 silica gel; mobile phase, MeCN in water, 10% to 60% gradient in 15 min; detector, UV 254 nm. This provided (1-phenyl-1H-pyrazolo[3,4-b]pyrazin-5-yl)(3-(((2-(trifluoromethyl)pyridin-3-yl)oxy)methyl)piperidin-1-yl)methanone (8 mg, 13.2%) as a light yellow solid. ¹H NMR (400 MHz, DMSO-d₆) δ 9.01-8.62 (m, 2H), 8.36-8.12 (m, 3H), 7.92-7.60 (m, 4H), 7.51-7.40 (m, 1H), 2.18-2.03 (m, 1H), 1.98-1.78 (m, 2H), 1.66-1.43 (m, 2H). MS m/z: 483.1 [M+H]⁺.

### (1-(4-fluorophenyl)-1H-pyrazolo[3,4-b]pyrazin-5-yl)(3-(((2-(trifluoromethyl)pyridin-3-yl)oxy) methyl)piperidin-1-yl)methanone (104)

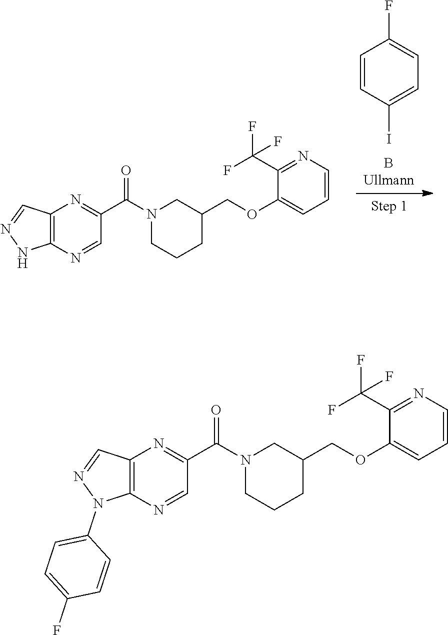 [SMILES: O=C(c1cnc2[nH]ncc2n1)N1CCCC(COc2cccnc2C(F)(F)F)C1 ; Fc1ccc(I)cc1 ; O=C(c1cnc2c(cnn2-c2ccc(F)cc2)n1)N1CCCC(COc2cccnc2C(F)(F)F)C1]

To a stirred solution of (1H-pyrazolo[3,4-b]pyrazin-5-yl)(3-(((2-(trifluoromethyl)pyridin-3-yl)oxy)methyl)piperidin-1-yl)methanone (50.0 mg, 0.123 mmol, 1.00 equiv) and 1-fluoro-4-iodobenzene (54.6 mg, 0.246 mmol, 2.00 equiv) in DMF (2 mL) was added Cs₂CO₃ (79.9 mg, 0.246 mmol, 2.00 equiv) and CuI (2.35 mg, 0.0120 mmol, 0.100 equiv). The resulting mixture was stirred for 2 hours at 110° C. The reaction mixture was purified by reverse flash chromatography with the following conditions: column, C18 silica gel; mobile phase, MeCN in water, 5% to 100% gradient in 20 min; detector, UV 254 nm. This provided (1-(4-fluorophenyl)-1H-pyrazolo[3,4-b]pyrazin-5-yl)(3-(((2-(trifluoromethyl)pyridin-3-yl)oxy)methyl)piperidin-1-yl)methanone (5.6 mg, 9.09%) as a white solid. ¹H NMR (300 MHz, DMSO-d₆) δ 9.04-8.71 (m, 2H), 8.39-8.19 (m, 3H), 7.96-7.67 (m, 2H), 7.57 (td, J=8.9, 2.1 Hz, 2H), 4.42-4.06 (m, 2H), 4.06-3.75 (m, 2H), 3.30-3.17 (m, 2H), 3.07-2.91 (m, 1H), 2.40-1.85 (m, 3H), 1.77-1.47 (m, 2H). MS m/z: 501.2 [M+H]⁺.

### (1-methyl-2-phenyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (105)

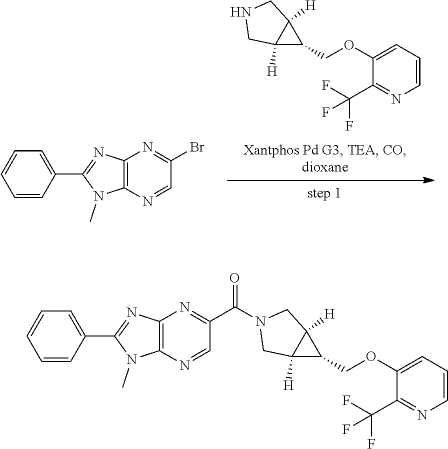 [SMILES: FC(F)(F)c1ncccc1OC[C@H]1[C@@H]2CNC[C@@H]21 ; Cn1c(-c2ccccc2)nc2nc(Br)cnc21 ; Cn1c(-c2ccccc2)nc2nc(C(=O)N3C[C@@H]4[C@@H](COc5cccnc5C(F)(F)F)[C@@H]4C3)cnc21]

A solution of 6-bromo-1-methyl-2-phenylimidazo[4,5-b]pyrazine (50 mg, 0.173 mmol, 1 equiv) TEA (35.0 mg, 0.35 mmol, 2 equiv), Xantphos Pd G3 (16.6 mg, 0.02 mmol, 0.1 equiv) and (1R,5S,6S)-6-({[2-(trifluoromethyl)pyridin-3-yl]oxy}methyl)-3-azabicyclo[3.1.0]hexane (44.6 mg, 0.173 mmol, 1 equiv) in dioxane (1 mL) was stirred for overnight at 50° C. under CO atmosphere. The resulting mixture was concentrated under vacuum. The residue was purified by silica gel column chromatography, eluted with EA to afford impure product. This was purified by reverse flash chromatography with the following conditions: column, C18 silica gel; mobile phase, MeCN in water, 10% to 100% gradient in 30 min; detector, UV 254 nm. This provided (1-methyl-2-phenyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (60 mg, 70.2%) as a white solid. ¹H NMR (300 MHZ, DMSO-d₆) δ 8.78 (s, 1H), 8.25 (dd, J=4.5, 1.2 Hz, 1H), 8.07-7.99 (m, 2H), 7.80 (d, J=8.6 Hz, 1H), 7.72-7.63 (m, 4H), 4.25-4.10 (m, 2H), 4.03-3.93 (m, 6H), 3.61 (dd, J=12.2, 3.7 Hz, 1H), 1.84-1.71 (m, 2H), 1.19-1.09 (m, 1H). MS m/z: 495 [M+H]⁺.

### (1-Methyl-2-phenyl-1H-imidazo[4,5-b]pyrazin-6-yl)((1R,5S,6r)-6-(((2-(trifluoromethyl)pyridin-3-yl)oxy)methyl)-3-azabicyclo[3.1.0]hexan-3-yl)methanone (106)

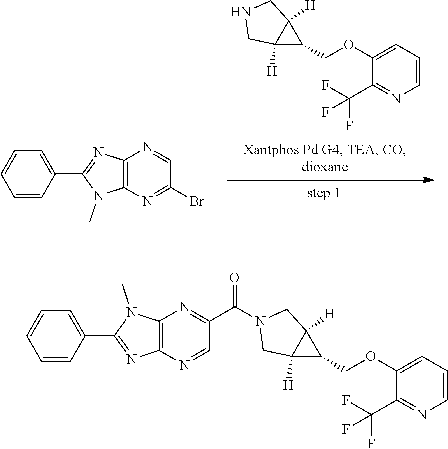 [SMILES: FC(F)(F)c1ncccc1OC[C@H]1[C@@H]2CNC[C@@H]21 ; Cn1c(-c2ccccc2)nc2ncc(Br)nc21 ; Cn1c(-c2ccccc2)nc2ncc(C(=O)N3C[C@@H]4[C@@H](COc5cccnc5C(F)(F)F)[C@@H]4C3)nc21]

A solution of 6-bromo-1-methyl-2-phenylimidazo[4,5-b]pyrazine (50 mg, 0.173 mmol, 1 equiv), (1R,5S,6S)-6-({[2-(trifluoromethyl)pyridin-3-yl]oxy}methyl)-3-azabicyclo[3.1.0]hexane (44.7 mg, 0.173 mmol, 1 equiv), TEA (35 mg, 0.346 mmol, 2 equiv) and Xantphos Pd G4 (16.6 mg, 0.017 mmol, 0.1 equiv) in dioxane (3 mL) was stirred for overnight at 50° C. under CO atmosphere. The resulting mixture was concentrated under vacuum. The residue was purified by silica gel column chromatography, eluted with EA to afford impure product. This was purified by reverse flash chromatography with the following conditions: column, C18 silica gel; mobile phase, MeCN in water, 10% to 100% gradient in 30 min; detector, UV 254 nm. This provided (1-methyl-2-phenyl-1H-imidazo[4,5-b]pyrazin-6-yl)((1R,5S,6r)-6-(((2-(trifluoromethyl)pyridin-3-yl)oxy)methyl)-3-azabicyclo[3.1.0]hexan-3-yl)methanone (35 mg, 39.7%) as a white solid. ¹H NMR (300 MHZ, DMSO-d₆) δ 8.86 (s, 1H), 8.25 (dd, J=4.5, 1.2 Hz, 1H), 8.09-7.99 (m, 2H), 7.80 (d, J=8.0 Hz, 1H), 7.72-7.62 (m, 4H), 4.27-4.09 (m, 2H), 4.08-3.89 (m, 6H), 3.61 (dd, J=12.3, 3.6 Hz, 1H), 1.85-1.73 (m, 2H), 1.20-1.10 (m, 1H). MS m/z: 495 [M+H]⁺.

### 4-(2-Oxo-2-((1R,5S,6r)-6-(((2-(trifluoromethyl)pyridin-3-yl)oxy)methyl)-3-azabicyclo[3.1.0]hexan-3-yl)ethyl)-2-phenylpyridazin-3(2H)-one (107)

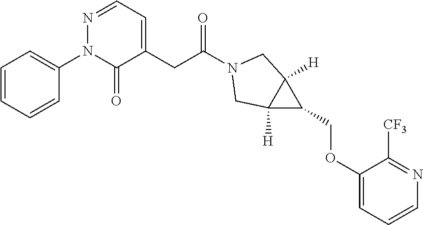 [SMILES: O=C(Cc1ccnn(-c2ccccc2)c1=O)N1C[C@@H]2[C@@H](COc3cccnc3C(F)(F)F)[C@@H]2C1]

Followed General Procedure E using 2-(3-oxo-2-phenyl-2,3-dihydropyridazin-4-yl)acetic acid (105 mg, 0.456 mmol, 1.00 equiv) and (1R,5S,6r)-6-(((2-(trifluoromethyl)pyridin-3-yl)oxy)methyl)-3-azabicyclo[3.1.0]hexane hydrochloride (147.8 mg, 0.502 mmol, 1.10 equiv). The resulting mixture was diluted with water (30 mL) and extracted with EtOAc (3×30 mL). The combined organic layers were washed with brine (30 mL), dried over anhydrous Na₂SO₄. After filtration, the filtrate was concentrated to dryness under reduced pressure. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 4-(2-oxo-2-((1R,5S,6r)-6-(((2-(trifluoromethyl)pyridin-3-yl)oxy)methyl)-3-azabicyclo[3.1.0]hexan-3-yl)ethyl)-2-phenyl pyridazin-3(2H)-one (47.0 mg, 21.6%) as a white solid. ¹H NMR (300 MHZ, DMSO-d₆) δ 8.32 (d, J=4.4 Hz, 1H), 8.08 (d, J=4.0 Hz, 1H), 7.86 (d, J=8.7 Hz, 1H), 7.76-7.72 (m, 1H), 7.60-7.56 (m, 5H), 7.43 (d, J=4.1 Hz, 1H), 4.30-4.17 (m, 2H), 3.83-3.59 (m, 5H), 3.53 (s, 0.5H), 3.40 (s, 0.5H), 1.86-1.82 (m, 1H), 1.78-1.73 (m, 1H), 1.16-1.12 (m, 1H). MS m/z: 471.2 [M+H]⁺.

### 1-(2,2-Difluoroethyl)-6-(3-(2-(2-(trifluoromethyl)pyridin-3-yl)propyl)piperidin-1-yl)-1H-pyrazolo[3,4-b]pyrazine (108)

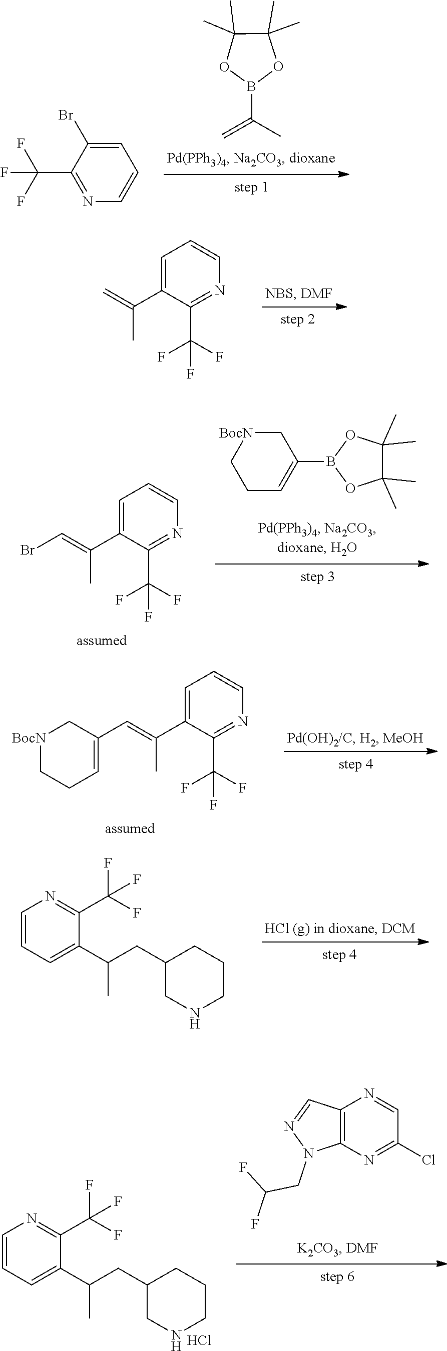

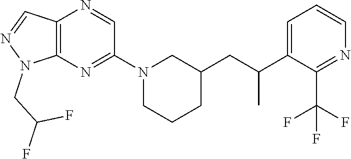 [SMILES: CC(CC1CCCN(c2cnc3cnn(CC(F)F)c3n2)C1)c1cccnc1C(F)(F)F]

Step 1: 3-(prop-1-en-2-yl)-2-(trifluoromethyl)pyridine: To the solution of 3-bromo-2-(trifluoromethyl)pyridine (900 mg, 3.98 mmol, 1 equiv) and 4,4,5,5-tetramethyl-2-(prop-1-en-2-yl)-1,3,2-dioxaborolane (1.33 g, 7.96 mmol, 2 equiv) in dioxane (10 mL)/H₂O (2 mL) were added Pd(dppf)Cl₂ (145 mg, 0.199 mmol, 0.05 equiv) and K₂CO₃ (1650 mg, 12.0 mmol, 3 equiv) under N₂ atmosphere. The result mixture was heated to 80° C. and stirred for 2 h. Desired product could be detected by LCMS. The reaction mixture was diluted by EtOAc (50 mL), washed by water (2×40 mL) and brine (1×40 mL), dried over anhydrous sodium sulfate. After filtration, the filtrate was concentrated. The residue was purified by silica gel column chromatography, eluted with EtOAc/PE=1/10 to afford 3-(prop-1-en-2-yl)-2-(trifluoromethyl)pyridine (700 mg, 93.9%) as colorless oil. MS m/z: 188 [M+H]⁺.

Step 2: 3-(1-bromoprop-1-en-2-yl)-2-(trifluoromethyl)pyridine(assumed): To a stirred solution of 3-(prop-1-en-2-yl)-2-(trifluoromethyl)pyridine (200 mg, 1.07 mmol, 1 equiv) and NBS (228 mg, 1.28 mmol, 1.2 equiv) in DMF (5 mL) was added AIBN (17.5 mg, 0.107 mmol, 0.1 equiv) at room temperature. The resulting mixture was stirred for 16 h at room temperature under nitrogen atmosphere. The reaction was monitored by LCMS. The resulting mixture was diluted with water (50 mL) and extracted with EtOAc (3×50 mL). The combined organic layers were washed with brine (1×150 mL), dried over anhydrous Na₂SO₄. After filtration, the filtrate was concentrated under reduced pressure. The residue was purified by silica gel column chromatography, eluted with PE/EA (15:1) to afford 3-(1-bromoprop-1-en-2-yl)-2-(trifluoromethyl)pyridine (assumed, 100 mg, 35.2%) as a colorless oil. MS m/z: 266 [M+H]⁺.

Step 3: tert-butyl 5-(2-(2-(trifluoromethyl)pyridin-3-yl)prop-1-en-1-yl)-3,6-dihydropyridine-1(2H)-carboxylate (assumed): To the solution of 3-(1-bromoprop-1-en-2-yl)-2-(trifluoromethyl)pyridine (380 mg, 1.43 mmol, 1 equiv) and tert-butyl 5-(4,4,5,5-tetramethyl-1,3,2-dioxaborolan-2-yl)-3,6-dihydropyridine-1(2H)-carboxylate (701 mg, 2.28 mmol, 1.6 equiv) in dioxane (10 mL)/H₂O (2 mL) were added Pd(PPh₃)+(82.5 mg, 0.071 mmol, 0.05 equiv) and Na₂CO₃ (454 mg, 4.28 mmol, 3 equiv) under N₂ atmosphere. The result mixture was heated to 60° C. and stirred overnight. Desired product could be detected by LCMS. The reaction mixture was diluted by EtOAc (50 mL), washed by water (2×40 mL) and brine (1×40 mL), dried over anhydrous sodium sulfate. After filtration, the filtrate was concentrated. The residue was purified by silica gel column chromatography, eluted with EtOAc/PE=1/2, to afford tert-butyl 5-(2-(2-(trifluoromethyl)pyridin-3-yl)prop-1-en-1-yl)-3,6-dihydropyridine-1(2H)-carboxylate (assumed, 180 mg, 34.2%) as colorless oil. MS m/z: 313 [M-tBu+H]⁺.

Step 4: tert-butyl 3-{2-[2-(trifluoromethyl)pyridin-3-yl]propyl}piperidine-1-carboxylate: To the solution of tert-butyl 5-(2-(2-(trifluoromethyl)pyridin-3-yl)prop-1-en-1-yl)-3,6-dihydropyridine-1(2H)-carboxylate (200 mg, 0.543 mmol, 1 equiv) in MeOH (3 mL) was added Pd(OH)₂/C (99.11 mg) with water. The resulted mixture was hydrogenated overnight at room temperature. Desired product could be detected by LCMS. The reaction system was filtrated through celite and the filtrate was concentrated. The crude product tert-butyl 3-{2-[2-(trifluoromethyl)pyridin-3-yl]propyl}piperidine-1-carboxylate (180 mg) was used directly for next step. MS m/z: 373 [M+H]⁺.

Step 5: 3-[1-(piperidin-3-yl)propan-2-yl]-2-(trifluoromethyl)pyridine hydrochloride: Followed General Procedure B using tert-butyl 3-{2-[2-(trifluoromethyl)pyridin-3-yl]propyl}piperidine-1-carboxylate (180 mg, 0.483 mmol, 1 equiv) to afford the crude product 3-[1-(piperidin-3-yl)propan-2-yl]-2-(trifluoromethyl)pyridine hydrochloride (160 mg) was used for next step without further purification. MS m/z: 273 [M+H]⁺.

Step 6: 1-(2,2-difluoroethyl)-6-(3-(2-(2-(trifluoromethyl)pyridin-3-yl)propyl)piperidin-1-yl)-1H-pyrazolo[3,4-b]pyrazine: Followed General Procedure C using 3-[1-(piperidin-3-yl)propan-2-yl]-2-(trifluoromethyl)pyridine hydrochloride (70 mg, 0.227 mmol, 1 equiv) and 6-chloro-1-(2,2-difluoroethyl)pyrazolo[3,4-b]pyrazine (49.56 mg, 0.227 mmol, 1 equiv). The residue was purified by silica gel column chromatography, eluted with EtOAc/PE (1/1) to afford the crude product. The crude product was further purified by reversed phase Combi-flash chromatography with the following conditions (column, C18 gel; mobile phase, B phase: MeCN, A phase: water; 40% to 95% B gradient in 20 min; detector: UV 254/220 nm). The pure fraction was concentrated under vacuum to afford 1-(2,2-difluoroethyl)-6-(3-(2-(2-(trifluoromethyl)pyridin-3-yl)propyl)piperidin-1-yl)-1H-pyrazolo[3,4-b]pyrazine (24 mg, 23.30%) as a white solid. ¹H NMR (400 MHZ, DMSO-d₆) δ 8.56 (d, J=4.4 Hz, 1H), 8.38 (d, J=3.7 Hz, 1H), 8.23-8.08 (m, 2H), 7.70 (ddd, J=7.3, 4.6, 2.2 Hz, 1H), 6.62-6.21 (m, 1H), 4.83-4.56 (m, 2H), 4.56-4.16 (m, 2H), 3.30-3.24 (m, 1H), 3.21-2.97 (m, 1H), 2.95-2.84 (m, 0.5H), 2.67-2.58 (m, 1H), 1.86-1.86 (m, 1H), 1.77-1.65 (m, 2H), 1.66-1.55 (m, 1H), 1.51-1.37 (m, 2H), 1.32-1.17 (m, 4H). MS m/z: 455.2 [M+H]⁺.

### 6-[(1R,5S,6R)-6-({[2-(Trifluoromethyl)pyridin-3-yl]oxy}methyl)-3-azabicyclo[3.1.0]hexane-3-carbonyl]-1H-indole (109)

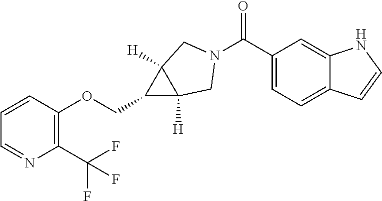 [SMILES: O=C(c1ccc2cc[nH]c2c1)N1C[C@@H]2[C@@H](COc3cccnc3C(F)(F)F)[C@@H]2C1]

Followed General Procedure E using (1R,5S,6S)-6-({[2-(trifluoromethyl)pyridin-3-yl]oxy}methyl)-3-azabicyclo[3.1.0]hexane hydrochloride (37 mg, 0.12 mmol) and 1H-indole-6-carboxylic acid (20 mg, 0.12 mmol) to afford 6-[(1R,5S,6R)-6-({[2-(trifluoromethyl)pyridin-3-yl]oxy}methyl)-3-azabicyclo[3.1.0]hexane-3-carbonyl]-1H-indole (41 mg, 82%) as a white solid. ¹H NMR (500 MHz, DMSO-d₆) δ 11.28 (t, J=2.3 Hz, 1H), 8.24 (dd, J=4.5, 1.1 Hz, 1H), 7.77 (dd, J=8.7, 1.2 Hz, 1H), 7.67 (dd, J=8.6, 4.5 Hz, 1H), 7.55 (d, J=8.2 Hz, 1H), 7.50 (dd, J=1.5, 0.8 Hz, 1H), 7.47-7.42 (m, 1H), 7.09 (dd, J=8.2, 1.4 Hz, 1H), 6.47 (td, J=2.0, 0.9 Hz, 1H), 4.13 (d, J=6.8 Hz, 2H), 4.03 (dd, J=12.8, 5.8 Hz, 1H), 3.73 (s, 1H), 3.51-3.38 (m, 2H), 1.69 (d, J=10.7 Hz, 2H), 1.02 (tt, J=6.8, 3.3 Hz, 1H). MS m/z: 402.4 [M+H]⁺.

### (1R,5S,6R)-3-[3-(Propan-2-yl)-1H-pyrazole-5-carbonyl]-6-({[2-(trifluoromethyl)pyridin-3-yl]oxy}methyl)-3-azabicyclo[3.1.0]hexane (110)

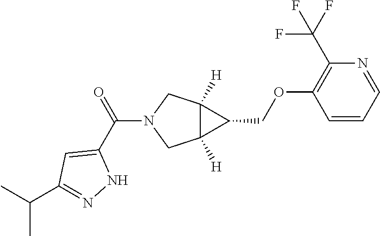 [SMILES: CC(C)c1cc(C(=O)N2C[C@@H]3[C@@H](COc4cccnc4C(F)(F)F)[C@@H]3C2)[nH]n1]

Followed General Procedure E using (1R,5S,6S)-6-({[2-(trifluoromethyl)pyridin-3-yl]oxy}methyl)-3-azabicyclo[3.1.0]hexane hydrochloride (37 mg, 0.12 mmol) and 3-(propan-2-yl)-1H-pyrazole-5-carboxylic acid (19 mg, 0.12 mmol) to afford (1R,5S,6R)-3-[3-(propan-2-yl)-1H-pyrazole-5-carbonyl]-6-({[2-(trifluoromethyl)pyridin-3-yl]oxy}methyl)-3-azabicyclo[3.1.0]hexane (32 mg, 65%) as a white solid. ¹H NMR (500 MHZ, DMSO-d₆) δ 12.92 (s, 1H), 8.24 (dd, J=4.5, 1.2 Hz, 1H), 7.78 (d, J=8.6 Hz, 1H), 7.66 (dd, J=8.6, 4.5 Hz, 1H), 6.36 (s, 1H), 4.26 (d, J=11.7 Hz, 1H), 4.22-4.06 (m, 2H), 3.86 (d, J=12.1 Hz, 1H), 3.78 (dd, J=12.0, 4.3 Hz, 1H), 3.45 (dd, J=12.2, 4.4 Hz, 1H), 2.95 (p, J=7.0 Hz, 1H), 1.80-1.64 (m, 2H), 1.21 (d, J=6.9 Hz, 6H), 0.97 (dt, J=6.9, 3.4 Hz, 1H). MS m/z: 395.2 [M+H]⁺.

### 1-(4-(6-(3-((2-(trifluoromethyl)phenoxy)methyl)piperidin-1-yl)-1H-pyrazolo[3,4-b]pyrazin-1-yl)piperidin-1-yl)ethan-1-one (111)

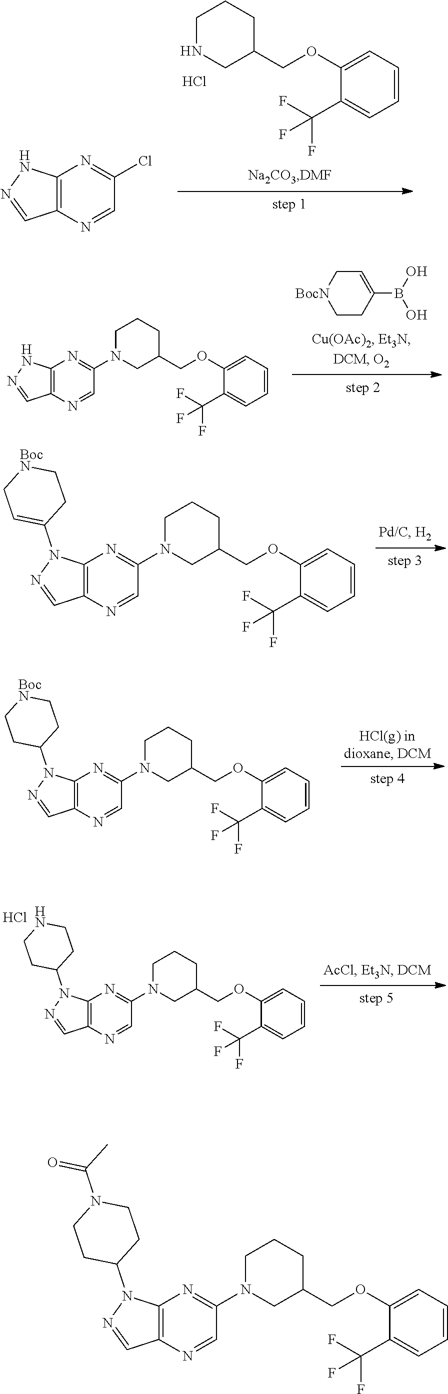

Step 1: 6-(3-((2-(trifluoromethyl)phenoxy)methyl)piperidin-1-yl)-1H-pyrazolo[3,4-b]pyrazine: A solution of 6-chloro-1H-pyrazolo[3,4-b]pyrazine (500 mg, 3.23 mmol, 1 equiv), 3-((2-(trifluoromethyl)phenoxy)methyl)piperidine (1 g, 3.88 mmol, 1.2 equiv) and Na₂CO₃ (1.03 g, 9.75 mmol, 3 equiv) in DMF (4 mL) was stirred for 3 h at 100° C. The resulting mixture was diluted with EtOAc (30 mL). The organic layer was washed with water (3×20 mL), dried over anhydrous Na₂SO₄. After filtration, the filtrate was concentrated under reduced pressure. The residue was purified by silica gel column chromatography, eluted with PE/EA to afford 6-(3-((2-(trifluoromethyl)phenoxy)methyl)piperidin-1-yl)-1H-pyrazolo[3,4-b]pyrazine (500 mg, 41.0%) as a white solid. MS m/z: 378 [M+H]⁺.

Step 2: tert-butyl 4-(6-(3-((2-(trifluoromethyl)phenoxy)methyl)piperidin-1-yl)-1H-pyrazolo[3,4-b]pyrazin-1-yl)-3,6-dihydropyridine-1(2H)-carboxylate: A solution of 6-(3-((2-(trifluoromethyl)phenoxy)methyl)piperidin-1-yl)-1H-pyrazolo[3,4-b]pyrazine (500 mg, 1.33 mmol, 1 equiv), 1-(tert-butoxycarbonyl)-3,6-dihydro-2H-pyridin-4-ylboronic acid (451 mg, 1.98 mmol, 1.5 equiv), Cu(OAc)₂ (481 mg, 2.65 mmol, 2 equiv) and Et₃N (402 mg, 3.97 mmol, 3 equiv) in DCM (10 mL) was stirred for overnight at room temperature under O₂ atmosphere. The resulting mixture was concentrated under vacuum. The residue was purified by silica gel column chromatography, eluted with PE/EA (1:1) to afford tert-butyl 4-(6-(3-((2-(trifluoromethyl)phenoxy)methyl)piperidin-1-yl)-1H-pyrazolo[3,4-b]pyrazin-1-yl)-3,6-dihydropyridine-1(2H)-carboxylate (150 mg, 20.2%) as a white solid. MS m/z: 559 [M+H]⁺.

Step 3. tert-butyl 4-(6-(3-((2-(trifluoromethyl)phenoxy)methyl)piperidin-1-yl)-1H-pyrazolo[3,4-b]pyrazin-1-yl)piperidine-1-carboxylate: A solution of tert-butyl 4-(6-(3-((2-(trifluoromethyl)phenoxy)methyl)piperidin-1-yl)-1H-pyrazolo[3,4-b]pyrazin-1-yl)-3,6-dihydropyridine-1(2H)-carboxylate (150 mg, 0.269 mmol, 1 equiv) and Pd/C (2.86 mg, 0.027 mmol, 0.1 equiv) in CH₃CH₂OH (3 mL) was stirred for overnight at room temperature under hydrogen atmosphere. The resulting mixture was filtered, and the filter cake was washed with MeOH (4×10 mL). The filtrate was concentrated under reduced pressure. The crude product (80 mg) was used in the next step directly without further purification. MS m/z: 561 [M+H]⁺.

Step 4. 1-(piperidin-4-yl)-6-(3-((2-(trifluoromethyl)phenoxy)methyl)piperidin-1-yl)-1H-pyrazolo[3,4-b]pyrazine hydrochloride: A solution of tert-butyl 4-(6-(3-((2-(trifluoromethyl)phenoxy)methyl)piperidin-1-yl)-1H-pyrazolo[3,4-b]pyrazin-1-yl)piperidine-1-carboxylate (80 mg, 0.143 mmol, 1 equiv) in HCl(gas) in 1,4-dioxane (2 mL)/DCM (2 mL) was stirred for 2 h at room temperature. The resulting mixture was concentrated under vacuum. The crude product (50 mg) was used in the next step directly without further purification. MS m/z: 461 [M+H]⁺.

Step 5. 1-(4-(6-(3-((2-(trifluoromethyl)phenoxy)methyl)piperidin-1-yl)-1H-pyrazolo[3,4-b]pyrazin-1-yl)piperidin-1-yl)ethan-1-one: A solution of 1-(piperidin-4-yl)-6-(3-((2-(trifluoromethyl)phenoxy)methyl)piperidin-1-yl)-1H-pyrazolo[3,4-b]pyrazine (30 mg, 0.065 mmol, 1 equiv), AcCl (7.67 mg, 0.098 mmol, 1.5 equiv) and Et₃N (19.8 mg, 0.195 mmol, 3 equiv) in DCM (2 mL) was stirred for 2 h at room temperature. The resulting mixture was concentrated under vacuum. The residue was purified by silica gel column chromatography, eluted with EA to afford impure compound. This was further purified by reverse flash chromatography with the following conditions: column, C18 silica gel; mobile phase, MeCN in water, 10% to 100% gradient in 30 min; detector, UV 254 nm. This provided 1-(4-(6-(3-((2-(trifluoromethyl)phenoxy)methyl)piperidin-1-yl)-1H-pyrazolo[3,4-b]pyrazin-1-yl)piperidin-1-yl)ethan-1-one (15 mg, 19.4%) as a white solid. ¹H NMR (400 MHZ, DMSO-d₆) δ 8.39 (s, 1H), 8.03 (s, 1H), 7.63 (t, J=8.8 Hz, 2H), 7.28 (d, J=8.3 Hz, 1H), 7.10 (t, J=7.6 Hz, 1H), 4.86-4.74 (m, 1H), 4.72-4.62 (m, 1H), 4.54-4.43 (m, 1H), 4.42-4.37 (m, 1H), 4.17-4.09 (m, 1H), 4.01 (t, J=8.9 Hz, 2H), 3.29-3.18 (m, 1H), 3.17-3.08 (m, 1H), 3.03-2.92 (m, 1H), 2.82-2.70 (m, 1H), 2.17-2.00 (m, 5H), 1.96-1.76 (m, 5H), 1.65-1.51 (m, 1H), 1.52-1.39 (m, 1H). MS m/z: 503.2 [M+H]⁺.

### 1-(3-methyl-4-(6-(3-((2-(trifluoromethyl)phenoxy)methyl)piperidin-1-yl)pyrazin-2-yl)piperidin-1-yl)ethan-1-one (112)

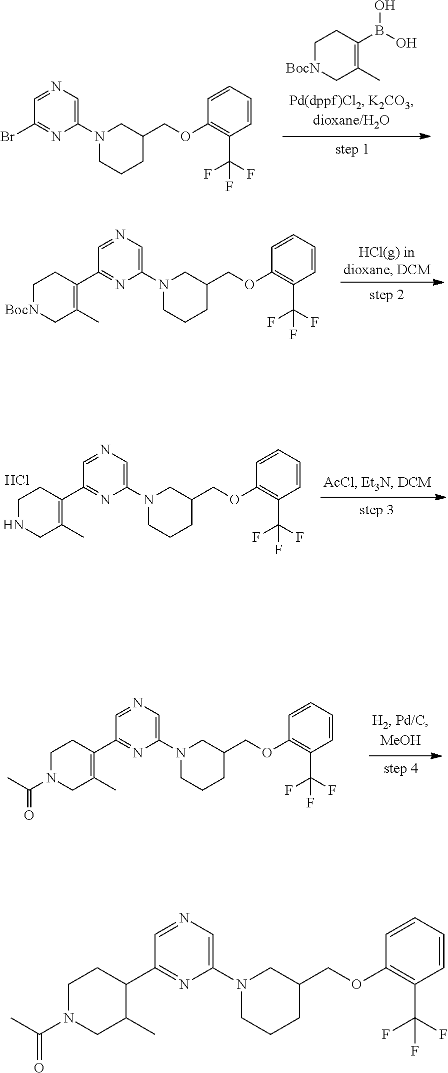

Step 1: tert-butyl 5-methyl-4-(6-(3-((2-(trifluoromethyl)phenoxy)methyl)piperidin-1-yl)pyrazin-2-yl)-3,6-dihydropyridine-1(2H)-carboxylate: A solution of 2-bromo-6-{3-[2-(trifluoromethyl)phenoxymethyl]piperidin-1-yl}pyrazine (130 mg, 0.312 mmol, 1 equiv), Pd(dppf)Cl₂ (22.9 mg, 0.031 mmol, 0.1 equiv), 1-(tert-butoxycarbonyl)-3-methyl-5,6-dihydro-2H-pyridin-4-ylboronic acid (82.8 mg, 0.343 mmol, 1.1 equiv) and K₂CO₃ (86.33 mg, 0.624 mmol, 2 equiv) in dioxane (3 mL)/H₂O (0.6 mL) was stirred for overnight at 80° C. under argon atmosphere. The resulting mixture was concentrated under vacuum. The residue was purified by silica gel column chromatography, eluted with PE/EA (1:1) to afford tert-butyl 5-methyl-4-(6-(3-((2-(trifluoromethyl)phenoxy)methyl)piperidin-1-yl)pyrazin-2-yl)-3,6-dihydropyridine-1(2H)-carboxylate (130 mg, 78.1%). MS m/z: 533 [M+H]⁺.

Step 2: (5-methyl-1,2,3,6-tetrahydropyridin-4-yl)-6-(3-((2-(trifluoromethyl)phenoxy)methyl)pi-peridin-1-yl)pyrazine hydrochloride: A solution of tert-butyl 5-methyl-4-(6-(3-((2-(trifluoromethyl)phenoxy)methyl)piperidin-1-yl)pyrazin-2-yl)-3,6-dihydropyridine-1(2H)-carboxylate (130 mg, 0.244 mmol, 1 equiv) in DCM (3 mL)/HCl (gas) in 1,4-dioxane (3 mL) was stirred for 2 h at room temperature. The resulting mixture was concentrated under vacuum. The crude product (80 mg) was used in the next step directly without further purification. MS m/z: 433 [M+H]⁺.

Step 3: 1-(5-methyl-4-(6-(3-((2-(trifluoromethyl)phenoxy)methyl)piperidin-1-yl)pyrazin-2-yl)-3,6-dihydropyridin-1(2H)-yl)ethan-1-one: A solution of 2-(5-methyl-1,2,3,6-tetrahydropyridin-4-yl)-6-(3-((2-(trifluoromethyl)phenoxy)methyl)piperidin-1-yl)pyrazine (80 mg, 0.185 mmol, 1 equiv), AcCl (29.0 mg, 0.37 mmol, 2 equiv) and Et₃N (56.2 mg, 0.555 mmol, 3 equiv) in DCM (3 mL) was stirred for 2 h at room temperature. The resulting mixture was concentrated under vacuum. The residue was purified by silica gel column chromatography, eluted with PE/EA (1:1) to afford 1-(5-methyl-4-(6-(3-((2-(trifluoromethyl)phenoxy)methyl)piperidin-1-yl)pyrazin-2-yl)-3,6-dihydropyridin-1(2H)-yl)ethan-1-one (75 mg, 85.4%) as a white solid. MS m/z: 475 [M+H]⁺.

Step 4: 1-(3-methyl-4-(6-(3-((2-(trifluoromethyl)phenoxy)methyl)piperidin-1-yl)pyrazin-2-yl)piperidin-1-yl)ethan-1-one: A solution of 1-(5-methyl-4-(6-(3-((2-(trifluoromethyl)phenoxy)methyl)piperidin-1-yl)pyrazin-2-yl)-3,6-dihydropyridin-1(2H)-yl)ethan-1-one (60 mg, 0.126 mmol, 1 equiv) and Pd/C (2.69 mg, 10% Pd on carbon, wetted with water) in CF₃CH₂OH (4 mL) was stirred for 2 days at room temperature under hydrogen atmosphere. The resulting mixture was filtered, the filter cake was washed with MeOH (3×20 mL). The filtrate was concentrated under reduced pressure. The residue was purified by silica gel column chromatography, eluted with PE/EA (1:1) to afford 1-(3-methyl-4-(6-(3-((2-(trifluoromethyl)phenoxy)methyl)piperidin-1-yl)pyrazin-2-yl)piperidin-1-yl)ethan-1-one. The residue was purified by reverse flash chromatography with the following conditions: column, C18 silica gel; mobile phase, MeCN in water, 10% to 100% gradient in 30 min; detector, UV 254 nm. This provided 1-(3-methyl-4-(6-(3-((2-(trifluoromethyl)phenoxy)methyl)piperidin-1-yl)pyrazin-2-yl)piperidin-1-yl)ethan-1-one (15 mg, 24.4%) as a white solid. ¹H NMR (400 MHz, DMSO-d₆) δ 8.10 (s, 1H), 7.67 (d, J=2.0 Hz, 1H), 7.62 (t, J=8.5 Hz, 2H), 7.25 (d, J=8.3 Hz, 1H), 7.09 (t, J=7.6 Hz, 1H), 4.49-4.21 (m, 3H), 4.15-4.06 (m, 1H), 4.04-3.95 (m, 1H), 3.73-3.65 (m, 1H), 3.14-2.88 (m, 4H), 2.28-2.17 (m, 1H), 2.05-1.94 (m, 5H), 1.92-1.72 (m, 3H), 1.70-1.59 (m, 1H), 1.57-1.36 (m, 2H), 0.61-0.47 (m, 3H). MS m/z: 477.2 [M+H]⁺.

### Cyclopropyl(4-(6-(3-((2-(trifluoromethyl)phenoxy)methyl)piperidin-1-yl)pyrazin-2-yl)piperidin-1-yl)methanone (113)

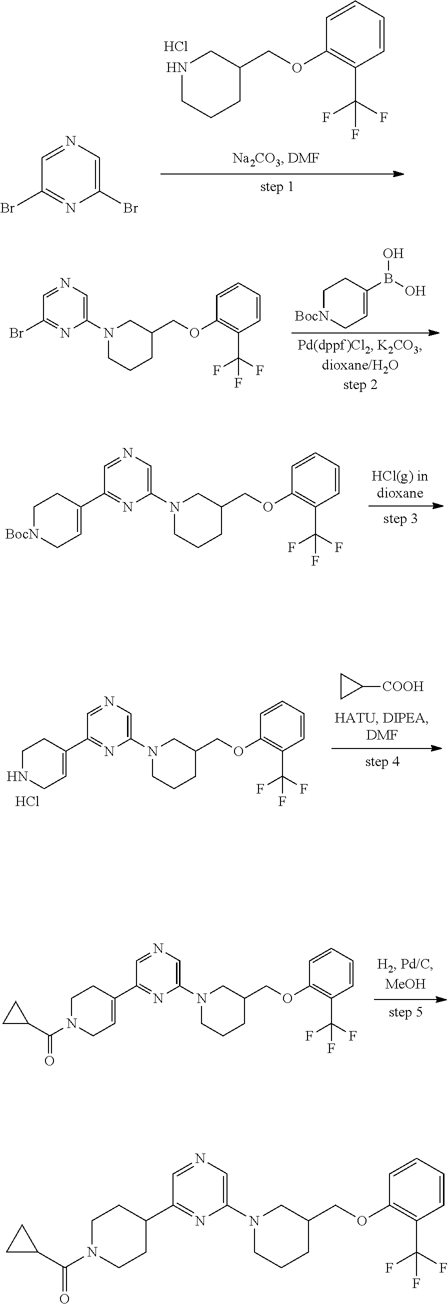

Step 1: 2-bromo-6-(3-((2-(trifluoromethyl)phenoxy)methyl)piperidin-1-yl)pyrazine: A solution of 2,6-dibromopyrazine (300 mg, 1.26 mmol, 1 equiv), 3-[2-(trifluoromethyl)phenoxymethyl]piperidine (359 mg, 1.38 mmol, 1.1 equiv) and Na₂CO₃ (267 mg, 2.52 mmol, 2 equiv) in DMF (4 mL) was stirred for 3 h at 100° C. The resulting mixture was diluted with EtOAc (40 mL). The organic layer was washed with water (3×20 mL), dried over anhydrous Na₂SO₄. After filtration, the filtrate was concentrated under reduced pressure. The residue was purified by silica gel column chromatography, eluted with PE/EA to afford 2-bromo-6-(3-((2-(trifluoromethyl)phenoxy)methyl)piperidin-1-yl)pyrazine (300 mg, 57.1%). MS m/z: 416 [M+H]⁺.

Step 2: tert-butyl 4-(6-(3-((2-(trifluoromethyl)phenoxy)methyl)piperidin-1-yl)pyrazin-2-yl)-3,6-dihydropyridine-1(2H)-carboxylate: A solution of 2-bromo-6-(3-((2-(trifluoromethyl)phenoxy)methyl)piperidin-1-yl)pyrazine (130 mg, 0.312 mmol, 1 equiv), 1-(tert-butoxycarbonyl)-3,6-dihydro-2H-pyridin-4-ylboronic acid (78.0 mg, 0.343 mmol, 1.1 equiv), Pd(dppf)Cl₂ (22.8 mg, 0.031 mmol, 0.1 equiv) and K₂CO₃ (86.3 mg, 0.624 mmol, 2 equiv) in dioxane (5 mL)/H₂O (1 mL) was stirred for 3 h at 80° C. under argon atmosphere. The resulting mixture was concentrated under vacuum. The residue was purified by silica gel column chromatography, eluted with PE/EA to afford tert-butyl 4-(6-(3-((2-(trifluoromethyl)phenoxy)methyl)piperidin-1-yl)pyrazin-2-yl)-3,6-dihydropyridine-1(2H)-carboxylate (150 mg, 92.6%). MS m/z: 519 [M+H]⁺.

Step 3: 2-(1,2,3,6-tetrahydropyridin-4-yl)-6-(3-((2-(trifluoromethyl)phenoxy)methyl)piperidin-1-yl)pyrazine hydrochloride: A solution of tert-butyl 4-(6-(3-((2-(trifluoromethyl)phenoxy)methyl)piperidin-1-yl)pyrazin-2-yl)-3,6-dihydropyridine-1(2H)-carboxylate (150 mg, 0.289 mmol, 1 equiv) in HCl(gas) in 1,4-dioxane (3 mL)/DCM (3 mL) was stirred for 2 h at room temperature. The resulting mixture was concentrated under vacuum. The crude product (100 mg) was used in the next step directly without further purification. MS m/z: 419 [M+H]⁺.

Step 4: 1-(3-methyl-4-(6-(3-((2-(trifluoromethyl)phenoxy)methyl)piperidin-1-yl)pyrazin-2-yl)piperidin-1-yl)ethan-1-one: A solution of 2-(1,2,3,6-tetrahydropyridin-4-yl)-6-(3-((2-(trifluoromethyl)phenoxy)methyl)piperidin-1-yl)pyrazine hydrochloride (100 mg, 0.239 mmol, 1 equiv), cyclopropanecarboxylic acid (24.7 mg, 0.287 mmol, 1.2 equiv), HATU (136 mg, 0.358 mmol, 1.5 equiv) and DIPEA (92.6 mg, 0.717 mmol, 3 equiv) in DMF (2 mL) was stirred for 2 h at room temperature. The resulting mixture was diluted with EtOAc (30 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:1) to afford 1-(3-methyl-4-(6-(3-((2-(trifluoromethyl)phenoxy)methyl)piperidin-1-yl)pyrazin-2-yl)piperidin-1-yl)ethan-1-one (80 mg, 68.1%). MS m/z: 487 [M+H]⁺.

Step 5. cyclopropyl(4-(6-(3-((2-(trifluoromethyl)phenoxy)methyl)piperidin-1-yl)pyrazin-2-yl)piperidin-1-yl)methanone: A solution of 1-(3-methyl-4-(6-(3-((2-(trifluoromethyl)phenoxy)methyl)piperidin-1-yl)pyrazin-2-yl)piperidin-1-yl)ethan-1-one (60 mg, 0.123 mmol, 1 equiv) and Pd/C (1.31 mg, 0.012 mmol, 0.1 equiv) in CF₃CH₂OH (4 mL) was stirred for 1 day at room temperature under hydrogen atmosphere. The resulting mixture was washed with MeOH, and concentrated. The residue was purified by reverse flash chromatography with the following conditions: column, C18 silica gel; mobile phase, MeCN in water, 10% to 100% gradient in 30 min; detector, UV 254 nm. This provided cyclopropyl(4-(6-(3-((2-(trifluoromethyl)phenoxy)methyl)piperidin-1-yl)pyrazin-2-yl)piperidin-1-yl)methanone (30 mg, 48.80%) as an off-white oil. ¹H NMR (400 MHZ, DMSO-d₆) δ 8.10 (s, 1H), 7.73 (s, 1H), 7.61 (t, J=7.2 Hz, 2H), 7.26 (d, J=8.7 Hz, 1H), 7.09 (t, J=7.6 Hz, 1H), 4.54-4.41 (m, 2H), 4.37-4.21 (m, 1H), 4.23-4.15 (m, 1H), 4.10 (dd, J=9.7, 5.1 Hz, 1H), 4.00 (dd, J=9.6, 7.8 Hz, 1H), 3.22-3.08 (m, 1H), 3.00-2.90 (m, 1H), 2.89-2.72 (m, 2H), 2.70-2.61 (m, 1H), 2.08-1.94 (m, 2H), 1.92-1.82 (m, 2H), 1.81-1.71 (m, 2H), 1.64 (s, 1H), 1.55-1.33 (m, 3H), 0.81-0.64 (m, 4H). MS m/z: 498.1 [M+H]⁺.

### 6-((1R,5S,6r)-6-(((3,5-difluoropyridin-4-yl)oxy)methyl)-3-azabicyclo[3.1.0]hexan-3-yl)-1-(oxetan-3-yl)-1H-pyrazolo[3,4-b]pyrazine (114)

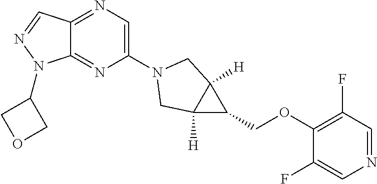 [SMILES: Fc1cncc(F)c1OC[C@@H]1[C@H]2CN(c3cnc4cnn(C5COC5)c4n3)C[C@@H]12]

Step 1: tert-butyl (1R,5S,6r)-6-(((3,5-difluoropyridin-4-yl)oxy)methyl)-3-azabicyclo-[3.1.0]hexane-3-carboxylate: Followed General Procedure D using tert-butyl (1R,5S,6r)-6-(hydroxymethyl)-3-azabicyclo-[3.1.0]hexane-3-carboxylate (220 mg, 1.03 mmol, 1 equiv) and NaH (74.26 mg, 3.1 mmol, 3 equiv) to afford tert-butyl (1R,5S,6r)-6-(((3,5-difluoropyridin-4-yl)oxy)methyl)-3-azabicyclo[3.1.0]hexane-3-carboxylate (220 mg, 65.3%) as a white solid. MS m/z: 327 [M+H]⁺.

Step 2: (1R,5S,6r)-6-(((3,5-difluoropyridin-4-yl)oxy)methyl)-3-azabicyclo[3.1.0]-hexane hydrochloride: Followed General Procedure B using tert-butyl (1R,5S,6r)-6-(((3,5-difluoropyridin-4-yl)oxy)methyl)-3-azabicyclo[3.1.0]hexane-3-carboxylate (220 mg, 0.674 mmol, 1 equiv) to afford the crude product (1R,5S,6r)-6-(((3,5-difluoropyridin-4-yl)oxy)methyl)-3-azabicyclo[3.1.0]hexane hydrochloride(120 mg). MS m/z: 227 [M+H]⁺.

Step 3: 6-((1R,5S,6r)-6-(((3,5-difluoropyridin-4-yl)oxy)methyl)-3-azabicyclo[3.1.0]-hexan-3-yl)-1-(oxetan-3-yl)-1H-pyrazolo[3,4-b]pyrazine: Followed General Procedure C using (1R,5S,6r)-6-(((3,5-difluoropyridin-4-yl)oxy)methyl)-3-azabicyclo-[3.1.0]hexane hydrochloride (120 mg, 0.530 mmol, 1 equiv) and 6-chloro-1-(oxetan-3-yl)-1H-pyrazolo[3,4-b]pyrazine (123 mg, 0.583 mmol, 1.1 equiv) to afford 6-((1R,5S,6r)-6-(((3,5-difluoropyridin-4-yl)oxy)methyl)-3-azabicyclo[3.1.0]hexan-3-yl)-1-(oxetan-3-yl)-1H-pyrazolo[3,4-b]pyrazine (44 mg, 20.7%) as a white solid. ¹H NMR (400 MHz, DMSO-d₆) ¿ 8.51-8.36 (m, 2H), 8.25-8.12 (m, 1H), 8.09-7.97 (m, 1H), 5.96-5.73 (m, 1H), 5.09-5.02 (m, 2H), 5.00-4.91 (m, 2H), 4.35 (d, 2H), 3.92-3.76 (m, 2H), 3.62-3.51 (m, 2H), 1.95-1.78 (m, 2H), 1.20-1.05 (m, 1H). MS m/z: 401.10 [M+H]⁺.

### 5-((3-(((2-(Trifluoromethyl)pyridin-3-yl)oxy)methyl)piperidin-1-yl)sulfonyl)-1H-indole (115)

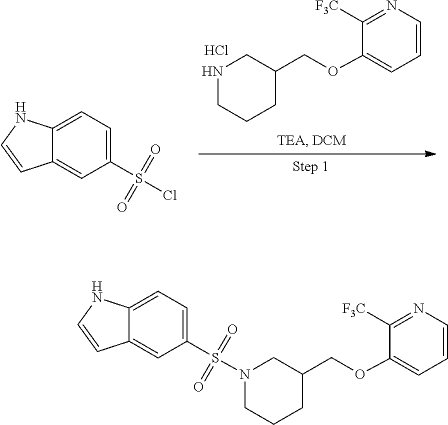 [SMILES: O=S(=O)(Cl)c1ccc2[nH]ccc2c1 ; Cl ; FC(F)(F)c1ncccc1OCC1CCCNC1 ; O=S(=O)(c1ccc2[nH]ccc2c1)N1CCCC(COc2cccnc2C(F)(F)F)C1]

To a stirred solution of 3-(piperidin-3-ylmethoxy)-2-(trifluoromethyl)pyridine hydrochloride (69.7 mg, 0.266 mmol, 1.20 equiv) and 1H-indole-5-sulfonyl chloride (50.0 mg, 0.222 mmol, 1.00 equiv) in DCM (1.00 mL) was added TEA (67.2 mg, 0.666 mmol, 3.00 equiv). The resulting mixture was stirred for 2 h at 0° C. The resulting mixture was concentrated under vacuum. The residue was purified by reverse flash chromatography with the following conditions (column, C18 gel; mobile phase, B phase: MeCN, A phase: water; 0% to 100% B gradient in 20 min; detector: UV 254/220 nm). This provided 5-((3-(((2-(trifluoromethyl)pyridin-3-yl)oxy)methyl)piperidin-1-yl)sulfonyl)-1H-indole (40.0 mg, 41.1%) as a white solid. ¹H NMR (400 MHZ, DMSO-d₆) δ 11.63 (s, 1H), 8.26-8.25 (m, 1H), 7.97-7.96 (m, 1H), 7.76-7.74 (m, 1H), 7.69-7.66 (m, 1H), 7.61-7.54 (m, 2H), 7.42-7.39 (m, 1H), 6.65-6.63 (m, 1H), 4.11-4.07 (m, 1H), 4.01-3.97 (m, 1H), 3.70-3.68 (m, 1H), 3.52-3.50 (m, 1H), 2.33-2.16 (m, 2H), 2.07-2.05 (m, 1H), 1.83-1.64 (m, 2H), 1.56-1.50 (m, 1H), 1.12-0.95 (m, 1H). MS m/z: 440.0 [M+H]⁺.

### 1-(2,2-Difluoroethyl)-6-(2-methyl-3-(((2-(trifluoromethyl)pyridin-3-yl)oxy)methyl)piperidin-1-yl)-1H-pyrazolo[3,4-b]pyrazine (116)

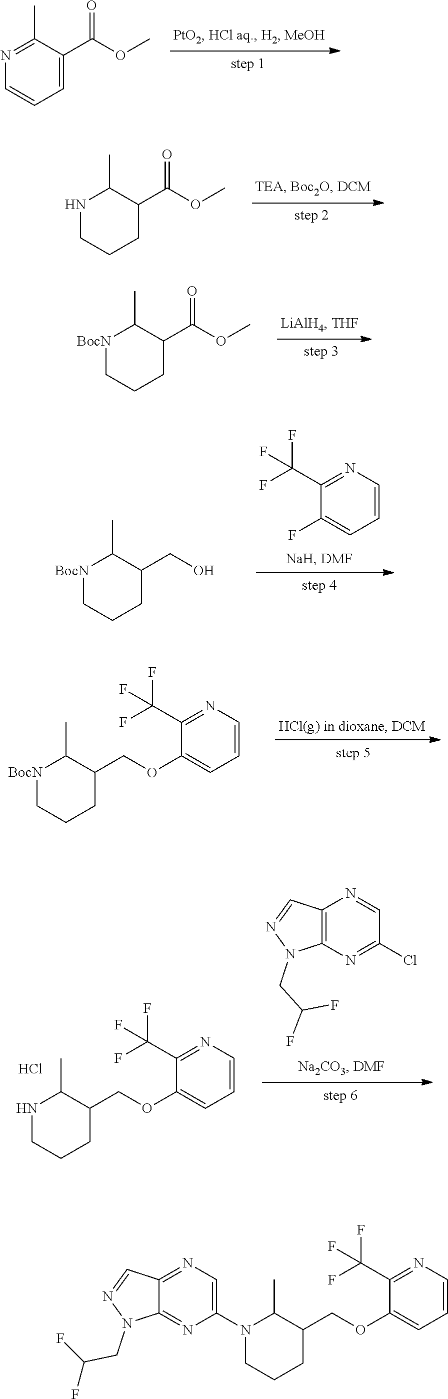

Step 1: methyl 2-methylpiperidine-3-carboxylate: To a stirred mixture of methyl 2-methylnicotinate (1 g, 6.615 mmol, 1 equiv) and PtO₂ (380 mg, 1.673 mmol, 0.25 equiv) in MeOH (5 mL) was added HCl (1 mL) dropwise at room temperature under air atmosphere. The resulting mixture was stirred for overnight at room temperature under hydrogen atmosphere. The resulting mixture was filtered, and the filter cake was washed with MeOH (3×10 mL). The filtrate was concentrated under reduced pressure, the crude product methyl 2-methylpiperidine-3-carboxylate (1.00 g, 96.1%) as a yellow oil. MS m/z: 158 [M+H]⁺.

Step 2: 1-(tert-butyl) 3-methyl 2-methylpiperidine-1,3-dicarboxylate: To a stirred mixture of methyl 2-methylpiperidine-3-carboxylate (1 g, 6.36 mmol, 1 equiv) in DCM (20 mL) was added TEA (2 g, 19.765 mmol, 3.11 equiv) at 0° C. and di-tert-butyl dicarbonate (1 g. 4.58 mmol, 0.72 equiv) at 0° C. under air atmosphere. The resulting mixture was stirred for 2 h at 0° C. under air atmosphere. The resulting mixture was concentrated under reduced pressure. The residue was purified by silica gel column chromatography, eluted with PE/EA (0-100% 20 min) to afford 1-(tert-butyl) 3-methyl 2-methylpiperidine-1,3-dicarboxylate (1.24 g. 75.7%) as a colorless oil. MS m/z: 258 [M+H]⁺.

Step 3: tert-butyl 3-(hydroxymethyl)-2-methylpiperidine-1-carboxylate: To a stirred mixture of 1-(tert-butyl) 3-methyl 2-methylpiperidine-1,3-dicarboxylate (1.2 g. 4.66 mmol, 1 equiv) in tetrahydrofuran (15 mL) was added LiAlH₄ (5.60 mL, 147 mmol, 31.6 equiv) dropwise at 0° C. under air atmosphere. The resulting mixture was stirred for 1 h at 0° C. under air atmosphere. The reaction was quenched by the addition of water (0.24 mL) at 0° C. 10% NaOH (0.48 mL) and water (0.72 mL) sequentially. The resulting mixture was filtered, the filter cake was washed with EtOAc (3×10 mL), dried over anhydrous Na₂SO₄, filtered and concentrated to dryness under vacuum to give the crude product. The residue was purified by silica gel column chromatography, eluted with PE/EA (0-100% 20 min) to afford tert-butyl 3-(hydroxymethyl)-2-methylpiperidine-1-carboxylate (796 mg, 74.4%) as a colorless oil. MS m/z: 230 [M+H]⁺.

Step 4: tert-butyl 2-methyl-3-(((2-(trifluoromethyl)pyridin-3-yl)oxy)methyl)piperidine-1-carboxylate: Followed General Procedure D using tert-butyl 3-(hydroxymethyl)-2-methylpiperidine-1-carboxylate (790 mg, 3.445 mmol, 1 equiv) and 3-fluoro-2-(trifluoromethyl)pyridine (568.73 mg, 3.445 mmol, 1 equiv) to afford tert-butyl 2-methyl-3-(((2-(trifluoromethyl)pyridin-3-yl)oxy)methyl)piperidine-1-carboxylate (1.12 g. 86.83%) as a colorless oil. MS m/z: 375 [M+H]⁺.

Step 5: 3-((2-methylpiperidin-3-yl)methoxy)-2-(trifluoromethyl)pyridine: Followed General Procedure B using tert-butyl 2-methyl-3-(((2-(trifluoromethyl)pyridin-3-yl)oxy)methyl)piperidine-1-carboxylate (1.1 g, 2.938 mmol, 1 equiv) to afford the crude product 3-((2-methylpiperidin-3-yl)methoxy)-2-(trifluoromethyl)pyridine (946 mg, crude) as a white solid. MS m/z: 275 [M+H]⁺.

Step 6: 1-(2,2-difluoroethyl)-6-(2-methyl-3-(((2-(trifluoromethyl)pyridin-3-yl)oxy)methyl)piperidin-1-yl)-1H-pyrazolo[3,4-b]pyrazine: Followed General Procedure C using 3-((2-methylpiperidin-3-yl)methoxy)-2-(trifluoromethyl)pyridine (162.29 mg, 0.522 mmol, 1.1 equiv) and 6-chloro-1-(2,2-difluoroethyl)-1H-pyrazolo[3,4-b]pyrazine (100 mg, 0.475 mmol, 1 equiv. The crude product was purified by reversed phase Combi-flash chromatography with the following conditions (column, C18 gel; mobile phase, B phase: MeCN, A phase: water; 35% to 80% B gradient in 20 min; detector: UV 254/220 nm). The pure fraction was concentrated under vacuum to afford 1-(2,2-difluoroethyl)-6-(2-methyl-3-(((2-(trifluoromethyl)pyridin-3-yl)oxy)methyl)piperidin-1-yl)-1H-pyrazolo[3,4-b]pyrazine (80.6 mg, 33.5%) as a yellow solid. ¹H NMR (400 MHZ, DMSO-d₆) δ 8.40 (s, 1H), 8.28 (s, J=4.6, 1.1 Hz, 1H), 8.13 (s, 1H), 7.87 (d, J=8.5 Hz, 1H), 7.71 (s, J=8.6, 4.5 Hz, 1H), 6.41 (t, J=55.0, 4.0 Hz, 1H), 5.22 (s, 1H), 4.68 (d, J=14.7, 4.0, 2.2 Hz, 2H), 4.37 (d, J=13.5 Hz, 1H), 4.23 (s. J=9.8, 5.9 Hz, 1H), 4.01 (t, J=9.6 Hz, 1H), 3.12 (t, J=12.1 Hz, 1H), 2.28 (s, 1H), 1.89-1.76 (m, 1H), 1.68 (d, J=7.7 Hz, 1H), 1.57 (d, J=11.7, 11.2, 8.1 Hz, 2H), 1.10 (d, J=6.8 Hz, 3H). MS m/z: 457.0 [M+H]⁺.

### 6-(2-Methyl-5-(((2-(trifluoromethyl)pyridin-3-yl)oxy)methyl)piperidin-1-yl)-1-(oxetan-3-yl)-1H-pyrazolo[3,4-b]pyrazine (117)

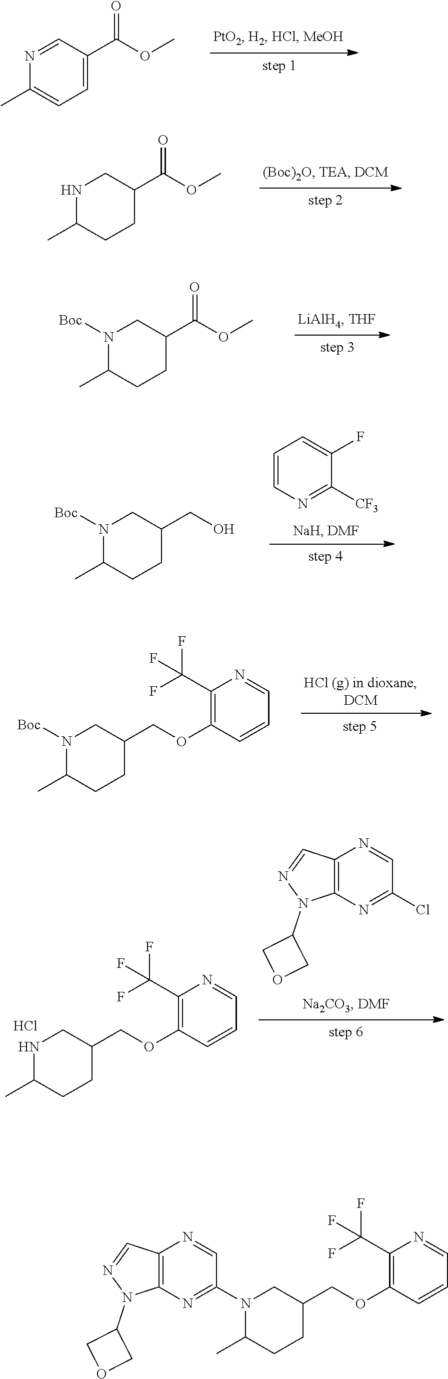

Step 1: methyl 6-methylpiperidine-3-carboxylate: To a stirred mixture of methyl 6-methylnicotinate (1.00 g, 6.61 mmol, 1.00 equiv) and PtO₂ (380 mg, 1.67 mmol, 0.250 equiv) in MeOH (5.00 mL) was added HCl conc. (1.00 mL) dropwise at room temperature under air atmosphere. The resulting mixture was stirred for overnight at room temperature under hydrogen atmosphere. The resulting mixture was filtered, the filter cake was washed with MeOH (3×10 mL). The filtrate was concentrated under reduced pressure, the crude product methyl 6-methylpiperidine-3-carboxylate (1.00 g) was used for next step without further purification. MS m/z: 158 [M+H]⁺.

Step 2: 1-(tert-butyl) 3-methyl 6-methylpiperidine-1,3-dicarboxylate: To a stirred mixture of methyl 6-methylpiperidine-3-carboxylate (1.00 g, 6.36 mmol, 1.00 equiv) in DCM (20.0 mL) was added TEA (2.00 g, 19.7 mmol, 3.11 equiv) at 0° C. and di-tert-butyl dicarbonate (1.00 g, 4.58 mmol, 0.720 equiv) at 0° C. under air atmosphere. The resulting mixture was stirred for 2 h at 0° C. under air atmosphere. The resulting mixture was concentrated under reduced pressure. The residue was purified by silica gel column chromatography, eluted with PE/EtOAc (0-100% 20 min) to afford 1-(tert-butyl) 3-methyl 6-methylpiperidine-1,3-dicarboxylate (1.24 g, 75.7%) as a colorless oil. MS m/z: 258 [M+H]⁺.

Step 3: tert-butyl 5-(hydroxymethyl)-2-methylpiperidine-1-carboxylate: To a stirred mixture of 1-tert-butyl 3-methyl 6-methylpiperidine-1,3-dicarboxylate (1.20 g, 4.66 mmol, 1.00 equiv) in tetrahydrofuran (15.0 mL) was added LiAlH₄ (5.60 mL, 147 mmol, 31.64 equiv) dropwise at 0° C. under air atmosphere. The resulting mixture was stirred for 1 h at 0° C. under air atmosphere. The reaction was quenched by the addition of water (240 μL), 10% NaOH (480 μL) and water (720 μL) sequentially into the mixture. The resulting mixture was filtered, the filter cake was washed with THF (3×10 mL), dried over anhydrous Na₂SO₄, filtered and concentrated to dryness under vacuum to give the crude product. The residue was purified by silica gel column chromatography, eluted with PE/EA (1/1) to afford tert-butyl 5-(hydroxymethyl)-2-methylpiperidine-1-carboxylate (796 mg, 74.4%) as a colorless oil. MS m/z: 230 [M+H]⁺.

Step 4: tert-butyl 2-methyl-5-(((2-(trifluoromethyl)pyridin-3-yl)oxy)methyl)piperidine-1-carboxylate: Followed General Procedure D using tert-butyl 5-(hydroxymethyl)-2-methylpiperidine-1-carboxylate (790 mg, 3.44 mmol, 1.00 equiv) and 3-fluoro-2-(trifluoromethyl)pyridine (568 mg, 3.44 mmol. 1.00 equiv to afford tert-butyl 2-methyl-5-(((2-(trifluoromethyl)pyridin-3-yl)oxy)methyl)piperidine-1-carboxylate (1.12 g, 86.8%) as a colorless oil. MS m/z: 375 [M+H]⁺.

Step 5: 3-((6-methylpiperidin-3-yl)methoxy)-2-(trifluoromethyl)pyridine hydrochloride: Followed General Procedure B using tert-butyl 2-methyl-5-(((2-(trifluoromethyl)pyridin-3-yl)oxy)methyl) piperidine-1-carboxylate (1.10 g, 2.93 mmol, 1.00 equiv) to afford the crude product 3-[(6-methylpiperidin-3-yl)methoxy]-2-(trifluoromethyl)pyridine hydrochloride (946 mg). MS m/z: 311 [M+H]⁺.

Step 6: 6-(2-methyl-5-(((2-(trifluoromethyl)pyridin-3-yl)oxy)methyl)piperidin-1-yl)-1-(oxetan-3-yl)-1H-pyrazolo[3,4-b]pyrazine: Followed General Procedure C using 3-[(6-methylpiperidin-3-yl)methoxy]-2-(trifluoromethyl)pyridine hydrochloride (162.2 mg, 0.522 mmol, 1.10 equiv) and 6-chloro-1-(oxetan-3-yl)-1H-pyrazolo[3,4-b]pyrazine (100 mg, 0.475 mmol, 1.00 equiv). The crude product was purified by reversed phase Combi-flash chromatography with the following conditions (column, C18 gel; mobile phase, B phase: MeCN, A phase: water; 35% to 80% B gradient in 20 min; detector: UV 254/220 nm). The pure fraction was concentrated under vacuum to afford 3-({6-methyl-1-[1-(oxetan-3-yl)pyrazolo[3,4-b]pyrazin-6-yl]piperidin-3-yl}methoxy)-2-(trifluoromethyl)pyridine (50.5 mg, 23.5%) as a yellow solid. ¹H NMR (400 MHZ, DMSO-d₆) δ 8.40 (s, 1H), 8.31-8.28 (m, 1H), 8.18 (s, 1H), 7.85 (d, J=8.5 Hz, 1H), 7.75-7.71 (m, 1H), 5.93-5.82 (m, 1H), 5.06-5.02 (m, 2H), 4.96-4.92 (m, 2H), 4.86 (s, 1H), 4.66 (d, J=13.3 Hz, 1H), 4.29-4.26 (m, 1H), 4.17-4.13 (m, 1H), 2.92 (t, J=12.6 Hz, 1H), 2.06 (d, J=12.9 Hz, 1H), 1.83-1.63 (m, 4H), 1.22 (d, J=6.8 Hz, 3H). MS m/z: 449.2 [M+H]⁺.

### 6-(2-Methyl-5-(((2-(trifluoromethyl)pyridin-3-yl)oxy)methyl)piperidin-1-yl)-1-(oxetan-3-ylmethyl)-1H-pyrazolo[3,4-b]pyrazine (118)

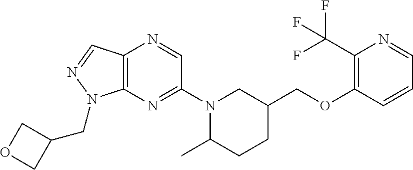 [SMILES: CC1CCC(COc2cccnc2C(F)(F)F)CN1c1cnc2cnn(CC3COC3)c2n1]

Followed General Procedure C using 3-((6-methylpiperidin-3-yl)methoxy)-2-(trifluoromethyl)pyridine hydro-chloride (152 mg, 0.490 mmol, 1.10 equiv) and 6-chloro-1-(oxetan-3-ylmethyl)-1H-pyrazolo[3,4-b]pyrazine (100 mg, 0.445 mmol, 1.00 equiv). The crude product was purified by reversed phase Combi-flash chromatography with the following conditions (column, C18 gel; mobile phase, B phase: MeCN, A phase: water; 30% to 75% B gradient in 20 min; detector: UV 254/220 nm). The pure fraction was concentrated under vacuum to afford 3-({6-methyl-1-[1-(oxetan-3-ylmethyl)pyrazolo[3,4-b]pyrazin-6-yl]piperidin-3-yl}methoxy)-2-(trifluoromethyl)pyridine (102 mg, 47.5%) as a yellow solid. ¹H NMR (400 MHZ, DMSO-d₆) δ 8.38 (s, 1H), 8.29-8.27 (m, 1H), 8.03 (s, 1H), 7.84 (d, J=8.6 Hz, 1H), 7.75-7.70 (m, 1H), 4.86 (s, 1H), 4.70 (d, J=13.5 Hz, 1H), 4.66-4.62 (m, 2H), 4.55-4.45 (m, 4H), 4.30-4.27 (m, 1H), 4.15-4.10 (m, 1H), 3.47-3.39 (m, 1H), 2.91 (t, J=12.7 Hz, 1H), 2.11-2.01 (m, 1H), 1.85-1.61 (m, 4H), 1.22 (d, J=6.7 Hz, 3H). MS m/z: 463.2 [M+H]⁺.

### 1-(2,2-difluoroethyl)-6-(3-(((2-(trifluoromethyl)pyridin-3-yl)oxy)methyl)piperidin-1-yl)-1H-pyrazolo[3,4-d]pyrimidine (119)

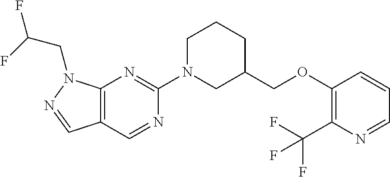 [SMILES: FC(F)Cn1ncc2cnc(N3CCCC(COc4cccnc4C(F)(F)F)C3)nc21]

Step 1: tert-butyl 3-(((2-(trifluoromethyl)pyridin-3-yl)oxy)methyl)piperidine-1-carboxylate: Followed General Procedure D using tert-butyl 3-(hydroxymethyl)piperidine-1-carboxylate (1000 mg, 4.65 mmol, 1 equiv) and 3-fluoro-2-(trifluoromethyl)pyridine (920 mg, 5.58 mmol, 1.2 equiv) to afford tert-butyl 3-(((2-(trifluoromethyl)pyridin-3-yl)oxy)methyl)piperidine-1-carboxylate (1200 mg, 71.6%) as a colorless oil. MS m/z: 361 [M+H]⁺.

Step 2: 3-(piperidin-3-ylmethoxy)-2-(trifluoromethyl)pyridine hydrochloride: Followed General Procedure B using tert-butyl 3-(((2-(trifluoromethyl)pyridin-3-yl)oxy)methyl)piperidine-1-carboxylate (1200 mg, 3.33 mmol, 1 equiv) to afford the crude product 3-(piperidin-3-ylmethoxy)-2-(trifluoromethyl)pyridine hydrochloride (800 mg). MS m/z: 261 [M+H]⁺.

Step 3: 1-(2,2-difluoroethyl)-6-(3-(((2-(trifluoromethyl)pyridin-3-yl)oxy)methyl)piperidin-1-yl)-1H-pyrazolo[3,4-d]pyrimidine: Followed General Procedure C using 3-(piperidin-3-ylmethoxy)-2-(trifluoromethyl)pyridine hydrochloride (80.0 mg, 0.270 mmol, 1.00 equiv) and 6-chloro-1-(2,2-difluoroethyl)-1H-pyrazolo[3,4-d]pyrimidine (58.8 mg, 0.270 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(3-(((2-(trifluoromethyl)pyridin-3-yl)oxy)methyl)piperidin-1-yl)-1H-pyrazolo[3,4-d]pyrimidine (50 mg, 41.8%) as a white solid. ¹H NMR (400 MHZ, DMSO-d₆) δ 8.88 (s, 1H), 8.26 (dd, J=4.6, 1.2 Hz, 1H), 8.05 (s, 1H), 7.80 (d, J=8.6 Hz, 1H), 7.72-7.64 (m, 1H), 6.39 (tt, J=55.1, 4.1 Hz, 1H), 4.92 (d, J=13.0 Hz, 1H), 4.73-4.56 (m, 3H), 4.19 (dd, J=9.8, 4.9 Hz, 1H), 4.05 (t, J=8.9 Hz, 1H), 3.11-3.01 (m, 1H), 2.96 (t, J=11.9 Hz, 1H), 2.12-1.98 (m, 1H), 1.98-1.85 (m, 1H), 1.85-1.73 (m, 1H), 1.53-1.36 (m, 2H). MS m/z: 443.0 [M+H]⁺.

### 6-((1R,5S,6r)-6-(((3,5-difluoropyridin-2-yl)oxy)methyl)-3-azabicyclo[3.1.0]hexan-3-yl)-1-(oxetan-3-yl)-1H-pyrazolo[3,4-b]pyrazine(120)

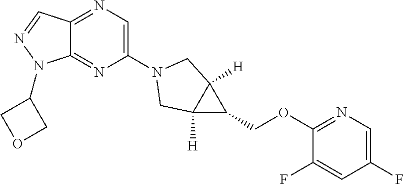 [SMILES: Fc1cnc(OC[C@@H]2[C@H]3CN(c4cnc5cnn(C6COC6)c5n4)C[C@@H]23)c(F)c1]

Step 1: tert-butyl (1R,5S,6R)-6-(((3,5-difluoropyridin-2-yl)oxy)methyl)-3-azabicyclo[3.1.0]hexane-3-carboxylate: Followed General Procedure D using tert-butyl (1R,5S,6r)-6-(hydroxymethyl)-3-azabicyclo[3.1.0]hexane-3-carboxylate (120 mg, 0.563 mmol, 1 equiv) and 2,3,5-trifluoropyridine (89.9 mg, 0.676 mmol, 1.2 equiv) to afford tert-butyl (1R,5S,6r)-6-(((3,5-difluoropyridin-2-yl)oxy)methyl)-3-azabicyclo[3.1.0]hexane-3-carboxylate (130 mg, 74.9%) as a white solid. MS m/z: 327 [M+H]⁺.

Step 2: (1R,5S,6r)-6-(((3,5-difluoropyridin-2-yl)oxy)methyl)-3-azabicyclo[3.1.0]hexane hydrochloride: Followed General Procedure B using tert-butyl (1R,5S,6r)-6-(((3,5-difluoropyridin-2-yl)oxy)methyl)-3-azabicyclo[3.1.0]hexane-3-carboxylate (130 mg, 0.398 mmol, 1 equiv) to afford the crude product (1R,5S,6r)-6-(((3,4-difluoropyridin-2-yl)oxy)methyl)-3-azabicyclo[3.1.0]hexane hydrochloride (50 mg). MS m/z: 227 [M+H]⁺.

Step 3: 6-((1R,5S,6r)-6-(((3,5-difluoropyridin-2-yl)oxy)methyl)-3-azabicyclo[3.1.0]hexan-3-yl)-1-(oxetan-3-yl)-1H-pyrazolo[3,4-b]pyrazine: Followed General Procedure C using (1R,5S,6r)-6-(((3,5-difluoropyridin-2-yl)oxy)methyl)-3-azabicyclo[3.1.0]hexane hydrochloride (50 mg, 0.221 mmol, 1 equiv) and 6-chloro-1-(oxetan-3-yl)-1H-pyrazolo[3,4-b]pyrazine (46.5 mg, 0.221 mmol, 1 equiv). The crude product was purified by reverse flash chromatography with the following conditions: column, C18 silica gel; mobile phase, MeCN in water, 0% to 100% gradient in 30 min; detector, UV 254 nm. This provided 6-((1R,5S,6r)-6-(((3,5-difluoropyridin-2-yl)oxy)methyl)-3-azabicyclo[3.1.0]hexan-3-yl)-1-(oxetan-3-yl)-1H-pyrazolo[3,4-b]pyrazine (42.6 mg, 48.1%) as a white solid. ¹H NMR (400 MHZ, DMSO-d₆) δ 8.18 (s, 1H), 8.10-8.04 (m, 1H), 7.82-7.68 (m, 2H), 5.95-5.80 (m, 1H), 5.11-5.03 (m, 2H), 5.01-4.91 (m, 2H), 4.10 (d, J=7.2 Hz, 2H), 4.02-3.88 (m, 2H), 3.75-3.49 (m, 2H), 1.95-1.85 (m, 2H), 1.28-1.11 (m, 1H). MS m/z: 401.10 [M+H]⁺.

### (1R,5S,6r)-3-(4-(1,3,4-Thiadiazol-2-yl)pyrimidin-2-yl)-6-(((2-(trifluoromethyl)pyridin-3-yl)oxy)methyl)-3-azabicyclo[3.1.0]hexane (121)

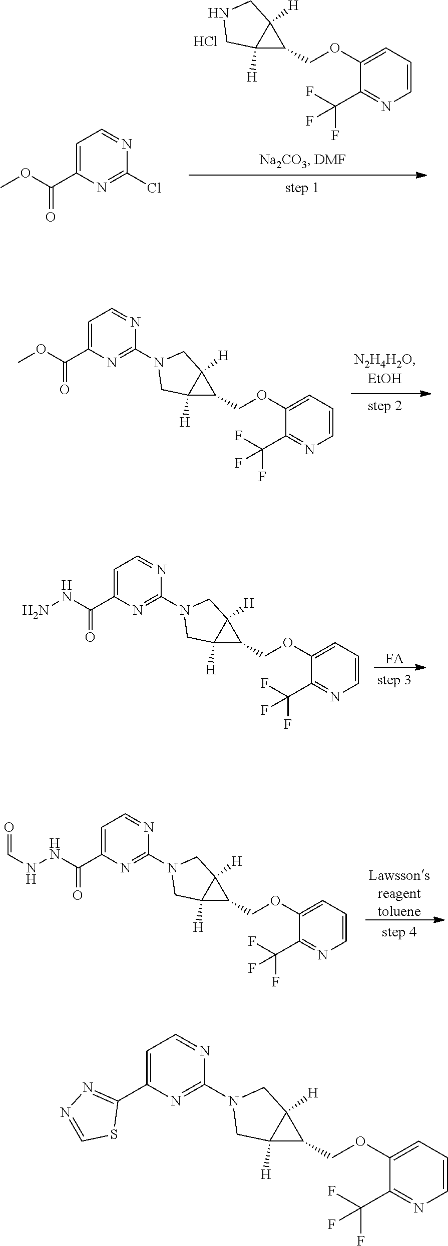

Step 1: methyl 2-((1R,5S,6r)-6-(((2-(trifluoromethyl)pyridin-3-yl)oxy)methyl)-3-azabicyclo[3.1.0]hexan-3-yl)pyrimidine-4-carboxylate: Followed General Procedure C using methyl 2-chloropyrimidine-4-carboxylate (80.0 mg, 0.464 mmol, 1.00 equiv) and (1R,5S,6r)-6-(((2-(trifluoromethyl)pyridin-3-yl)oxy)methyl)-3-azabicyclo[3.1.0]hexane hydrochloride (144 mg, 0.487 mmol, 1.05 equiv) to afford methyl 2-((1R,5S,6r)-6-(((2-(trifluoromethyl)pyridin-3-yl)oxy)methyl)-3-azabicyclo[3.1.0]hexan-3-yl)pyrimidine-4-carboxylate (150 mg, 82.1%) as a light yellow solid. MS m/z: 395 [M+H]⁺.

Step 2: 2-((1R,5S,6r)-6-(((2-(trifluoromethyl)pyridin-3-yl)oxy)methyl)-3-azabicyclo[3.1.0]hexan-3-yl)pyrimidine-4-carbohydrazide: A mixture of methyl 2-((1R,5S,6r)-6-(((2-(trifluoromethyl)pyridin-3-yl)oxy)methyl)-3-azabicyclo[3.1.0]hexan-3-yl)pyrimidine-4-carboxylate (150 mg, 0.380 mmol, 1.00 equiv) and NH₂NH₂H₂O (38.1 mg, 0.760 mmol, 2.00 equiv) in EtOH (3 mL) was stirred for 3 h at 80° C. The resulting mixture was concentrated under vacuum. This provided 2-((1R,5S,6r)-6-(((2-(trifluoromethyl)pyridin-3-yl)oxy)methyl)-3-azabicyclo[3.1.0]hexan-3-yl)pyrimidine-4-carbohydrazide (150 mg) as a light yellow crude solid. MS m/z: 395[M+H]⁺.

Step 3: N′-formyl-2-((1R,5S,6r)-6-(((2-(trifluoromethyl)pyridin-3-yl)oxy)methyl)-3-azabicyclo[3.1.0]hexan-3-yl)pyrimidine-4-carbohydrazide: A solution of 2-((1R,5S,6r)-6-(((2-(trifluoromethyl)pyridin-3-yl)oxy)methyl)-3-azabicyclo[3.1.0]hexan-3-yl)pyrimidine-4-carbohydrazide (150 mg, 0.380 mmol, 1.00 equiv) in HCOOH (3 mL) was stirred for 2 h at 80° C. The residue was purified by reverse flash with the following conditions: column, C18gel; mobile phase, MeCN in water (0.1% FA), 20% to 70% gradient in 16 min; detector, UV 254 nm. This provided N′-formyl-2-((1R,5S,6r)-6-(((2-(trifluoromethyl)pyridin-3-yl)oxy)methyl)-3-azabicyclo[3.1.0]hexan-3-yl)pyrimidine-4-carbohydrazide (160 mg, 99.6%) as a light yellow solid. MS m/z: 423 [M+H]⁺.

Step 4: 2-(2-((1R,5S,6r)-6-(((2-(trifluoromethyl)pyridin-3-yl)oxy)methyl)-3-azabicyclo[3.1.0]hexan-3-yl)pyrimidin-4-yl)-1,3,4-thiadiazole: A mixture of N′-formyl-2-((1R,5S,6r)-6-(((2-(trifluoromethyl)pyridin-3-yl)oxy)methyl)-3-azabicyclo[3.1.0]hexan-3-yl)pyrimidine-4-carbohydrazide (150 mg, 0.355 mmol, 1.00 equiv) and Lawesson's Reagent (143 mg, 0.355 mmol, 1.00 equiv) in toluene (3 mL) was stirred for 2 h at 80° C. The reaction was quenched with sat. NaHCO₃(aq.) at room temperature. The resulting mixture was extracted with EtOAc (3×10 mL). The combined organic layers were dried over anhydrous Na₂SO₄. After filtration, the filtrate was concentrated under reduced pressure. The crude product was purified by Chiral-Prep-HPLC with the following conditions (2 #SHIMADZU (HPLC-01)): Column, Xselect CSH C18 OBD Column 30*150 mm 5 um; mobile phase, water (0.1% FA) and ACN (40% ACN up to 54% in 9 min); Detector, UV 254 nm. This provided 2-(2-((1R,5S,6r)-6-(((2-(trifluoromethyl)pyridin-3-yl)oxy)methyl)-3-azabicyclo[3.1.0]hexan-3-yl)pyrimidin-4-yl)-1,3,4-thiadiazole (84.9 mg, 56.8%) as a light yellow solid. ¹H NMR (300 MHZ, DMSO-d₆): δ 9.76 (s, 1H), 8.58 (d, J=4.8 Hz, 1H), 8.30-8.21 (m, 1H), 7.86-7.77 (m, 1H), 7.73-7.62 (m, 1H), 7.41 (d, J=4.8 Hz, 1H), 4.20 (s, 2H), 3.92-3.80 (m, 2H), 3.65-3.51 (m, 2H), 1.89-1.79 (m, 2H), 1.20-1.10 (m, 1H). MS m/z: 421.1 [M+H]⁺.

### 6-(2-Methyl-5-(((2-(trifluoromethyl)pyridin-3-yl)oxy)methyl)piperidin-1-yl)-1-(2,2,2-trifluoroethyl)-1H-pyrazolo[3,4-b]pyrazine (122)

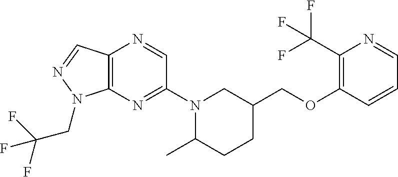 [SMILES: CC1CCC(COc2cccnc2C(F)(F)F)CN1c1cnc2cnn(CC(F)(F)F)c2n1]

Followed General Procedure C using 3-((6-methylpiperidin-3-yl)methoxy)-2-(trifluoromethyl)pyridine hydrochloride (144.4 mg, 0.465 mmol, 1.10 equiv) and 6-chloro-1-(2,2,2-trifluoroethyl)-1H-pyrazolo[3,4-b]pyrazine (100 mg, 0.423 mmol, 1.00 equiv). The crude product was further purified by reversed phase Combi-flash chromatography with the following conditions (column, C18 gel; mobile phase, B phase: MeCN, A phase: water; 30% to 75% B gradient in 20 min; detector: UV 254/220 nm). The pure fraction was concentrated under vacuum to afford 6-(2-methyl-5-(((2-(trifluoromethyl)pyridin-3-yl)oxy)methyl)piperidin-1-yl)-1-(2,2,2-trifluoroethyl)-1H-pyrazolo[3,4-b]pyrazine (83.0 mg, 40.1%) as a light yellow solid. ¹H NMR (300 MHz, DMSO-d₆) δ 8.52 (s, 1H), 8.35 (d, J=4.5 Hz, 1H), 8.25 (s, 1H), 7.92 (d, J=8.5 Hz, 1H), 7.81-7.77 (m, 1H), 5.25-5.10 (m, 2H), 4.90 (d, J=15.0 Hz, 2H), 4.40-4.35 (m, 1H), 4.16 (t, J=8.8 Hz, 1H), 2.95 (t, J=12.6 Hz, −1H), 2.14 (d, J=5.4 Hz, −1H), 1.92-1.65 (m, 4H), 1.30 (d, J=6.7 Hz, 3H). MS m/z: 475.2 [M+H]⁺.

### 1-(2-Methoxyethyl)-6-(2-methyl-5-(((2-(trifluoromethyl)pyridin-3-yl)oxy)methyl)piperidin-1-yl)-1H-pyrazolo[3,4-b]pyrazine (123)

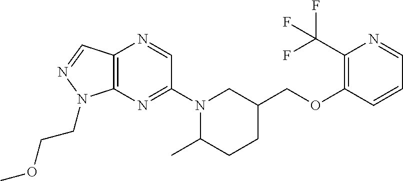 [SMILES: COCCn1ncc2ncc(N3CC(COc4cccnc4C(F)(F)F)CCC3C)nc21]

Followed General Procedure C using 3-((6-methylpiperidin-3-yl)methoxy)-2-(trifluoromethyl)pyridine hydrochloride (160 mg, 0.517 mmol, 1.10 equiv) and 1-(2-methoxyethyl)-6-methyl-1H-pyrazolo[3,4-b]pyrazine (100 mg, 0.470 mmol, 1.00 equiv). The crude product was further purified by reversed phase Combi-flash chromatography with the following conditions (column, C18 gel; mobile phase, B phase: MeCN, A phase: water; 30% to 75% B gradient in 20 min; detector: UV 254/220 nm). The pure fraction was concentrated under vacuum to afford 1-(2-methoxyethyl)-6-(2-methyl-5-(((2-(trifluoromethyl)pyridin-3-yl)oxy)methyl)piperidin-1-yl)-1H-pyrazolo[3,4-b]pyrazine (95.2 mg, 44.8%) as a yellow solid. ¹H NMR (400 MHZ, DMSO-d₆) δ 8.37 (s, 1H), 8.29-8.27 (m, 1H), 8.03 (s, 1H), 7.84 (d, J=8.1 Hz, 1H), 7.73-7.70 (m, 1H), 4.86 (d, J=8.0 Hz, 1H), 4.66 (d, J=12.4 Hz, 1H), 4.42-4.35 (m, 2H), 4.29-4.25 (m, 1H), 4.16-4.12 (m, 1H), 3.77 (t, J=5.5 Hz, 2H), 3.19 (s, 3H), 2.91 (t, J=12.6 Hz, 1H), 2.14-2.01 (m, 1H), 1.85-1.62 (m, 4H), 1.22 (d, J=6.8 Hz, 3H). MS m/z: 451.2 [M+H]⁺.

### 1-Cyclobutyl-6-((1R,5S,6r)-6-((3,5-difluorophenoxy)methyl)-3-azabicyclo[3.1.0]hexan-3-yl)-1H-pyrazolo[3,4-b]pyrazine (124)

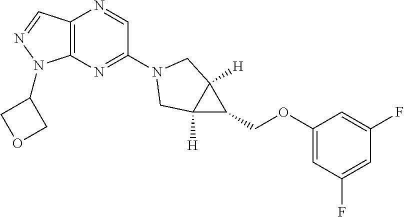 [SMILES: Fc1cc(F)cc(OC[C@@H]2[C@H]3CN(c4cnc5cnn(C6COC6)c5n4)C[C@@H]23)c1]

Followed General Procedure C using (1R,5S,6r)-6-((3,5-difluorophenoxy)methyl)-3-azabicyclo[3.1.0]hexane hydrochloride (50.0 mg, 0.222 mmol, 1.00 equiv) and 6-chloro-1-(oxetan-3-yl)-1H-pyrazolo[3,4-b]pyrazine (51.4 mg, 0.244 mmol, 1.10 equiv). The crude product was purified by reverse flash chromatography with the following conditions (column, C18 gel; mobile phase, B phase: MeCN, A phase: water; 5% to 100% B gradient in 20 min; detector: UV 254/220 nm). This provided 1-cyclobutyl-6-((1R,5S,6r)-6-((3,5-difluorophenoxy)methyl)-3-azabicyclo[3.1.0]hexan-3-yl)-1H-pyrazolo[3,4-b]pyrazine (36.0 mg, 40.3%) as a white solid. ¹H NMR (300 MHz, DMSO-d₆) δ 8.18 (s, 1H), 8.07 (s, 1H), 6.84-6.68 (m, 3H), 5.95-5.80 (m, 1H), 5.06 (t, J=6.4 Hz, 2H), 5.02-4.91 (m, 2H), 3.98 (d, J=7.1 Hz, 2H), 3.91 (d, J=11.0 Hz, 2H), 3.60 (d, J=11.5 Hz, 2H), 1.86 (d, J=3.3 Hz, 2H), 1.19-1.08 (m, 1H). MS m/z: 400.2 [M+H]⁺.

### 4-(2-((1R,5S,6r)-6-((3,5-Difluorophenoxy)methyl)-3-azabicyclo[3.1.0]hexan-3-yl)-2-oxoethyl)-2-phenylpyridazin-3(2H)-one (125)

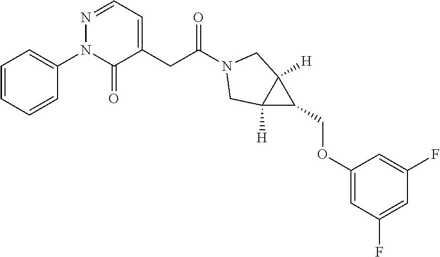 [SMILES: O=C(Cc1ccnn(-c2ccccc2)c1=O)N1C[C@@H]2[C@@H](COc3cc(F)cc(F)c3)[C@@H]2C1]

Followed General Procedure E using 2-(3-oxo-2-phenyl-2,3-dihydropyridazin-4-yl)acetic acid (40 mg, 0.174 mmol, 1.00 equiv) and (1R,5S,6r)-6-((3,5-difluorophenoxy)methyl)-3-azabicyclo[3.1.0]hexane hydrochloride (50.0 mg, 0.191 mmol, 1.10 equiv). The crude product was purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 4-(2-((1R,5S,6r)-6-((3,5-difluorophenoxy)methyl)-3-azabicyclo[3.1.0]hexan-3-yl)-2-oxoethyl)-2-phenylpyridazin-3(2H)-one (28.0 mg, 34.2%) as an orange solid. ¹H NMR (400 MHZ, DMSO-d₆) δ 8.01 (d, J=4.0 Hz, 1H), 7.54-7.47 (m, 4H), 7.44-7.39 (m, 1H), 7.36 (d, J=4.1 Hz, 1H), 6.79-6.67 (m, 3H), 4.00-3.94 (m, 1H), 3.91-3.86 (m, 1H), 3.77 (d, J=10.3 Hz, 1H), 3.69-3.64 (m, 1H), 3.60 (d, J=7.1 Hz, 1H), 3.56 (s, 1H), 3.49 (d, J=15.8 Hz, 1H), 2.83 (s, 1H), 1.76-1.72 (m, 1H), 1.67-1.64 (m, 1H), 1.08-1.02 (m, 1H). MS m/z: 438.2 [M+H]⁺.

### 1-(2-ethoxyethyl)-6-(3-(((2-(trifluoromethyl)pyridin-3-yl)oxy)methyl)piperidin-1-yl)-1H-pyrazolo[3,4-b]pyrazine (126)

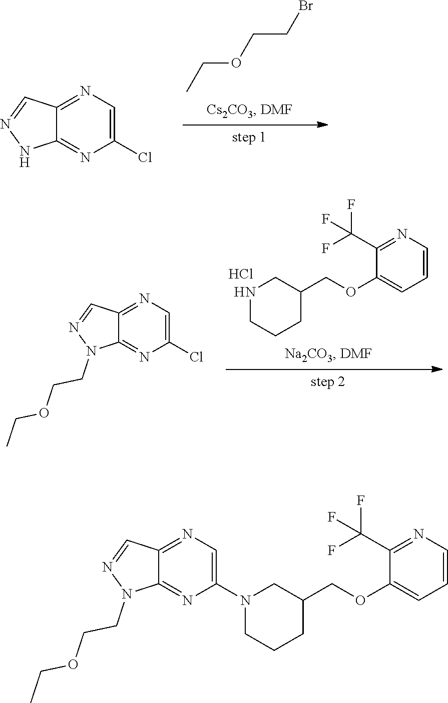

Step 1: 6-chloro-1-(2-ethoxyethyl)-1H-pyrazolo[3,4-b]pyrazine: To a stirred solution of 6-chloro-1H-pyrazolo[3,4-d]pyrimidine (600 mg, 3.87 mmol, 1.00 equiv) and 1-bromo-2-ethoxyethane (706 mg, 4.64 mmol, 1.20 equiv) in DMF (6 mL) was added Cs₂CO₃ (2234 mg, 5.88 mmol, 2.00 equiv). The resulting mixture was stirred for 2 h at room temperature. The reaction mixture was diluted by EtOAc (50 mL), washed by water (2×30 mL) and brine (1×30 mL), dried over anhydrous sodium sulfate. After filtration, the filtrate was concentrated. The residue was purified by silica gel column chromatography, eluted with EtOAc/PE=1/2, to afford 6-chloro-1-(2-ethoxyethyl)-1H-pyrazolo[3,4-b]pyrazine (450 mg, 51.2%). MS m/z: 227 [M+H]⁺.

Step 2: 3-(phenoxymethyl)-1-(1-phenyl-1H-1,2,3-triazol-4-yl)piperidine: Followed General Procedure C using 3-(piperidin-3-ylmethoxy)-2-(trifluoromethyl)pyridine hydrochloride (80.0 mg, 0.270 mmol, 1.00 equiv) and 6-chloro-1-(2-ethoxyethyl)-1H-pyrazolo[3,4-b]pyrazine (61.3 mg, 0.270 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2-ethoxyethyl)-6-(3-(((2-(trifluoromethyl)pyridin-3-yl)oxy)methyl)piperidin-1-yl)-1H-pyrazolo[3,4-b]pyrazine (45 mg, 4.42%) as a white solid. ¹H NMR (300 MHz, DMSO-d₆) δ 8.38 (s, 1H), 8.30-8.23 (m, 1H), 8.03 (s, 1H), 7.82 (d, J=8.6 Hz, 1H), 7.74-7.64 (m, 1H), 4.63 (d, J=13.4 Hz, 1H), 4.40-4.31 (m, 3H), 4.26-4.15 (m, 1H), 4.15-4.04 (m, 1H), 3.78 (t, J=5.7 Hz, 2H), 3.40 (q, J=7.0 Hz, 2H), 3.13 (t, J=11.3 Hz, 1H), 3.00 (dd, J=13.2, 10.2 Hz, 1H), 2.11 (s, 1H), 1.96-1.74 (m, 2H), 1.65-1.36 (m, 2H), 0.97 (t, J=7.0 Hz, 3H). MS m/z: 451.1 [M+H]⁺.

### 6-(3-Methyl-5-(((2-(trifluoromethyl)pyridin-3-yl)oxy)methyl)piperidin-1-yl)-1-(2,2,2-trifluoroethyl)-1H-pyrazolo[3,4-b]pyrazine(127)

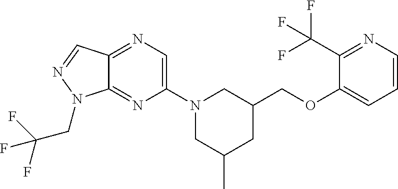 [SMILES: CC1CC(COc2cccnc2C(F)(F)F)CN(c2cnc3cnn(CC(F)(F)F)c3n2)C1]

Followed General Procedure C using 6-chloro-1-(2,2,2-trifluoroethyl)pyrazolo[3,4-b]pyrazine (50 mg, 0.211 mmol, 1 equiv) and 3-[(5-methylpiperidin-3-yl)methoxy]-2-(trifluoromethyl)pyridine (63.8 mg, 0.232 mmol, 1.1 equiv). The crude product was purified by reverse flash chromatography with the following conditions: column, C18 silica gel; mobile phase, MeCN in Water (0.1% FA), 10% to 100% gradient in 20 min; detector, UV 254 nm. This provided 3-({5-methyl-1-[1-(2,2,2-trifluoroethyl)pyrazolo[3,4-b]pyrazin-6-yl]piperidin-3-yl}methoxy)-2-(trifluoromethyl)pyridine (64.5 mg, 64.3%) as a white solid. ¹H NMR (400 MHZ, DMSO-d₆) δ 8.51-8.43 (m, 1H), 8.29-8.22 (m, 1H), 8.19-8.12 (m, 1H), 7.85-7.72 (m, 1H), 7.71-7.61 (m, 1H), 5.21-4.92 (m, 2H), 4.30-3.88 (m, 4H), 3.82-3.75 (m, 1H), 3.49-3.42 (m, 1H), 2.70-2.57 (m, 1H), 2.40-2.30 (m, 1H), 2.19-1.85 (m, 1H), 1.83-1.52 (m, 2H), 1.00-0.93 (m, 3H). MS m/z: 475.1 [M+H]⁺.

### (1H-Indol-6-yl)(2-methyl-5-(((2-(trifluoromethyl)pyridin-3-yl)oxy)methyl)piperidin-1-yl)methanone (128)

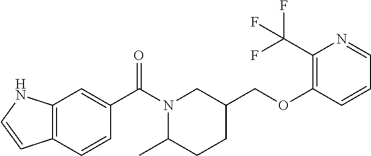 [SMILES: CC1CCC(COc2cccnc2C(F)(F)F)CN1C(=O)c1ccc2cc[nH]c2c1]

Followed General Procedure E using 1H-indole-6-carboxylic acid (60.0 mg, 0.372 mmol, 1.00 equiv) in DMF (2 mL) and 3-((6-methylpiperidin-3-yl)methoxy)-2-(trifluoromethyl)pyridine hydrochloride (127 mg, 0.409 mmol, 1.10 equiv). The crude product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford (1H-indol-6-yl)(2-methyl-5-(((2-(trifluoromethyl) pyridin-3-yl)oxy)methyl)piperidin-1-yl)methanone (93.8 mg, 59.8%) as a white solid. ¹H NMR (400 MHz, DMSO-d₆) δ 11.23 (s, 1H), 8.24 (d, J=4.5 Hz, 1H), 7.81-7.62 (m, 2H), 7.55 (d, J=8.1 Hz, 1H), 7.47-7.38 (m, 2H), 6.98 (d, J=8.1 Hz, 1H), 6.48-6.46 (m, 1H), 4.10 (s, 4H), 2.90 (s, 1H), 2.04 (d, J=29.7 Hz, 1H), 1.80-1.49 (m, 4H), 1.20 (d, J=6.9 Hz, 3H). MS m/z: 418.2 [M+H]⁺.

### (2-(5-Fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (223)

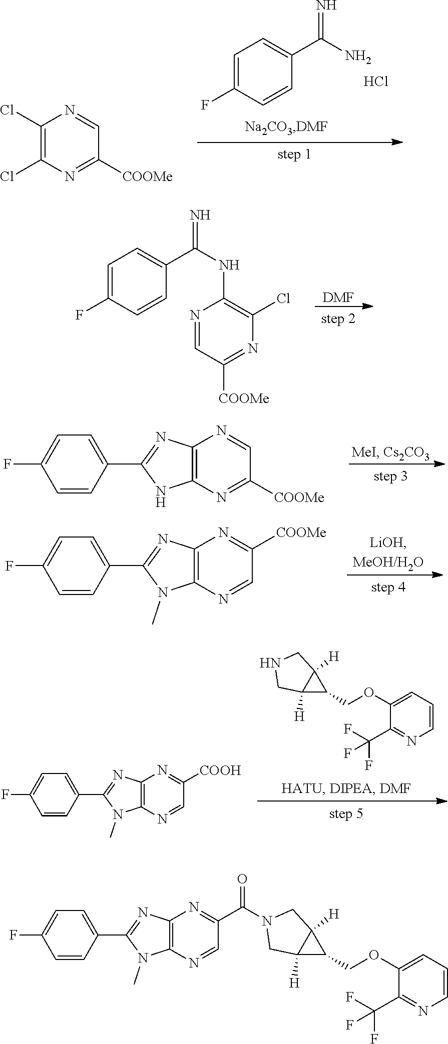

Step 1: methyl 6-chloro-5-(4-fluorobenzimidamido)pyrazine-2-carboxylate: A solution of methyl 5,6-dichloropyrazine-2-carboxylate (1 g, 4.83 mmol, 1 equiv) and Na₂CO₃ (1.54 g, 14.5 mmol, 3 equiv) in DMF (5 mL) was stirred for 2 h at 100° C. The resulting mixture was extracted with EtOAc (30 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:1) to afford methyl 6-chloro-5-(4-fluorobenzeneimidamido)pyrazine-2-carboxylate (1.1 g, 73.8%). MS m/z: 309[M+H]⁺.

Step 2: methyl 2-(4-fluorophenyl)-1H-imidazo[4,5-b]pyrazine-6-carboxylate: A solution of methyl methyl 6-chloro-5-(4-fluorobenzimidamido)pyrazine-2-carboxylate (1.1 g, 3.56 mmol, 1 equiv) in DMF (10 mL) was stirred for overnight at 130° C. The residue was purified by reverse flash chromatography with the following conditions: column, C18 silica gel; mobile phase, MeCN in water, 10% to 100% gradient in 30 min; detector, UV 254 nm. This provided methyl methyl 2-(4-fluorophenyl)-1H-imidazo[4,5-b]pyrazine-6-carboxylate (200 mg, 20.6%). MS m/z: 273[M+H]⁺.

Step 3: methyl 2-(4-fluorophenyl)-1-methyl-1H-imidazo[4,5-b]pyrazine-5-carboxylate: A solution of methyl 2-(4-fluorophenyl)-1H-imidazo[4,5-b]pyrazine-6-carboxylate (200 mg, 0.735 mmol, 1 equiv), MeI (114 mg, 0.808 mmol, 1.1 equiv) and Cs₂CO₃ (478 mg, 1.47 mmol, 2 equiv) in DMF (2 mL) was stirred for overnight at room temperature. The residue was purified by reverse flash chromatography with the following conditions: column, C18 silica gel; mobile phase, MeCN in water, 10% to 100% gradient in 30 min; detector, UV 254 nm. This provided methyl 2-(4-fluorophenyl)-1-methyl-1H-imidazo[4,5-b]pyrazine-5-carboxylate (100 mg, 47.5%). MS m/z: 287[M+H]⁺.

Step 4: 2-(4-fluorophenyl)-1-methyl-1H-imidazo[4,5-b]pyrazine-5-carboxylic acid: A solution of methyl methyl 2-(4-fluorophenyl)-1-methyl-1H-imidazo[4,5-b]pyrazine-5-carboxylate (100 mg, 0.349 mmol, 1 equiv) and LiOH (9.20 mg, 0.384 mmol, 1.1 equiv) in MeOH (0.5 mL)/THF (0.5 mL)/H₂O (0.5 mL) was stirred for overnight at room temperature. The resulting mixture was concentrated under vacuum. The crude product (90 mg) was used in the next step directly without further purification. ES, m/z: 273[M+H]⁺.

Step 5. (2-(4-fluorophenyl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone: A solution of 2-(4-fluorophenyl)-1-methyl-1H-imidazo[4,5-b]pyrazine-5-carboxylic acid (30 mg, 0.110 mmol, 1 equiv), (1R,5S,6S)-6-({[2-(trifluoromethyl)pyridin-3-yl]oxy}methyl)-3-azabicyclo[3.1.0]hexane (31.3 mg, 0.121 mmol, 1.1 equiv), HATU (62.8 mg, 0.165 mmol, 1.5 equiv) and DIPEA (71.2 mg, 0.550 mmol, 5 equiv) in DMF (1 mL) was stirred for 2 h at room temperature. The resulting mixture was diluted with EtOAc (30 mL). The organic layer was washed with water (3×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 30 min; detector, UV 254 nm. This provided (2-(4-fluorophenyl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (8 mg, 14.0%) as a white solid. ¹H NMR (400 MHZ, DMSO-d₆) δ 8.85 (s, 1H), 8.25 (d, J=4.5 Hz, 1H), 8.15-8.05 (m, 2H), 7.80 (d, J=8.6 Hz, 1H), 7.68 (dd, J=8.6, 4.5 Hz, 1H), 7.51 (t. J=8.9 Hz, 2H), 4.22 (dd, J=10.7, 6.6 Hz, 1H), 4.13 (dd, J=10.7, 7.0 Hz, 1H), 4.06-3.88 (m, 6H), 3.61 (dd, J=12.3, 3.9 Hz, 1H), 1.79 (q, J=3.8 Hz, 2H), 1.14 (tt, J=6.8, 3.4 Hz, 1H). MS m/z: 531.1 [M+H]⁺.

### ((2-(4-Fluorophenyl)-1-methyl-1H-imidazo[4,5-b]pyrazin-6-yl)((1R,5S,6r)-6-(((2-(trifluoromethyl)pyridin-3-yl)oxy)methyl)-3-azabicyclo[3.1.0]hexan-3-yl)methanone (224)

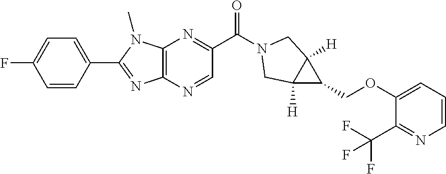 [SMILES: Cn1c(-c2ccc(F)cc2)nc2ncc(C(=O)N3C[C@@H]4[C@@H](COc5cccnc5C(F)(F)F)[C@@H]4C3)nc21]

Followed General Procedure E using 2-(4-fluorophenyl)-3-methylimidazo[4,5-b]pyrazine-5-carboxylic acid (30 mg, 0.110 mmol, 1 equiv) and (1R,5S,6S)-6-({[2-(trifluoromethyl)pyridin-3-yl]oxy}methyl)-3-azabicyclo[3.1.0]hexane (31.3 mg, 0.121 mmol, 1.1 equiv). The crude product was purified by reverse flash chromatography with the following conditions: column, C18 silica gel; mobile phase, MeCN in water, 10% to 100% gradient in 30 min; detector, UV 254 nm. This provided ((2-(4-fluorophenyl)-1-methyl-1H-imidazo[4,5-b]pyrazin-6-yl)((1R,5S,6r)-6-(((2-(trifluoromethyl)pyridine-3-yl)oxy)methyl)-3-azabicyclo[3.1.0]hexan-3-yl)methanone (20 mg, 34.4%) as a white solid. ¹H NMR (400 MHz, DMSO-d₆) δ 8.78 (s, 1H), 8.25 (d, J=4.5 Hz, 1H), 8.14-8.06 (m, 2H), 7.79 (d, J=8.6 Hz, 1H), 7.67 (dd, J=8.6, 4.5 Hz, 1H), 7.55-7.47 (m, 2H), 4.27-4.10 (m, 2H), 3.96 (d, J=14.4 Hz, 6H), 3.61 (dd, J=12.2, 3.9 Hz, 1H), 1.83-1.74 (m, 2H), 1.13 (tt, J=6.8, 3.3 Hz, 1H). MS m/z: 513.1 [M+H]⁺.

### 1-(4-(5-Methyl-2-((1R,5S,6r)-6-(((6-(trifluoromethyl)pyridin-2-yl)oxy)methyl)-3-azabicyclo[3.1.0]hexane-3-carbonyl)-5H-pyrrolo[2,3-b]pyrazin-6-yl)piperidin-1-yl)ethan-1-one (225)

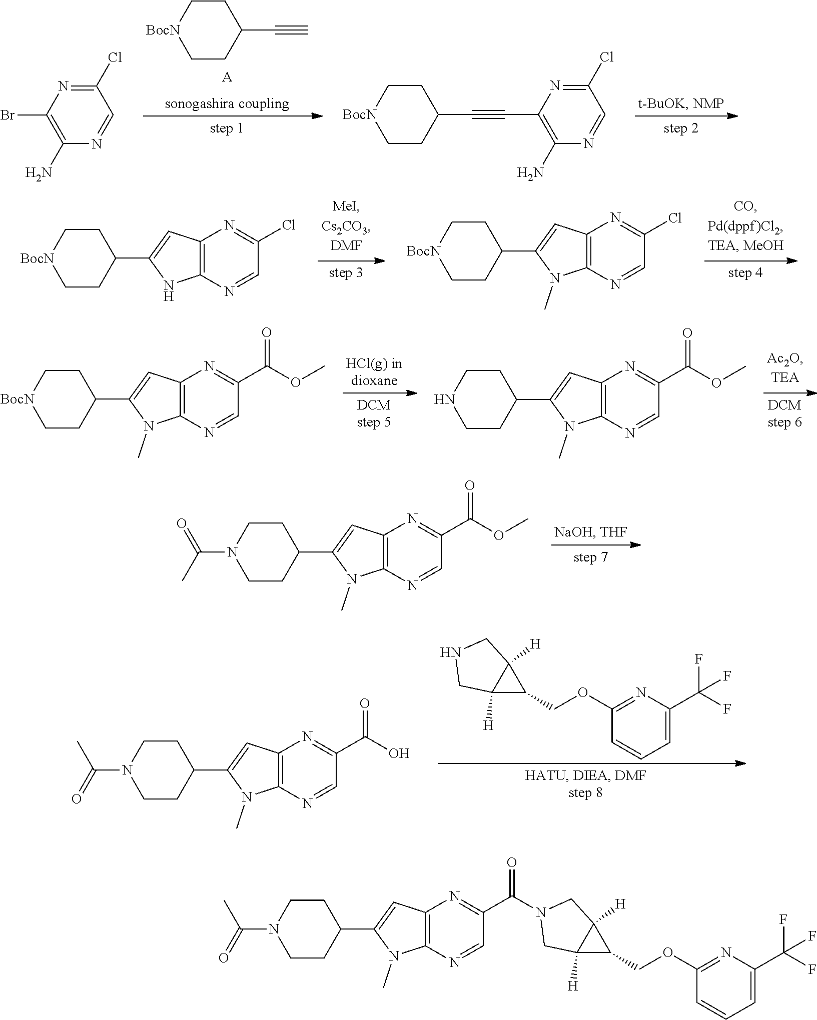

Step 1: tert-butyl 4-((3-amino-6-chloropyrazin-2-yl)ethynyl)piperidine-1-carboxylate: To a stirred solution of 3-bromo-5-chloropyrazin-2-amine (2 g, 9.60 mmol, 1 equiv) and tert-butyl 4-ethynylpiperidine-1-carboxylate (2.41 g, 11.5 mmol, 1.2 equiv) in THF (30 mL) was added CuI (0.18 g, 0.960 mmol, 0.1 equiv) and Pd(PPh₃)₂Cl₂ (0.67 g, 0.960 mmol, 0.1 equiv) and TEA (2.91 g, 28.8 mmol, 3.0 equiv). The resulting mixture was stirred for 2 h at 80° C. The reaction was monitored by LCMS. Desired product could be detected by LCMS. The resulting mixture was concentrated and purified by reverse flash chromatography with the following conditions: column, C18 silica gel; mobile phase, ACN in water, 5% to 100% gradient in 20 min; detector, UV 254/220 nm to afford tert-butyl 4-((3-amino-6-chloropyrazin-2-yl)ethynyl)piperidine-1-carboxylate (2.00 g, 62.5%) as a white solid. MS m/z: 337 [M+H]⁺.

Step 2: tert-butyl 4-(2-chloro-5H-pyrrolo[2,3-b]pyrazin-6-yl)piperidine-1-carboxylate: To a stirred solution of tert-butyl 4-((3-amino-6-chloropyrazin-2-yl)ethynyl)piperidine-1-carboxylate (1 g, 3.00 mmol, 1 equiv) and t-BuOK (0.67 g, 5.94 mmol, 2 equiv) in NMP (10 mL). The resulting mixture was stirred for 2 h at 80° C. The reaction was monitored by LCMS. Desired product could be detected by LCMS. The resulting mixture was purified by reverse flash chromatography with the following conditions: column, C18 silica gel; mobile phase, ACN in water, 5% to 100% gradient in 20 min; detector, UV 254/220 nm to afford tert-butyl 4-(2-chloro-5H-pyrrolo[2,3-b]pyrazin-6-yl)piperidine-1-carboxylate (600 mg, 60.0%) as a white solid. MS m/z: 337 [M+H]⁺.

Step 3: tert-butyl 4-(2-chloro-5-methyl-5H-pyrrolo[2,3-b]pyrazin-6-yl)piperidine-1-carboxylate: To a stirred solution of tert-butyl 4-(2-chloro-5H-pyrrolo[2,3-b]pyrazin-6-yl)piperidine-1-carboxylate (1 g, 2.97 mmol, 1 equiv) and Cs₂CO₃ (2.90 g, 8.91 mmol, 3.0 equiv) and MeI (0.51 g, 3.56 mmol, 1.2 equiv) in DMF (15 mL). The resulting mixture was stirred for 2 h at room temperature. The reaction was monitored by LCMS. Desired product could be detected by LCMS. The resulting mixture was purified by reverse flash chromatography with the following conditions: column, C18 silica gel; mobile phase, ACN in water, 5% to 100% gradient in 20 min; detector, UV 254/220 nm to afford tert-butyl 4-(2-chloro-5-methyl-5H-pyrrolo[2,3-b]pyrazin-6-yl)piperidine-1-carboxylate (500 mg, 48.0%) as a white solid. MS m/z: 351 [M+H]⁺.

Step 4: methyl 6-(1-(tert-butoxycarbonyl)piperidin-4-yl)-5-methyl-5H-pyrrolo[2,3-b]pyrazine-2-carboxylate: To a stirred solution of tert-butyl 4-(2-chloro-5-methyl-5H-pyrrolo[2,3-b]pyrazin-6-yl)piperidine-1-carboxylate (500 mg, 1.42 mmol, 1 equiv) and Pd(dppf)Cl₂ (104 mg, 0.143 mmol, 0.1 equiv) and TEA (432 mg, 4.27 mmol, 3.0 equiv) in MeOH (5 mL). The resulting mixture was stirred for 16 h at 100° C. at CO atmosphere (50 atm). The reaction was monitored by LCMS. Desired product could be detected by LCMS. The resulting mixture was purified by reverse flash chromatography with the following conditions: column, C18 silica gel; mobile phase, ACN in water, 5% to 100% gradient in 20 min; detector, UV 254/220 nm to afford methyl 6-(1-(tert-butoxycarbonyl)piperidin-4-yl)-5-methyl-5H-pyrrolo[2,3-b]pyrazine-2-carboxylate as a white solid. MS m/z: 375[M+H]⁺.

Step 5: methyl 5-methyl-6-(piperidin-4-yl)-5H-pyrrolo[2,3-b]pyrazine-2-carboxylate hydrochloride: To a stirred solution of methyl 6-(1-(tert-butoxycarbonyl)piperidin-4-yl)-5-methyl-5H-pyrrolo[2,3-b]pyrazine-2-carboxylate (200 mg, 0.534 mmol, 1 equiv) in DCM (5 mL) and HCl(gas) in 1,4-dioxane (5 mL). The resulting mixture was stirred for 2 h at room temperature. The resulting mixture was concentrated under vacuum. This provided methyl 5-methyl-6-(piperidin-4-yl)-5H-pyrrolo[2,3-b]pyrazine-2-carboxylate hydrochloride (200 mg, crude) as a white solid. MS m/z: 275 [M+H]⁺.

Step 6: methyl 6-(1-acetylpiperidin-4-yl)-5-methyl-5H-pyrrolo[2,3-b]pyrazine-2-carboxylate: To a stirred solution of methyl 5-methyl-6-(piperidin-4-yl)-5H-pyrrolo[2,3-b]pyrazine-2-carboxylate hydrochloride (300 mg, 1.094 mmol, 1 equiv) and TEA (332 mg, 3.28 mmol, 3.0 equiv) and Ac₂O (134 mg, 1.31 mmol, 1.2 equiv) in DCM (4 mL). The resulting mixture was stirred for 2 h at room temperature The reaction was monitored by LCMS. Desired product could be detected by LCMS. The resulting mixture was concentrated and purified by reverse flash chromatography with the following conditions: column, C18 silica gel; mobile phase, ACN in water, 5% to 100% gradient in 20 min; detector, UV 254/220 nm to afford methyl 6-(1-acetylpiperidin-4-yl)-5-methyl-5H-pyrrolo[2,3-b]pyrazine-2-carboxylate as a white solid. MS m/z: 317 [M+H]⁺.

Step 7: 6-(1-acetylpiperidin-4-yl)-5-methyl-5H-pyrrolo[2,3-b]pyrazine-2-carboxylic acid: To a stirred solution of methyl 6-(1-acetylpiperidin-4-yl)-5-methyl-5H-pyrrolo[2,3-b]pyrazine-2-carboxylate (118 mg, 0.373 mmol, 1 equiv) and NaOH (60 mg, 1.5 mmol, 4 equiv) in MeOH (1 mL) and H₂O (1 mL). The resulting mixture was stirred for 2 h at room temperature The reaction was monitored by LCMS. Desired product could be detected by LCMS. The residue was purified by reverse flash chromatography with the following conditions: column, C18 silica gel; mobile phase, ACN in water, 5% to 50% gradient in 20 min; detector, UV 254/220 nm to afford 6-(1-acetylpiperidin-4-yl)-5-methyl-5H-pyrrolo[2,3-b]pyrazine-2-carboxylic acid as a white solid. MS m/z: 303 [M+H]⁺.

Step 8: 1-(4-(5-methyl-2-((1R,5S,6r)-6-(((6-(trifluoromethyl)pyridin-2-yl)oxy)methyl)-3-azabicyclo[3.1.0]hexane-3-carbonyl)-5H-pyrrolo[2,3-b]pyrazin-6-yl)piperidin-1-yl)ethan-1-one: To a stirred solution of 6-(1-acetylpiperidin-4-yl)-5-methyl-5H-pyrrolo[2,3-b]pyrazine-2-carboxylic acid (50 mg, 0.16 mmol, 1 equiv) and (1R,5S,6r)-6-(((6-(trifluoromethyl)pyridin-2-yl)oxy)methyl)-3-azabicyclo[3.1.0]hexane (47 mg, 0.18 mmol, 1.1 equiv) HATU (69 mg, 0.18 mmol, 1.1 equiv) and DIPEA (64 mg, 0.49 mmol, 3.0 equiv) in DMF (1.0 mL). The resulting mixture was stirred for 2 h at room temperature. The reaction was monitored by LCMS. Desired product could be detected by LCMS. The residue was purified by reverse flash chromatography with the following conditions: column, C18 silica gel; mobile phase, ACN in water, 5% to 100% gradient in 20 min; detector, UV 254/220 nm to afford 1-(4-(5-methyl-2-((1R,5S,6r)-6-(((6-(trifluoromethyl)pyridin-2-yl)oxy)methyl)-3-azabicyclo[3.1.0]hexane-3-carbonyl)-5H-pyrrolo[2,3-b]pyrazin-6-yl)piperidin-1-yl)ethan-1-one (13.6 mg, 15.0%) as a yellow solid. ¹H NMR (300 MHZ, DMSO-d₆) δ 8.60 (s, 1H), 8.03 (dd, 1H), 7.53 (d, J=7.3 Hz, 1H), 7.22 (d, J=8.4 Hz, 1H), 6.63 (s, 1H), 4.62 (d, J=12.7 Hz, 1H), 4.28 (d, J=7.2 Hz, 2H), 4.10-3.98 (m, 3H), 3.92 (s, 4H), 3.61 (d, J=12.4 Hz, 1H), 3.34-3.25 (m, 2H), 2.85-2.72 (m, 1H), 2.15-2.03 (m, 5H), 1.84 (s, 2H), 1.78-1.51 (m, 2H), 1.23-1.09 (m, 1H). MS m/z: 543.2 [M+H]⁺.

### (1R,5S,6S)-3-[6-(1,3,4-thiadiazol-2-yl)pyrazine-2-carbonyl]-6-({[6-(trifluoromethyl)pyridin-2-yl]oxy}methyl)-3-azabicyclo[3.1.0]hexane (226)

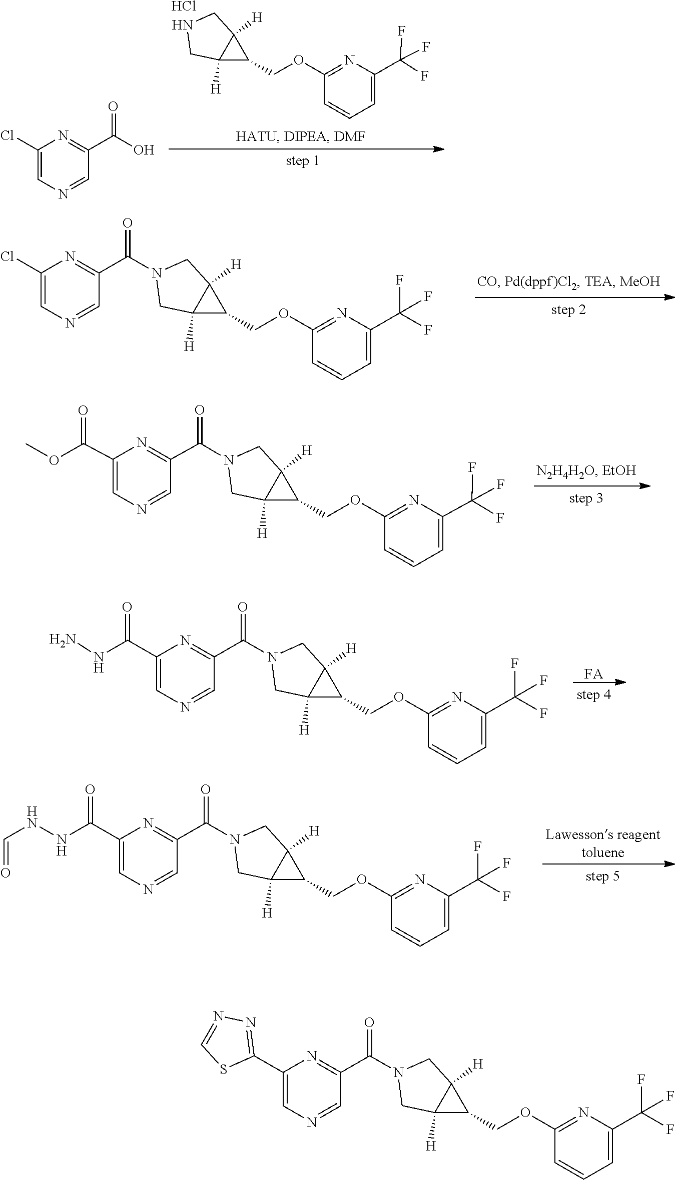

Step 1: (6-chloropyrazin-2-yl)((1R,5S,6r)-6-(((6-(trifluoromethyl)pyridin-2-yl)oxy)methyl)-3-azabicyclo[3.1.0]hexan-3-yl)methanone: To a stirred mixture of 6-chloropyrazine-2-carboxylic acid (120 mg, 0.757 mmol, 1.00 equiv) and (1R,5S,6r)-6-(((6-(trifluoromethyl)pyridin-2-yl)oxy)methyl)-3-azabicyclo[3.1.0]hexane (223 mg, 0.757 mmol, 1.00 equiv) in DMF (3 mL) were added DIPEA (391 mg, 3.03 mmol, 4.00 equiv) and HATU (317 mg, 0.833 mmol, 1.10 equiv) at 0° C. The resulting mixture was stirred for 2 h at room temperature. The resulting mixture was diluted with water (10 mL). The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (2:1) to afford (6-chloropyrazin-2-yl)((1R,5S,6r)-6-(((6-(trifluoromethyl)pyridin-2-yl)oxy)methyl)-3-azabicyclo[3.1.0]hexan-3-yl)methanone (270 mg, 89.5%) as a light yellow oil. MS m/z: 399 [M+H]⁺.

Step 2: methyl 6-((1R,5S,6r)-6-(((6-(trifluoromethyl)pyridin-2-yl)oxy)methyl)-3-azabicyclo[3.1.0]hexane-3-carbonyl)pyrazine-2-carboxylate: To a stirred solution of (6-chloropyrazin-2-yl)((1R,5S,6r)-6-(((6-(trifluoromethyl)pyridin-2-yl)oxy)methyl)-3-azabicyclo[3.1.0]hexan-3-yl)methanone (270 mg, 0.677 mmol, 1.00 equiv) in MeOH (10 mL) were added Pd(dppf)Cl₂ (49.54 mg, 0.068 mmol, 0.10 equiv) and TEA (206 mg, 2.03 mmol, 3.00 equiv) at 0° C. The resulting mixture was stirred for overnight at 100° C. under carbon monoxide atmosphere (30 atm). The resulting mixture was diluted with water (30 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (1:1) to afford methyl 6-((1R,5S,6r)-6-(((6-(trifluoromethyl)pyridin-2-yl)oxy)methyl)-3-azabicyclo[3.1.0]hexane-3-carbonyl)pyrazine-2-carboxylate (220 mg, 76.9%) as a light yellow oil. MS m/z: 423 [M+H]⁺.

Step 3: 6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide: A mixture of 6-((1R,5S,6r)-6-(((6-(trifluoromethyl)pyridin-2-yl)oxy)methyl)-3-azabicyclo[3.1.0]hexane-3-carbonyl)pyrazine-2-carboxylate (140 mg, 0.331 mmol, 1.00 equiv) and NH₂NH₂H₂O (47.4 mg, 0.948 mmol, 2.00 equiv) in EtOH (3 mL) was stirred for 2 h at 80° C. The resulting mixture was concentrated under vacuum. This provided 6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide (160 mg) as a light yellow crude solid. MS m/z: 423 [M+H]⁺.

Step 4: N′-formyl-6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide: A solution of 6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide (160 mg, 0.379 mmol, 1.00 equiv) in HCOOH (3 mL) was stirred for 2 h at 80° C. The resulting mixture was concentrated under vacuum. The residue was purified by reverse flash with the following conditions: column, C18 gel; mobile phase, MeCN in water (0.1% FA), 20% to 60% gradient in 16 min; detector, UV 254 nm. This provided N′-formyl-6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide (30 mg, 17.6%) as a light yellow solid. MS m/z: 451 [M+H]⁺.

Step 5: (6-(1,3,4-thiadiazol-2-yl)pyrazin-2-yl)((1R,5S,6r)-6-(((6-(trifluoromethyl)pyridin-2-yl)oxy)methyl)-3-azabicyclo[3.1.0]hexan-3-yl)methanone: To a stirred solution of N′-formyl-6-((1R,5S,6r)-6-(((6-(trifluoromethyl)pyridin-2-yl)oxy)methyl)-3-azabicyclo[3.1.0]hexane-3-carbonyl)pyrazine-2-carbohydrazide (30 mg, 0.067 mmol, 1.00 equiv) in toluene (2 mL) was added Lawesson reagent (21.6 mg, 0.054 mmol, 0.800 equiv). The resulting mixture was stirred for 2 h at 80° C. The reaction was quenched with sat. NaHCO₃(aq.) at 0° C. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse flash with the following conditions: column, C18 gel; mobile phase, MeCN in water (0.1% FA), 30% to 60% gradient in 16 min; detector, UV 254 nm. This provided (6-(1,3,4-thiadiazol-2-yl)pyrazin-2-yl)((1R,5S,6r)-6-(((6-(trifluoromethyl)pyridin-2-yl)oxy)methyl)-3-azabicyclo[3.1.0]hexan-3-yl)methanone (10.3 mg, 34.4%) as a light yellow solid. ¹H NMR (400 MHZ, CD₃OD): δ 9.62 (s, 1H), 9.58 (s, 1H), 9.10 (s, 1H), 7.84 (t, J=8.0 Hz, 1H), 7.32 (d, J=7.2 Hz, 1H), 7.00 (d, J=8.0 Hz, 1H), 4.35-4.25 (m, 2H), 4.18-3.98 (m, 3H), 3.70 (dd, J=12.0, 4.0 Hz, 1H), 1.91-1.80 (m, 2H), 1.24-1.16 (m, 1H). MS m/z: 449.0 [M+H]⁺.

### 5-((1R,5S,6r)-6-((3,5-difluorophenoxy)methyl)-3-azabicyclo[3.1.0]hexan-3-yl)-1-methyl-2-(6-(trifluoromethyl)pyridin-2-yl)-1H-imidazo[4,5-b]pyrazine (227)

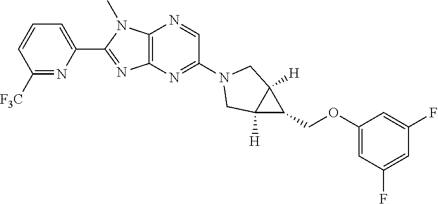 [SMILES: Cn1c(-c2cccc(C(F)(F)F)n2)nc2nc(N3C[C@@H]4[C@@H](COc5cc(F)cc(F)c5)[C@@H]4C3)cnc21]

Followed General Procedure C using (1R,5S,6R)-6-(3,5-difluorophenoxymethyl)-3-azabicyclo[3.1.0]hexane (37.74 mg, 0.168 mmol, 1 equiv) and 5-bromo-1-methyl-2-(6-(trifluoromethyl)pyridin-2-yl)-1H-imidazo[4,5-b]pyrazine (60 mg, 0.168 mmol, 1 equiv). The crude product was initially purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford (1R,5S,6S)-6-(3,5-difluorophenoxymethyl)-3-{3-methyl-2-[6-(trifluoromethyl)pyridin-2-yl]imidazo[4,5-b]pyrazin-5-yl}-3-azabicyclo[3.1.0]hexane (28.7 mg, 33.75%) as a yellow green solid. ¹H NMR (400 MHZ, DMSO-d₆) δ 8.62 (d, J=8.1 Hz, 1H), 8.28 (t, J=7.8 Hz, 1H), 8.10 (d, J=4.2 Hz, 2H), 8.00 (s, J=3.9 Hz, 1H), 6.81-6.70 (m, 3H), 4.21 (s, 3H), 3.99 (d, J=7.2 Hz, 2H), 3.91 (d, J=10.5 Hz, 2H), 3.56 (d, J=10.1 Hz, 2H), 1.88 (s, 2H), 1.27 (t, J=3.5 Hz, 1H). MS m/z:503.2 [M+H]⁺.

### 6-((1R,5S,6r)-6-(((3,4-Difluoropyridin-2-yl)oxy)methyl)-3-azabicyclo[3.1.0]hexan-3-yl)-1-(oxetan-3-yl)-1H-pyrazolo[3,4-b]pyrazine (278)

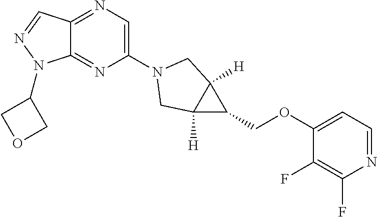 [SMILES: Fc1nccc(OC[C@@H]2[C@H]3CN(c4cnc5cnn(C6COC6)c5n4)C[C@@H]23)c1F]

Step 1: tert-butyl (1R,5S,6r)-6-(((2,3-difluoropyridin-4-yl)oxy)methyl)-3-azabicyclo[3.1.0]hexane-3-carboxylate: Followed General Procedure D using tert-butyl (1R,5S,6r)-6-(hydroxymethyl)-3-azabicyclo[3.1.0]hexane-3-carboxylate (120 mg, 0.563 mmol, 1 equiv) and 2,3,4-trifluoropyridine (89.9 mg, 0.676 mmol, 1.2 equiv) to afford tert-butyl (1R,5S,6r)-6-(((2,3-difluoropyridin-4-yl)oxy)methyl)-3-azabicyclo[3.1.0]hexane-3-carboxylate (148 mg, 80.6%) as a white solid. MS m/z: 327 [M+H]⁺.

Step 2: (1R,5S,6r)-6-(((2,3-difluoropyridin-4-yl)oxy)methyl)-3-azabicyclo[3.1.0]he-xane hydrochloride: Followed General Procedure B using tert-butyl (1R,5S,6r)-6-(((2,3-difluoropyridin-4-yl)oxy)methyl)-3-azabicyclo[3.1.0]hexane-3-carboxylate (148 mg, 0.454 mmol, 1 equiv) to afford the crude product (1R,5S,6r)-6-(((2,3-difluoropyridin-4-yl)oxy)methyl)-3-azabicyclo[3.1.0]hexane hydrochloride (60 mg). MS m/z: 227 [M+H]⁺.

Step 3: 6-((1R,5S,6r)-6-(((2,3-difluoropyridin-4-yl)oxy)methyl)-3-azabicyclo[3.1.0]-hexan-3-yl)-1-(oxetan-3-yl)-1H-pyrazolo[3,4-b]pyrazine: Followed General Procedure C using (1R,5S,6r)-6-(((2,3-difluoropyridin-4-yl)oxy)methyl)-3-azabicyclo-[3.1.0]hexane hydrochloride (60 mg, 0.265 mmol, 1 equiv) and 6-chloro-1-(oxetan-3-yl)-1H-pyrazolo[3,4-b]pyrazine (55.9 mg, 0.265 mmol, 1 equiv). The crude product was purified by reverse flash chromatography with the following conditions: column, C18 silica gel; mobile phase, MeCN in water, 0% to 100% gradient in 30 min; detector, UV 254 nm. This provided 6-((1R,5S,6r)-6-(((2,3-difluoropyridin-4-yl)oxy)methyl)-3-azabicyclo[3.1.0]hexan-3-yl)-1-(oxetan-3-yl)-1H-pyrazolo[3,4-b]pyrazine (48.8 mg, 46.0%) as a white solid. ¹H NMR (400 MHZ, DMSO-d₆) δ 8.18 (s, 1H), 8.07 (s, 1H), 7.96-7.85 (m, 1H), 7.26 (t, J=5.7 Hz, 1H), 5.91-5.82 (m, 1H), 5.09-5.02 (m, 2H), 5.01-4.91 (m, 2H), 4.21 (d, J=7.3 Hz, 2H), 3.96-3.87 (m, 2H), 3.65-3.52 (m, 2H), 1.97-1.89 (m, 2H), 1.29-1.14 (m, 1H). MS m/z: 401.2 [M+H]⁺.

### 6-((3S,5R)-3-Methyl-5-(((2-(trifluoromethyl)pyridin-3-yl)oxy)methyl)piperidin-1-yl)-1-(oxetan-3-ylmethyl)-1H-pyrazolo[3,4-b]pyrazine (229a) and 6-((3S,5S)-3-Methyl-5-(((2-(trifluoromethyl)pyridin-3-yl)oxy)methyl)piperidin-1-yl)-1-(oxetan-3-ylmethyl)-1H-pyrazolo[3,4-b]pyrazine(229b)

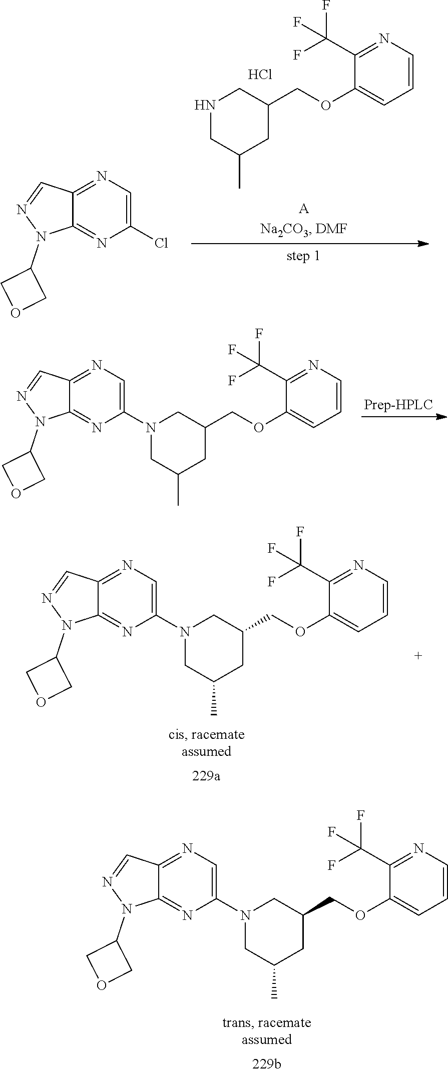

Followed General Procedure C using 3-((5-methylpiperidin-3-yl)methoxy)-2-(trifluoromethyl)pyridine hydrochloride (81.1 mg, 0.261 mmol, 1.1 equiv) and 6-chloro-1-(oxetan-3-yl)-1H-pyrazolo[3,4-b]pyrazine (50 mg, 0.237 mmol, 1.00 equiv) and Na₂CO₃ (75.5 mg, 0.711 mmol, 3.0 equiv) to afford 6-(3-methyl-5-(((2-(trifluoromethyl)pyridin-3-yl)oxy)methyl)piperidin-1-yl)-1-(oxetan-3-yl)-1H-pyrazolo[3,4-b]pyrazine (67 mg) as a white solid. This product was further purified by prep. HPLC with the following conditions: column: XBridge Prep F-Phenyl OBD Column, 19*100 mm, 5 um; Mobile Phase A: Water(0.1% FA), Mobile Phase B: ACN; Flow rate: 25 mL/min; Gradient: 45% B to 71% B in 7 min, 71% B; Wave Length: 254/220 nm; RT1(min): 6.35. This provided 6-((3S,5R)-3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine (229a, assumed structure, 36.4 mg, 54.3%) and 6-((3S,5S)-3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine (229b, assumed structure, 13.6 mg, 20.3%) as white solids.

229a: ¹H NMR (400 MHZ, DMSO-d₆) δ 8.37 (s, 1H), 8.26-8.20 (m, 1H), 8.14 (s, 1H), 7.73 (d, J=8.6 Hz, 1H), 7.65-7.58 (m, 1H), 5.86-5.74 (m, 1H), 5.04-4.95 (m, 2H), 4.95-4.88 (m, 1H), 4.88-4.80 (m, 1H), 4.19-4.09 (m, 2H), 4.05-3.96 (m, 1H), 3.95-3.86 (m, 1H), 3.86-3.78 (m, 1H), 3.31-3.25 (m, 1H), 2.35 (s, 1H), 2.02-1.95 (m, 1H), 1.85-1.75 (m, 1H), 1.60-1.49 (m, 1H), 0.94 (d, J=6.7 Hz, 3H). MS m/z: 449.2 [M+H]⁺.

229b: ¹H NMR (400 MHZ, DMSO-d₆) δ 8.44 (s, 1H), 8.32-8.26 (m, 1H), 8.19 (s, 1H), 7.84 (d, J=8.6 Hz, 1H), 7.76-7.68 (m, 1H), 5.94-5.82 (m, 1H), 5.08-4.99 (m, 2H), 4.99-4.89 (m, 2H), 4.89-4.81 (m, 1H), 4.56-4.47 (m, 1H), 4.27-4.18 (m, 1H), 4.11-4.02 (m, 1H), 2.75-2.64 (m, 1H), 2.61-2.53 (m, 1H), 2.16-2.04 (m, 1H), 1.90 (d, J=12.8 Hz, 1H), 1.75-1.66 (m, 1H), 1.15-1.02 (m, 1H), 0.97 (d, J=6.5 Hz, 3H). MS m/z: 449.2 [M+H]⁺.

### 1-(2-Methoxyethyl)-6-((3S,5R)-3-methyl-5-(((2-(trifluoromethyl)pyridin-3-yl)oxy)methyl)piperidin-1-yl)-1H-pyrazolo[3,4-b]pyrazine (230a) and 1-(2-Methoxyethyl)-6-((3S,5S)-3-methyl-5-(((2-(trifluoromethyl)pyridin-3-yl)oxy)methyl)piperidin-1-yl)-1H-pyrazolo[3,4-b]pyrazine (230b)

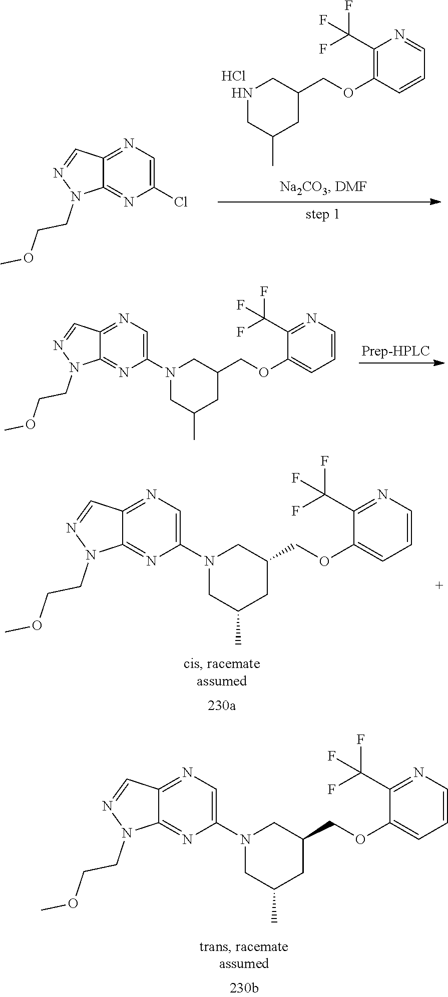 [SMILES: COCCn1ncc2ncc(Cl)nc21 ; Cl ; CC1CNCC(COc2cccnc2C(F)(F)F)C1 ; COCCn1ncc2ncc(N3CC(C)CC(COc4cccnc4C(F)(F)F)C3)nc21 ; COCCn1ncc2ncc(N3C[C@@H](C)C[C@@H](COc4cccnc4C(F)(F)F)C3)nc21 ; COCCn1ncc2ncc(N3C[C@@H](C)C[C@H](COc4cccnc4C(F)(F)F)C3)nc21]

Followed General Procedure C using 6-chloro-1-(2-methoxyethyl)-1H-pyrazolo[3,4-b]pyrazine (50 mg, 0.235 mmol, 1.00 equiv) and 3-[(5-methylpiperidin-3-yl)methoxy]-2-(trifluoromethyl)pyridine hydrochloride (70.95 mg, 0.259 mmol, 1.1 equiv) to afford 1-(2-methoxyethyl)-6-(3-methyl-5-(((2-(trifluoromethyl)pyridin-3-yl)oxy)methyl)piperidin-1-yl)-1H-pyrazolo[3,4-b]pyrazine (80 mg) as a yellow solid. This product was further purified by prep. HPLC with the following conditions: column: XBridge Prep F-Phenyl OBD Column, 19*100 mm, 5 um; Mobile Phase A: Water(0.1% FA), Mobile Phase B: ACN; Flow rate: 25 mL/min; Gradient: 45% B to 73% B in 7 min, 73% B; Wave Length: 254/220 nm; RT1(min): 6.37. This provided 1-(2-methoxyethyl)-6-((3S,5R)-3-methyl-5-(((2-(trifluoromethyl)pyridin-3-yl)oxy)methyl)piperidin-1-yl)-1H-pyrazolo[3,4-b]pyrazine (230a, 39.7 mg, 49.6%) and 1-(2-methoxyethyl)-6-((3S,5S)-3-methyl-5-(((2-(trifluoromethyl)pyridin-3-yl)oxy)methyl)piperidin-1-yl)-1H-pyrazolo[3,4-b]pyrazine (230b, 21.5 mg, 26.8%) as yellow solids.

230a: ¹H NMR (400 MHZ, DMSO-d₆) δ 8.35 (s, 1H), 8.23 (dd, J=4.5, 1.1 Hz, 1H), 7.99 (s, 1H), 7.77-7.71 (m, 1H), 7.66-7.59 (m, 1H), 4.34-4.25 (m, 2H), 4.16-4.10 (m, 2H), 3.97 (dd, J=13.2, 3.8 Hz, 1H), 3.89-3.83 (m, 2H), 3.71 (t, J=5.5 Hz, 2H), 3.28 (s, 1H), 3.16 (s, 3H), 2.40-2.30 (m, 1H), 2.04-1.92 (m, 1H), 1.84-1.74 (m, 1H), 1.60-1.48 (m, 1H), 0.95 (d, J=6.7 Hz, 3H). MS m/z:451.2 [M+H]⁺.

230b: ¹H NMR (400 MHZ, DMSO-d₆) δ 8.41 (s, 1H), 8.30-8.25 (m, 1H), 8.03 (s, 1H), 7.85-7.81 (m, 1H), 7.74-7.68 (m, 1H), 4.86 (d, J=13.4 Hz, 1H), 4.51 (d, J=12.8 Hz, 1H), 4.42-4.33 (m, 2H), 4.26-4.19 (m, 1H), 4.06 (dd, J=9.7, 7.9 Hz, 1H), 3.76 (t, J=5.5 Hz, 2H), 3.19 (s, 3H), 2.74-2.65 (m, 1H), 2.62-2.55 (m, 1H), 2.17-2.06 (m, 1H), 1.91 (d, J=12.7 Hz, 1H), 1.78-1.66 (m, 1H), 1.13-1.02 (m, 1H), 0.98 (d, J=6.6 Hz, 3H). MS m/z: 451.2 [M+H]⁺.

### (3-isopropyl-1H-pyrazol-5-yl)(2-methyl-5-(((2-(trifluoromethyl)pyridin-3-yl)oxy)methyl)piperidin-1-yl)methanone (231)

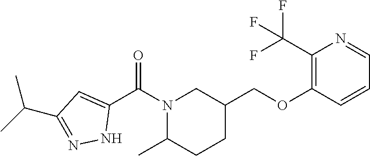 [SMILES: CC(C)c1cc(C(=O)N2CC(COc3cccnc3C(F)(F)F)CCC2C)[nH]n1]

Followed General Procedure E using 3-isopropyl-1H-pyrazole-5-carboxylic acid (50 mg, 0.324 mmol, 1 equiv) and 3-((6-methylpiperidin-3-yl)methoxy)-2-(trifluoromethyl)pyridine hydrochloride (111 mg, 0.356 mmol, 1.1 equiv) to afford (3-isopropyl-1H-pyrazol-5-yl)(2-methyl-5-(((2-(trifluoromethyl)pyridin-3-yl)oxy)methyl)piperidin-1-yl)methanone (56 mg, 41.9%) as a white solid. ¹H NMR (400 MHz, DMSO-d₆) δ 13.08-12.65 (m, 1H), 8.25 (s, 1H), 7.85-7.66 (m, 2H), 6.33-6.13 (m, 1H), 5.01-4.80 (m, 1H), 4.66-4.41 (m, 1H), 4.29-3.91 (m, 2H), 3.18-2.67 (m, 2H), 2.02-1.84 (m, 1H), 1.74-1.57 (m, 4H), 1.29-1.14 (m, 9H). MS m/z: 411.2 [M+H]⁺.

### 2-[2-Methyl-5-({[2-(trifluoromethyl)pyridin-3-yl]oxy}methyl)piperidin-1-yl]-6-(1,3,4-thiadiazol-2-yl)pyrazine (232)

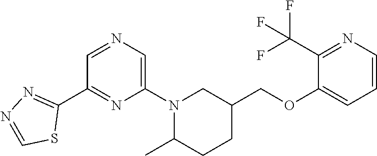 [SMILES: CC1CCC(COc2cccnc2C(F)(F)F)CN1c1cncc(-c2nncs2)n1]

Followed General Procedure C using 2-(6-chloropyrazin-2-yl)-1,3,4-thiadiazole (50 mg, 0.252 mmol, 1 equiv) and 3-((6-methylpiperidin-3-yl)methoxy)-2-(trifluoromethyl)pyridine hydrochloride (86 mg, 0.277 mmol, 1.1 equiv) to afford 2-(6-(2-methyl-5-(((2-(trifluoromethyl)pyridin-3-yl)oxy)methyl)piperidin-1-yl)pyrazin-2-yl)-1,3,4-thiadiazole (37.1 mg, 32.7%) as a light yellow solid. ¹H NMR (400 MHZ, DMSO-d₆) δ 9.75 (s, 1H), 8.61 (s, 1H), 8.46 (s, 1H), 8.28 (d, J=4.4 Hz, 1H), 7.87-7.83 (m, 1H), 7.75-7.70 (m, 1H), 4.80-4.72 (m, 1H), 4.52-4.45 (m, 1H), 4.28-4.22 (m, 1H), 4.21-4.14 (m, 1H), 2.97-2.88 (m, 1H), 2.12-2.01 (m, 1H), 1.86-1.62 (m, 4H), 1.22-1.18 (m, 3H). MS m/z: 437.1 [M+H]⁺.

### 6-((3S,5R)-3-Methyl-5-(((2-(trifluoromethyl)pyridin-3-yl)oxy)methyl)piperidin-1-yl)-1-(oxetan-3-ylmethyl)-1H-pyrazolo[3,4-b]pyrazine (233a) and 6-((3S,5S)-3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine (233b)

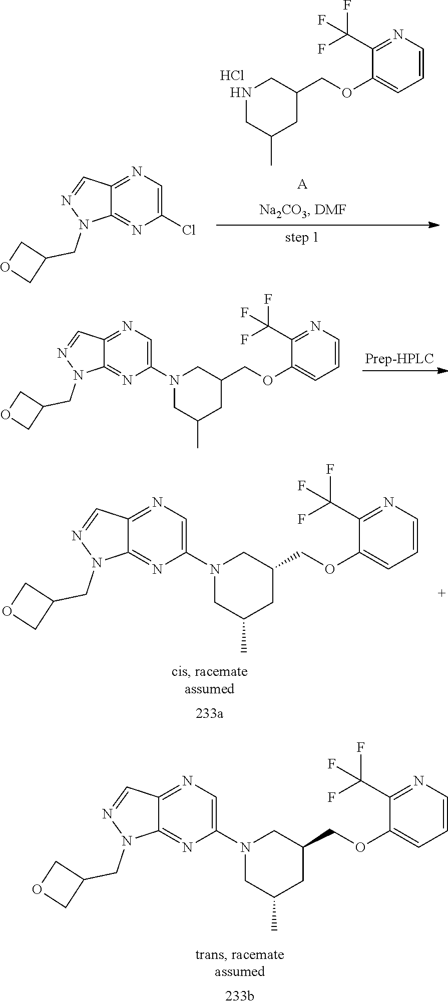

Followed General Procedure C using 6-chloro-1-(oxetan-3-ylmethyl)-1H-pyrazolo[3,4-b]pyrazine (50 mg, 0.223 mmol, 1 equiv) and 3-((5-methylpiperidin-3-yl)methoxy)-2-(trifluoromethyl)pyridine hydrochloride to afford 6-(3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine as a white solid. This product was further purified by prep. HPLC with the following conditions: Column: XBridge Prep F-Phenyl OBD Column, 19*100 mm, 5 um; Mobile Phase A: Water (0.1% FA), Mobile Phase B: ACN; Flow rate: 25 mL/min; Gradient: 45% B to 71% B in 7 min, 71% B; Wavelength: 254/220 nm; RT1(min): 6.35. This provided 6-((3S,5R)-3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine (233a, assumed structure, 41.0 mg, 39.3%) and 6-((3S,5S)-3-methyl-5-(((2-(trifluoromethyl)pyridin-3-yl)oxy)methyl)piperidin-1-yl)-1-(oxetan-3-ylmethyl)-1H-pyrazolo[3,4-b]pyrazine (233b, assumed structure, 18.0 mg, 17.4%) as white solids.

233a: ¹H NMR (300 MHz, Methanol-d₄) δ 8.36 (s, 1H), 8.29-8.25 (m, 1H), 7.96 (s, 1H), 7.74-7.68 (m, 1H), 7.66-7.60 (m, 1H), 4.88-4.81 (m, 2H), 4.72-4.65 (m, 2H), 4.61-4.55 (m, 2H), 4.28-4.09 (m, 4H), 4.00-3.92 (m, 1H), 3.64-3.50 (m, 1H), 3.44 (s, 1H), 2.56 (s, 1H), 2.19-1.95 (m, 2H), 1.81-1.69 (m, 1H), 1.18-1.09 (m, 3H). MS m/z: 463.1 [M+H]⁺.

233b: ¹H NMR (400 MHZ, Methanol-d₄) δ 8.32 (s, 1H), 8.22 (dd, J=4.6, 1.2 Hz, 1H), 7.92 (s, 1H), 7.76-7.71 (m, 1H), 7.63 (dd, J=8.6, 4.6 Hz, 1H), 5.04-4.97 (m, 1H), 4.83-4.75 (m, 2H), 4.67-4.59 (m, 4H), 4.58-4.52 (m, 1H), 4.23 (dd, J=9.4, 4.6 Hz, 1H), 4.05-3.99 (m, 1H), 3.60-3.49 (m, 1H), 2.81-2.71 (m, 1H), 2.69-2.59 (m, 1H), 2.29-2.17 (m, 1H), 2.04-1.96 (m, 1H), 1.87-1.75 (m, 1H), 1.22-1.11 (m, 1H), 1.08-1.03 (m, 3H). MS m/z: 463.1 [M+H]⁺.

### (3-Isopropyl-1H-pyrazol-5-yl)((3S,5R)-3-methyl-5-(((2-(trifluoromethyl)pyridin-3-yl)oxy)methyl)piperidin-1-yl)methanone (234a) and (3-Isopropyl-1H-pyrazol-5-yl)((3S,5S)-3-methyl-5-(((2-(trifluoromethyl)pyridin-3-yl)oxy)methyl)piperidin-1-yl)methanone (234b)

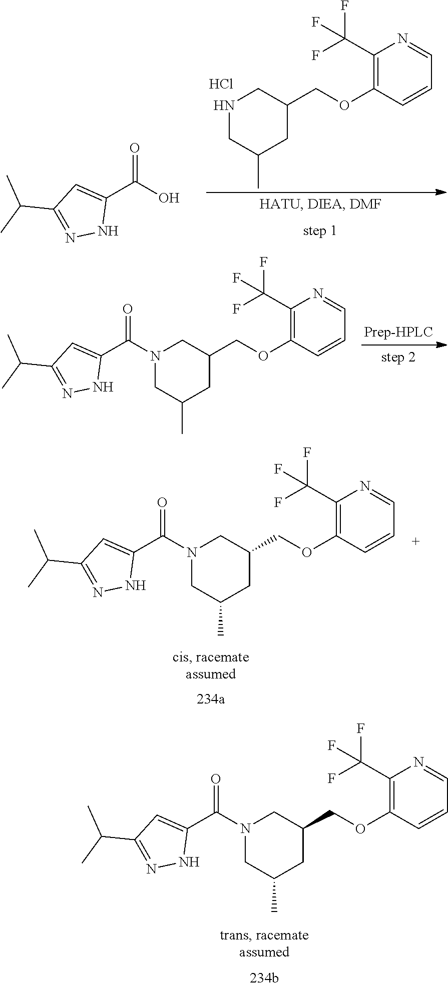 [SMILES: CC(C)c1cc(C(=O)O)[nH]n1 ; Cl ; CC1CNCC(COc2cccnc2C(F)(F)F)C1 ; CC1CC(COc2cccnc2C(F)(F)F)CN(C(=O)c2cc(C(C)C)n[nH]2)C1 ; CC(C)c1cc(C(=O)N2C[C@@H](C)C[C@@H](COc3cccnc3C(F)(F)F)C2)[nH]n1 ; CC(C)c1cc(C(=O)N2C[C@@H](C)C[C@H](COc3cccnc3C(F)(F)F)C2)[nH]n1]

Followed General Procedure E using 3-isopropyl-1H-pyrazole-5-carboxylic acid (50 mg, 0.324 mmol, 1 equiv) and 3-((5-methylpiperidin-3-yl)methoxy)-2-(trifluoromethyl)pyridine hydrochloride (111 mg, 0.356 mmol, 1.1 equiv) to afford (3-isopropyl-1H-pyrazol-5-yl)((3S,5R)-3-methyl-5-(((2-(trifluoromethyl)pyridin-3-yl)oxy)methyl)piperidin-1-yl)methanone (100 mg) as a light yellow solid. This product was further purified by prep. HPLC with the following conditions: Column: XBridge Prep F-Phenyl OBD Column, 19*100 mm, 5 um; Mobile Phase A: Water(0.1% FA), Mobile Phase B: ACN; Flow rate: 25 mL/min; Gradient: 35% B to 56% B in 8 min, 56% B; Wave Length: 254/220 nm; RT1(min): 7.22. This provided (3-isopropyl-1H-pyrazol-5-yl)((3S,5R)-3-methyl-5-(((2-(trifluoromethyl)pyridin-3-yl)oxy)methyl)piperidin-1-yl)methanone (234a, 58.4 mg, 43.3%) and (3-isopropyl-1H-pyrazol-5-yl)((3S,5S)-3-methyl-5-(((2-(trifluoromethyl)pyridin-3-yl)oxy)methyl)piperidin-1-yl)methanone (234b, 32.9 mg, 24.3%) as light yellow solids.

234a: ¹H NMR (400 MHZ, CD₃OD) δ 8.24-8.11 (m, 1H), 7.75-7.55 (m, 2H), 6.40-6.03 (m, 1H), 4.24-3.93 (m, 4H), 3.84-3.62 (m, 1H), 3.18-2.96 (m, 1H), 2.95-2.81 (m, 1H), 2.47-2.27 (m, 1H), 2.03-1.83 (m, 2H), 1.66-1.56 (m, 1H), 1.29 (d, J=6.9 Hz, 2H), 1.16 (dd, J=21.2, 7.0 Hz, 4H), 1.02 (d, J=6.6 Hz, 2H), 0.89 (d, J=6.4 Hz, 1H). MS m/z: 411.2 [M+H]⁺.

234b: ¹H NMR (400 MHZ, CD₃OD) δ 8.26-8.13 (m, 1H), 7.79-7.52 (m, 2H), 6.42-6.19 (m, 1H), 4.69-4.41 (m, 2H), 4.17-3.80 (m, 2H), 3.13-3.00 (m, 1H), 2.96-2.28 (m, 2H), 2.26-2.09 (m, 1H), 2.09-1.92 (m, 1H), 1.86-1.71 (m, 1H), 1.30 (d, J=6.8 Hz, 6H), 1.26-0.88 (m, 4H). MS m/z: 411.1 [M+H]⁺.

### (3-((2-Methoxyphenoxy)methyl)piperidin-1-yl)(5-methyl-6-phenyl-5H-pyrrolo[2,3-b]pyrazin-7-yl)methanone (235)

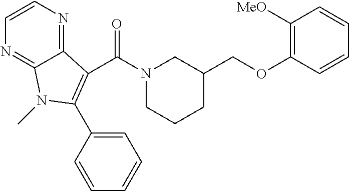 [SMILES: COc1ccccc1OCC1CCCN(C(=O)c2c(-c3ccccc3)n(C)c3nccnc23)C1]

Step 1: tert-butyl 3-((2-methoxyphenoxy)methyl)piperidine-1-carboxylate: Followed General Procedure A using tert-butyl 3-(hydroxymethyl)piperidine-1-carboxylate (200 mg, 0.929 mmol, 1 equiv) and 2-methoxyphenol (173 mg, 1.39 mmol, 1.5 equiv) to afford tert-butyl 3-(2-methoxyphenoxymethyl)piperidine-1-carboxylate (180 mg, 50.0%) as a yellow solid MS m/z: 322 [M+H]⁺.

Step 2: 3-((2-methoxyphenoxy)methyl)piperidine hydrochloride: Followed General Procedure B using tert-butyl 3-((2-methoxyphenoxy)methyl)piperidine-1-carboxylate (180 mg, 1.38 mmol, 1.00 equiv) to afford the crude product 3-(piperidin-3-ylmethoxy)-2-(trifluoromethyl)pyridine hydrochloride (120 mg). MS m/z: 222 [M+H]⁺.

Step 3: (3-((2-methoxyphenoxy)methyl)piperidin-1-yl)(5-methyl-6-phenyl-5H-pyrrolo[2,3-b]pyrazin-7-yl)methanone: Followed General Procedure E using 5-methyl-6-phenylpyrrolo[2,3-b]pyrazine-7-carboxylic acid (30 mg, 0.118 mmol, 1 equiv) and 3-((2-methoxyphenoxy)methyl)piperidine hydrochloride (26.2 mg, 0.118 mmol, 1 equiv). The resulting mixture was purified by reverse flash chromatography with the following conditions: column, C18 silica gel; mobile phase, MeCN in Water (0.1% FA), 10% to 100% gradient in 20 min; detector, UV 254 nm. This provided 3-(2-methoxyphenoxymethyl)-1-{5-methyl-6-phenylpyrrolo[2,3-b]pyrazine-7-carbonyl}piperidine (24 mg, 43.5%) as a white solid. ¹H NMR (300 MHz, DMSO-d₆) δ 8.54-8.21 (m, 2H), 7.68-7.42 (m, 5H), 7.01-6.68 (m, 4H), 4.51-4.19 (m, 1H), 3.88-3.59 (m, 6H), 3.00-2.75 (m, 2H), 1.88-1.63 (m, 2H), 1.54-1.02 (m, 3H). MS m/z: 405.15 [M+H]⁺.

### (5-Methyl-6-phenyl-5H-pyrrolo[2,3-b]pyrazin-7-yl)(3-((2-(trifluoromethoxy)phenoxy)methyl)piperidin-1-yl)methanone (236)

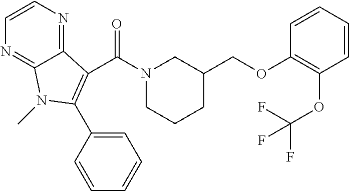 [SMILES: Cn1c(-c2ccccc2)c(C(=O)N2CCCC(COc3ccccc3OC(F)(F)F)C2)c2nccnc21]

Step 1: tert-butyl 3-((2-(trifluoromethoxy)phenoxy)methyl)piperidine-1-carboxylate: Followed General Procedure A using tert-butyl 3-(hydroxymethyl)piperidine-1-carboxylate (200 mg, 0.929 mmol, 1 equiv) and 2-(trifluoromethoxy)phenol (173 mg, 1.39 mmol, 1.5 equiv) to afford tert-butyl 3-(2-methoxyphenoxymethyl)piperidine-1-carboxylate (180 mg, 50%) as a yellow solid MS m/z: 322 [M+H]⁺.

Step 2: 3-((2-(trifluoromethoxy)phenoxy)methyl)piperidine hydrochloride: Followed General Procedure B using tert-butyl 3-((2-(trifluoromethoxy)phenoxy)methyl)piperidine-1-carboxylate (180 mg, 1.38 mmol, 1.00 equiv) to afford the crude product 3-((2-(trifluoromethoxy)phenoxy)methyl)piperidine hydrochloride (120 mg). MS m/z: 276 [M+H]⁺.

Step 3: (5-methyl-6-phenyl-5H-pyrrolo[2,3-b]pyrazin-7-yl)(3-((2-(trifluoromethoxy)phenoxy)methyl)piperidin-1-yl)methanone: Followed General Procedure E using 5-methyl-6-phenylpyrrolo[2,3-b]pyrazine-7-carboxylic acid (30 mg, 0.118 mmol, 1 equiv) and 3-((2-(trifluoromethoxy)phenoxy)methyl)piperidine hydrochloride (32.6 mg, 0.118 mmol, 1 equiv). The crude product was purified by reverse flash chromatography with the following conditions: column, C18 silica gel; mobile phase, MeCN in Water (0.1% FA), 10% to 100% gradient in 20 min; detector, UV 254 nm. This provided 1-{5-methyl-6-phenylpyrrolo[2,3-b]pyrazine-7-carbonyl}-3-[2-(trifluoromethoxy)phenoxymethyl]piperidine (29.1 mg, 47.7%) as a white solid. ¹H NMR (300 MHZ, DMSO-d₆) δ 8.55-8.24 (m, 2H), 7.67-7.42 (m, 5H), 7.40-6.90 (m, 4H), 4.53-4.26 (m, 1H), 4.01-3.93 (m, 1H), 3.81-3.68 (m, 4H), 3.68-3.61 (m, 1H), 3.08-2.72 (m, 2H), 1.95-1.55 (m, 2H), 1.45-1.19 (m, 2H), 0.99 (d, J=6.5 Hz, 1H). MS m/z: 511.15 [M+H]⁺.

### 4-(2-(6-(3-(((2-(trifluoromethyl)pyridin-3-yl)oxy)methyl)piperidin-1-yl)-1H-pyrazolo[3,4-b]pyrazin-1-yl)ethyl)morpholine (237)

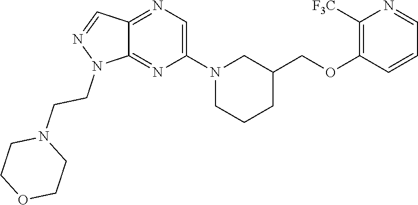 [SMILES: FC(F)(F)c1ncccc1OCC1CCCN(c2cnc3cnn(CCN4CCOCC4)c3n2)C1]

Followed General Procedure C using 4-(2-(6-chloro-1H-pyrazolo[3,4-b]pyrazin-1-yl)ethyl)morpholine (60 mg, 0.224 mmol, 1 equiv) and 3-(piperidin-3-ylmethoxy)-2-(trifluoromethyl)pyridine hydrochloride (64.1 mg, 0.246 mmol, 1.1 equiv). The crude product was purified by reverse flash chromatography with the following conditions (column, C18 gel; mobile phase, B phase: MeCN, A phase: water; 10% to 90% B gradient in 10 min; detector: UV 254/220 nm). This provided 4-(2-(6-(3-(((2-(trifluoromethyl)pyridin-3-yl)oxy)methyl)piperidin-1-yl)-1H-pyrazolo[3,4-b]pyrazin-1-yl)ethyl)morpholine (30 mg, 27.1%) as a yellow solid. ¹H NMR (400 MHZ, DMSO-d₆) δ 8.31 (s, 1H), 8.24 (d, 1H), 7.99 (s, 1H), 7.76 (d, J=8.6 Hz, 1H), 7.69-7.62 (m, 1H), 4.58 (d, J=13.2, 3.8 Hz, 1H), 4.30 (t, 3H), 4.21-4.13 (m, 1H), 4.03 (t, J=9.7, 8.0 Hz, 1H), 3.67 (s, 2H), 3.44-3.34 (m, 4H), 3.14-3.04 (m, 1H), 2.99-2.90 (m, 1H), 2.70 (t, J=6.3 Hz, 2H), 2.40-2.29 (m, 4H), 2.13-2.00 (m, 1H), 1.92-1.83 (m, 1H), 1.82-1.72 (m, 1H), 1.44 (s, 2H). MS m/z: 492.1 [M+H]⁺.

### N,N-Dimethyl-2-(6-(3-(((2-(trifluoromethyl)pyridin-3-yl)oxy)methyl)piperidin-1-yl)-1H-pyrazolo[3,4-b]pyrazin-1-yl)ethan-1-amine (238)

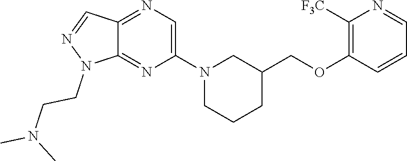 [SMILES: CN(C)CCn1ncc2ncc(N3CCCC(COc4cccnc4C(F)(F)F)C3)nc21]

Followed General Procedure C using 3-(piperidin-3-ylmethoxy)-2-(trifluoromethyl)pyridine hydrochloride (60 mg, 0.231 mmol, 1 equiv) and 2-(6-chloro-1H-pyrazolo[3,4-b]pyrazin-1-yl)-N,N-dimethylethan-1-amine (62.43 mg, 0.277 mmol, 1.2 equiv). The crude product was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford N,N-dimethyl-2-(6-(3-(((2-(trifluoromethyl)pyridin-3-yl)oxy)methyl)piperidin-1-yl)-1H-pyrazolo[3,4-b]pyrazin-1-yl)ethan-1-amine (36 mg, 32.83%) as a yellow green solid. ¹H NMR (400 MHZ, DMSO-d₆) δ 8.37 (s, 1H), 8.27 (s, 1H), 8.01 (s, 1H), 7.82 (d, J=8.4 Hz, 1H), 7.72-7.67 (m, 1H), 4.65-4.57 (m, 1H), 4.32 (d, J=19.0 Hz, 3H), 4.20 (s, 1H), 4.09 (t, J=8.8 Hz, 1H), 3.14 (s, 1H), 3.03 (d, J=11.7 Hz, 1H), 2.72-2.66 (m, 2H), 2.11 (s, 7H), 1.91 (s, 1H), 1.81 (s, 1H), 1.58 (s. 1H), 1.48 (s, 1H). MS m/z:450.25 [M+H]⁺.

### N-((1-(1-(2,2-Difluoroethyl)-1H-pyrazolo[3,4-b]pyrazin-6-yl)piperidin-3-yl)methyl)-N-methyl-6-(trifluoromethyl)pyridin-2-amine (239)

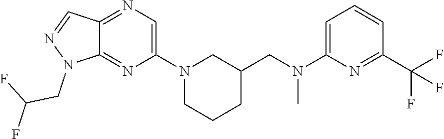 [SMILES: CN(CC1CCCN(c2cnc3cnn(CC(F)F)c3n2)C1)c1cccc(C(F)(F)F)n1]

Step 1: tert-butyl 3-((methyl(6-(trifluoromethyl)pyridin-2-yl)amino)methyl)piperidine-1-carboxylate: Followed General Procedure D using tert-butyl 3-((methylamino)methyl)piperidine-1-carboxylate (448 mg, 2.89 mmol, 1.1 equiv) and 2-fluoro-6-(trifluoromethyl)pyridine (600 mg, 2.628 mmol, 1 equiv) to afford tert-butyl 3-((methyl(6-(trifluoromethyl)pyridin-2-yl)amino)methyl)piperidine-1-carboxylate (250 mg, 25.22%) as a white solid. MS m/z: 374 [M+H]⁺.

Step 2: N-methyl-N-(piperidin-3-ylmethyl)-6-(trifluoromethyl)pyridin-2-amine hydrochloride: Followed General Procedure B using tert-butyl 3-((methyl(6-(trifluoromethyl)pyridin-2-yl)amino)methyl)piperidine-1-carboxylate (250 mg, 0.669 mmol, 1 equiv) to afford the crude product N-methyl-N-(piperidin-3-ylmethyl)-6-(trifluoromethyl)pyridin-2-amine hydrochloride(200 mg). MS m/z: 274 [M+H]⁺.

Step 3: N-((1-(1-(2,2-difluoroethyl)-1H-pyrazolo[3,4-b]pyrazin-6-yl)piperidin-3-yl)methyl)-N-methyl-6-(trifluoromethyl)pyridin-2-amine: Followed General Procedure C using 6-chloro-1-(2,2-difluoroethyl)-1H-pyrazolo[3,4-b]pyrazine (60 mg, 0.274 mmol, 1 equiv) and N-methyl-N-(piperidin-3-ylmethyl)-6-(trifluoromethyl)pyridin-2-amine hydrochloride (90.0 mg, 0.329 mmol, 1.2 equiv). The crude was purified by reverse flash chromatography with the following conditions (column, C18 gel; mobile phase, B phase: MeCN, A phase: water; 0% to 100% B gradient in 20 min; detector: UV 254/220 nm). This provided N-((1-(1-(2,2-difluoroethyl)-1H-pyrazolo[3,4-b]pyrazin-6-yl)piperidin-3-yl)methyl)-N-methyl-6-(trifluoromethyl)pyridin-2-amine (41.5 mg, 33.16%) as a white solid. ¹H NMR (400 MHZ, DMSO-d₆) δ 8.40 (s, 1H), 8.10 (s, 1H), 7.69 (t, J=8.0 Hz, 1H), 6.93 (d, J=7.7 Hz, 2H), 6.55-6.22 (m, 1H), 4.65-4.53 (m, 2H), 4.31 (d, J=13.3 Hz, 2H), 3.69-3.60 (m, 1H), 3.52-3.44 (m, 1H), 3.21-3.12 (m, 1H), 3.08 (s, 3H), 2.98-2.89 (m, 1H), 2.07-1.96 (m, 1H), 1.78 (d, J=10.5 Hz, 2H), 1.54-1.30 (m, 2H). MS m/z: 456.2 [M+H]⁺.

### 1-(4-((1-(1-(2,2-difluoroethyl)-1H-pyrazolo[3,4-b]pyrazin-6-yl)piperidin-3-yl)methoxy)piperidin-1-yl)ethan-1-one (240)

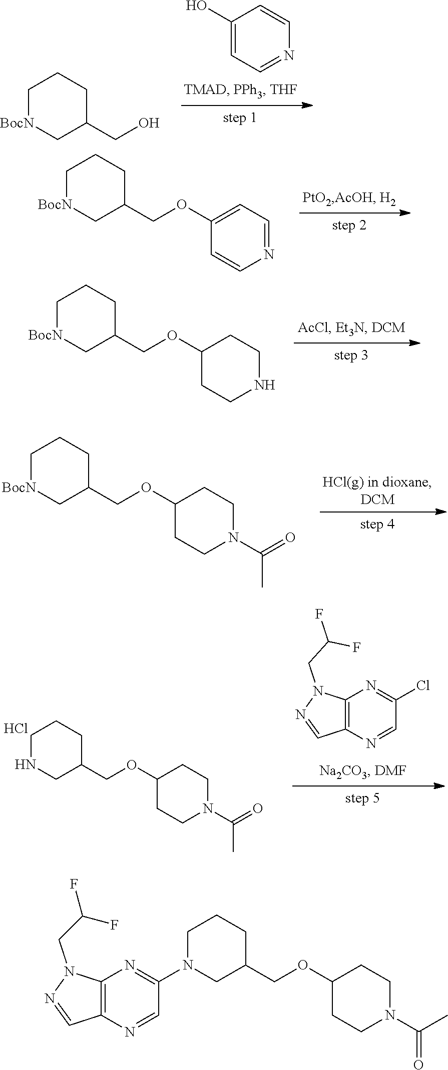

Step 1: tert-butyl 3-((pyridin-4-yloxy)methyl)piperidine-1-carboxylate: A solution of tert-butyl 3-(hydroxymethyl)piperidine-1-carboxylate (200 mg, 0.929 mmol, 1 equiv), pyridin-4-ol (97.2 mg, 1.02 mmol, 1.1 equiv), TMAD (256 mg, 1.48 mmol, 1.6 equiv) and PPh₃ (390 mg, 1.49 mmol, 1.6 equiv) in THF (3 mL) was stirred for overnight at room temperature under nitrogen atmosphere. The resulting mixture was concentrated under vacuum. The residue was purified by silica gel column chromatography, eluted with PE/EA (1:1) to afford tert-butyl 3-((pyridin-4-yloxy)methyl)piperidine-1-carboxylate (150 mg, 55.2%). MS m/z: 293[M+H]⁺.

Step 2. tert-butyl 3-((piperidin-4-yloxy)methyl)piperidine-1-carboxylate: A solution of tert-butyl 3-((pyridin-4-yloxy)methyl)piperidine-1-carboxylate (150 mg, 0.513 mmol, 1 equiv) and PtO₂ (23.3 mg, 0.103 mmol, 0.2 equiv) in AcOH (2 mL) was stirred for overnight at 40° C. under hydrogen atmosphere. The resulting mixture was concentrated under vacuum. The crude product (65 mg) was used in the next step directly without further purification. MS m/z: 299 [M+H]⁺.

Step 3. tert-butyl 3-(((1-acetylpiperidin-4-yl)oxy)methyl)piperidine-1-carboxylate: A solution of tert-butyl 3-((piperidin-4-yloxy)methyl)piperidine-1-carboxylate (65 mg, 0.216 mmol, 1 equiv), AcCl (25.5 mg, 0.324 mmol, 1.5 equiv) and Et₃N (65.7 mg, 0.648 mmol, 3 equiv) in DCM (3 mL) was stirred for 3 h at room temperature. The resulting mixture was concentrated under vacuum. The residue was purified by reverse flash chromatography with the following conditions: column, C18 silica gel; mobile phase, MeCN in water, 10% to 100% gradient in 30 min; detector, UV 254 nm. This provided tert-butyl 3-(((1-acetylpiperidin-4-yl)oxy)methyl)piperidine-1-carboxylate (37 mg, 33.6%) as a white solid. MS m/z: 341 [M+H]⁺.

Step 4. 1-(4-(piperidin-3-ylmethoxy)piperidin-1-yl)ethan-1-one hydrochloride: Followed General Procedure B using tert-butyl 3-(((1-acetylpiperidin-4-yl)oxy)methyl)piperidine-1-carboxylate (37 mg, 0.109 mmol, 1 equiv) to afford the crude product 1-(4-(piperidin-3-ylmethoxy)piperidin-1-yl)ethan-1-one hydrochloride (30 mg). MS m/z: 241 [M+H]⁺.

Step 5. 1-(4-((1-(1-(2,2-difluoroethyl)-1H-pyrazolo[3,4-b]pyrazin-6-yl)piperidin-3-yl)methoxy)piperidin-1-yl)ethan-1-one: Followed General Procedure C using 1-(4-(piperidin-3-ylmethoxy)piperidin-1-yl)ethan-1-one hydrochloride (30 mg, 0.125 mmol, 1 equiv) and Na₂CO₃ (26.5 mg, 0.250 mmol, 2 equiv) in DMF (1 mL) was stirred for 2 h at 100° C. under. The resulting mixture was purified by reverse flash chromatography with the following conditions: column, C18 silica gel; mobile phase, MeCN in water, 10% to 100% gradient in 30 min; detector, UV 254 nm. This provided 1-(4-((1-(1-(2,2-difluoroethyl)-1H-pyrazolo[3,4-b]pyrazin-6-yl)piperidin-3-yl)methoxy)piperidin-1-yl)ethan-1-one (22 mg, 41.4%) as an off-white solid. ¹H NMR (400 MHZ, DMSO-d₆) δ 8.42 (s, 1H), 8.11 (s, 1H), 6.43 (tt, J=55.0, 3.9 Hz, 1H), 4.66 (td, J=14.9, 3.9 Hz, 2H), 4.40 (d, J=13.2 Hz, 1H), 4.28 (d, J=13.3 Hz, 1H), 3.82-3.68 (m, 1H), 3.66-3.54 (m, 1H), 3.53-3.44 (m, 1H), 3.40-4.33 (m, 2H), 3.26-3.11 (m, 3H), 3.03-2.95 (m, 1H), 1.98 (d, J=1.8 Hz, 3H), 1.89-1.64 (m, 5H), 1.59-1.43 (m, 2H), 1.42-1.27 (m, 2H). MS m/z: 422.9 [M+H]⁺.

### (2-(4-Fluorophenyl)-1-methyl-1H-imidazo[4,5-b]pyrazin-5-yl)((1R,5S,6r)-6-(((6-(trifluoromethyl)pyridin-2-yl)oxy)methyl)-3-azabicyclo[3.1.0]hexan-3-yl)methanone (241)

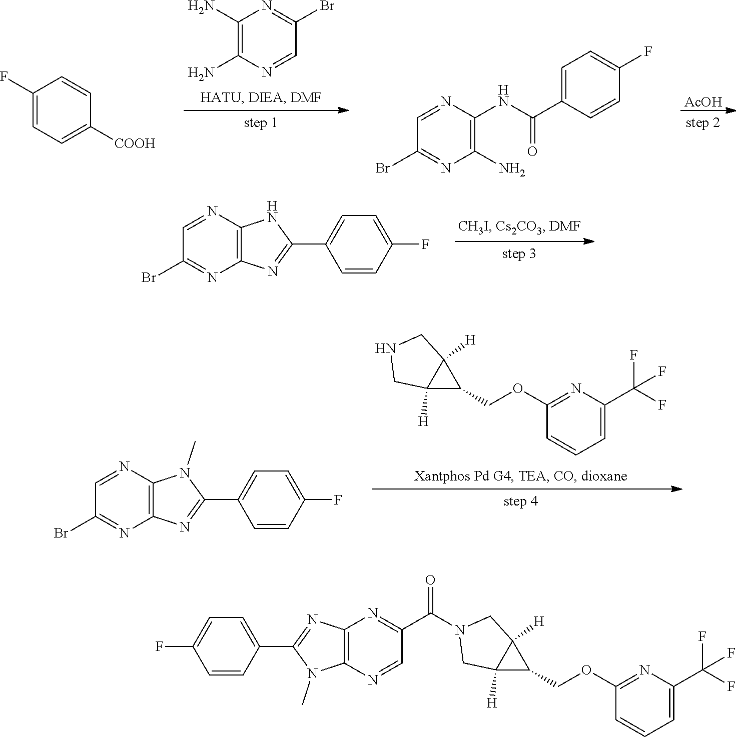

Step 1: N-(3-amino-5-bromopyrazin-2-yl)-4-fluorobenzamide: A solution of 4-fluorobenzoic acid (1 g, 7.14 mmol, 1 equiv), 5-bromopyrazine-2,3-diamine (2.16 g, 11.4 mmol, 1.6 equiv), HATU (5.43 g, 14.3 mmol, 2 equiv) and DIPEA (2.77 g, 21.4 mmol, 3 equiv) in DMF (10 mL) was stirred for overnight at 70° C. The resulting mixture was diluted with EtOAc (50 mL). The organic layer was washed with water (3×20 mL), dried over anhydrous Na₂SO₄. After filtration, the filtrate was concentrated under reduced pressure. The residue was purified by silica gel column chromatography, eluted with PE/EA (1:1) to afford N-(3-amino-5-bromopyrazin-2-yl)-4-fluorobenzamide (800 mg, 36.0%). MS m/z: 311[M+H]⁺.

Step 2: 5-bromo-2-(4-fluorophenyl)-1H-imidazo[4,5-b]pyrazine: A solution of N-(3-amino-5-bromopyrazin-2-yl)-4-fluorobenzamide (800 mg, 2.57 mmol, 1 equiv) in AcOH (10 mL) was stirred for overnight at 100° C. The resulting mixture was concentrated under vacuum. The resulting mixture was diluted with CH₂Cl₂ (50 mL). The combined organic layers were washed with NaHCO₃aq. (2×30 mL), dried over anhydrous Na₂SO₄. After filtration, the filtrate was concentrated under reduced pressure. The residue was purified by silica gel column chromatography, eluted with PE/EA (1:1) to afford 5-bromo-2-(4-fluorophenyl)-1H-imidazo[4,5-b]pyrazine (600 mg, 79.6%). MS m/z: 293 [M+H]⁺.

Step 3: 5-bromo-2-(4-fluorophenyl)-1-methyl-1H-imidazo[4,5-b]pyrazine: A solution of 5-bromo-2-(4-fluorophenyl)-1H-imidazo[4,5-b]pyrazine (600 mg, 2.04 mmol, 1 equiv), MeI (319 mg, 2.252 mmol, 1.1 equiv) and Cs₂CO₃ (1.3 g, 4.09 mmol, 2 equiv) in DMF (4 mL) was stirred for overnight at room temperature. The residue was purified by reverse flash chromatography with the following conditions: column, C18 silica gel; mobile phase, MeCN in water, 10% to 100% gradient in 30 min; detector, UV 254 nm. This provided 5-bromo-2-(4-fluorophenyl)-1-methyl-1H-imidazo[4,5-b]pyrazine (300 mg, 47.7%). MS m/z: 307 [M+H]⁺.

Step 4. (2-(4-fluorophenyl)-1-methyl-1H-imidazo[4,5-b]pyrazin-5-yl)((1R,5S,6r)-6-(((6-(trifluoromethyl)pyridin-2-yl)oxy)methyl)-3-azabicyclo[3.1.0]hexan-3-yl)methanone: A solution of 5-bromo-2-(4-fluorophenyl)-1-methyl-1H-imidazo[4,5-b]pyrazine (60 mg, 0.195 mmol, 1 equiv), (1R,5S,6S)-6-({[6-(trifluoromethyl)pyridin-2-yl]oxy}methyl)-3-azabicyclo[3.1.0]hexane (55.5 mg, 0.215 mmol, 1.1 equiv), Et₃N (59.3 mg, 0.585 mmol, 3 equiv) and Xantphos Pd G4 (18.80 mg, 0.020 mmol, 0.1 equiv) in dioxane (2 mL) was stirred for overnight at 50° C. under carbon monoxide atmosphere. The residue was purified by silica gel column chromatography, eluted with PE/EA (1:1) to afford impure product. This was purified by reverse flash chromatography with the following conditions: column, C18 silica gel; mobile phase, MeCN in water, 10% to 100% gradient in 30 min; detector, UV 254 nm. This provided (2-(4-fluorophenyl)-1-methyl-1H-imidazo[4,5-b]pyrazin-5-yl)((1R,5S,6r)-6-(((6-(trifluoromethyl)pyridin-2-yl)oxy)methyl)-3-azabicyclo[3.1.0]hexan-3-yl)methanone (40 mg, 39.2%) as a white solid. NMR (300 MHZ, DMSO-d₆) δ 8.76 (s, 1H), 8.15-8.05 (m, 2H), 7.96 (t, J=7.9 Hz, 1H), 7.57-7.43 (m, 3H), 7.16 (d, J=8.5 Hz, 1H), 4.23 (dd, J=7.3, 2.5 Hz, 2H), 4.04-3.94 (m, 5H), 3.88 (dd, J=11.3, 3.0 Hz, 1H), 3.63-3.56 (m, 1H), 1.84-1.77 (m, 2H), 1.11 (dt, J=7.1, 3.3 Hz, 1H). MS m/z: 512.8 [M+H]⁺.

### (2-(4-Fluorophenyl)-1-methyl-1H-imidazo[4,5-b]pyrazin-6-yl)((1R,5S,6r)-6-(((6-(trifluoromethyl)pyridin-2-yl)oxy)methyl)-3-azabicyclo[3.1.0]hexan-3-yl)methanone (242)

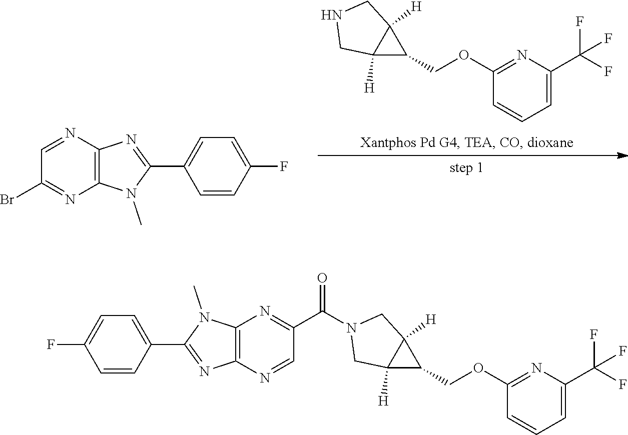 [SMILES: FC(F)(F)c1cccc(OC[C@H]2[C@@H]3CNC[C@@H]32)n1 ; Cn1c(-c2ccc(F)cc2)nc2ncc(Br)nc21 ; Cn1c(-c2ccc(F)cc2)nc2ncc(C(=O)N3C[C@@H]4[C@@H](COc5cccc(C(F)(F)F)n5)[C@@H]4C3)nc21]

A solution of 6-bromo-2-(4-fluorophenyl)-1-methylimidazo[4,5-b]pyrazine (30 mg, 0.098 mmol, 1 equiv), (1R,5S,6S)-6-({[6-(trifluoromethyl)pyridin-2-yl]oxy}methyl)-3-azabicyclo[3.1.0]hexane (27.8 mg, 0.108 mmol, 1.1 equiv), Et₃N (29.7 mg, 0.294 mmol, 3 equiv) and Xantphos Pd 4G (9.40 mg, 0.010 mmol, 0.1 equiv) in dioxane (1 mL) was stirred for overnight at 40° C. under carbon monoxide atmosphere. The residue was purified by silica gel column chromatography, eluted with PE/EA (1:1) to afford (2-(4-fluorophenyl)-1-methyl-1H-imidazo[4,5-b]pyrazin-6-yl)((1R,5S,6r)-6-(((6-(trifluoromethyl)pyridin-2-yl)oxy)methyl)-3-azabicyclo[3.1.0]hexan-3-yl)methanone. The residue was purified by reverse flash chromatography with the following conditions: column, C18 silica gel; mobile phase, MeCN in water, 10% to 100% gradient in 30 min; detector, UV 254 nm. This provided (2-(4-fluorophenyl)-1-methyl-1H-imidazo[4,5-b]pyrazin-6-yl)((1R,5S,6r)-6-(((6-(trifluoromethyl)pyridin-2-yl)oxy)methyl)-3-azabicyclo[3.1.0]hexan-3-yl)methanone (6 mg, 11.4%) as a white solid. ¹H NMR (300 MHz, DMSO-d₆) δ 8.85 (s, 1H), 8.15-8.05 (m, 2H), 7.96 (t, J=7.9 Hz, 1H), 7.55-7.42 (m, 3H), 7.14 (d, J=8.4 Hz, 1H), 4.23 (dd, J=7.2, 1.3 Hz, 2H), 4.05-3.92 (m, 6H), 3.60 (dd, J=12.2, 3.3 Hz, 1H), 1.87-1.77 (m, 2H), 1.17-1.07 (m, 1H). MS m/z: 512.8 [M+H]⁺.

### 1-(4-(1-Methyl-5-((1R,5S,6r)-6-(((6-(trifluoromethyl)pyridin-2-yl)oxy)methyl)-3-azabicyclo[3.1.0]hexane-3-carbonyl)-1H-imidazo[4,5-b]pyrazin-2-yl)piperidin-1-yl)ethan-1-one (243)

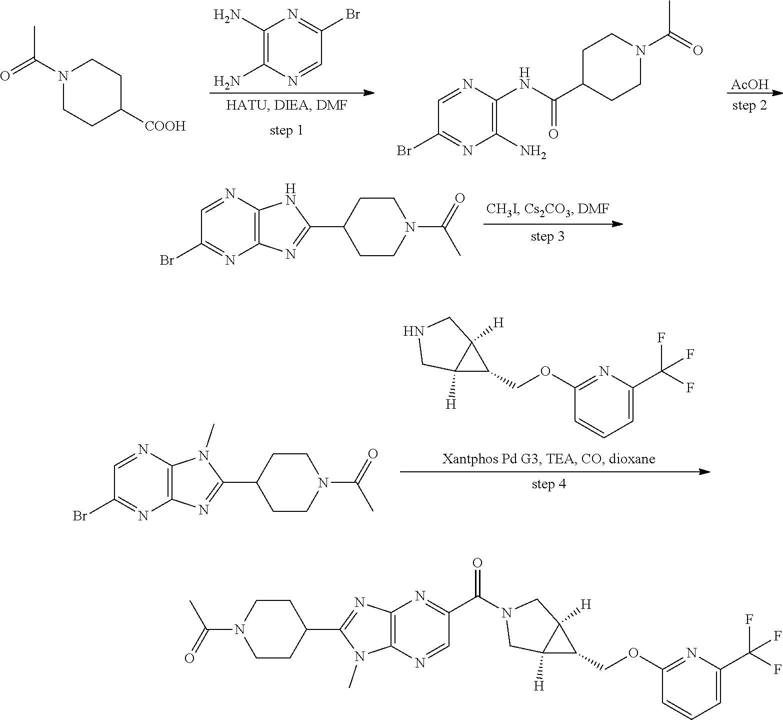

Step 1: 1-acetyl-N-(3-amino-5-bromopyrazin-2-yl)piperidine-4-carboxamide: A solution of 1-acetylpiperidine-4-carboxylic acid (1.5 g, 8.76 mmol, 1 equiv), 5-bromopyrazine-2,3-diamine (2.65 g, 14.0 mmol, 1.6 equiv), HATU (6.66 g, 17.5 mmol, 2 equiv) and DIPEA (3.40 g, 26.3 mmol, 3 equiv) in DMF (10 mL) was stirred for overnight at 70° C. The residue was purified by reverse flash chromatography with the following conditions: column, C18 silica gel; mobile phase, MeCN in water, 10% to 100% gradient in 30 min; detector, UV 254 nm. This provided 1-acetyl-N-(3-amino-5-bromopyrazin-2-yl)piperidine-4-carboxamide (1 g, 33.3%). MS m/z: 342 [M+H]⁺.

Step 2. 1-(4-(5-bromo-1H-imidazo[4,5-b]pyrazin-2-yl)piperidin-1-yl)ethan-1-one: A solution of 1-acetyl-N-(3-amino-5-bromopyrazin-2-yl)piperidine-4-carboxamide (1 g, 2.92 mmol, 1 equiv) in AcOH (10 mL) was stirred for overnight at 100° C. The resulting mixture was concentrated under vacuum. The residue was purified by reverse flash chromatography with the following conditions: column, C18 silica gel; mobile phase, MeCN in water, 10% to 100% gradient in 30 min; detector, UV 254 nm. This provided 1-(4-(5-bromo-1H-imidazo[4,5-b]pyrazin-2-yl)piperidin-1-yl)ethan-1-one (600 mg, 63.3%). MS m/z: 324 [M+H]⁺.

Step 3. 1-(4-(5-bromo-1-methyl-1H-imidazo[4,5-b]pyrazin-2-yl)piperidin-1-yl)ethan-1-one: A solution of 1-(4-(5-bromo-1H-imidazo[4,5-b]pyrazin-2-yl)piperidin-1-yl)ethan-1-one (600 mg, 1.85 mmol, 1 equiv), MeI (289 mg, 2.04 mmol, 1.1 equiv) and Cs₂CO₃ (1206 mg, 3.70 mmol, 2 equiv) in DMF (5 mL) was stirred for overnight at room temperature. The residue was purified by reverse flash chromatography with the following conditions: column, C18 silica gel; mobile phase, MeCN in water, 10% to 100% gradient in 30 min; detector, UV 254 nm. This provided 1-(4-(5-bromo-1-methyl-1H-imidazo[4,5-b]pyrazin-2-yl)piperidin-1-yl)ethan-1-one (350 mg, 55.91%). MS m/z: 338 [M+H]⁺.

Step 4. 1-(4-(1-methyl-5-((1R,5S,6r)-6-(((6-(trifluoromethyl)pyridin-2-yl)oxy)methyl)-3-azabicyclo[3.1.0]hexane-3-carbonyl)-1H-imidazo[4,5-b]pyrazin-2-yl)piperidin-1-yl)ethan-1-one: A solution of 1-(4-(5-bromo-1-methyl-1H-imidazo[4,5-b]pyrazin-2-yl)piperidin-1-yl)ethan-1-one (50 mg, 0.148 mmol, 1 equiv), (1R,5S,6S)-6-({[6-(trifluoromethyl)pyridin-2-yl]oxy}methyl)-3-azabicyclo[3.1.0]hexane (38.2 mg, 0.148 mmol, 1 equiv), Et₃N (44.9 mg, 0.444 mmol, 3 equiv) and Xantphos Pd G3 (14.2 mg, 0.015 mmol, 0.1 equiv) in dioxane (3 mL) was stirred for overnight at 50° C. under carbon monoxide atmosphere. The residue was purified by silica gel column chromatography, eluted with PE/EA (1:1) to afford impure product. This was purified by reverse flash chromatography with the following conditions: column, C18 silica gel; mobile phase, MeCN in water, 10% to 100% gradient in 30 min; detector, UV 254 nm. This provided 1-(4-(1-methyl-5-((1R,5S,6r)-6-(((6-(trifluoromethyl)pyridin-2-yl)oxy)methyl)-3-azabicyclo[3.1.0]hexane-3-carbonyl)-1H-imidazo[4,5-b]pyrazin-2-yl)piperidin-1-yl)ethan-1-one (30 mg, 36.8%) as a white solid. ¹H NMR (400 MHZ, DMSO-d₆) δ 8.66 (dd, J=2.5, 1.2 Hz, 1H), 7.98-7.91 (m, 1H), 7.47-7.42 (m, 1H), 7.17-7.11 (m, 1H), 4.46 (d, J=13.0 Hz, 1H), 4.28-4.14 (m, 2H), 4.02-3.92 (m, 3H), 3.91-3.77 (m, 4H), 3.59-3.51 (m, 1H), 3.49-3.42 (m, 1H), 3.32-3.21 (m, 1H), 2.87-2.74 (m, 1H), 2.08-1.95 (m, 5H), 1.89-1.75 (m, 3H), 1.71-1.64 (m, 1H), 1.14-1.05 (m 1H). MS m/z: 544.1 [M+H]⁺.

### 1-(4-(1-Methyl-6-((1R,5S,6r)-6-(((6-(trifluoromethyl)pyridin-2-yl)oxy)methyl)-3-azabicyclo[3.1.0]hexane-3-carbonyl)-1H-imidazo[4,5-b]pyrazin-2-yl)piperidin-1-yl)ethan-1-one (244)

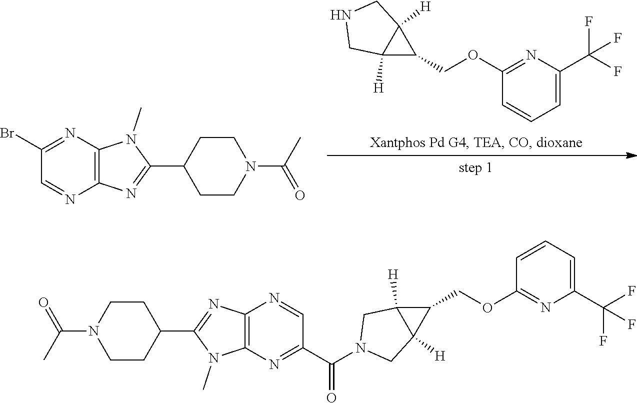 [SMILES: CC(=O)N1CCC(c2nc3ncc(Br)nc3n2C)CC1 ; FC(F)(F)c1cccc(OC[C@H]2[C@@H]3CNC[C@@H]32)n1 ; CC(=O)N1CCC(c2nc3ncc(C(=O)N4C[C@@H]5[C@@H](COc6cccc(C(F)(F)F)n6)[C@@H]5C4)nc3n2C)CC1]

A solution of 1-(4-{6-bromo-1-methylimidazo[4,5-b]pyrazin-2-yl}piperidin-1-yl)ethanone (50 mg, 0.148 mmol, 1 equiv), Et₃N (44.9 mg, 0.444 mmol, 3 equiv), Xantphos Pd G4 (14.2 mg, 0.015 mmol, 0.1 equiv) and (1R,5S,6S)-6-({[6-(trifluoromethyl)pyridin-2-yl]oxy}methyl)-3-azabicyclo[3.1.0]hexane (38.2 mg, 0.148 mmol, 1 equiv) in dioxane (3 mL) was stirred for overnight at 50° C. under carbon monoxide atmosphere. The resulting mixture was concentrated under reduced pressure. The residue was purified by silica gel column chromatography, eluted with PE/EA (1:1) to afford impure product. This was purified by reverse flash chromatography with the following conditions: column, C18 silica gel; mobile phase, MeCN in water, 10% to 100% gradient in 30 min; detector, UV 254 nm. This provided 1-(4-(1-methyl-6-((1R,5S,6r)-6-(((6-(trifluoromethyl)pyridin-2-yl)oxy)methyl)-3-azabicyclo[3.1.0]hexane-3-carbonyl)-1H-imidazo[4,5-b]pyrazin-2-yl)piperidin-1-yl)ethan-1-one (30 mg, 37.3%) as a white solid. ¹H NMR (400 MHZ, DMSO-d₆) δ 8.74 (s, 1H), 7.95 (t, J=8.0 Hz, 1H), 7.46 (dd, J=7.8, 3.0 Hz, 1H), 7.13 (dd, J=8.5, 2.5 Hz, 1H), 4.52-4.40 (m, 1H), 4.28-4.13 (m, 2H), 4.08-3.90 (m, 4H), 3.88 (s, 3H), 3.61-3.53 (m, 1H), 3.52-3.43 (m, 1H), 3.31-3.21 (m, 1H), 2.87-2.71 (m, 1H), 2.06 (s, 3H), 2.03-1.94 (m, 2H), 1.90-1.75 (m, 3H), 1.69-1.57 (m, 1H), 1.14-1.04 (m, 1H). MS m/z: 544.2 [M+H]⁺.

### 1-(4-(5-((1R,5S,6r)-6-((3,5-Difluorophenoxy)methyl)-3-azabicyclo[3.1.0]hexane-3-carbonyl)-1-methyl-1H-imidazo[4,5-b]pyrazin-2-yl)piperidin-1-yl)ethan-1-one (245)

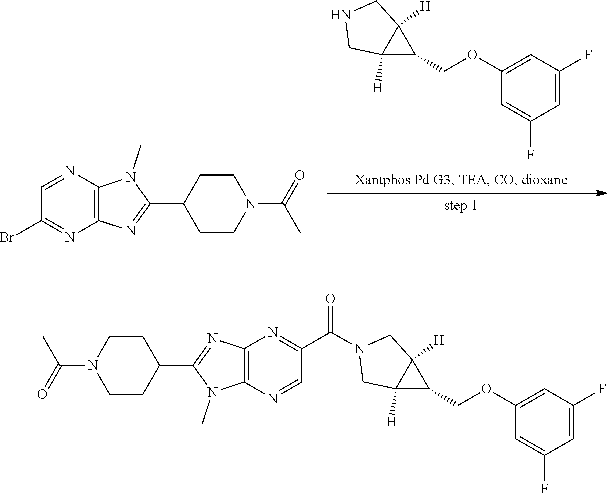 [SMILES: CC(=O)N1CCC(c2nc3nc(Br)cnc3n2C)CC1 ; Fc1cc(F)cc(OC[C@H]2[C@@H]3CNC[C@@H]32)c1 ; CC(=O)N1CCC(c2nc3nc(C(=O)N4C[C@@H]5[C@@H](COc6cc(F)cc(F)c6)[C@@H]5C4)cnc3n2C)CC1]

A solution of 1-(4-{5-bromo-1-methylimidazo[4,5-b]pyrazin-2-yl}piperidin-1-yl)ethanone (50 mg, 0.148 mmol, 1 equiv), (1R,5S,6S)-6-(3,5-difluorophenoxymethyl)-3-azabicyclo[3.1.0]hexane (33.3 mg, 0.148 mmol, 1 equiv), Et₃N (44.9 mg, 0.444 mmol, 3 equiv) and Xantphos Pd G3 (14.2 mg, 0.015 mmol, 0.1 equiv) in dioxane (3 mL) was stirred for overnight at 50° C. under carbon monoxide atmosphere. The resulting mixture was concentrated under reduced pressure. The residue was purified by silica gel column chromatography, eluted with PE/EA (1:1) to afford impure product. This was purified by reverse flash chromatography with the following conditions: column, C18 silica gel; mobile phase, MeCN in water, 10% to 100% gradient in 30 min; detector, UV 254 nm. This provided 1-(4-(5-((1R,5S,6r)-6-((3,5-difluorophenoxy)methyl)-3-azabicyclo[3.1.0]hexane-3-carbonyl)-1-methyl-1H-imidazo[4,5-b]pyrazin-2-yl)piperidin-1-yl)ethan-1-one (40 mg, 53.0%) as a white solid. ¹H NMR (400 MHZ, DMSO-d₆) δ 8.68 (s, 1H), 6.78-6.69 (m, 3H), 4.49-4.43 (m, 1H), 4.00-3.82 (m, 10H), 3.60-3.44 (m, 3H), 3.43-3.31 (m, 2H), 2.85-2.71 (m, 1H), 2.06-1.84 (m, 5H), 1.83-1.60 (m, 4H), 1.11-1.04 (m, 1H). MS m/z: 510.9 [M+H]⁺.

### 1-(4-(6-((1R,5S,6r)-6-((3,5-Difluorophenoxy)methyl)-3-azabicyclo[3.1.0]hexane-3-carbonyl)-1-methyl-1H-imidazo[4,5-b]pyrazin-2-yl)piperidin-1-yl)ethan-1-one (246)

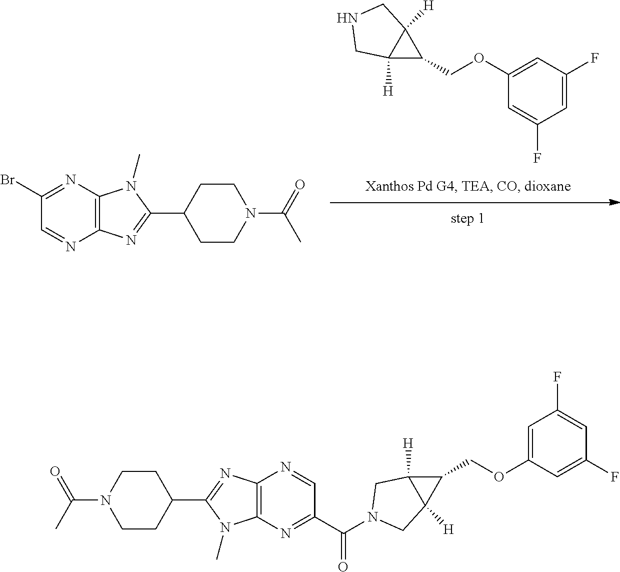 [SMILES: CC(=O)N1CCC(c2nc3ncc(Br)nc3n2C)CC1 ; Fc1cc(F)cc(OC[C@H]2[C@@H]3CNC[C@@H]32)c1 ; CC(=O)N1CCC(c2nc3ncc(C(=O)N4C[C@@H]5[C@@H](COc6cc(F)cc(F)c6)[C@@H]5C4)nc3n2C)CC1]

A solution of 1-(4-{6-bromo-1-methylimidazo[4,5-b]pyrazin-2-yl}piperidin-1-yl)ethanone (50 mg, 0.148 mmol, 1 equiv), (1R,5S,6S)-6-(3,5-difluorophenoxymethyl)-3-azabicyclo[3.1.0]hexane (33.3 mg, 0.148 mmol, 1 equiv), Et₃N (44.9 mg, 0.444 mmol, 3 equiv), and Xantphos Pd G4 (14.2 mg, 0.015 mmol, 0.1 equiv) in dioxane (3 mL) was stirred for overnight at 50° C. under carbon monoxide atmosphere. The resulting mixture was concentrated under reduced pressure. The residue was purified by silica gel column chromatography, eluted with PE/EA (1:1) to afford impure product. The crude product was purified by Prep-HPLC to afford 1-(4-(5-((1R,5S,6r)-6-((3,5-difluorophenoxy)methyl)-3-azabicyclo[3.1.0]hexane-3-carbonyl)-1-methyl-1H-imidazo[4,5-b]pyrazin-2-yl)piperidin-1-yl)ethan-1-one (30 mg, 39.0%) as a white solid. ¹H NMR (400 MHZ, DMSO-d₆) δ 8.76 (s. 1H), 6.78-6.68 (m, 3H), 4.49-4.46 (m, 1H), 4.02-3.88 (m, 9H), 3.60-3.55 (m, 1H), 3.48-3.42 (m, 1H), 3.33-3.24 (m, 1H), 2.80-2.70 (m, 1H), 2.07-1.96 (m, 5H), 1.87-1.82 (m, 1H), 1.73-1.63 (m, 3H), 1.10-1.03 (m, 1H). MS m/z: 510.9 [M+H]⁺.

### (1R,5S,6S)-3-[5-(1,3,4-Thiadiazol-2-yl)pyrazine-2-carbonyl]-6-({[6-(trifluoromethyl)pyridin-2-yl]oxy}methyl)-3-azabicyclo[3.1.0]hexane (247)

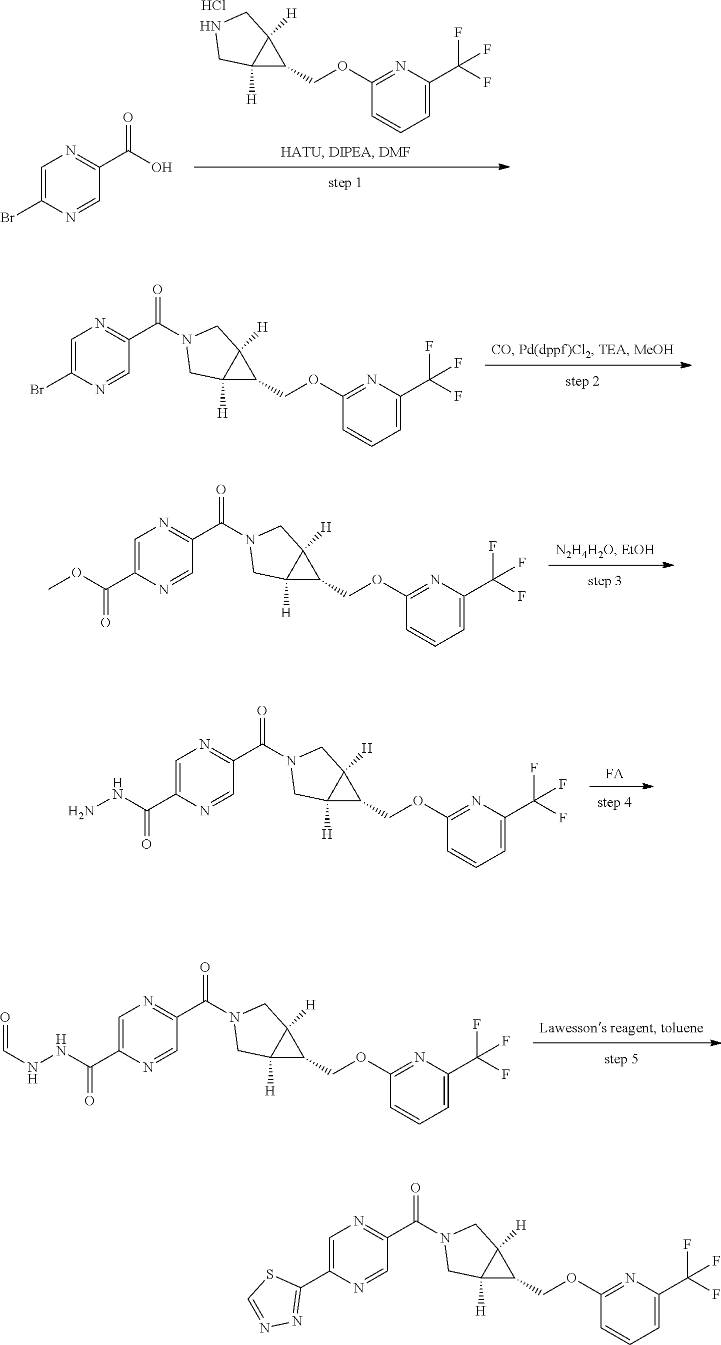

Step 1: (1R,5S,6S)-3-(5-bromopyrazine-2-carbonyl)-6-({[6-(trifluoromethyl)pyridin-2-yl]oxy}methyl)-3-azabicyclo[3.1.0]hexane: Followed General Procedure E using 5-bromopyrazine-2-carboxylic acid (120 mg, 0.591 mmol, 1.00 equiv) and (1R,5S,6S)-6-({[6-(trifluoromethyl)pyridin-2-yl]oxy}methyl)-3-azabicyclo[3.1.0]hexane hydrochloride (174 mg, 0.591 mmol, 1.00 equiv) to afford (1R,5S,6S)-3-(5-bromopyrazine-2-carbonyl)-6-({[6-(trifluoromethyl)pyridin-2-yl]oxy}methyl)-3-azabicyclo[3.1.0]hexane (160 mg, 61.0%) as a light yellow solid. MS m/z: 443 [M+H]⁺.

Step 2: methyl 5-[(1R,5S,6S)-6-({[6-(trifluoromethyl)pyridin-2-yl]oxy}methyl)-3-azabicyclo-[3.1.0]hexane-3-carbonyl]pyrazine-2-carboxylate: To a stirred solution of (1R,5S,6S)-3-(5-bromopyrazine-2-carbonyl)-6-({[6-(trifluoromethyl)pyridin-2-yl]oxy}methyl)-3-azabicyclo[3.1.0]hexane (160 mg, 0.361 mmol, 1.00 equiv) in MeOH (8 mL) were added Pd(dppf)Cl₂ (26.4 mg, 0.036 mmol, 0.10 equiv) and TEA (109.59 mg, 1.083 mmol, 3.00 equiv) at 0° C. The resulting mixture was stirred for overnight at 100° C. under carbon monoxide atmosphere (30 atm). The resulting mixture was diluted with water (20 mL). The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (1:1) to afford methyl 5-[(1R,5S,6S)-6-({[6-(trifluoromethyl)pyridin-2-yl]oxy}methyl)-3-azabicyclo[3.1.0]hexane-3-carbonyl]pyrazine-2-carboxylate (140 mg, 91.8%) as a light yellow oil. MS m/z: 423 [M+H]⁺.

Step 3: 5-[(1R,5S,6S)-6-({[6-(trifluoromethyl)pyridin-2-yl]oxy}methyl)-3-azabicyclo[3.1.0]-hexane-3-carbonyl]pyrazine-2-carbohydrazide: To a stirred solution of methyl 5-[(1R,5S,6S)-6-({[6-(trifluoromethyl)pyridin-2-yl]oxy}methyl)-3-azabicyclo[3.1.0]hexane-3-carbonyl]pyrazine-2-carboxylate (140 mg, 0.331 mmol, 1.00 equiv) in EtOH (2 mL) was added NH₂NH₂H₂O (33.19 mg, 0.662 mmol, 2.00 equiv) at room temperature. The resulting mixture was stirred for 2 h at 80° C. The resulting mixture was concentrated under vacuum. This provided 5-[(1R,5S,6S)-6-({[6-(trifluoromethyl)pyridin-2-yl]oxy}methyl)-3-azabicyclo[3.1.0]hexane-3-carbonyl]pyrazine-2-carbohydrazide (120 mg, crude) as a light yellow solid. MS m/z: 423 [M+H]⁺.

Step 4: N′-formyl-5-[(1R,5S,6S)-6-({[6-(trifluoromethyl)pyridin-2-yl]oxy}methyl)-3-azabicyclo[3.1.0]hexane-3-carbonyl]pyrazine-2-carbohydrazide: 5-[(1R,5S,6S)-6-({[6-(trifluoromethyl)pyridin-2-yl]oxy}methyl)-3-azabicyclo[3.1.0]hexane-3-carbonyl]-pyrazine-2-carbohydrazide (120 mg, 0.284 mmol, 1.00 equiv) was dissolved in HCOOH (2 mL) at room temperature. The resulting solution was stirred for 2 h at 80° C. The resulting mixture was concentrated under vacuum. The residue was purified by reverse flash with the following conditions: column, C18 gel; mobile phase, MeCN in water (0.1% FA), 20% to 60% gradient in 16 min; detector, UV 254 nm. This provided N′-formyl-5-[(1R,5S,6S)-6-({[6-(trifluoromethyl)pyridin-2-yl]oxy}methyl)-3-azabicyclo[3.1.0]hexane-3-carbonyl]pyrazine-2-carbohydrazide (100 mg, 78.1%) as a light yellow solid. MS m/z: 451 [M+H]⁺.

Step 5: (1R,5S,6S)-3-[5-(1,3,4-thiadiazol-2-yl)pyrazine-2-carbonyl]-6-({[6-(trifluoromethyl)-pyridin-2-yl]oxy}methyl)-3-azabicyclo[3.1.0]hexane: To a stirred solution of N′-formyl-5-[(1R,5S,6S)-6-({[6-(trifluoromethyl)pyridin-2-yl]oxy}methyl)-3-azabicyclo[3.1.0]hexane-3-carbonyl]pyrazine-2-carbohydrazide (30 mg, 0.067 mmol, 1.00 equiv) in toluene (2 mL) was added Lawesson reagent (21.55 mg, 0.054 mmol, 0.80 equiv) at room temperature. The resulting mixture was stirred for 2 h at 80° C. The reaction was quenched with sat. NaHCO₃(aq.) at 0° C. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by reverse flash with the following conditions: column, C18 gel; mobile phase, MeCN in water (0.1% FA), 30% to 60% gradient in 16 min; detector, UV 254 nm. This provided (1R,5S,6S)-3-[5-(1,3,4-thiadiazol-2-yl)pyrazine-2-carbonyl]-6-({[6-(trifluoromethyl)pyridin-2-yl]oxy}methyl)-3-azabicyclo[3.1.0]hexane (17 mg, 56.91%) as a light yellow solid. ¹H NMR (400 MHZ, CD₃OD): δ 9.61 (s, 1H), 9.52 (d, J=1.6 Hz, 1H), 9.04 (d, J=1.6 Hz, 1H), 7.84 (t, J=8.0 Hz, 1H), 7.32 (d, J=7.2 Hz, 1H), 7.00 (d, J=8.0 Hz, 1H), 4.33-4.26 (m, 2H), 4.14-3.97 (m, 3H), 3.68 (dd, J=12.0, 3.6 Hz, 1H), 1.88-1.80 (m, 2H), 1.23-1.15 (m, 1H). MS m/z: 449.0 [M+H]⁺.

### 1-(2-(2,2,2-Trifluoroethoxy)ethyl)-6-(3-(((2-(trifluoromethyl)pyridin-3-yl)oxy)methyl)piperidin-1-yl)-1H-pyrazolo[3,4-b]pyrazine (248)

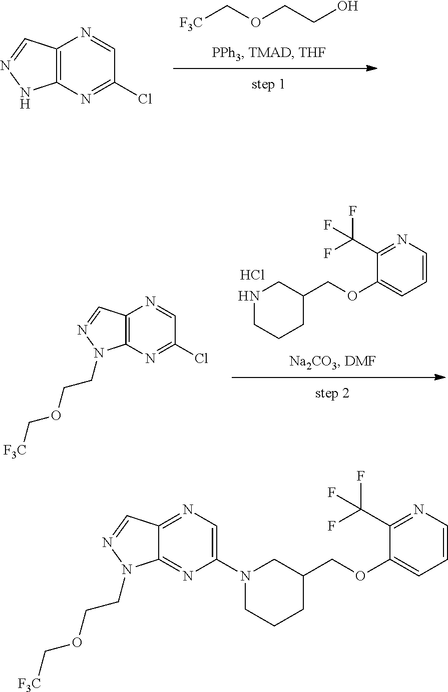 [SMILES: OCCOCC(F)(F)F ; Clc1cnc2cn[nH]c2n1 ; FC(F)(F)COCCn1ncc2ncc(Cl)nc21 ; Cl ; FC(F)(F)c1ncccc1OCC1CCCNC1 ; FC(F)(F)COCCn1ncc2ncc(N3CCCC(COc4cccnc4C(F)(F)F)C3)nc21]

Step 1: 6-chloro-1-(2-(2,2,2-trifluoroethoxy)ethyl)-1H-pyrazolo[3,4-b]pyrazine: Followed General Procedure A using 6-chloro-1H-pyrazolo[3,4-b]pyrazine (200 mg, 1.29 mmol, 1 equiv) and 2-(2,2,2-trifluoroethoxy)ethan-1-ol (279 mg, 1.93 mmol, 1.5 equiv) to afford 6-chloro-1-(2-(2,2,2-trifluoroethoxy)ethyl)-1H-pyrazolo[3,4-b]pyrazinee (80 mg, 22.1%) as a yellow solid. MS m/z: 281 [M+H]⁺.

Step 2: 3-(phenoxymethyl)-1-(1-phenyl-1H-1,2,3-triazol-4-yl)piperidine: Followed General Procedure C using 3-(piperidin-3-ylmethoxy)-2-(trifluoromethyl)pyridine hydrochloride (80.0 mg, 0.270 mmol, 1.00 equiv) and 6-chloro-1-(2-(2,2,2-trifluoroethoxy)ethyl)-1H-pyrazolo[3,4-b]pyrazinee (75.6 mg, 0.270 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2-(2,2,2-trifluoroethoxy)ethyl)-6-(3-(((2-(trifluoromethyl)pyridin-3-yl)oxy)methyl)piperidin-1-yl)-1H-pyrazolo[3,4-b]pyrazine (45 mg, 33.0%) as a white solid. ¹H NMR (400 MHZ, DMSO-d₆) δ 8.39 (s, 1H), 8.27 (dd, J=4.5, 1.1 Hz, 1H), 8.06 (s, 1H), 7.82 (d, J=8.5 Hz, 1H), 7.73-7.66 (m, 1H), 4.68-4.60 (m, 1H), 4.50-4.33 (m, 3H), 4.25-4.16 (m, 1H), 4.15-3.93 (m, 5H), 3.19-3.07 (m, 1H), 3.00 (dd, J=13.2, 10.3 Hz, 1H), 2.15-2.08 (m, 1H), 1.91 (dd, J=12.5, 3.7 Hz, 1H), 1.86-1.76 (m, 1H), 1.64-1.32 (m, 2H). MS m/z: 505.0 [M+H]⁺.

### (1-(2-Ethoxyethyl)-1H-pyrazolo[3,4-b]pyrazin-6-yl)(3-(((2-(trifluoromethyl)pyridin-3-yl)oxy)methyl)piperidin-1-yl)methanone (249)

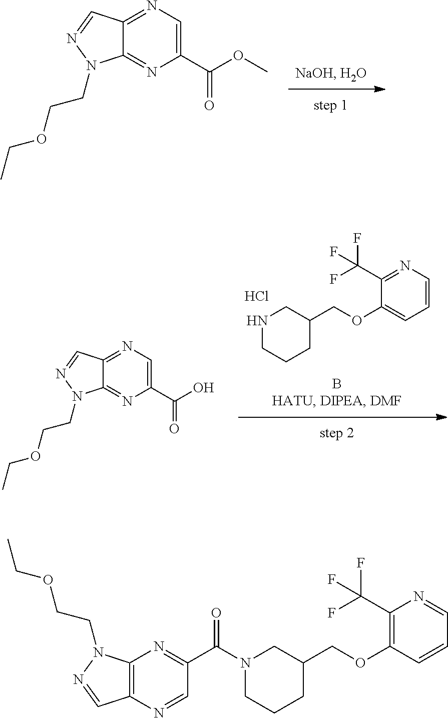 [SMILES: CCOCCn1ncc2ncc(C(=O)OC)nc21 ; CCOCCn1ncc2ncc(C(=O)O)nc21 ; Cl ; FC(F)(F)c1ncccc1OCC1CCCNC1 ; CCOCCn1ncc2ncc(C(=O)N3CCCC(COc4cccnc4C(F)(F)F)C3)nc21]

Step 1: 1-(2-ethoxyethyl)-1H-pyrazolo[3,4-b]pyrazine-6-carboxylic acid: To a stirred solution of methyl 1-(2-ethoxyethyl)-1H-pyrazolo[3,4-b]pyrazine-6-carboxylate (50 mg, 0.200 mmol, 1 equiv) and NaOH (32.0 mg, 0.80 mmol, 4 equiv) in H₂O (1 mL, 55.5 mmol) and MeOH (1 mL). The resulting mixture was stirred for 2 h at room temperature. The reaction was monitored by LCMS. The resulting mixture was purified by reverse flash chromatography with the following conditions: column, C18 silica gel; mobile phase, ACN in water, 5% to 100% gradient in 20 min; detector, UV 254/220 nm to afford 1-(2-ethoxyethyl)-1H-pyrazolo[3,4-b]pyrazine-6-carboxylic acid (30 mg, 63.5%) as a white solid. MS m/z: 237 [M+H]⁺.

Step 2: (1-(2-ethoxyethyl)-1H-pyrazolo[3,4-b]pyrazin-6-yl)(3-(((2-(trifluoromethyl)pyridin-3-yl)oxy)methyl)piperidin-1-yl)methanone: Followed General Procedure E using 1-(2-ethoxyethyl)-1H-pyrazolo[3,4-b]pyrazine-6-carboxylic acid (30 mg, 0.127 mmol, 1 equiv) and 3-(piperidin-3-ylmethoxy)-2-(trifluoromethyl)pyridine hydrochloride (41.4 mg, 0.140 mmol, 1.1 equiv). The crude product was purified by reverse flash chromatography with the following conditions: column, C18 silica gel; mobile phase, ACN in water, 5% to 100% gradient in 20 min; detector, UV 254/220 nm to afford 3-({1-[1-(2-ethoxyethyl)pyrazolo[3,4-b]pyrazine-6-carbonyl]piperidin-3-yl}methoxy)-2-(trifluoromethyl)pyridine (55.3 mg, 89.5%) as light yellow semi-solid. ¹H NMR (300 MHZ, DMSO-d₆) δ 8.84-8.69 (m, 1H), 8.59-8.54 (m, 1H), 8.23 (dd, J=30.0, 4.4 Hz, 1H), 7.87-7.59 (m, 2H), 4.67-4.59 (m, 1H), 4.58-4.48 (m, 1H), 4.37-4.04 (m, 2H), 3.93-3.63 (m, 4H), 3.27 (d, J=0.9 Hz, 2H), 3.17-2.87 (m, 2H), 2.29-1.79 (m, 3H), 1.69-1.40 (m, 2H), 0.96-0.82 (m, 3H). MS m/z: 479.1 [M+H]⁺.

### 3-({1-[3-(2-ethoxyethyl)imidazo[4,5-b]pyrazine-6-carbonyl]piperidin-3-yl}methoxy)-2-(trifluoromethyl)pyridine (250)

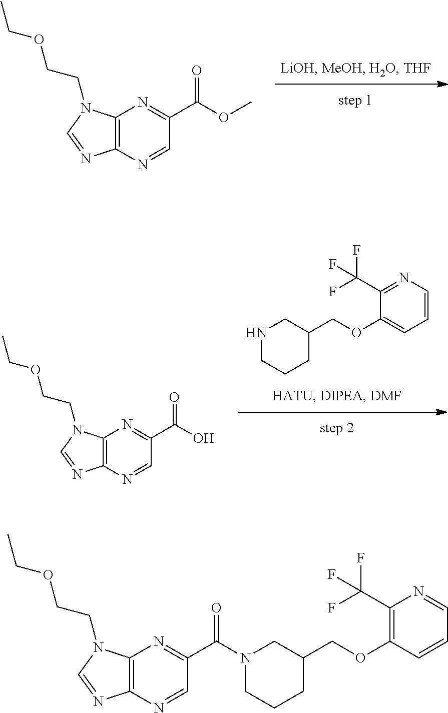 [SMILES: CCOCCn1cnc2ncc(C(=O)OC)nc21 ; CCOCCn1cnc2ncc(C(=O)O)nc21 ; FC(F)(F)c1ncccc1OCC1CCCNC1 ; CCOCCn1cnc2ncc(C(=O)N3CCCC(COc4cccnc4C(F)(F)F)C3)nc21]

Step 1: 1-(2-ethoxyethyl)-1H-imidazo[4,5-b]pyrazine-6-carboxylic acid: To a stirred solution of methyl 1-(2-ethoxyethyl)-1H-imidazo[4,5-b]pyrazine-6-carboxylate (50 mg, 0.2 mmol, 1 equiv) in MeOH (0.5 mL), THF (0.5 mL) and H₂O (0.5 mL) was added LiOH·H₂O (16.8 mg, 0.4 mmol, 2 equiv) at 0° C. The resulting mixture was stirred for 2 h at 0° C. under. The resulting mixture was acidified by HCl (3 M) to PH ˜3. The aqueous phase was extracted by EtOAc (3×10 mL). The combined organic layers were washed by water (2×15 mL) and brine (1×15 mL), dried over anhydrous Na₂SO₄. After filtration, the resulting mixture was concentrated under reduced pressure to afford 1-(2-ethoxyethyl)-1H-imidazo[4,5-b]pyrazine-6-carboxylic acid (50 mg, crude) as a white solid. MS m/z: 237 [M+H]⁺.

Step 2: (1-(2-ethoxyethyl)-1H-imidazo[4,5-b]pyrazin-6-yl)(3-(((2-(trifluoromethyl)pyridin-3-yl)oxy)methyl)piperidin-1-yl)methanone: Followed General Procedure E using 1-(2-ethoxyethyl)-1H-imidazo[4,5-b]pyrazine-6-carboxylic acid (50 mg, 0.212 mmol, 1 equiv) and 3-(piperidin-3-ylmethoxy)-2-(trifluoromethyl)pyridine (66.2 mg, 0.254 mmol, 1.2 equiv). The crude product was purified by reverse flash chromatography with the following conditions: column, silica gel; mobile phase, MeCN in water, 10% to 50% gradient in 10 min; detector, UV 254 nm to afford (1-(2-ethoxyethyl)-1H-imidazo[4,5-b]pyrazin-6-yl)(3-(((2-(trifluoromethyl)pyridin-3-yl)oxy)methyl)piperidin-1-yl)methanone (23.8 mg, 23.5%) as a light yellow solid. ¹H NMR (400 MHZ, DMSO-d₆) δ 8.81 (s, 1H), 8.71 (m, 1H), 8.17 (m, 1H), 7.79-7.53 (m, 2H), 4.70-4.43 (m, 3H), 4.22-3.75 (m, 5H), 3.44 (m, 2H), 3.29-3.05 (m, 2H), 2.23 (m, 1H), 2.11-1.48 (m, 4H), 1.12-1.00 (m, 3H). MS m/z: 479.1 [M+H]⁺.

### (1-(2-Ethoxyethyl)-1H-imidazo[4,5-b]pyrazin-5-yl)(3-(((2-(trifluoromethyl)pyridin-3-yl)oxy)methyl)piperidin-1-yl)methanone (251)

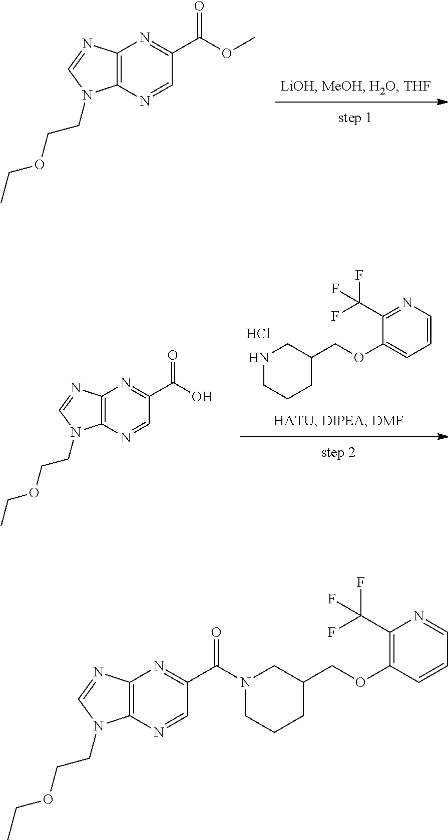 [SMILES: CCOCCn1cnc2nc(C(=O)OC)cnc21 ; CCOCCn1cnc2nc(C(=O)O)cnc21 ; Cl ; FC(F)(F)c1ncccc1OCC1CCCNC1 ; CCOCCn1cnc2nc(C(=O)N3CCCC(COc4cccnc4C(F)(F)F)C3)cnc21]

Step 1: 1-(2-ethoxyethyl)-1H-imidazo[4,5-b]pyrazine-5-carboxylic acid: To a stirred solution of methyl 1-(2-ethoxyethyl)-1H-imidazo[4,5-b]pyrazine-5-carboxylic acid (50 mg, 0.2 mmol, 1 equiv) in MeOH (0.5 mL), THF (0.5 mL) and H₂O (0.5 mL) was added LiOH·H₂O (16.8 mg, 0.4 mmol, 2 equiv) at 0° C. The resulting mixture was stirred for 2 h at 0° C. under. The resulting mixture was stirred for 2 h at 0° C. under. The resulting mixture was acidified by HCl (3 M) to PH ˜3. The aqueous phase was extracted by EtOAc (3×10 mL). The combined organic layers were washed by water (2×15 mL) and brine (1×15 mL), dried over anhydrous Na₂SO₄. After filtration, the resulting mixture was concentrated under reduced pressure to afford 1-(2-ethoxyethyl)-1-imidazo[4,5-b]pyrazine-6-carboxylic acid (25 mg, crude) as a white solid. MS m/z: 237 [M+H]⁺.

Step 2: (1-(2-ethoxyethyl)-1H-imidazo[4,5-b]pyrazin-5-yl)(3-(((2-(trifluoromethyl)pyridin-3-yl)oxy)methyl)piperidin-1-yl)methanone: Followed General Procedure E using 1-(2-ethoxyethyl)-1H-imidazo[4,5-b]pyrazine-5-carboxylic acid (50 mg, 0.212 mmol, 1 equiv) and 3-(piperidin-3-ylmethoxy)-2-(trifluoromethyl)pyridine (66.2 mg, 0.254 mmol, 1.2 equiv). The crude product was purified by reverse flash chromatography with the following conditions: column, silica gel; mobile phase, MeCN in water, 10% to 50% gradient in 10 min; detector, UV 254 nm to afford (1-(2-ethoxyethyl)-1H-imidazo[4,5-b]pyrazin-5-yl)(3-(((2-(trifluoromethyl)pyridin-3-yl)oxy)methyl)piperidin-1-yl)methanone (20.7 mg, 20.4%) as a light yellow solid. ¹H NMR (400 MHZ, Methanol-d₄) δ 8.84-8.72 (m, 1H), 8.70-8.53 (m, 1H), 8.26-8.10 (m, 1H), 7.80-7.54 (m, 2H), 4.72-4.55 (m, 3H), 4.42-4.04 (m, 2H), 3.96-3.84 (m, 3H), 3.57-3.47 (m, 2H), 3.30-3.09 (m, 2H), 2.31-2.16 (m, 1H), 2.08-1.52 (m, 4H), 1.20-1.08 (m, 3H). MS m/z: 479.1 [M+H]⁺.

### (1H-Indol-6-yl)((3S,5R)-3-methyl-5-(((2-(trifluoromethyl)pyridin-3yl)oxy)methyl)piperidin-1-yl)methanone(252a) and (1H-indol-6-yl)((3S,5S)-3-methyl-5-(((2-(trifluoromethyl)pyridin-3-yl)oxy)methyl)piperidin-1-yl)methanone (252b)

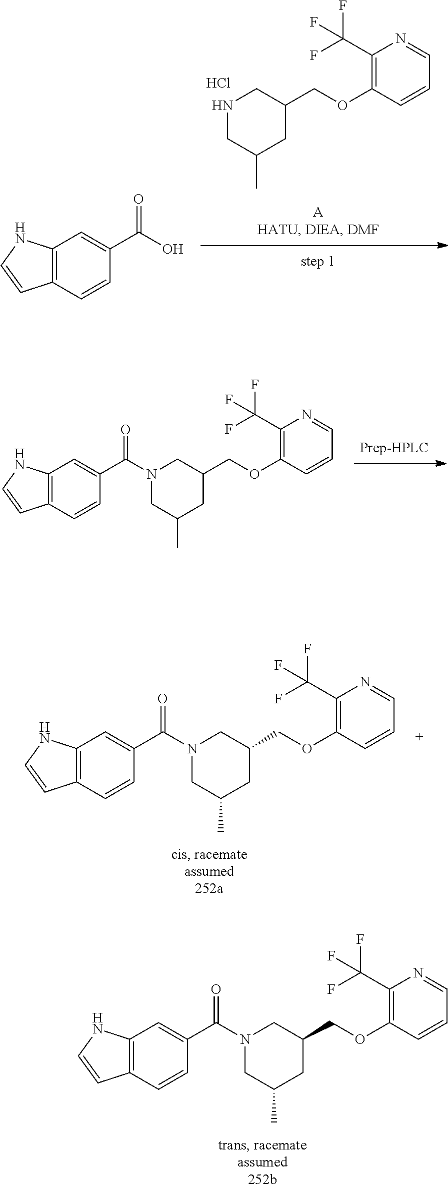 [SMILES: Cl ; CC1CNCC(COc2cccnc2C(F)(F)F)C1 ; O=C(O)c1ccc2cc[nH]c2c1 ; CC1CC(COc2cccnc2C(F)(F)F)CN(C(=O)c2ccc3cc[nH]c3c2)C1 ; C[C@H]1C[C@@H](COc2cccnc2C(F)(F)F)CN(C(=O)c2ccc3cc[nH]c3c2)C1 ; C[C@H]1C[C@H](COc2cccnc2C(F)(F)F)CN(C(=O)c2ccc3cc[nH]c3c2)C1]

Step 1: (1H-indol-6-yl)(3-methyl-5-(((2-(trifluoromethyl)pyridin-3-yl)oxy)methyl)piperidin-1-yl)methanone: Followed General Procedure E using 1H-indole-6-carboxylic acid (80 mg, 0.496 mmol, 1 equiv) and 3-((5-methylpiperidin-3-yl)methoxy)-2-(trifluoromethyl)pyridine hydrochloride (169.68 mg, 0.546 mmol, 1.1 equiv). The crude product was purified by reverse flash chromatography with the following conditions: column, C18 silica gel; mobile phase, MeCN in water, 5% to 100% gradient in 15 min; detector, UV 254 nm. This provided (1H-indol-6-yl)(3-methyl-5-(((2-(trifluoromethyl)pyridin-3-yl)oxy)methyl)piperidin-1-yl)methanone (80 mg) as a white solid. This product was further purified by prep. HPLC with the following conditions: Column: Xselect CSH F-Phenyl OBD Column 19*150 mm 5 μm, n; Mobile Phase A: Water (0.1% FA), Mobile Phase B: ACN; Flow rate: 25 mL/min; Gradient: 40% B to 55% B in 10 min, 55% B; Wavelength: 254/220 nm; RT1(min): 8.13. This provided(1H-indol-6-yl)((3S,5R)-3-methyl-5-(((2-(trifluoromethyl)pyridin-3-yl)oxy)methyl)piperidin-1-yl)methanone(252a, assumed structure, 39.0 mg, 19.6%) and (1H-indol-6-yl)((3S,5S)-3-methyl-5-(((2-(trifluoromethyl)pyridin-3-yl)oxy)methyl)piperidin-1-yl)methanone (252b, assumed structure, 13.6 mg, 18.6%) as white solids. 252a: ¹H NMR (400 MHZ, DMSO-d₆) δ 11.17 (s, 1H), 8.23 (d, J=4.5 Hz, 1H), 7.88-7.63 (m, 2H), 7.56-7.30 (m, 3H), 6.92 (s, 1H), 6.43 (s, 1H), 4.07 (s, 2H), 3.63 (s, 3H), 3.13 (s, 1H), 2.24 (s, 1H), 1.90 (s, 1H), 1.81-1.71 (m, 1H), 1.58-1.46 (m, 1H), 0.88 (s, 3H). MS m/z: 418.1[M+H]⁺. 252b: ¹H NMR (300 MHz, DMSO-d₆) δ 11.32 (s, 1H), 8.31 (d, J=4.5 Hz, 1H), 7.95-7.70 (d, J=22.5 Hz, 2H), 7.62 (d, J=8.1 Hz, 1H), 7.51 (d, J=3.3 Hz, 2H), 7.07 (d, J=8.1 Hz, 1H), 6.54 (d, J=2.6 Hz, 1H), 4.14 (s, 2H), 2.15 (s, 1H), 1.99 (d, J=12.7 Hz, 1H), 1.75 (s, 1H), 1.25-0.72 (m, 4H). MS m/z: 418.1 [M+H]⁺.

### 1-[5-Methyl-6-(2-methylphenyl)pyrrolo[2,3-b]pyrazine-7-carbonyl]-3-(2-methylphenoxymethyl)piperidine (253)

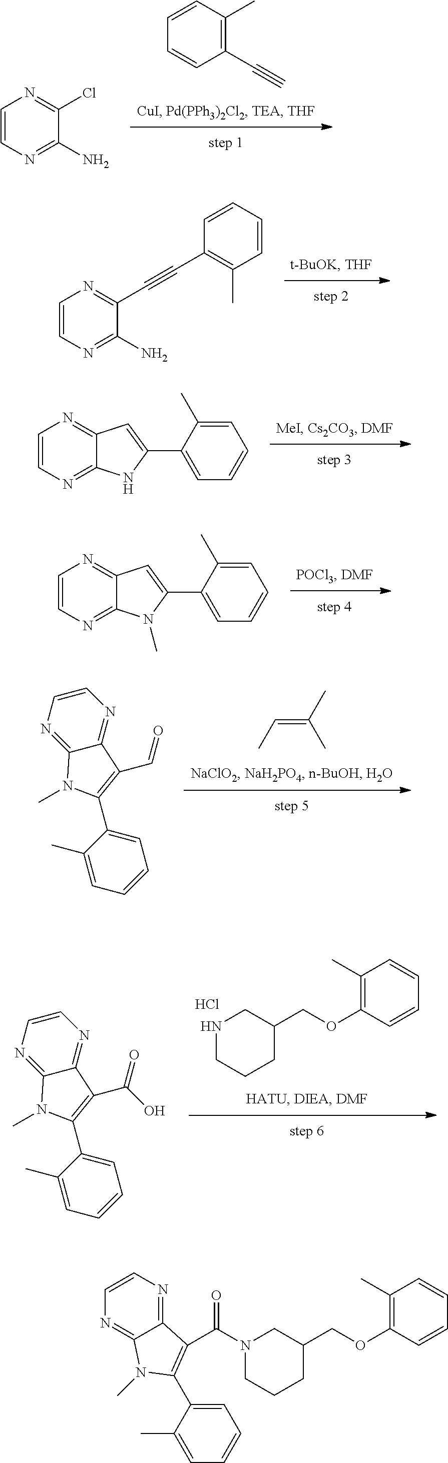

Step 1: 3-[2-(2-methylphenyl)ethynyl]pyrazin-2-amine: To a stirred solution of 3-chloropyrazin-2-amine (1 g, 7.72 mmol, 1 equiv) and 1-ethynyl-2-methylbenzene (1.08 g, 9.26 mmol, 1.2 equiv) in THF (10 mL) were added CuI (0.15 g, 0.772 mmol, 0.1 equiv) and Pd(PPh₃)₂Cl₂ (0.54 g, 0.772 mmol, 0.1 equiv) and TEA (3.22 mL, 23.157 mmol, 3.0 equiv). The resulting mixture was stirred for 3 h at 80° C. under nitrogen atmosphere. The resulting mixture was concentrated under vacuum. The resulting mixture was purified by silica gel column chromatography, eluted with PE/EA (1:1) to afford 3-[2-(2-methylphenyl)ethynyl]pyrazin-2-amine (1.12 g, 69.3%) as a brown solid. MS m/z: 210 [M+H]⁺.

Step 2: 6-phenyl-5H-pyrrolo[2,3-b]pyrazine: A solution of 3-(2-phenylethynyl)pyrazin-2-amine (1 g, 5.122 mmol, 1 equiv) and t-BuOK (0.86 g, 7.683 mmol, 1.5 equiv) in THF (10 mL) was stirred for 3 h at 80° C. The resulting mixture was diluted with EtOAc (50 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford 6-phenyl-5H-pyrrolo[2,3-b]pyrazine (400 mg, 40.0%) as a yellow solid. MS m/z: 210 [M+H]⁺.

Step 3: 5-methyl-6-(2-methylphenyl)pyrrolo[2,3-b]pyrazine): To a stirred solution of 6-(2-methylphenyl)-5H-pyrrolo[2,3-b]pyrazine (200 mg, 0.956 mmol, 1 equiv) and Cs₂CO₃ (934 mg, 2.868 mmol, 3 equiv) in DMF (2 mL) was added MeI (203 mg, 1.434 mmol, 1.5 equiv) dropwise at room temperature. The resulting mixture was stirred for 2 h at room temperature. Desired product could be detected by LCMS. The resulting mixture was diluted with EtOAc (30 mL). The organic layer was washed with water (3×15 mL), dried over anhydrous Na₂SO₄. After filtration, the filtrate was concentrated under reduced pressure. This provided 5-methyl-6-(2-methylphenyl)pyrrolo[2,3-b]pyrazine) as a yellow solid. MS m/z: 224 [M+H]⁺.

Step 4: 5-methyl-6-(2-methylphenyl)pyrrolo[2,3-b]pyrazine-7-carbaldehyde: A solution of 5-methyl-6-(2-methylphenyl)pyrrolo[2,3-b]pyrazine (200 mg, 0.896 mmol, 1 equiv) and POCl₃ (1 mL) in DMF (2 mL) was stirred for 12 h at 50° C. Desired product could be detected by LCMS. The resulting mixture was diluted with EtOAc (10 mL). The organic layer was washed with water (3×10 mL), dried over anhydrous Na₂SO₄. After filtration, the filtrate was concentrated under reduced pressure. This provided 5-methyl-6-(2-methylphenyl)pyrrolo[2,3-b]pyrazine-7-carbaldehyde (160 mg, 71.08%) as a brown solid. MS m/z: 252 [M+H]⁺.

Step 5: 5-methyl-6-(2-methylphenyl)pyrrolo[2,3-b]pyrazine-7-carboxylic acid: To a stirred solution of 5-methyl-6-(2-methylphenyl)pyrrolo[2,3-b]pyrazine-7-carbaldehyde (200 mg, 0.796 mmol, 1 equiv) and 2-methyl-2-butene (140 mg, 2.00 mmol, 2.5 equiv) in n-BuOH (2 mL)/H₂O (0.4 mL) were added NaH₂PO₄ (573 mg, 4.77 mmol, 6 equiv) and NaClO₂ (107.97 mg, 1.194 mmol, 1.5 equiv). The resulting mixture was stirred for 2 h at 50° C. The residue was purified by reverse flash chromatography with the following conditions: column, C18 silica gel; mobile phase, MeCN in water, 0% to 100% gradient in 30 min; detector, UV 254 nm. This provided 5-methyl-6-(2-methylphenyl)pyrrolo[2,3-b]pyrazine-7-carboxylic acid (150 mg, 70.5%) as a yellow solid. MS m/z: 268 [M+H]⁺.

Step 6: 1-[5-methyl-6-(2-methylphenyl)pyrrolo[2,3-b]pyrazine-7-carbonyl]-3-(2-methylphenoxymethyl)piperidine: To a stirred solution of 5-methyl-6-(2-methylphenyl)pyrrolo[2,3-b]pyrazine-7-carboxylic acid (100 mg, 0.374 mmol, 1.00 equiv) and HATU (156.48 mg, 0.411 mmol, 1.1 equiv) in DMF (1 mL) were added DIEA (145.06 mg, 1.122 mmol, 3 equiv) and 3-((o-tolyloxy)methyl)piperidine hydrochloride (99.3 mg, 0.411 mmol. 1.10 equiv) at 0° C. The resulting mixture was stirred for 2 h at room temperature. Desired product could be detected by LCMS. The residue was purified by reverse flash chromatography with the following conditions: column, silica gel; mobile phase, MeCN in water, 0% to 100% gradient in 20 min; detector, UV 254 nm. This provided 1-[5-methyl-6-(2-methylphenyl)pyrrolo[2,3-b]pyrazine-7-carbonyl]-3-(2-methylphenoxymethyl)piperidine (33 mg, 19.4%) as a light yellow solid. H NMR (400 MHZ, DMSO-d₆) δ 8.45 (s, 1H), 8.38 (s, 1H), 7.47-6.93 (m, 6H), 6.82-6.79 (m, 2H), 4.35-4.22 (m, 1H), 3.90-3.85 (m 2H), 3.50 (s, 4H), 2.97-2.84 (m, 2H), 2.20-2.07 (m, 4H), 1.89-1.80 (m, 2H), 1.69 (s, 1H), 1.38-1.23 (m, 4H). MS m/z: 455.2 [M+H]⁺.

### 1-[6-(2-Methoxyphenyl)-5-methylpyrrolo[2,3-b]pyrazine-7-carbonyl]-3-(2-methylphenoxymethyl)piperidine (254)

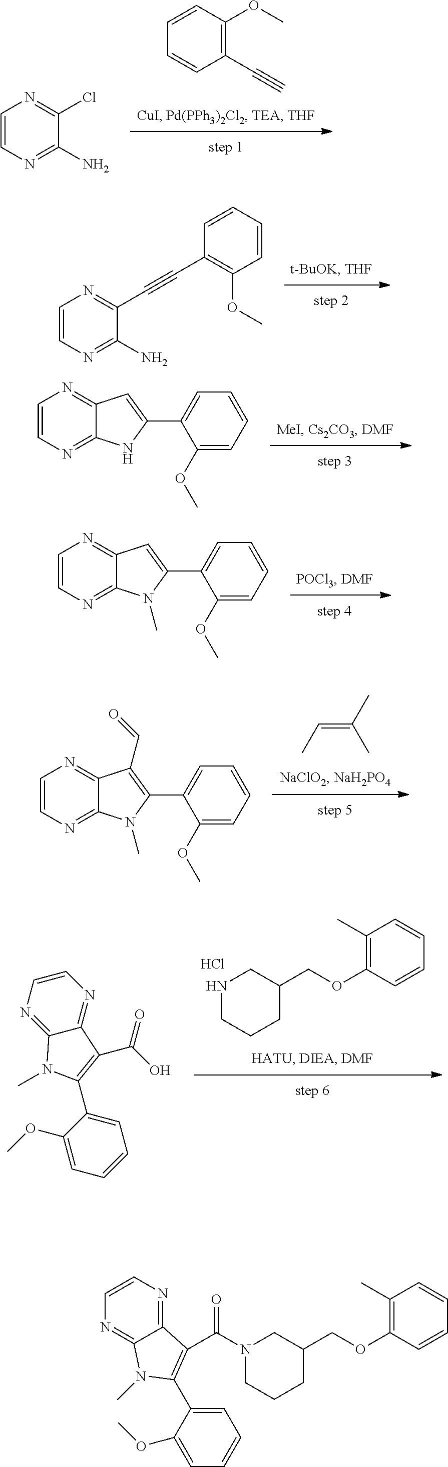

Step 1: 3-[2-(2-methoxyphenyl)ethynyl]pyrazin-2-amine: To a stirred solution of 3-chloropyrazin-2-amine (1 g, 7.7C mmol, 1 equiv) and 1-ethynyl-2-methoxybenzene (1.22 g, 9.26 mmol, 1.2 equiv) in THF (10 mL) were added CuI (0.15 g, 0.77 mmol, 0.1 equiv) and Pd(dppf)Cl₂ (0.56 g, 0.772 mmol, 0.1 equiv) and TEA (2.34 g, 23.1 mmol, 3 equiv). The resulting mixture was stirred for 3 h at 80° C. under nitrogen atmosphere. The resulting mixture was concentrated under vacuum. The residue was purified by silica gel column chromatography, eluted with PE/EA (1:1) to afford 3-[2-(2-methoxyphenyl)ethynyl]pyrazin-2-amine (700 mg, 40.26%) as a brown solid. MS m/z: 226 [M+H]⁺.

Step 2: 6-(2-methoxyphenyl)-5H-pyrrolo[2,3-b]pyrazine: A solution of 3-[2-(2-methoxyphenyl)ethynyl]pyrazin-2-amine (1.5 g, 6.66 mmol, 1 equiv) and t-BuOK (1.12 g, 9.99 mmol, 1.5 equiv) in THF (15 mL) was stirred for 13 h at 80° C. The resulting mixture was concentrated under vacuum. The residue was purified by silica gel column chromatography, eluted with PE/EA (1:1) to afford 6-(2-methoxyphenyl)-5H-pyrrolo[2,3-b]pyrazine (600 mg, 40.0%) as a yellow solid. MS m/z: 226 [M+H]⁺.

Step 3: 6-(2-methoxyphenyl)-5-methylpyrrolo[2,3-b]pyrazine: To a stirred solution of 6-(2-methoxyphenyl)-5H-pyrrolo[2,3-b]pyrazine (300 mg, 1.33 mmol, 1 equiv) and Cs₂CO₃ (1302 mg, 4.00 mmol, 3 equiv) in DMF (3 mL) was added MeI (284 mg, 2.00 mmol, 1.5 equiv) at room temperature. The resulting mixture was stirred for 3 h at room temperature. The resulting mixture was extracted with EtOAc (40×mL). The combined organic layers were washed with water (3×20 mL), dried over anhydrous Na₂SO₄. After filtration, the filtrate was concentrated under reduced pressure. This provided 6-(2-methoxyphenyl)-5-methylpyrrolo[2,3-b]pyrazine (300 mg, 94.14%) as a brown solid. MS m/z: 240 [M+H]⁺.

Step 4: 6-(2-methoxyphenyl)-5-methylpyrrolo[2,3-b]pyrazine-7-carbaldehyde: A solution of 6-(2-methoxyphenyl)-5-methylpyrrolo[2,3-b]pyrazine (300 mg, 1.254 mmol, 1 equiv) and POCl₃ (1.5 mL) in DMF (3 mL) was stirred for 12 h at 50° C. Desired product could be detected by LCMS. The resulting mixture was basified to PH ˜8, extracted with EtOAc (3×10 mL). The combined organic layers were washed with water (3×15 mL), dried over anhydrous Na₂SO₄. After filtration, the filtrate was concentrated under reduced pressure. This provided 6-(2-methoxyphenyl)-5-methylpyrrolo[2,3-b]pyrazine-7-carbaldehyde (260 mg, 77.58%) as a brown solid. MS m/z: 268 [M+H]⁺.

Step 5: 6-(2-Methoxyphenyl)-5-methylpyrrolo[2,3-b]pyrazine-7-carboxylic acid: To a stirred solution of 6-(2-methoxyphenyl)-5-methylpyrrolo[2,3-b]pyrazine-7-carbaldehyde (260 mg, 0.973 mmol, 1 equiv) and 2-methyl-2-butene (170.56 mg, 2.433 mmol, 2.5 equiv) in n-BuOH (3 mL)/H₂O (0.5 mL) were added NaH₂PO₄ (700.23 mg, 5.838 mmol, 6 equiv) and NaClO₂ (131.96 mg, 1.460 mmol, 1.5 equiv). The resulting mixture was stirred for 2 h at 50° C. The resulting mixture was purified by reverse flash chromatography with the following conditions: column, C18 silica gel; mobile phase, MeCN in water, 0% to 100% gradient in 30 min; detector, UV 254 nm. This provided 6-(2-methoxyphenyl)-5-methylpyrrolo[2,3-b]pyrazine-7-carboxylic acid (240 mg, 87.1%) as a yellow solid. MS m/z: 284 [M+H]⁺.

Step 6: 1-[6-(2-methoxyphenyl)-5-methylpyrrolo[2,3-b]pyrazine-7-carbonyl]-3-(2-methylphenoxymethyl)piperidine: To a stirred solution of 6-(2-methoxyphenyl)-5-methylpyrrolo[2,3-b]pyrazine-7-carboxylic acid (100 mg, 0.353 mmol, 1.00 equiv) and HATU (147.64 mg, 0.388 mmol, 1.1 equiv) in DMF (1 mL) were added DIEA (136.87 mg, 1.059 mmol, 3 equiv) and 3-(2-methylphenoxymethyl)piperidine hydrochloride (102.41 mg, 0.424 mmol, 1.2 equiv) at 0° C. The resulting mixture was stirred for 2 h at room temperature. Desired product could be detected by LCMS. The resulting mixture was purified by reverse flash chromatography with the following conditions: column, silica gel; mobile phase, MeCN in water, 0% to 100% gradient in 30 min; detector, UV 254 nm. This provided 1-[6-(2-methoxyphenyl)-5-methylpyrrolo[2,3-b]pyrazine-7-carbonyl]-3-(2-methylphenoxymethyl)piperidine (36 mg, 21.7%) as a light yellow solid. ¹H NMR (400 MHz, DMSO-d₆) δ 8.53-8.20 (m, 2H), 7.55-7.53 (m, 1H), 7.40-7.16 (m, 2H), 7.19-7.01 (m, 2H), 7.01-6.66 (m, 3H), 4.33 (s, 1H), 3.80-3.71 (m, 5H), 3.59 (s, 4H), 2.98-2.78 (m, 2H), 2.16 (s, 1H), 1.78 (s, 3H), 1.45-1.24 (m, 4H). MS m/z: 471.2 [M+H]⁺.

### (6-cyclopropyl-5-phenyl-5H-pyrrolo[2,3-b]pyrazin-7-yl)(3-((o-tolyloxy)methyl)piperidin-1-yl)methanone (255)

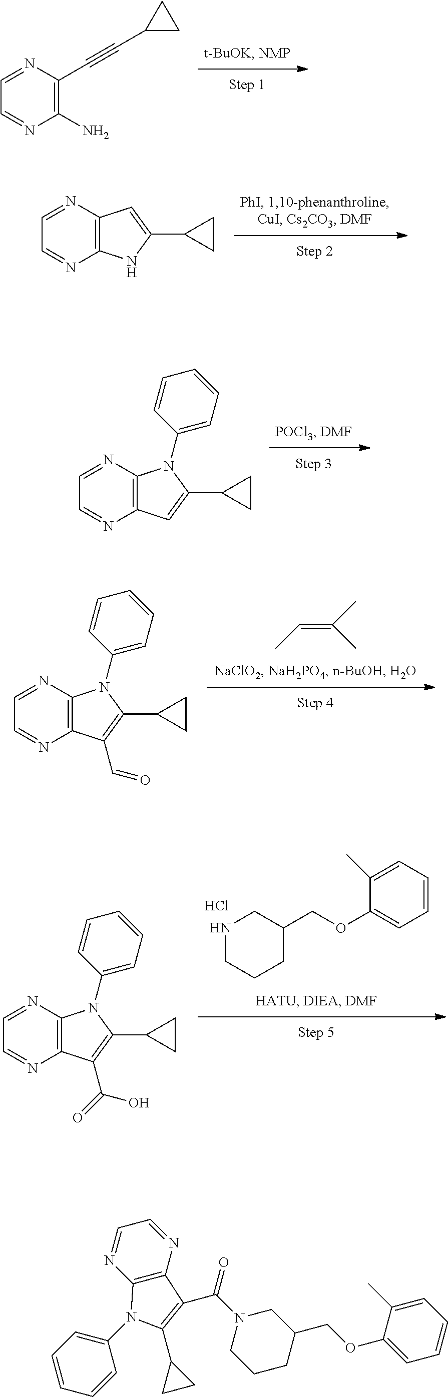

Step 1: 6-cyclopropyl-5H-pyrrolo[2,3-b]pyrazine: A solution of 3-(cyclopropylethynyl)pyrazin-2-amine (1.00 g. 6.28 mmol, 1.00 equiv) and t-BuOK (1.06 g, 9.42 mmol, 1.50 equiv) in NMP (10.0 mL) was stirred for 10 h at 80° C. The resulting mixture was diluted with EtOAc (50 mL). The organic layers were washed with water (3×30 mL), dried over anhydrous Na₂SO₄. After filtration, the filtrate was concentrated under reduced pressure. The residue was purified by silica gel column chromatography, eluted with EtOAc/PE (1/2) to afford 6-cyclopropyl-5H-pyrrolo[2,3-b]pyrazine (700 mg, 70.0%) as a yellow solid. MS m/z: 160 [M+H]⁺.

Step 2: 6-cyclopropyl-5-phenyl-5H-pyrrolo[2,3-b]pyrazine: To a stirred solution of 6-cyclopropyl-5H-pyrrolo[2,3-b]pyrazine (300 mg, 1.89 mmol, 1.00 equiv) and iodobenzene (769 mg, 3.77 mmol, 2.00 equiv) in DMF (3.00 mL) were added CuI (35.9 mg, 0.189 mmol, 0.100 equiv) and 1,10-phenanthroline (67.9 mg, 0.377 mmol, 0.200 equiv) and Cs₂CO₃ (1842 mg. 5.66 mmol, 3.00 equiv). The resulting mixture was stirred for 12 h at 120° C. under nitrogen atmosphere. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with EtOAc/PE (1/1) to afford 6-cyclopropyl-5-phenyl-5H-pyrrolo[2,3-b]pyrazine (320 mg, 72.2%) as a yellow solid. MS m/z: 236 [M+H]⁺.

Step 3: 6-cyclopropyl-5-phenyl-5H-pyrrolo[2,3-b]pyrazine-7-carbaldehyde: A solution of 6-cyclopropyl-5-phenyl-5H-pyrrolo[2,3-b]pyrazine (320 mg, 1.36 mmol, 1.00 equiv) and POCl₃ (626 mg, 4.07 mmol, 3.00 equiv) in DMF (4.00 mL) was stirred for overnight at 0° C. under air atmosphere. The reaction was quenched with water at room temperature. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with EtOAc/PE (1/1) to afford 6-cyclopropyl-5-phenyl-5H-pyrrolo[2,3-b]pyrazine-7-carbaldehyde (200 mg, 60.0%) as a white solid. MS m/z: 264 [M+H]⁺.

Step 4: 6-cyclopropyl-5-phenyl-5H-pyrrolo[2,3-b]pyrazine-7-carboxylic acid: To a stirred solution of 6-cyclopropyl-5-phenyl-5H-pyrrolo[2,3-b]pyrazine-7-carbaldehyde (200 mg, 0.760 mmol, 1.00 equiv) and 2-methylbut-2-ene (133 mg, 1.90 mmol, 2.50 equiv) in n-BuOH (2.00 mL)/H₂O (0.500 mL) were added NaH₂PO₄ (547 mg, 4.56 mmol, 6.00 equiv) and NaClO₂ (103 mg, 1.14 mmol, 1.50 equiv). The resulting mixture was stirred for 2 h at 50° C. The resulting mixture was diluted with EtOAc (15 mL). The organic layers were washed with water (3×10 mL), dried over anhydrous Na₂SO₄. After filtration, the filtrate was concentrated under reduced pressure. The residue was purified by reverse flash chromatography with the following conditions: column, silica gel; mobile phase, MeCN in water, 0% to 100% gradient in 15 min; detector, UV 254 nm. This provided 6-cyclopropyl-5-phenyl-5H-pyrrolo[2,3-b]pyrazine-7-carboxylic acid (180 mg, 84.8%) as a yellow solid. MS m/z: 280 [M+H]⁺.

Step 5: (6-cyclopropyl-5-phenyl-5H-pyrrolo[2,3-b]pyrazin-7-yl)(3-((o-tolyloxy)meth-yl)pi-peridin-1-yl)methanone: To a stirred solution of 6-cyclopropyl-5-phenyl-5H-pyrrolo[2,3-b]pyrazine-7-carboxylic acid (100 mg, 0.358 mmol, 1.00 equiv) and HATU (150 mg, 0.394 mmol, 1.10 equiv) in DMF (1.00 mL) were added DIPEA (139 mg, 1.07 mmol, 3.00 equiv) and 3-((o-tolyloxy)methyl)piperidine hydrochloride (104 mg, 0.430 mmol, 1.20 equiv) at 0° C. The resulting mixture was stirred for 3 h at room temperature. Desired product could be detected by LCMS. The resulting mixture was diluted with EtOAc (15 mL). The organic layer was washed with water (3×10 mL), dried over anhydrous Na₂SO₄. After filtration, the filtrate was concentrated under reduced pressure. The residue was purified by reverse flash chromatography with the following conditions: column, silica gel; mobile phase, MeCN in water, 0% to 100% gradient in 30 min; detector, UV 254 nm. This provided (6-cyclopropyl-5-phenyl-5H-pyrrolo[2,3-b]pyrazin-7-yl)(3-((o-tolyloxy)methyl)piperidin-1-yl)methanone (50.0 mg, 29.9%) as a light yellow solid. ¹H NMR (400 MHZ, DMSO-d₆) δ 8.39 (dd, J=28.3, 2.6 H₂, 1H), 8.19 (dd, J=11.4, 2.6 Hz, 1H), 7.64-7.50 (m, 5H), 7.17-6.73 (m, 4H), 4.54 (dd, J=50.0, 12.7 Hz, 1H), 4.01-3.80 (m, 2H), 3.58 (t, J=9.3 Hz, 1H), 3.12-2.93 (m, 2H), 2.22 (s, 1H), 2.12-2.00 (m, 1H). 1.93-1.79 (m, 3H). 1.63-1.38 (m, 4H), 0.81 (d, J=7.9 Hz, 4H). MS m/z: 467 [M+H]⁺.

### (5-Isopropyl-6-phenyl-5H-pyrrolo[2,3-b]pyrazin-7-yl)(3-((o-tolyloxy)methyl)piperidin-1-yl)methanone (256)

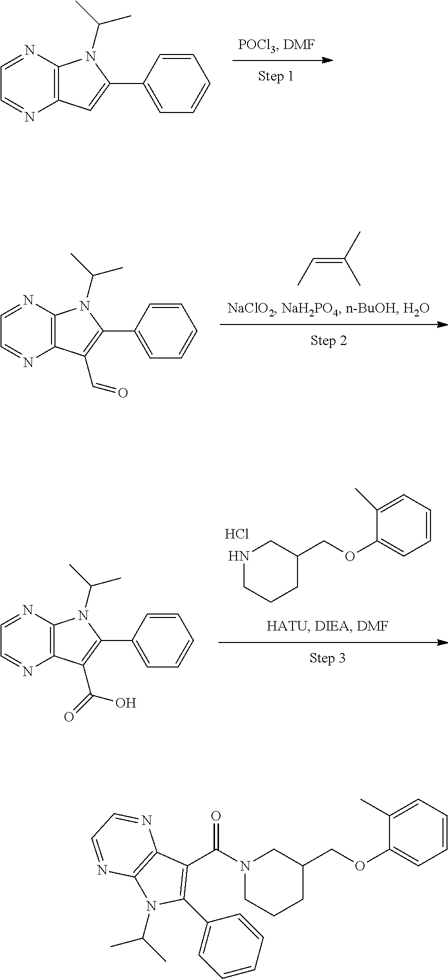

Step 1: 5-isopropyl-6-phenyl-5H-pyrrolo[2,3-b]pyrazine-7-carbaldehyde: A solution of 5-isopropyl-6-phenyl-5H-pyrrolo[2,3-b]pyrazine (100 mg, 0.421 mmol, 1.00 equiv) and POCl₃ (0.500 mL, 10.7 mmol, 25.5 equiv) in DMF (1.00 mL) was stirred for 12 h at 50° C. The reaction was quenched with water at room temperature. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by silica gel column chromatography, eluted with EtOAc/PE (1/1) to afford 5-isopropyl-6-phenyl-5H-pyrrolo[2,3-b]pyrazine-7-carbaldehyde (90.0 mg, 80.5%) as a yellow solid. MS m/z: 266 [M+H]⁺.

Step 2: 5-isopropyl-6-phenyl-5H-pyrrolo[2,3-b]pyrazine-7-carboxylic acid: To a stirred solution of 5-isopropyl-6-phenyl-5H-pyrrolo[2,3-b]pyrazine-7-carbaldehyde (90.00 mg. 0.339 mmol, 1.00 equiv) and 2-methylbut-2-ene (59.5 mg, 0.848 mmol, 2.500 equiv) in n-BuOH (1.00 mL)/H₂O (0.200 mL) were added NaH₂PO₄ (244 mg, 2.03 mmol, 6.00 equiv) and NaClO₂ (46.0 mg, 0.509 mmol, 1.50 equiv). The resulting mixture was stirred for 2 h at 50° C. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by reverse flash chromatography with the following conditions: column, silica gel; mobile phase, MeCN in water, 0% to 100% gradient in 15 min; detector, UV 254 nm. This provided 5-isopropyl-6-phenyl-5H-pyrrolo[2,3-b]pyrazine-7-carboxylic acid (90.0 mg, 94.3%) as a yellow solid. MS m/z: 282 [M+H]⁺.

Step 3: (5-isopropyl-6-phenyl-5H-pyrrolo[2,3-b]pyrazin-7-yl)(3-((o-tolyloxy) methyl)piperidin-1-yl)methanone: To a stirred solution of 5-isopropyl-6-phenyl-5H-pyrrolo[2,3-b]pyrazine-7-carboxylic acid (90.0 mg, 0.320 mmol, 1.00 equiv) and HATU (134 mg, 0.352 mmol, 1.100 equiv) in DMF (1.00 mL) were added DIPEA (124 mg, 0.960 mmol, 3.00 equiv) and 3-((o-tolyloxy)methyl)piperidine hydrochloride (92.8 mg, 0.384 mmol, 1.20 equiv) at 0° C. The resulting mixture was stirred for 3 h at room temperature. Desired product could be detected by LCMS. The resulting mixture was diluted with EtOAc (15 mL). The organic layer was washed with water (3×10 mL), dried over anhydrous Na₂SO₄. After filtration, the filtrate was concentrated under reduced pressure. The residue was purified by reverse flash chromatography with the following conditions: column, silica gel; mobile phase, MeCN in water, 0% to 100% gradient in 30 min; detector, UV 254 nm. This provided (5-isopropyl-6-phenyl-5H-pyrrolo[2,3-b]pyrazin-7-yl)(3-((o-tolyloxy)methyl)piperidin-1-yl)methanone (40.0 mg, 26.7%) as a light yellow solid. ¹H NMR (400 MHZ, DMSO-d₆) δ 8.49-8.39 (m, 1H), 8.35 (d, J=11.5 Hz, 1H), 7.47 (d, J=48.9 Hz, 5H), 7.18-6.93 (m, 2H), 6.92-6.79 (m, 1H), 6.75 (d, J=7.5 Hz, 1H), 4.48 (t, J=6.7 Hz, 1H), 4.32 (t, J=15.8 Hz, 1H), 3.83 (s, 2H), 3.50 (t, J=9.5 Hz, 1H), 2.98-2.81 (m, 2H), 2.20-2.12 (m, 1H), 1.84-1.57 (m, 9H), 1.43 (s, 4H). MS m/z: 469 [M+H]⁺.

### (3,5-Dimethyl-6-phenyl-5H-pyrrolo[2,3-b]pyrazin-7-yl)(3-((o-tolyloxy)methyl)piperidin-1-yl)methanone (257)

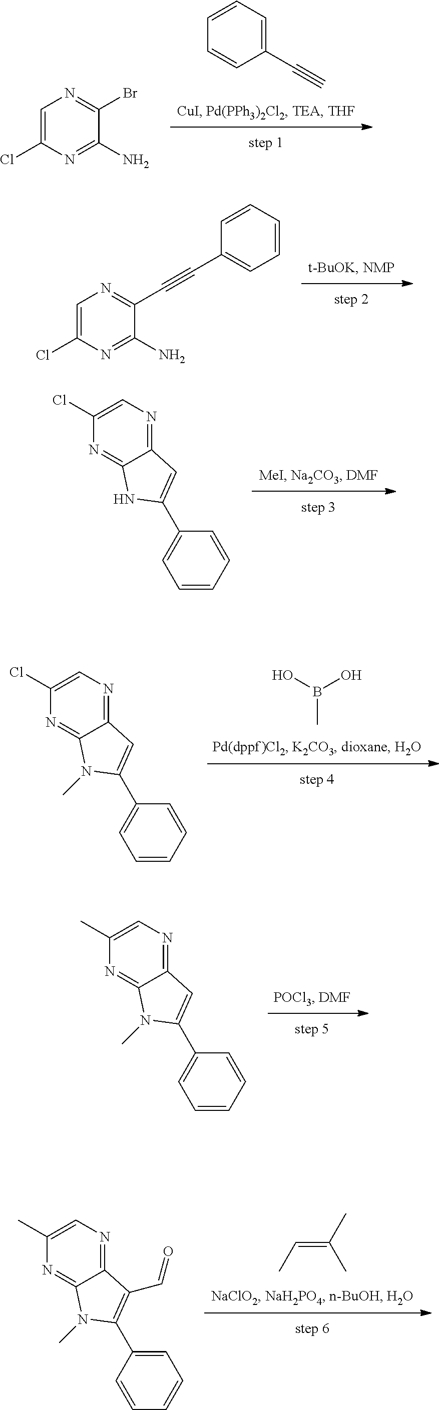

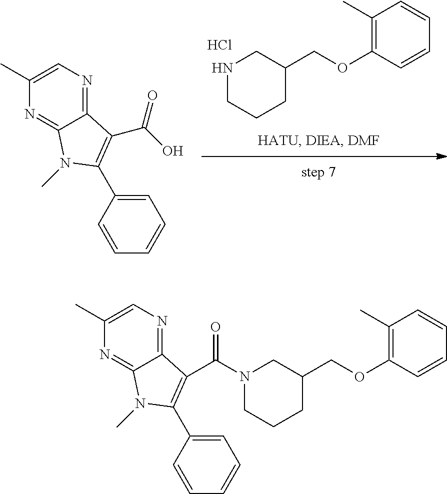 [SMILES: Cc1cnc2c(C(=O)O)c(-c3ccccc3)n(C)c2n1 ; Cl ; Cc1ccccc1OCC1CCCNC1 ; Cc1cnc2c(C(=O)N3CCCC(COc4ccccc4C)C3)c(-c3ccccc3)n(C)c2n1]

Step 1: 6-chloro-3-(phenylethynyl)pyrazin-2-amine: A mixture of 3-bromo-6-chloropyrazin-2-amine (1000 mg, 4.83 mmol, 1 equiv) and phenylacetylene (985 mg, 9.66 mmol, 2 equiv), CuI (92.2 mg, 0.483 mmol, 0.1 equiv), Pd(PPh₃)₂Cl₂ (353 mg, 0.483 mmol, 0.1 equiv), TEA (1.46 g, 20.9 mmol, 3 equiv) in THF (10 mL) was stirred for 2 h at 80° C. under inert atmosphere. The mixture was allowed to cool down to room temperature and concentrated. The residue was purified by flash chromatography, eluted with PE/EtOAc (1/1). This provided 5-methyl-3-(2-phenylethynyl)pyrazin-2-amine (1.00 g, 90.4%) as a white solid. MS m/z: 230 [M+H]⁺.

Step 2: 3-chloro-6-phenyl-5H-pyrrolo[2,3-b]pyrazine: A mixture of 5-methyl-3-(2-phenylethynyl)pyrazin-2-amine (1.00 g. 3.94 mmol, 1 equiv) and t-BuOK (531 mg, 4.73 mmol, 1.2 equiv) in NMP (10 mL) was stirred for 2 h at 100° C. under inert atmosphere. The mixture was allowed to cool down to room temperature. The reaction was monitored by LCMS. Desired product could be detected by LCMS. The residue was purified by reverse flash chromatography with the following conditions: column, C18 silica gel, mobile phase, MeCN in Water (0.1% FA), 5% to 100% gradient in 20 min, detector, UV 254 nm. This provided 2-methyl-6-phenyl-5H-pyrrolo[2,3-b]pyrazine (700 mg, 70.0%) as a white solid. MS m/z: 230 [M+H]⁺.

Step 3: 3-chloro-5-methyl-6-phenyl-5H-pyrrolo[2,3-b]pyrazine: A mixture of 2-methyl-6-phenyl-5H-pyrrolo[2,3-b]pyrazine (700 mg, 3.06 mmol, 1 equiv), MeI (434 mg, 3.06 mmol, 1 equiv) and Cs₂CO₃ (2.00 g, 6.12 mmol, 2 equiv) in DMF (7 mL) was stirred for 16 h at room temperature under air atmosphere. The resulting mixture was concentrated and purified by reverse flash chromatography with the following conditions: column, C18 silica gel, mobile phase, MeCN in Water (0.1% FA), 5% to 100% gradient in 20 min, detector, UV 254 nm. This provided 3-chloro-5-methyl-6-phenyl-5H-pyrrolo[2,3-b]pyrazine (600 mg, 80.6%) as a white solid. MS m/z: 244 [M+H]⁺.

Step 4: 3,5-dimethyl-6-phenyl-5H-pyrrolo[2,3-b]pyrazine: To the solution of 3-chloro-5-methyl-6-phenyl-5H-pyrrolo[2,3-b]pyrazine (600 mg, 2.46 mmol, 1 equiv) and methylboronic acid (592 mg, 9.88 mmol, 4 equiv) in dioxane (6 mL) were added Pd(dppf)Cl₂ (89.5 mg, 0.123 mmol, 0.05 equiv) and Na₂CO₃ (782 mg, 7.38 mmol, 3 equiv) under N₂ atmosphere. The result mixture was heated to 80° C. and stirred overnight. Desired product could be detected by LCMS. The reaction mixture was diluted by EtOAc (50 mL), washed by water (2×40 mL) and brine (1×40 mL), dried over anhydrous sodium sulfate. After filtration, the filtrate was concentrated. The residue was purified by silica gel column chromatography, eluted with EtOAc/PE=1/1, to afford 3,5-dimethyl-6-phenyl-5H-pyrrolo[2,3-b]pyrazine (420 mg, 76.5%) as colorless oil. MS m/z: 224 [M+H]⁺.

Step 5: 3,5-dimethyl-6-phenyl-5H-pyrrolo[2,3-b]pyrazine-7-carbaldehyde: To a stirred solution of POCl₃ (411 mg, 2.69 mmol, 1.5 equiv) in DMF (5 mL) was added 3,5-dimethyl-6-phenyl-5H-pyrrolo[2,3-b]pyrazine (400 mg, 1.79 mmol, 1 equiv) in portions at 0° C. The resulting mixture was stirred for 2 h at 0° C. under nitrogen atmosphere. The reaction was monitored by LCMS. The mixture was diluted by water (20 mL) neutralized to pH ˜8 with saturated NaHCO₃(aq.). The resulting mixture was extracted with EtOAc (3×10 mL). The combined organic layers were washed with water (2×20 mL) and brine (20 mL), dried over anhydrous Na₂SO₄. After filtration, the filtrate was concentrated under reduced pressure. This provided 3,5-dimethyl-6-phenyl-5H-pyrrolo[2,3-b]pyrazine-7-carbaldehyde (250 mg, 55.6%) as a yellow solid. MS m/z: 252 [M+H]⁺.

Step 6: 3,5-dimethyl-6-phenyl-5H-pyrrolo[2,3-b]pyrazine-7-carboxylic acid: To a stirred solution of 3,5-dimethyl-6-phenyl-5H-pyrrolo[2,3-b]pyrazine-7-carbaldehyde (250 mg, 1.00 mmol, 1.00 equiv) in H₂O (1.00 mL) was added NaH₂PO₄ (720 mg, 6.00 mmol, 6.00 equiv) at 0° C. under air atmosphere. After 5 minutes, t-BuOH (5.00 mL), 2,3-dimethylbut-2-ene (208 mg, 2.5 mmol, 2.50 equiv) and NaClO₂ (167 mg, 1.50 mmol, 1.50 equiv) were added. After 16 hours, reaction mixture was diluted with ethyl acetate (20 mL), washed with water (2×15 mL) and brine (20 mL), then dried over magnesium sulfate, filtered, and concentrated to give 3,5-dimethyl-6-phenyl-5H-pyrrolo[2,3-b]pyrazine-7-carboxylic acid (150 mg, 56.0%) as a white solid which was used without further purification. MS m/z: 268 [M+H]⁺.

Step 7: (3,5-dimethyl-6-phenyl-5H-pyrrolo[2,3-b]pyrazin-7-yl)(3-((o-tolyloxy)methyl)piperidin-1-yl)methanone: To a stirred mixture of 3-((o-tolyloxy)methyl)piperidine hydrochloride (56.7 mg, 0.449 mmol, 1.2 equiv) and HATU (85 mg. 0.225 mmol, 1.2 equiv) in DMF (1 mL) were added DIEA (72.5 mg, 6.06 mmol, 3 equiv) and 3,5-dimethyl-6-phenyl-5H-pyrrolo[2,3-b]pyrazine-7-carboxylic acid (50 mg, 0.187 mmol, 1.00 equiv) in portions at 0° C. under inert atmosphere. The resulting mixture was stirred for 2 h at room temperature under inert atmosphere. The resulting mixture was purified by reverse flash chromatography with the following conditions: column, C18 silica gel, mobile phase, ACN in water, 5% to 100% gradient in 20 min, detector, UV 254 nm. This provided (3,5-dimethyl-6-phenyl-5H-pyrrolo[2,3-b]pyrazin-7-yl)(3-((o-tolyloxy)methyl)piperidin-1-yl)methanone (30 mg, 35.2%) as a white solid. ¹H NMR (400 MHz, DMSO-d₆) δ 8.34 (d, J=47.1 Hz, 1H), 7.71-7.36 (m, 5H), 7.22-6.67 (m, 4H), 4.51-4.26 (m, 1H), 3.97-3.61 (m, 5H), 3.61-3.42 (m, 1H), 3.00-2.73 (m, 2H), 2.67-2.58 (m, 3H), 2.17 (s, 1H), 1.99-1.60 (m, 3H), 1.47 (s, 2H), 1.43-1.20 (m, 2H). MS m/z: 455.20 [M+H]⁺.

### (2-Benzylmorpholino)(3-(((3-(trifluoromethyl)pyridin-2-yl)oxy)methyl)piperidin-1-yl)methanone (258)

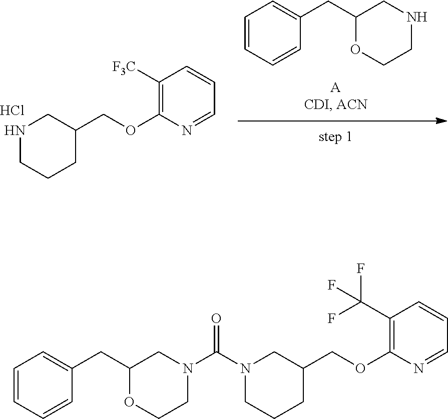

To a stirred mixture of 2-benzylmorpholine (50.0 mg, 0.282 mmol, 1.00 equiv) and 2-(piperidin-3-ylmethoxy)-3-(trifluoromethyl) pyridine hydrochloride (73.4 mg, 0.282 mmol, 1.00 equiv) in ACN (1.50 mL) was added CDI (45.7 mg, 0.282 mmol, 1.00 equiv) in portions at room temperature under air atmosphere. The resulting mixture was stirred for 1 h at room temperature under air atmosphere. The resulting mixture was concentrated under reduced pressure. The residue was purified by silica gel column chromatography, eluted with PE/EA (0% to 100% gradient in 20 min) to afford 2-benzyl-4-[3-({[3-(trifluoromethyl)pyridin-2-yl]oxy}methyl) piperidine-1-carbonyl]morpholine (crude). The crude product was purified by Prep-HPLC with the following conditions (Column: Xselect CSH F-Phenyl OBD column, 19*250 mm, 5 um; Mobile Phase A: Water(0.1% FA), Mobile Phase B: ACN; Flow rate: 25 mL/min; Gradient: 47% B to 63% B in 9 min, 63% B; Wave Length: 254/220 nm; RT1(min): 8.47; Number Of Runs: 0) to afford (2-benzylmorpholino)(3-(((3-(trifluoromethyl)pyridin-2-yl)oxy)methyl)piperidin-1-yl)methanone (10.9 mg, 7.8%) as a yellow oil. ¹H NMR (400 MHz, CDCl₃) δ 8.27-8.26 (m, 1H), 7.47-7.43 (m, 1H), 7.34-7.32 (m, 1H), 7.31-7.28 (m, 1H), 7.25 (s, 1H), 7.22-7.17 (m, 3H), 3.93-3.79 (m, 3H), 3.73-3.53 (m, 4H), 3.51-3.40 (m, 2H), 3.08-2.96 (m, 1H), 2.93-2.86 (m, 1H), 2.81-2.64 (m, 4H), 2.02-1.95 (m, 1H), 1.90-1.81 (m, 1H), 1.50-1.25 (m, 3H). MS m/z: 464.1 [M+H]⁺

### 6-[(1R,5S,6R)-6-({[2-(Trifluoromethyl)pyridin-3-yl]oxy}methyl)-3-azabicyclo[3.1.0]hexane-3-carbonyl]-1H-indole (259)

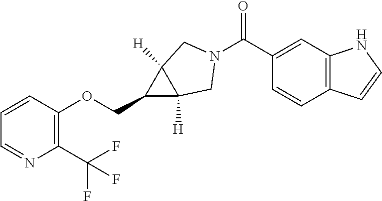 [SMILES: O=C(c1ccc2cc[nH]c2c1)N1C[C@@H]2[C@H](COc3cccnc3C(F)(F)F)[C@@H]2C1]

Followed General Procedure E using 1H-indole-6-carboxylic acid (16.4 mg, 0.1 mmol) and (1R,5S,6R)-6-({[2-(trifluoromethyl)pyridin-3-yl]oxy}methyl)-3-azabicyclo[3.1.0]hexane hydrochloride (30 mg, 0.1 mmol) to afford 6-[(1R,5S,6R)-6-({[2-(trifluoromethyl)pyridin-3-yl]oxy}methyl)-3-azabicyclo[3.1.0]hexane-3-carbonyl]-1H-indole (15 mg, 37%) as a colorless oil. ¹H NMR (500 MHZ, DMSO-d₆) δ 11.19 (s, 1H), 8.26 (dd, J=4.5, 1.2 Hz, 1H), 7.79 (dd, J=8.6, 1.1 Hz, 1H), 7.71 (dd, J=8.6, 4.5 Hz, 1H), 7.43 (t, J=2.7 Hz, 1H), 7.41-7.32 (m, 2H), 6.84 (dd, J=8.2, 1.5 Hz, 1H), 6.43 (ddd, J=3.0, 1.9, 0.9 Hz, 1H), 4.23 (t, J=9.6 Hz, 1H), 4.07 (t, J=9.4 Hz, 1H), 3.94 (d, J=12.7 Hz, 1H), 3.82 (d, J=12.5 Hz, 1H), 3.64 (dd, J=12.4, 4.7 Hz, 1H), 3.52 (d, J=11.1 Hz, 1H), 1.94-1.83 (m, 2H), 1.46 (p. J=8.0 Hz, 1H). MS m/z: 402.2 [M+H]⁺

### 3-[1-(2,2-Difluoroethyl)-1H-pyrazolo[3,4-b]pyrazin-6-yl]-1-({[2-(trifluoromethyl)pyridin-3-yl]oxy}methyl)-3-azabicyclo[3.1.0]hexane (260)

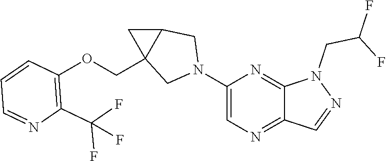 [SMILES: FC(F)Cn1ncc2ncc(N3CC4CC4(COc4cccnc4C(F)(F)F)C3)nc21]

Step 1: tert-butyl 1-({[2-(trifluoromethyl)pyridin-3-yl]oxy}methyl)-3-azabicyclo[3.1.0]hexane-3-carboxylate: Followed General Procedure A using tert-butyl 1-(hydroxymethyl)-3-azabicyclo[3.1.0]hexane-3-carboxylate (50 mg, 0.23 mmol, 1.00 equiv) and 2-(trifluoromethyl)pyridin-3-ol (38 mg, 0.23 mmol, 1.0 equiv) to afford tert-butyl 1-({[2-(trifluoromethyl)pyridin-3-yl]oxy}methyl)-3-azabicyclo[3.1.0]hexane-3-carboxylate (110 mg, 130%, crude) as a colorless oil. MS m/z: 359.2 [M+H]⁺.

Step 2: 1-({[2-(trifluoromethyl)pyridin-3-yl]oxy}methyl)-3-azabicyclo[3.1.0]hexane hydrochloride: Followed General Procedure B using tert-butyl 1-({[2-(trifluoromethyl)pyridin-3-yl]oxy}methyl)-3-azabicyclo[3.1.0]hexane-3-carboxylate (110 mg, 1 mmol, 1.00 equiv) to afford the crude product 1-({[2-(trifluoromethyl)pyridin-3-yl]oxy}methyl)-3-azabicyclo[3.1.0]hexane hydrochloride (100 mg). MS m/z: 259 [M+H]⁺.

Step 3: 3-[1-(2,2-difluoroethyl)-1H-pyrazolo[3,4-b]pyrazin-6-yl]-1-({[2-(trifluoromethyl)pyridin-3-yl]oxy}methyl)-3-azabicyclo[3.1.0]hexane: Followed General Procedure C using 2 1-({[2-(trifluoromethyl)pyridin-3-yl]oxy}methyl)-3-azabicyclo[3.1.0]hexane (22.3 mg, 0.1 mmol, 1.00 equiv) and 6-chloro-1-(2,2-difluoroethyl)-1H-pyrazolo[3,4-b]pyrazine (30 mg, 0.1 mmol, 1.00 equiv) to afford 3-[1-(2,2-difluoroethyl)-1H-pyrazolo[3,4-b]pyrazin-6-yl]-1-({[2-(trifluoromethyl)pyridin-3-yl]oxy}methyl)-3-azabicyclo[3.1.0]hexane (11 mg, 25%) as a colorless oil. ¹H NMR (500 MHZ, DMSO-d₆) δ 8.28 (dd, J=4.5, 1.2 Hz, 1H), 8.14 (s, 1H), 8.08 (s, 1H), 7.81 (dd, J=8.8, 1.2 Hz, 1H), 7.71 (dd, J=8.6, 4.5 Hz, 1H), 6.44 (t, J=3.8 Hz, 1H), 4.67 (td, J=15.0, 3.9 Hz, 2H), 4.47-4.38 (m, 2H), 4.00 (d, J=10.7 Hz, 1H), 3.91 (d, J=10.7 Hz, 1H), 3.64 (dd, J=14.8, 9.1 Hz, 2H), 1.85 (dt, J=8.5, 4.3 Hz, 1H), 1.07 (dd, J=8.2, 4.9 Hz, 1H), 0.65 (t, J=4.6 Hz, 1H). MS m/z: 441.2 [M+H]⁺.

### 6-[1-({[2-(Trifluoromethyl)pyridin-3-yl]oxy}methyl)-3-azabicyclo[3.1.0]hexane-3-carbonyl]-1H-indole (261)

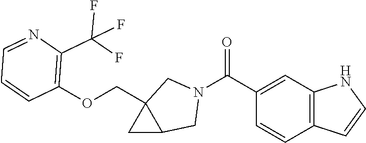 [SMILES: O=C(c1ccc2cc[nH]c2c1)N1CC2CC2(COc2cccnc2C(F)(F)F)C1]

Followed General Procedure E using 1H-indole-6-carboxylic acid (16.4 mg, 0.1 mmol) and 1-({[2-(trifluoromethyl)pyridin-3-yl]oxy}methyl)-3-azabicyclo[3.1.0]hexane hydrochloride (30 mg, 0.1 mmol) to afford 6-[1-({[2-(trifluoromethyl)pyridin-3-yl]oxy}methyl)-3-azabicyclo[3.1.0]hexane-3-carbonyl]-1H-indole (9 mg, 22%) as a colorless oil. ¹H NMR (500 MHZ, DMSO-d₆) δ 11.28 (s, 1H), 8.25 (d, J=15.6 Hz, 1H), 7.72 (t, J=27.9 Hz, 2H), 7.56 (d, J=8.2 Hz, 1H), 7.52-7.49 (m, 1H), 7.46 (t, J=2.8 Hz, 1H), 7.09 (dd, J=8.1, 1.5 Hz, 1H), 6.47 (ddd, J=3.0, 1.9, 0.9 Hz, 1H), 4.49-4.24 (m, 2H), 4.12 (dd, J=62.7, 11.5 Hz, 1H), 3.81-3.70 (m, 1H), 3.64-3.40 (m, 2H), 1.63 (d, J=19.7 Hz, 1H), 0.93 (dd, J=8.1, 5.0 Hz, 1H), 0.50 (t, J=4.6 Hz, 1H). MS m/z: 402.2 [M+H]⁺.

### 2-(6-((3S,5R)-3-Methyl-5-(((2-(trifluoromethyl)pyridin-3-yl)oxy)methyl)piperidin-1-yl)pyrazin-2-yl)-1,3,4-thiadiazole (262a) and 2-(6-((3S,5S)-3-methyl-5-(((2-(trifluoromethyl)pyridin-3-yl)oxy)methyl)piperidin-1-yl)pyrazin-2-yl)-1,3,4-thiadiazole (262b)

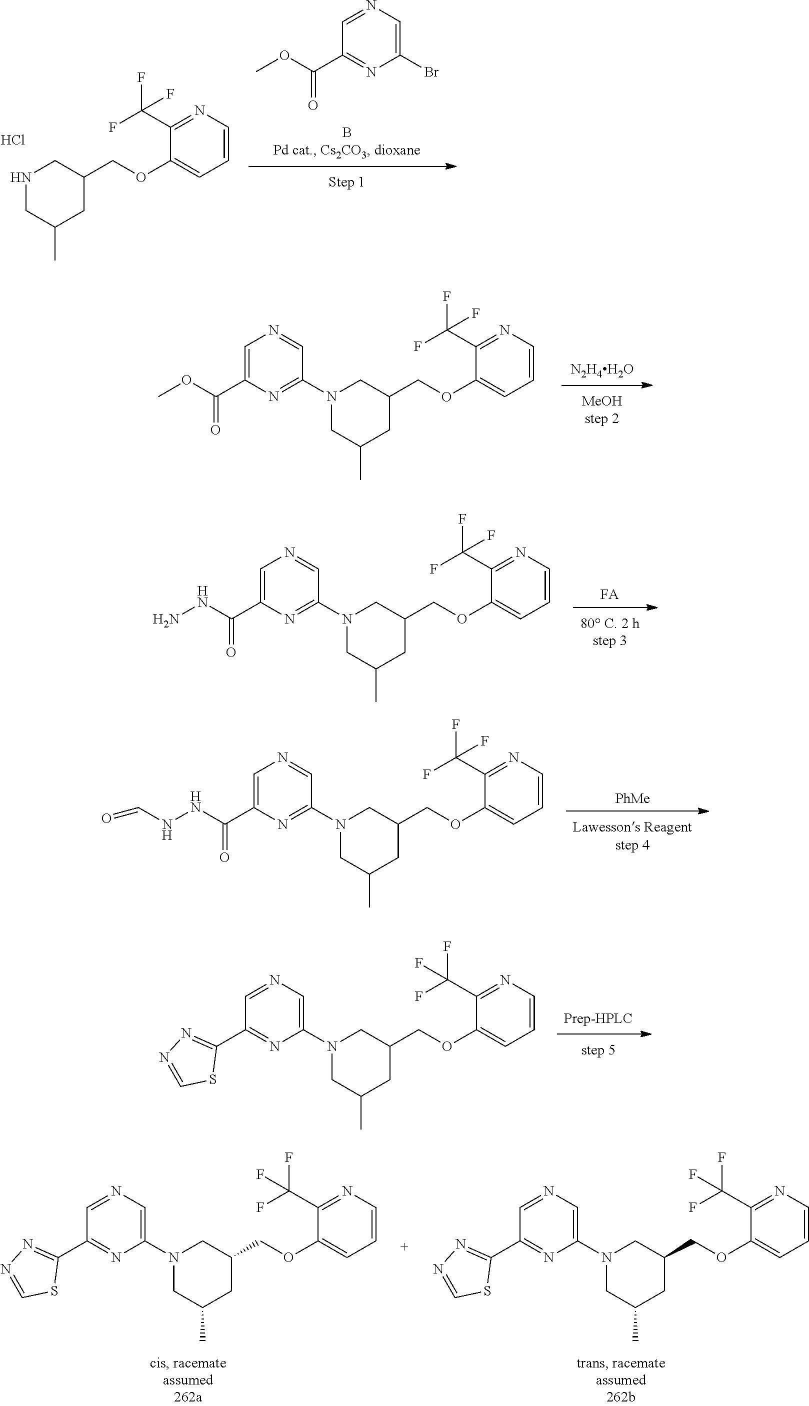

Step 1: methyl 6-(3-methyl-5-(((2-(trifluoromethyl)pyridin-3-yl)oxy)methyl)piperidin-1-yl)pyrazine-2-carboxylate: A solution of methyl 6-bromopyrazine-2-carboxylate (132 mg, 0.611 mmol, 1.0 equiv) and 1612891-29-8 (51.4 mg, 0.061 mmol, 0.1 equiv) and Cs₂CO₃ (597 mg, 1.83 mmol, 3 equiv) in dioxane (2 mL) was added 3-((5-methylpiperidin-3-yl)methoxy)-2-(trifluoromethyl)pyridine hydrochloride (190 mg, 0.611 mmol, 1 equiv). The resulting mixture was stirred for 2 hours at 100 C. The resulting mixture was diluted with EA (20 mL), washed by water (2×20 mL) and brine (20 mL), dried over anhydrous Na₂SO₄. After filtration, the filtrate was concentrated under reduced pressure. The residue was purified by reverse flash chromatography with the following conditions: column, silica gel; mobile phase, MeCN in water, 5% to 100% gradient in 15 min; detector, UV 254 nm. This provided methyl 6-(3-methyl-5-(((2-(trifluoromethyl)pyridin-3-yl)oxy)methyl)piperidin-1-yl)pyrazine-2-carboxylate (60 mg, 23.9%) as a yellow solid. MS m/z: 411 [M+H]⁺.

Step 2: 6-(3-methyl-5-(((2-(trifluoromethyl)pyridin-3-yl)oxy)methyl)piperidin-1-yl)pyrazine-2-carbohydrazide: To a stirred solution of methyl 6-(3-methyl-5-(((2-(trifluoromethyl)pyridin-3-yl)oxy)methyl)piperidin-1-yl)pyrazine-2-carboxylate (60 mg, 0.146 mmol, 1 equiv) in MeOH (1 mL) was added hydrazine hydrate (98%, 1 mL) dropwise at 0° C. The resulting mixture was stirred for 2 hours at 80° C. The resulting mixture was concentrated under reduced pressure. The residue was purified by reverse flash chromatography with the following conditions: column, silica gel; mobile phase, MeCN in water, 5% to 100% gradient in 15 min; detector, UV 254 nm. This provided 6-(3-methyl-5-(((2-(trifluoromethyl)pyridin-3-yl)oxy)methyl)piperidin-1-yl)pyrazine-2-carbohydrazide (40 mg, 66.7%) as a yellow semi-solid. MS m/z: 411 [M+H]⁺.

Step 3: N′-formyl-6-(3-methyl-5-(((2-(trifluoromethyl)pyridin-3-yl)oxy)methyl)piperidin-1-yl)pyrazine-2-carbohydrazide: To a stirred solution of 6-(3-methyl-5-(((2-(trifluoromethyl)pyridin-3-yl)oxy)methyl)piperidin-1-yl)pyrazine-2-carbohydrazide (40 mg, 0.097 mmol, 1 equiv) in formic acid (1 mL). The resulting mixture was stirred for 2 hours at 80° C. The resulting mixture was concentrated under reduced pressure. The residue was purified by reverse flash chromatography with the following conditions: column, C 18 silica gel; mobile phase, MeCN in water, 5% to 100% gradient in 15 min; detector, UV 254 nm. This resulted N′-formyl-6-(3-methyl-5-(((2-(trifluoromethyl)pyridin-3-yl)oxy)methyl)piperidin-1-yl)pyrazine-2-carbohydrazide (28 mg, 63.2%) as a yellow oil. MS m/z: 439[M+H]⁺.

Step 4: 2-(6-(3-methyl-5-(((2-(trifluoromethyl)pyridin-3-yl)oxy)methyl)piperidin-1-yl)pyrazin-2-yl)-1,3,4-thiadiazole: To a stirred solution of N′-formyl-5-[3-methyl-5-({[2-(trifluoromethyl)pyridin-3-yl]oxy}methyl)piperidin-1-yl]pyrazine-2-carbohydrazide (28 mg, 0.064 mmol, 1 equiv) in methylbenzene (1 mL) was added Lawesson's Reagent(15.5 mg, 0.038 mmol, 0.6 equiv). The resulting mixture was stirred for 2 hours at 80° C. The resulting mixture was concentrated under reduced pressure. The residue was purified by reverse flash chromatography with the following conditions: column, silica gel; mobile phase, MeCN in water, 5% to 100% gradient in 10 min; detector, UV 254 nm. This resulted 2-(6-(3-methyl-5-(((2-(trifluoromethyl)pyridin-3-yl)oxy)methyl)piperidin-1-yl)pyrazin-2-yl)-1,3,4-thiadiazole (16 mg) as a yellow solid. This product was further purified by prep. HPLC with the following conditions: Column: Xselect CSH F-Phenyl OBD Column 19*150 mm 5 μm, n; Mobile Phase A: Water (0.1% FA), Mobile Phase B: ACN; Flow rate: 25 mL/min; Gradient: 45% B to 57% B in 7 min, 57% B; Wave Length: 254/220 nm; RT1(min): 7.58. This provided 2-(6-((3S,5R)-3-methyl-5-(((2-(trifluoromethyl)pyridin-3-yl)oxy)methyl)piperidin-1-yl)pyrazin-2-yl)-1,3,4-thiadiazole (262a, assumed structure, 7.7 mg, 21.1%) and 2-(6-((3S,5S)-3-methyl-5-(((2-(trifluoromethyl)pyridin-3-yl)oxy)methyl)piperidin-1-yl)pyrazin-2-yl)-1,3,4-thiadiazole (262b, assumed structure, 7.1 mg, 13.8%) as white solids. 262a: ¹H NMR (400 MHZ, CD₃OD) δ 9.43 (s, 1H), 8.53 (s, 1H), 8.27 (s, 1H), 8.12 (d, J=4.6 Hz, 1H), 7.68-7.63 (m, 1H), 7.57-7.51 (m, 1H), 4.14-3.96 (m, 4H), 2.76-2.69 (m, 1H), 2.53-2.46 (m, 1H), 2.13 (d, J=3.9 Hz, 1H), 1.91-1.88 (m, 1H), 1.71 (s, 1H), 1.19 (s, 1H), 0.98-0.94 (m, 3H). MS m/z: 437.0 [M+H]⁺. 262b: ¹H NMR (400 MHZ, CD₃OD) δ 9.39 (s, 1H), 8.44 (s, 1H), 8.21 (s, 1H), 8.04 (d, J=4.6 Hz, 1H), 7.55-7.49 (m, 1H), 7.43 (dd, J=8.6, 4.6 Hz, 1H), 4.11-4.05 (m, 1H), 4.04-3.99 (m, 1H), 3.97-3.90 (m, 2H), 3.67-3.60 (m, 1H), 3.16-3.09 (m, 1H), 2.41-2.33 (m, 1H), 1.98-1.80 (m, 2H), 1.60-1.51 (m, 1H), 0.94 (d, J=6.7 Hz, 3H). MS m/z): 437.0 [M+H]⁺.

### (2,5-dimethyl-6-phenyl-5H-pyrrolo[2,3-b]pyrazin-7-yl)(3-((o-tolyloxy)methyl)piperidin-1-yl)methanone (263)

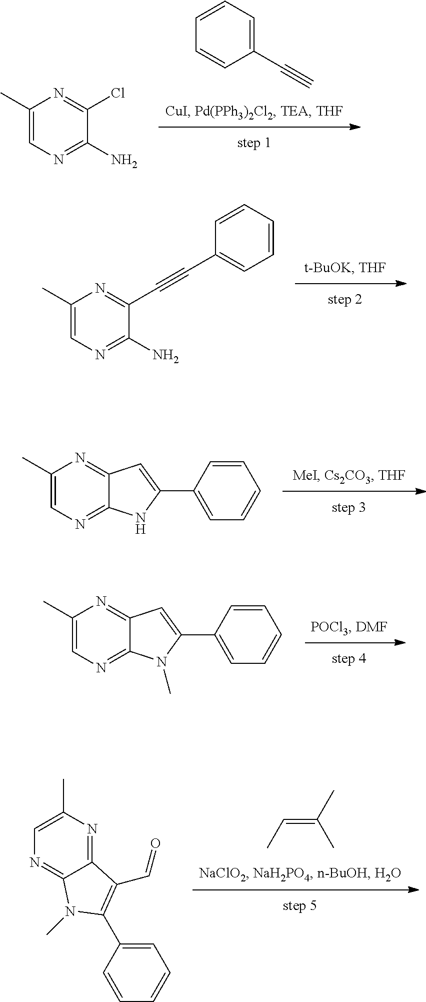

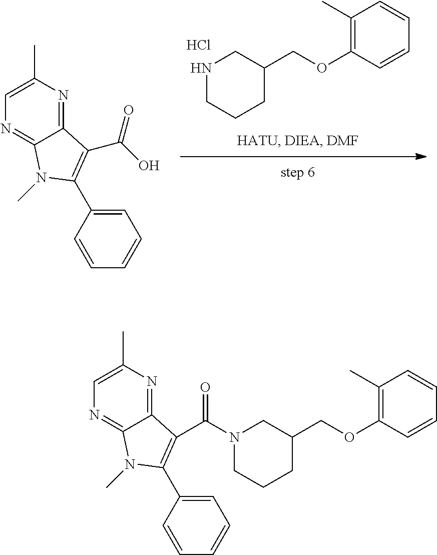 [SMILES: Cc1cnc2c(n1)c(C(=O)O)c(-c1ccccc1)n2C ; Cl ; Cc1ccccc1OCC1CCCNC1 ; Cc1cnc2c(n1)c(C(=O)N1CCCC(COc3ccccc3C)C1)c(-c1ccccc1)n2C]

Step 1: 5-methyl-3-(phenylethynyl)pyrazin-2-amine: A mixture of 3-chloro-5-methylpyrazin-2-amine (1000 mg, 6.96 mmol, 1 equiv) and phenylacetylene (1.42 g, 14.0 mmol, 2 equiv), CuI (132 mg, 0.697 mmol, 0.1 equiv), Pd(PPh₃)₂Cl₂ (489 mg, 0.697 mmol, 0.1 equiv), TEA (212 g, 20.9 mmol, 3 equiv) in THF (8 mL) was stirred for 2 h at 80° C. under inert atmosphere. The mixture was allowed to cool down to room temperature and concentrated. The residue was purified by flash chromatography, eluted with PE/EtOAc (1/1). This provided 5-methyl-3-(2-phenylethynyl)pyrazin-2-amine (1.12 g, 76.8%) as a white solid. MS m/z: 210 [M+H]⁺.

Step 2: 2-methyl-6-phenyl-5H-pyrrolo[2,3-b]pyrazine: A mixture of 5-methyl-3-(2-phenylethynyl)pyrazin-2-amine (1.12 g, 5.35 mmol, 1 equiv) and t-BuOK (0.720 g, 6.42 mmol, 1.2 equiv) in THF (5 mL) was stirred for 2 h at 100° C. under inert atmosphere. The mixture was allowed to cool down to room temperature. The reaction was monitored by LCMS. Desired product could be detected by LCMS. The residue was purified by reverse flash chromatography with the following conditions: column, C18 silica gel, mobile phase, MeCN in Water (0.1% FA), 5% to 100% gradient in 20 min, detector, UV 254 nm. This provided 2-methyl-6-phenyl-5H-pyrrolo[2,3-b]pyrazine (780 mg, 69.6%) as a white solid. MS m/z: 210 [M+H]⁺.

Step 3: 2,5-dimethyl-6-phenyl-5H-pyrrolo[2,3-b]pyrazine: A mixture of 2-methyl-6-phenyl-5H-pyrrolo[2,3-b]pyrazine (780 mg, 3.72 mmol, 1 equiv), MeI (52.9 mg, 0.373 mmol, 0.1 equiv) and Cs₂CO₃ (2.43 g, 7.45 mmol, 2 equiv) in THF (10 mL) was stirred for 16 h at room temperature under air atmosphere. The resulting mixture was concentrated and purified by reverse flash chromatography with the following conditions: column, C18 silica gel, mobile phase, MeCN in Water (0.1% FA), 5% to 100% gradient in 20 min, detector, UV 254 nm. This provided 2,5-dimethyl-6-phenylpyrrolo[2,3-b]pyrazine (470 mg, 56.4%) as a white solid. MS m/z: 224 [M+H]⁺.

Step 4: 2,5-dimethyl-6-phenyl-5H-pyrrolo[2,3-b]pyrazine-7-carbaldehyde: To a stirred solution of POCl₃ (206 mg, 1.34 mmol, 1.5 equiv) in DMF (3 mL) was added 2,5-dimethyl-6-phenylpyrrolo[2,3-b]pyrazine (200 mg, 0.896 mmol, 1 equiv) in portions at 0° C. The resulting mixture was stirred for 2 h at 0° C. under nitrogen atmosphere. The reaction was monitored by LCMS. The mixture was diluted by water (20 mL) neutralized to pH ˜8 with saturated NaHCO₃(aq.). The resulting mixture was extracted with EtOAc (3×10 mL). The combined organic layers were washed with water (2×20 mL) and brine (20 mL), dried over anhydrous Na₂SO₄. After filtration, the filtrate was concentrated under reduced pressure. This provided 2,5-dimethyl-6-phenylpyrrolo[2,3-b]pyrazine-7-carbaldehyde (180 mg, 76.0%) as a yellow solid. MS m/z: 252 [M+H]⁺.

Step 5: 2,5-dimethyl-6-phenyl-5H-pyrrolo[2,3-b]pyrazine-7-carboxylic acid: To a stirred solution of 2,5-dimethyl-6-phenylpyrrolo[2,3-b]pyrazine-7-carbaldehyde (180 mg, 0.717 mmol, 1.00 equiv) in H₂O (1.00 mL) was added NaH₂PO₄ (516 mg, 4.30 mmol, 6.00 equiv) at 0° C. under air atmosphere. After 5 minutes, t-BuOH (5.00 mL), 2,3-dimethylbut-2-ene (150 mg, 1.80 mmol, 2.50 equiv) and NaClO₂ (97.2 mg, 0.867 mmol, 1.50 equiv) were added. After 16 hours, reaction mixture was diluted with ethyl acetate (20 mL), washed with water (2×15 mL) and brine (20 mL), then dried over magnesium sulfate, filtered, and concentrated to give 7-bromo-4-chloropyrazolo[1,5-a]pyridine-3-carboxylic acid (115 mg, 60.0%) as a white solid which was used without further purification. MS m/z: 268 [M+H]⁺.

Step 6: (2,5-dimethyl-6-phenyl-5H-pyrrolo[2,3-b]pyrazin-7-yl)(3-((o-tolyloxy)methyl)piperidin-1-yl)methanone: To a stirred mixture of 3-((o-tolyloxy)methyl)piperidine hydrochloride (92.1 mg, 0.449 mmol, 1.2 equiv) and HATU (170 mg, 0.449 mmol, 1.2 equiv) in DMF (1 mL) were added DIEA (145 mg, 1.12 mmol, 3 equiv) and 2,5-dimethyl-6-phenylpyrrolo[2,3-b]pyrazine-7-carboxylic acid (100 mg, 0.374 mmol, 1.00 equiv) in portions at 0° C. under inert atmosphere. The resulting mixture was stirred for 2 h at room temperature under inert atmosphere. The resulting mixture was purified by reverse flash chromatography with the following conditions: column, C18 silica gel, mobile phase, ACN in water, 5% to 100% gradient in 20 min, detector, UV 254 nm. This resulted (2,5-dimethyl-6-phenyl-5H-pyrrolo[2,3-b]pyrazin-7-yl)(3-((o-tolyloxy)methyl)piperidin-1-yl)methanone (112 mg, 63.7%) as a white solid. ¹H NMR (400 MHz, DMSO-d₆) δ 8.41-8.11 (m, 1H), 7.71-7.36 (m, 5H), 7.18-6.81 (m, 3H), 6.81-6.67 (m, 1H), 4.46-4.27 (m, 1H), 3.94-3.83 (m, 1H), 3.81-3.66 (m, 4H), 3.55-3.45 (m, 1H), 2.97-2.86 (m, 1H), 2.85-2.75 (m, 1H), 2.59 (s, 1H), 2.54 (s, 2H), 2.18 (s, 1H), 1.91-1.54 (m, 3H), 1.51 (s, 2H), 1.41-1.22 (m, 2H). MS m/z: 455.20 [M+H]⁺.

### (2-Methoxy-5-methyl-6-phenyl-5H-pyrrolo[2,3-b]pyrazin-7-yl)(3-((o-tolyloxy)methyl)piperidin-1-yl)methanone (264)

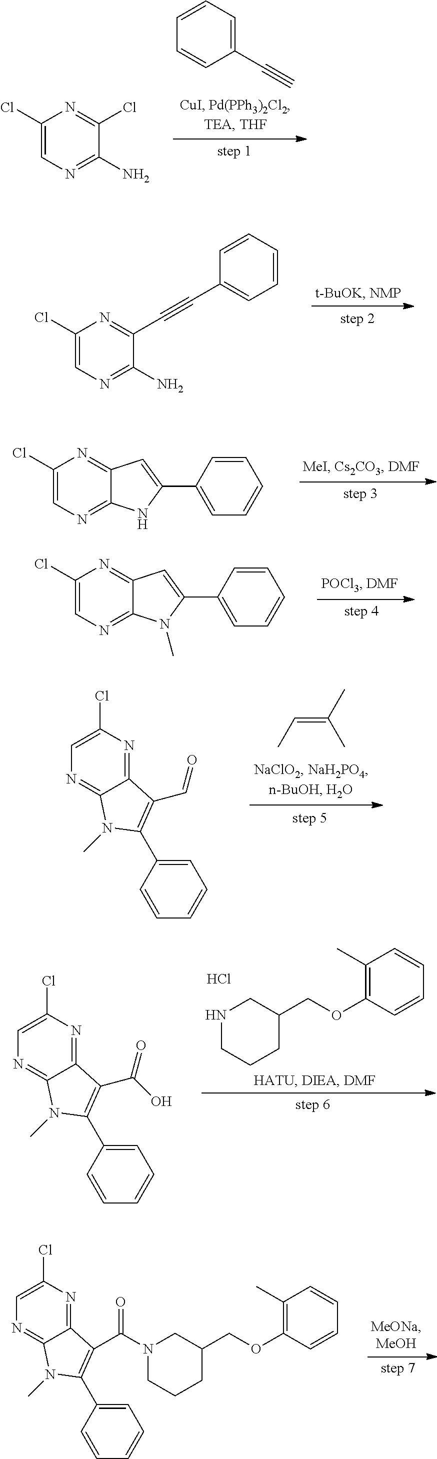

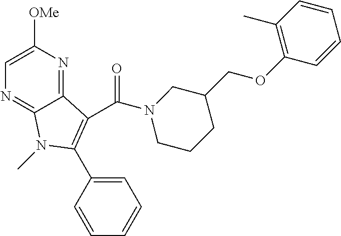 [SMILES: COc1cnc2c(n1)c(C(=O)N1CCCC(COc3ccccc3C)C1)c(-c1ccccc1)n2C]

Step 1: 5-chloro-3-(phenylethynyl)pyrazin-2-amine: A mixture of 3,5-dichloropyrazin-2-amine (1.00 g, 6.10 mmol, 1 equiv) and phenylacetylene (1.25 g, 12.2 mmol, 2 equiv), CuI (117 mg, 0.610 mmol, 0.1 equiv), Pd(PPh₃)₂Cl₂ (428 mg, 0.610 mmol, 0.1 equiv), TEA (1.85 g, 18.3 mmol, 3 equiv) in THF (15 mL) was stirred for 2 h at 80° C. under air atmosphere. The mixture was allowed to cool down to room temperature. The reaction was monitored by LCMS. Desired product could be detected by LCMS. The residue was purified by reverse flash chromatography with the following conditions: column, C18 silica gel, mobile phase, MeCN in Water (0.1% FA), 5% to 100% gradient in 20 min, detector, UV 254 nm. This provided 5-chloro-3-(2-phenylethynyl)pyrazin-2-amine (800 mg, 57.1%) as a white solid. MS m/z: 230 [M-tBu+H]⁺.

Step 2: 2-chloro-6-phenyl-5H-pyrrolo[2,3-b]pyrazine: A mixture of 5-chloro-3-(2-phenylethynyl)pyrazin-2-amine (800 mg, 3.48 mmol, 1 equiv) and t-BuOK (782 mg, 6.97 mmol, 2 equiv) in NMP (5 mL) was stirred for 2 h at 80° C. under air atmosphere. The mixture was allowed to cool down to room temperature. The reaction was monitored by LCMS.

Desired product could be detected by LCMS. The residue was purified by reverse flash chromatography with the following conditions: column, C18 silica gel; mobile phase, MeCN in Water (0.1% FA), 5% to 100% gradient in 20 min, detector, UV 254 nm. This provided 2-chloro-6-phenyl-5H-pyrrolo[2,3-b]pyrazine (560 mg, 70.0%) as a white solid. MS m/z: 230 [M+H]⁺.

Step 3: 2-chloro-5-methyl-6-phenyl-5H-pyrrolo[2,3-b]pyrazine: A mixture of 2-chloro-6-phenyl-5H-pyrrolo[2,3-b]pyrazine (560 mg, 2.44 mmol, 1 equiv) and MeI (34.6 mg, 0.244 mmol, 0.1 equiv), Cs₂CO₃ (1.59 mg, 4.88 mmol, 2 equiv) in DMF (4 mL) was stirred for 16 h at room temperature under air atmosphere. The reaction was monitored by LCMS. Desired product could be detected by LCMS. The residue was purified by reverse flash chromatography with the following conditions: column, C18 silica gel; mobile phase, MeCN in Water (0.1% FA), 5% to 100% gradient in 20 min; detector, UV 254 nm. This provided 2-chloro-5-methyl-6-phenylpyrrolo[2,3-b]pyrazine (450 mg, 75.7%) as a white solid. MS m/z: 244 [M+H]⁺.

Step 4: 2-chloro-5-methyl-6-phenyl-5H-pyrrolo[2,3-b]pyrazine-7-carbaldehyde: To a stirred solution of POCl₃ (206 mg, 1.34 mmol, 1.5 equiv) in DMF (3 mL) was added 2,5-dimethyl-6-phenylpyrrolo[2,3-b]pyrazine (218 mg, 0.896 mmol, 1 equiv) in portions at 0° C. The resulting mixture was stirred for 2 h at 0° C. under nitrogen atmosphere. The reaction was monitored by LCMS. The mixture was diluted by water (20 mL) neutralized to PH ˜8 with saturated NaHCO₃(aq.). The resulting mixture was extracted with EtOAc (3×10 mL). The combined organic layers were washed with water (2×20 mL) and brine (20 mL), dried over anhydrous Na₂SO₄. After filtration, the filtrate was concentrated under reduced pressure. This provided 2,5-dimethyl-6-phenylpyrrolo[2,3-b]pyrazine-7-carbaldehyde (195 mg, 76.0%) as a yellow solid. MS m/z: 272 [M+H]⁺.

Step 5: 2-chloro-5-methyl-6-phenyl-5H-pyrrolo[2,3-b]pyrazine-7-carboxylic acid: To a stirred solution of 2,5-dimethyl-6-phenylpyrrolo[2,3-b]pyrazine-7-carbaldehyde (195 mg, 0.717 mmol, 1.00 equiv) in H₂O (1.00 mL) was added NaH₂PO₄ (516 mg, 4.30 mmol, 6.00 equiv) at 0° C. under air atmosphere. After 5 minutes, t-BuOH (5.00 mL), 2,3-dimethylbut-2-ene (150 mg, 1.80 mmol, 2.50 equiv) and NaClO₂ (97.2 mg, 0.867 mmol, 1.50 equiv) were added. After 16 hours, reaction mixture was diluted with ethyl acetate (20 mL), washed with water (2×15 mL) and brine (20 mL), then dried over magnesium sulfate, filtered, and concentrated to give 7-bromo-4-chloropyrazolo[1,5-a]pyridine-3-carboxylic acid (190 mg, 92.0%) as a white solid which was used without further purification. MS m/z: 288 [M+H]⁺.

Step 6: (2-chloro-5-methyl-6-phenyl-5H-pyrrolo[2,3-b]pyrazin-7-yl)(3-((o-tolyloxy)methyl)piperidin-1-yl)methanone: A mixture of 2-chloro-5-methyl-6-phenylpyrrolo[2,3-b]pyrazine-7-carboxylic acid (190 mg, 0.660 mmol, 1 equiv) and 3-((o-tolyloxy)methyl)piperidine hydrochloride (175 mg, 0.726 mmol, 1.1 equiv), HATU (301 mg, 0.792 mmol, 1.20 equiv), DIEA (256 mg, 1.98 mmol, 3 equiv) in DMF (2 mL) was stirred for 2 h at room temperature under air atmosphere. The reaction was monitored by LCMS. Desired product could be detected by LCMS. The residue was purified by reverse flash chromatography with the following conditions: column, C18 silica gel, mobile phase, MeCN in Water (0.1% FA), 5% to 100% gradient in 20 min; detector, UV 254 nm. This provided 1-{2-chloro-5-methyl-6-phenylpyrrolo[2,3-b]pyrazine-7-carbonyl}-3-(2-methylphenoxymethyl)piperidine (130 mg, 41.4%) as a white solid. MS m/z: 475 [M+H]⁺.

Step 7: (2-methoxy-5-methyl-6-phenyl-5H-pyrrolo[2,3-b]pyrazin-7-yl)(3-((o-tolyloxy)methyl)piperidin-1-yl)methanone: To a stirred mixture of 1-{2-chloro-5-methyl-6-phenylpyrrolo[2,3-b]pyrazine-7-carbonyl}-3-(2-methylphenoxymethyl)piperidine (55.0 mg, 0.116 mmol, 1 equiv) in CH₃OH (2.75 mL, 68.1 mmol, 587 equiv) was added sodium methoxide (1.10 mL, 20.4 mmol, 176 equiv) in portions at 0° C. The resulting mixture was stirred for 3 h at 0° C. under nitrogen atmosphere. The reaction was monitored by LCMS. Desired product could be detected by LCMS. The residue was purified by reverse flash chromatography with the following conditions: column, C18 silica gel, mobile phase, MeCN in Water (0.1% FA), 5% to 100% gradient in 20 min, detector, UV 254 nm. This provided 1-{2-methoxy-5-methyl-6-phenylpyrrolo[2,3-b]pyrazine-7-carbonyl}-3-(2-methylphenoxymethyl)piperidine (43.2 mg, 74.7%) as a white solid. ¹H NMR (400 MHZ, DMSO-d₆) δ 8.07-7.97 (m, 1H), 7.60-7.43 (m, 5H), 7.17-6.71 (m, 4H), 4.38-4.29 (m, 1H), 3.95-3.80 (m, 5H), 3.75-3.69 (m, 3H), 3.59-3.51 (m, 1H), 3.10-2.95 (m, 1H), 2.92-2.75 (m, 1H), 2.21-1.90 (m, 2H), 1.84-1.69 (m, 2H), 1.50 (s, 2H), 1.44-1.20 (m, 2H). MS m/z: 455.20 [M+H]⁺.

### 1-(2-Fluoro-3-methoxypropyl)-6-((3R,5S)-3-methyl-5-(((2-(trifluoromethyl)pyridin-3-yl)oxy)methyl)piperidin-1-yl)-1H-pyrazolo[3,4-b]pyrazine (265)

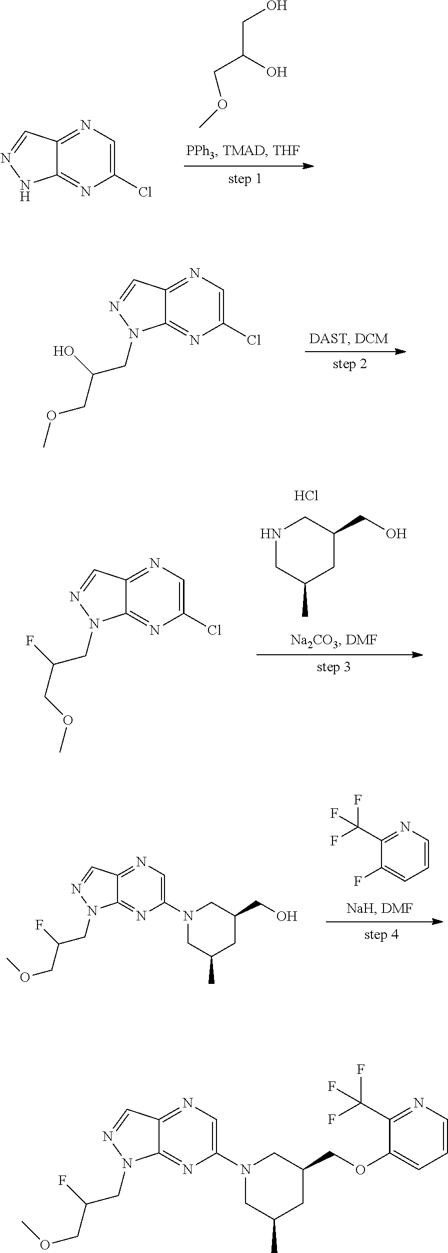

Step 1: 1-(6-chloro-1H-pyrazolo[3,4-b]pyrazin-1-yl)-3-methoxypropan-2-ol: To a stirred mixture of 6-chloro-1H-pyrazolo[3,4-b]pyrazine (200 mg, 1.29 mmol, 1 equiv), 3-methoxypropane-1,2-diol (274 mg, 2.59 mmol, 2 equiv) an PPh₃ (407 mg, 1.55 mmol, 1.2 equiv) in THF (3 mL) was added TMAD (267 mg, 1.55 mmol, 1.2 equiv) in portion at 0° C. The resulting mixture was warmed to 50° C. and stirred for overnight. Desired product could be detected by LCMS. The resulting mixture was concentrated under reduced pressure. The residue was purified by silica gel column chromatography, eluted with EtOAc/PE (1/1) to afford 1-(6-chloro-1H-pyrazolo[3,4-b]pyrazin-1-yl)-3-methoxypropan-2-ol (70 mg, 22.3%) as a yellow solid. MS m/z: 243 [M+H]⁺.

Step 2: 6-chloro-1-(2-fluoro-3-methoxypropyl)-1H-pyrazolo[3,4-b]pyrazine: To a solution of 1-{6-chloropyrazolo[3,4-b]pyrazin-1-yl}-3-methoxypropan-2-ol (50 mg, 0.206 mmol, 1 equiv) in DCM (1 mL) was added DAST (166 mg, 1.03 mmol, 5 equiv) dropwise at 0° C. The mixture was stirred at room temperature for 2 h. The solvent was removed in vacuum. The residue was purified by Prep-TLC (EtOAc/PE=2/3) to afford 6-chloro-1-(2-fluoro-3-methoxypropyl)-1H-pyrazolo[3,4-b]pyrazine (35 mg, 69.4%) as a yellow solid. MS m/z: 245 [M+H]⁺.

Step 3: ((3S,5R)-1-(1-(2-fluoro-3-methoxypropyl)-1H-pyrazolo[3,4-b]pyrazin-6-yl)-5-methylpiperidin-3-yl)methanol: To a stirred solution of 1-(6-chloro-1H-pyrazolo[3,4-b]pyrazin-1-yl)-3-methoxypropan-2-ol (35 mg, 0.143 mmol, 1 equiv) in DMF (1 mL) was added ((3S,5R)-5-methylpiperidin-3-yl)methanol hydrochloride (23.70 mg, 0.143 mmol, 1 equiv) and Na₂CO₃ (45.5 mg, 0.429 mmol, 3 equiv) sequentially at room temperature. The mixture was stirred for 2 h at 50° C. The resulting mixture was diluted with EA (20 mL), washed with water (1×20 mL) and brine (1×20 mL), and was dried over by anhydrous sodium sulfate. After filtration, the filtrate was concentrated by reduced pressure. The residue was purified by silica gel column chromatography, eluted with EA/PE (1/1) to afford ((3S,5R)-1-(1-(2-fluoro-3-methoxypropyl)-1H-pyrazolo[3,4-b]pyrazin-6-yl)-5-methylpiperidin-3-yl)methanol (20 mg, 41.43%) as a white solid. MS m/z: 338 [M+H]⁺.

Step 4: 1-(2-fluoro-3-methoxypropyl)-6-((3R,5S)-3-methyl-5-(((2-(trifluoromethyl)pyridin-3-yl)oxy)methyl)piperidin-1-yl)-1H-pyrazolo[3,4-b]pyrazine: To a solution of ((3S,5R)-1-(1-(2-fluoro-3-methoxypropyl)-1H-pyrazolo[3,4-b]pyrazin-6-yl)-5-methylpiperidin-3-yl)methanol (20 mg, 0.059 mmol, 1.00 equiv) in DMF (1 mL), NaH (2.85 mg, 0.118 mmol, 2 equiv) was added in portions at 0° C. under inert atmosphere. The mixture was reacted at this temperature for 30 min. Then to the mixture, 3-fluoro-2-(trifluoromethyl)pyridine (19.5 mg, 0.118 mmol, 2 equiv) in DMF (0.5 mL) was added in one pot. The reaction was allowed to warm to the room temperature and reacted for 16 h. The reaction was quenched by 10 mL water at 0° C. The aqueous layer was washed by EtOAc (10 mL×3). The combined organic layer was dried over sodium sulfate. After removing the organic solvent, the residue was purified by reverse flash chromatography with the following conditions: column, silica gel; mobile phase, MeCN in water, 35% to 80% gradient in 20 min; detector, UV 254 nm. This provided 1-(2-fluoro-3-methoxypropyl)-6-((3R,5S)-3-methyl-5-(((2-(trifluoromethyl)pyridin-3-yl)oxy)methyl)piperidin-1-yl)-1H-pyrazolo[3,4-b]pyrazine (15 mg, 52.45%) as a white solid. ¹H NMR (400 MHZ, DMSO-d₆) δ 8.43 (d, J=1.0 Hz, 1H), 8.27 (d, J=4.5 Hz, 1H), 8.09 (s, 1H), 7.83 (d, J=8.6 Hz, 1H), 7.71 (dd, J=8.6, 4.5 Hz, 1H), 5.10 (m, 5.22-4.96, 1H), 4.86 (4.93-4.79, 1H), 4.67-4.32 (m, 3H), 4.27-4.16 (m, 1H), 4.05 (t, J=8.7 Hz, 1H), 3.74-3.47 (m, 2H), 3.29 (s, 3H), 2.80-2.64 (m, 1H), 2.61-2.54 (m, 1H), 2.20-2.01 (m, 1H), 1.96-1.83 (m, 1H), 1.79-1.64 (m, 1H), 1.08 (q, J=12.2 Hz, 1H), 0.98 (d, J=6.5 Hz, 3H). MS m/z: 483.2 [M+H]⁺.

### 1-(2,2-Difluoroethyl)-6-((S)-3-((R)-1-(2-(trifluoromethyl)phenoxy)ethyl)piperidin-1-yl)-1H-pyrazolo[3,4-b]pyrazine (266a) and 1-(2,2-difluoroethyl)-6-((R)-3-((S)-1-(2-(trifluoromethyl)phenoxy)ethyl)piperidin-1-yl)-1H-pyrazolo[3,4-b]pyrazine (266b)

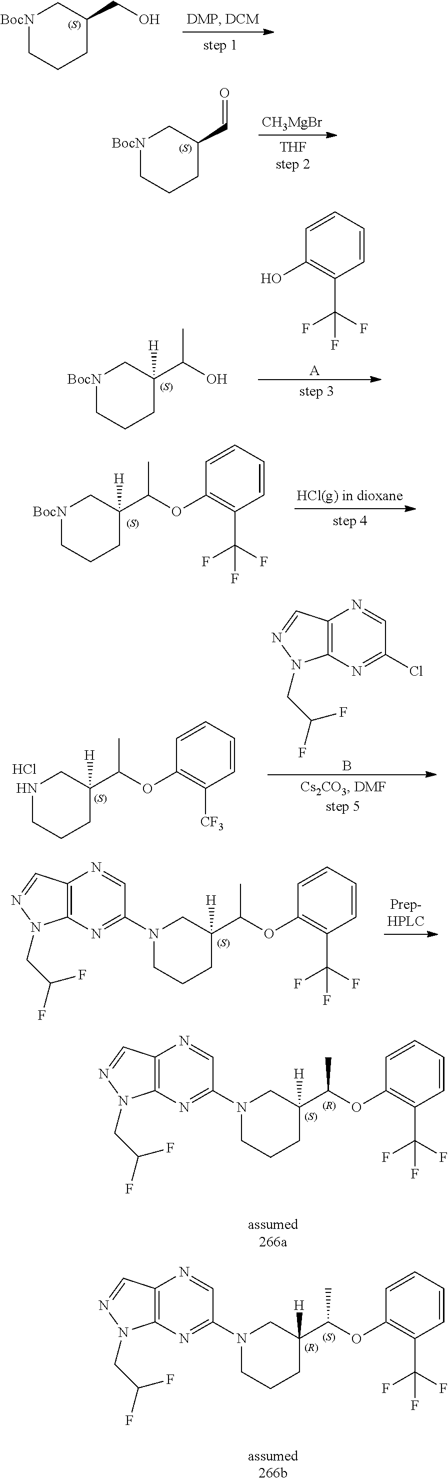

Step 1: tert-butyl (S)-3-formylpiperidine-1-carboxylate: To a stirred solution of tert-butyl tert-butyl (S)-3-(hydroxymethyl)piperidine-1-carboxylate (2 g, 9.29 mmol, 1 equiv) in DCM (20 mL) was added DMP (4.73 g, 11.1 mmol, 1.2 equiv) at 0° C. The resulting mixture was stirred for 2 h at room temperature. Desired product could be detected by LCMS. The resulting mixture was concentrated under reduced pressure. The residue was purified by silica gel column chromatography, eluted with PE/EA (1:1) to afford tert-butyl (3S)-3-formylpiperidine-1-carboxylate (1.3 g, 65.6%) as a colorless oil. MS m/z: 214 [M+H]⁺.

Step 2: tert-butyl (3S)-3-(1-hydroxyethyl)piperidine-1-carboxylate: To a stirred solution of tert-butyl (R)-3-formylpiperidine-1-carboxylate (1.3 g, 6.09 mmol, 1 equiv) in THF (10 mL) was added CH₃MgBr (2.44 mL, 7.31 mmol, 1.2 equiv) dropwise at 0° C. The resulting mixture was stirred for 2 h at room temperature. Desired product could be detected by LCMS. The reaction was quenched by the addition of sat. NH₄Cl (aq.) (10 mL) at 0° C. The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (1:1) to afford tert-butyl (3R)-3-(1-hydroxyethyl)piperidine-1-carboxylate (700 mg, 50.0%) as a colorless oil. MS m/z: 230 [M+H]⁺.

Step 3: tert-butyl (3S)-3-(1-(2-(trifluoromethyl)phenoxy)ethyl)piperidine-1-carboxylate: To a stirred mixture of tert-butyl tert-butyl (3S)-3-(1-hydroxyethyl)piperidine-1-carboxylate (700 mg, 3.05 mmol, 1 equiv), 2-(trifluoromethyl)phenol (544 mg, 3.35 mmol, 1.1 equiv) and PPh₃ (1200 mg, 4.57 mmol, 1.5 equiv) in THF (6 mL) was added TMAD (788 mg, 4.57 mmol, 1.5 equiv) in portion at 0° C. The resulting mixture was warmed to room temperature and stirred for overnight at room temperature. Desired product could be detected by LCMS. The resulting mixture was concentrated under reduced pressure. The residue was purified by silica gel column chromatography, eluted with EtOAc/PE (1/2) to afford tert-butyl (3S)-3-(1-(2-(trifluoromethyl)phenoxy)ethyl)piperidine-1-carboxylate (330 mg, 28.9%) as a colorless oil. MS m/z: 374 [M+H]⁺.

Step 4: (3S)-3-(1-(2-((difluoro-13-methyl)-12-fluoraneyl)phenoxy)ethyl)piperidine hydrochloride: Tert-butyl (3S)-3-(1-(2-(trifluoromethyl)phenoxy)ethyl)piperidine-1-carboxylate (330 mg, 0.884 mmol, 1 equiv) was dissolved in DCM (3 mL)/HCl(gas) in dioxane (3 mL). The mixture was stirred at room temperature for 2 h. After removing the solvent, the crude product (3S)-3-(1-(2-((difluoro-13-methyl)-12-fluoraneyl)phenoxy)ethyl)piperidine hydrochloride (200 mg) was used for next step without further purification. MS m/z: 274 [M+H]⁺.

Step 5: 1-(2,2-difluoroethyl)-6-((3S)-3-(1-(2-(trifluoromethyl)phenoxy)ethyl)piperidin-1-yl)-1H-pyrazolo[3,4-b]pyrazine: A solution of (3S)-3-(1-(2-((difluoro-13-methyl)-12-fluoraneyl)phenoxy)ethyl)piperidine hydrochloride (200 mg, 0.644 mmol, 1 equiv) and 6-chloro-1-(2,2-difluoroethyl)-1H-pyrazolo[3,4-b]pyrazine (154 mg, 0.708 mmol, 1.1 equiv) and Cs₂CO₃ (629 mg, 1.93 mmol, 3 equiv) in DMF (3 mL) was stirred for 2 h at 100° C. The residue was purified by reverse flash chromatography with the following conditions: column, silica gel; mobile phase, MeCN in water, 10% to 50% gradient in 10 min; detector, UV 254 nm. This provided 1-(2,2-difluoroethyl)-6-((3S)-3-(1-(2-(trifluoromethyl)phenoxy)ethyl)piperidin-1-yl)-1H-pyrazolo[3,4-b]pyrazine (70 mg) as a white solid. This product was further purified by prep. Chiral HPLC with the following conditions: column: Column: Lux 5 um Cellulose-4, 2.12*25 cm, 5 um; Mobile Phase A: Water (0.05% DEA), Mobile Phase B: ACN; Flow rate: 20 mL/min; Gradient: 60% B to 60% B in 20 min; Wave Length: 198/252 nm; RT1(min): 18; RT2(min): 19; Sample Solvent: MeOH-HPLC; Injection Volume: 0.2 mL. This provided 1-(2,2-difluoroethyl)-6-((S)-3-((R)-1-(2-(trifluoromethyl)phenoxy)ethyl)piperidin-1-yl)-1H-pyrazolo[3,4-b]pyrazine (266a, 15 mg, 21.4%) as a colorless oil and 1-(2,2-difluoroethyl)-6-((R)-3-((S)-1-(2-(trifluoromethyl)phenoxy)ethyl)piperidin-1-yl)-1H-pyrazolo[3,4-b]pyrazine (266b, 10 mg, 14.2%) as a light yellow solid.

266a: ¹H NMR (400 MHZ, DMSO-d₆) δ 8.39 (s, 1H), 8.11 (s, 1H), 7.66-7.54 (m, 2H), 7.27 (d, J=8.5 Hz, 1H), 7.06 (t, J=7.6 Hz, 1H), 6.54-6.22 (m, 1H), 4.76-4.54 (m, 4H), 4.41 (d, J=13.5 Hz, 1H), 3.08 (t, J=11.4 Hz, 1H), 3.01-2.92 (m, 1H), 1.93-1.77 (m, 3H), 1.59-1.40 (m, 2H), 1.30 (d, J=6.0 Hz, 3H). MS m/z: 456.2 [M+H]⁺.

266b: ¹H NMR (400 MHZ, DMSO-d₆) δ 8.38 (s, 1H), 8.11 (s, 1H), 7.66-7.54 (m, 2H), 7.27 (d, J=8.5 Hz, 1H), 7.06 (t, J=7.6 Hz, 1H), 6.55-6.22 (m, 1H), 4.76-4.54 (m, 4H), 4.41 (d, J=13.4 Hz, 1H), 3.13-2.92 (m, 2H), 1.93-1.77 (m, 3H), 1.60-1.42 (m, 2H), 1.29 (d, J=6.1 Hz, 3H). MS m/z: 456.1[M+H]⁺.

### (2-(5-Fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (267)

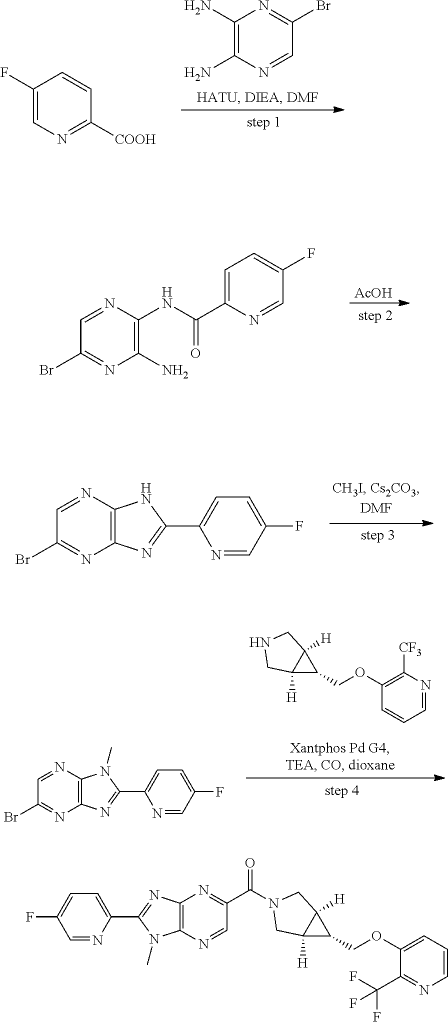

Step 1: N-(3-amino-5-bromopyrazin-2-yl)-5-fluoropicolinamide: A solution of 5-fluoropyridine-2-carboxylic acid (1.5 g, 10.6 mmol, 1 equiv), 5-bromopyrazine-2,3-diamine (3.21 g, 17.0 mmol, 1.6 equiv), HATU (6.06 g, 15.9 mmol, 1.5 equiv) and DIPEA (2.75 g, 21.3 mmol, 2 equiv) in DMF (10 mL) was stirred for 2 days at 70° C. The residue was purified by reverse flash chromatography with the following conditions: column, C18 silica gel; mobile phase, MeCN in water, 10% to 100% gradient in 30 min; detector, UV 254 nm. This provided N-(3-amino-5-bromopyrazin-2-yl)-5-fluoropyridine-2-carboxamide (1.2 g, 36.2%) as a white solid. MS m/z: 312 [M+H]⁺.

Step 2: 5-bromo-2-(5-fluoropyridin-2-yl)-1H-imidazo[4,5-b]pyrazine: A solution of N-(3-amino-5-bromopyrazin-2-yl)-5-fluoropyridine-2-carboxamide (1.2 g, 3.84 mmol, 1 eqiv) in AcOH (10 mL) was stirred for 3 days at 100° C. The reaction was quenched with Na₂CO₃(aq.) at 0° C. The resulting mixture was concentrated under vacuum. The residue was purified by reverse flash chromatography with the following conditions: column, C18 silica gel; mobile phase, MeCN in water, 10% to 100% gradient in 30 min; detector, UV 254 nm. This provided 5-bromo-2-(5-fluoropyridin-2-yl)-1H-imidazo[4,5-b]pyrazine (300 mg, 26.5%) as a white solid. MS m/z: 294 [M+H]⁺.

Step 3. 5-bromo-2-(5-fluoropyridin-2-yl)-1-methyl-1H-imidazo[4,5-b]pyrazine: A solution of 5-bromo-2-(5-fluoropyridin-2-yl)-1H-imidazo[4,5-b]pyrazine (300 mg, 1.02 mmol, 1 equiv), CH₃I (159 mg, 1.12 mmol, 1.1 equiv) and Cs₂CO₃ (665 mg, 2.04 mmol, 2 equiv) in DMF (5 mL) was stirred for overnight at room temperature. The residue was purified by reverse flash chromatography with the following conditions: column, C18 silica gel; mobile phase, MeCN in water, 10% to 100% gradient in 30 min; detector, UV 254 nm. MS m/z: 308 [M+H]⁺.

Step 4. (2-(5-fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone: A solution of 5-bromo-2-(5-fluoropyridin-2-yl)-1-methyl-1H-imidazo[4,5-b]pyrazine (40 mg, 0.130 mmol, 1 equiv), (1R,5S,6S)-6-({[2-(trifluoromethyl)pyridin-3-yl]oxy}methyl)-3-azabicyclo[3.1.0]hexane (33.5 mg, 0.130 mmol, 1 equiv), Xantphos Pd G4 (12.5 mg, 0.013 mmol, 0.1 equiv) and TEA (26.3 mg, 0.260 mmol, 2 equiv) in dioxane (2 mL) was stirred for overnight at 50° C. under carbon monoxide atmosphere. The resulting mixture was concentrated under reduced pressure. The residue was purified by silica gel column chromatography, eluted with PE/EA (1:1) to afford impure product. This was further purified by reverse flash chromatography with the following conditions: column, C18 silica gel; mobile phase, MeCN in water, 10% to 100% gradient in 30 min; detector, UV 254 nm. This provided (2-(5-fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (8 mg 12.0%) as a white solid. ¹H NMR (400 MHZ, DMSO-d₆) δ 8.87 (d, J=2.9 Hz, 1H), 8.82 (s, 1H), 8.56 (dd, J=8.9, 4.6 Hz, 1H), 8.25 (d, J=4.4 Hz, 1H), 8.06 (td, J=8.7. 2.9 Hz, 1H), 7.80 (d, J=8.6 Hz, 1H), 7.68 (dd, J=8.6, 4.5 Hz, 1H), 4.26 (s, 3H), 4.23-4.11 (m, 2H), 4.02-3.91 (m, 3H), 3.61 (dd, J=12.3, 4.0 Hz, 1H), 1.87-1.75 (m, 2H), 1.18-1.08 (m, 1H). MS m/z: 513.9 [M+H]⁺.

### (2-(5-Fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (268)

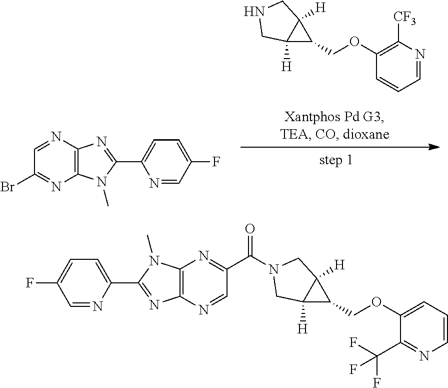 [SMILES: FC(F)(F)c1ncccc1OC[C@H]1[C@@H]2CNC[C@@H]21 ; Cn1c(-c2ccc(F)cn2)nc2ncc(Br)nc21 ; Cn1c(-c2ccc(F)cn2)nc2ncc(C(=O)N3C[C@@H]4[C@@H](COc5cccnc5C(F)(F)F)[C@@H]4C3)nc21]

A solution of 2-{6-bromo-1-methylimidazo[4,5-b]pyridin-2-yl}-5-fluoropyridine (40 mg, 0.13 mmol, 1 equiv), TEA (26.4 mg, 0.26 mmol, 2 equiv), Xantphos Pd G3 (12.5 mg, 0.01 mmol, 0.1 equiv) and (1R,5S,6S)-6-({[2-(trifluoromethyl)pyridin-3-yl]oxy}methyl)-3-azabicyclo[3.1.0]hexane (33.6 mg, 0.13 mmol, 1 equiv) in dioxane (3 mL) was stirred for overnight at 50° C. under carbon monoxide atmosphere. The resulting mixture was concentrated under vacuum. The residue was purified by silica gel column chromatography, eluted with PE/EA (1:1) to afford impure product. This was purified by reverse flash chromatography with the following conditions: column, C18 silica gel; mobile phase, MeCN in water, 10% to 100% gradient in 30 min; detector, UV 254 nm. This provided (2-(5-fluoropyridin-2-yl)-1-methyl-1H-imidazo[4,5-b]pyrazin-5-yl)((1R,5S,6r)-6-(((2-(trifluoromethyl)pyridin-3-yl)oxy)methyl)-3-azabicyclo[3.1.0]hexan-3-yl)methanone (8 mg, 11.60%) as a white solid. ¹H NMR (400 MHZ, DMSO-d₆) δ 8.95-8.84 (m, 2H), 8.55 (dd, J=8.9, 4.6 Hz, 1H), 8.25 (d, J=4.4 Hz, 1H), 8.11-8.01 (m, 1H), 7.80 (d, J=8.6 Hz, 1H), 7.67 (dd, J=8.5, 4.5 Hz, 1H), 4.25 (s, 3H), 4.22-4.12 (m, 2H), 4.06-3.99 (m, 2H), 3.95 (d, J=11.6 Hz, 1H), 3.66-3.57 (m, 1H), 1.85-1.76 (m, 2H), 1.17-1.14 (m, 1H). MS m/z: 514.2 [M+H]⁺.

### 1-(2,2-Difluoroethyl)-6-((2R,5S)-2-methyl-5-(((6-(trifluoromethyl)pyridin-2-yl)oxy)methyl)piperidin-1-yl)-1H-pyrazolo[3,4-b]pyrazine (269)

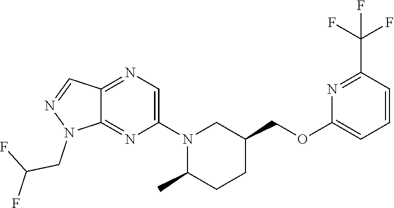 [SMILES: C[C@@H]1CC[C@H](COc2cccc(C(F)(F)F)n2)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30.0 mg, 0.096 mmol, 1.00 equiv) and 2-fluoro-6-(trifluoromethyl)pyridine (23.9 mg, 0.144 mmol, 1.50 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided 1-(2,2-difluoroethyl)-6-((2R,5S)-2-methyl-5-(((6-(trifluoromethyl)pyridin-2-yl)oxy)methyl)piperidin-1-yl)-1H-pyrazolo[3,4-b]pyrazine (24.0 mg, 54.6%) as a colorless oil. ¹H NMR (400 MHZ, DMSO-d₆) δ 8.45 (s, 1H), 8.12 (s, 1H), 8.02-7.97 (m, 1H), 7.51-7.49 (m, 1H), 7.23-7.21 (m, 1H), 6.57-6.28 (m, 1H), 4.90 (s, 1H), 4.83-4.50 (m, 3H), 4.40-4.22 (m, 2H), 2.95-2.89 (m, 1H), 2.07-2.04 (m, 1H), 1.86-1.52 (m, 4H), 1.22-1.20 (d, J=6.8 Hz, 3H). MS m/z: 457.1 [M+H]⁺.

### 3-{5-Methyl-6-phenyl-5H-pyrrolo[2,3-b]pyrazine-7-carbonyl}-1-({[2-(trifluoromethyl)pyridin-3-yl]oxy}methyl)-3-azabicyclo[3.1.0]hexane (270)

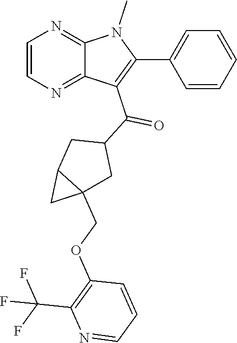 [SMILES: Cn1c(-c2ccccc2)c(C(=O)C2CC3CC3(COc3cccnc3C(F)(F)F)C2)c2nccnc21]

Followed General Procedure E using 5-methyl-6-phenyl-5H-pyrrolo[2,3-b]pyrazine-7-carboxylic acid (20 mg, 0.08 mmol) and 1-({[2-(trifluoromethyl)pyridin-3-yl]oxy}methyl)-3-azabicyclo[3.1.0]hexane (20.4 mg, 0.08 mmol) to afford 3-{5-methyl-6-phenyl-5H-pyrrolo[2,3-b]pyrazine-7-carbonyl}-1-({[2-(trifluoromethyl)pyridin-3-yl]oxy}methyl)-3-azabicyclo[3.1.0]hexane (22 mg, 56%) as a colorless oil. ¹H NMR (500 MHz, CDCl₃) δ 8.51 (d, J=2.6 Hz, 1H), 8.30 (t, J=2.5 Hz, 1H), 8.28-8.25 (m, 1H), 7.60-7.49 (m, 4H), 7.47-7.42 (m, 1H), 7.40 (dd, J=8.5, 4.6 Hz, 1H), 7.30 (d, J=8.5 Hz, 1H), 4.30-4.24 (m, 1H), 4.10 (s, 1H), 4.04 (t, J=9.5 Hz, 1H), 3.89 (d, J=9.7 Hz, 1H), 3.83 (s, 3H), 3.57-3.53 (m, 1H), 3.39-3.34 (m, 2H), 3.29 (dd, J=10.7, 4.0 Hz, 1H), 1.54 (dd, J=8.3, 4.2 Hz, 1H), 0.84 (t, J=6.8 Hz, 1H), 0.42 (t, J=4.9 Hz, 1H). MS m/z: 494.6 [M+H]⁺.

### (1R,5S,6R)-3-{5-Methyl-6-phenyl-5H-pyrrolo[2,3-b]pyrazine-7-carbonyl}-6-({[2-(trifluoromethyl)pyridin-3-yl]oxy}methyl)-3-azabicyclo[3.1.0]hexane (271)

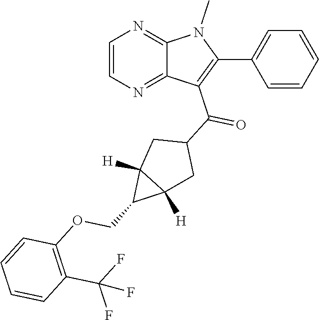 [SMILES: Cn1c(-c2ccccc2)c(C(=O)C2C[C@@H]3[C@H](COc4ccccc4C(F)(F)F)[C@@H]3C2)c2nccnc21]

Followed General Procedure E using 5-methyl-6-phenyl-5H-pyrrolo[2,3-b]pyrazine-7-carboxylic acid (20 mg, 0.08 mmol) and (1R,5S,6R)-6-({[2-(trifluoromethyl)pyridin-3-yl]oxy}methyl)-3-azabicyclo[3.1.0]hexane hydrochloride (30 mg, 0.1 mmol) to afford (1R,5S,6R)-3-{5-methyl-6-phenyl-5H-pyrrolo[2,3-b]pyrazine-7-carbonyl}-6-({[2-(trifluoromethyl)pyridin-3-yl]oxy}methyl)-3-azabicyclo[3.1.0]hexane (10 mg, 20%) as a white solid. ¹H NMR (500 MHZ, DMSO-d₆) δ 8.33-8.27 (m, 2H), 7.95 (d, J=2.6 Hz, 1H), 7.84 (dd, J=8.7, 1.2 Hz, 1H), 7.77 (dd, J=8.5, 4.5 Hz, 1H), 7.58-7.52 (m, 2H), 7.48 (dd, J=5.1, 1.8 Hz, 3H), 4.35 (dd, J=10.7, 7.4 Hz, 1H), 4.19 (dd, J=10.6, 8.3 Hz, 1H), 3.71 (s, 3H), 3.67 (d, J=12.8 Hz, 1H), 3.64-3.55 (m, 2H), 3.47 (d, J=11.1 Hz, 1H), 1.90 (dd, J=7.8, 5.0 Hz, 1H), 1.83 (td, J=7.7, 5.1 Hz, 1H), 1.46 (p, J=8.0 Hz, 1H). MS m/z: 494.6 [M+H]⁺.

### 1-(2,2-Difluoroethyl)-6-((2R,5S)-2-methyl-5-(((6-(trifluoromethyl)pyridin-3-yl)oxy)methyl)piperidin-1-yl)-1H-pyrazolo[3,4-b]pyrazine (272)

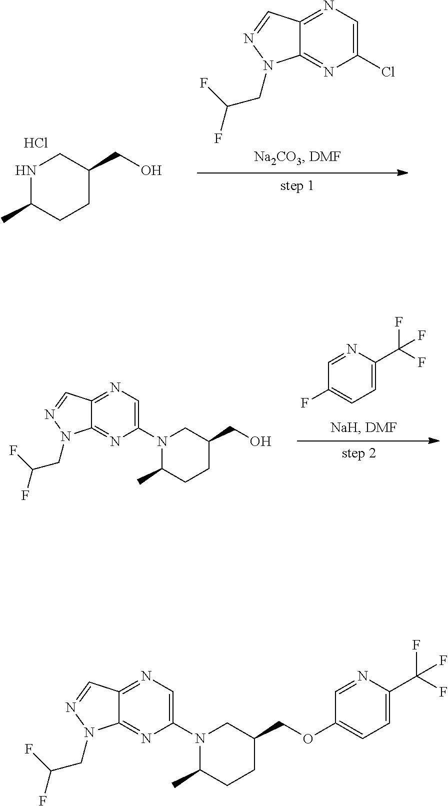

Step 1: ((3S,6R)-1-(1-(2,2-difluoroethyl)-1H-pyrazolo[3,4-b]pyrazin-6-yl)-6-methylpiperidin-3-yl)methanol: Followed General Procedure C using ((3S,6R)-6-methylpiperidin-3-yl)methanol hydrochloride (250 mg, 1.51 mmol, 1.00 equiv) and 6-chloro-1-(2,2-difluoroethyl)-1H-pyrazolo[3,4-b]pyrazine (330 mg, 1.51 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided ((3S,6R)-1-(1-(2,2-difluoroethyl)-1H-pyrazolo[3,4-b]pyrazin-6-yl)-6-methylpiperidin-3-yl)methanol (400 mg, 85.1%) as a yellow solid. MS m/z: 312 [M+H]⁺.

Step 2: 1-(2,2-difluoroethyl)-6-((2R,5S)-2-methyl-5-(((6-(trifluoromethyl)pyridin-3-yl)oxy)methyl)piperidin-1-yl)-1H-pyrazolo[3,4-b]pyrazine: Followed General Procedure D using ((3S,6R)-1-(1-(2,2-difluoroethyl)-1H-pyrazolo[3,4-b]pyrazin-6-yl)-6-methylpiperidin-3-yl)methanol (30.0 mg, 0.096 mmol, 1.00 equiv) and 5-fluoro-2-(trifluoromethyl)pyridine (23.9 mg, 0.144 mmol, 1.50 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided 1-(2,2-difluoroethyl)-6-((2R,5S)-2-methyl-5-(((6-(trifluoromethyl)pyridin-3-yl)oxy)methyl)piperidin-1-yl)-1H-pyrazolo[3,4-b]pyrazine (20.0 mg, 45.5%) as a colorless oil. ¹H NMR (300 MHZ, DMSO-d₆) δ 8.53-8.52 (m, 1H), 8.47 (s, 1H), 8.13 (s, 1H), 7.89-7.86 (m, 1H), 7.70-7.66 (m, 1H), 6.65-6.23 (m, 1H), 4.91 (s, 1H), 4.74-4.58 (m, 3H), 4.20-4.10 (m, 2H), 2.97-2.89 (m, 1H), 2.07 (s, 1H), 1.82-1.75 (m, 3H), 1.67-1.60 (m, 1H), 1.24-1.22 (m, 3H). MS m/z: 457.1 [M+H]⁺.

### 1-(2,2-Difluoroethyl)-6-((2R,5S)-2-methyl-5-(((5-(trifluoromethyl)pyridin-3-yl)oxy)methyl)piperidin-1-yl)-1H-pyrazolo[3,4-b]pyrazine (275)

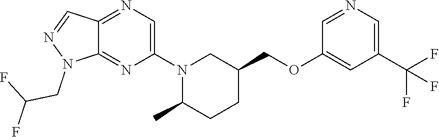 [SMILES: C[C@@H]1CC[C@H](COc2cncc(C(F)(F)F)c2)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30.0 mg, 0.096 mmol, 1.00 equiv) and 3-fluoro-5-(trifluoromethyl)pyridine (23.9 mg, 0.144 mmol, 1.50 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided 1-(2,2-difluoroethyl)-6-((2R,5S)-2-methyl-5-(((5-(trifluoromethyl)pyridin-3-yl)oxy)methyl)piperidin-1-yl)-1H-pyrazolo[3,4-b]pyrazine (20.0 mg, 45.5%) as a colorless oil. ¹H NMR (300 MHz, DMSO-d₆) δ 8.68-8.67 (m, 1H), 8.58 (s, 1H), 8.47 (s, 1H), 8.14 (s, 1H), 7.84 (s, 1H), 6.64-6.25 (m, 1H), 4.91 (s, 1H), 4.75-4.52 (m, 3H), 4.27-4.10 (m, 2H), 2.97-2.88 (m, 1H), 2.06 (s, 1H), 1.89-1.55 (m, 4H), 1.24-1.21 (m, 3H). MS m/z: 457.1 [M+H]⁺.

### 1-(2,2-Difluoroethyl)-6-((2R,5S)-2-methyl-5-(((4-(trifluoromethyl)pyridin-3-yl)oxy)methyl)piperidin-1-yl)-1H-pyrazolo[3,4-b]pyrazine (274)

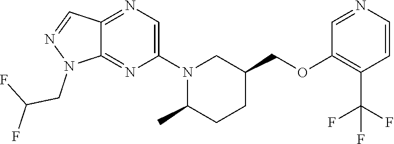 [SMILES: C[C@@H]1CC[C@H](COc2cnccc2C(F)(F)F)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30 mg, 0.096 mmol, 1 equiv) and 3-fluoro-4-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((4-(trifluoromethyl)pyridin-3-yl)oxy)methyl)piperidin-1-yl)-1H-pyrazolo[3,4-b]pyrazine (30 mg, 68.21%) as a white solid. ¹H NMR (300 MHz, DMSO-d₆) δ 8.74 (s, 1H), 8.51-8.37 (m, 2H), 8.13 (s, 1H), 7.69-7.67 (m, 1H), 6.61-6.22 (m, 1H), 4.88-4.86 (m, 1H), 4.81-4.57 (m, 3H), 4.43-4.39 (m, 1H), 4.26-4.20 (m, 1H), 2.98-2.82 (m, 1H), 2.08 (s, 2H), 1.91-1.57 (m, 4H), 1.24-1.22 (m, 3H). MS m/z: 457.1 [M+H]⁺.

### 1-(2,2-Difluoroethyl)-6-((2R,5S)-2-methyl-5-(((5-(trifluoromethyl)pyridin-2-yl)oxy)methyl)piperidin-1-yl)-1H-pyrazolo[3,4-b]pyrazine (275)

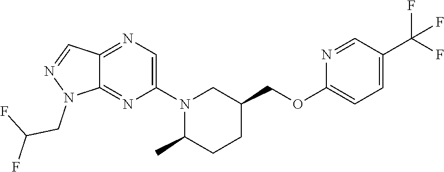 [SMILES: C[C@@H]1CC[C@H](COc2ccc(C(F)(F)F)cn2)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30 mg, 0.096 mmol, 1 equiv) and 2-fluoro-5-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((5-(trifluoromethyl)pyridin-2-yl)oxy)methyl)piperidin-1-yl)-1H-pyrazolo[3,4-b]pyrazine (20 mg, 45.48%) as a white solid. ¹H NMR (300 MHz, DMSO-d₆) δ 8.61 (s, 1H), 8.45 (s, 1H), 8.13 (s, 1H), 8.12-8.08 (m, 1H), 7.12-7.09 (m, 1H), 6.63-6.23 (m, 1H), 4.90 (s, 1H), 4.79-4.47 (m, 3H), 4.38-4.36 (m, 2H), 2.95-2.87 (m, 1H), 2.06 (s, 1H), 1.92-1.51 (m, 4H), 1.23-1.15 (m, 3H). MS m/z: 457.1 [M+H]⁺.

### 1-(2,2-Difluoroethyl)-6-((2R,5S)-2-methyl-5-(((4-(trifluoromethyl)pyridin-2-yl)oxy)methyl)piperidin-1-yl)-1H-pyrazolo[3,4-b]pyrazine (276)

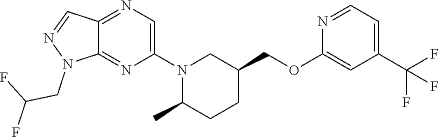 [SMILES: C[C@@H]1CC[C@H](COc2cc(C(F)(F)F)ccn2)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol and 2-fluoro-4-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((4-(trifluoromethyl)pyridin-2-yl)oxy)methyl)piperidin-1-yl)-1H-pyrazolo[3,4-b]pyrazine (20 mg, 45.5%) as a colorless oil. ¹H NMR (400 MHZ, DMSO-d₆) δ 8.46-8.44 (m, 2H), 8.12 (s, 1H), 7.35-7.33 (m, 1H), 7.28-7.28 (m, 1H), 6.57-6.28 (m, 1H), 4.92-4.90 (m, 1H), 4.71-4.62 (m, 2H), 4.61-4.54 (m, 1H), 4.42-4.29 (m, 2H), 2.95-2.89 (m, 1H), 2.08-2.05 (m, 1H), 1.86-1.68 (m, 3H), 1.68-1.54 (m, 1H), 1.23-1.15 (m, 3H). MS m/z: 457.1 [M+H]⁺.

### 1-(2,2-Difluoroethyl)-6-((2R,5S)-2-methyl-5-(((3-(trifluoromethyl)pyridin-2-yl)oxy)methyl)piperidin-1-yl)-1H-pyrazolo[3,4-b]pyrazine (277)

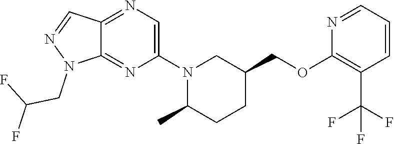 [SMILES: C[C@@H]1CC[C@H](COc2ncccc2C(F)(F)F)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30 mg, 0.096 mmol, 1 equiv) and 2-fluoro-3-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((3-(trifluoromethyl)pyridin-2-yl)oxy)methyl)piperidin-1-yl)-1H-pyrazolo[3,4-b]pyrazine (20 mg, 45.48%) as a colorless oil. ¹H NMR (400 MHZ, DMSO-d₆) δ 8.55-8.33 (m, 2H), 8.24-8.00 (m, 2H), 7.21-7.18 (m, 1H), 6.55-6.25 (m, 1H), 4.85 (s, 1H), 4.79-4.56 (m, 3H), 4.52-4.48 (m, 1H), 4.34-4.29 (m, 1H), 2.92-2.86 (m, 1H), 2.16-2.02 (m, 1H), 1.93-1.49 (m, 4H), 1.23-1.21 (m, 3H). MS m/z: 457.1 [M+H]⁺.

### 1-(2,2-Difluoroethyl)-6-((2R,5S)-2-methyl-5-(((2-(trifluoromethyl)pyridin-4-yl)oxy)methyl)piperidin-1-yl)-1H-pyrazolo[3,4-b]pyrazine (278)

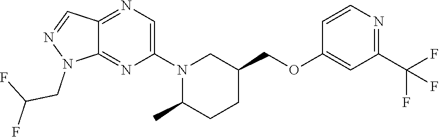 [SMILES: C[C@@H]1CC[C@H](COc2ccnc(C(F)(F)F)c2)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30 mg, 0.096 mmol, 1 equiv) and 4-fluoro-2-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((2-(trifluoromethyl)pyridin-4-yl)oxy)methyl)piperidin-1-yl)-1H-pyrazolo[3,4-b]pyrazine (20 mg, 45.48%) as a colorless oil. ¹H NMR (400 MHZ, DMSO-d₆) δ 8.60-8.59 (m, 1H), 8.49-8.46 (m, 1H), 8.13 (s, 1H), 7.53-7.51 (m, 1H), 7.35-7.33 (m, 1H), 6.63-6.26 (m, 1H), 4.91 (s, 1H), 4.72-4.64 (m, 2H), 4.60-4.57 (m, 1H), 4.26-4.12 (m, 2H), 2.92 (s, 1H), 2.07 (s, 1H), 1.81-1.74 (m, 3H), 1.65-1.60 (m, 1H), 1.23-1.21 (m, 3H). MS m/z: 457.1 [M+H]⁺.

### (3-(Phenoxymethyl)piperidin-1-yl)(4-(2-phenylthiazol-5-yl)tetrahydro-2H-pyran-4-yl)methanone (279)

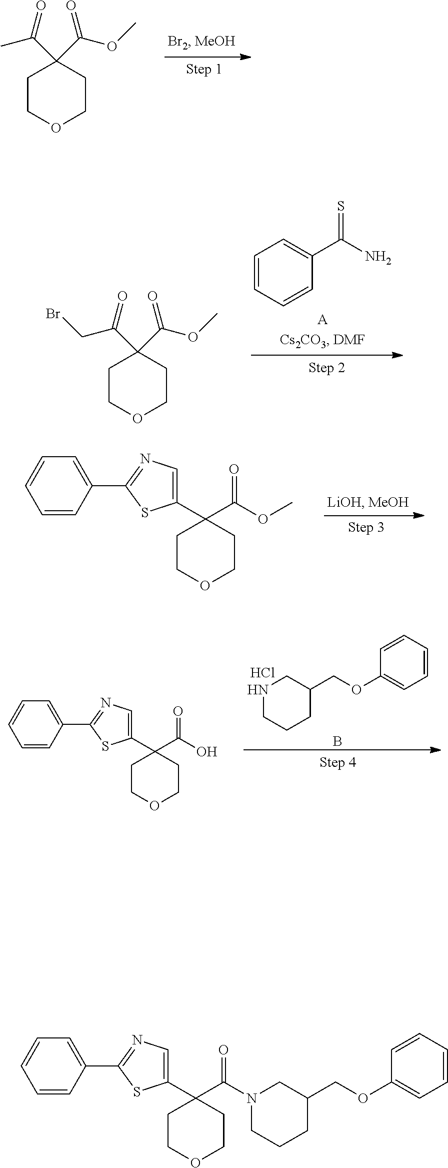

Step 1: methyl 4-(2-bromoacetyl)tetrahydro-2H-pyran-4-carboxylate: A solution of methyl 4-acetyltetrahydro-2H-pyran-4-carboxylate (500 mg, 2.68 mmol, 1 equiv) and Br₂ (643 mg, 4.02 mmol, 1.5 equiv) in MeOH (5 mL) at 0° C. The resulting mixture was stirred for 2 hours at 50° C. The resulting mixture was concentrated under vacuum. The residue was purified by flash chromatography with the following conditions: column, C18 silica gel; mobile phase, EA in PE, 0% to 100% gradient in 15 min; detector, UV 254 nm. This provided methyl 4-(2-bromoacetyl)tetrahydro-2H-pyran-4-carboxylate (400 mg, 56.2%) as a yellow oil. MS m/z: 265 [M+H]⁺.

Step 2: methyl 4-(2-phenylthiazol-5-yl)tetrahydro-2H-pyran-4-carboxylate: A solution of methyl 4-(2-bromoacetyl)tetrahydro-2H-pyran-4-carboxylate (200 mg, 0.754 mmol, 1 equiv) and thiobenzamide (155 mg, 1.13 mmol, 1.5 equiv) and Cs₂CO₃ (737 mg, 2.26 mmol, 3 equiv) in DMF (3 mL) was stirred for 2 hours at 100° C. The residue was purified by reverse flash chromatography with the following conditions: column, C18 silica gel; mobile phase, MeCN in water, 5% to 100% gradient in 15 min; detector, UV 254 nm. This provided methyl 4-(2-phenylthiazol-5-yl)tetrahydro-2H-pyran-4-carboxylate (80 mg, 34.9%) as a yellow oil. MS m/z: 304 [M+H]⁺.

Step 3: 4-(2-phenylthiazol-5-yl)tetrahydro-2H-pyran-4-carboxylic acid: A solution of methyl 4-(2-phenylthiazol-5-yl)tetrahydro-2H-pyran-4-carboxylate (80 mg, 0.264 mmol, 1 equiv) and NaOH (42.1 mg, 1.05 mmol, 4 equiv) in MeOH (1 mL) and H₂O (1 mL) was stirred for 2 hours at 50° C. The mixture was acidified to pH 5 with 1M HCl (aq.). The resulting mixture was extracted with EtOAc (3×20 mL). The combined organic layers were washed with brine (1×30 mL), dried over anhydrous Na₂SO₄. After filtration, the filtrate was concentrated under reduced pressure. This provided 4-(2-phenylthiazol-5-yl)tetrahydro-2H-pyran-4-carboxylic acid (50 mg, 65.5%) as a yellow oil. The crude product was used in the next step directly without further purification. MS m/z: 290 [M+H]⁺.

Step 4: (3-(phenoxymethyl)piperidin-1-yl)(4-(2-phenylthiazol-5-yl)tetrahydro-2H-pyran-4-yl)methanone: A solution of 4-(2-phenylthiazol-5-yl)tetrahydro-2H-pyran-4-carboxylic acid (50 mg, 0.173 mmol, 1 equiv) and 3-(phenoxymethyl)piperidine hydrochloride (39.35 mg, 0.173 mmol, 1 equiv) and HATU (65.71 mg, 0.173 mmol, 1 equiv) and DIEA (67.00 mg, 0.519 mmol, 3 equiv) in DMF (1 mL) was stirred for 2 h at room temperature. The residue was purified by reverse flash chromatography with the following conditions: column, C 18 silica gel; mobile phase, MeCN in water, 5% to 100% gradient in 15 min; detector, UV 254 nm. This provided (3-(phenoxymethyl)piperidin-1-yl)(4-(2-phenylthiazol-5-yl)tetrahydro-2H-pyran-4-yl)methanone (28 mg, 34.9%) as a white solid. ¹H NMR (400 MHZ, DMSO-d₆) δ 7.96-7.87 (m, 2H), 7.60 (s, 1H), 7.54-7.40 (m, 3H), 7.22 (s, 2H), 6.99-6.67 (m, 3H), 4.54-3.42 (m, 8H), 3.03-2.58 (m, 2H), 2.32-2.00 (m, 4H), 1.80-1.07 (m, 5H). MS m/z: 463.2 [M+H]⁺.

### (6S)-6-(3-((o-Tolyloxy)methyl)piperidine-1-carbonyl)-7,8-dihydropyrrolo[1,2-a]pyrimidin-4(6H)-one (280)

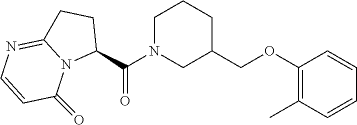 [SMILES: Cc1ccccc1OCC1CCCN(C(=O)[C@@H]2CCc3nccc(=O)n32)C1]

Followed General Procedure E using (S)-4-oxo-4,6,7,8-tetrahydropyrrolo[1,2-a]pyrimidine-6-carboxylic acid (60 mg, 0.333 mmol, 1.0 equiv) and 3-((o-tolyloxy)methyl)piperidine hydrochloride (80 mg, 0.333 mmol, 1.0 equiv). The crude product was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford (6S)-6-(3-((o-tolyloxy)methyl)piperidine-1-carbonyl)-7,8-dihydropyrrolo[1,2-a]pyrimidin-4(6H)-one (43 mg, 34.4%) as a white solid. ¹H NMR (400 MHz, DMSO-d₆) δ 7.85 (d, J=9.7 Hz, 1H), 7.22 (t, J=8.1 Hz, 2H), 6.95 (t, J=7.9 Hz, 1H), 6.87 (d, J=7.7 Hz, 1H), 6.32 (d, J=7.1 Hz, 1H), 5.79-5.43 (m, 2H), 4.49-3.73 (m, J=8.2. 1.4 Hz, 4H), 3.21-3.13 (m, 1H), 3.11-2.93 (m, J=7.1 Hz, 2H), 2.79-2.67 (m, 1H), 2.49-2.33 (m, 1H), 2.26-2.13 (m, J=7.0 Hz, 3H), 2.08-1.97 (m, J=11.9 Hz, 1H), 1.96-1.82 (m, 2H), 1.79-1.67 (m, 1H), 1.66-1.32 (m, 1H). MS m/z: 368.1 [M+H]⁺.

### 1-(Bicyclo[1.1.1]pentan-1-ylmethyl)-6-((3R,5S)-3-methyl-5-(((2-(trifluoromethyl)pyridin-3-yl)oxy)methyl)piperidin-1-yl)-1H-pyrazolo[3,4-b]pyrazine (281)

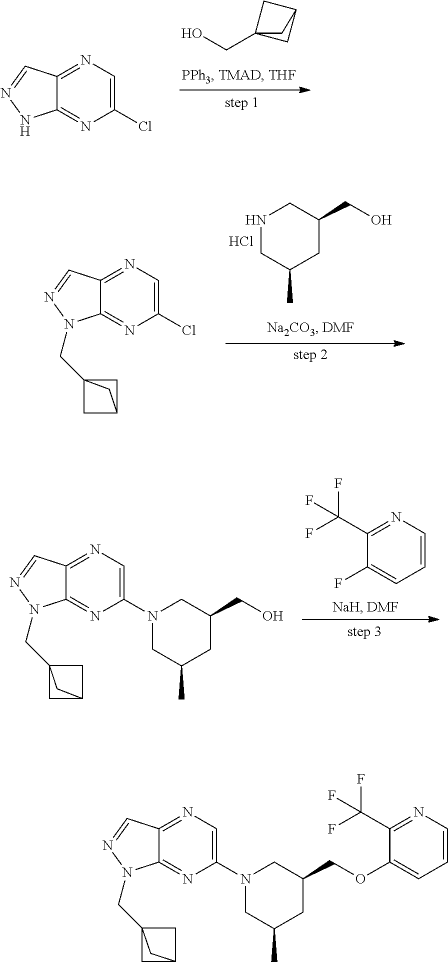

Step 1: 1-(bicyclo[1.1.1]pentan-1-ylmethyl)-6-chloro-1H-pyrazolo[3,4-b]pyrazine: Followed General Procedure A using 6-chloro-1H-pyrazolo[3,4-b]pyrazine (130 mg, 0.841 mmol, 1 equiv), bicyclo[1.1.1]pentan-1-ylmethanol (90.8 mg, 0.925 mmol, 1.1 equiv). The crude product was purified by silica gel column chromatography, eluted with EtOAc/PE (1/4) to afford 1-(bicyclo[1.1.1]pentan-1-ylmethyl)-6-chloro-1H-pyrazolo[3,4-b]pyrazine (100 mg, 50.7%) as a white solid. MS m/z: 235 [M+H]⁺.

Step 2: ((3S,5R)-1-(1-(bicyclo[1.1.1]pentan-1-ylmethyl)-1H-pyrazolo[3,4-b]pyrazin-6-yl)-5-methylpiperidin-3-yl)methanol: Followed General Procedure C using 1-(bicyclo[1.1.1]pentan-1-ylmethyl)-6-chloro-1H-pyrazolo[3,4-b]pyrazine (60 mg, 0.256 mmol, 1 equiv) and ((3S,5R)-5-methylpiperidin-3-yl)methanol hydrochloride (42.3 mg, 0.256 mmol, 1 equiv). The crude product was purified by reverse flash chromatography with the following conditions: column, C18 silica gel; mobile phase, ACN in water, 10% to 50% gradient in 10 min; detector, UV 254 nm. This provided ((3S,5R)-1-(1-(bicyclo[1.1.1]pentan-1-ylmethyl)-1H-pyrazolo[3,4-b]pyrazin-6-yl)-5-methylpiperidin-3-yl)methanol (60 mg, 71.6%) as a white solid. MS m/z: 328 [M+H]⁺.

Step 3: 1-(bicyclo[1.1.1]pentan-1-ylmethyl)-6-((3R,5S)-3-methyl-5-(((2-(trifluoromethyl)pyridin-3-yl)oxy)methyl)piperidin-1-yl)-1H-pyrazolo[3,4-b]pyrazine: Followed General Procedure D using ((3S,5R)-1-(1-(bicyclo[1.1.1]pentan-1-ylmethyl)-1H-pyrazolo[3,4-b]pyrazin-6-yl)-5-methylpiperidin-3-yl)methanol (60 mg, 0.183 mmol, 1 equiv) and 3-fluoro-2-(trifluoromethyl)pyridine (45.3 mg, 0.274 mmol, 1.5 equiv). The crude product was purified by reverse flash chromatography with the following conditions: column, C18 silica gel; mobile phase, MeCN in water, 0% to 100% gradient in 30 min; detector, UV 254 nm. This provided 1-(bicyclo[1.1.1]pentan-1-ylmethyl)-6-((3R,5S)-3-methyl-5-(((2-(trifluoromethyl)pyridin-3-yl)oxy)methyl)piperidin-1-yl)-1H-pyrazolo[3,4-b]pyrazine (33.4 mg, 38.4%) as a white solid. ¹H NMR (400 MHZ, DMSO-d₆) δ 8.41 (s, 1H), 8.31-8.25 (m, 1H), 8.01 (s, 1H), 7.84 (d, J=8.5 Hz, 1H), 7.74-7.68 (m, 1H), 4.91 (d, J=12.8 Hz, 1H), 4.54-4.45 (m, 1H), 4.32-4.20 (m, 3H), 4.06-3.98 (m, 1H), 2.70-2.62 (m, 1H), 2.61-2.54 (m, 1H), 2.41 (s, 1H), 2.17-2.06 (m, 1H), 1.89 (d, J=12.7 Hz, 1H), 1.77-1.67 (m, 1H), 1.62 (s, 6H), 1.12-1.03 (m, 1H), 0.97 (d, J=6.5 Hz, 3H). MS m/z: 473.2 [M+H]⁺.

### 1-(2,2-Difluoroethyl)-6-((2R,5S)-2-methyl-5-(((3-(trifluoromethyl)pyridin-4-yl)oxy)methyl)piperidin-1-yl)-1H-pyrazolo[3,4-b]pyrazine (282)

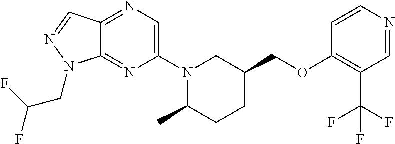 [SMILES: C[C@@H]1CC[C@H](COc2ccncc2C(F)(F)F)CN1c1cnc2cnn(CC(F)F)c2n1]

Followed General Procedure D using ((3S,6R)-1-(1-(2,2-difluoroethyl)-1H-pyrazolo[3,4-b]pyrazin-6-yl)-6-methylpiperidin-3-yl)methanol (30 mg, 0.096 mmol, 1 equiv) and 4-fluoro-3-(trifluoromethyl)pyridine (23.8 mg, 0.144 mmol, 1.5 equiv). The crude product was purified by reverse flash chromatography with the following conditions: column, silica gel; mobile phase, MeCN in water, 0% to 100% gradient in 30 min; detector, UV 254 nm. This provided 1-(2,2-difluoroethyl)-6-((2R,5S)-2-methyl-5-(((3-(trifluoromethyl)pyridin-4-yl)oxy)methyl)piperidin-1-yl)-1H-pyrazolo[3,4-b]pyrazine (13 mg, 29.5%) as a white solid. LCMS (ES, m/z): 457.1[M+H]⁺. ¹H NMR (400 MHZ, DMSO-d₆) δ 8.73-8.72 (m, 2H), 8.42 (s, 1H), 8.12 (s, 1H), 7.39-7.38 (m, 1H), 6.55-6.26 (m, 1H), 4.85 (s, 1H), 4.79-4.58 (m, 3H), 4.36-4.32 (m, 1H), 4.21-4.16 (m, 1H), 2.96-2.86 (m 1H), 2.09-2.06 (m, 1H), 1.86-1.80 (m, 1H), 1.78-1.62 (m, 3H), 1.23-1.21 (m, 3H).

### (S)-1-((2-Oxaspiro[3.3]heptan-6-yl)methyl)-6-(3-(((2-(trifluoromethyl)pyridin-3-yl)oxy) methyl) piperidin-1-yl)-1H-pyrazolo[3,4-b]pyrazine (283)

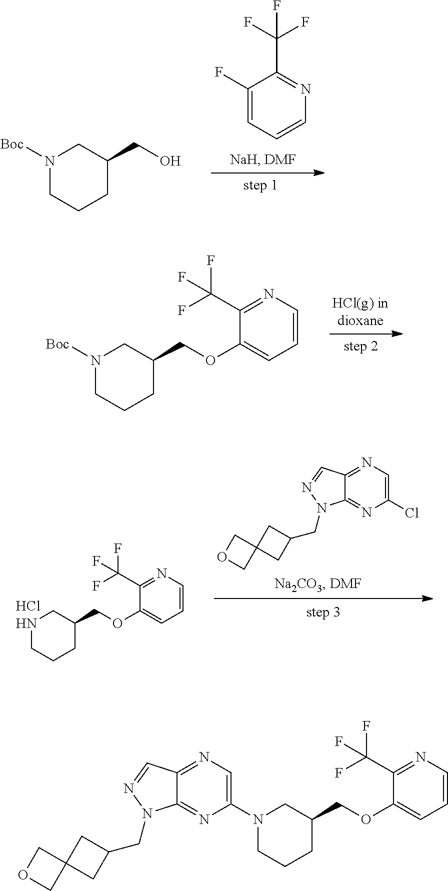

Step 1: tert-butyl (S)-3-(((2-(trifluoromethyl)pyridin-3-yl)oxy)methyl)piperidine-1-carboxylate: Followed General Procedure D using tert-butyl tert-butyl (S)-3-(hydroxymethyl)piperidine-1-carboxylate (1.00 g, 4.64 mmol, 1.00 equiv) and 3-fluoro-2-(trifluoromethyl)pyridine (0.770 g, 4.64 mmol, 1.00 equiv). The crude product was purified by silica gel column chromatography, eluted with PE/EtOAc (0% to 100% gradient in 20 min) to afford tert-butyl (S)-3-(((2-(trifluoromethyl)pyridin-3-yl)oxy)methyl)piperidine-1-carboxylate (1.63 g, 97.7%) as a yellow oil. MS m/z: 361 [M+H]⁺.

Step 2: (S)-3-(piperidin-3-ylmethoxy)-2-(trifluoromethyl)pyridine hydrochloride: Followed General Procedure B using tert-butyl (S)-3-(((2-(trifluoromethyl)pyridin-3-yl)oxy)methyl)piperidine-1-carboxylate (723 mg, 2.00 mmol, 1.00 equiv) to afford the crude product (S)-3-(piperidin-3-ylmethoxy)-2-(trifluoromethyl)pyridine hydrochloride (752.6 mg). MS m/z: 261 [M+H]⁺.

Step 3: (S)-1-((2-oxaspiro[3.3]heptan-6-yl)methyl)-6-(3-(((2-(trifluoromethyl)pyridin-3-yl)oxy) methyl) piperidin-1-yl)-1H-pyrazolo[3,4-b]pyrazine: Followed General Procedure C using (S)-3-(piperidin-3-ylmethoxy)-2-(trifluoromethyl)pyridine hydrochloride (134 mg, 0.454 mmol, 1.10 equiv) and 1-((2-oxaspiro[3.3]heptan-6-yl)methyl)-6-chloro-1H-pyrazolo[3,4-b]pyrazine (109 mg, 0.413 mmol, 1.00 equiv). The crude product was purified by reverse flash chromatography with the following conditions: Column: Xselect CSH C18 OBD Column 30*150 mm 5 um, n; Mobile Phase A: Water(0.1% FA), Mobile Phase B: ACN; Flow rate: 60 mL/min; Gradient: 40% B to 85% B in 15 min, 85% B; Wave Length: 254/220 nm; RT1(min): 6.48 to afford (S)-1-((2-oxaspiro[3.3]heptan-6-yl)methyl)-6-(3-(((2-(trifluoromethyl)pyridin-3-yl)oxy)methyl)piperidin-1-yl)-1H-pyrazolo[3,4-b]pyrazine (46.5 mg, 22.9%) as a light yellow solid. ¹H NMR (400 MHZ, DMSO-d₆) δ 8.38 (s, 1H), 8.28-8.27 (m, 1H), 8.01 (s, 1H), 7.82 (d, J=8.5 Hz, 1H), 7.71-7.68 (m, 1H), 4.71-4.66 (m, 1H), 4.51 (s, 2H), 4.39 (s, 3H), 4.24-4.21 (m, 1H), 4.16 (d, J=7.2 Hz, 2H), 4.09-4.04 (m, 1H), 3.17-3.11 (m, 1H), 3.01-2.95 (m, 1H), 2.63-2.53 (m, 1H), 2.28-2.18 (m, 2H), 2.17-2.06 (m, 1H), 2.04-1.95 (m, 2H), 1.94-1.87 (m, 1H), 1.84-1.79 (m, 1H), 1.65-1.39 (m, 2H). MS m/z: 489.2 [M+H]⁺.

### 3-[1-(2,2-Difluoroethyl)-1H-Pyrazolo[3,4-b]Pyrazin-6-Yl]-1-({[6-(Trifluoromethyl)Pyridin-2-Yl]Oxy}Methyl)-3-Azabicyclo[3.1.0]Hexane (284)

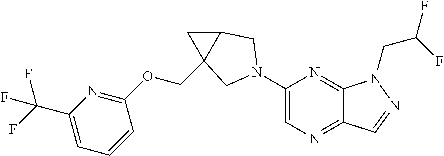 [SMILES: FC(F)Cn1ncc2ncc(N3CC4CC4(COc4cccc(C(F)(F)F)n4)C3)nc21]

Step 1: tert-butyl 1-({[6-(trifluoromethyl)pyridin-2-yl]oxy}methyl)-3-azabicyclo[3.1.0]hexane-3-carboxylate: Followed General Procedure D using 2-bromo-6-(trifluoromethyl)pyridine (53 mg, 0.23 mmol, 1.00 equiv) and tert-butyl 1-(hydroxymethyl)-3-azabicyclo[3.1.0]hexane-3-carboxylate (50 mg, 0.23 mmol, 1.0 equiv) to afford tert-butyl 1-({[6-(trifluoromethyl)pyridin-2-yl]oxy}methyl)-3-azabicyclo[3.1.0]hexane-3-carboxylate (60 mg, 71%) as a colorless oil. MS m/z: 359 [M+H]⁺.

Step 2: 1-({[6-(trifluoromethyl)pyridin-2-yl]oxy}methyl)-3-azabicyclo[3.1.0]hexane hydrochloride: Followed General Procedure B using tert-butyl 1-({[6-(trifluoromethyl)pyridin-2-yl]oxy}methyl)-3-azabicyclo[3.1.0]hexane-3-carboxylate (60 mg, 0.167 mmol, 1.00 equiv) to afford the crude product 1-({[6-(trifluoromethyl)pyridin-2-yl]oxy}methyl)-3-azabicyclo[3.1.0]hexane hydrochloride (50 mg). MS m/z: 259 [M+H]⁺.

Step 3: 3-[1-(2,2-difluoroethyl)-1H-pyrazolo[3,4-b]pyrazin-6-yl]-1-({[6-(trifluoromethyl)pyridin-2-yl]oxy}methyl)-3-azabicyclo[3.1.0]hexane: Followed General Procedure C using 1-({[6-(trifluoromethyl)pyridin-2-yl]oxy}methyl)-3-azabicyclo[3.1.0]hexane hydrochloride (32 mg, 0.11 mmol, 1.00 equiv) and 6-chloro-1-(2,2-difluoroethyl)-1H-pyrazolo[3,4-b]pyrazine (23 mg, 0.11 mmol, 1.00 equiv). The crude product was purified by reverse flash chromatography with the following conditions: column, C18 silica gel; mobile phase, ACN in water, 10% to 50% gradient in 30 min; detector, UV 254/220 nm to afford 3-[1-(2,2-difluoroethyl)-1H-pyrazolo[3,4-b]pyrazin-6-yl]-1-({[6-(trifluoromethyl)pyridin-2-yl]oxy}methyl)-3-azabicyclo[3.1.0]hexane (32 mg, 68%) as a colorless oil. ¹H NMR (500 MHZ, DMSO-d₆) δ 8.13 (s, 1H), 8.07 (s, 1H), 7.99 (ddd, J=8.5, 7.5, 0.9 Hz, 1H), 7.50 (d, J=7.3 Hz, 1H), 7.21 (d, J=8.4 Hz, 1H), 6.43 (tt, J=54.9, 3.9 Hz, 1H), 4.73-4.58 (m, 3H), 4.40 (d, J=11.5 Hz, 1H), 4.00 (d, J=10.8 Hz, 1H), 3.88 (d, J=10.7 Hz, 1H), 3.67 (dd, J=32.3, 9.3 Hz, 2H), 1.95-1.82 (m, 1H), 1.09 (dd, J=8.2, 4.8 Hz, 1H), 0.61 (t, J=4.6 Hz, 1H). MS m/z: 441.2 [M+H]⁺.

### 3-[1-(2,2-Difluoroethyl)-1H-pyrazolo[3,4-b]pyrazin-6-yl]-1-({[4-(trifluoromethyl)pyridin-2-yl]oxy}methyl)-3-azabicyclo[3.1.0]hexane (285)

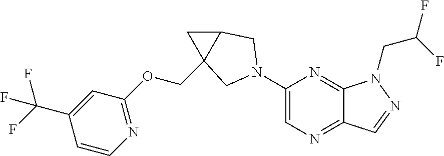 [SMILES: FC(F)Cn1ncc2ncc(N3CC4CC4(COc4cc(C(F)(F)F)ccn4)C3)nc21]

Step 1: tert-butyl 1-({[4-(trifluoromethyl)pyridin-2-yl]oxy}methyl)-3-azabicyclo[3.1.0]hexane-3-carboxylate: Followed General Procedure D using 2-bromo-4-(trifluoromethyl)pyridine (53 mg, 0.23 mmol, 1.00 equiv) and tert-butyl 1-(hydroxymethyl)-3-azabicyclo[3.1.0]hexane-3-carboxylate (50 mg, 0.23 mmol, 1.0 equiv) to afford tert-butyl 1-({[4-(trifluoromethyl)pyridin-2-yl]oxy}methyl)-3-azabicyclo[3.1.0]hexane-3-carboxylate (55 mg, 65%) as a colorless oil. MS m/z: 359 [M+H]⁺.

Step 2: 1-({[4-(trifluoromethyl)pyridin-2-yl]oxy}methyl)-3-azabicyclo[3.1.0]hexane hydrochloride: Followed General Procedure B using tert-butyl 1-({[4-(trifluoromethyl)pyridin-2-yl]oxy}methyl)-3-azabicyclo[3.1.0]hexane-3-carboxylate (55 mg, 0.153 mmol, 1.00 equiv) to afford the crude product 1-({[4-(trifluoromethyl)pyridin-2-yl]oxy}methyl)-3-azabicyclo[3.1.0]hexane hydrochloride (50 mg). MS m/z: 259 [M+H]⁺.

Step 3: 3-[1-(2,2-difluoroethyl)-1H-pyrazolo[3,4-b]pyrazin-6-yl]-1-({[4-(trifluoromethyl)pyridin-2-yl]oxy}methyl)-3-azabicyclo[3.1.0]hexane: Followed General Procedure C using 1 1-({[4-(trifluoromethyl)pyridin-2-yl]oxy}methyl)-3-azabicyclo[3.1.0]hexane (35 mg, 0.12 mmol, 1.00 equiv) and 6-chloro-1-(2,2-difluoroethyl)-1H-pyrazolo[3,4-b]pyrazine (26 mg, 0.12 mmol, 1.00 equiv). The crude product was purified by reverse flash chromatography with the following conditions: column, C18 silica gel; mobile phase, ACN in water, 10% to 50% gradient in 30 min; detector, UV 254/220 nm to afford 3-[1-(2,2-difluoroethyl)-1H-pyrazolo[3,4-b]pyrazin-6-yl]-1-({[4-(trifluoromethyl)pyridin-2-yl]oxy}methyl)-3-azabicyclo[3.1.0]hexane (20 mg, 39%) as a colorless oil. ¹H NMR (500 MHZ, DMSO-d₆) δ 8.44 (d, J=5.3 Hz, 1H), 8.13 (s, 1H), 8.09 (s, 1H), 7.37-7.30 (m, 1H), 7.28 (s, 1H), 6.44 (tt, J=54.9, 3.9 Hz, 2H), 4.68 (td, J=15.0, 3.8 Hz, 2H), 4.61 (d, J=11.4 Hz, 1H), 4.49 (d, J=11.4 Hz, 1H), 4.02 (d, J=10.7 Hz, 1H), 3.89 (d, J=10.7 Hz, 1H), 3.67 (dd, J=23.8, 10.6 Hz, 2H), 1.91-1.83 (m, 1H), 1.08 (dd, J=8.2, 4.8 Hz, 1H), 0.62 (t, J=4.6 Hz, 1H). MS m/z: 441.2 [M+H]⁺.

### 6-((1R,5S,6r)-6-((3,5-Difluorophenoxy)methyl)-3-azabicyclo[3.1.0]hexan-3-yl)-2-(5-fluoropyridin-2-yl)-1-methyl-1H-imidazo[4,5-b]pyrazine (286)

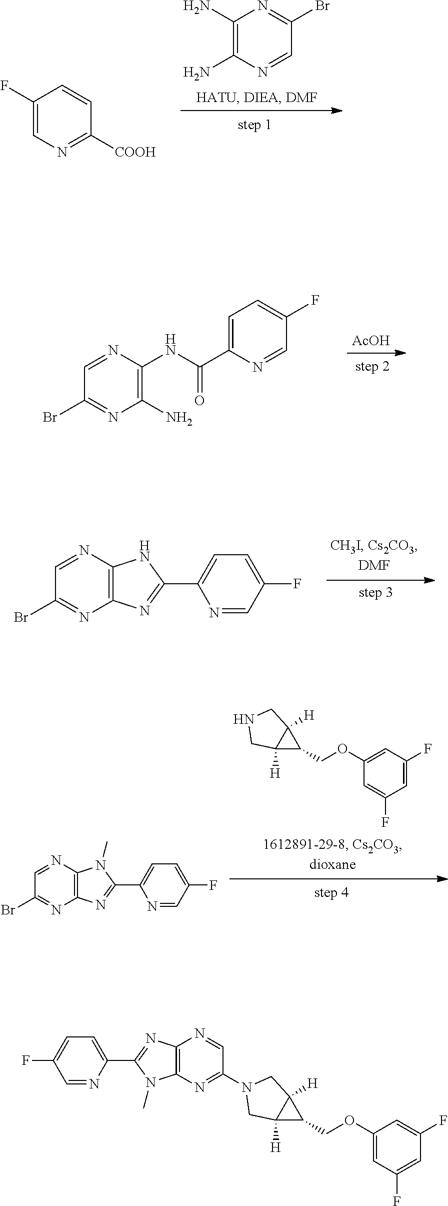

Step 1: N-(3-amino-5-bromopyrazin-2-yl)-5-fluoropicolinamide: A solution of 5-fluoropicolinic acid (1 g, 7.09 mmol, 1 equiv), 5-bromopyrazine-2,3-diamine (1.34 g, 7.09 mmol, 1 equiv), HATU (4.04 g, 10.6 mmol, 1.5 equiv) and DIPEA (2.75 g, 21.3 mmol, 3 equiv) in DMF (10 mL) was stirred for overnight at 70° C. The resulting mixture was diluted with EtOAc (80 mL). The combined organic layers were washed with water (3×40 mL), dried over anhydrous Na₂SO₄. After filtration, the filtrate was concentrated under reduced pressure. The residue was purified by silica gel column chromatography, eluted with CH₂Cl₂/MeOH (20:1) to afford N-(3-amino-5-bromopyrazin-2-yl)-5-fluoropyridine-2-carboxamide (800 mg, 36.2%). MS m/z: 312 [M+H]⁺.

Step 2: 5-bromo-2-(5-fluoropyridin-2-yl)-1H-imidazo[4,5-b]pyrazine: A solution of N-(3-amino-5-bromopyrazin-2-yl)-5-fluoropyridine-2-carboxamide (800 mg, 2.56 mmol, 1 equiv) in CH₃COOH (5 mL) was stirred for 2 days at 100° C. The resulting mixture was concentrated under vacuum. The residue was dissolved in CH₂Cl₂ (30 mL). The resulting mixture was washed with 2×10 mL of sat. NaHCO₃(aq.). The resulting mixture was concentrated under vacuum. The residue was purified by reverse flash chromatography with the following conditions: column, C18 silica gel; mobile phase, MeCN in Water (0.1% NH₃·H₂O), 10% to 100% gradient in 30 min; detector, UV 254 nm. This provided 5-bromo-2-(5-fluoropyridin-2-yl)-1H-imidazo[4,5-b]pyrazine (100 mg, 13.3%). MS m/z: 294 [M+H]⁺.

Step 3: 5-bromo-2-(5-fluoropyridin-2-yl)-1H-imidazo[4,5-b]pyrazine: A solution of 5-bromo-2-(5-fluoropyridin-2-yl)-1H-imidazo[4,5-b]pyrazine (100 mg, 0.34 mmol, 1 equiv), MeI (72.4 mg, 0.51 mmol, 1.5 equiv) and Cs₂CO₃ (332 mg, 1.02 mmol, 3 equiv) in DMF (1.5 mL) was stirred for overnight at room temperature. The resulting mixture was extracted with EtOAc (30 mL). The combined organic layers were washed with water (3×15 mL), dried over anhydrous Na₂SO₄. After filtration, the filtrate was concentrated under reduced pressure. The residue was purified by silica gel column chromatography, eluted with PE/EA (1:1) to afford 5-bromo-2-(5-fluoropyridin-2-yl)-1H-imidazo[4,5-b]pyrazine (50 mg, 47.7%). MS m/z: 308 [M+H]⁺.

Step 4. 6-((1R,5S,6r)-6-((3,5-difluorophenoxy)methyl)-3-azabicyclo[3.1.0]hexan-3-yl)-2-(5-fluoropyridin-2-yl)-1-methyl-1H-imidazo[4,5-b]pyrazine: A solution of 5-bromo-2-(5-fluoropyridin-2-yl)-1H-imidazo[4,5-b]pyrazine (30 mg, 0.1 mmol, 1 equiv), (1R,5S,6r)-6-((3,5-difluorophenoxy)methyl)-3-azabicyclo[3.1.0]hexane (28 mg, 0.1 mmol, 1.1 equiv), Cs₂CO₃ (63 mg, 0.2 mmol, 2 equiv) and Pd-PEPPSI-IPentCl 2-methylpyridine (o-picoline (8.19 mg, 0.01 mmol, 0.1 equiv) in dioxane (2 mL) was stirred for overnight at 80° C. under argon atmosphere. The residue was purified by reverse flash chromatography with the following conditions: column, C18 silica gel; mobile phase, MeCN in water, 10% to 100% gradient in 30 min; detector, UV 254 nm. This provided 6-((1R,5S,6r)-6-((3,5-difluorophenoxy)methyl)-3-azabicyclo[3.1.0]hexan-3-yl)-2-(5-fluoropyridin-2-yl)-1-methyl-1H-imidazo[4,5-b]pyrazine (6 mg, 12%) as a yellow solid. ¹H NMR (400 MHZ, CDCl₃) δ 8.51 (t, J=4.8 Hz, 2H), 7.86 (s, 1H), 7.55 (td, J=8.5, 2.8 Hz, 1H), 6.47-6.36 (m, 3H), 4.20 (s, 3H), 3.94 (dd, J=29.1, 8.6 Hz, 4H), 3.67-3.56 (m, 2H), 1.85-1.81 (m, 1H), 1.31-1.23 (m, 2H). MS m/z: 453.0 [M+H]⁺.

### 5-((1R,5S,6r)-6-((3,5-Difluorophenoxy)methyl)-3-azabicyclo[3.1.0]hexan-3-yl)-2-(5-fluoropyridin-2-yl)-1-methyl-1H-imidazo[4,5-b]pyrazine (287)

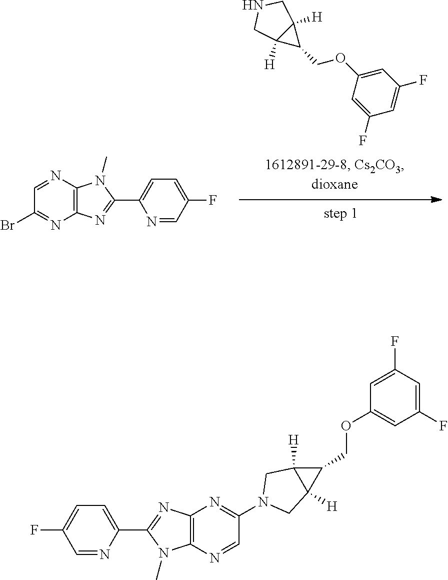 [SMILES: Fc1cc(F)cc(OC[C@H]2[C@@H]3CNC[C@@H]32)c1 ; Cn1c(-c2ccc(F)cn2)nc2nc(Br)cnc21 ; Cn1c(-c2ccc(F)cn2)nc2nc(N3C[C@@H]4[C@@H](COc5cc(F)cc(F)c5)[C@@H]4C3)cnc21]

A solution of 5-bromo-2-(5-fluoropyridin-2-yl)-1-methyl-1H-imidazo[4,5-b]pyrazine (30 mg, 0.1 mmol, 1 equiv), (1R,5S,6r)-6-((3,5-difluorophenoxy)methyl)-3-azabicyclo[3.1.0]hexane (27 mg, 0.11 mmol, 1.1 equiv), Cs₂CO₃ (95.17 mg, 0.291 mmol, 3 equiv) and Pd-PEPPSI-IPentCl 2-methylpyridine (o-picoline) (8.19 mg, 0.01 mmol, 0.1 equiv) in dioxane (2 mL) was stirred for overnight at 80° C. under argon atmosphere. The reaction mixture was diluted by EtOAc (20 mL), washed by water (2×10 mL) and brine (1×10 mL), dried over anhydrous sodium sulfate. After filtration, the filtrate was concentrated. The residue was purified by silica gel column chromatography, eluted with EtOAc/PE=3/2, to afford impure product. This was purified by reverse flash chromatography with the following conditions: column, C18 silica gel; mobile phase, MeCN in water, 10% to 100% gradient in 30 min; detector, UV 254 nm. This provided 5-((1R,5S,6r)-6-((3,5-difluorophenoxy)methyl)-3-azabicyclo[3.1.0]hexan-3-yl)-2-(5-fluoropyridin-2-yl)-1-methyl-1H-imidazo[4,5-b]pyrazine (4.6 mg, 9.40%) as a yellow solid. ¹H NMR (400 MHZ, CDCl₃) δ 8.60-8.52 (m, 2H), 7.70 (s, 1H), 7.62-7.50 (m, 1H), 6.46-6.37 (m, 3H), 4.29 (s, 3H), 3.93 (dd, J=28.0, 8.5 Hz, 4H), 3.66-3.57 (m, 3H), 1.87-1.78 (m, 2H), 1.32-1.20 (m, 1H). MS m/z: 453.20 [M+H]⁺.

### 1,3-Diethyl-5-((3S,5R)-3-methyl-5-(((2-(trifluoromethyl)pyridin-3-yl)oxy)methyl)piperidin-1-yl)-1,3-dihydro-2H-imidazo[4,5-b]pyrazin-2-one (288a); 1,3-Diethyl-5-((3S,5S)-3-methyl-5-(((2-(trifluoromethyl)pyridin-3-yl)oxy)methyl)piperidin-1-yl)-1,3-dihydro-2H-imidazo[4,5-b]pyrazin-2-one (288b)

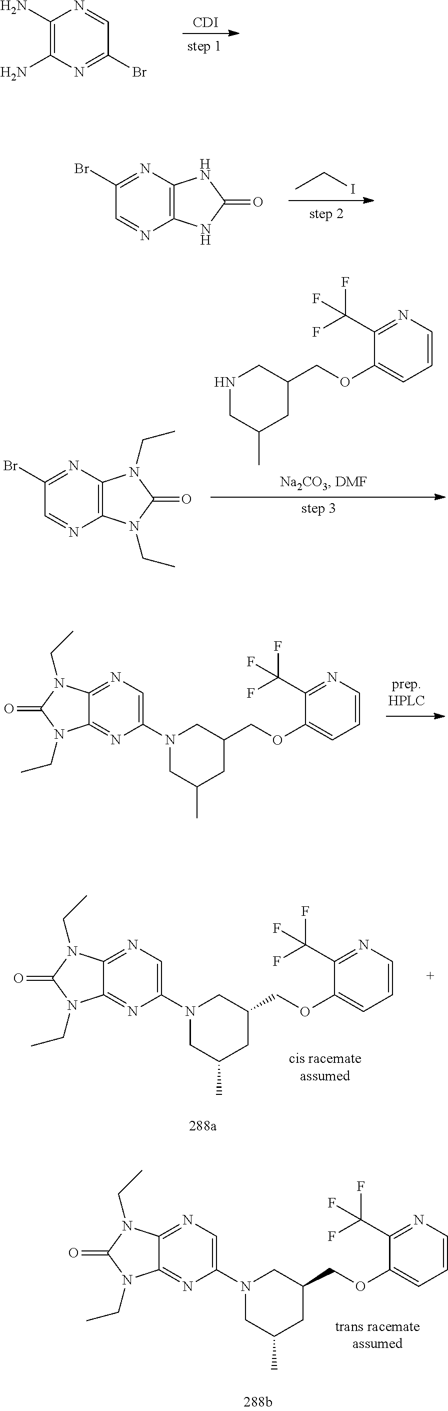

Step 1: 5-bromo-1,3-dihydro-2H-imidazo[4,5-b]pyrazin-2-one: To a stirred solution of 5-bromopyrazine-2,3-diamine (1 g, 5.29 mmol, 1 equiv) and CDI (0.94 g, 5.82 mmol, 1.1 equiv) in ACN (4.00 mL) was added Na₂CO₃ (1.68 g, 15.9 mmol, 3 equiv). The resulting mixture was stirred for overnight at 80° C. The residue was purified by reverse flash chromatography with the following conditions (column, C18 gel; mobile phase, B phase: MeCN, A phase: water; 0% to 100% B gradient in 20 min; detector: UV 254/220 nm). This provided 5-bromo-1,3-dihydro-2H-imidazo[4,5-b]pyrazin-2-one (300 mg, 26.4%) as a white solid. MS m/z: 215 [M+H]⁺.

Step 2: 5-bromo-1,3-diethyl-1,3-dihydro-2H-imidazo[4,5-b]pyrazin-2-one: To a stirred solution of 5-bromo-1,3-dihydro-2H-imidazo[4,5-b]pyrazin-2-one (300 mg, 1.4 mmol, 1 equiv) and iodoethane (653 mg, 4.19 mmol, 3 equiv) in DMF (2.00 mL) was added Cs₂CO₃ (909 mg, 2.79 mmol, 2 equiv). The resulting mixture was stirred for overnight at room temperature. The residue was purified by reverse flash chromatography with the following conditions (column, C18 gel; mobile phase, B phase: MeCN, A phase: water; 0% to 100% B gradient in 20 min; detector: UV 254/220 nm). This provided 5-bromo-1,3-diethyl-1,3-dihydro-2H-imidazo[4,5-b]pyrazin-2-one (180 mg, 47.6%) as a white solid. MS m/z: 271 [M+H]⁺.

Step 3: 1,3-diethyl-5-(3-methyl-5-(((2-(trifluoromethyl)pyridin-3-yl)oxy)methyl)pi-peridin-1-yl)-1,3-dihydro-2H-imidazo[4,5-b]pyrazin-2-one: A solution of 5-bromo-1,3-diethyl-1,3-dihydro-2H-imidazo[4,5-b]pyrazin-2-one (180 mg, 0.664 mmol, 1 equiv) and 3-((5-methylpiperidin-3-yl)methoxy)-2-(trifluoromethyl)pyridine (200 mg, 0.730 mmol, 1.1 equiv) and Na₂CO₃ (211 mg, 1.99 mmol, 3 equiv) in DMF (2.0 mL) was stirred for 2 hours at 100° C. The residue was purified by reverse flash chromatography with the following conditions: column, silica gel; mobile phase, MeCN in water, 0% to 100% gradient in 20 min; detector, UV 254 nm. This provided 1,3-diethyl-5-(3-methyl-5-(((2-(trifluoromethyl)pyridin-3-yl)oxy)methyl)piperidin-1-yl)-1,3-dihydro-2H-imidazo[4,5-b]pyrazin-2-one as a white solid. This product was further purified by prep. HPLC with the following conditions: column: XBridge Prep F-Phenyl OBD Column, 19*100 mm, 5 um; Mobile Phase A: Water(0.1% FA), Mobile Phase B: ACN; Flow rate: 25 mL/min; Gradient: 45% B to 71% B in 7 min, 71% B; Wave Length: 254/220 nm; RT1(min): 6.35. This provided 1,3-diethyl-5-((3S,5R)-3-methyl-5-(((2-(trifluoromethyl)pyridin-3-yl)oxy)methyl)piperidin-1-yl)-1,3-dihydro-2H-imidazo[4,5-b]pyrazin-2-one (288a, 11 mg, 10.5%) and 1,3-diethyl-5-((3S,5S)-3-methyl-5-(((2-(trifluoromethyl)pyridin-3-yl)oxy)methyl)piperidin-1-yl)-1,3-dihydro-2H-imidazo[4,5-b]pyrazin-2-one (288b, 49 mg, 46.7%) as white solids.

288a: ¹H NMR (300 MHZ, DMSO-d₆) δ 8.24 (d, J=4.4 Hz, 1H), 7.86-7.75 (m, 1H), 7.73-7.64 (m, 1H), 7.58 (s, 1H), 4.35-4.04 (m, 4H), 3.92-3.76 (m, 4H), 2.81 (t. J=12.3 Hz, 1H), 1.99-1.78 (m, 1H), 1.75-1.49 (m, 3H), 1.31-1.21 (m, 6H), 1.05-0.94 (m, 3H). MS m/z: 465.2 [M+H]⁺.

288b: ¹H NMR (300 MHz, DMSO-d₆) δ 8.32-8.14 (m, 1H), 7.91-7.81 (m, 1H), 7.72-7.65 (m, 1H), 7.56 (s, 1H), 4.20-3.99 (m, 3H), 3.95-3.75 (m, 5H), 3.11-3.03 (m, 1H), 2.99-2.84 (m, 1H), 2.27-2.10 (m, 2H), 1.72-1.59 (m, 2H), 1.33-1.14 (m, 6H), 0.99-0.89 (m, 3H). MS m/z: 465.2 [M+H]⁺.

### 1-(2,2-Difluoroethyl)-N-methyl-N-((1-(3-(trifluoromethyl)pyridin-2-yl)piperidin-3-yl)methyl)-1H-pyrazolo[3,4-b]pyrazin-6-amine (289)

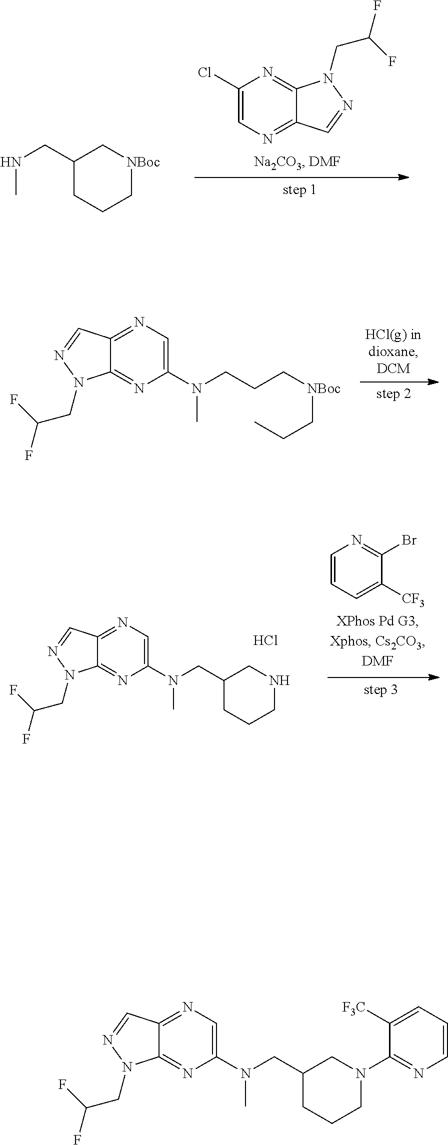

Step 1: tert-butyl 3-(((1-(2,2-difluoroethyl)-1H-pyrazolo[3,4-b]pyrazin-6-yl)(methyl) amino)methyl)piperidine-1-carboxylate: Followed General Procedure C using tert-butyl 3-((methylamino)methyl)piperidine-1-carboxylate (83.6 mg, 0.366 mmol, 1 equiv) and 6-chloro-1-(2,2-difluoroethyl)pyrazolo[3,4-b]pyrazine (80 mg, 0.366 mmol, 1.00 equiv). The crude product was purified by silica gel column chromatography, eluted with PE/EA (1:1) to afford tert-butyl tert-butyl 3-(((1-(2,2-difluoroethyl)-1H-pyrazolo[3,4-b]pyrazin-6-yl)(methyl)amino)methyl)piperidine-1-carboxylate (100 mg, 59.9%) as a white oil. MS m/z: 411 [M+H]⁺.

Step 2: 1-(2,2-difluoroethyl)-N-methyl-N-(piperidin-3-ylmethyl)-1H-pyrazolo[3,4-b]pyrazin-6-amine hydrochloride: Followed General Procedure B using tert-butyl tert-butyl 3-(((1-(2,2-difluoroethyl)-1H-pyrazolo[3,4-b]pyrazin-6-yl)(methyl)amino)methyl)piperidine-1-carboxylate (100 mg, 0.244 mmol, 1 equiv) to afford the crude product 1-(2,2-difluoroethyl)-N-methyl-N-(piperidin-3-ylmethyl)-1H-pyrazolo[3,4-b]pyrazin-6-amine hydrochloride. MS m/z: 311 [M+H]⁺.

Step 3: 1-(2,2-difluoroethyl)-N-methyl-N-((1-(3-(trifluoromethyl)pyridin-2-yl)piperidin-3-yl)methyl)-1H-pyrazolo[3,4-b]pyrazin-6-amine: To a stirred mixture of 2-bromo-3-(trifluoromethyl)pyridine (87.4 mg, 0.386 mmol, 1.2 equiv) and Cs₂CO₃ (315 mg, 0.966 mmol, 3 equiv) in DMF (2 mL) were added 1-(2,2-difluoroethyl)-N-methyl-N-(piperidin-3-ylmethyl)pyrazolo[3,4-b]pyrazin-6-amine (100 mg, 0.322 mmol, 1 equiv) and XPhos Pd G3 (27.3 mg, 0.0320 mmol, 0.1 equiv) and X-Phos (15.4 mg, 0.0320 mmol, 0.1 equiv) in portions at room temperature. The resulting mixture was stirred for 3 h at 100° C. The mixture was allowed to cool down to room temperature. The reaction mixture was diluted by EtOAc (20 mL), washed by water (2×10 mL) and brine (1×15 mL), dried over anhydrous sodium sulfate. After filtration, the filtrate was concentrated. The residue was purified by silica gel column chromatography, eluted with EtOAc/PE=3/2, to afford impure product. This was further purified by reverse flash chromatography with the following conditions: column, C18 silica gel; mobile phase, MeCN in water, 5% to 100% gradient in 20 min; detector, UV 254 nm. This provided 1-(2,2-difluoroethyl)-N-methyl-N-({1-[3-(trifluoromethyl)pyridin-2-yl]piperidin-3-yl}methyl)pyrazolo[3,4-b]pyrazin-6-amine (27 mg, 17.3%) as a white solid. ¹H NMR (400 MHZ, DMSO-d₆) δ 8.55-8.39 (m, 1H), 8.27 (s, 1H), 8.11 (s, 1H), 8.02-7.94 (m, 1H), 7.20-7.05 (m, 1H), 6.57-6.20 (m, 1H), 4.75-4.57 (m, 2H), 3.74-3.53 (m, 2H), 3.47-3.36 (m, 2H), 3.23-3.14 (m, 3H), 2.93-2.83 (m, 1H), 2.82-2.69 (m, 1H), 2.26-2.14 (m, 1H), 2.07 (s, 1H), 1.85-1.73 (m, 2H), 1.63-1.48 (m, 1H), 1.36-1.17 (m, 1H). MS m/z: 405.15 [M+H]⁺.

### 2-((1R,5S,6r)-3-(1-(Oxetan-3-yl)-1H-pyrazolo[3,4-b]pyrazin-6-yl)-3-azabicyclo[3.1.0]hexan-6-yl)-5-(trifluoromethyl)benzo[d]thiazole (290)

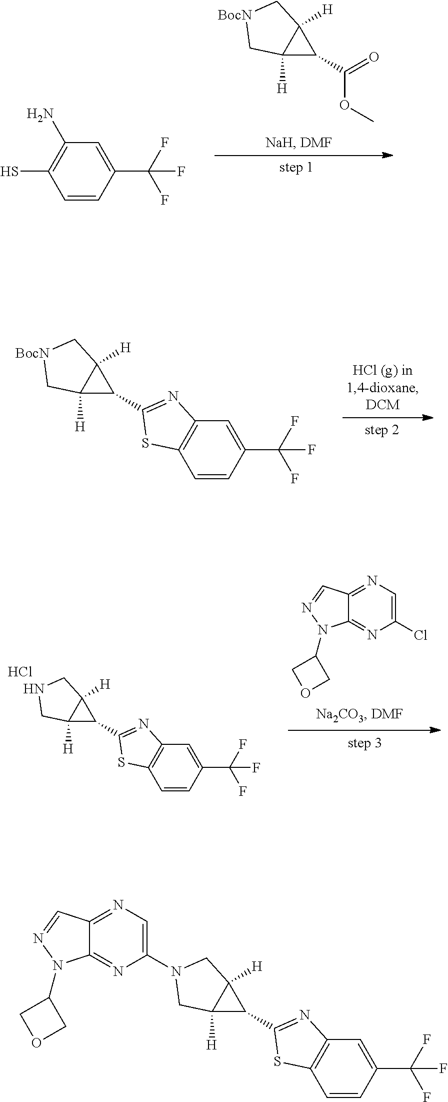

Step 1: tert-butyl (1R,5S,6r)-6-(5-(trifluoromethyl)benzo[d]thiazol-2-yl)-3-azabicyclo[3.1.0]hexane-3-carboxylate: To a stirred mixture of (1R,5S,6R)-3-(tert-butoxycarbonyl)-3-azabicyclo[3.1.0]hexane-6-carboxylic acid (300 mg, 1.32 mmol, 1 equiv) and NaH (63.4 mg, 2.640 mmol, 2 equiv) in DMF (5 mL) was added 2-amino-4-(trifluoromethyl)benzenethiol (306 mg, 1.58 mmol, 1.2 equiv) at 0° C. The resulting mixture was stirred for 3 h at room temperature. The reaction was monitored by LCMS. The mixture was quenched by water (10 mL), and extracted with EtOAc (3×10 mL). The organic layers were combined, washed with brine, dried, evaporated, and purified with a silico gel column, eluted with gradient of Hexane/EtOAc (1/1). The fractions were collected, and concentrated to give tert-butyl 1-oxo-2-(6-(trifluoromethyl)pyridin-2-yl)-2,8-diazaspiro[4.5]decane-8-carboxylate (220 mg, 46%) as a colorless oil. MS m/z: 385 [M+H]⁺.

Step 2: 2-((1R,5S,6r)-3-azabicyclo[3.1.0]hexan-6-yl)-5-(trifluoromethyl)benzo[d]thiazole hydrochloride: Followed General Procedure B using tert-butyl (1R,5S,6R)-6-[5-(trifluoromethyl)-1,3-benzothiazol-2-yl]-3-azabicyclo[3.1.0]hexane-3-carboxylate (120 mg, 0.312 mmol, 1 equiv) to afford the crude product 2-((1R,5S,6r)-3-azabicyclo[3.1.0]hexan-6-yl)-5-(trifluoromethyl)benzo[d]thiazole hydrochloride (100 mg). MS m/z: 285 [M+H]⁺.

Step 3: 2-((1R,5S,6r)-3-(1-(oxetan-3-yl)-1H-pyrazolo[3,4-b]pyrazin-6-yl)-3-azabicyclo[3.1.0]hexan-6-yl)-5-(trifluoromethyl)benzo[d]thiazole: Followed General Procedure C using 2-[(1R,5S,6S)-3-azabicyclo[3.1.0]hexan-6-yl]-5-(trifluoromethyl)-1,3-benzothiazole hydrochloride (100 mg, 0.352 mmol, 1 equiv) and 6-chloro-1-(oxetan-3-yl)pyrazolo[3,4-b]pyrazine (81.5 mg, 0.387 mmol, 1.1 equiv). The crude product was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 2-[(1R,5S,6S)-3-[1-(oxetan-3-yl)pyrazolo[3,4-b]pyrazin-6-yl]-3-azabicyclo[3.1.0]hexan-6-yl]-5-(trifluoromethyl)-1,3-benzothiazole (8 mg, 4.89%) as an white solid. ¹H NMR (400 MHZ, DMSO-d₆) δ 8.30 (d, J=8.3 Hz, 1H), 8.22 (d, J=5.0 Hz, 2H), 8.13 (s, 1H), 7.71 (d, J=8.5 Hz, 1H), 5.90 (p, J=7.2 Hz, 1H), 5.07 (t, J=6.4 Hz, 2H), 4.98 (t, J=7.1 Hz, 2H), 4.14 (d, J=11.4 Hz, 2H), 3.76-3.70 (m, 2H), 2.60 (s, 3H). MS m/z: 460.2 [M+H]⁺.

### 1-(2,2-Difluoroethyl)-6-(3-methoxy-5-(((2-(trifluoromethyl)pyridin-3-yl)oxy)methyl)piperidin-1-yl)-1H-pyrazolo[3,4-b]pyrazine (291)

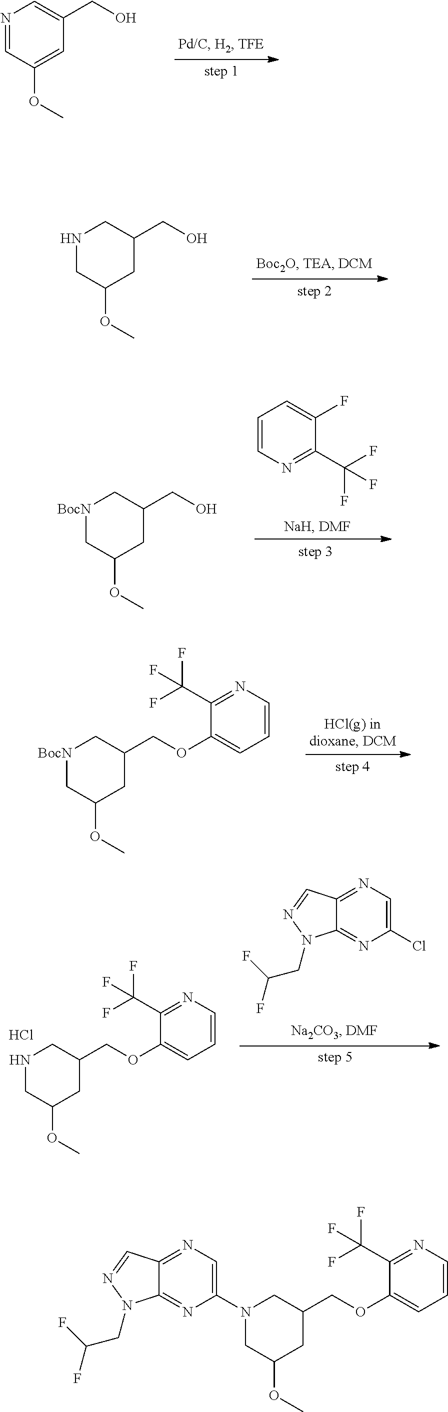

Step 1: (5-methoxypiperidin-3-yl)methanol: A mixture of (5-methoxypyridin-3-yl)methanol (500 mg, 3.59 mmol, 1 equiv) and Pd/C (150 mg) in TFE (5 mL) was stirred for overnight at 50° C. under hydrogen 60 atm. The reaction was monitored by LCMS. Desired product could be detected by LCMS. The resulting mixture was filtered, and the filter cake was washed with DCM (3×10 mL). The filtrate was concentrated under reduced pressure. The crude resulting mixture was used in the next step directly without further purification. This provided (5-methoxypiperidin-3-yl)methanol (400 mg, 65.9%) as a colorless oil. MS m/z: 146 [M+H]⁺.

Step 2: tert-butyl 3-(hydroxymethyl)-5-methoxypiperidine-1-carboxylate: To a stirred mixture of (5-methoxypiperidin-3-yl)methanol (200 mg, 1.37 mmol, 1 equiv) and Boc₂O (390 mg, 1.79 mmol, 1.3 equiv) in DCM (2 mL) was added TEA (418 mg, 4.13 mmol, 3 equiv) in portions at 0° C. under nitrogen atmosphere. The resulting mixture was stirred for 2 h at room temperature under nitrogen atmosphere. The reaction was monitored by LCMS. Desired product could be detected by LCMS. The resulting mixture was concentrated under reduced pressure. The residue was purified by silica gel column chromatography, eluted with PE/EA (5:1) to afford tert-butyl 3-(hydroxymethyl)-5-methoxypiperidine-1-carboxylate (230 mg, 64.6%) as a white oil. MS m/z: 246 [M+H]⁺.

Step 3: tert-butyl 3-methoxy-5-(((2-(trifluoromethyl)pyridin-3-yl)oxy)methyl)piperidine-1-carboxylate: Followed General Procedure D using 3-fluoro-2-(trifluoromethyl)pyridine (174 mg, 1.05 mmol, 1.3 equiv) and tert-butyl 3-(hydroxymethyl)-5-methoxypiperidine-1-carboxylate (200 mg, 0.815 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 3-methoxy-5-(((2-(trifluoromethyl)pyridin-3-yl)oxy)methyl)piperidine-1-carboxylate (220 mg, 67.0%) as a colorless oil. MS m/z: 391 [M+H]⁺.

Step 4: 3-((5-methoxypiperidin-3-yl)methoxy)-2-(trifluoromethyl)pyridine hydrochloride: Followed General Procedure B using 3-methoxy-5-({[2-(trifluoromethyl)pyridin-3-yl]oxy}methyl)piperidine-1-carboxylate (150 mg, 0.384 mmol, 1 equiv) to afford the crude product 3-((5-methoxypiperidin-3-yl)methoxy)-2-(trifluoromethyl)pyridine hydrochloride (100 mg, 85.1%). MS m/z: 291 [M+H]⁺.

Step 5: 1-(2,2-difluoroethyl)-6-(3-methoxy-5-(((2-(trifluoromethyl)pyridin-3-yl)oxy)methyl)piperidin-1-yl)-1H-pyrazolo[3,4-b]pyrazine: Followed General Procedure C using 3-[(5-methoxypiperidin-3-yl)methoxy]-2-(trifluoromethyl)pyridine hydrochloride (40 mg, 0.138 mmol, 1.0 equiv) and 6-chloro-1-(2,2-difluoroethyl)-1H-pyrazolo[3,4-b]pyrazine (30.1 mg, 0.138 mmol, 1.0 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(3-methoxy-5-(((2-(trifluoromethyl)pyridin-3-yl)oxy)methyl)piperidin-1-yl)-1H-pyrazolo[3,4-b]pyrazine (20 mg, 30.7%) as a white solid. ¹H NMR (400 MHZ, DMSO-d₆) δ 8.48 (s, 1H), 8.27 (dd, J=4.6, 1.2 Hz, 1H), 8.14 (s, 1H), 7.91-7.71 (m, 1H), 7.75-7.63 (m, 1H), 6.40 (t, J=54.9, 3.9 Hz, 1H), 4.87-4.45 (m, 4H), 4.34-4.14 (m, 1H), 4.14-3.92 (m, 1H), 3.37 (s, 4H), 3.06-2.93 (m, 2H), 2.22-2.17 (m, 1H), 1.43-1.33 (m, 1H). MS m/z: 473.1 [M+H]⁺

### (4-Fluoro-7-phenylpyrazolo[1,5-a]pyridin-3-yl)(3-((o-tolyloxy)methyl)piperidin-1-yl)methanone (292)

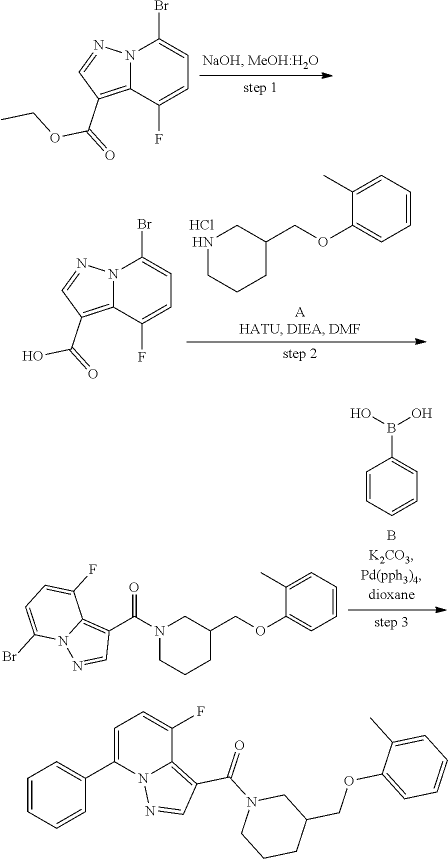

Step 1: 7-bromo-4-fluoropyrazolo[1,5-a]pyridine-3-carboxylic acid: To a stirred solution of ethyl 7-bromo-4-fluoropyrazolo[1,5-a]pyridine-3-carboxylate (170 mg, 0.592 mmol, 1 equiv) and H₂O (2 mL) in MeOH (2 mL) was added NaOH (94.7 mg, 2.36 mmol, 4 equiv) at 0° C. The mixture was allowed to reacted at room temperature for 2 h. The mixture was acidified to pH 2 with HCl (aq.). The resulting mixture was extracted with EtOAc (3×10 mL). The combined organic layers were washed with brine (10 mL), dried over anhydrous Na₂SO₄. After filtration, the filtrate was concentrated under reduced pressure. The crude product 7-bromo-4-fluoropyrazolo[1,5-a]pyridine-3-carboxylic acid (100 mg, 65.2%) was used in the next step directly without further purification. MS m/z: 259 [M+H]⁺.

Step 2: (7-bromo-4-fluoropyrazolo[1,5-a]pyridin-3-yl)(3-((o-tolyloxy)methyl)piperidin-1-yl)methanone: A solution of 7-bromo-4-fluoropyrazolo[1,5-a]pyridine-3-carboxylic acid (100 mg, 0.386 mmol, 1 equiv) and 3-(2-methylphenoxymethyl)piperidine hydrochloride (93.33 mg, 0.386 mmol, 1 equiv) and HATU (146.79 mg, 0.386 mmol, 1 equiv) and DIEA (149.69 mg, 1.158 mmol, 3 equiv) in DMF (2 mL) was stirred for 2 h at room temperature. The resulting mixture was diluted with EtOAc (20 mL). The combined organic layers were washed with brine (20 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 10% to 100% gradient in 20 min; detector, UV 254 nm. This provided (7-bromo-4-fluoropyrazolo[1,5-a]pyridin-3-yl)(3-((o-tolyloxy)methyl)piperidin-1-yl)methanone (40 mg, 23.2%) as a yellow oil. MS m/z: 446 [M+H]⁺.

Step 3: (4-fluoro-7-phenylpyrazolo[1,5-a]pyridin-3-yl)(3-((o-tolyloxy)methyl)piperidin-1-yl)methanone: A solution of (7-bromo-4-fluoropyrazolo[1,5-a]pyridin-3-yl)(3-((o-tolyloxy)methyl)piperidin-1-yl)methanone (10 mg, 0.022 mmol, 1 equiv) and phenyl boronic acid (5.46 mg, 0.044 mmol, 2 equiv) and K₂CO₃ (9.29 mg, 0.066 mmol, 3 equiv) and Pd(PPh₃)+(2.59 mg, 0.002 mmol, 0.1 equiv) in dioxane (1 mL) was stirred for overnight at 100° C. under nitrogen atmosphere. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This provided (4-fluoro-7-phenylpyrazolo[1,5-a]pyridin-3-yl)(3-((o-tolyloxy)methyl)piperidin-1-yl)methanone (4.2 mg, 41.0%) as a white solid. ¹H NMR (400 MHZ, Methanol-d₄) δ 8.04 (s, 1H), 7.91-7.79 (m, 2H), 7.62-7.50 (m, 3H), 7.35-6.92 (m, 3H), 6.90-6.68 (m, 3H), 4.22-3.81 (m, 3H), 3.78-3.65 (m, 1H), 3.58-3.41 (m, 2H), 2.25 (s, 1H), 2.13 (s, 1H), 1.98 (s, 1H), 1.87 (s, 1H), 1.70 (s, 1H), 1.64-1.55 (m, 1H), 1.51 (s, 2H). MS m/z: 444.1 [M+H]⁺.

### 6-((1R,5S,6R)-6-((3,5-Difluorophenoxy)methyl)-3-azabicyclo[3.1.0]hexan-3-yl)-1-methyl-2-(6-(trifluoromethyl)pyridin-2-yl)-1H-imidazo[4,5-b]pyrazine (293)

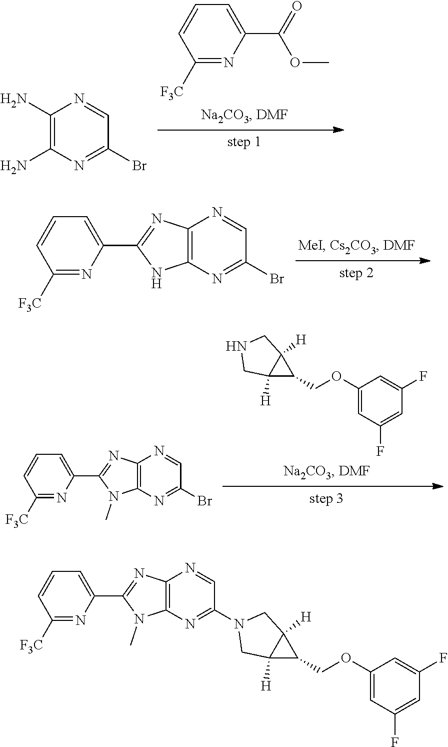 [SMILES: COC(=O)c1cccc(C(F)(F)F)n1 ; Nc1ncc(Br)nc1N ; FC(F)(F)c1cccc(-c2nc3ncc(Br)nc3[nH]2)n1 ; Cn1c(-c2cccc(C(F)(F)F)n2)nc2ncc(Br)nc21 ; Fc1cc(F)cc(OC[C@H]2[C@@H]3CNC[C@@H]32)c1 ; Cn1c(-c2cccc(C(F)(F)F)n2)nc2ncc(N3C[C@@H]4[C@@H](COc5cc(F)cc(F)c5)[C@@H]4C3)nc21]

Step 1: 6-bromo-2-(6-(trifluoromethyl)pyridin-2-yl)-1H-imidazo[4,5-b]pyrazine: To a stirred mixture of 5-bromopyrazine-2,3-diamine (101 mg, 0.536 mmol, 1.1 equiv) and NaH (23.4 mg, 0.974 mmol, 2 equiv) in DMF (2 mL) was added methyl 6-(trifluoromethyl)pyridine-2-carboxylate (100 mg, 0.487 mmol, 1 equiv). The resulting mixture was stirred for 3 h at 50° C. The reaction was monitored by LCMS. The mixture was diluted with water (10 mL), and extracted with EtOAc (3×10 mL). The organic layer was combined, washed with brine, dried, evaporated, and purified with a silico gel column, eluted with gradient of Hexane/EtOAc. The fractions were collected, and concentrated to give 6-bromo-2-(6-(trifluoromethyl)pyridin-2-yl)-1H-imidazo[4,5-b]pyrazine (80 mg, 47.7%) as a colorless oil. MS m/z: 344 [M+H]⁺.

Step 2: 6-bromo-1-methyl-2-(6-(trifluoromethyl)pyridin-2-yl)-1H-imidazo[4,5-b]pyrazine: A solution of 6-bromo-2-(6-(trifluoromethyl)pyridin-2-yl)-1H-imidazo[4,5-b]pyrazine (80 mg, 0.232 mmol, 1 equiv) and MeI (39.6 mg, 0.278 mmol, 1.2 equiv) and Cs₂CO₃ (151 mg, 0.464 mmol, 2 equiv) in DMF (2 mL) was stirred for 2 hours at 0° C. The residue was purified by reverse flash chromatography with the following conditions: column, silica gel; mobile phase, MeCN in water, 10% to 50% gradient in 10 min; detector, UV 254 nm. This provided 6-bromo-1-methyl-2-(6-(trifluoromethyl)pyridin-2-yl)-1H-imidazo[4,5-b]pyrazine (60 mg, 72.0%) as a white solid. MS m/z: 358 [M+H]⁺.

Step 3: 6-((1R,5S,6R)-6-((3,5-difluorophenoxy)methyl)-3-azabicyclo[3.1.0]hexan-3-yl)-1-methyl-2-(6-(trifluoromethyl)pyridin-2-yl)-1H-imidazo[4,5-b]pyrazine: To a stirred solution of (1R,5S,6R)-6-(3,5-difluorophenoxymethyl)-3-azabicyclo[3.1.0]hexane (41.5 mg, 0.185 mmol, 1.1 equiv) and 6-bromo-1-methyl-2-(6-(trifluoromethyl)pyridin-2-yl)-1H-imidazo[4,5-b]pyrazine (60 mg, 0.168 mmol, 1.00 equiv) in DMF (2.00 mL) was added Na₂CO₃ (35.5 mg, 0.336 mmol, 2 equiv) at room temperature. The resulting mixture was stirred for 2 h at 100° C. under nitrogen atmosphere. The resulting mixture was diluted with water (20 mL) and extracted with EtOAc (3×15 mL). The combined organic layers were washed with brine (30 mL), dried over anhydrous Na₂SO₄. After filtration, the filtrate was concentrated to dryness under reduced pressure. The residue was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 6-((1R,5S,6R)-6-((3,5-difluorophenoxy)methyl)-3-azabicyclo[3.1.0]hexan-3-yl)-1-methyl-2-(6-(trifluoromethyl)pyridin-2-yl)-1H-imidazo[4,5-b]pyrazine (12 mg, 13.4%) as an white solid. ¹H NMR (400 MHZ, DMSO-d₆) δ 8.57 (d, J=8.2 Hz, 1H), 8.27 (t, J=7.9 Hz, 1H), 8.00 (t, J=3.9 Hz, 2H), 6.81-6.70 (m, 3H), 4.14 (s, 3H), 3.98 (d, J=7.1 Hz, 2H), 3.90 (d, J=10.7 Hz, 2H), 3.58 (d, J=10.3 Hz, 2H), 1.87 (s, 2H), 1.17 (t, J=3.4 Hz, 1H). MS m/z: 502.9 [M+H]⁺.

### (1-Methyl-2,7-diphenyl-1H-pyrrolo[3,2-b]pyridin-3-yl)(3-((o-tolyloxy)methyl)piperidin-1-yl)methanone (294)

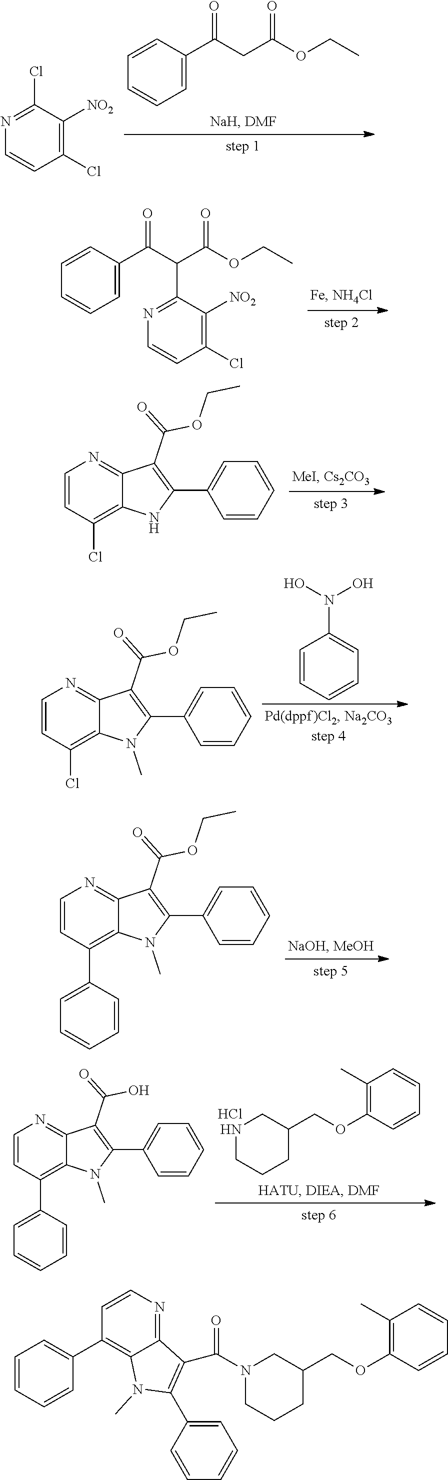

Step 1: ethyl 2-(4-chloro-3-nitropyridin-2-yl)-3-oxo-3-phenylpropanoate: To a stirred mixture of (2-chloro-3-nitropyridin-4-yl)chloranium (200 mg, 1.03 mmol, 1 equiv) and NaH (74.2 mg, 3.09 mmol, 3 equiv) in DMF (4 mL) was added ethyl benzoylacetate (238 mg. 1.24 mmol, 1.2 equiv) at room temperature under air atmosphere. The resulting mixture was stirred for 3 h at room temperature. Desired product could be detected by LCMS. The reaction was quenched with water at 0° C. The resulting mixture was extracted with EtOAc (3×30 mL). The combined organic layers were washed with water (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This provided ethyl 2-(4-chloro-3-nitropyridin-2-yl)-3-oxo-3-phenylpropanoate (80 mg, 44.2%) as a white solid. MS m/z: 349 [M+H]⁺.

Step 2: ethyl 7-chloro-2-phenyl-1H-pyrrolo[3,2-b]pyridine-3-carboxylate: To a stirred solution of ethyl 2-(4-chloro-3-nitropyridin-2-yl)-3-oxo-3-phenylpropanoate (100 mg, 0.287 mmol, 1 equiv) and Fe (96.1 mg, 1.72 mmol, 6 equiv) in EtOH (1 mL)/H₂O (1 mL) was added NH₄Cl (92.0 mg, 1.72 mmol, 6 equiv). The resulting mixture was stirred for 2 h at 80° C. Desired product could be detected by LCMS. The resulting mixture was filtered. The filter cake was washed with MeOH (3×10 mL). The filtrate was concentrated under reduced pressure. The residue was purified by reverse flash chromatography with the following conditions: column, silica gel; mobile phase, MeCN in water, 0% to 100% gradient in 30 min; detector, UV 254 nm. This provided ethyl 7-chloro-2-phenyl-1H-pyrrolo[3,2-b]pyridine-3-carboxylate (50 mg, 57.9%) as a light yellow solid. MS m/z: 301 [M+H]⁺.

Step 3: ethyl 7-chloro-1-methyl-2-phenyl-1H-pyrrolo[3,2-b]pyridine-3-carboxylate: To a stirred solution of ethyl 7-chloro-2-phenyl-1H-pyrrolo[3,2-b]pyridine-3-carboxylate (50 mg, 0.166 mmol, 1 equiv) and MeI (35.4 mg, 0.249 mmol, 1.5 equiv) in DMF (1 mL) was added Cs₂CO₃ (162 mg, 0.498 mmol, 3 equiv). The resulting mixture was stirred for 2 h at room temperature. The residue was purified by reverse flash chromatography with the following conditions: column, silica gel; mobile phase, MeCN in water, 0% to 100% gradient in 30 min; detector, UV 254 nm. This provided ethyl 7-chloro-1-methyl-2-phenylpyrrolo[3,2-b]pyridine-3-carboxylate (50 mg, 95.5%) as a light yellow solid. MS m/z: 315[M+H]⁺.

Step 4: ethyl 1-methyl-2,7-diphenyl-1H-pyrrolo[3,2-b]pyridine-3-carboxylate: To a stirred solution of ethyl 7-chloro-1-methyl-2-phenyl-1H-pyrrolo[3,2-b]pyridine-3-carboxylate (45 mg, 0.143 mmol, 1 equiv) and phenylboronic acid (26.2 mg, 0.214 mmol, 1.5 equiv) in dioxane (0.5 mL)/H₂O (0.1 mL) were added Pd(dppf)Cl₂ (8.43 mg, 0.014 mmol, 0.1 equiv) and Na₂CO₃ (45.5 mg, 0.429 mmol, 3 equiv). The resulting mixture was stirred for 3 h at 100° C. under nitrogen atmosphere. The resulting mixture was concentrated under vacuum. The residue was purified by silica gel column chromatography, eluted with PE/EtOAc (1:1) to afford ethyl 1-methyl-2,7-diphenyl-1H-pyrrolo[3,2-b]pyridine-3-carboxylate (50 mg, 98.1%) as a light yellow oil. MS m/z: 357[M+H]⁺.

Step 5: 1-methyl-2,7-diphenyl-1H-pyrrolo[3,2-b]pyridine-3-carboxylic acid: A solution of ethyl 1-methyl-2,7-diphenyl-1H-pyrrolo[3,2-b]pyridine-3-carboxylate (50 mg, 0.140 mmol, 1 equiv) and NaOH (22.4 mg, 0.560 mmol, 4 equiv) in H₂O (0.5 mL)/MeOH (0.5 mL) was stirred for 2 h at 50° C. Desired product could be detected by LCMS. The residue was purified by reverse flash chromatography with the following conditions: column, silica gel; mobile phase, MeCN in water, 0% to 100% gradient in 15 min; detector, UV 254 nm. This provided 1-methyl-2,7-diphenyl-1H-pyrrolo[3,2-b]pyridine-3-carboxylic acid (40 mg, 86.8%) as a white solid. MS m/z: 329 [M+H]⁺.

Step 6: (1-methyl-2,7-diphenyl-1H-pyrrolo[3,2-b]pyridin-3-yl)(3-((o-tolyloxy)methyl)piperidin-1-yl)methanone: To a stirred solution of 1-methyl-2,7-diphenyl-1H-pyrrolo[3,2-b]pyridine-3-carboxylic acid (25 mg, 0.076 mmol, 1.00 equiv) and HATU (31.8 mg, 0.084 mmol, 1.1 equiv) in DMF (0.5 mL) were added DIPEA (29.5 mg, 0.228 mmol, 3 equiv) and 3-((o-tolyloxy)methyl)piperidine hydrochloride (22.1 mg, 0.091 mmol, 1.2 equiv) dropwise at 0° C. The resulting mixture was stirred for 3 h at room temperature. The residue was purified by reverse flash chromatography with the following conditions: column, silica gel; mobile phase, MeCN in water, 0% to 100% gradient in 20 min; detector, UV 254 nm. This provided (1-methyl-2,7-diphenyl-1H-pyrrolo[3,2-b]pyridin-3-yl)(3-((o-tolyloxy)methyl)piperidin-1-yl)methanone (5 mg, 12.7%) as a white solid. ¹H NMR (400 MHz, DMSO-d₆) δ 8.28-8.26 (m, 1H), 7.72-7.64 (m, 2H), 7.58-7.47 (m, 9H), 7.14-7.09 (m, 2H), 6.86-6.78 (m, 2H), 4.37-4.30 (m, 1H), 3.93-3.71 (m, 2H), 3.19 (s, 3H), 2.79 (s, 2H), 2.16-2.07 (m, 1H), 1.76-1.70 (m, 4H), 1.39-1.17 (m, 3H). MS m/z: 516.4 [M+H]⁺.

### 1-(2,2-Difluoroethyl)-6-(2-(methoxymethyl)-5-(((2-(trifluoromethyl)pyridin-3-yl)oxy)methyl)piperidin-1-yl)-1H-pyrazolo[3,4-b]pyrazine (295)

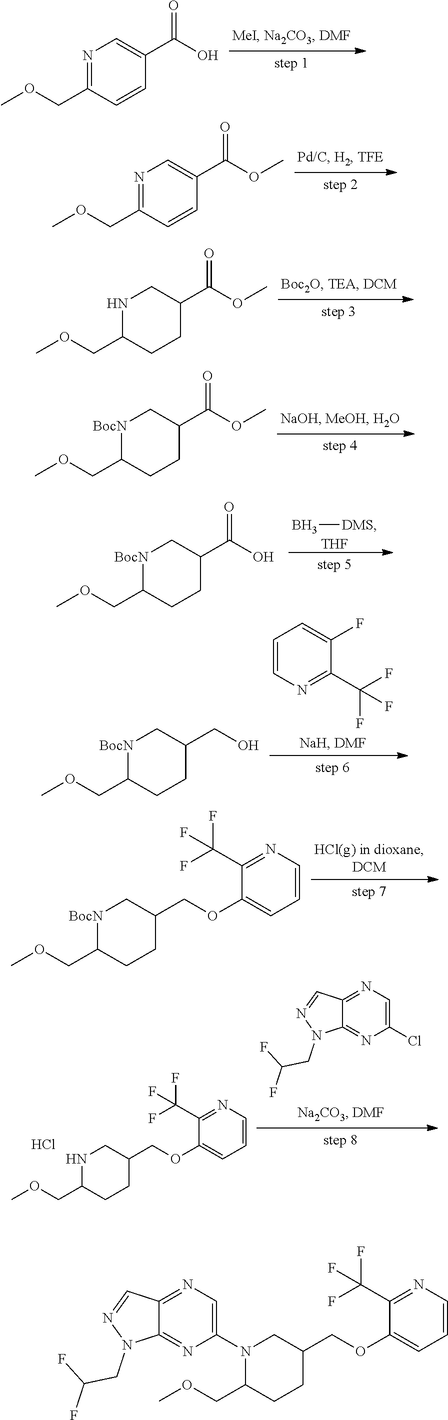

Step 1: methyl 6-(methoxymethyl)nicotinate: To a stirred mixture of 6-(methoxymethyl)nicotinic acid (500 mg, 2.99 mmol, 1 equiv) and Na₂CO₃ (951 mg, 8.97 mmol, 3 equiv) in DMF (10 mL) was added MeI (478 mg, 11.9 mmol, 4 equiv) dropwise 0° C. under nitrogen atmosphere. The resulting mixture was stirred for overnight at room temperature under nitrogen atmosphere. Desired product could be detected by LCMS. The residue was purified by reverse flash chromatography with the following conditions: column, C18 silica gel; mobile phase, MeCN in Water (0.1% FA), 0% to 100% gradient in 20 min; detector, UV 254 nm. This provided methyl methyl 6-(methoxymethyl)nicotinate (360 mg, 59.7%) as a white oil. MS m/z: 182 [M+H]⁺.

Step 2: methyl 6-(methoxymethyl)piperidine-3-carboxylate: To a stirred mixture of methyl 6-(methoxymethyl)nicotinate (430 mg, 2.37 mmol, 1 equiv) in TFE (10 mL) was added Pd/C (120 mg) with water in portions at room temperature under hydrogen atmosphere. The resulting mixture was stirred for 2 days at 60° C. under hydrogen (50 atm). The mixture was allowed to cool down to room temperature. The reaction was monitored by LCMS. Desired product could be detected by LCMS. The resulting mixture was filtered, and the filter cake was washed with DCM (3×5 mL). The filtrate was concentrated under reduced pressure. The crude resulting mixture was used in the next step directly without further purification. MS m/z: 188 [M+H]⁺.

Step 3: 1-(tert-butyl) 3-methyl 6-(methoxymethyl)piperidine-1,3-dicarboxylate: To a stirred mixture of methyl 6-(methoxymethyl)piperidine-3-carboxylate (400 mg, 2.13 mmol, 1 equiv) and Boc₂O (699.37 mg, 3.20 mmol, 1.5 equiv) in DCM (10 mL) was added TEA (432 mg, 4.27 mmol, 2 equiv) in portions at 0° C. under nitrogen atmosphere. The resulting mixture was stirred for 2 h at room temperature under nitrogen atmosphere. The reaction was monitored by LCMS. Desired product could be detected by LCMS. The residue was purified by silica gel column chromatography, eluted with PE/EA (1:1) to afford 1-tert-butyl 3-methyl 6-(methoxymethyl)piperidine-1,3-dicarboxylate (420 mg, 66.3%) as a white oil. MS m/z: 288 [M+H]⁺.

Step 4: 1-(tert-butoxycarbonyl)-6-(methoxymethyl)piperidine-3-carboxylic acid: To a stirred mixture of 1-tert-butyl 3-methyl 6-(methoxymethyl)piperidine-1,3-dicarboxylate (500 mg, 1.74 mmol, 1 equiv) in MeOH (2 mL) was added NaOH (278 mg, 6.96 mmol, 4 equiv) in portions at 0° C. under nitrogen atmosphere. The resulting mixture was stirred for 2 h at room temperature under nitrogen atmosphere. The resulting mixture was stirred for 2 h at room temperature under nitrogen atmosphere. Desired product could be detected by LCMS. The residue was acidified to pH 6 with conc. HCl. The resulting mixture was extracted by EtOAc (3×10 mL). The combined organic phases were washed by water (2×15 mL) and brine (1×15 mL), dried over anhydrous Na₂SO₄, filtrated, and concentrated. The crude product was used in the next step directly without further purification. MS m/z: 274 [M+H]⁺.

Step 5: tert-butyl 5-(hydroxymethyl)-2-(methoxymethyl)piperidine-1-carboxylate: To a stirred mixture of 1-(tert-butoxycarbonyl)-6-(methoxymethyl)piperidine-3-carboxylic acid (100 mg, 0.366 mmol, 1 equiv) in THF (1.5 mL) was added BH₃—Me₂S (83.3 mg, 1.09 mmol, 3 equiv) in portions at 0° C. under nitrogen atmosphere. The resulting mixture was stirred for 2 h at 0° C. under nitrogen atmosphere. The reaction was quenched with water at 0° C. The resulting mixture was extracted by EtOAc (3×10 mL). The combined organic phases were washed by water (2×15 mL) and brine (1×15 mL), dried over anhydrous Na₂SO₄, filtrated, and concentrated. The residue was purified by reverse flash chromatography with the following conditions: column, C18 silica gel; mobile phase, MeCN in water, 10% to 100% gradient in 10 min; detector, UV 254 nm. This provided tert-butyl 5-(hydroxymethyl)-2-(methoxymethyl)piperidine-1-carboxylate (80 mg, 75.8%) as a white oil. MS m/z: 260 [M+H]⁺.

Step 6: tert-butyl 2-(methoxymethyl)-5-(((2-(trifluoromethyl)pyridin-3-yl)oxy)methyl)piperidine-1-carboxylate: Followed General Procedure D using 3-fluoro-2-(trifluoromethyl)pyridine (66.8 mg, 0.405 mmol, 1.5 equiv) and tert-butyl 5-(hydroxymethyl)-2-(methoxymethyl)piperidine-1-carboxylate (70 mg, 0.270 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 2-(methoxymethyl)-5-(((2-(trifluoromethyl)pyridin-3-yl)oxy)methyl)piperidine-1-carboxylate (80 mg, 73.2%) as a white oil. MS m/z: 405 [M+H]⁺.

Step 7: 3-((6-(methoxymethyl)piperidin-3-yl)methoxy)-2-(trifluoromethyl)pyridine hydrochloride: Followed General Procedure B using tert-butyl 2-(methoxymethyl)-5-(((2-(trifluoromethyl)pyridin-3-yl)oxy)methyl)piperidine-1-carboxylate (80 mg, 0.198 mmol, 1 equiv) to afford the crude product 3-((6-(methoxymethyl)piperidin-3-yl)methoxy)-2-(trifluoromethyl)pyridine hydrochloride (45 mg) was used for next step without further purification. MS m/z: 305 [M+H]⁺.

Step 8: 1-(2,2-difluoroethyl)-6-(2-(methoxymethyl)-5-(((2-(trifluoromethyl)pyridin-3-yl)oxy)methyl)piperidin-1-yl)-1H-pyrazolo[3,4-b]pyrazine: Followed General Procedure C using 3-((6-(methoxymethyl)piperidin-3-yl)methoxy)-2-(trifluoromethyl)pyridine hydrochloride (20 mg, 0.0660 mmol, 1 equiv) and 6-chloro-1-(2,2-difluoroethyl)-1H-pyrazolo[3,4-b]pyrazine (15.8 mg, 0.0730 mmol, 1.1 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(2-(methoxymethyl)-5-(((2-(trifluoromethyl)pyridin-3-yl)oxy)methyl)piperidin-1-yl)-1H-pyrazolo[3,4-b]pyrazine (10 mg. 30.9%) as a white solid. ¹H NMR (400 MHZ, DMSO-d₆) δ 8.43 (s, 1H), 8.28 (d, J=4.5 Hz, 1H), 8.11 (s, 1H), 7.83 (d, J=8.5 Hz, 1H), 7.75-7.67 (m, 1H), 6.66 (s, OH), 6.63-6.18 (m, 1H), 4.89 (s, 2H), 4.73-4.60 (m, 2H), 4.30-4.23 (m, 1H), 4.14-4.05 (m, 1H), 3.75-3.66 (m, 1H), 3.54 (dd, J=10.2, 6.3 Hz, 1H), 3.25 (s, 3H), 3.18 (s, 1H), 2.96-2.85 (m, 1H), 2.08 (s, 1H), 1.88 (s, 1H), 1.78-1.67 (m, 2H), 1.56 (s, 2H), 1.37 (s, 4H), 1.23 (s, OH), 0.96 (s, OH), 0.74 (d, J=7.4 Hz, 1H). MS m/z: 487 [M+H]⁺.

### 2-(4-Fluoro-3-(3-((o-tolyloxy)methyl)piperidine-1-carbonyl)pyrazolo[1,5-a]pyridin-7-yl)benzonitrile (296)

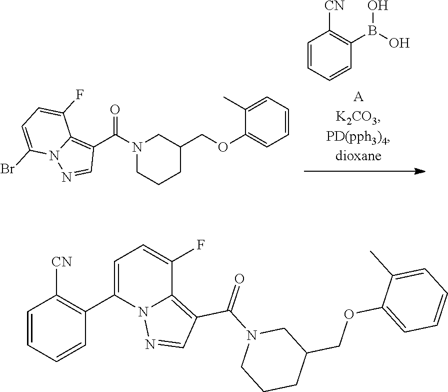 [SMILES: N#Cc1ccccc1B(O)O ; Cc1ccccc1OCC1CCCN(C(=O)c2cnn3c(Br)ccc(F)c23)C1 ; Cc1ccccc1OCC1CCCN(C(=O)c2cnn3c(-c4ccccc4C#N)ccc(F)c23)C1]

A solution of (7-bromo-4-fluoropyrazolo[1,5-a]pyridin-3-yl)(3-((o-tolyloxy)methyl)piperidin-1-yl)methanone (10 mg, 0.022 mmol, 1 equiv) and (2-cyanophenyl)boronic acid (6.58 mg, 0.044 mmol, 2 equiv) and K₂CO₃ (9.29 mg, 0.066 mmol, 3 equiv) and Pd(PPh₃)₄ (2.59 mg, 0.002 mmol, 0.1 equiv) in dioxane (1 mL) was stirred for overnight at 100° C. under nitrogen atmosphere. The resulting mixture was concentrated under vacuum. The residue was purified by reverse flash chromatography with the following conditions: column, C18 silica gel; mobile phase, MeCN in Water (0.1% FA), 10% to 100% gradient in 20 min; detector, UV 254 nm. This provided 2-(4-fluoro-3-(3-((o-tolyloxy)methyl)piperidine-1-carbonyl)pyrazolo[1,5-a]pyridin-7-yl)benzonitrile (5.5 mg, 51.0%) as a white solid. ¹H NMR (400 MHZ, DMSO-d₆) δ 8.18 (s, 1H), 8.08 (d, J=7.7 Hz, 1H), 7.93 (t, J=7.7 Hz, 1H), 7.86-7.73 (m, 2H), 7.50-7.30 (m, 1H), 7.19 (s, 1H), 7.10-7.05 (m, 1H), 7.00-6.95 (m, 1H), 6.80-6.70 (m, 2H), 4.70-4.16 (m, 1H), 4.00-3.80 (m, 2H), 3.70-3.60 (m, 1H), 3.20-2.89 (m, 2H), 2.30-2.10 (m, 1H), 2.05-1.95 (m, 1H), 1.95-1.85 (m, 1H), 1.85-1.75 (m, 1H), 1.61-1.41 (m, 4H). MS m/z: 469.1 [M+H]⁺.

### Cyclopropyl(4-(6-(3-((2-(trifluoromethyl)phenoxy)methyl)piperidin-1-yl)pyrazin-2-yl)piperidin-1-yl)methanone (297)

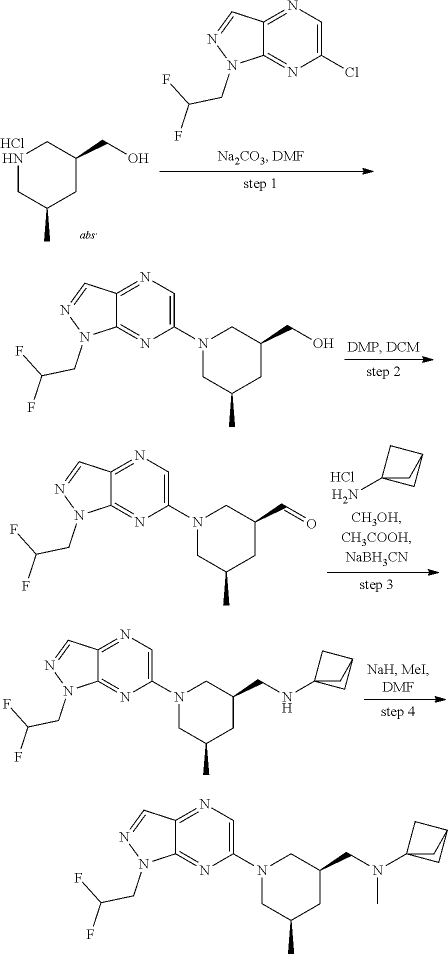

Step 1. ((3S,5R)-1-(1-(2,2-difluoroethyl)-1H-pyrazolo[3,4-b]pyrazin-6-yl)-5-methylpiperidin-3-yl)methanol: A solution of ((3S,5R)-5-methylpiperidin-3-yl)methanol hydrochloride (300 mg, 1.81 mmol, 1 equiv), 6-chloro-1-(2,2-difluoroethyl)-1H-pyrazolo[3,4-b]pyrazine (435 mg, 1.99 mmol, 1.1 equiv) and Na₂CO₃ (384 mg, 3.62 mmol, 2 equiv) in DMF (5 mL) was stirred for 2 h at 100° C. The resulting mixture was extracted with EtOAc (40 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:1) to afford ((3S,5R)-1-(1-(2,2-difluoroethyl)-1H-pyrazolo[3,4-b]pyrazin-6-yl)-5-methylpiperidin-3-yl)methanol (300 mg, 53.2%) as a white solid. MS m/z: 312 [M+H]⁺.

Step 2. (3S,5R)-1-(1-(2,2-difluoroethyl)-1H-pyrazolo[3,4-b]pyrazin-6-yl)-5-methylpiperidine-3-carbaldehyde: A solution of ((3S,5R)-1-(1-(2,2-difluoroethyl)-1H-pyrazolo[3,4-b]pyrazin-6-yl)-5-methylpiperidin-3-yl)methanol (300 mg, 0.964 mmol, 1 equiv) and DMP (613 mg, 1.45 mmol, 1.5 equiv) in DCM (5 mL) was stirred for 2 h at 0° C. The resulting mixture was concentrated under reduced pressure. The residue was purified by silica gel column chromatography, eluted with PE/EA (2:1) to afford (3S,5R)-1-(1-(2,2-difluoroethyl)-1H-pyrazolo[3,4-b]pyrazin-6-yl)-5-methylpiperidine-3-carbaldehyde (200 mg, 67.1%) as a white solid. MS m/z: 310 [M+H]⁺.

Step 3. N-(((3R,5R)-1-(1-(2,2-difluoroethyl)-1H-pyrazolo[3,4-b]pyrazin-6-yl)-5-methylpiperidin-3-yl)methyl)bicyclo[1.1.1]pentan-1-amine: A solution of (3S,5R)-1-(1-(2,2-difluoroethyl)-1H-pyrazolo[3,4-b]pyrazin-6-yl)-5-methylpiperidine-3-carbaldehyde (150 mg, 0.485 mmol, 1 equiv) and bicyclo[1.1.1]pentan-1-amine hydrochloride (116 mg, 0.97 mmol, 2 equiv) in CH₃OH (3 mL)/CH₃COOH (0.6 mL) was stirred for 2 h at 0° C. followed by the addition of NaBH₃CN (91.4 mg, 1.46 mmol, 3 equiv) at 50° C. The reaction was quenched by the addition of sat. NaHCO₃(aq.) (3 mL) at 0° C. The resulting mixture was concentrated under vacuum. The residue was purified by silica gel column chromatography, eluted with PE/EA (1:1) to afford N-(((3R,5R)-1-(1-(2,2-difluoroethyl)-1H-pyrazolo[3,4-b]pyrazin-6-yl)-5-methylpiperidin-3-yl)methyl)bicyclo[1.1.1]pentan-1-amine (40 mg, 21.9%). MS m/z: 377 [M+H]⁺.

Step 4. N-(((3R,5R)-1-(1-(2,2-difluoroethyl)-1H-pyrazolo[3,4-b]pyrazin-6-yl)-5-methylpiperidin-3-yl)methyl)-N-methylbicyclo[1.1.1]pentan-1-amine: To a stirred solution of N-(((3R,5R)-1-(1-(2,2-difluoroethyl)-1H-pyrazolo[3,4-b]pyrazin-6-yl)-5-methylpiperidin-3-yl)methyl)bicyclo[1.1.1]pentan-1-amine (40 mg, 0.106 mmol, 1 equiv) and NaH (7.65 mg, 0.318 mmol, 3 equiv) in DMF (1 mL) was added CH₃I (16.6 mg, 0.117 mmol, 1.1 equiv) dropwise at 0° C. for 2 h. The residue was purified by reverse flash chromatography with the following conditions: column, C18 silica gel; mobile phase, MeCN in water, 10% to 100% gradient in 30 min; detector, UV 254 nm. This provided N-(((3R,5R)-1-(1-(2,2-difluoroethyl)-1H-pyrazolo[3,4-b]pyrazin-6-yl)-5-methylpiperidin-3-yl)methyl)-N-methylbicyclo[1.1.1]pentan-1-amine (20 mg, 47.8%) as an off-white oil. ¹H NMR (400 MHZ, CD₃OD) δ 8.35 (s, 1H), 7.97 (s, 1H), 6.26 (tt, J=55.4, 4.1 Hz, 1H), 4.81-4.73 (m, 1H), 4.72-4.62 (m, 2H), 4.61-4.54 (m, 1H), 2.61-2.45 (m, 2H), 2.42 (s, 1H), 2.41-2.35 (m, 1H), 2.29-2.20 (m, 4H), 1.95-1.66 (m, 9H), 1.00 (d, J=6.6 Hz, 3H), 0.89 (q, J=12.0 Hz, 1H). MS m/z: 391.10 [M+H]⁺.

### 1-(2,2-Difluoroethyl)-6-((3R,4R)-4-methoxy-3-(((2-(trifluoromethyl)pyridin-3-yl)oxy)methyl)piperidin-1-yl)-1H-pyrazolo[3,4-b]pyrazine (298a); 1-(2,2-Difluoroethyl)-6-((3S,4R)-4-methoxy-3-(((2-(trifluoromethyl)pyridin-3-yl)oxy)methyl)piperidin-1-yl)-1H-pyrazolo[3,4-b]pyrazine (298b)

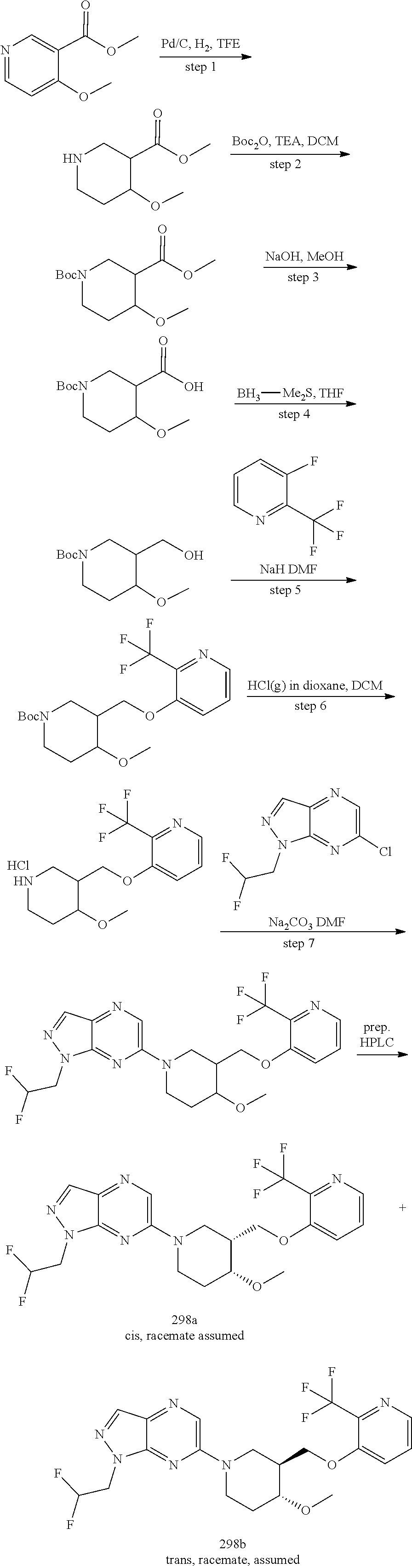

Step 1: methyl 4-methoxypiperidine-3-carboxylate: To a stirred mixture of methyl 4-methoxynicotinate (300 mg, 1.19 mmol, 1 equiv) in TFE (10 mL) was added Pd/C (60 mg) in portions at room temperature. The resulting mixture was stirred for overnight at 60° C. under H₂ (50 atm). The mixture was allowed to cool down to room temperature. The reaction was monitored by LCMS. Desired product could be detected by LCMS. The resulting mixture was filtered, and the filter cake was washed with DCM (3×5 mL). The filtrate was concentrated under reduced pressure. The residue was purified by silica gel column chromatography, eluted with PE/EA (1:1) to afford methyl 4-methoxypiperidine-3-carboxylate (200 mg, 82.0%) as a colorless oil. MS m/z: 174 [M+H]⁺.

Step 2: 1-(tert-butyl) 3-methyl 4-methoxypiperidine-1,3-dicarboxylate: To a stirred mixture of methyl 4-methoxypiperidine-3-carboxylate (200 mg, 1.15 mmol, 1 equiv) and Boc₂O (378 mg, 1.73 mmol, 1.50 equiv) in DCM (2 mL) was added TEA (175 mg, 1.73 mmol, 1.50 equiv) dropwise 0° C. under nitrogen atmosphere. The resulting mixture was stirred for 2 h at room temperature under nitrogen atmosphere. The reaction was monitored by LCMS. Desired product could be detected by LCMS. The residue was purified by silica gel column chromatography, eluted with PE/EA (1:1) to afford 1-tert-butyl 3-methyl 4-methoxypiperidine-1,3-dicarboxylate (190 mg, 60.2%) as a white oil. MS m/z: 274 [M+H]⁺.

Step 3: 1-(tert-butoxycarbonyl)-4-methoxypiperidine-3-carboxylic acid: A mixture of 1-tert-butyl 3-methyl 4-methoxypiperidine-1,3-dicarboxylate (150 mg, 0.549 mmol, 1 equiv) and NaOH (43.9 mg, 1.09 mmol, 2 equiv) in MeOH (1.5 mL) was stirred for 2 h at room temperature under nitrogen atmosphere. The reaction was monitored by LCMS. Desired product could be detected by LCMS. The mixture was acidified to pH 6 with HCl (aq.). The resulting mixture was extracted by EtOAc (3×10 mL). The combined organic phases were washed by water (2×15 mL) and brine (1×15 mL), dried over anhydrous Na₂SO₄, filtrated, and concentrated. The residue was purified by reverse flash chromatography with the following conditions: column, C18 silica gel; mobile phase, MeCN in Water (0.1% FA), 5% to 100% gradient in 20 min; detector, UV 254 nm. This provided 1-(tert-butoxycarbonyl)-4-methoxypiperidine-3-carboxylic acid (120 mg, 75.8%) as a white oil. MS m/z: 260 [M+H]⁺.

Step 4: tert-butyl 3-(hydroxymethyl)-4-methoxypiperidine-1-carboxylate: To a stirred mixture of 1-(tert-butoxycarbonyl)-4-methoxypiperidine-3-carboxylic acid (110 mg, 0.424 mmol, 1 equiv) in THF (1.5 mL) was added BH₃—Me₂S (161 mg, 2.12 mmol, 5 equiv) dropwise 0° C. under nitrogen atmosphere. The resulting mixture was stirred for 2 h at room temperature under nitrogen atmosphere. The reaction was monitored by LCMS. Desired product could be detected by LCMS. The reaction was quenched with water at 0° C. The resulting mixture was extracted by EtOAc (3×10 mL). The combined organic phases were washed by water (2×15 mL) and brine (1×15 mL), dried over anhydrous Na₂SO₄, filtrated, and concentrated. The residue was purified by silica gel column chromatography, eluted with PE/EA (1:1) to afford tert-butyl 3-(hydroxymethyl)-4-methoxypiperidine-1-carboxylate (90 mg, 86.4%) as a white oil. MS m/z: 246 [M+H]+

Step 5: tert-butyl 4-methoxy-3-(((2-(trifluoromethyl)pyridin-3-yl)oxy)methyl)piperidine-1-carboxylate: Followed General Procedure D using tert-butyl 3-(hydroxymethyl)-4-methoxypiperidine-1-carboxylate (80 mg, 0.326 mmol, 1 equiv) and 3-fluoro-2-(trifluoromethyl)pyridine (69.9 mg, 0.424 mmol, 1.3 equiv). The crude product was purified by reverse flash chromatography with the following conditions: column, C18 silica gel; mobile phase, MeCN in Water (0.1% FA), 10% to 100% gradient in 20 min; detector, UV 254 nm. This provided tert-butyl 4-methoxy-3-(((2-(trifluoromethyl)pyridin-3-yl)oxy)methyl)piperidine-1-carboxylate (45 mg, 35.3%) as a white oil. MS m/z: 391 [M+H]⁺.

Step 6: 3-((4-methoxypiperidin-3-yl)methoxy)-2-(trifluoromethyl)pyridine hydrochloride: Followed General Procedure B using tert-butyl 4-methoxy-3-({[2-(trifluoromethyl)pyridin-3-yl]oxy}methyl)piperidine-1-carboxylate (70 mg, 0.179 mmol, 1 equiv) to afford the crude product 3-((4-methoxypiperidin-3-yl)methoxy)-2-(trifluoromethyl)pyridine hydrochloride (50 mg, 86.46%). MS m/z: 291 [M+H]⁺.

Step 7: 1-(2,2-difluoroethyl)-6-(4-methoxy-3-(((2-(trifluoromethyl)pyridin-3-yl)oxy)methyl)piperidin-1-yl)-1H-pyrazolo[3,4-b]pyrazine: Followed General Procedure C using 3-((4-methoxypiperidin-3-yl)methoxy)-2-(trifluoromethyl)pyridine hydrochloride (40 mg. 0.138 mmol, 1 equiv) and 6-chloro-1-(2,2-difluoroethyl)-1H-pyrazolo[3,4-b]pyrazine (36.1 mg, 0.166 mmol, 1.2 equiv). The crude product was purified by reverse flash chromatography with the following conditions: column, C18 silica gel; mobile phase, MeCN in water, 0% to 100% gradient in 20 min; detector, UV 254 nm. This provided 1-(2,2-difluoroethyl)-6-(4-methoxy-3-(((2-(trifluoromethyl)pyridin-3-yl)oxy)methyl)piperidin-1-yl)-1H-pyrazolo[3,4-b]pyrazine (45 mg) as a white solid. This product was further purified by prep. HPLC with the following conditions: column: XBridge Prep F-Phenyl OBD Column, 19*100 mm, 5 um; Mobile Phase A: Water(0.1% FA), Mobile Phase B: ACN; Flow rate: 25 mL/min; Gradient: 45% B to 71% B in 7 min, 71% B; Wave Length: 254/220 nm; RT1(min): 6.35. This provided 1-(2,2-difluoroethyl)-6-((3R,4R)-4-methoxy-3-(((2-(trifluoromethyl)pyridin-3-yl)oxy)methyl)piperidin-1-yl)-1H-pyrazolo[3,4-b]pyrazine (298a, 15 mg, 23.0%) and 1-(2,2-difluoroethyl)-6-((3S,4R)-4-methoxy-3-(((2-(trifluoromethyl)pyridin-3-yl)oxy)methyl)piperidin-1-yl)-1H-pyrazolo[3,4-b]pyrazine (298b, 11 mg, 16.8%) as white solids.

298a: ¹H NMR (400 MHZ, DMSO-d₆) δ 8.46 (s, 1H), 8.27 (d, J=4.6 Hz, 1H), 8.14 (s, 1H), 7.83 (d, J=8.5 Hz, 1H), 7.74-7.66 (m, 1H), 6.64-6.21 (m, 1H), 4.71 (s, 1H), 4.69-4.60 (m, 2H), 4.48-4.36 (m, 2H), 4.26-4.18 (m, 1H), 3.46 (d, J=8.3 Hz, 1H), 3.23-3.07 (m, 2H), 2.30-2.21 (m, 1H), 2.06 (s, 1H), 1.42 (d, J=12.1 Hz, 1H). MS m/z: 473.1 [M+H]⁺.

298b: ¹H NMR (400 MHZ, DMSO-d₆) δ 8.43 (s, 1H), 8.28-8.22 (m, 1H), 8.12 (s. 1H), 7.82 (d, J=8.6 Hz, 1H), 7.70-7.62 (m, 1H), 6.63-6.12 (m, 1H), 4.69-4.56 (m, 2H), 4.36-4.23 (m, 2H), 4.18-4.08 (m, 1H), 4.11-4.03 (m, 1H), 3.75-3.68 (m, 1H), 3.58-3.47 (m, 1H), 3.47-3.37 (m, 1H), 2.39-2.29 (m, 1H), 2.10-1.96 (m, 1H), 1.75-1.64 (m, 1H). MS m/z: 473.1 [M+H]⁺.

### 1-(2,2-Difluoroethyl)-6-((3S,5R)-3-(difluoromethoxy)-5-(((2-(trifluoromethyl)pyridin-3-yl)oxy)methyl)piperidin-1-yl)-1H-pyrazolo[3,4-b]pyrazine (299a); 1-(2,2-Difluoroethyl)-6-((3R,5R)-3-(difluoromethoxy)-5-(((2-(trifluoromethyl)pyridin-3-yl)oxy)methyl)piperidin-1-yl)-1H-pyrazolo[3,4-b]pyrazine (299b)

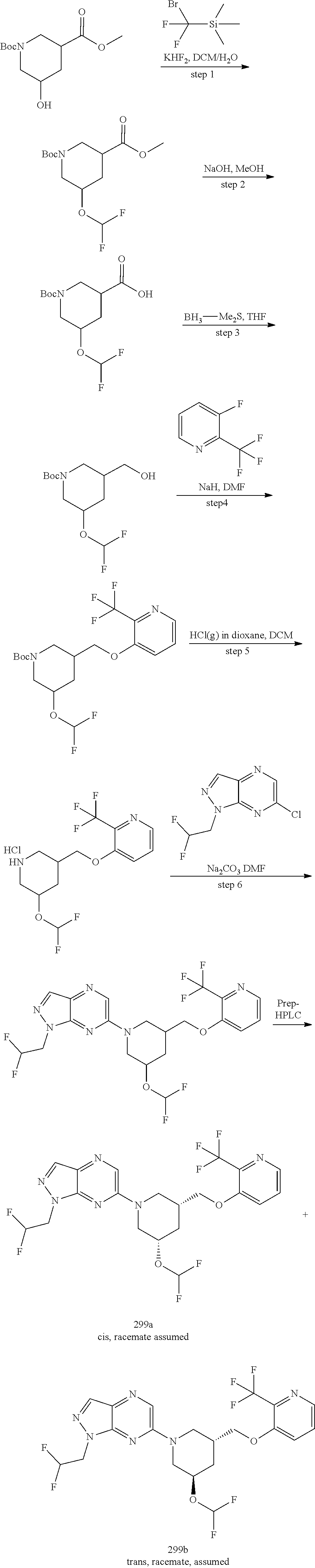

Step 1: 1-(tert-butyl) 3-methyl 5-(difluoromethoxy)piperidine-1,3-dicarboxylate: To a stirred mixture of 1-(tert-butyl) 3-methyl 5-hydroxypiperidine-1,3-dicarboxylate (25 mg, 0.096 mmol, 1.00 equiv) and (bromodifluoromethyl)trimethylsilane (39.1 mg, 0.192 mmol, 2 equiv) in DCM (0.5 mL) were added KHF₂ (15.0 mg, 0.192 mmol, 2 equiv) and H₂O (0.5 mL) in portions at 0° C. under nitrogen atmosphere. The resulting mixture was stirred for overnight at 60° C. under nitrogen atmosphere. The mixture was allowed to cool down to room temperature. The reaction was monitored by LCMS. Desired product could be detected by LCMS. The residue was purified by reverse flash chromatography with the following conditions: column, C18 silica gel; mobile phase, MeCN in water, 10% to 100% gradient in 10 min; detector, UV 254 nm. This provided 1-(tert-butyl) 3-methyl 5-(difluoromethoxy)piperidine-1,3-dicarboxylate (200 mg, 75.8%) as a white oil. MS m/z: 310 [M+H]⁺.

Step 2: 1-(tert-butoxycarbonyl)-5-(difluoromethoxy)piperidine-3-carboxylic acid: A mixture of 1-(tert-butyl) 3-methyl 5-(difluoromethoxy)piperidine-1,3-dicarboxylate (20.0 mg. 0.065 mmol, 1 equiv) and NaOH (10.3 mg, 0.260 mmol, 4 equiv) in MeOH (0.5 mL) was stirred for 2 h at room temperature under nitrogen atmosphere. The reaction was monitored by LCMS. Desired product could be detected by LCMS. The reaction was acidified to PH ˜3 by HCl (3 M) at 0° C. The resulting mixture was extracted with EtOAc (3×10 mL). The combined organic layers were washed with water (2×10 mL) and brine (1×15 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 10 min; detector, UV 254 nm. This provided 1-(tert-butoxycarbonyl)-5-(difluoromethoxy)piperidine-3-carboxylic acid (180 mg, 75.8%) as a white oil. MS m/z: 296 [M+H]⁺.

Step 3: tert-butyl 3-(difluoromethoxy)-5-(hydroxymethyl)piperidine-1-carboxylate: To a stirred mixture of 1-(tert-butoxycarbonyl)-5-(difluoromethoxy)piperidine-3-carboxylic acid (20.0 mg, 0.0608 mmol, 1 equiv) in THF (0.5 mL) was added BH₃—Me₂S (20.5 mg, 0.272 mmol, 4 equiv) in portions at 0° C. under nitrogen atmosphere. The resulting mixture was stirred for 2 h at 0° C. under nitrogen atmosphere. The reaction was monitored by LCMS. Desired product could be detected by LCMS. The reaction was quenched with water at 0° C. The resulting mixture was extracted with EtOAc (3×10 mL). The combined organic layers were washed with water (2×10 mL) and brine (1×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 10 min; detector, UV 254 nm. This provided tert-butyl 3-(difluoromethoxy)-5-(hydroxymethyl)piperidine-1-carboxylate (110 mg, 75.8%) as a white oil. MS m/z: 282 [M+H]⁺.

Step 4: tert-butyl 3-(difluoromethoxy)-5-(((2-(trifluoromethyl)pyridin-3-yl)oxy)methyl)piperidine-1-carboxylate: Followed General Procedure D using tert-butyl 3-(difluoromethoxy)-5-(hydroxymethyl)piperidine-1-carboxylate (100 mg, 0.355 mmol, 1 equiv) and 3-fluoro-2-(trifluoromethyl)pyridine (76.3 mg, 0.461 mmol, 1.3 equiv). The crude product was purified by reverse flash chromatography with the following conditions: column, C18 silica gel; mobile phase, MeCN in Water (0.1% FA), 10% to 100% gradient in 20 min; detector, UV 254 nm. This provided tert-butyl 3-(difluoromethoxy)-5-(((2-(trifluoromethyl)pyridin-3-yl)oxy)methyl)piperidine-1-carboxylate (110 mg, 72.5%) as a white oil. MS m/z: 427 [M+H]⁺.

Step 5: 3-((5-(difluoromethoxy)piperidin-3-yl)methoxy)-2-(trifluoromethyl)pyridine hydrochloride: Followed General Procedure B using tert-butyl 3-(difluoromethoxy)-5-({[2-(trifluoromethyl)pyridin-3-yl]oxy}me-thyl)piperidine-1-carboxylate (100 mg, 0.235 mmol, 1 equiv) to afford the crude product 3-((5-(difluoromethoxy)piperidin-3-yl)methoxy)-2-(trifluoromethyl)pyridine hydrochloride (90 mg) was used for next step without further purification. MS m/z: 327 [M+H]⁺.

Step 6: 1-(2,2-difluoroethyl)-6-(3-(difluoromethoxy)-5-(((2-(trifluoromethyl)pyridin-3-yl)oxy)methyl)piperidin-1-yl)-1H-pyrazolo[3,4-b]pyrazine: Followed General Procedure C using 3-{[5-(difluoromethoxy)piperidin-3-yl]methoxy}-2-(trifluoromethyl)pyridine hydrochloride (90 mg, 0.306 mmol, 1 equiv) and 6-chloro-1-(2,2-difluoroethyl)pyrazolo[3,4-b]pyrazine (73.7 mg, 0.337 mmol, 1.1 equiv. The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(3-(difluoromethoxy)-5-(((2-(trifluoromethyl)pyridin-3-yl)oxy)methyl)piperidin-1-yl)-1H-pyrazolo[3,4-b]pyrazine (40 mg) as a white solid. This product was further purified by prep. HPLC with the following conditions: column: XBridge Prep F-Phenyl OBD Column, 19*100 mm, 5 um; Mobile Phase A: Water (0.1% FA), Mobile Phase B: ACN; Flow rate: 25 mL/min; Gradient: 45% B to 71% B in 7 min, 71% B; Wave Length: 254/220 nm; RT1(min): 6.35. This provided 1-(2,2-difluoroethyl)-6-(3S,5R)-3-(difluoromethoxy)-5-(((2-(trifluoromethyl)pyridin-3-yl)oxy)methyl)piperidin-1-yl)-1H-pyrazolo[3,4-b]pyrazine (299a, 12.8 mg, 8.2%) and 1-(2,2-difluoroethyl)-6-(3S,5S)-3-(difluoromethoxy)-5-(((2-(trifluoromethyl)pyridin-3-yl)oxy)methyl)piperidin-1-yl)-1H-pyrazolo[3,4-b]pyrazine (299b, 10.4 mg, 6.6%) as white solids.

299a: ¹H NMR (400 MHZ, DMSO-d₆) δ 8.42 (s, 1H), 8.30-8.25 (m, 1H), 8.13 (s. 1H), 7.85-7.78 (m, 1H), 7.74-7.66 (m, 1H), 6.97-6.72 (m, 1H), 6.59-6.23 (m, 1H), 4.80 (d, J=13.3 Hz, 1H), 4.73-4.55 (m, 4H), 4.28-4.20 (m, 1H), 4.15-4.06 (m, 1H), 3.43 (d, J=13.2 Hz, 1H), 3.07-2.96 (m, 1H), 2.44-2.31 (m, 1H), 2.05 (s, 1H), 1.84-1.73 (m, 1H). MS m/z: 509.1 [M+H]⁺.

299b: ¹H NMR (400 MHZ, DMSO-d₆) δ 8.48 (s, 1H), 8.28 (d, J=4.5 Hz, 1H), 8.17 (s, 1H), 7.83-7.78 (m, 1H), 7.73-7.67 (m, 1H), 6.84 (s, 1H), 6.65-6.25 (m, 1H), 4.74-4.62 (m, 4H), 4.35-4.23 (m, 2H), 4.17-4.10 (m, 1H), 3.15-3.07 (m, 1H), 3.06-2.96 (m, 1H), 2.28-2.19 (m, 2H), 1.66-1.55 (m, 1H). MS m/z: 509.1 [M+H]⁺.

### (S)-4-Phenyl-6-(3-(((2-(trifluoromethyl)pyridin-3-yl)oxy)methyl)piperidin-1-yl)pyridin-2(1H)-one (300)

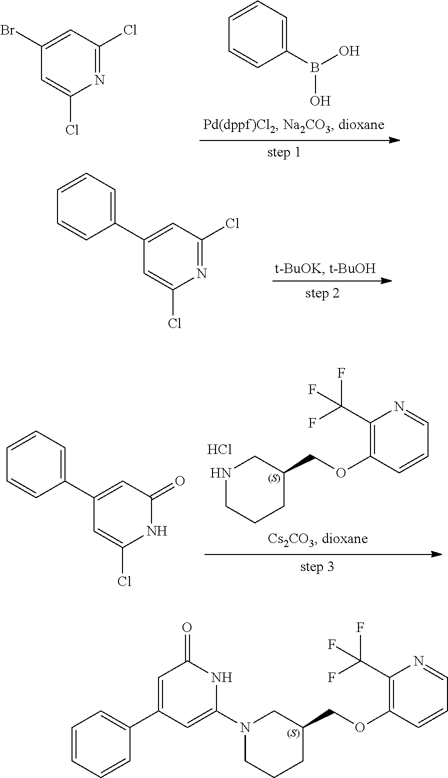

Step 1: 2,6-dichloro-4-phenylpyridine: To a stirred solution of 4-bromo-2,6-dichloropyridine (500 mg, 2.20 mmol, 1 equiv) and phenylboronic acid (322 mg, 2.65 mmol, 1.2 equiv) in dioxane (5 mL)/H₂O (1 mL) were added Pd(dppf)Cl₂ (161 mg, 0.220 mmol, 0.1 equiv) and Na₂CO₃ (700 mg, 6.61 mmol, 3 equiv). The resulting mixture was stirred for overnight at 100° C. under nitrogen atmosphere. Desired product could be detected by LCMS. The resulting mixture was concentrated under vacuum. The residue was purified by silica gel column chromatography, eluted with PE/EtOAc (1:1) to afford 2,6-dichloro-4-phenylpyridine (400 mg, 81.0%) as a light-yellow solid. MS m/z: 224 [M+H]⁺.

Step 2: 6-chloro-4-phenylpyridin-2(1H)-one: A solution of 2,6-dichloro-4-phenylpyridine (200 mg, 0.893 mmol, 1 equiv) and t-BuOK (300 mg, 2.68 mmol, 3 equiv) in t-BuOH (2 mL) was stirred for overnight at 100° C. The desired product could be detected by LCMS. The residue was purified by reverse flash chromatography with the following conditions: column, C18 silica gel; mobile phase, MeCN in Water (0.1% NH₃·H₂O), 0% to 100% gradient in 30 min; detector, UV 254 nm. This provided 6-chloro-4-phenylpyridin-2(1H)-one (180 mg, 98.1%) as a white solid. MS m/z: 206 [M+H]⁺.

Step 3: (S)-4-phenyl-6-(3-(((2-(trifluoromethyl)pyridin-3-yl)oxy)methyl)piperidin-1-yl)pyridin-2(1H)-one: To a stirred solution of (S)-3-(piperidin-3-ylmethoxy)-2-(trifluoromethyl)pyridine hydrochloride (173 mg, 0.583 mmol, 1.2 equiv) and 6-chloro-4-phenylpyridin-2(1H)-one (100 mg, 0.486 mmol, 1.00 equiv) in dioxane (1.5 mL) were added Cs₂CO₃ (475 mg, 1.46 mmol, 3 equiv) and Pd-PEPPSI-IPentCl 2-methylpyridine (o-picoline) (40.9 mg, 0.049 mmol, 0.1 equiv). The resulting mixture was stirred for overnight at 90° C. under nitrogen atmosphere. Desired product could be detected by LCMS. The resulting mixture was extracted with EtOAc (3×30 mL). The combined organic layers were washed with water (3×30 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 0% to 100% gradient in 30 min; detector, UV 254 nm. This provided (S)-4-phenyl-6-(3-(((2-(trifluoromethyl)pyridin-3-yl)oxy)methyl)piperidin-1-yl)pyridin-2(1H)-one (44 mg, 21.07%) as a light yellow solid. ¹H NMR (400 MHZ, DMSO-d₆) δ 10.32 (s, 1H), 8.27-8.25 (m, 1H), 7.82-7.80 (m, 1H), 7.71-7.67 (m, 1H), 7.63-7.54 (m, 2H), 7.48-7.38 (m, 2H), 6.26 (s, 1H), 6.06-6.06 (m, 1H), 4.43-3.94 (m, 4H), 3.04-2.62 (m, 2H), 2.08-2.07 (m, 1H), 1.91-1.88 (m, 1H), 1.74-1.71 (m, 1H), 1.61-1.34 (m, 2H). MS m/z: 430.3[M+H]⁺.

### (S)-1-(2,2-difluoroethyl)-6-(3-(((2-(trifluoromethyl)pyridin-3-yl)oxy)methyl)piperidin-1-yl)-1,5-dihydro-4H-pyrazolo[4,3-c]pyridin-4-one (306)

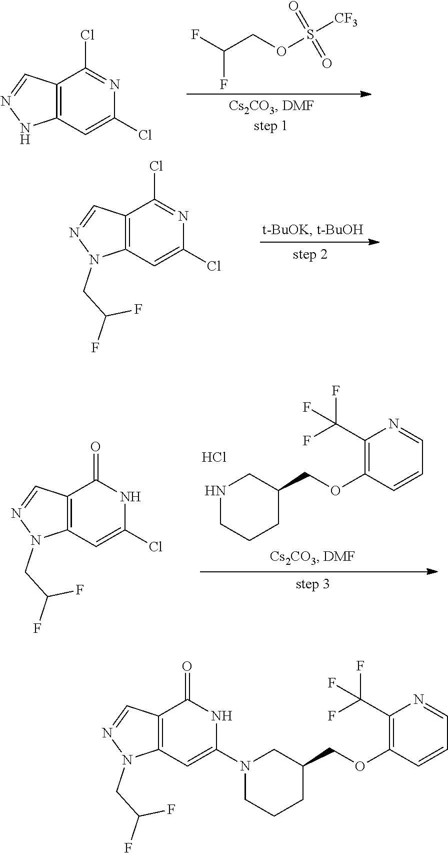

Step 1: 4,6-dichloro-1-(2,2-difluoroethyl)-1H-pyrazolo[4,3-c]pyridine: To a stirred solution of 4,6-dichloro-1H-pyrazolo[4,3-c]pyridine (300 mg, 1.596 mmol, 1 equiv) and Cs₂CO₃ (1040 mg, 3.19 mmol, 2 equiv) in DMF (5 mL) was added 2,2-difluoroethyl trifluoromethanesulfonate (512 mg, 2.40 mmol, 1.5 equiv) dropwise at 0° C. The resulting mixture was stirred for 2 h at room temperature. Desired product could be detected by LCMS. The residue was purified by reverse flash chromatography with the following conditions: column, C18 silica gel; mobile phase, MeCN in Water (0.1% FA), 0% to 100% gradient in 30 min; detector, UV 254 nm. This provided 4,6-dichloro-1-(2,2-difluoroethyl)-1H-pyrazolo[4,3-c]pyridine (300 mg, 74.6%) as a white solid. MS m/z: 252 [M+H]⁺.

Step 2: 6-chloro-1-(2,2-difluoroethyl)-1,5-dihydro-4H-pyrazolo[4,3-c]pyridin-4-one: A solution of 4,6-dichloro-1-(2,2-difluoroethyl)-1H-pyrazolo[4,3-c]pyridine (200 mg, 1.064 mmol, 1 equiv) and t-BuOK (358 mg, 3.19 mmol, 3 equiv) in t-BuOH (2 mL) was stirred for overnight at 100° C. Desired product could be detected by LCMS. The residue was purified by reverse flash chromatography with the following conditions: column, C18 silica gel; mobile phase, MeCN in Water (0.1% NH₃:H₂O), 0% to 100% gradient in 30 min; detector, UV 254 nm. This provided 6-chloro-1-(2,2-difluoroethyl)-1,5-dihydro-4H-pyrazolo[4,3-c]pyridin-4-one (150 mg, 83.2%) as a white solid. MS m/z: 234 [M+H]⁺.

Step 3: (S)-1-(2,2-difluoroethyl)-6-(3-(((2-(trifluoromethyl)pyridin-3-yl)oxy)methyl)piperidin-1-yl)-1,5-dihydro-4H-pyrazolo[4,3-c]pyridin-4-one: To a stirred mixture of 6-chloro-1-(2,2-difluoroethyl)-1,5-dihydro-4H-pyrazolo[4,3-c]pyridin-4-one(40 mg. 0.171 mmol, 1 equiv) and (S)-3-(piperidin-3-ylmethoxy)-2-(trifluoromethyl)pyridine hydrochloride (55.9 mg, 0.188 mmol, 1.1 equiv) in dioxane (2 mL) were added Cs₂CO₃ (167 mg, 0.513 mmol, 3 equiv) and Pd-PEPPSI-IPentCl 2-methylpyridine (o-picoline (14.4 mg, 0.017 mmol, 0.1 equiv) at room temperature under air atmosphere. The resulting mixture was stirred for overnight at 100° C. under nitrogen atmosphere. Desired product could be detected by LCMS. The residue was purified by Prep-TLC (CH₂Cl₂/MeOH 12:1) to afford 1-(2,2-difluoroethyl)-6-[(3S)-3-({[2-(trifluoromethyl)pyridin-3-yl]oxy}methyl)piperidin-1-yl]-5H-pyrazolo[4,3-c]pyridin-4-one (25.9 mg, 18.6%) as a light yellow oil. The residue was purified by reverse flash chromatography with the following conditions: column, C18 silica gel; mobile phase, MeCN in Water (0.1% FA), 0% to 100% gradient in 30 min; detector, UV 254 nm. This provided (S)-1-(2,2-difluoroethyl)-6-(3-(((2-(trifluoromethyl)pyridin-3-yl)oxy)methyl)piperidin-1-yl)-1,5-dihydro-4H-pyrazolo[4,3-c]pyridin-4-one (25.9 mg, 18.6%) as a white solid. ¹H NMR (400 MHZ, DMSO-d₆) δ 10.78 (s, 1H), 8.27-8.26 (m, 1H), 7.90 (s, 1H), 7.83-7.81 (m, 1H), 7.72-7.69 (m, 1H), 6.55-6.17 (m, 1H), 5.85 (s, 1H), 4.71-4.55 (m, 2H), 4.23-3.99 (m, 2H), 3.66-3.63 (m, 2H), 2.78-2.68 (m, 2H), 2.14 (s. 1H), 1.88-1.85 (m, 1H), 1.77-1.74 (m, 1H), 164-1.58 (m, 1H), 1.42-1.36 (m, 1H). MS m/z: 458.2 [M+H]⁺.

### (1H-Indol-6-yl)(4-((5-(trifluoromethyl)pyridin-3-yl)oxy)piperidin-1-yl)methanone (307)

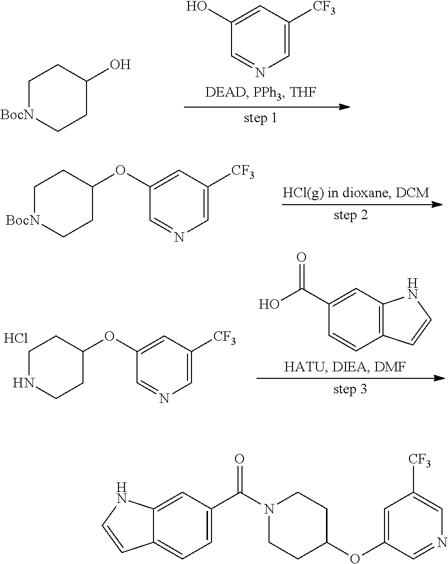

Step 1: tert-butyl 4-((5-(trifluoromethyl)pyridin-3-yl)oxy)piperidine-1-carboxylate: To a stirred mixture of tert-butyl 4-hydroxypiperidine-1-carboxylate (200 mg, 1.29 mmol, 1 equiv), 5-(trifluoromethyl)pyridin-3-ol (164 mg, 2.59 mmol, 2 equiv) and PPh₃ (390 mg, 1.55 mmol, 1.5 equiv) in THF (3 mL) was added TMAD (260 mg, 1.55 mmol, 1.2 equiv) in portions at 0° C. The resulting mixture was warmed to 50° C. and stirred for overnight. Desired product could be detected by LCMS. The resulting mixture was concentrated under reduced pressure. The residue was purified by silica gel column chromatography, eluted with EtOAc/PE (1/1) to afford tert-butyl 4-((5-(trifluoromethyl)pyridin-3-yl)oxy)piperidine-1-carboxylate (100 mg, 22.3%) as a yellow solid MS m/z): 347 [M+H]⁺.

Step 2: 3-(piperidin-4-yloxy)-5-(trifluoromethyl)pyridine hydrochloride: Followed General Procedure B using tert-butyl 4-((5-(trifluoromethyl)pyridin-3-yl)oxy)piperidine-1-carboxylate (100 mg, 0.485 mmol, 1 equiv) to afford the crude product 3-(piperidin-4-yloxy)-5-(trifluoromethyl)pyridine hydrochloride (90 mg) was used for next step without further purification. MS m/z: 247 [M+H]⁺.

Step 3. (1H-indol-6-yl)(4-((5-(trifluoromethyl)pyridin-3-yl)oxy)piperidin-1-yl)methanone: Followed General Procedure E using 3-(piperidin-4-yloxy)-5-(trifluoromethyl)pyridine hydrochloride (40 mg, 0.363 mmol, 1.00 equiv) and 1H-indole-6-carboxylic acid (80.0 mg, 0.544 mmol, 1.50 equiv) in DMF (2.00 mL). The crude product was purified by reverse flash chromatography with the following conditions: column, C18 silica gel; mobile phase, ACN in water, 10% to 50% gradient in 10 min; detector, UV 254 nm. This provided (1H-indol-6-yl)(4-((5-(trifluoromethyl)pyridin-3-yl)oxy)piperidin-1-yl)methanone (50.9 mg, 59.5%) as a white solid. ¹H NMR (400 MHZ, DMSO-d₆) δ 11.28 (s, 1H), 8.64 (d, J=2.7 Hz, 1H), 8.55 (s, 1H), 7.87 (t, J=2.3 Hz, 1H), 7.58 (d, J=8.1 Hz, 1H), 7.46 (dd, J=5.5, 2.4 Hz, 2H), 7.06 (dd, J=8.1, 1.4 Hz, 1H), 6.48 (t, J=2.5 Hz, 1H), 5.00-4.89 (m, 1H), 3.45-3.36 (m, 2H), 3.33 (s, 3H), 2.03-1.97 (m, 2H), 1.67 (s, 2H), 1.22 (dd, J=16.9, 4.9 Hz, 1H). MS m/z: 390 [M+H]⁺.

### 1-(2,2-Difluoroethyl)-6-(4-(((3-(trifluoromethyl)pyridin-4-yl)oxy)methyl)piperidin-1-yl)-1H-pyrazolo[3,4-b]pyrazine (308)

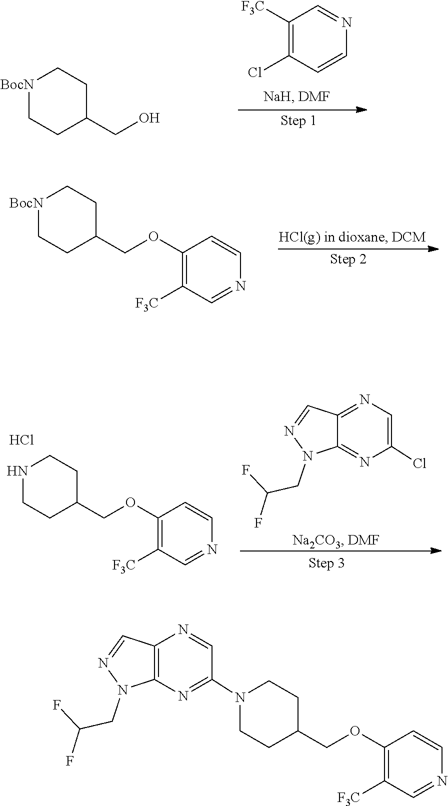

Step 1: tert-butyl 4-(((3-(trifluoromethyl)pyridin-4-yl)oxy)methyl)piperidine-1-carboxylate: Followed General Procedure D using tert-butyl 4-(hydroxymethyl)piperidine-1-carboxylate (100 mg, 0.465 mmol, 1 equiv) and 4-chloro-3-(trifluoromethyl)pyridine (92 mg, 0.55 mmol, 1.2 equiv). The crude product was purified by reverse flash chromatography with the following conditions: column, C18 silica gel; mobile phase, MeCN in Water (0.1% NH₃·H₂O), 10% to 100% gradient in 20 min; detector, UV 254 nm. This provided tert-butyl 4-(((3-(trifluoromethyl)pyridin-4-yl)oxy)methyl)piperidine-1-carboxylate (150 mg, 89.6%) as a white solid. MS m/z: 361 [M+H]⁺.

Step 2: 4-(piperidin-4-ylmethoxy)-3-(trifluoromethyl)pyridine hydrochloride: Followed General Procedure B using tert-butyl 4-(((3-(trifluoromethyl)pyridin-4-yl)oxy)methyl)piperidine-1-carboxylate (150 mg, 0.416 mmol, 1 equiv) to afford the crude product 4-(piperidin-4-ylmethoxy)-2-(trifluoromethyl)pyridine hydrochloride (100 mg, crude) as a white solid. MS m/z: 261 [M+H]⁺.

Step 3: 1-(2,2-difluoroethyl)-6-(4-(((3-(trifluoromethyl)pyridin-4-yl)oxy)methyl)piperidin-1-yl)-1H-pyrazolo[3,4-b]pyrazine: Followed General Procedure C using 4-(piperidin-4-ylmethoxy)-2-(trifluoromethyl)pyridine hydrochloride (89.5 mg, 0.274 mmol, 1.1 equiv) and 6-chloro-1-(2,2-difluoroethyl)-1H-pyrazolo[3,4-b]pyrazine (60 mg, 0.274 mmol, 1.00 equiv). The crude product was purified by reverse flash chromatography with the following conditions: column, C18 silica gel; mobile phase, MeCN in Water (0.1% NH₃·H₂O), 10% to 100% gradient in 30 min; detector, UV 254 nm. This provided 1-(2,2-difluoroethyl)-6-(4-(((3-(trifluoromethyl)pyridin-4-yl)oxy)methyl)piperidin-1-yl)-1H-pyrazolo[3,4-b]pyrazine (58.0 mg, 51.6%) as a white solid. ¹H NMR (400 MHZ, DMSO-d₆) δ 8.72-8.66 (m, 2H), 8.48 (s, 1H), 8.13 (s, 1H), 7.34 (d, J=5.9 Hz, 1H), 6.43 (tt, J=54.9, 3.8 Hz, 1H), 4.69 (td, J=15.0, 3.8 Hz, 2H), 4.61 (d, J=13.2 Hz, 2H), 4.14 (d, J=6.2 Hz, 2H), 3.13-3.01 (m, 2H), 2.28-2.11 (m, 1H), 1.92-1.83 (m, 2H), 1.45-1.31 (m, 2H). MS m/z: 433.05 [M+H]⁺.

### 1-(2,2-Difluoroethyl)-6-(4-((6-(trifluoromethyl)pyridin-2-yl)oxy)piperidin-1-yl)-1H-pyrazolo[3,4-b]pyrazine (309)

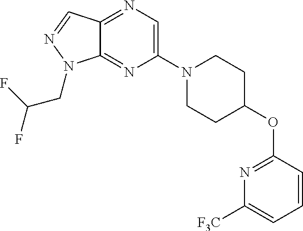 [SMILES: FC(F)Cn1ncc2ncc(N3CCC(Oc4cccc(C(F)(F)F)n4)CC3)nc21]

Followed General Procedure C using 2-(piperidin-4-yloxy)-6-(trifluoromethyl)pyridine hydrochloride (80 mg, 0.325 mmol, 1.00 equiv) and 6-chloro-1-(2,2-difluoroethyl)-1H-pyrazolo[3,4-b]pyrazine (70.8 mg, 0.325 mmol, 1.00 equiv). The crude product was purified by reverse flash chromatography with the following conditions (column, C18 gel; mobile phase, B phase: MeCN, A phase: water; 5% to 95% B gradient in 20 min; detector: UV 254/220 nm). This provided 1-(2,2-difluoroethyl)-6-(4-((6-(trifluoromethyl)pyridin-2-yl)oxy)piperidin-1-yl)-1H-pyrazolo[3,4-b]pyrazine (60 mg, 43.1%) as a white solid. ¹H NMR (400 MHZ, DMSO-d₆) δ 8.51 (s, 1H), 8.15 (s, 1H), 7.99 (t, 1H), 7.49 (d, J=7.3 Hz, 1H), 7.15 (d, J=8.4 Hz, 1H), 6.45 (tt, J=54.9, 3.8 Hz, 1H), 5.35-5.25 (m, 1H), 4.71 (td, J=15.0, 3.8 Hz, 2H), 4.22-4.11 (m, 2H), 3.71-3.60 (m, 2H), 2.19-2.05 (m, 2H), 1.84-1.71 (m, 2H). MS m/z: 429.2 [M+H]⁺.

### (1H-Indol-6-yl)(4-((4-(trifluoromethyl)pyridin-3-yl)oxy)piperidin-1-yl)methanone (310)

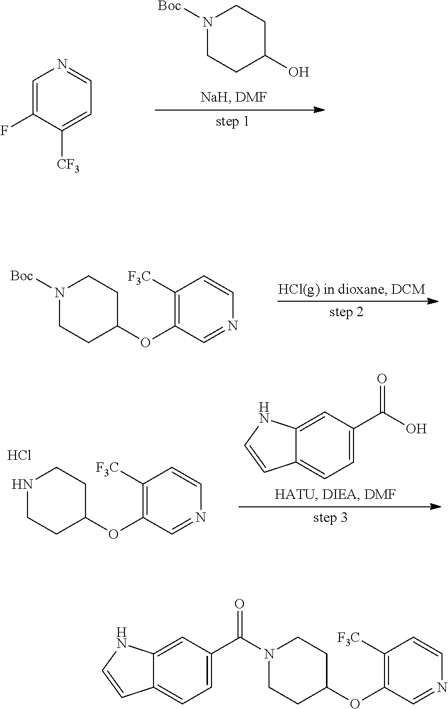

Step 1: tert-butyl 4-((4-(trifluoromethyl)pyridin-3-yl)oxy)piperidine-1-carboxylate: Followed General Procedure D using tert-butyl 4-hydroxypiperidine-1-carboxylate (100 mg, 0.497 mmol, 1.00 equiv) and ethyl 3-fluoro-4-(trifluoromethyl)pyridine (90.2 mg, 0.547 mmol, 1.10 equiv). The crude product was purified by reverse flash chromatography with the following conditions: column, C18 silica gel; mobile phase, MeCN in Water (0.1% FA), 5% to 100% gradient in 20 min; detector, UV 254 nm. This provided tert-butyl 4-((4-(trifluoromethyl)pyridin-3-yl)oxy)piperidine-1-carboxylate (85.0 mg, 49.4%) as a white solid. MS m/z: 347 [M+H]⁺.

Step 2: 3-(piperidin-4-yloxy)-4-(trifluoromethyl)pyridine hydrochloride: Followed General Procedure B using tert-butyl 4-((4-(trifluoromethyl)pyridin-3-yl)oxy)piperidine-1-carboxylate (85.0 mg, 0.245 mmol, 1.00 equiv) to afford the crude product 5-(piperidin-4-yloxy)-2-(trifluoromethyl)pyridine hydrochloride (50.0 mg) was used for next step without further purification. MS m/z: 247 [M+H]⁺.

Step 3: (1H-indol-6-yl)(4-((4-(trifluoromethyl)pyridin-3-yl)oxy)piperidin-1-yl)methanone: Followed General Procedure E using 5-(piperidin-4-yloxy)-2-(trifluoromethyl)pyridine hydrochloride (50.0 mg, 0.203 mmol, 1.00 equiv) and 1H-indole-6-carboxylic acid (39.3 mg, 0.244 mmol, 1.20 equiv). The crude product was purified by reverse flash chromatography with the following conditions: column, C18 silica gel; mobile phase, MeCN in water, 5% to 100% gradient in 20 min; detector, UV 254 nm. This provided (1H-indol-6-yl)(4-((4-(trifluoromethyl)pyridin-3-yl)oxy)piperidin-1-yl)methanone (39.8 mg, 50.0%) as a white solid. ¹H NMR (400 MHZ, DMSO-d₆) δ 11.28 (s, 1H), 8.81 (s, 1H), 8.43-8.37 (m, 1H), 7.65 (m, J=5.1, 2.3 Hz, 1H), 7.58 (d, J=8.1 Hz, 1H), 7.47 (m, J=7.2, 4.5 Hz, 2H), 7.06 (m, J=8.2, 1.2 Hz, 1H), 6.48 (m, J=2.9, 1.9, 0.9 Hz, 1H), 5.11 (m, J=7.1, 3.3 Hz, 1H), 3.53 (s, 2H), 3.37-3.31 (m, 2H), 3.33 (s, 1H), 2.03 (s, 2H), 1.73 (s, 2H). MS m/z: 390.2 [M+H]⁺.

### 1-(2,2-Difluoroethyl)-6-(4-(((4-(trifluoromethyl)pyridin-3-yl)oxy)methyl)piperidin-1-yl)-1H-pyrazolo[3,4-d]pyrimidine (311)

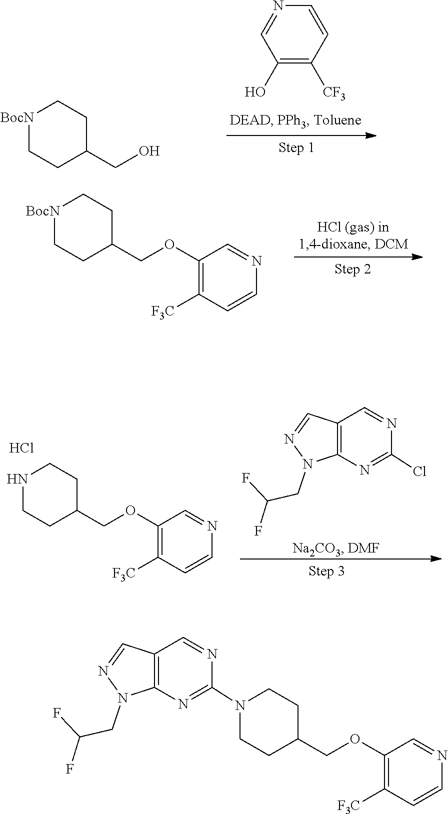

Step 1: tert-butyl 4-(((4-(trifluoromethyl)pyridin-3-yl)oxy)methyl)piperidine-1-carboxylate: A solution of tert-butyl 4-(hydroxymethyl)piperidine-1-carboxylate (238 mg, 1.11 mmol, 1.5 equiv), PPh₃ (309 mg, 1.18 mmol, 1.6 equiv), DEAD (205 mg, 1.18 mmol, 1.6 equiv) and 4-(trifluoromethyl)pyridin-3-ol (120 mg, 0.74 mmol, 1 equiv) in Toluene (3 mL) was stirred for overnight at 100° C. under air atmosphere. The reaction was monitored by LCMS. The residue was purified by reverse flash chromatography with the following conditions: column, C18 silica gel; mobile phase, MeCN in Water (0.1% FA), 0% to 100% gradient in 30 min; detector, UV 254 nm. The resulting mixture was concentrated under reduced pressure. This provided tert-butyl 4-(((4-(trifluoromethyl)pyridin-3-yl)oxy)methyl)piperidine-1-carboxylate (247 mg, 93.2%) as a yellow oil. MS m/z: 361 [M+H]⁺.

Step 2: 3-(piperidin-4-ylmethoxy)-4-(trifluoromethyl)pyridine hydrochloride: Followed General Procedure B using tert-butyl 4-(((4-(trifluoromethyl)pyridin-3-yl)oxy)methyl)piperidine-1-carboxylate (247 mg, 0.69 mmol, 1 equiv) to afford the crude product 3-(piperidin-4-ylmethoxy)-4-(trifluoromethyl)pyridine hydrochloride (100 mg). MS m/z: 261 [M+H]⁺.

Step 3: 1-(2,2-difluoroethyl)-6-(4-(((4-(trifluoromethyl)pyridin-3-yl)oxy)methyl)piperidin-1-yl)-1H-pyrazolo[3,4-d]pyrimidine: Followed General Procedure C using 6-chloro-1-(2,2-difluoroethyl)-1H-pyrazolo[3,4-d]pyrimidine (101 mg, 0.46 mmol, 1.2 equiv) and 3-(piperidin-4-ylmethoxy)-4-(trifluoromethyl)pyridine hydrochloride (100 mg, 0.39 mmol, 1.00 equiv). The crude product was purified by reverse flash chromatography with the following conditions: column, C18 silica gel; mobile phase, MeCN in Water (0.1% FA), 0% to 100% gradient in 30 min; detector, UV 254 nm. This provided 1-(2,2-difluoroethyl)-6-(4-(((4-(trifluoromethyl)pyridin-3-yl)oxy)methyl)piperidin-1-yl)-1H-pyrazolo[3,4-d]pyrimidine (39 mg, 22.8%) as a white solid. ¹H NMR (400 MHZ, DMSO-d₆) δ 8.69 (s, 1H), 8.48 (s, 1H), 8.41 (d, J=4.8 Hz, 1H), 8.13 (s, 1H), 7.64 (d, J=4.9 Hz, 1H), 6.60-6.26 (m, 1H), 4.76-4.65 (m, 2H), 4.61 (d, J=13.3 Hz, 2H), 4.19 (d, J=6.2 Hz, 2H), 3.14-3.01 (m, 2H), 2.17 (d, J=4.6 Hz, 1H), 1.89 (d, J=13.0 Hz, 2H), 1.48-1.32 (m, 2H). MS m/z: 443.05 [M+H]⁺.

### 1-(2,2-Difluoroethyl)-6-(4-(((5-(trifluoromethyl)pyridin-3-yl)oxy)methyl)piperidin-1-yl)-1H-pyrazolo[3,4-b]pyrazine (312)

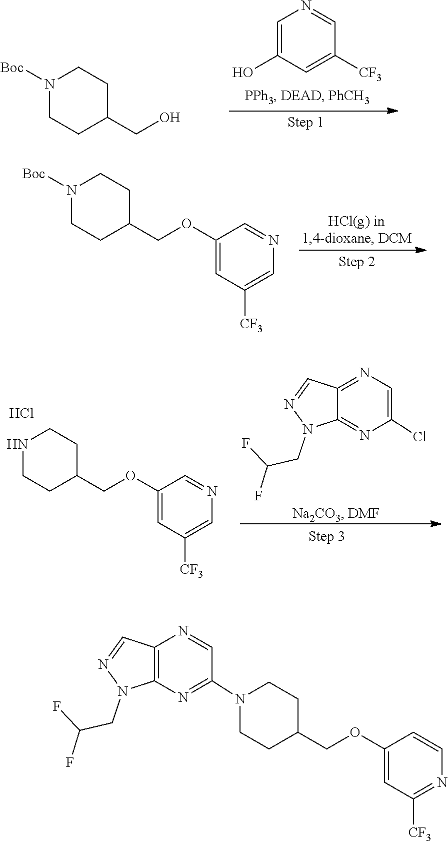

Step 1: tert-butyl 4-(((5-(trifluoromethyl)pyridin-3-yl)oxy)methyl)piperidine-1-carboxylate: To a stirred mixture of 3-(piperidin-4-ylmethoxy)-5-(trifluoromethyl)pyridine hydrochloride (198 mg, 0.920 mmol, 1 equiv) in Toluene (1 mL) were added 5-(trifluoromethyl)pyridin-3-ol (150 mg, 0.920 mmol, 1.00 equiv) and PPh₃ (362 mg, 1.38 mmol, 1.5 equiv) and DEAD (240 mg, 1.38 mmol, 1.5 equiv) in portions at 0° C. under air atmosphere. The resulting mixture was stirred for overnight at 100° C. under air atmosphere. The resulting mixture was concentrated under reduced pressure. The residue was purified by silica gel column chromatography, eluted with PE/EtOAc (0% to 100% gradient in 20 min) to afford tert-butyl 4-(((5-(trifluoromethyl)pyridin-3-yl)oxy)methyl)piperidine-1-carboxylate (256 mg, 77.2%) as a light yellow solid. MS m/z: 361 [M+H]⁺.

Step 2: 3-(piperidin-4-ylmethoxy)-5-(trifluoromethyl)pyridine hydrochloride: Followed General Procedure B using tert-butyl tert-butyl 4-(((5-(trifluoromethyl)pyridin-3-yl)oxy)methyl)piperidine-1-carboxylate (200 mg, 0.55 mmol, 1 equiv) to afford the crude product 3-(piperidin-4-ylmethoxy)-5-(trifluoromethyl)pyridine hydrochloride (123 mg) as a light yellow solid. MS m/z: 297 [M+H]⁺.

Step 3: 1-(2,2-difluoroethyl)-6-(4-(((5-(trifluoromethyl)pyridin-3-yl)oxy)methyl)piperidin-1-yl)-1H-pyrazolo[3,4-b]pyrazine: Followed General Procedure C using 3-(piperidin-4-ylmethoxy)-5-(trifluoromethyl)pyridine hydrochloride (123 mg, 0.416 mmol, 1.3 equiv) and 6-chloro-1-(2,2-difluoroethyl)-1H-pyrazolo[3,4-b]pyrazine (70 mg, 0.320 mmol, 1 equiv). The crude product was purified by silica gel column chromatography, eluted with CH₃CN/H₂O (60% to 90% gradient in 15 min) to afford 1-(2,2-difluoroethyl)-6-(4-(((5-(trifluoromethyl)pyridin-3-yl)oxy)methyl)piperidin-1-yl)-1H-pyrazolo[3,4-b]pyrazine (112 mg, 78.9%) as a white solid. ¹H NMR (400 MHZ, DMSO-d₆) δ 8.61 (d, J=2.8 Hz, 1H), 8.54 (s, 1H), 8.48 (s, 1H), 8.13 (s, 1H), 7.77 (t, J=2.4 Hz, 1H), 6.44 (m, 1H), 4.80-4.50 (m, 4H), 4.08 (d, J=6.4 Hz, 2H), 3.08 (t, J=12.4 Hz, 2H), 2.17 (s, 1H), 1.92 (d, J=12.7 Hz, 2H), 1.47-1.30 (m, 2H). MS m/z: 443.15 [M+H]⁺.

### 1-(2,2-Difluoroethyl)-6-(4-(((3-(trifluoromethyl)pyridin-2-yl)oxy)methyl)piperidin-1-yl)-1H-pyrazolo[3,4-b]pyrazine (313)

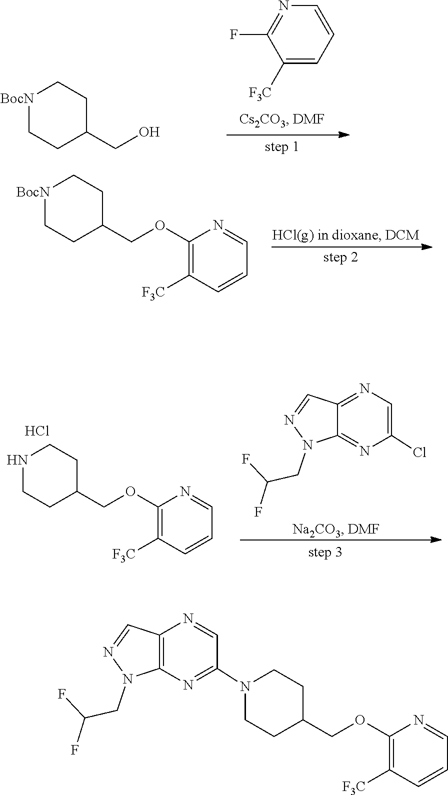

Step 1: tert-butyl 4-(((3-(trifluoromethyl)pyridin-2-yl)oxy)methyl)piperidine-1-carboxylate: To a stirred mixture of tert-butyl 4-(hydroxymethyl)piperidine-1-carboxylate (100 mg, 0.464 mmol, 1.00 equiv) and NaH (55.7 mg, 1.39 mmol, 3.00 equiv, 60%) in DMF (1.5 mL) was added 2-fluoro-3-(trifluoromethyl)pyridine (84.4 mg, 0.510 mmol, 1.10 equiv) at 0° C. under N₂ atmosphere. The resulting mixture was stirred for 2 h at room temperature. Desired product could be detected by LCMS. The reaction was quenched with water at 0° C. The resulting mixture was extracted with EtOAc (3×30 mL). The combined organic layers were washed with water (2×10 mL) and brine (1×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 5% to 100% gradient in 20 min; detector, UV 254 nm. This provided tert-butyl 4-(((3-(trifluoromethyl)pyridin-2-yl)oxy)methyl)piperidine-1-carboxylate (85 mg, 50.8%) as a white solid. MS m/z: 361 [M+H]⁺.

Step 2: 2-(piperidin-4-ylmethoxy)-3-(trifluoromethyl)pyridine hydrochloride: Followed General Procedure B using tert-butyl 4-(((3-(trifluoromethyl)pyridin-2-yl)oxy)methyl)piperidine-1-carboxylate (85 mg, 0.235 mmol, 1.00 equiv) to afford the crude product 2-(piperidin-4-ylmethoxy)-5-(trifluoromethyl)pyridine hydrochloride (80 mg) was used for next step without further purification. MS m/z: 261 [M+H]⁺.

Step 3: 1-(2,2-difluoroethyl)-6-(4-(((5-(trifluoromethyl)pyridin-2-yl)oxy)methyl)piperidin-1-yl)-1H-pyrazolo[3,4-b]pyrazine: To a stirred solution of 2-(piperidin-4-ylmethoxy)-5-(trifluoromethyl)pyridine hydrochloride (81.5 mg, 0.274 mmol, 1.00 equiv) and 6-chloro-1-(2,2-difluoroethyl)-1H-pyrazolo[3,4-b]pyrazine (60.0 mg, 0.274 mmol, 1.00 equiv) in DMF (1.00 mL) was added Na₂CO₃ (87.3 mg, 0.822 mmol, 3.00 equiv). The resulting mixture was stirred for 2 hours at 100° C. The residue was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(4-(((5-(trifluoromethyl)pyridin-2-yl)oxy)methyl)piperidin-1-yl)-1H-pyrazolo[3,4-b]pyrazine (60 mg, 50.1%) as a white solid. ¹H NMR (400 MHZ, DMSO-d₆) δ 8.51-8.41 (m, 2H), 8.18-8.05 (m, 2H), 7.24-7.11 (m, 1H), 6.58-6.29 (m, 1H), 4.82-4.52 (m, 4H), 4.31-4.30 (m, 2H), 3.08-3.01 (m, 2H), 2.23-2.07 (m, 1H), 1.89-1.86 (m, 2H), 1.42-1.23 (m, 2H). MS m/z: 443.4 [M+H]⁺.

### (1H-Indol-6-yl)(4-((6-(trifluoromethyl)pyridin-3-yl)oxy)piperidin-1-yl)methanone (314)

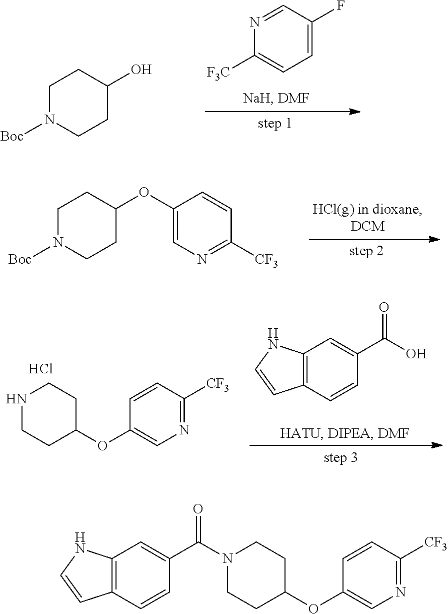

Step 1: tert-butyl 4-((6-(trifluoromethyl)pyridin-3-yl)oxy)piperidine-1-carboxylate: Followed General Procedure D using tert-butyl 4-hydroxypiperidine-1-carboxylate (100 mg, 0.497 mmol, 1.00 equiv) and ethyl 5-fluoro-2-(trifluoromethyl)pyridine (90.2 mg, 0.547 mmol, 1.10 equiv). The crude product was purified by reverse flash chromatography with the following conditions: column, C18 silica gel; mobile phase, MeCN in Water (0.1% FA), 5% to 100% gradient in 20 min; detector, UV 254 nm. This provided ethyl tert-butyl 4-((6-(trifluoromethyl)pyridin-3-yl)oxy)piperidine-1-carboxylate (85.0 mg, 49.4%) as a white solid. MS m/z: 347 [M+H]⁺.

Step 2: 5-(piperidin-4-yloxy)-2-(trifluoromethyl)pyridine hydrochloride: Followed General Procedure B using tert-butyl 4-((6-(trifluoromethyl)pyridin-3-yl)oxy)piperidine-1-carboxylate (85.0 mg, 0.245 mmol, 1.00 equiv) to afford the crude product 5-(piperidin-4-yloxy)-2-(trifluoromethyl)pyridine hydrochloride (50.0 mg). MS m/z: 247 [M+H]⁺.

Step 3: (1H-indol-6-yl)(4-((6-(trifluoromethyl)pyridin-3-yl)oxy)piperidin-1-yl)methanone: Followed General Procedure E using 5-(piperidin-4-yloxy)-2-(trifluoromethyl)pyridine (50.0 mg, 0.203 mmol, 1.00 equiv) and 1H-indole-6-carboxylic acid (39.3 mg, 0.244 mmol, 1.20 equiv) in DMF (3.00 mL). The crude product was purified by reverse flash chromatography with the following conditions: column, C18 silica gel; mobile phase, MeCN in water, 5% to 100% gradient in 20 min; detector, UV 254 nm. This provided (1H-indol-6-yl)(4-((6-(trifluoromethyl)pyridin-3-yl)oxy)piperidin-1-yl)methanone (39.8 mg, 50.0%) as a white solid. ¹H NMR (400 MHZ, DMSO-d₆) δ 11.27 (s, 1H), 8.48 (d, J=2.8 Hz, 1H), 7.84 (d, J=8.7 Hz, 1H), 7.70 (dd, J=8.8, 2.9 Hz, 1H), 7.58 (d, J=8.1 Hz, 1H), 7.50-7.43 (m, 2H), 7.05 (dd, J=8.1, 1.5 Hz, 1H), 6.51-6.45 (m, 1H), 4.91 (t, J=3.9 Hz, 1H), 3.86 (s, 2H), 3.51 (s, 2H), 2.03 (s, 2H), 1.69 (s, 2H). MS m/z: 390.1 [M+H]⁺.

### 1-(2,2-Difluoroethyl)-6-(4-(((2-(trifluoromethyl)pyridin-4-yl)oxy)methyl)piperidin-1-yl)-1H-pyrazolo[3,4-b]pyrazine (315)

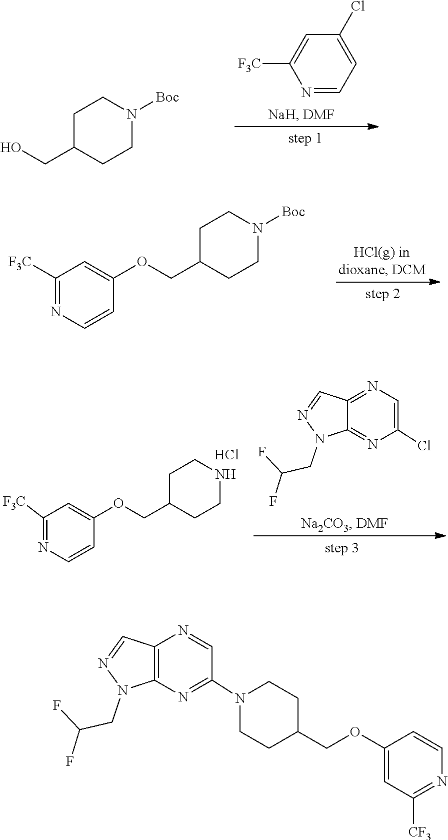

Step 1: tert-butyl 4-(((2-(trifluoromethyl)pyridin-4-yl)oxy)methyl)piperidine-1-carboxylate: Following General Procedure D using tert-butyl 4-(hydroxymethyl)piperidine-1-carboxylate (200 mg, 0.929 mmol, 1 equiv) and 4-chloro-2-(trifluoromethyl)pyridine (184 mg, 1.11 mmol, 1.2 equiv). The crude product was purified by reverse flash chromatography with the following conditions: column, C18 silica gel; mobile phase, MeCN in Water (0.1% NH₃·H₂O), 10% to 100% gradient in 20 min; detector, UV 254 nm. This provided tert-butyl 4-(((2-(trifluoromethyl)pyridin-4-yl)oxy)methyl)piperidine-1-carboxylate (220 mg, 65.7%) as a white solid. MS m/z: 361 [M+H]⁺.

Step 2: 4-(piperidin-4-ylmethoxy)-2-(trifluoromethyl)pyridine hydrochloride: Followed General Product B using tert-butyl 4-(((2-(trifluoromethyl)pyridin-4-yl)oxy)methyl)piperidine-1-carboxylate (220 mg, 0.610 mmol, 1 equiv) to afford the crude product 4-(piperidin-4-ylmethoxy)-2-(trifluoromethyl)pyridine hydrochloride (170 mg) as a white solid. MS m/z: 261 [M+H]⁺.

Step 3: 1-(2,2-difluoroethyl)-6-(4-(((2-(trifluoromethyl)pyridin-4-yl)oxy)methyl)piperidin-1-yl)-1H-pyrazolo[3,4-b]pyrazine: Followed General Procedure C using 4-(piperidin-4-ylmethoxy)-2-(trifluoromethyl)pyridine hydrochloride (81.4 mg, 0.274 mmol, 1 equiv) and 6-chloro-1-(2,2-difluoroethyl)-1H-pyrazolo[3,4-b]pyrazine (60 mg, 0.274 mmol, 1.00 equiv). The crude product was purified by reverse flash chromatography with the following conditions: column, C18 silica gel; mobile phase, MeCN in Water (0.1% NH₃·H₂O), 10% to 100% gradient in 30 min; detector, UV 254 nm. This provided 1-(2,2-difluoroethyl)-6-(4-(((2-(trifluoromethyl)pyridin-4-yl)oxy)methyl)piperidin-1-yl)-1H-pyrazolo[3,4-b]pyrazine (63.2 mg, 51.6%) as a white solid. ¹H NMR (400 MHZ, DMSO-d₆) δ 8.56 (d, J=5.7 Hz, 1H), 8.48 (s, 1H), 8.12 (s, 1H), 7.44 (d, J=2.4 Hz, 1H), 7.32-7.25 (m, 1H), 6.64-6.26 (m, 1H), 4.75-4.65 (m, 2H), 4.60 (d, J=13.4 Hz, 2H), 4.09 (d, J=6.4 Hz, 2H), 3.07 (t, J=12.4 Hz, 2H), 2.17 (s, 1H), 1.90 (d, J=13.0 Hz, 2H), 1.47-1.27 (m, 2H). MS m/z: 433.2 [M+H]⁺.

### 1-(2,2-Difluoroethyl)-6-(4-(((6-(trifluoromethyl)pyridin-3-yl)oxy)methyl)piperidin-1-yl)-1H-pyrazolo[3,4-b]pyrazine (316)

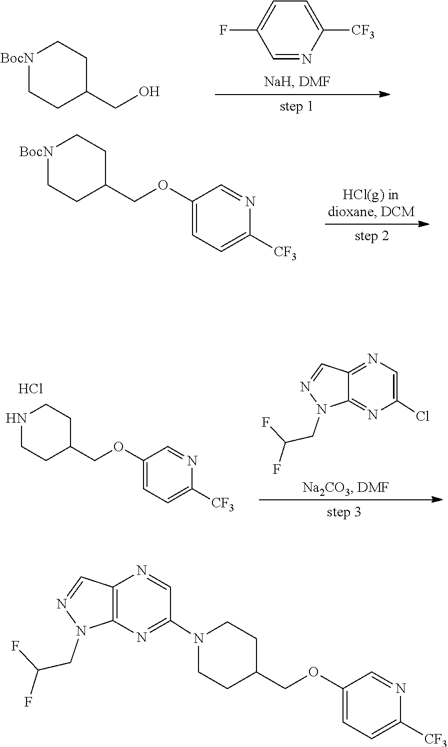

Step 1: tert-butyl 4-(((6-(trifluoromethyl)pyridin-3-yl)oxy)methyl)piperidine-1-carboxylate: Followed General Procedure D using tert-butyl 4-(hydroxymethyl)piperidine-1-carboxylate (100 mg, 0.464 mmol, 1.00 equiv) and ethyl 5-fluoro-2-(trifluoromethyl)pyridine (84.4 mg, 0.510 mmol, 1.10 equiv). The crude product was purified by reverse flash chromatography with the following conditions: column, C18 silica gel; mobile phase, MeCN in Water (0.1% FA), 5% to 100% gradient in 20 min; detector, UV 254 nm. This provided ethyl tert-butyl 4-(((6-(trifluoromethyl)pyridin-3-yl)oxy)methyl)piperidine-1-carboxylate (85 mg, 50.8%) as a white solid. MS m/z: 361 [M+H]⁺.

Step 2: 5-(piperidin-4-ylmethoxy)-2-(trifluoromethyl)pyridine hydrochloride: Followed General Procedure B using tert-butyl 4-(((6-(trifluoromethyl)pyridin-3-yl)oxy)methyl)piperidine-1-carboxylate (85 mg, 0.485 mmol, 1.00 equiv) to afford the crude product 5-(piperidin-4-ylmethoxy)-2-(trifluoromethyl)pyridine hydrochloride (50.0 mg). MS m/z: 261 [M+H]⁺.

Step 3: 1-(2,2-difluoroethyl)-6-(4-(((6-(trifluoromethyl)pyridin-3-yl)oxy)methyl)piperidin-1-yl)-1H-pyrazolo[3,4-b]pyrazine: Followed General Procedure C using 5-(piperidin-4-ylmethoxy)-2-(trifluoromethyl)pyridine (50.0 mg, 0.192 mmol, 1.00 equiv) and 6-chloro-1-(2,2-difluoroethyl)-1H-pyrazolo[3,4-b]pyrazine (46.2 mg, 0.211 mmol, 1.10 equiv). The crude product was purified by reverse flash chromatography with the following conditions (column, C18 gel; mobile phase, B phase: MeCN, A phase: water; 5% to 100% B gradient in 20 min; detector: UV 254/220 nm). This provided 1-(2,2-difluoroethyl)-6-(4-(((6-(trifluoromethyl)pyridin-3-yl)oxy)methyl)piperidin-1-yl)-1H-pyrazolo[3,4-b]pyrazine (49.0 mg, 57.6%) as a white solid. ¹H NMR (400 MHZ, DMSO-d₆) δ 8.50-8.43 (m, 2H), 8.13 (s, 1H), 7.84 (d, J=8.7 Hz, 1H), 7.62 (dd, J=8.7, 2.9 Hz, 1H), 6.61-6.26 (m, 1H), 4.76-4.57 (m, 4H), 4.07 (d, J=6.4 Hz, 2H), 3.07 (t, J=12.4 Hz, 2H), 2.24-2.11 (m, 1H), 1.95-1.88 (m, 2H), 1.44-1.31 (m, 2H). MS m/z: 443.39 [M+H]⁺.

### 1-(2,2-Difluoroethyl)-6-(4-(((5-(trifluoromethyl)pyridin-2-yl)oxy)methyl)piperidin-1-yl)-1H-pyrazolo[3,4-b]pyrazine (317)

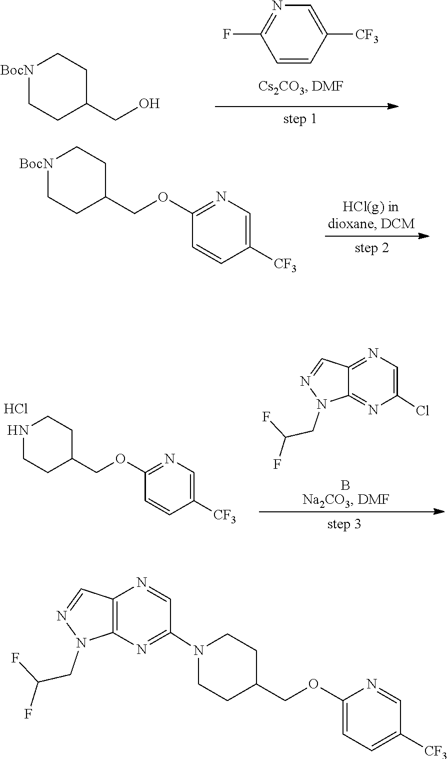

Step 1: tert-butyl 4-(((5-(trifluoromethyl)pyridin-2-yl)oxy)methyl)piperidine-1-carboxylate: A solution of tert-butyl 4-(hydroxymethyl)piperidine-1-carboxylate (300 mg. 1.39 mmol, 1.00 equiv) and 2-fluoro-5-(trifluoromethyl)pyridine (276 mg, 1.67 mmol, 1.20 equiv) and Cs₂CO₃ (1.31 g. 4.17 mmol, 3.00 equiv) in DMF (4.00 mL) was stirred for 2 hours at 100° C. The residue was purified by reverse flash chromatography with the following conditions (column, C18 gel; mobile phase, B phase: MeCN, A phase: water; 0% to 100% B gradient in 20 min; detector: UV 254/220 nm). This provided tert-butyl 4-(((5-(trifluoromethyl)pyridin-2-yl)oxy)methyl)piperidine-1-carboxylate (454 mg, 90.4%) as a yellow solid. MS m/z: 361 [M+H]⁺.

Step 2: 2-(piperidin-4-ylmethoxy)-5-(trifluoromethyl)pyridine hydrochloride: Followed General Procedure B using A tert-butyl 4-(((5-(trifluoromethyl)pyridin-2-yl)oxy)methyl)piperidine-1-carboxylate (450 mg, 1.26 mmol, 1.00 equiv) to afford the crude product 2-(piperidin-4-ylmethoxy)-5-(trifluoromethyl)pyridine hydrochloride (300 mg) was used for next step without further purification. MS m/z: 261 [M+H]⁺.

Step 3: 1-(2,2-difluoroethyl)-6-(4-(((5-(trifluoromethyl)pyridin-2-yl)oxy)methyl)piperidin-1-yl)-1H-pyrazolo[3,4-b]pyrazine: Followed General Procedure C using 2-(piperidin-4-ylmethoxy)-5-(trifluoromethyl)pyridine hydrochloride (81.5 mg, 0.274 mmol, 1.00 equiv) and 6-chloro-1-(2,2-difluoroethyl)-1H-pyrazolo[3,4-b]pyrazine (60.0 mg, 0.274 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 1-(2,2-difluoroethyl)-6-(4-(((5-(trifluoromethyl)pyridin-2-yl)oxy)methyl)piperidin-1-yl)-1H-pyrazolo[3,4-b]pyrazine (83.4 mg, 68.4%) as a white solid. ¹H NMR (400 MHZ, DMSO-d₆) δ 8.58 (s, 1H), 8.50-8.45 (m, 1H), 8.15-8.04 (m, 2H), 7.03 (d, J=8.9 Hz, 1H), 6.60-6.27 (m, 1H), 4.79-4.56 (m, 4H), 4.32-4.21 (m, 2H), 3.09-3.02 (m, 2H), 2.16 (s, 1H), 1.93-1.85 (m, 2H), 1.42-1.29 (m, 2H). MS m/z: 443.3 [M+H]⁺.

### (1H-Indol-6-yl)(4-((3-(trifluoromethyl)pyridin-4-yl)oxy)piperidin-1-yl)methanone (318)

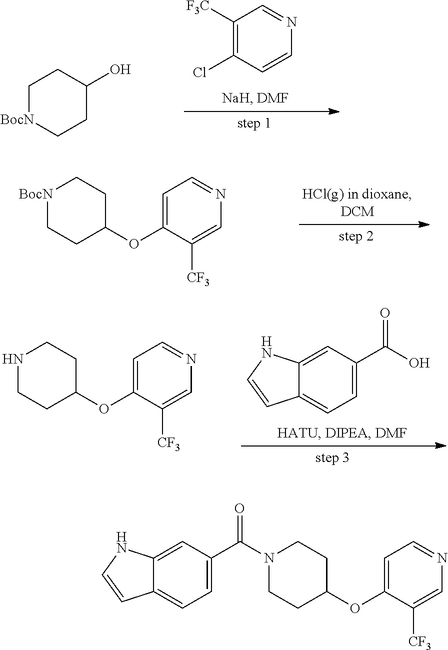

Step 1: tert-butyl 4-((3-(trifluoromethyl)pyridin-4-yl)oxy)piperidine-1-carboxylate: Followed General Procedure D using tert-butyl 4-hydroxypiperidine-1-carboxylate (100 mg, 0.497 mmol, 1 equiv) and 4-chloro-3-(trifluoromethyl)pyridine (90.20 mg, 0.497 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 4-((3-(trifluoromethyl)pyridin-4-yl)oxy)piperidine-1-carboxylate (60 mg, 34.8%) as a white solid. MS m/z: 347 [M+H]⁺.

Step 2: 4-(piperidin-4-yloxy)-3-(trifluoromethyl)pyridine hydrochloride: Followed General Procedure B using tert-butyl 4-((3-(trifluoromethyl)pyridin-4-yl)oxy)piperidine-1-carboxylate (60 mg, 0.173 mmol, 1 equiv) to afford the crude product 4-(piperidin-4-yloxy)-3-(trifluoromethyl)pyridine hydrochloride (40 mg). MS m/z: 247 [M+H]⁺.

Step 3: (1H-indol-6-yl)(4-((3-(trifluoromethyl)pyridin-4-yl)oxy)piperidin-1-yl)methanone: Followed General Procedure C using 4-(piperidin-4-yloxy)-3-(trifluoromethyl)pyridine hydrochloride (40 mg, 0.162 mmol, 1 equiv) and 1H-indole-6-carboxylic acid (28.8 mg, 0.178 mmol, 1.1 equiv). The crude product was purified by reverse flash chromatography with the following conditions: column, C18 silica gel; mobile phase, ACN in water, 10% to 95% gradient in 10 min; detector, UV 254 nm. This provided (1H-indol-6-yl)(4-((3-(trifluoromethyl)pyridin-4-yl)oxy)piperidin-1-yl)methanone (13.4 mg, 20.7%) as a white solid. ¹H NMR (400 MHZ, DMSO-d₆) δ 11.26 (s, 1H), 8.89-8.59 (m, 2H), 7.58 (d, 1H), 7.50-7.41 (m, 3H), 7.13-6.93 (m, 1H), 6.56-6.33 (m, 1H), 5.26-4.90 (m, 1H), 3.97-3.37 (m, 4H), 2.00 (s, 2H), 1.73 (s, 2H), 1.28-1.16 (m, 1H). MS m/z: 390.1 [M+H]⁺.

### (1H-Indol-6-yl)(4-((2-(trifluoromethyl)pyridin-4-yl)oxy)piperidin-1-yl)methanone (319)

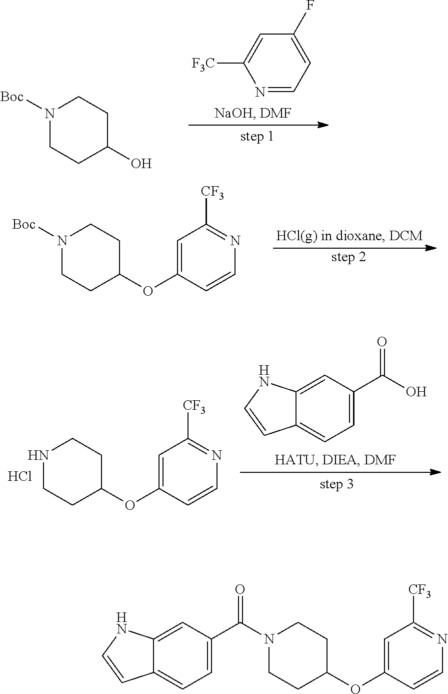

Step 1: tert-butyl 4-((2-(trifluoromethyl)pyridin-4-yl)oxy)piperidine-1-carboxylate: Followed General Procedure D using tert-butyl 4-hydroxypiperidine-1-carboxylate (100 mg, 0.497 mmol, 1 equiv) and 4-fluoro-2-(trifluoromethyl)pyridine (90.2 mg, 0.547 mmol, 1.1 equiv). The crude product was purified by reverse flash chromatography with the following conditions: column, C18 silica gel; mobile phase, MeCN in Water (0.1% NH₃·H₂O), 10% to 100% gradient in 20 min; detector, UV 254 nm. This provided tert-butyl 4-((2-(trifluoromethyl)pyridin-4-yl)oxy)piperidine-1-carboxylate (100 mg, 58.1%) as a white solid. MS m/z: 347 [M+H]⁺.

Step 2: 4-(piperidin-4-yloxy)-2-(trifluoromethyl)pyridine hydrochloride: Followed General Procedure B using tert-butyl 4-((2-(trifluoromethyl)pyridin-4-yl)oxy)piperidine-1-carboxylate (100 mg, 0.289 mmol, 1 equiv) to afford 4-(piperidin-4-yloxy)-2-(trifluoromethyl)pyridine hydrochloride (80 mg, crude) as a white solid. MS m/z: 247 [M+H]⁺.

Step 3: (1H-indol-6-yl)(4-((2-(trifluoromethyl)pyridin-4-yl)oxy)piperidin-1-yl)methanone: Followed General Procedure E using 1H-indole-6-carboxylic acid (31.3 mg, 0.195 mmol, 1.1 equiv) and 4-(piperidin-4-yloxy)-2-(trifluoromethyl)pyridine hydrochloride (50 mg, 0.177 mmol, 1 equiv). The crude product was purified by reverse flash chromatography with the following conditions: column, C18 silica gel; mobile phase, MeCN in Water (0.1% NH₃·H₂O), 10% to 100% gradient in 30 min; detector, UV 254 nm. This provided (1H-indol-6-yl)(4-((2-(trifluoromethyl)pyridin-4-yl)oxy)piperidin-1-yl)methanone (30.4 mg, 43.7%) as a white solid. ¹H NMR (400 MHZ, DMSO-d₆) § 11.27 (s, 1H), 8.56 (d, J=5.7 Hz, 1H), 7.58 (d, J=8.2 Hz, 1H), 7.52-7.43 (m, 3H), 7.38-7.31 (m, 1H), 7.12-6.99 (m, 1H), 6.53-6.43 (m, 1H), 5.06-4.93 (m, 1H), 3.86 (s, 2H), 3.42 (s, 2H), 2.05 (d, J=21.8 Hz, 2H), 1.67 (s, 2H). MS m/z: 390.2 [M+H]⁺.

### (1H-Indol-6-yl)(4-((5-(trifluoromethyl)pyridin-2-yl)oxy)piperidin-1-yl)methanone (320)

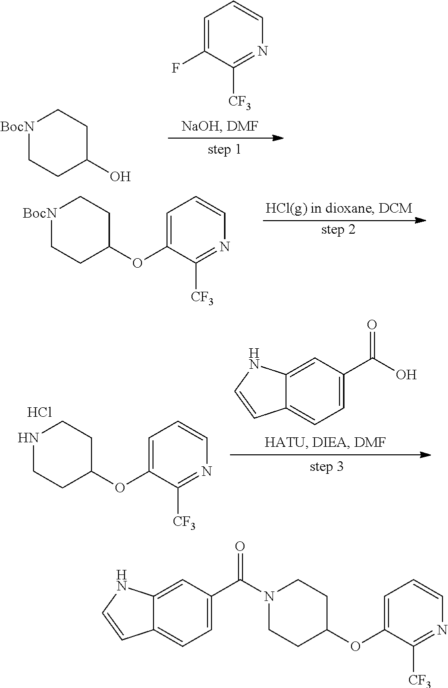

Step 1: tert-butyl 4-((2-(trifluoromethyl)pyridin-3-yl)oxy)piperidine-1-carboxylate: Followed General Procedure D using tert-butyl 4-hydroxypiperidine-1-carboxylate (100 mg, 0.50 mmol, 1 equiv) and 3-fluoro-2-(trifluoromethyl)pyridine (107 mg, 0.65 mmol, 1.3 equiv). The crude product was purified by reverse flash chromatography with the following conditions: column, C18 silica gel; mobile phase, MeCN in Water (0.1% FA), 5% to 100% gradient in 20 min; detector, UV 254 nm. This provided tert-butyl 4-((2-(trifluoromethyl)pyridin-3-yl)oxy)piperidine-1-carboxylate (100 mg, 57.6%). MS m/z: 347 [M+H]⁺.

Step 2: 3-(piperidin-4-yloxy)-2-(trifluoromethyl)pyridine hydrochloride: Followed General Procedure B using tert-butyl 4-{[2-(trifluoromethyl)pyridin-3-yl]oxy}piperidine-1-carboxylate (100 mg, 0.289 mmol, 1 equiv) to afford the crude product 3-(piperidin-4-yloxy)-2-(trifluoromethyl)pyridine hydrochloride (100 mg). MS m/z: 247 [M+H]⁺.

Step 3: (1H-indol-6-yl)(4-((2-(trifluoromethyl)pyridin-3-yl)oxy)piperidin-1-yl)methanone: Followed General Procedure E using 1H-indole-6-carboxylic acid (78.5 mg, 0.487 mmol, 1.2 equiv) and 3-(piperidin-4-yloxy)-2-(trifluoromethyl)pyridine (100 mg, 0.406 mmol, 1 equiv). The crude product was purified by reverse flash chromatography with the following conditions: column, C18 silica gel; mobile phase, MeCN in water, 10% to 100% gradient in 20 min; detector, UV 254 nm. The resulting mixture was concentrated under reduced pressure. This provided (1H-indol-6-yl)(4-((2-(trifluoromethyl)pyridin-3-yl)oxy)piperidin-1-yl)methanone (80.9 mg, 50.9%) as a white solid. ¹H NMR (400 MHZ, DMSO-d₆) δ 11.28 (s, 1H), 8.29-8.23 (m, 1H), 7.91 (d, J=8.6 Hz, 1H), 7.72-7.65 (m, 1H), 7.58 (d, J=8.1 Hz, 1H), 7.50-7.43 (m, 2H), 7.09-7.03 (m, 1H), 6.51-6.45 (m, 1H), 5.03-4.93 (m, 1H), 3.69 (s, 2H), 3.53 (d, J=15.5 Hz, 2H), 1.99 (s, 2H), 1.84-1.41 (m, 2H). MS m/z: 390.1 [M+H]⁺.

### (1H-Indol-6-yl)(4-((3-(trifluoromethyl)pyridin-2-yl)oxy)piperidin-1-yl)methanone (321)

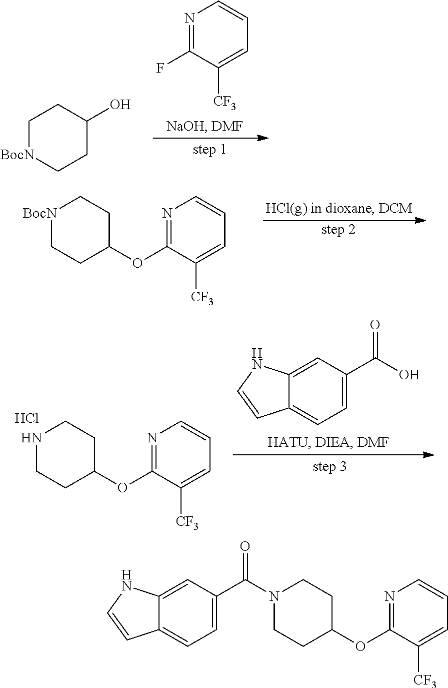

Step 1: tert-butyl 4-((3-(trifluoromethyl)pyridin-2-yl)oxy)piperidine-1-carboxylate: Followed General Procedure D using tert-butyl 4-hydroxypiperidine-1-carboxylate (100 mg, 0.497 mmol, 1 equiv) and 2-fluoro-3-(trifluoromethyl)pyridine (82 mg, 0.497 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 4-((3-(trifluoromethyl)pyridin-2-yl)oxy)piperidine-1-carboxylate (120 mg, 69.7%) as a white solid. MS m/z: 347 [M+H]⁺.

Step 2: 2-(piperidin-4-yloxy)-4-(trifluoromethyl)pyridine hydrochloride: Followed General Procedure B using tert-butyl 4-((4-(trifluoromethyl)pyridin-2-yl)oxy)piperidine-1-carboxylate (120 mg, 0.674 mmol, 1 equiv) to afford the crude product 2-(piperidin-4-yloxy)-4-(trifluoromethyl)pyridine hydrochloride (80 mg). MS m/z: 247 [M+H]⁺.

Step 3: (1H-indol-6-yl)(4-((4-(trifluoromethyl)pyridin-2-yl)oxy)piperidin-1-yl)methanone: Followed General Procedure C using 2-(piperidin-4-yloxy)-4-(trifluoromethyl)pyridine (80 mg, 0.325 mmol, 1 equiv) and 1H-indole-6-carboxylic acid (78.5 mg, 0.488 mmol, 1.5 equiv). The crude product was purified by reverse flash chromatography with the following conditions: column, C18 silica gel; mobile phase, ACN in water, 0% to 100% gradient in 30 min; detector, UV 254 nm. This provided (1H-indol-6-yl)(4-((4-(trifluoromethyl)pyridin-2-yl)oxy)piperidin-1-yl)methanone (20 mg, 50.6%) as a white solid. ¹H NMR (400 MHZ, DMSO-d₆) δ 11.27 (s, 1H), 8.45-8.43 (m, 1H), 8.13-8.10 (m, 1H), 7.59-7.57 (m, 1H), 7.53-7.41 (m, 2H), 7.19-7.16 (m, 1H), 7.07-7.05 (m, 1H), 6.48-6.47 (m, 1H), 5.51-5.48 (m, 1H), 3.69 (s, 4H), 2.08-2.01 (m, 2H), 1.75 (s, 2H). MS m/z: 390.2 [M+H]⁺.

### (1H-Indol-6-yl)(4-((4-(trifluoromethyl)pyridin-2-yl)oxy)piperidin-1-yl)methanone (322)

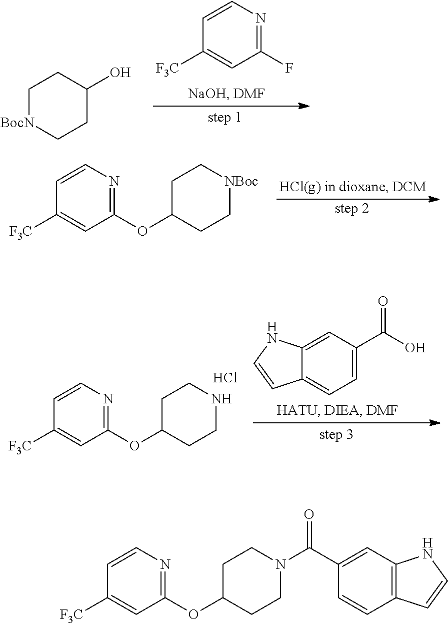

Step 1: tert-butyl 4-((4-(trifluoromethyl)pyridin-2-yl)oxy)piperidine-1-carboxylate: Followed General Procedure D using tert-butyl 4-hydroxypiperidine-1-carboxylate (100 mg. 0.497 mmol, 1 equiv) and 2-fluoro-4-(trifluoromethyl)pyridine (82 mg, 0.497 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 4-((4-(trifluoromethyl)pyridin-2-yl)oxy)piperidine-1-carboxylate (120 mg, 69.73%) as a white solid. MS m/z: 347 [M+H]⁺.

Step 2: 2-(piperidin-4-yloxy)-4-(trifluoromethyl)pyridine hydrochloride: Followed General Procedure B using tert-butyl 4-((4-(trifluoromethyl)pyridin-2-yl)oxy)piperidine-1-carboxylate (120 mg, 0.674 mmol, 1 equiv) to afford the crude product 2-(piperidin-4-yloxy)-4-(trifluoromethyl)pyridine hydrochloride (80 mg). MS m/z: 247 [M+H]⁺.

Step 3: (1H-indol-6-yl)(4-((4-(trifluoromethyl)pyridin-2-yl)oxy)piperidin-1-yl)methanone: Followed General Procedure E using 2-(piperidin-4-yloxy)-4-(trifluoromethyl)pyridine (80 mg, 0.325 mmol, 1 equiv) and 1H-indole-6-carboxylic acid (78.5 mg, 0.488 mmol, 1.5 equiv). The crude product was purified by reverse flash chromatography with the following conditions: column, C18 silica gel; mobile phase, ACN in water, 0% to 100% gradient in 30 min; detector, UV 254 nm. This provided (1H-indol-6-yl)(4-((4-(trifluoromethyl)pyridin-2-yl)oxy)piperidin-1-yl)methanone (20 mg, 50.6%) as a white solid. ¹H NMR (400 MHZ, DMSO-d₆) δ 11.28 (s, 1H), 8.43 (d, J=5.3 Hz, 1H), 7.58 (d, J=8.1 Hz, 1H), 7.49-7.43 (m, 2H), 7.32 (dd, J=5.4, 1.5 Hz, 1H), 7.19 (s, 1H), 7.05 (dd, J=8.1, 1.5 Hz, 1H), 6.51-6.45 (m, 1H), 5.44-5.27 (m, 1H), 4.20-3.60 (m, 2H) 3.46-3.36 (m, 2H), 2.04 (s, 2H), 1.69 (s, 2H). MS m/z: 390.1 [M+H]⁺.

### 3-({1-[1-(2,2-Difluoroethyl)-1H-pyrazolo[3,4-b]pyrazin-6-yl]piperidin-4-yl}methoxy)-2-(trifluoromethyl)pyridine (323)

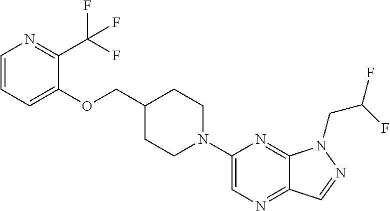 [SMILES: FC(F)Cn1ncc2ncc(N3CCC(COc4cccnc4C(F)(F)F)CC3)nc21]

Step 1: tert-butyl 4-({[2-(trifluoromethyl)pyridin-3-yl]oxy}methyl)piperidine-1-carboxylate: Followed General Procedure D using tert-butyl 4-(hydroxymethyl)piperidine-1-carboxylate (50 mg, 0.23 mmol, 1 equiv) and 3-fluoro-2-(trifluoromethyl)pyridine (38 mg, 0.23 mmol, 1 equiv) to afford tert-butyl 4-({[2-(trifluoromethyl)pyridin-3-yl]oxy}methyl)piperidine-1-carboxylate (55 mg, 66%) as a white solid. MS m/z: 361 [M+H]⁺.

Step 2: 3-[(piperidin-4-yl)methoxy]-2-(trifluoromethyl)pyridine hydrochloride: Followed General Procedure B using tert-butyl 4-({[2-(trifluoromethyl)pyridin-3-yl]oxy}methyl)piperidine-1-carboxylate (55 mg, 0.15 mmol, 1 equiv) to afford the crude product 3-[(piperidin-4-yl)methoxy]-2-(trifluoromethyl)pyridine hydrochloride (40 mg). MS m/z: 261 [M+H]⁺.

Step 3: 3-({1-[1-(2,2-difluoroethyl)-1H-pyrazolo[3,4-b]pyrazin-6-yl]piperidin-4-yl}methoxy)-2-(trifluoromethyl)pyridine: Followed General Procedure C using 3-[(piperidin-4-yl)methoxy]-2-(trifluoromethyl)pyridine hydrochloride (27 mg, 0.1 mmol, 1 equiv) and 6-chloro-1-(2,2-difluoroethyl)-1H-pyrazolo[3,4-b]pyrazine (20 mg, 0.1 mmol, 1 equiv) to afford 3-({1-[1-(2,2-difluoroethyl)-1H-pyrazolo[3,4-b]pyrazin-6-yl]piperidin-4-yl}methoxy)-2-(trifluoromethyl)pyridine (33 mg, 82%) as a white solid. ¹H NMR (500 MHZ, DMSO-d₆) δ 8.48 (s, 1H), 8.25 (dd, J=4.6, 1.1 Hz, 1H), 8.13 (s, 1H), 7.84-7.74 (m, 1H), 7.69 (dd, J=8.6, 4.5 Hz, 1H), 6.43 (tt, J=54.9, 3.8 Hz, 1H), 4.69 (td, J=15.0, 3.8 Hz, 2H), 4.61 (dd, J=13.1. 3.2 Hz, 2H), 4.08 (d, J=6.3 Hz, 2H), 3.06 (td, J=12.9, 2.6 Hz, 2H), 2.23-2.11 (m, 1H), 1.89 (dd, J=13.2, 3.6 Hz, 2H), 1.38 (qd, J=12.5, 4.1 Hz, 2H). MS m/z: 443.3 [M+H]⁺.

### 2-({1-[1-(2,2-Difluoroethyl)-1H-pyrazolo[3,4-b]pyrazin-6-yl]piperidin-4-yl}methoxy)-4-(trifluoromethyl)pyridine (324)

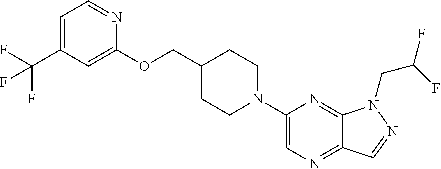 [SMILES: FC(F)Cn1ncc2ncc(N3CCC(COc4cc(C(F)(F)F)ccn4)CC3)nc21]

Step 1: tert-butyl 4-({[4-(trifluoromethyl)pyridin-2-yl]oxy}methyl)piperidine-1-carboxylate: Followed General Procedure D using tert-butyl 4-(hydroxymethyl)piperidine-1-carboxylate (50 mg, 0.23 mmol, 1 equiv) and 2-fluoro-4-(trifluoromethyl)pyridine (38 mg, 0.23 mmol, 1 equiv) to afford tert-butyl 4-({[4-(trifluoromethyl)pyridin-2-yl]oxy}methyl)piperidine-1-carboxylate (47 mg, 56%) as a white solid. MS m/z: 361 [M+H]⁺.

Step 2: 2-[(piperidin-4-yl)methoxy]-4-(trifluoromethyl)pyridine hydrochloride: Followed General Procedure B using tert-butyl 4-({[4-(trifluoromethyl)pyridin-2-yl]oxy}methyl)piperidine-1-carboxylate (47 mg, 0.15 mmol, 1 equiv) to afford the crude product 2-[(piperidin-4-yl)methoxy]-4-(trifluoromethyl)pyridine hydrochloride (40 mg). MS m/z: 261 [M+H]⁺.

Step 3: 2-({1-[1-(2,2-difluoroethyl)-1H-pyrazolo[3,4-b]pyrazin-6-yl]piperidin-4-yl}methoxy)-4-(trifluoromethyl)pyridine: Followed General Procedure C using 2-[(piperidin-4-yl)methoxy]-4-(trifluoromethyl)pyridine hydrochloride (27 mg, 0.1 mmol, 1 equiv) and 6-chloro-1-(2,2-difluoroethyl)-1H-pyrazolo[3,4-b]pyrazine (20 mg, 0.1 mmol, 1 equiv) to afford 2-({1-[1-(2,2-difluoroethyl)-1H-pyrazolo[3,4-b]pyrazin-6-yl]piperidin-4-yl}methoxy)-4-(trifluoromethyl)pyridine (20 mg, 49%) as a white solid. ¹H NMR (500 MHZ, DMSO-d₆) δ 8.50 (s, 1H), 8.45 (d, J=5.3 Hz, 1H), 8.15 (s, 1H), 7.34 (dd, J=5.1, 1.3 Hz, 1H), 7.24 (s, 1H), 6.59-6.33 (m, 1H), 4.71 (td, J=15.0, 3.8 Hz, 2H), 4.63 (d, J=13.2 Hz, 2H), 4.26 (d, J=6.5 Hz, 2H), 3.07 (td, J=12.9, 2.6 Hz, 2H), 2.23-2.12 (m, 1H), 1.97-1.87 (m, 2H), 1.45-1.32 (m, 2H). MS m/z: 443.3 [M+H]⁺.

### 1-(2,2-Difluoroethyl)-6-(4-((2-(trifluoromethyl)pyridin-3-yl)oxy)piperidin-1-yl)-1H-pyrazolo[3,4-b]pyrazine (325)

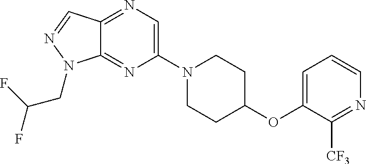 [SMILES: FC(F)Cn1ncc2ncc(N3CCC(Oc4cccnc4C(F)(F)F)CC3)nc21]

Followed General Procedure C using 3-(piperidin-4-yloxy)-2-(trifluoromethyl)pyridine hydrochloride (70 mg, 0.284 mmol, 1.00 equiv) and 6-chloro-1-(2,2-difluoroethyl)-1H-pyrazolo[3,4-b]pyrazine (61.9 mg, 0.284 mmol, 1.00 equiv). The crude product was purified by reverse flash chromatography with the following conditions (column, C18 gel; mobile phase, B phase: MeCN, A phase: water; 5% to 95% B gradient in 20 min; detector: UV 254/220 nm). This provided 1-(2,2-difluoroethyl)-6-(4-((2-(trifluoromethyl)pyridin-3-yl)oxy)piperidin-1-yl)-1H-pyrazolo[3,4-b]pyrazine (60 mg, 49.3%) as a white solid. ¹H NMR (400 MHZ, DMSO-d₆) δ 8.51 (s, 1H), 8.27 (dd, J=4.6, 1.2 Hz, 1H), 8.15 (s, 1H), 7.95 (d, J=8.6 Hz, 1H), 7.71 (dd, J=8.6, 4.5 Hz, 1H), 6.44 (tt, J=54.9, 3.8 Hz, 1H), 5.04-4.97 (m, 1H), 4.70 (td, J=15.0, 3.8 Hz, 2H), 4.01-3.90 (m, 2H), 3.86-3.75 (m, 2H), 2.12-2.02 (m, 2H), 1.84-1.72 (m, 2H). MS m/z: 429.1 [M+H]⁺.

### 1-(2,2-Difluoroethyl)-6-(4-((4-(trifluoromethyl)pyridin-2-yl)oxy)piperidin-1-yl)-1H-pyrazolo[3,4-b]pyrazine (326)

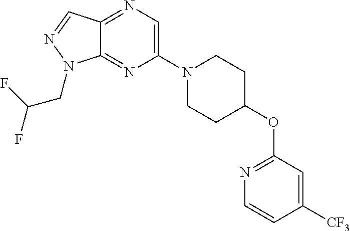 [SMILES: FC(F)Cn1ncc2ncc(N3CCC(Oc4cc(C(F)(F)F)ccn4)CC3)nc21]

Followed General Procedure C using 2-(piperidin-4-yloxy)-4-(trifluoromethyl)pyridine hydrochloride (91.7 mg, 0.325 mmol, 1.00 equiv) and 6-chloro-1-(2,2-difluoroethyl)-1H-pyrazolo[3,4-b]pyrazine (70.8 mg, 0.325 mmol, 1.00 equiv). The crude product was purified by reverse flash chromatography with the following conditions (column, C18 gel; mobile phase, B phase: MeCN, A phase: water; 5% to 95% B gradient in 20 min; detector: UV 254/220 nm). This provided 1-(2,2-difluoroethyl)-6-(4-((4-(trifluoromethyl)pyridin-2-yl)oxy)piperidin-1-yl)-1H-pyrazolo[3,4-b]pyrazine (40 mg, 28.7%) as a white solid. ¹H NMR (400 MHZ, DMSO-d₆) δ8.52 (s, 1H), 8.46 (d, J=5.4, 0.8 Hz, 1H), 8.15 (s, 1H), 7.37-7.31 (m, 1H), 7.23-7.18 (m, 1H), 6.45 (tt, J=54.9, 3.8 Hz, 1H), 5.40 (tt, J=8.1, 3.9 Hz, 1H), 4.71 (td, J=15.0, 3.8 Hz, 2H), 4.25-4.15 (m, 2H), 3.68-3.57 (m, 2H), 2.18-2.06 (m, 2H), 1.83-1.70 (m, 2H). MS m/z: 429.1 [M+H]⁺.

### (1H-Indol-6-yl)(4-((6-(trifluoromethyl)pyridin-2-yl)oxy)piperidin-1-yl)methanone (327)

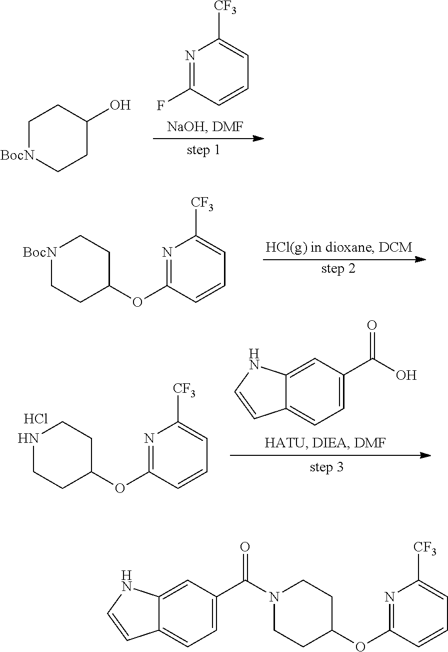

Step 1: tert-butyl 4-((6-(trifluoromethyl)pyridin-2-yl)oxy)piperidine-1-carboxylate: Followed General Procedure D using tert-butyl 4-hydroxypiperidine-1-carboxylate (100 mg, 0.497 mmol, 1 equiv) and 2-fluoro-6-(trifluoromethyl)pyridine (82 mg, 0.497 mmol, 1 equiv). The crude product was purified by reverse flash chromatography with the following conditions: column, C18 silica gel; mobile phase, MeCN in Water (0.1% FA), 0% to 100% gradient in 20 min; detector, UV 254 nm. This provided tert-butyl 4-((6-(trifluoromethyl)pyridin-2-yl)oxy)piperidine-1-carboxylate (120 mg, 69.73%) as a white solid. MS m/z: 347 [M+H]⁺.

Step 2: 2-(piperidin-4-yloxy)-6-(trifluoromethyl)pyridine hydrochloride: Followed General Procedure B using tert-butyl 4-((6-(trifluoromethyl)pyridin-2-yl)oxy)piperidine-1-carboxylate (220 mg, 0.674 mmol, 1 equiv) to afford the crude product 2-(piperidin-4-yloxy)-6-(trifluoromethyl)pyridine hydrochloride (80 mg). MS m/z: 247 [M+H]⁺.

Step 3: (1H-indol-6-yl)(4-((6-(trifluoromethyl)pyridin-2-yl)oxy)piperidin-1-yl)methanone: Followed General Procedure C using 2-(piperidin-4-yloxy)-6-(trifluoromethyl)pyridine (80 mg, 0.325 mmol, 1 equiv) and 1H-indole-6-carboxylic acid (78.5 mg, 0.488 mmol, 1.5 equiv). The crude product was purified by reverse flash chromatography with the following conditions: column, C18 silica gel; mobile phase, ACN in water, 10% to 50% gradient in 10 min; detector, UV 254 nm. This provided (1H-indol-6-yl)(4-((6-(trifluoromethyl)pyridin-2-yl)oxy)piperidin-1-yl)methanone (78 mg, 61.6%) as a white solid. ¹H NMR (400 MHZ, DMSO-d₆) δ 11.39-11.15 (m, 1H), 7.97 (t, J=7.9 Hz, 1H), 7.59-7.56 (m, 1H), 7.49-7.43 (m, 3H), 7.17-7.02 (m, 2H), 6.54-6.39 (m, 1H), 5.43-5.14 (m, 1H), 3.55-3.39 (m, 2H), 2.14-1.93 (m, 2H), 1.83-1.61 (m, 2H). MS m/z: 390.2 [M+H]⁺.

### 2-({1-[1-(2,2-Difluoroethyl)-1H-pyrazolo[3,4-b]pyrazin-6-yl]piperidin-4-yl}methoxy)-6-(trifluoromethyl)pyridine (328)

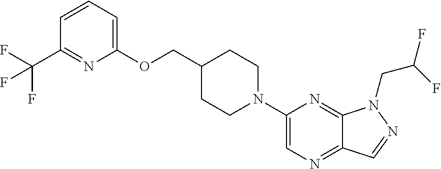 [SMILES: FC(F)Cn1ncc2ncc(N3CCC(COc4cccc(C(F)(F)F)n4)CC3)nc21]

Step 1: tert-butyl 4-({[6-(trifluoromethyl)pyridin-2-yl]oxy}methyl)piperidine-1-carboxylate: Followed General Procedure D using tert-butyl 4-(hydroxymethyl)piperidine-1-carboxylate (50 mg, 0.23 mmol, 1 equiv) and 2-bromo-6-(trifluoromethyl)pyridine (53 mg, 0.23 mmol, 1 equiv) to afford tert-butyl 4-({[6-(trifluoromethyl)pyridin-2-yl]oxy}methyl)piperidine-1-carboxylate (54 mg, 65%) as a white solid. MS m/z: 361 [M+H]⁺.

Step 2: 2-[(piperidin-4-yl)methoxy]-6-(trifluoromethyl)pyridine hydrochloride: Followed General Procedure B using tert-butyl 4-({[6-(trifluoromethyl)pyridin-2-yl]oxy}methyl)piperidine-1-carboxylate (54 mg, 0.15 mmol, 1 equiv) to afford the crude product 2-[(piperidin-4-yl)methoxy]-6-(trifluoromethyl)pyridine hydrochloride (40 mg). MS m/z: 261 [M+H]⁺.

Step 3: 2-({1-[1-(2,2-difluoroethyl)-1H-pyrazolo[3,4-b]pyrazin-6-yl]piperidin-4-yl}methoxy)-6-(trifluoromethyl)pyridine: Followed General Procedure C using 2-[(piperidin-4-yl)methoxy]-6-(trifluoromethyl)pyridine hydrochloride (27 mg, 0.1 mmol, 1 equiv) and 6-chloro-1-(2,2-difluoroethyl)-1H-pyrazolo[3,4-b]pyrazine (20 mg, 0.1 mmol, 1 equiv) to afford 2-({1-[1-(2,2-difluoroethyl)-1H-pyrazolo[3,4-b]pyrazin-6-yl]piperidin-4-yl}methoxy)-6-(trifluoromethyl)pyridine (28 mg, 70%) as a white solid. ¹H NMR (500 MHZ, DMSO-d₆) δ 8.47 (s, 1H), 8.12 (s, 1H), 7.96 (ddd, J=8.4, 7.4, 0.8 Hz, 1H), 7.47 (d, J=7.3 Hz, 1H), 7.16 (d, J=8.4 Hz, 1H), 6.43 (tt, J=54.9, 3.8 Hz, 1H), 4.69 (td, J=15.0, 3.8 Hz, 2H), 4.61 (d, J=13.1 Hz, 2H), 4.19 (d, J=6.4 Hz, 2H), 3.04 (td, J=13.0, 2.6 Hz, 2H), 2.19-2.07 (m, 1H), 1.94-1.84 (m, 2H), 1.36 (qd, J=12.4, 4.1 Hz, 2H). MS m/z: 443.3 [M+H]⁺.

### 1-(2,2-Difluoroethyl)-6-(4-((1-(2-(trifluoromethyl)pyridin-3-yl)azetidin-3-yl)oxy)piperidin-1-yl)-1H-pyrazolo[3,4-b]pyrazine (329)

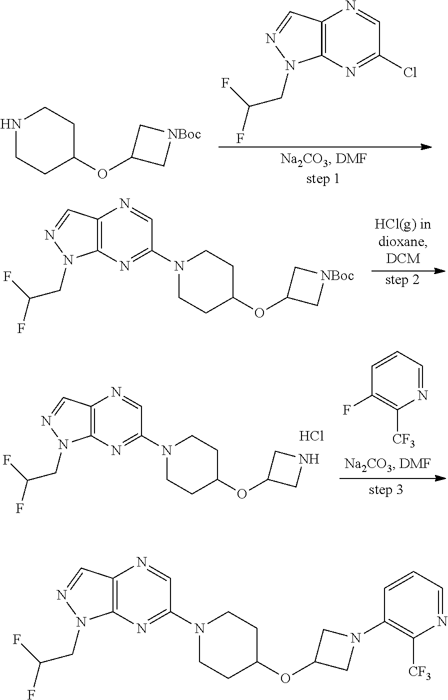

Step 1: tert-butyl 3-((1-(1-(2,2-difluoroethyl)-1H-pyrazolo[3,4-b]pyrazin-6-yl)piperidin-4-yl)oxy)azetidine-1-carboxylate: Followed General Procedure C using 6-chloro-1-(2,2-difluoroethyl)pyrazolo[3,4-b]pyrazine (100 mg, 0.457 mmol, 1 equiv) and tert-butyl 3-(piperidin-4-yloxy)azetidine-1-carboxylate (129.00 mg, 0.503 mmol, 1.1 equiv) to afford tert-butyl 1-oxo-2-(6-(trifluoromethyl)pyridin-2-yl)-2,8-diazaspiro[4.5]decane-8-carboxylate (220 mg, 46%) as a colorless oil. MS m/z: 400 [M+H]⁺.

Step 2: 6-(4-(azetidin-3-yloxy)piperidin-1-yl)-1-(2,2-difluoroethyl)-1H-pyrazolo[3,4-b]pyrazine hydrochloride: Followed General Procedure B using tert-butyl 3-((1-(1-(2,2-difluoroethyl)-1H-pyrazolo[3,4-b]pyrazin-6-yl)piperidin-4-yl)oxy)azetidine-1-carboxylate (60 mg, 0.137 mmol, 1 equiv) to afford the crude product 3-((o-tolyloxy)methyl)pyrrolidine (100 mg) was directly used in next step without further purification. MS m/z: 339 [M+H]⁺.

Step 3: 1-(2,2-difluoroethyl)-6-(4-((1-(2-(trifluoromethyl)pyridin-3-yl)azetidin-3-yl)oxy)piperidin-1-yl)-1H-pyrazolo[3,4-b]pyrazine: Followed General Procedure C using 6-(4-(azetidin-3-yloxy)piperidin-1-yl)-1-(2,2-difluoroethyl)-1H-pyrazolo[3,4-b]pyrazine hydrochloride (50 mg, 0.148 mmol, 1 equiv) and 3-fluoro-2-(trifluoromethyl)pyridine (26.8 mg. 0.163 mmol, 1.1 equiv). The crude product was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 1-(2,2-difluoroethyl)-6-(4-((1-(2-(trifluoromethyl)pyridin-3-yl)azetidin-3-yl)oxy)piperidin-1-yl)-1H-pyrazolo[3,4-b]pyrazine (20.2 mg, 28.2%) as a white solid. ¹H NMR (400 MHZ, DMSO-d₆) δ 8.48 (s, 1H), 8.13 (s, 1H), 8.01 (d, J=4.2 Hz, 1H), 7.47 (t. J=5.7 Hz, 1H), 7.12 (d, J=8.6 Hz, 1H), 6.44 (t, J=55.1 Hz, 1H), 4.70 (t, J=15.1 Hz, 2H), 4.60 (s, 1H), 4.33-4.20 (m, 4H), 3.79 (d, J=45.8 Hz, 4H), 1.95 (s, 2H), 1.52 (d, J=11.3 Hz, 2H). MS m/z: 484.1 [M+H]⁺.

### 2-(6-(4-((5-(Trifluoromethyl)pyridin-2-yl)oxy)piperidin-1-yl)pyrazin-2-yl)-1,3,4-thiadiazole (330)

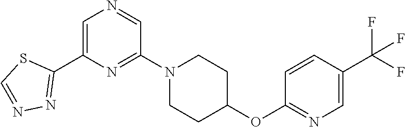 [SMILES: FC(F)(F)c1ccc(OC2CCN(c3cncc(-c4nncs4)n3)CC2)nc1]

Followed General Procedure C using 2-(piperidin-4-yloxy)-5-(trifluoromethyl)pyridine hydrochloride (81.8 mg, 0.332 mmol, 1.10 equiv) and 2-(6-chloropyrazin-2-yl)-1,3,4-thiadiazole (60 mg, 0.302 mmol, 1.00 equiv). The crude product was purified by reverse flash chromatography with the following conditions (column, C18 gel; mobile phase, B phase: MeCN, A phase: water; 0% to 100% B gradient in 20 min; detector: UV 254/220 nm). This provided 2-(6-(4-((5-(trifluoromethyl)pyridin-2-yl)oxy)piperidin-1-yl)pyrazin-2-yl)-1,3,4-thiadiazole (84 mg, 68.1%) as a yellow green solid. ¹HNMR (400 MHZ, DMSO-d₆) δ 9.72 (s, 1H), 8.65-8.57 (m, 3H). 8.10-8.06 (m, 1H), 7.03 (d, J=8.7 Hz, 1H), 5.44-5.37 (m, 1H), 4.15-4.08 (m, 2H), 3.59-3.51 (m, 2H), 2.18-2.10 (m, 2H), 1.83-1.73 (m, 2H). MS m/z: 409.1 [M+H]⁺.

### 2-((1-(6-(1,3,4-Thiadiazol-2-yl)pyrazin-2-yl)piperidin-4-yl)oxy)nicotinonitrile (331)

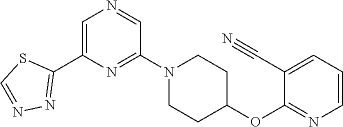 [SMILES: N#Cc1cccnc1OC1CCN(c2cncc(-c3nncs3)n2)CC1]

Followed General Procedure C using 2-(6-chloropyrazin-2-yl)-1,3,4-thiadiazole (100 mg, 0.503 mmol, 1 equiv) and 2-(piperidin-4-yloxy)nicotinonitrile hydrochloride (112 mg, 0.553 mmol, 1.1 equiv). The crude product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 2-((1-(6-(1,3,4-thiadiazol-2-yl)pyrazin-2-yl)piperidin-4-yl)oxy)nicotinonitrile (50 mg, 27.18%) as an white solid. ¹H NMR (400 MHZ, DMSO-d₆) δ 9.72 (s, 1H), 8.65 (s, 1H), 8.59 (s, 1H), 8.49 (dd, J=5.0, 2.0 Hz, 1H), 8.29 (dd, J=7.6, 2.0 Hz, 1H), 7.20 (dd, J=7.6, 5.0 Hz, 1H), 5.50-5.43 (m, 1H), 4.07-3.98 (m, 2H), 3.65 (ddd, J=13.2, 8.1, 3.5 Hz, 2H), 2.12 (dd, J=19.7, 10.1 Hz, 2H), 1.83 (dd, J=12.3, 4.1 Hz, 2H). MS m/z: 366.1 [M+H]⁺.

### 2-((1-(1H-Indole-6-carbonyl)piperidin-4-yl)oxy)-5-(trifluoromethyl)nicotinonitrile (332)

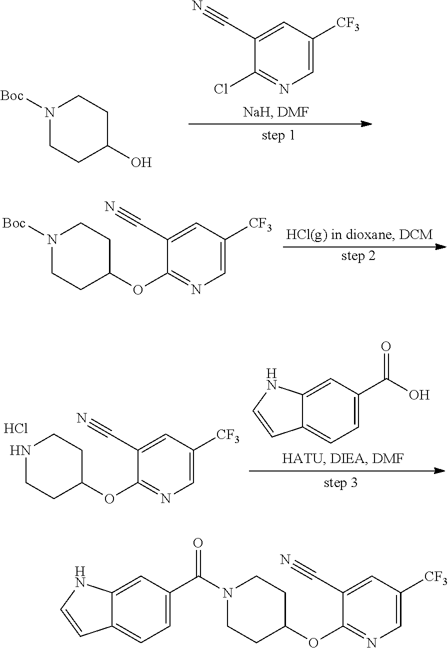

Step 1: tert-butyl 4-((3-cyano-5-(trifluoromethyl)pyridin-2-yl)oxy)piperidine-1-carboxylate: Followed General Procedure D using tert-butyl 4-hydroxypiperidine-1-carboxylate (200 mg, 0.994 mmol, 1 equiv) and 2-chloro-5-(trifluoromethyl)nicotinonitrile (246 mg, 1.19 mmol, 1.2 equiv). The crude product was purified by silica gel column chromatography, eluted with PE/EA (1:1) to afford tert-butyl 4-((3-cyano-5-(trifluoromethyl)pyridin-2-yl)oxy)piperidine-1-carboxylate (120 mg, 32.5%) as a colorless oil. MS m/z: 372 [M+H]⁺.

Step 2: 2-(piperidin-4-yloxy)-5-(trifluoromethyl)nicotinonitrile hydrochloride: Followed General Procedure using tert-butyl 4-((3-cyano-5-(trifluoromethyl)pyridin-2-yl)oxy)piperidine-1-carboxylate (120 mg, 0.323 mmol, 1 equiv) to afford the crude product 2-(piperidin-4-yloxy)-5-(trifluoromethyl)nicotinonitrile hydrochloride (100 mg). MS m/z: 272 [M+H]⁺.

Step 3: 2-((1-(1H-indole-6-carbonyl)piperidin-4-yl)oxy)-5-(trifluoromethyl)nicotinonitrile: Followed General Procedure E using 2-(piperidin-4-yloxy)-5-(trifluoromethyl)nicotinonitrile hydrochloride (100 mg, 0.325 mmol, 1 equiv) and 1H-indole-6-carboxylic acid (52.4 mg, 0.325 mmol, 1 equiv). The crude product was purified by reverse flash chromatography with the following conditions: column, C18 silica gel; mobile phase, MeCN in Water (0.1% FA), 10% to 50% gradient in 20 min; detector, UV 254 nm to afford 2-((1-(1H-indole-6-carbonyl)piperidin-4-yl)oxy)-5-(trifluoromethyl)nicotinonitrile (90 mg, 66.8%) as a white solid. ¹H NMR (400 MHZ, DMSO-d₆) δ 11.28 (d, J=2.6 Hz, 1H), 8.87 (dd, J=18.8, 2.4 Hz, 2H), 7.59 (dd, J=8.2, 1.8 Hz, 1H), 7.52-7.43 (m, 2H), 7.11-7.04 (m, 1H), 6.55-6.45 (m, 1H), 5.56-5.48 (m, 1H), 3.86-3.76 (m, 2H), 3.56-3.47 (m, 2H), 2.12-2.03 (m, 2H), 1.86-1.74 (m, 2H). MS m/z: 415.1 [M+H]⁺.

### (1H-Indol-6-yl)(4-((5-(trifluoromethyl)pyridin-2-yl)oxy)piperidin-1-yl)methanone (333)

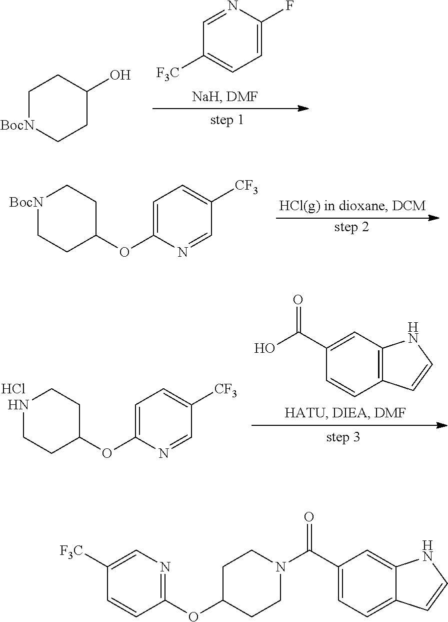

Step 1: tert-butyl 4-((5-(trifluoromethyl)pyridin-2-yl)oxy)piperidine-1-carboxylate: Followed General Procedure D using tert-butyl 4-hydroxypiperidine-1-carboxylate (500 mg, 2.48 mmol, 1 equiv) and 2-fluoro-5-(trifluoromethyl)pyridine (492 mg, 2.98 mmol, 1.2 equiv). The crude product was purified by reverse flash chromatography with the following conditions: column, C18 silica gel; mobile phase, MeCN in water, 0% to 100% gradient in 20 min; detector, UV 254 nm. This provided tert-butyl 4-((5-(trifluoromethyl)pyridin-2-yl)oxy)piperidine-1-carboxylate (710 mg, 78.4%) as a white oil. MS m/z: 347 [M+H]⁺.

Step 2: 2-(piperidin-4-yloxy)-5-(trifluoromethyl)pyridine hydrochloride: Followed General Procedure B using tert-butyl 4-((5-(trifluoromethyl)pyridin-2-yl)oxy)piperidine-1-carboxylate (700 mg, 2.02 mmol, 1 equiv) to afford the crude product 2-(piperidin-4-yloxy)-5-(trifluoromethyl)pyridine hydrochloride (400 mg, 80.38%) as a white solid. MS m/z: 247 [M+H]⁺.

Step 3: (1H-indol-6-yl)(4-((5-(trifluoromethyl)pyridin-2-yl)oxy)piperidin-1-yl)methanone: Followed General Procedure E using 1H-indole-6-carboxylic acid (50 mg, 0.31 mmol, 1 equiv) and 2-(piperidin-4-yloxy)-5-(trifluoromethyl)pyridine hydrochloride (84 mg, 0.34 mmol, 1.1 equiv) The crude product was purified by reverse flash chromatography with the following conditions: column, C18 silica gel; mobile phase, MeCN in water, 10% to 100% gradient in 30 min; detector, UV 254 nm. This provided (1H-indol-6-yl)(4-((5-(trifluoromethyl)pyridin-2-yl)oxy)piperidin-1-yl)methanone (74.4 mg, 61%) as a white solid. ¹H NMR (300 MHz, DMSO-d₆) δ 11.28 (s, 1H), 8.63-8.57 (m, 1H), 8.08 (dd, J=8.8, 2.7 Hz, 1H), 7.58 (d, J=8.1 Hz, 1H), 7.50-7.43 (m, 2H), 7.10-6.98 (m, 2H), 6.50-6.47 (m, 1H), 5.45-5.32 (m, 1H), 4.04-3.65 (m, 2H), 3.51-3.37 (m, 2H) 2.05 (s, 2H), 1.71 (s, 2H). MS m/z: 390.1 [M+H]⁺.

### 2-((1-(1H-Indole-6-carbonyl)piperidin-4-yl)oxy)nicotinonitrile (334)

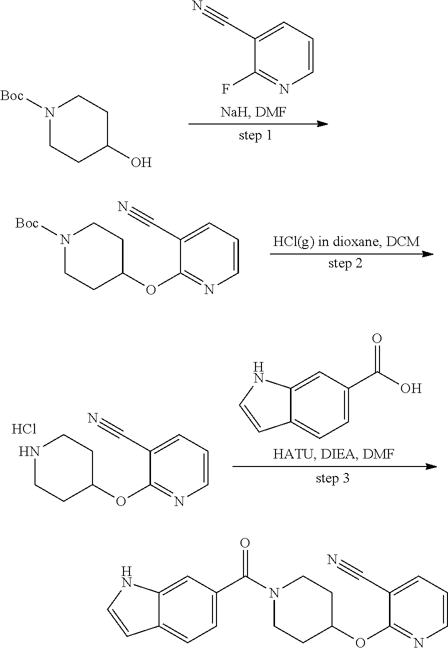

Step 1: tert-butyl 4-((3-cyanopyridin-2-yl)oxy)piperidine-1-carboxylate: Followed General Procedure D using tert-butyl 4-hydroxypiperidine-1-carboxylate (989 mg, 4.91 mmol, 1.2 equiv) and methyl 2-fluoropyridine-3-carbonitrile (500 mg, 4.09 mmol, 1 equiv) to afford tert-butyl 4-((3-cyanopyridin-2-yl)oxy)piperidine-1-carboxylate (240 mg, 19.3%) as a colorless oil. LCMS (ES, m/z): 304 [M+H]⁺.

Step 2: 2-(piperidin-4-yloxy)nicotinonitrile hydrochloride: Followed General Procedure B using tert-butyl 4-((3-cyanopyridin-2-yl)oxy)piperidine-1-carboxylate (240 mg, 0.791 mmol, 1 equiv) to afford the crude product 2-(piperidin-4-yloxy)nicotinonitrile hydrochloride (100 mg) was used directly in next step without further purification. MS m/z: 204 [M+H]⁺.

Step 3: 2-((1-(1H-indole-6-carbonyl)piperidin-4-yl)oxy)nicotinonitrile: Followed General Procedure E using 1H-indole-6-carboxylic acid (100 mg, 0.621 mmol, 1 equiv) and 2-(piperidin-4-yloxy)nicotinonitrile hydrochloride (151 mg, 0.745 mmol, 1.2 equiv). The crude product was purified by silica gel column chromatography, eluted with EtOAc/PE (1:1) to afford the product. The product was further purified by reversed phase Combi-flash chromatography with the following conditions (column, C18 gel; mobile phase, B phase: MeCN, A phase: water; 35% to 75% B gradient in 20 min; detector: UV 254/220 nm). The pure fraction was concentrated under vacuum to afford 2-((1-(1H-indole-6-carbonyl)piperidin-4-yl)oxy)nicotinonitrile (40 mg, 18.6%) as a white solid. ¹H NMR (300 MHz, DMSO-d₆) δ 11.28 (s, 1H), 8.47 (dd, J=5.0, 2.0 Hz, 1H), 8.29 (dd, J=7.6, 1.9 Hz, 1H), 7.59 (d, J=8.1 Hz, 1H), 7.53-7.43 (m, 2H), 7.19 (dd, J=7.6, 5.0 Hz, 1H), 7.07 (dd, J=8.1, 1.4 Hz, 1H), 6.48 (ddd, J=3.0, 1.9, 0.9 Hz, 1H), 5.44 (dt, J=7.9, 4.0 Hz, 1H), 3.82 (s, 2H), 3.48 (t, J=10.2 Hz, 2H), 2.06 (d, J=10.5 Hz, 2H), 1.75 (d, J=8.8 Hz, 2H). MS m/z: 347.1 [M+H]⁺.

### 1-{4-[(Benzyloxy)methyl]piperidin-1-yl}-2-(2-phenyl-1,3-thiazol-4-yl)ethan-1-one (335)

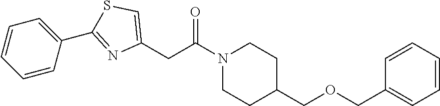 [SMILES: O=C(Cc1csc(-c2ccccc2)n1)N1CCC(COCc2ccccc2)CC1]

Followed General Procedure E using 4-[(benzyloxy)methyl]piperidine (23 mg, 0.11 mmol, 1.0 equiv) and 2-(2-phenyl-1,3-thiazol-4-yl)acetic acid (25 mg, 0.11 mmol) to afford 1-{4-[(benzyloxy)methyl]piperidin-1-yl}-2-(2-phenyl-1,3-thiazol-4-yl)ethan-1-one (38 mg, 82%) as a light yellow oil. ¹H NMR (500 MHZ, CDCl₃) δ 7.95-7.89 (m, 2H), 7.45-7.40 (m, 3H), 7.35-7.27 (m, 5H), 7.15 (s, 1H), 4.72-4.60 (m, 1H), 4.48 (s, 2H), 4.18 (dt, J=13.5, 2.2 Hz, 1H), 4.02-3.87 (m, 2H), 3.29 (qd, J=9.0, 6.3 Hz, 2H), 3.11-3.00 (m, 1H), 2.61 (td, J=12.9, 2.9 Hz, 1H), 1.87 (ddd, J=10.8, 7.4, 4.6 Hz, 1H), 1.80 (tdd, J=13.9, 4.4, 2.3 Hz, 2H), 1.24-1.05 (m, 2H). MS m/z: 407.1 [M+H]⁺.

### 2-(2-Phenyl-1,3-thiazol-4-yl)-1-[4-(2-phenylethoxy)piperidin-1-yl]ethan-1-one (336)

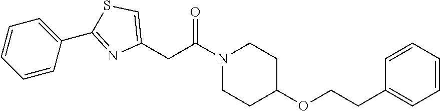 [SMILES: O=C(Cc1csc(-c2ccccc2)n1)N1CCC(OCCc2ccccc2)CC1]

Followed General Procedure E using 4-(2-phenylethoxy)piperidine (23 mg, 0.11 mmol, 1.0 equiv) and 2-(2-phenyl-1,3-thiazol-4-yl)acetic acid (25 mg, 0.11 mmol) to afford 2-(2-phenyl-1,3-thiazol-4-yl)-1-[4-(2-phenylethoxy)piperidin-1-yl]ethan-1-one (37 mg, 80%) as a light yellow oil. ¹H NMR (500 MHZ, CDCl₃) δ 7.94-7.87 (m, 2H), 7.47-7.39 (m, 3H), 7.30-7.27 (m, 1H), 7.23-7.17 (m, 3H), 7.15 (d, J=0.8 Hz, 1H), 3.95 (dd, J=1.6, 0.9 Hz, 2H), 3.85-3.73 (m, 2H), 3.65 (tdd, J=9.2, 7.2, 2.1 Hz, 2H), 3.51 (tt, J=7.2, 3.4 Hz, 1H), 3.42 (dtd, J=13.2, 7.9, 3.7 Hz, 2H), 2.87 (t, J=7.1 Hz, 2H), 1.82-1.68 (m, 2H), 1.55 (ddq, J=20.5, 12.6, 4.4, 4.0 Hz, 4H). MS m/z: 407.1 [M+H]⁺.

### 1-[4-(3-Methylbutoxy)piperidin-1-yl]-2-(2-phenyl-1,3-thiazol-4-yl)ethan-1-one (337)

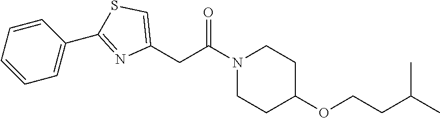 [SMILES: CC(C)CCOC1CCN(C(=O)Cc2csc(-c3ccccc3)n2)CC1]

Followed General Procedure E using 4-(3-methylbutoxy)piperidine (20 mg, 0.11 mmol, 1.0 equiv) and 2-(2-phenyl-1,3-thiazol-4-yl)acetic acid (25 mg, 0.11 mmol) to afford 1-[4-(3-methylbutoxy)piperidin-1-yl]-2-(2-phenyl-1,3-thiazol-4-yl)ethan-1-one (34 mg, 80%) as an off-white solid. ¹H NMR (500 MHz, CDCl₃) δ 7.94-7.88 (m, 2H), 7.42 (td, J=4.6, 4.2, 2.6 Hz, 3H), 7.16 (s, 1H), 3.96 (s, 2H), 3.95-3.84 (m, 2H), 3.52-3.42 (m, 4H), 3.42-3.34 (m, 1H), 1.80 (dt, J=9.7, 4.7 Hz, 2H), 1.74-1.64 (m, 1H), 1.54 (dt, J=8.5, 4.3 Hz, 2H), 1.45 (q, J=6.8 Hz, 2H), 0.90 (s, 3H), 0.88 (s, 3H). MS m/z: 373.5 [M+H]⁺.

### 1-[4-(Pentyloxy)piperidin-1-yl]-2-(2-phenyl-1,3-thiazol-4-yl)ethan-1-one (338)

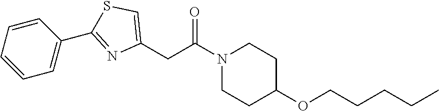 [SMILES: CCCCCOC1CCN(C(=O)Cc2csc(-c3ccccc3)n2)CC1]

Followed General Procedure E using 4-(pentyloxy)piperidine hydrochloride (24 mg, 0.11 mmol, 1.0 equiv) and 2-(2-phenyl-1,3-thiazol-4-yl)acetic acid (25 mg, 0.11 mmol) to afford 1-[4-(pentyloxy)piperidin-1-yl]-2-(2-phenyl-1,3-thiazol-4-yl)ethan-1-one (34 mg, 80%) as a light-yellow solid. ¹H NMR (500 MHZ, CDCl₃) δ 7.95-7.84 (m, 2H), 7.46-7.37 (m, 3H), 7.16 (s, 1H), 3.97 (s, 2H), 3.96-3.84 (m, 2H), 3.53-3.31 (m, 5H), 1.87-1.74 (m, 2H), 1.60-1.51 (m, 4H), 1.34-1.25 (m, 4H), 0.94-0.86 (m, 3H). MS m/z: 373.5 [M+H]⁺.

### 1-(4-Butoxypiperidin-1-yl)-2-(2-phenyl-1,3-thiazol-4-yl)ethan-1-one (339)

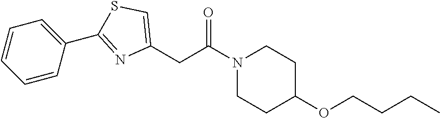 [SMILES: CCCCOC1CCN(C(=O)Cc2csc(-c3ccccc3)n2)CC1]

Followed General Procedure E using 4-butoxypiperidine (18 mg, 0.11 mmol, 1.0 equiv) and 2-(2-phenyl-1,3-thiazol-4-yl)acetic acid (25 mg, 0.11 mmol) to afford 1-(4-butoxypiperidin-1-yl)-2-(2-phenyl-1,3-thiazol-4-yl)ethan-1-one (28 mg, 69%) as an off-white solid. ¹H NMR (500 MHZ, CDCl₃) δ 7.95-7.87 (m, 2H), 7.42 (dt, J=5.0, 3.1 Hz, 3H), 7.16 (s, 1H), 3.96 (s, 2H), 3.95-3.85 (m, 2H), 3.51-3.35 (m, 5H), 1.87-1.74 (m, 2H), 1.54 (qd, J=8.2, 7.6, 5.0 Hz, 4H), 1.42-1.31 (m, 2H), 0.91 (t, J=7.4 Hz, 3H). MS m/z: 359.5 [M+H]⁺.

### (1H-indol-6-yl)(3-(phenoxymethyl)-8-azabicyclo[3.2.1]octan-8-yl)methanone (340)

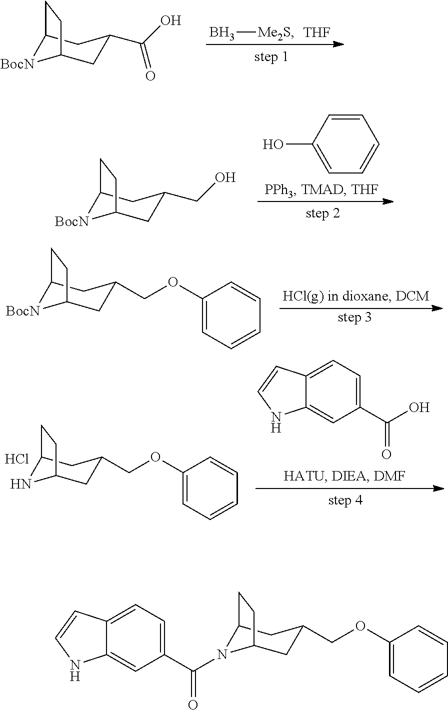

Step 1: tert-butyl 3-(hydroxymethyl)-8-azabicyclo[3.2.1]octane-8-carboxylate: To a stirred solution of 8-(tert-butoxycarbonyl)-8-azabicyclo[3.2.1]octane-3-carboxylic acid (200 mg, 0.783 mmol, 1.00 equiv) in THF (3 mL) was added BH₃-Me₂S (119 mg, 1.56 mmol, 2 equiv) dropwise at 0° C. under N₂ atmosphere. The resulting mixture was warmed to room temperature and stirred overnight under N₂ atmosphere. Desired product could be detected by LCMS. The reaction was quenched with water at 0° C. The resulting mixture was extracted with EtOAc (3×20 mL). The combined organic layers were washed with brine (1×50 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 5% to 60% gradient in 20 min; detector, UV 254 nm. This provided tert-butyl 3-(hydroxymethyl)-8-azabicyclo[3.2.1]octane-8-carboxylate (170 mg, 89.9%) as a colorless oil. MS m/z: 186 [M+H-tBu]⁺.

Step 2: tert-butyl 3-(phenoxymethyl)-8-azabicyclo[3.2.1]octane-8-carboxylate: To a stirred mixture of tert-butyl 3-(hydroxymethyl)-8-azabicyclo[3.2.1]octane-8-carboxylate (170 mg, 0.704 mmol, 1.00 equiv), phenol (133 mg, 1.41 mmol, 2 equiv) and PPh₃ (277 mg, 1.05 mmol, 1.5 equiv) in THF (3 mL) was added TMAD (182 mg, 1.06 mmol, 1.5 equiv) in portion at 0° C. The resulting mixture was warmed to room temperature and stirred for overnight at room temperature. Desired product could be detected by LCMS. The resulting mixture was concentrated under reduced pressure. The residue was purified by silica gel column chromatography, eluted with EtOAc/PE (1/5) to afford tert-butyl 3-(phenoxymethyl)-8-azabicyclo[3.2.1]octane-8-carboxylate (180 mg, 80.5%) as a colorless semi-solid. MS m/z: 262 [M+H-tBu]⁺.

Step 3: 3-(phenoxymethyl)-8-azabicyclo[3.2.1]octane hydrochloride: Followed General Procedure B using tert-butyl 3-(phenoxymethyl)-8-azabicyclo[3.2.1]octane-8-carboxylate (180 mg, 0.567 mmol, 1.00 equiv) to afford the crude product 3-(phenoxymethyl)-8-azabicyclo[3.2.1]octane hydrochloride (140 mg). MS m/z: 218 [M+H]⁺.

Step 4: (1H-indol-6-yl)(3-(phenoxymethyl)-8-azabicyclo[3.2.1]octan-8-yl)methanone: Followed General Procedure E using 1H-indole-6-carboxylic acid (44.5 mg, 0.276 mmol, 1 equiv) and 3-(phenoxymethyl)-8-azabicyclo[3.2.1]octane hydrochloride (70 mg, 0.276 mmol, 1.00 equiv). The crude product was purified by Prep-TLC (EtOAc/PE=1/1) to afford crude product. The crude product was purified by reverse flash chromatography with the following conditions: column, C18 silica gel; mobile phase, MeCN in water, 35% to 70% gradient in 15 min; detector, UV 254 nm. This provided (1H-indol-6-yl)(3-(phenoxymethyl)-8-azabicyclo[3.2.1]octan-8-yl)methanone (30 mg, 30.1%) as a white solid. ¹H NMR (400 MHZ, DMSO-d₆) δ 11.26 (s, 1H), 7.63-7.51 (m, 2H), 7.46 (t, 1H), 7.33-7.22 (m, 2H), 7.13 (dd, 1H), 7.00-6.83 (m, 3H), 6.47 (t, 1H), 4.68 (s, 1H), 4.19 (s, 1H), 3.82 (d, 2H), 2.45-2.38 (m, 1H), 2.00-1.84 (m, 2H), 1.84-1.65 (m, 4H), 1.56 (s, 2H). MS m/z: 361.2 [M+H]⁺.

### 2-(2-Phenyl-1,3-thiazol-4-yl)-1-(4-{[2-(trifluoromethyl)pyridin-4-yl]oxy}piperidin-1-yl)ethan-1-one (399)

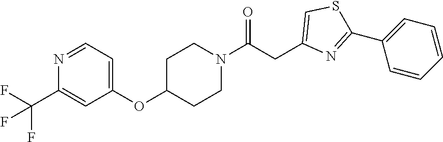 [SMILES: O=C(Cc1csc(-c2ccccc2)n1)N1CCC(Oc2ccnc(C(F)(F)F)c2)CC1]

Followed General Procedure E using 4-(piperidin-4-yloxy)-2-(trifluoromethyl)pyridine hydrochloride (32 mg, 0.11 mmol, 1.0 equiv) and 2-(2-phenyl-1,3-thiazol-4-yl)acetic acid (25 mg, 0.11 mmol, 1.0 equiv) to afford 2-(2-phenyl-1,3-thiazol-4-yl)-1-(4-{[2-(trifluoromethyl)pyridin-4-yl]oxy}piperidin-1-yl)ethan-1-one (28 mg, 69%) as a light yellow oil. ¹H NMR (500 MHZ, CDCl₃) δ 8.52 (d, J=5.7 Hz, 1H), 7.92-7.85 (m, 2H), 7.43-7.37 (m, 3H), 7.18 (d, J=0.9 Hz, 1H), 7.15 (d, J=2.4 Hz, 1H), 6.92 (dd, J=5.7, 2.4 Hz, 1H), 4.68 (tt, J=6.6, 3.4 Hz, 1H), 4.03-3.93 (m, 2H), 3.91-3.82 (m, 1H), 3.81-3.69 (m, 3H), 1.98-1.89 (m, 2H), 1.87-1.77 (m, 2H). MS m/z: 448.5 [M+H]⁺.

### 2-(2-Phenyl-1,3-thiazol-4-yl)-1-(4-{[2-(trifluoromethyl)pyridin-3-yl]oxy}piperidin-1-yl)ethan-1-one (400)

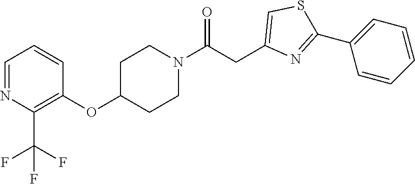 [SMILES: O=C(Cc1csc(-c2ccccc2)n1)N1CCC(Oc2cccnc2C(F)(F)F)CC1]

Followed General Procedure E using 3-(piperidin-4-yloxy)-2-(trifluoromethyl)pyridine hydrochloride (32 mg, 0.11 mmol, 1.0 equiv) and 2-(2-phenyl-1,3-thiazol-4-yl)acetic acid (25 mg, 0.11 mmol, 1.0 equiv) to afford 2-(2-phenyl-1,3-thiazol-4-yl)-1-(4-{[2-(trifluoromethyl)pyridin-3-yl]oxy}piperidin-1-yl)ethan-1-one (46 mg, 90%) as a light yellow oil. ¹H NMR (500 MHZ, CDCl₃) δ 8.25 (dd, J=4.5, 1.2 Hz, 1H), 7.92-7.86 (m, 2H), 7.45-7.37 (m, 4H), 7.32 (d, J=8.6 Hz, 1H), 7.17 (s, 1H), 4.71 (tt, J=5.2, 3.2 Hz, 1H), 4.08-3.84 (m, 4H), 3.76 (ddd, J=13.6, 10.0, 3.3 Hz, 1H), 3.47 (ddd, J=13.6, 9.9, 3.8 Hz, 1H), 1.95-1.80 (m, 4H). MS m/z: 448.5 [M+H]⁺.

### 2-(2-Phenyl-1,3-thiazol-4-yl)-1-(4-{[3-(trifluoromethyl)pyridin-2-yl]oxy}piperidin-1-yl)ethan-1-one (401)

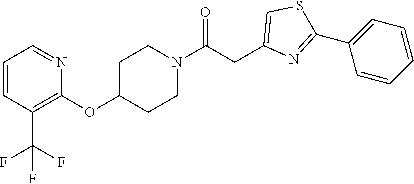 [SMILES: O=C(Cc1csc(-c2ccccc2)n1)N1CCC(Oc2ncccc2C(F)(F)F)CC1]

Followed General Procedure E using 2-(piperidin-4-yloxy)-3-(trifluoromethyl)pyridine hydrochloride (32 mg, 0.11 mmol, 1.0 equiv) and 2-(2-phenyl-1,3-thiazol-4-yl)acetic acid (25 mg, 0.11 mmol, 1.0 equiv) to afford 2-(2-phenyl-1,3-thiazol-4-yl)-1-(4-{[3-(trifluoromethyl)pyridin-2-yl]oxy}piperidin-1-yl)ethan-1-one (12 mg, 24%) as a colorless oil. ¹H NMR (500 MHZ, CDCl₃) δ 8.26 (ddd, J=5.0, 1.9, 0.7 Hz, 1H), 7.94-7.87 (m, 2H), 7.84 (ddd, J=7.6, 2.0, 0.8 Hz, 1H), 7.44-7.36 (m, 3H), 7.17 (d, J=0.9 Hz, 1H), 6.94 (ddd, J=7.5, 5.0, 0.8 Hz, 1H), 5.50-5.44 (m, 1H), 4.03-3.90 (m, 3H), 3.78 (dtt, J=22.7, 13.5, 4.4 Hz, 2H), 3.55 (ddd, J=13.4, 8.1, 5.2 Hz, 1H), 1.89 (dq, J=14.1, 4.3 Hz, 4H). MS m/z: 448.5 [M+H]⁺.

### 2-(2-Phenyl-1,3-thiazol-4-yl)-1-(4-{[6-(trifluoromethyl)pyridin-2-yl]oxy}piperidin-1-yl)ethan-1-one (402)

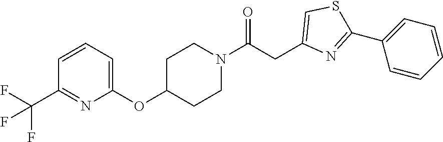 [SMILES: O=C(Cc1csc(-c2ccccc2)n1)N1CCC(Oc2cccc(C(F)(F)F)n2)CC1]

Followed General Procedure E using 2-(piperidin-4-yloxy)-6-(trifluoromethyl)pyridine hydrochloride (32 mg, 0.11 mmol, 1.0 equiv) and 2-(2-phenyl-1,3-thiazol-4-yl)acetic acid (25 mg, 0.11 mmol, 1.0 equiv) to afford 2-(2-phenyl-1,3-thiazol-4-yl)-1-(4-{[6-(trifluoromethyl)pyridin-2-yl]oxy}piperidin-1-yl)ethan-1-one (36 mg, 71%) as a light yellow oil. ¹H NMR (500 MHZ, CDCl₃) δ 8.26 (ddd, J=5.0, 1.9, 0.7 Hz, 1H), 7.94-7.87 (m, 2H), 7.84 (ddd, J=7.6, 2.0, 0.8 Hz, 1H), 7.44-7.36 (m, 3H), 7.17 (d, J=0.9 Hz, 1H), 6.94 (ddd, J=7.5, 5.0, 0.8 Hz, 1H), 5.50-5.44 (m, 1H), 4.03-3.90 (m, 3H), 3.78 (dtt, J=22.7, 13.5, 4.4 Hz, 2H), 3.55 (ddd, J=13.4, 8.1, 5.2 Hz, 1H), 1.89 (dq, J=14.1, 4.3 Hz, 4H). MS m/z: 448.5 [M+H]⁺.

### 2-(2-Phenyl-1,3-thiazol-4-yl)-1-[4-(pyridin-4-yloxy)piperidin-1-yl]ethan-1-one (403)

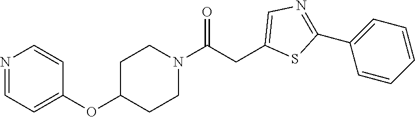 [SMILES: O=C(Cc1cnc(-c2ccccc2)s1)N1CCC(Oc2ccncc2)CC1]

Followed General Procedure E using 4-(piperidin-4-yloxy)pyridine (20 mg, 0.11 mmol, 1.0 equiv) and 2-(2-phenyl-1,3-thiazol-4-yl)acetic acid (25 mg, 0.11 mmol, 1.0 equiv) to afford 2-(2-phenyl-1,3-thiazol-4-yl)-1-[4-(pyridin-4-yloxy)piperidin-1-yl]ethan-1-one (29 mg, 67%) as colorless oil. ¹H NMR (500 MHZ, CDCl₃) δ 8.44-8.39 (m, 2H), 7.95-7.88 (m, 2H), 7.48-7.39 (m, 3H), 7.19 (s, 1H), 6.83-6.76 (m, 2H), 4.65 (tt, J=6.6, 3.5 Hz, 1H), 4.06-3.93 (m, 2H), 3.88 (ddd, J=13.7, 8.5, 3.6 Hz, 1H), 3.80-3.72 (m, 3H), 1.98-1.78 (m, 4H). MS m/z: 380.5 [M+H]⁺.

### 4-Phenoxy-1-(1-phenylcyclopentanecarbonyl)piperidine (404)

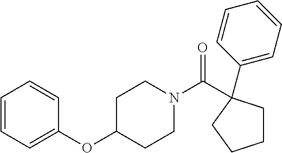 [SMILES: O=C(N1CCC(Oc2ccccc2)CC1)C1(c2ccccc2)CCCC1]

Followed General Procedure E using 4-phenoxypiperidine (20 mg, 0.11 mmol, 1.0 equiv) and 1-phenylcyclopentane-1-carboxylic acid (22 mg, 0.11 mmol, 1.0 equiv) to afford 4-phenoxy-1-(1-phenylcyclopentanecarbonyl)piperidine (9 mg, 23%) as a colorless oil. ¹H NMR (500 MHZ, CDCl₃) δ 7.31 (t, J=7.6 Hz, 2H), 7.25-7.17 (m, 5H), 6.91 (t, J=7.3 Hz, 1H), 6.82 (d, J=8.0 Hz, 2H), 4.36 (dq, J=6.7, 3.3 Hz, 1H), 3.82 (s, 1H), 3.66 (s, 1H), 3.30 (s, 1H), 3.02 (s, 1H), 2.43 (s, 2H), 2.04-1.85 (m, 4H), 1.75 (br s, 4H), 1.26 (s, 2H). MS m/z: 350.2 [M+H]⁺

### 2-(4-Phenoxypiperidine-1-carbonyl)quinoxaline (405)

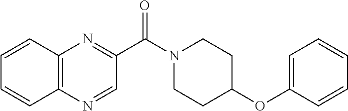 [SMILES: O=C(c1cnc2ccccc2n1)N1CCC(Oc2ccccc2)CC1]

Followed General Procedure E using 4-phenoxypiperidine (20 mg, 0.11 mmol, 1.0 equiv) and quinoxaline-2-carboxylic acid (20 mg, 0.11 mmol, 1.0 equiv) to afford 2-(4-phenoxypiperidine-1-carbonyl)quinoxaline (28 mg, 73%) as a light yellow oil. ¹H NMR (500 MHz, CDCl₃) δ 9.18 (d, J=1.4 Hz, 1H), 8.15 (dt, J=7.4, 1.7 Hz, 1H), 8.09 (dt, J=7.8, 1.6 Hz, 1H), 7.84 (ddd, J=6.7, 4.5, 1.9 Hz, 2H), 7.31 (tt, J=7.2, 1.5 Hz, 2H), 7.02-6.92 (m, 3H), 4.68 (dt, J=6.3, 3.1 Hz, 1H), 4.03 (dt, J=13.7, 5.3 Hz, 1H), 4.00-3.85 (m, 2H), 3.71 (ddd, J=13.8, 6.2, 4.2 Hz, 1H), 2.16-1.99 (m, 3H), 1.99-1.88 (m, 1H). MS m/z: 334.2 [M+H]⁺.

### 1-(2,3-Dihydro-1,4-benzodioxine-6-carbonyl)-4-phenoxypiperidine (406)

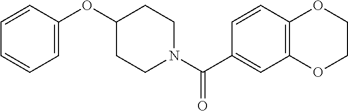 [SMILES: O=C(c1ccc2c(c1)OCCO2)N1CCC(Oc2ccccc2)CC1]

Followed General Procedure E using 4-phenoxypiperidine (20 mg, 0.11 mmol, 1.0 equiv) and 2,3-dihydro-1,4-benzodioxine-6-carboxylic acid (20 mg, 0.11 mmol, 1.0 equiv) to afford 1-(2,3-dihydro-1,4-benzodioxine-6-carbonyl)-4-phenoxypiperidine (38 mg, 93%) as a colorless oil. ¹H NMR (500 MHZ, CDCl₃) δ 7.32-7.27 (m, 2H), 6.99-6.94 (m, 2H), 6.92 (d, J=8.3 Hz, 3H), 6.89-6.85 (m, 1H), 4.58 (dt, J=6.6, 3.4 Hz, 1H), 4.28 (s, 4H), 3.76-3.45 (m, 4H), 1.88 (s, 4H). MS m/z: 340.2 [M+H]⁺.

### 4-Phenoxy-1-(5,6,7,8-tetrahydronaphthalene-2-carbonyl)piperidine (407)

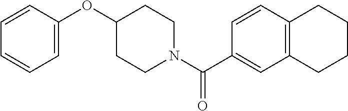 [SMILES: O=C(c1ccc2c(c1)CCCC2)N1CCC(Oc2ccccc2)CC1]

Followed General Procedure E using 4-phenoxypiperidine (20 mg, 0.11 mmol, 1.0 equiv) and 5,6,7,8-tetrahydronaphthalene-2-carboxylic acid (20 mg, 0.11 mmol, 1.0 equiv) to afford 4-phenoxy-1-(5,6,7,8-tetrahydronaphthalene-2-carbonyl)piperidine (32 mg, 85%) as a white solid. ¹H NMR (500 MHZ, CDCl₃) δ 7.31-7.26 (m, 2H), 7.13 (d, J=1.8 Hz, 1H), 7.11 (dd, J=7.7, 1.8 Hz, 1H), 7.07 (d, J=7.8 Hz, 1H), 6.96 (tt, J=7.3, 1.1 Hz, 1H), 6.94-6.90 (m, 2H), 4.57 (tt, J=6.8, 3.5 Hz, 1H), 3.92-3.41 (m, 4H), 2.77 (ddd, J=6.7, 4.1, 2.1 Hz, 4H), 2.09-1.83 (m, 4H), 1.80 (dq, J=6.6, 3.0 Hz, 4H). MS m/z: 336.2 [M+H]⁺.

### 1-(4-Phenoxypiperidin-1-yl)-2-(2-phenyl-1,3-thiazol-4-yl)ethan-1-one (408)

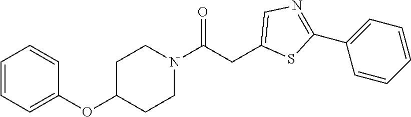 [SMILES: O=C(Cc1cnc(-c2ccccc2)s1)N1CCC(Oc2ccccc2)CC1]

Followed General Procedure E using 4-phenoxypiperidine (20 mg, 0.11 mmol, 1.0 equiv) and 2-(2-phenyl-1,3-thiazol-4-yl)acetic acid (25 mg, 0.11 mmol, 1.0 equiv) to afford 1-(4-phenoxypiperidin-1-yl)-2-(2-phenyl-1,3-thiazol-4-yl)ethan-1-one (32 mg, 85%) as a colorless oil. ¹H NMR (500 MHZ, CDCl₃) δ 7.96-7.89 (m, 2H), 7.47-7.41 (m, 3H), 7.31-7.26 (m, 2H), 7.18 (s, 1H), 6.96 (tt, J=7.3, 1.1 Hz, 1H), 6.92-6.88 (m, 2H), 4.54 (tt, J=6.5, 3.5 Hz, 1H), 4.03-3.93 (m, 2H), 3.88 (ddd, J=13.6, 8.4, 3.7 Hz, 1H), 3.82-3.64 (m, 3H), 1.86 (m, 4H). MS m/z: 378.2 [M+H]⁺.

### 1-(7-Methyl-1-benzofuran-2-carbonyl)-4-phenoxypiperidine (409)

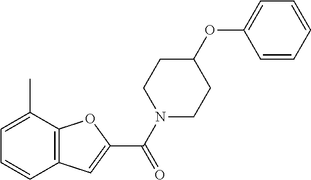 [SMILES: Cc1cccc2cc(C(=O)N3CCC(Oc4ccccc4)CC3)oc12]

Followed General Procedure E using 4-phenoxypiperidine (20 mg, 0.11 mmol, 1.0 equiv) and 7-methyl-1-benzofuran-2-carboxylic acid (20 mg, 0.11 mmol, 1.0 equiv) to afford 1-(7-methyl-1-benzofuran-2-carbonyl)-4-phenoxypiperidine (33 mg, 97%) as a colorless oil. ¹H NMR (500 MHZ, CDCl₃) δ 7.45 (t, J=4.6 Hz, 1H), 7.31-7.26 (m, 2H), 7.23 (d, J=0.7 Hz, 1H), 7.16 (d, J=4.7 Hz, 2H), 6.98-6.89 (m, 3H), 4.62 (tt, J=6.4, 3.4 Hz, 1H), 4.12-3.78 (m, 4H), 2.50 (s, 3H), 2.07-1.88 (m, 4H). MS m/z: 378.2 [M+H]⁺.

### 4-Phenoxy-1-(2-phenyl-2H-1,2,3-triazole-4-carbonyl)piperidine (410)

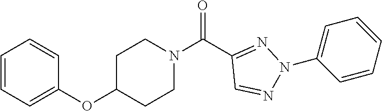 [SMILES: O=C(c1cnn(-c2ccccc2)n1)N1CCC(Oc2ccccc2)CC1]

Followed General Procedure E using 4-phenoxypiperidine (20 mg, 0.11 mmol, 1.0 equiv) and 2-phenyl-2H-1,2,3-triazole-4-carboxylic acid (21 mg, 0.11 mmol, 1.0 equiv) to afford 4-phenoxy-1-(2-phenyl-2H-1,2,3-triazole-4-carbonyl)piperidine (35 mg, 99%) as a white solid. ¹H NMR (500 MHZ, CDCl₃) δ 8.21 (s, 1H), 8.10-8.04 (m, 2H), 7.54-7.47 (m, 2H), 7.44-7.36 (m, 1H), 7.35-7.28 (m, 2H), 7.01-6.92 (m, 3H), 4.66 (tt, J=6.5, 3.4 Hz, 1H), 4.21 (ddd, J=12.6, 8.6, 3.6 Hz, 1H), 4.11 (ddd, J=13.7, 6.6, 4.0 Hz, 1H), 4.00-3.86 (m, 2H), 2.06 (ddq, J=12.6, 6.9, 3.4 Hz, 2H), 1.97 (dd, J=13.4, 6.2 Hz, 2H). MS m/z: 349.2 [M+H]⁺.

### 2-(3-Methoxyphenoxy)-1-(4-phenoxypiperidin-1-yl)ethan-1-one (411)

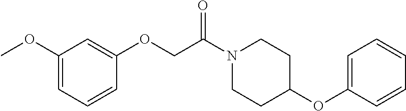 [SMILES: COc1cccc(OCC(=O)N2CCC(Oc3ccccc3)CC2)c1]

Followed General Procedure E using 4-phenoxypiperidine (20 mg, 0.11 mmol, 1.0 equiv) and 2-(3-methoxyphenoxy)acetic acid (21 mg, 0.11 mmol, 1.0 equiv) to afford 2-(3-methoxyphenoxy)-1-(4-phenoxypiperidin-1-yl)ethan-1-one (31 mg, 99%) as a colorless oil. ¹H NMR (500 MHZ, CDCl₃) δ 7.29 (td, J=7.4, 1.2 Hz, 2H), 7.19 (t, J=8.1 Hz, 1H), 6.96 (tt, J=7.4, 1.0 Hz, 1H), 6.93-6.87 (m, 2H), 6.58-6.50 (m, 3H), 4.69 (d, J=2.0 Hz, 2H), 4.56 (tt, J=6.4, 3.4 Hz, 1H), 3.79 (s, 3H), 3.78-3.68 (m, 3H), 3.55 (ddd, J=13.6, 6.4, 4.1 Hz, 1H), 1.88 (dtt, J=24.8, 13.8, 4.6 Hz, 4H). MS m/z: 341.2 [M+H]⁺.

### 6-(4-Phenoxypiperidine-1-carbonyl)-1H-indole (412)

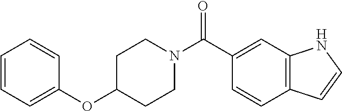 [SMILES: O=C(c1ccc2cc[nH]c2c1)N1CCC(Oc2ccccc2)CC1]

Followed General Procedure E using 4-phenoxypiperidine (20 mg, 0.11 mmol, 1.0 equiv) and 1H-indole-6-carboxylic acid (18 mg, 0.11 mmol, 1.0 equiv) to afford 6-(4-phenoxypiperidine-1-carbonyl)-1H-indole (28 mg, 97%) as a white solid. ¹H NMR (500 MHz, CDCl₃) δ 8.56 (s, 1H), 7.64 (d, J=8.1 Hz, 1H), 7.55 (s, 1H), 7.32-7.27 (m, 3H), 7.16 (dd, J=8.1, 1.4 Hz, 1H), 6.96 (td, J=7.4, 1.0 Hz, 1H), 6.95-6.91 (m, 2H), 6.57 (ddd, J=3.1, 2.0, 0.9 Hz, 1H), 4.59 (tt, J=6.8, 3.5 Hz, 1H), 4.10-3.31 (m, 4H), 2.16-1.68 (m, 4H). MS m/z: 321.2 [M+H]⁺.

### 4-Phenoxy-1-[3-(propan-2-yl)-1H-pyrazole-5-carbonyl]piperidine (413)

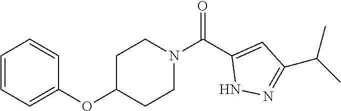 [SMILES: CC(C)c1cc(C(=O)N2CCC(Oc3ccccc3)CC2)[nH]n1]

Followed General Procedure E using 4-phenoxypiperidine (20 mg, 0.11 mmol, 1.0 equiv) and 3-(propan-2-yl)-1H-pyrazole-5-carboxylic acid (17 mg, 0.11 mmol, 1.0 equiv) to afford 4-phenoxy-1-[3-(propan-2-yl)-1H-pyrazole-5-carbonyl]piperidine (28 mg, 97%) as a white solid. ¹H NMR (500 MHZ, CDCl₃) δ 7.31-7.27 (m, 2H), 6.96 (tt, J=8.1, 0.9 Hz, 1H), 6.93 (dt, J=7.8, 1.1 Hz, 2H), 6.41-6.33 (m, 1H), 4.60 (tt, J=6.6, 3.4 Hz, 1H), 4.14-3.76 (m, 4H), 3.02 (pd, J=7.0, 0.7 Hz, 1H), 2.81 (s, 1H), 1.95 (d, J=46.5 Hz, 4H), 1.30 (s, 3H), 1.29 (s, 3H). MS m/z: 314.2 [M+H]⁺.

### 2-(4-Phenoxypiperidin-1-yl)quinoxaline (414)

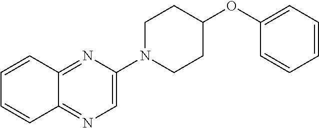 [SMILES: c1ccc(OC2CCN(c3cnc4ccccc4n3)CC2)cc1]

Followed General Procedure C using 4-phenoxypiperidine (35 mg, 0.2 mmol, 1.0 equiv) and 2-chloroquinoxaline (33 mg, 0.2 mmol, 1.0 equiv) to afford 2-(4-phenoxypiperidin-1-yl)quinoxaline (46 mg, 76%) as a light yellow solid. ¹H NMR (500 MHZ, DMSO-d₆) δ 8.87 (s, 1H), 7.83 (d, J=9.2 Hz, 1H), 7.64-7.56 (m, 2H), 7.40 (ddd, J=8.2, 6.2, 2.0 Hz, 1H), 7.31 (t, J=7.7 Hz, 2H), 7.02 (d, J=8.8 Hz, 2H), 6.95 (t, J=7.3 Hz, 1H), 4.71 (tt, J=7.8, 3.7 Hz, 1H), 4.25-4.12 (m, 2H), 3.62 (ddd, J=12.9, 8.9, 3.4 Hz, 2H), 2.13-2.02 (m, 2H), 1.76-1.62 (m, 2H). MS m/z: 306.2 [M+H]⁺.

### 6-[4-(Benzyloxy)piperidine-1-carbonyl]-1H-indole (415)

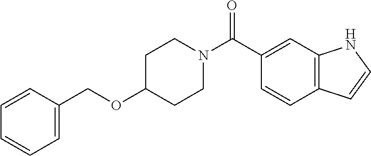 [SMILES: O=C(c1ccc2cc[nH]c2c1)N1CCC(OCc2ccccc2)CC1]

Followed General Procedure E using 4-(benzyloxy)piperidine hydrochloride (25 mg, 0.11 mmol, 1.0 equiv) and 1H-indole-6-carboxylic acid (18 mg, 0.11 mmol, 1.0 equiv) to afford 6-[4-(benzyloxy)piperidine-1-carbonyl]-1H-indole (35 mg, 96%) as a white solid. ¹H NMR (500 MHZ, CDCl₃) δ 8.52 (s, 1H), 7.54 (d, J=8.1 Hz, 1H), 7.43 (dd, J=1.5, 0.7 Hz, 1H), 7.27 (s, 1H), 7.23-7.18 (m, 2H), 7.17 (s, 1H), 7.04 (dd, J=8.1, 1.4 Hz, 1H), 6.52-6.41 (m, 1H), 4.49 (s, 2H), 4.18-3.65 (m, 2H), 3.60 (tt, J=7.6, 3.6 Hz, 1H), 3.38-3.22 (m, 2H), 2.01-1.56 (m, 4H). MS m/z: 335.2 [M+H]⁺.

### 6-[4-(Phenoxymethyl)piperidine-1-carbonyl]-1H-indole (416)

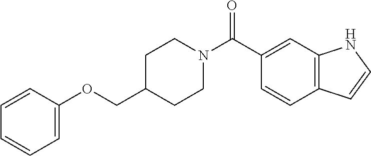 [SMILES: O=C(c1ccc2cc[nH]c2c1)N1CCC(COc2ccccc2)CC1]

Followed General Procedure E using 4-(phenoxymethyl)piperidine hydrochloride (25 mg, 0.11 mmol, 1.0 equiv) and 1H-indole-6-carboxylic acid (18 mg, 0.11 mmol, 1.0 equiv) to afford 6-[4-(phenoxymethyl)piperidine-1-carbonyl]-1H-indole (24 mg, 65%) as a white solid. ¹H NMR (500 MHZ, CDCl₃) δ 8.47 (br s, 1H), 7.64 (d, J=8.1 Hz, 1H), 7.54 (q, J=1.1 Hz, 1H), 7.31-7.26 (m, 3H), 7.16 (dd, J=8.1, 1.4 Hz, 1H), 6.95 (tt, J=7.3, 1.1 Hz, 1H), 6.91-6.85 (m, 2H), 6.57 (ddd, J=3.1, 2.0, 1.0 Hz, 1H), 4.81 (s, 1H), 4.05 (d, J=65.4 Hz, 1H), 3.84 (s, 2H), 2.95 (d, J=68.6 Hz, 2H), 2.16-2.03 (m, 1H), 1.91 (s, 2H), 1.40 (s, 2H). MS m/z: 335.2 [M+H]⁺.

### 2-[4-(Benzyloxy)piperidin-1-yl]quinoxaline (417)

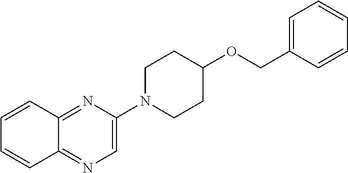 [SMILES: c1ccc(COC2CCN(c3cnc4ccccc4n3)CC2)cc1]

Followed General Procedure C using 4-(benzyloxy)piperidine hydrochloride (28 mg, 0.12 mmol, 1.0 equiv) and 2-chloroquinoxaline (20 mg, 0.12 mmol, 1.0 equiv) to afford 2-[4-(benzyloxy)piperidin-1-yl]quinoxaline (30 mg, 77%) as a white solid. ¹H NMR (500 MHz, CDCl₃) δ 8.49 (s, 1H), 7.75 (dd, J=8.3, 1.5 Hz, 1H), 7.55 (dd, J=8.3, 1.4 Hz, 1H), 7.45 (ddd, J=8.4, 6.9, 1.5 Hz, 1H), 7.29-7.26 (m, 1H), 7.26-7.22 (m, 3H), 7.18 (ddd, J=7.8, 4.5, 2.2 Hz, 1H), 7.14 (s, 1H), 4.50 (s, 2H), 4.06 (ddd, J=13.4, 6.8, 3.9 Hz, 2H), 3.62 (tt, J=7.7, 3.7 Hz, 1H), 3.46-3.35 (m, 2H), 1.98-1.89 (m, 2H), 1.72-1.65 (m, 2H). MS m/z: 320.2 [M+H]⁺.

### 2-[4-(Phenoxymethyl)piperidin-1-yl]quinoxaline (418)

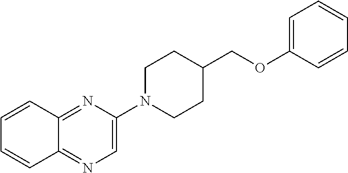 [SMILES: c1ccc(OCC2CCN(c3cnc4ccccc4n3)CC2)cc1]

Followed General Procedure C using 4-(phenoxymethyl)piperidine hydrochloride (28 mg, 0.12 mmol, 1.0 equiv) and 2-chloroquinoxaline (20 mg, 0.12 mmol, 1.0 equiv) to 2-[4-(phenoxymethyl)piperidin-1-yl]quinoxaline (27 mg, 70%) as a white solid. ¹H NMR (500 MHz, CDCl₃) δ 8.61 (s, 1H), 7.87 (dd, J=8.3, 1.4 Hz, 1H), 7.68 (dd, J=8.3, 1.3 Hz, 1H), 7.57 (ddt, J=8.2, 6.8, 1.1 Hz, 1H), 7.38 (ddt, J=8.2, 6.9, 1.2 Hz, 1H), 7.29 (tt, J=7.4, 0.9 Hz, 2H), 6.95 (td, J=7.3, 1.1 Hz, 1H), 6.90 (dq, J=7.1, 1.1 Hz, 2H), 4.65 (dt, J=13.4, 2.8 Hz, 2H), 3.86 (d, J=6.4 Hz, 2H), 3.07 (td, J=12.9, 2.7 Hz, 2H), 2.20-2.12 (m, 1H), 2.08-1.98 (m, 2H), 1.48 (qd, J=12.5, 4.3 Hz, 2H). MS m/z: 320.2 [M+H]⁺.

### Biological Assay Data and Procedures

Exemplary compounds were evaluated for activation of GCase in a live cell PFB assay in HELA cells (essentially as described in Ysselstein et al., “LRRK2 kinase activity regulates lysosomal glucocerebrosidase in neurons derived from Parkinson's disease patients” Nature Communications (2019) 10:5570). The results in Table 3 demonstrate that compounds of the disclosure are potent activators of GCase. EC₅₀ ranges: A: <10 μM; B: >10-50 μM; C: >50-100 μM; D: >100 μM.

TABLE 3
In vitro enzymatic EC₅₀ values for exemplary compounds

| Compound | GCase EC₅₀ (μM) |
|---|---|
| 1 | A |
| 3 | B |
| 4 | A |
| 5b | B |
| 5c | A |
| 6 | A |
| 7 | B |
| 8 | B |
| 9 | B |
| 10 | B |
| 11 | B |
| 12 | B |
| 13 | A |
| 14 | B |
| 15 | B |
| 16 | B |
| 17 | B |
| 18 | B |
| 19 | B |
| 20 | A |
| 21 | B |
| 22 | B |
| 23 | B |
| 24 | B |
| 25 | B |
| 26 | A |
| 30 | D |
| 31 | A |
| 33 | B |
| 35 | B |
| 36 | B |
| 38 | B |
| 39 | B |
| 40 | B |
| 41 | D |
| 42 | B |
| 43 | B |
| 44 | D |
| 45a | A |
| 45b | A |
| 46 | B |
| 47a | A |
| 47b | A |
| 48 | A |
| 48a | D |
| 48b | D |
| 48c | B |
| 48d | B |
| 49 | B |
| 50 | B |
| 51 | D |
| 52 | B |
| 55 | A |
| 58 | B |
| 59 | A |
| 60 | A |
| 61 | B |
| 62 | B |
| 63 | B |
| 64 | B |
| 65 | A |
| 66 | B |
| 67 | B |
| 68 | B |
| 69 | D |
| 71 | A |
| 72 | B |
| 73 | A |
| 74 | B |
| 75 | B |
| 76 | B |
| 77 | A |
| 78 | D |
| 79 | A |
| 80 | A |
| 81 | B |
| 84 | B |
| 86 | B |
| 88 | B |
| 89 | B |
| 90 | B |
| 91 | B |
| 92 | B |
| 93 | A |
| 94 | A |
| 95 | A |
| 96 | B |
| 97 | D |
| 98 | D |
| 99 | B |
| 100 | B |
| 101 | B |
| 103 | B |
| 105 | D |
| 108 | C |
| 109 | B |
| 110 | C |
| 111 | A |
| 112 | A |
| 113 | A |
| 115 | B |
| 116 | A |
| 117 | A |
| 118 | A |
| 119 | B |
| 120 | C |
| 121 | B |
| 122 | A |
| 123 | A |
| 124 | B |
| 126 | A |
| 127 | A |
| 128 | B |
| 129 | B |
| 132 | A |
| 133 | D |
| 135 | B |
| 137 | D |
| 140 | B |
| 141 | B |
| 142 | A |
| 147 | B |
| 150 | B |
| 151 | B |
| 152 | B |
| 153 | B |
| 155 | B |
| 156 | B |
| 161 | B |
| 163 | B |
| 170 | B |
| 176 | B |
| 177 | B |
| 178 | A |
| 181 | B |
| 182 | B |
| 187 | B |
| 188 | D |
| 194 | B |
| 197 | B |
| 198 | B |
| 199 | B |
| 204 | B |
| 207 | A |
| 209 | B |
| 213 | B |
| 217 | B |
| 218 | B |
| 219 | B |
| 220 | C |
| 222 | D |
| 223 | D |
| 224 | B |
| 225 | D |
| 226 | B |
| 227 | A |
| 228 | B |
| 229a | A |
| 229b | A |
| 230a | B |
| 230b | A |
| 231 | A |
| 232 | A |
| 233b | A |
| 234b | B |
| 235 | B |
| 236 | B |
| 239 | B |
| 247 | D |
| 248 | A |
| 252b | A |
| 255 | B |
| 257 | C |
| 258 | C |
| 260 | B |
| 261 | D |
| 262b | A |
| 263 | B |
| 264 | B |
| 266a | B |
| 266b | B |
| 269 | D |
| 272 | B |
| 273 | B |
| 274 | A |
| 275 | D |
| 276 | C |
| 277 | B |
| 278 | B |
| 279 | A |
| 280 | D |
| 281 | A |
| 282 | A |
| 283 | A |
| 284 | B |
| 285 | B |
| 286 | D |
| 288a | A |
| 288b | B |
| 289 | A |
| 290 | B |
| 291 | A |
| 292 | B |
| 295 | A |
| 296 | A |
| 297 | B |
| 298a | C |
| 299a | B |
| 299b | A |
| 300 | B |
| 306 | D |
| 307 | B |
| 308 | A |
| 309 | B |
| 310 | B |
| 311 | A |
| 312 | A |
| 313 | B |
| 314 | C |
| 315 | A |
| 316 | A |
| 317 | B |
| 318 | B |
| 319 | B |
| 320 | B |
| 321 | B |
| 322 | C |
| 323 | A |
| 324 | B |
| 326 | B |
| 327 | B |
| 328 | B |
| 329 | A |
| 332 | B |
| 333 | B |
| 335 | B |
| 336 | B |
| 340 | B |
| 350 | C |
| 351 | B |
| 359 | B |
| 360 | B |
| 369 | B |
| 370 | A |
| 375 | B |
| 379 | D |
| 381 | B |
| 383 | B |
| 386 | B |
| 387 | B |
| 391 | B |
| 393 | B |
| 399 | B |
| 400 | B |
| 401 | B |
| 402 | B |
| 404 | B |
| 407 | B |
| 408 | B |
| 409 | B |
| 410 | B |
| 414 | B |
| 415 | C |
| 416 | B |
| 417 | B |
| 418 | B |
| 419 | D |
| 420 | B |
| 421 | B |
| 422 | B |
| 423 | C |
| 424 | A |
| 425 | D |
| 426 | B |
| 427 | B |
| 428 | B |
| 429 | D |
| 430 | C |
| 431 | C |
| 432 | C |
| 433 | B |
| 434 | D |
| 435 | D |
| 436 | D |
| 437 | B |
| 438 | B |
| 439 | C |
| 440 | D |
| 441 | D |
| 442 | D |
| 443 | C |
| 444 | D |
| 445 | D |
| 446 | B |
| 447 | D |
| 448 | B |
| 449 | D |
| 450 | B |
| 451 | D |
| 452 | D |
| 453 | D |
| 454 | D |
| 455 | B |
| 456 | A |
| 457 | D |
| 458 | D |
| 459 | B |
| 460 | C |
| 461 | B |
| 462 | D |
| 463 | C |
| 464 | B |
| 465 | C |
| 466 | D |
| 467 | D |
| 468 | C |
| 469 | D |
| 470 | D |
| 471 | C |
| 472 | C |
| 473 | D |
| 474 | D |
| 475 | B |
| 476 | C |
| 477 | A |
| 478 | C |
| 479 | B |
| 480 | D |
| 481 | B |
| 482 | D |
| 483 | C |
| 484 | D |
| 485 | B |
| 486 | B |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

For reasons of completeness, various aspects of the present disclosure are set out in the following numbered clauses:

Clause 1. A compound of Formula (I):

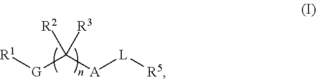

or a pharmaceutically acceptable salt thereof, wherein:
R¹ is substituted or unsubstituted heteroaryl, or substituted or unsubstituted aryl;
G is —O— or —CR²R³—;
R² and R³ are each independently hydrogen, halogen, or substituted or unsubstituted alkyl;
n is 1 or 0;
A is

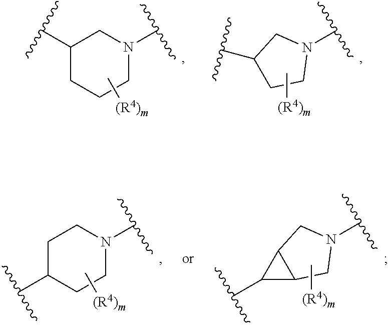

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted heteroaryl, substituted or unsubstituted heterocyclyl, substituted or unsubstituted heteroarylalkyl, substituted or unsubstituted carbocyclyl, or substituted or unsubstituted aryloxyalkyl.

Clause 2. A compound of Formula (I):

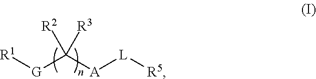

or a pharmaceutically acceptable salt thereof, wherein:
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

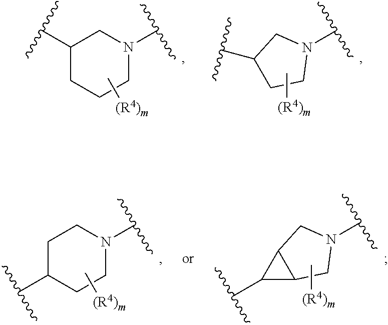

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted heteroarylalkyl, substituted or unsubstituted cycloalkyl, or substituted or unsubstituted aryloxyalkyl.

Clause 3. The compound of clause 1 or 2, or a pharmaceutically acceptable salt thereof, wherein:
R¹ is substituted pyridinyl, or substituted or unsubstituted phenyl.

Clause 4. The compound of any of clauses 1-3, or a pharmaceutically acceptable salt thereof, wherein:
R¹ is pyridinyl substituted with haloalkyl or haloalkoxy, unsubstituted phenyl, or phenyl substituted with haloalkyl or alkyl.

Clause 5. The compound of any of clauses 1-4, or a pharmaceutically acceptable salt thereof, wherein:
R¹ is pyridinyl substituted with haloalkyl or haloalkoxy.

Clause 6. The compound of any of clauses 1-5, or a pharmaceutically acceptable salt thereof, wherein:
R¹ is pyridinyl substituted with haloalkyl.

Clause 7. The compound of any of clauses 1-4, or a pharmaceutically acceptable salt thereof, wherein:
R¹ is unsubstituted phenyl.

Clause 8. The compound of any of clauses 1-4, or a pharmaceutically acceptable salt thereof, wherein:
R¹ is phenyl substituted with haloalkyl or alkyl.

Clause 9. The compound of any of clauses 1-4, or a pharmaceutically acceptable salt thereof, wherein:
R¹ is phenyl substituted with haloalkyl.

Clause 10. The compound of any of clauses 1-4, or a pharmaceutically acceptable salt thereof, wherein:
R¹ is

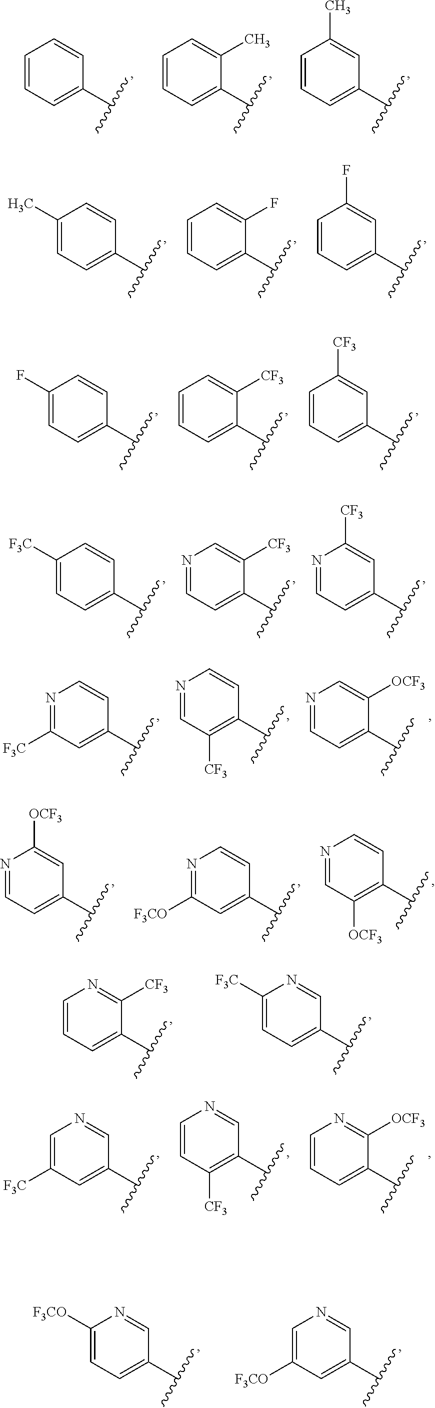

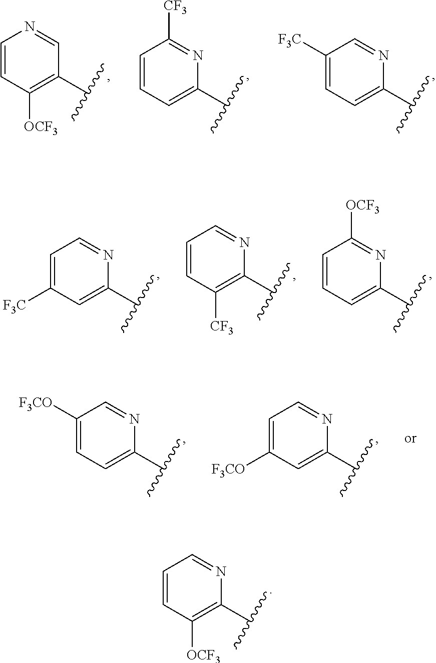

Clause 11. The compound of any of clauses 1-4, or a pharmaceutically acceptable salt thereof, wherein:
R¹ is

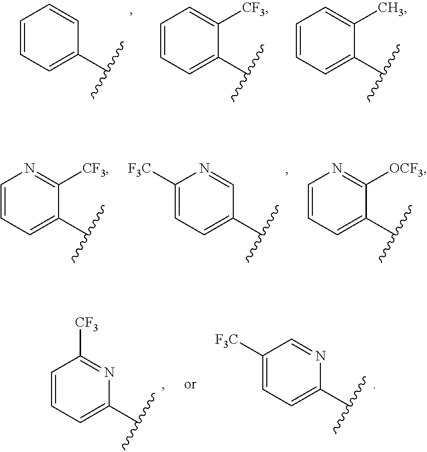

Clause 12. The compound of any of clauses 1-4, or a pharmaceutically acceptable salt thereof, wherein:
R¹ is

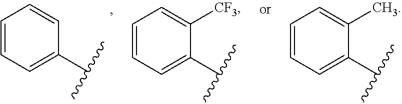 [SMILES: *c1ccccc1 ; *c1ccccc1C(F)(F)F ; *c1ccccc1C]

Clause 13. The compound of any of clauses 1-4, or a pharmaceutically acceptable salt thereof, wherein:
R¹ is

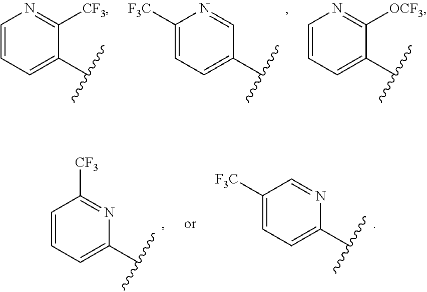 [SMILES: *c1cccnc1C(F)(F)F ; *c1ccc(C(F)(F)F)nc1 ; *c1cccnc1OC(F)(F)F ; *c1cccc(C(F)(F)F)n1 ; *c1ccc(C(F)(F)F)cn1]

Clause 14. The compound of any of clauses 1-13, or a pharmaceutically acceptable salt thereof, wherein:
G is —O—.

Clause 15. The compound of any of clauses 1-13, or a pharmaceutically acceptable salt thereof, wherein:
G is —CR²R³—.

Clause 16. The compound of any of clauses 1-13, or a pharmaceutically acceptable salt thereof, wherein:
G is —CH₂— or —CH(CH₃)—.

Clause 17. The compound of any of clauses 1-13, or a pharmaceutically acceptable salt thereof, wherein:
G is —CH₂—.

Clause 18. The compound of any of clauses 1-13, or a pharmaceutically acceptable salt thereof, wherein:
G is —CH(CH₃)—.

Clause 19. The compound of any of clauses 1-18, or a pharmaceutically acceptable salt thereof, wherein:
n is 1.

Clause 20. The compound of any of clauses 1-18, or a pharmaceutically acceptable salt thereof, wherein:
n is 0.

Clause 21. The compound of any of clauses 1-18, or a pharmaceutically acceptable salt thereof, wherein:
when n is 0, then A is

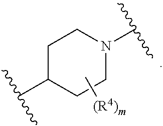

Clause 22. The compound of any of clauses 1-20, or a pharmaceutically acceptable salt thereof, wherein:
A is

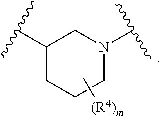

Clause 23. The compound of any of clauses 1-20, or a pharmaceutically acceptable salt thereof, wherein:
A is

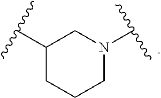 [SMILES: *C1CCCN(*)C1]

Clause 24. The compound of any of clauses 1-20, or a pharmaceutically acceptable salt thereof, wherein:
A is

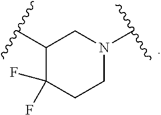 [SMILES: *C1CN(*)CCC1(F)F]

Clause 25. The compound of any of clauses 1-20, or a pharmaceutically acceptable salt thereof, wherein:
A is

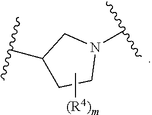

Clause 26. The compound of any of clauses 1-20, or a pharmaceutically acceptable salt thereof, wherein:
A is

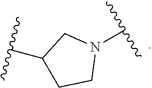 [SMILES: *C1CCN(*)C1]

Clause 27. The compound of any of clauses 1-20, or a pharmaceutically acceptable salt thereof, wherein:
A is

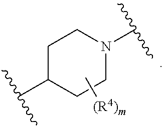

Clause 28. The compound of any of clauses 1-20, or a pharmaceutically acceptable salt thereof, wherein:
A is

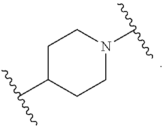 [SMILES: *C1CCN(*)CC1]

Clause 29. The compound of any of clauses 1-20, or a pharmaceutically acceptable salt thereof, wherein:
A is

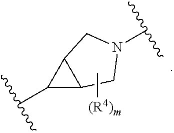

Clause 30. The compound of any of clauses 1-20, or a pharmaceutically acceptable salt thereof, wherein:
A is

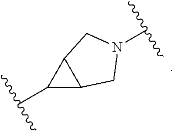 [SMILES: *C1C2CN(*)CC12]

Clause 31. The compound of any of clauses 1-20, or a pharmaceutically acceptable salt thereof, wherein:
A is

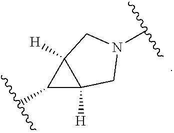 [SMILES: *[C@@H]1[C@H]2CN(*)C[C@@H]12]

Clause 32. The compound of any of clauses 1-31, or a pharmaceutically acceptable salt thereof, wherein:
R⁴ is halogen, or two instances of R⁴ on the same carbon form with that carbon a carbonyl.

Clause 33. The compound of any of clauses 1-31, or a pharmaceutically acceptable salt thereof, wherein:
R⁴ is fluoro, or two instances of R⁴ on the same carbon form with that carbon a carbonyl.

Clause 34. The compound of any of clauses 1-31, or a pharmaceutically acceptable salt thereof, wherein:
R⁴ is fluoro.

Clause 35. The compound of any of clauses 1-34, or a pharmaceutically acceptable salt thereof, wherein:
m is 0.

Clause 36. The compound of any of clauses 1-34, or a pharmaceutically acceptable salt thereof, wherein:
m is 2.

Clause 37. The compound of any of clauses 1-36, or a pharmaceutically acceptable salt thereof, wherein:
L is a bond.

Clause 38. The compound of any of clauses 1-36, or a pharmaceutically acceptable salt thereof, wherein:
L is —C(═O)—.

Clause 39. The compound of any of clauses 1-38, or a pharmaceutically acceptable salt thereof, wherein:
R⁵ is substituted or unsubstituted pyrazolopyrazinyl, substituted or unsubstituted pyrrolopyrazinyl, substituted or unsubstituted chromenonyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted pyrazolylmethyl, substituted or unsubstituted indolylmethyl, substituted or unsubstituted cyclohexyl, or substituted or unsubstituted phenyloxyalkyl.

Clause 40. The compound of any of clauses 1-39, or a pharmaceutically acceptable salt thereof, wherein:
R⁵ is substituted pyrazolopyrazinyl, substituted pyrrolopyrazinyl, substituted chromenonyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl.

Clause 41. The compound of any of clauses 1-40, or a pharmaceutically acceptable salt thereof, wherein:
R⁵ is substituted pyrazolopyrazinyl, substituted pyrrolopyrazinyl, substituted chromenonyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl, wherein each substituted R⁵ is substituted with haloalkyl, cycloalkyl, heteroaryl, aryl, halogen, arylalkyl, alkoxy, alkyl, heterocyclylalkyl, or heterocyclyl.

Clause 42. The compound of any of clauses 1-41, or a pharmaceutically acceptable salt thereof, wherein:
R⁵ is

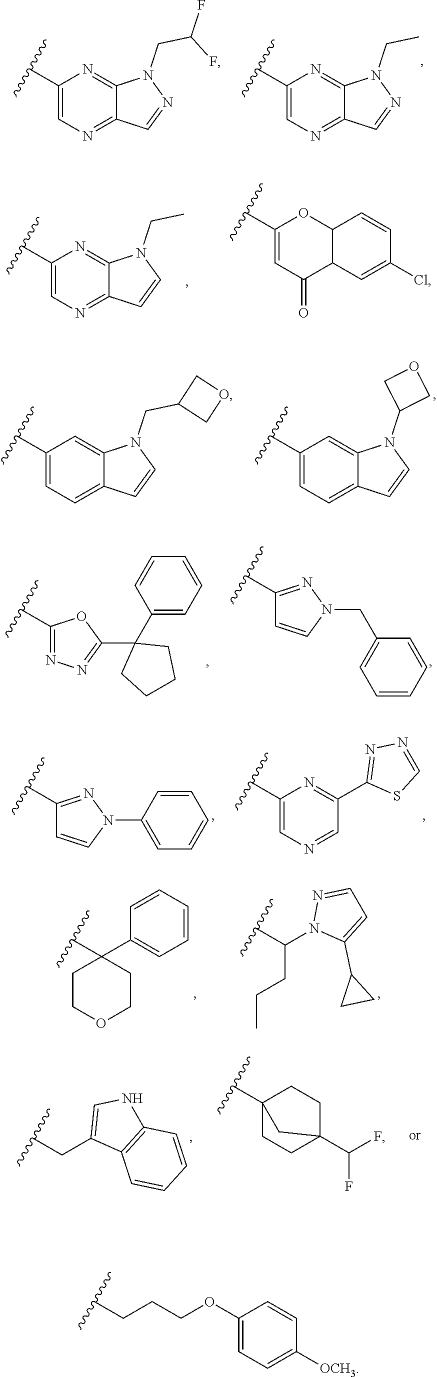

Clause 43. The compound of any of clauses 1-42, or a pharmaceutically acceptable salt thereof, wherein:
R⁵ is

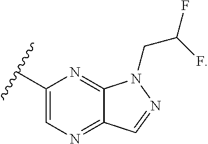 [SMILES: *c1cnc2cnn(CC(F)F)c2n1]

Clause 44. The compound of any of clauses 1-43, or a pharmaceutically acceptable salt thereof, wherein:
R² and R³ are each independently hydrogen, or substituted or unsubstituted alkyl.

Clause 45. The compound of any of clauses 1-44, or a pharmaceutically acceptable salt thereof, wherein:
R³ is hydrogen.

Clause 46. The compound of any of clauses 1-45, or a pharmaceutically acceptable salt thereof, wherein:
R³ is substituted or unsubstituted alkyl.

Clause 47. The compound of any of clauses 1-46, or a pharmaceutically acceptable salt thereof, wherein:
R³ is unsubstituted alkyl.

Clause 48. The compound of clause 1, wherein the compound is of formula (I-a):

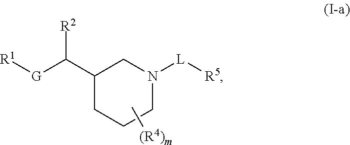

or a pharmaceutically acceptable salt thereof.

Clause 49. The compound of clause 1, wherein the compound is of formula (I-b):

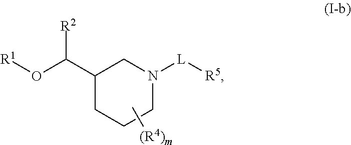

or a pharmaceutically acceptable salt thereof.

Clause 50. The compound of clause 1, wherein the compound is of formula (I-c):

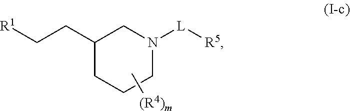

or a pharmaceutically acceptable salt thereof.

Clause 51. The compound of clause 1, wherein the compound is of formula (I-d):

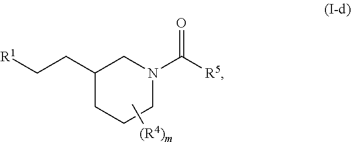

or a pharmaceutically acceptable salt thereof.

Clause 52. The compound of clause 1, wherein the compound is of formula (I-e):

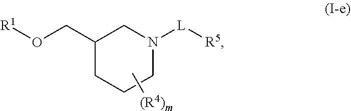

or a pharmaceutically acceptable salt thereof.

Clause 53. The compound of clause 1, wherein the compound is of formula (I-f):

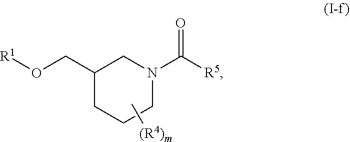

or a pharmaceutically acceptable salt thereof.

Clause 54. The compound of clause 1, wherein the compound is of formula (I-g):

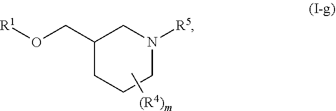

or a pharmaceutically acceptable salt thereof.

Clause 55. The compound of clause 1, wherein the compound is of formula (I-h):

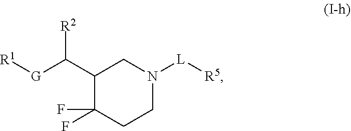

or a pharmaceutically acceptable salt thereof.

Clause 56. The compound of clause 1, wherein the compound is of formula (I-i):

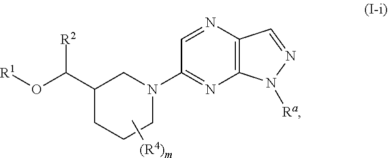

or a pharmaceutically acceptable salt thereof, wherein:
R² is hydrogen or alkyl; and
R^{a} is alkyl or haloalkyl.

Clause 57. The compound of clause 1, wherein the compound is of formula (II-a):

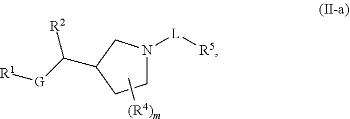

or a pharmaceutically acceptable salt thereof.

Clause 58. The compound of clause 1, wherein the compound is of formula (II-b):

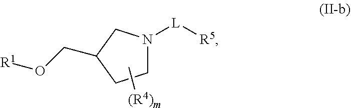

or a pharmaceutically acceptable salt thereof.

Clause 59. The compound of clause 1, wherein the compound is of formula (II-c):

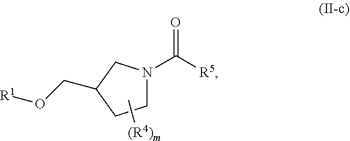

or a pharmaceutically acceptable salt thereof.

Clause 60. The compound of clause 1, wherein the compound is of formula (II-d):

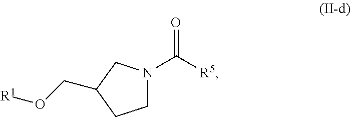

or a pharmaceutically acceptable salt thereof.

Clause 61. The compound of clause 1, wherein the compound is of formula (III-a):

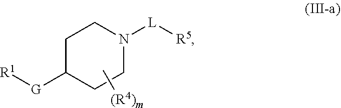

or a pharmaceutically acceptable salt thereof.

Clause 62. The compound of clause 1, wherein the compound is of formula (III-b):

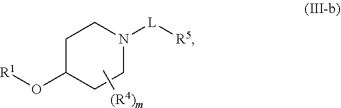

or a pharmaceutically acceptable salt thereof.

Clause 63. The compound of clause 1, wherein the compound is of formula (III-c):

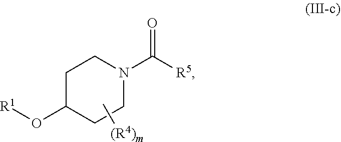

or a pharmaceutically acceptable salt thereof.

Clause 64. The compound of clause 1, wherein the compound is of formula (III-d):

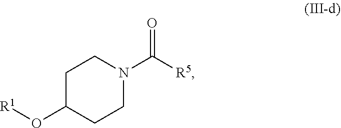

or a pharmaceutically acceptable salt thereof.

Clause 65. The compound of clause 1, wherein the compound is of formula (IV-a):

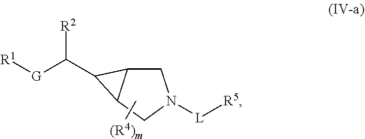

or a pharmaceutically acceptable salt thereof.

Clause 66. The compound of clause 1, wherein the compound is of formula (IV-b):

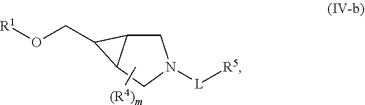

or a pharmaceutically acceptable salt thereof.

Clause 67. The compound of clause 1, wherein the compound is of formula (IV-c):

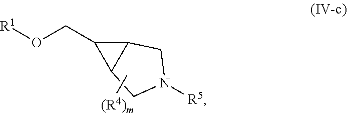

or a pharmaceutically acceptable salt thereof.

Clause 68. The compound of clause 1, wherein the compound is of formula (IV-d):

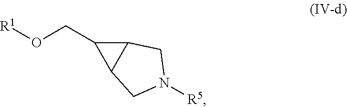

or a pharmaceutically acceptable salt thereof.

Clause 69. The compound of clause 1, wherein the compound is of formula (IV-e):

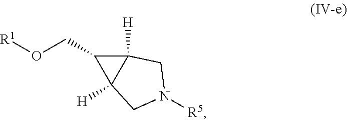

or a pharmaceutically acceptable salt thereof.

Clause 70. The compound of clause 1, wherein the compound is:

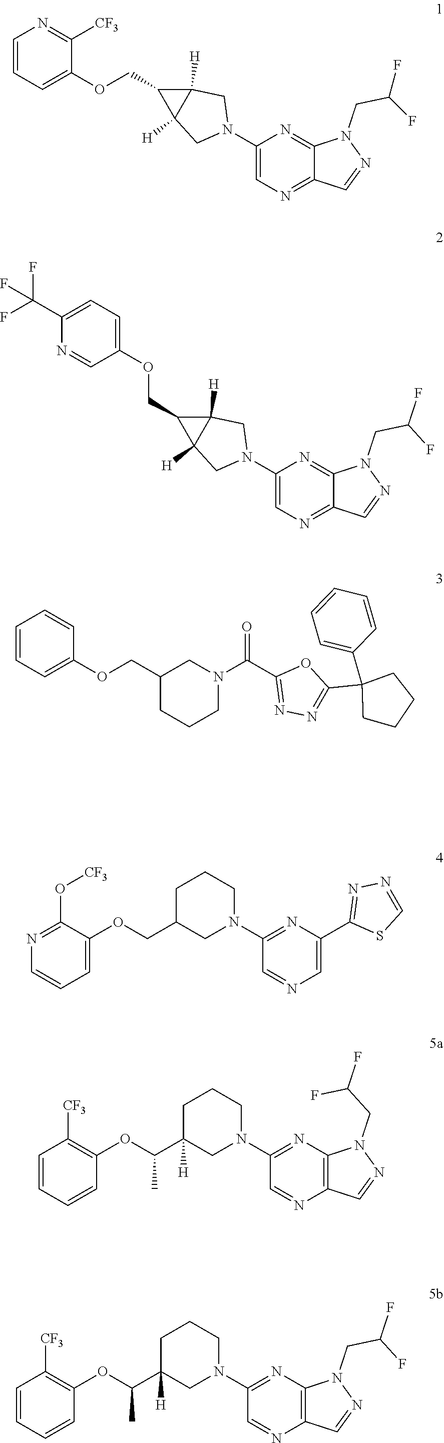 [SMILES: FC(F)Cn1ncc2ncc(N3C[C@@H]4[C@@H](COc5cccnc5C(F)(F)F)[C@@H]4C3)nc21 ; FC(F)Cn1ncc2ncc(N3C[C@@H]4[C@@H](COc5ccc(C(F)(F)F)nc5)[C@@H]4C3)nc21 ; O=C(c1nnc(C2(c3ccccc3)CCCC2)o1)N1CCCC(COc2ccccc2)C1 ; FC(F)(F)Oc1ncccc1OCC1CCCN(c2cncc(-c3nncs3)n2)C1 ; C[C@H](Oc1ccccc1C(F)(F)F)[C@H]1CCCN(c2cnc3cnn(CC(F)F)c3n2)C1 ; C[C@@H](Oc1ccccc1C(F)(F)F)[C@@H]1CCCN(c2cnc3cnn(CC(F)F)c3n2)C1]

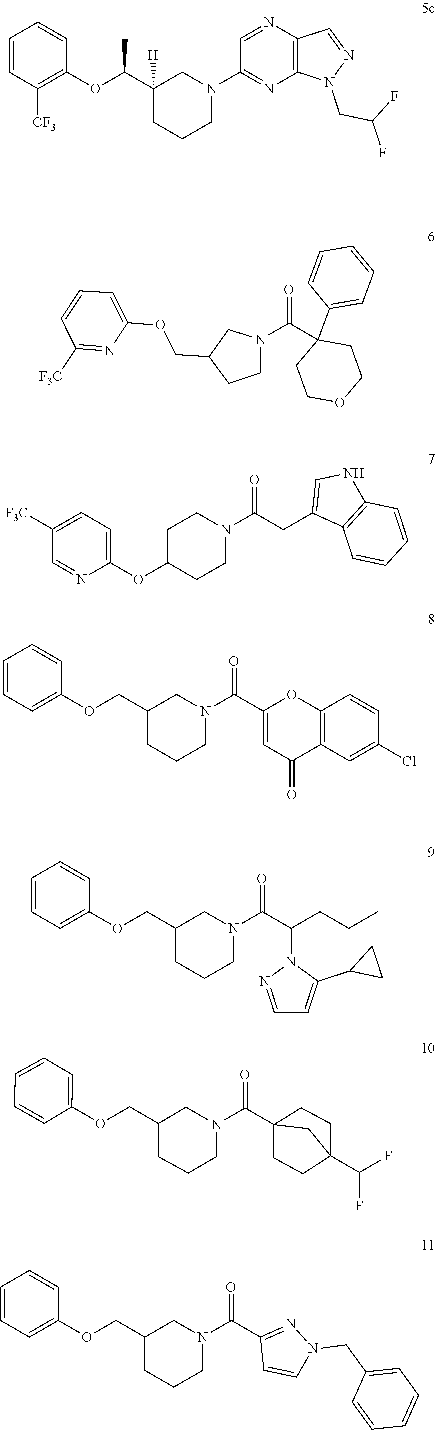

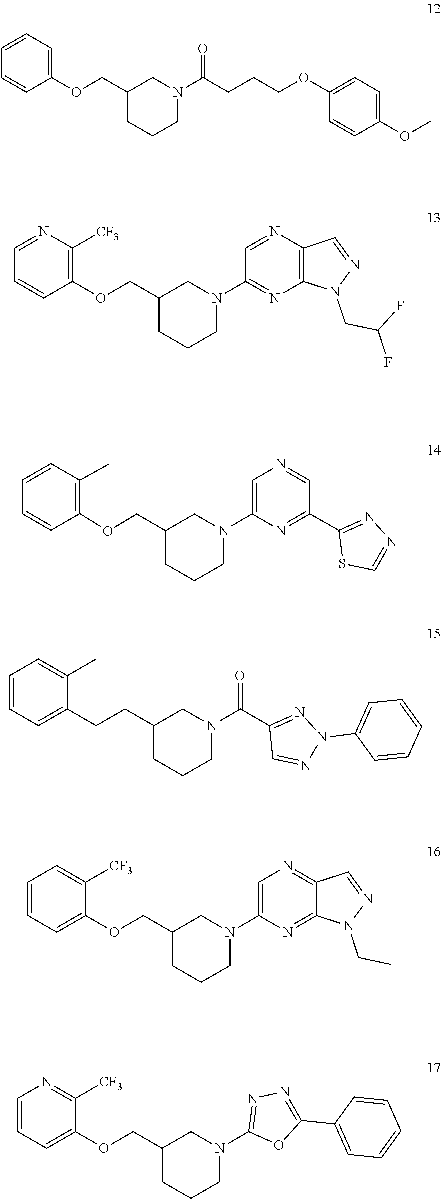 [SMILES: COc1ccc(OCCCC(=O)N2CCCC(COc3ccccc3)C2)cc1 ; FC(F)Cn1ncc2ncc(N3CCCC(COc4cccnc4C(F)(F)F)C3)nc21 ; Cc1ccccc1OCC1CCCN(c2cncc(-c3nncs3)n2)C1 ; Cc1ccccc1CCC1CCCN(C(=O)c2cnn(-c3ccccc3)n2)C1 ; CCn1ncc2ncc(N3CCCC(COc4ccccc4C(F)(F)F)C3)nc21 ; FC(F)(F)c1ncccc1OCC1CCCN(c2nnc(-c3ccccc3)o2)C1]

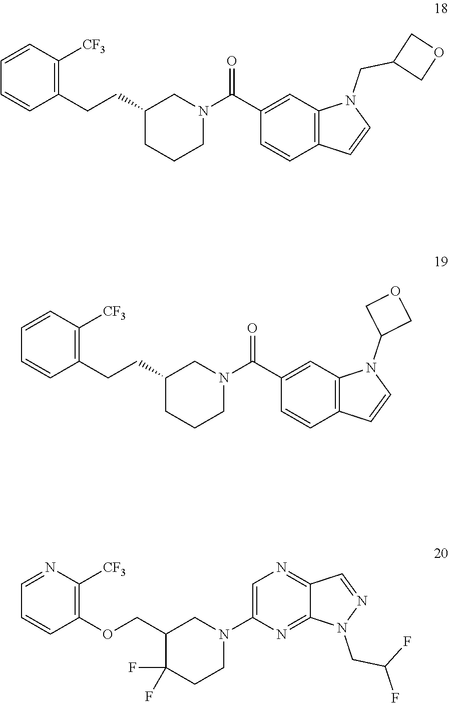 [SMILES: O=C(c1ccc2ccn(CC3COC3)c2c1)N1CCC[C@H](CCc2ccccc2C(F)(F)F)C1 ; O=C(c1ccc2ccn(C3COC3)c2c1)N1CCC[C@H](CCc2ccccc2C(F)(F)F)C1 ; FC(F)Cn1ncc2ncc(N3CCC(F)(F)C(COc4cccnc4C(F)(F)F)C3)nc21]

or a pharmaceutically acceptable salt thereof.

Clause 71. A pharmaceutical composition comprising a compound of any of clauses 1-70, or a pharmaceutically acceptable salt thereof, and a pharmaceutically acceptable excipient.

Clause 72. A kit comprising a compound of any of clauses 1-70, or a pharmaceutically acceptable salt thereof, or a pharmaceutical composition of clause 71, and instructions for administering the compound or pharmaceutical composition to a subject in need thereof.

Clause 73. A method of treating a disease or disorder in a subject in need thereof, the method comprising administering an effective amount of a compound of any of clauses 1-70, or pharmaceutically acceptable salt thereof, or a pharmaceutical composition of clause 71.

Clause 74. The method of clause 73, wherein the disease or disorder is associated with glucocerebrosidase activity.

Clause 75. The method of clause 73 or 74, wherein the disease or disorder is a neurological disease or disorder.

Clause 76. The method of clause 75, wherein the neurological disease or disorder is Parkinson's disease or Gaucher's disease.

Clause 77. A method of activating glucocerebrosidase, the method comprising contacting glucocerebrosidase with an effective amount of a compound of any of clauses 1-70, or pharmaceutically acceptable salt thereof, or a pharmaceutical composition of clause 71.

Clause 78. The method of clause 77, wherein the contacting is in vitro.

Clause 79. The method of clause 77, wherein the contacting is in vivo.

Additional various aspects of the present disclosure are set out in the following numbered embodiments:

Embodiment 1. A compound of Formula (I):

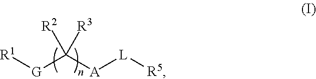

or a pharmaceutically acceptable salt thereof, wherein:
R¹ is substituted or unsubstituted heteroaryl, substituted or unsubstituted aryl, substituted or unsubstituted carbocyclyl, substituted or unsubstituted heterocyclyl, pentyl, butyl, methyl, —CH₂CH₂CH(CH₃)₂, or hydrogen, or optionally a heterocyclyl forming a spirocyclic ring system with A when n is 0 and G is a bond;
G is a bond, —S(O)₂—, —NR²—, —CH₂CH₂O—, —CH₂O—, —O— or —CR²R³—;
R² and R³ are each independently hydrogen, halogen, or substituted or unsubstituted alkyl, or R² and R³ on the same carbon form with that carbon a carbonyl;
n is 1 or 0;
A is

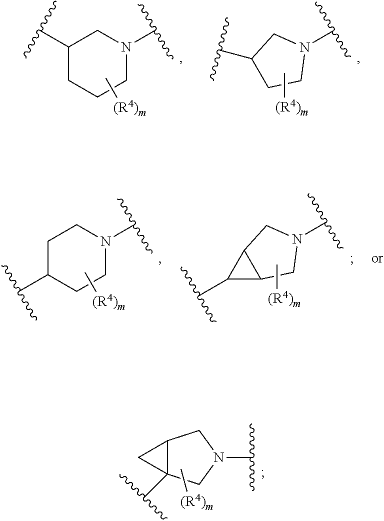

each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, —C(═O)CF₂—, —C(═O)CH(Ph)—, —C(═O)CH(iPr)—, —C(═O)CH(Et)—, —C(═O)CH(Me)—, —C(═O)C(CH₃)₂—, —C(═O)CH(OMe)—, —C(═O)CH₂CH₂—, —C(═O)CH₂CH₂CH₂—, —C(═O)CH₂CH₂CH₂O—, —C(═O)CH(CH₃)CH₂—, —C(═O)CH₂O—, —C(═O)CH₂OCH₂—, —C(═O)CH(CH₃)O—, —C(═O)CH₂CH═CH—, —C(═O)NHCH₂CH₂CH₂—, —C(═O)NHCH₂CH₂—, —CH₂—, —CH₂CH₂CH₂—, —CH₂C(CH₃)₂—, —C(═O)NH—, or —CH₂C(═O)NH—; and
R⁵ is substituted or unsubstituted heteroaryl, substituted or unsubstituted heterocyclyl, substituted or unsubstituted heteroarylalkyl, substituted or unsubstituted carbocyclyl, substituted or unsubstituted aryl, methyl, ethyl, butyl, pentyl, t-butyl, —CH₂CH₂CH(CH₃)₂, —SCF₃, or —OCH₂CH(CH₃)₂.

Embodiment 2. A compound of Formula (I):

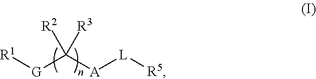

or a pharmaceutically acceptable salt thereof, wherein:
R¹ is substituted or unsubstituted heteroaryl, substituted or unsubstituted aryl, substituted or unsubstituted carbocyclyl, or substituted or unsubstituted heterocyclyl, pentyl, butyl, or
   —CH₂CH₂CH(CH₃)₂;
G is —S(O)₂—, —NR²—, —CH₂CH₂O—, —CH₂O—, —O— or —CR²R³—;
R² and R³ are each independently hydrogen, halogen, or substituted or unsubstituted alkyl;
n is 1 or 0;
A is

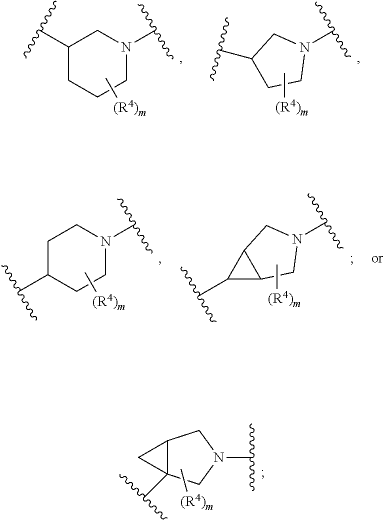

each R⁴ is independently halogen, substituted or unsubstituted alkyl, substituted or unsubstituted alkoxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, or —C(═O)CH₂O—; and
R⁵ is substituted or unsubstituted heteroaryl, substituted or unsubstituted heterocyclyl, substituted or unsubstituted heteroarylalkyl, substituted or unsubstituted aryl, substituted or unsubstituted carbocyclyl, or substituted or unsubstituted aryloxyalkyl.

Embodiment 3. A compound of Formula (I):

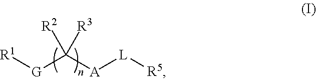

or a pharmaceutically acceptable salt thereof, wherein:
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is

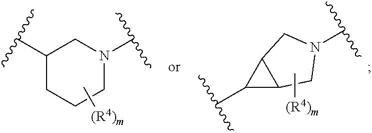

each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted indolyl, substituted or unsubstituted oxadiazolyl, substituted or unsubstituted triazolyl, or substituted or unsubstituted pyrazinyl.

Embodiment 4. The compound of any of embodiments 1-3, or a pharmaceutically acceptable salt thereof, wherein: R¹ is substituted or unsubstituted pyrazinyl, substituted or unsubstituted pyridinyl, or substituted or unsubstituted phenyl.

Embodiment 5. The compound of any of embodiments 1-3, or a pharmaceutically acceptable salt thereof, wherein: R¹ is substituted pyridinyl, or substituted or unsubstituted phenyl.

Embodiment 6. The compound of any of embodiments 1-3, or a pharmaceutically acceptable salt thereof, wherein: R¹ is pyridinyl substituted with haloalkyl or haloalkoxy, unsubstituted phenyl, or phenyl substituted with halogen, haloalkyl, or alkyl.

Embodiment 7. The compound of any of embodiments 1-4, or a pharmaceutically acceptable salt thereof, wherein: R¹ is pyridinyl substituted with haloalkyl or haloalkoxy.

Embodiment 8. The compound of any of embodiments 1-7, or a pharmaceutically acceptable salt thereof, wherein: R¹ is pyridinyl substituted with haloalkyl.

Embodiment 9. The compound of any of embodiments 1-6, or a pharmaceutically acceptable salt thereof, wherein: R¹ is unsubstituted phenyl.

Embodiment 10. The compound of any of embodiments 1-6, or a pharmaceutically acceptable salt thereof, wherein: R¹ is phenyl substituted with halogen, haloalkyl, or alkyl.

Embodiment 11. The compound of any of embodiments 1-6, or a pharmaceutically acceptable salt thereof, wherein: R¹ is phenyl substituted with haloalkyl.

Embodiment 12. The compound of any of embodiments 1-3, or a pharmaceutically acceptable salt thereof, wherein: R¹ is hydrogen, methyl, butyl, pentyl, —CH₂CH₂CH(CH₃)₂,

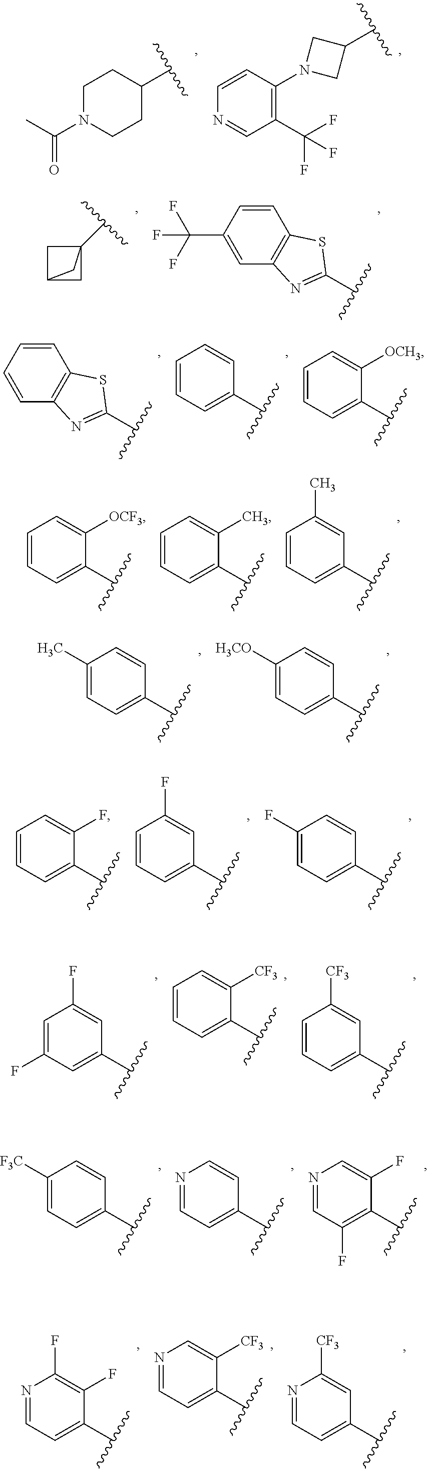

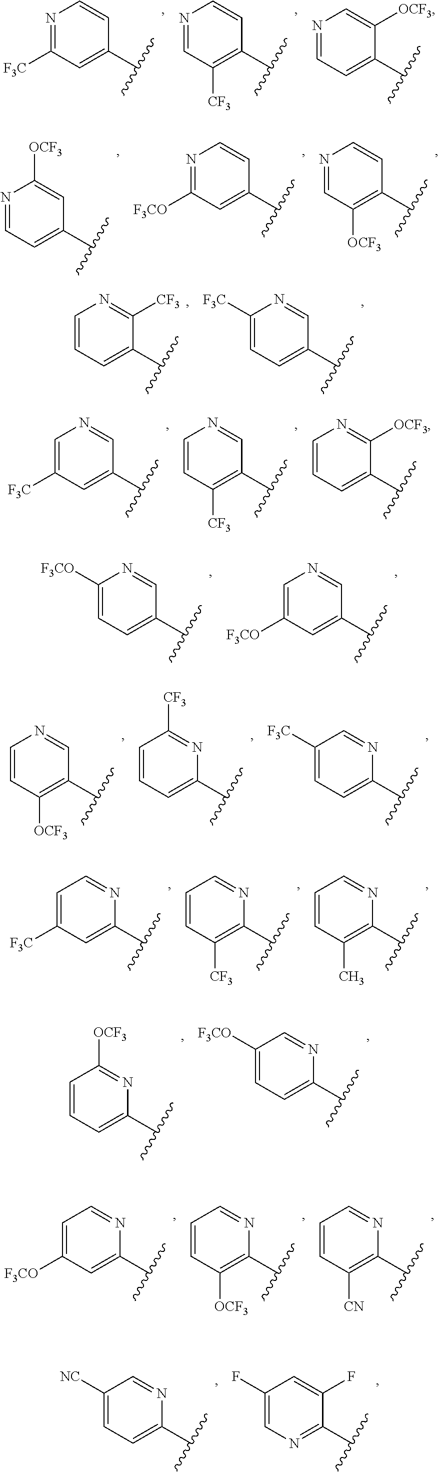

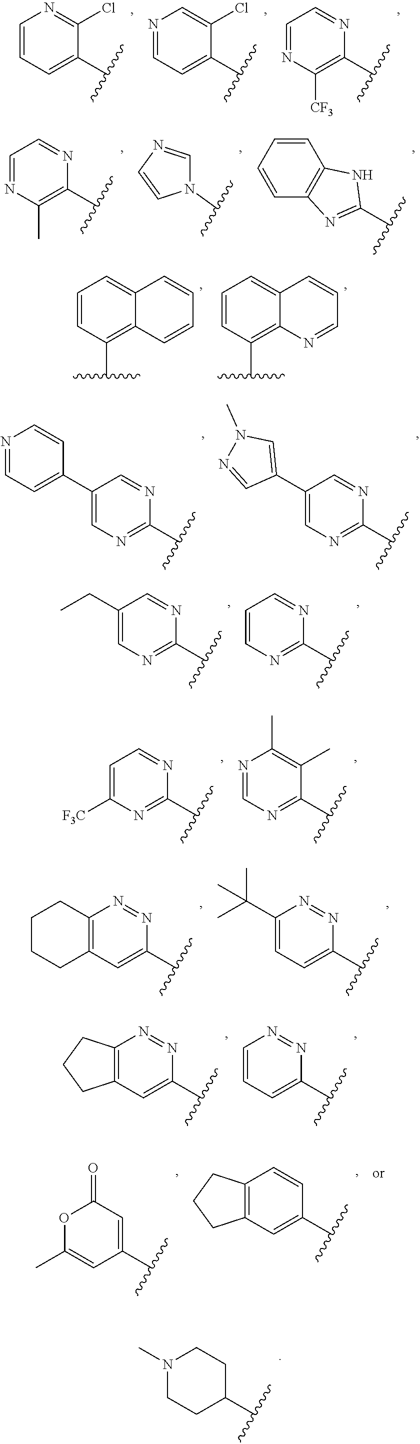

Embodiment 13. The compound of any of embodiments 1-3, or a pharmaceutically acceptable salt thereof, wherein: R¹ is butyl, pentyl, —CH₂CH₂CH(CH₃)₂.

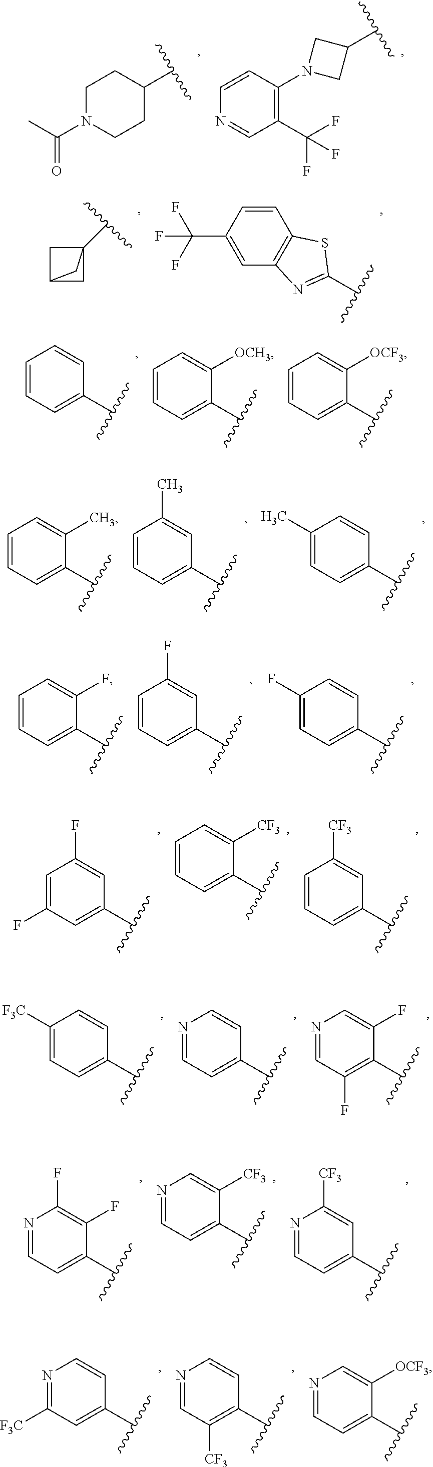

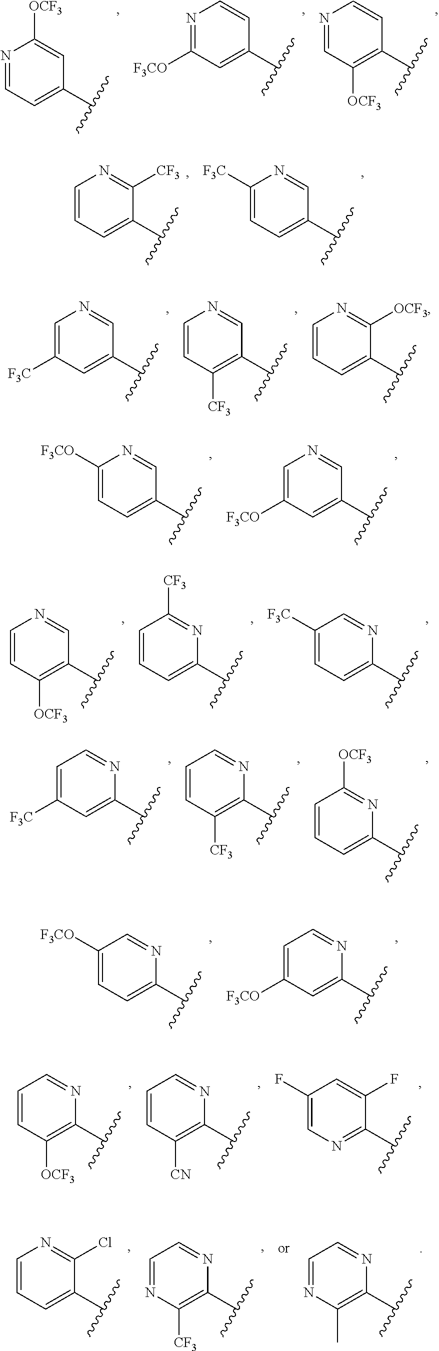

Embodiment 14. The compound of any of embodiments 1-6, or a pharmaceutically acceptable salt thereof, wherein: R¹ is

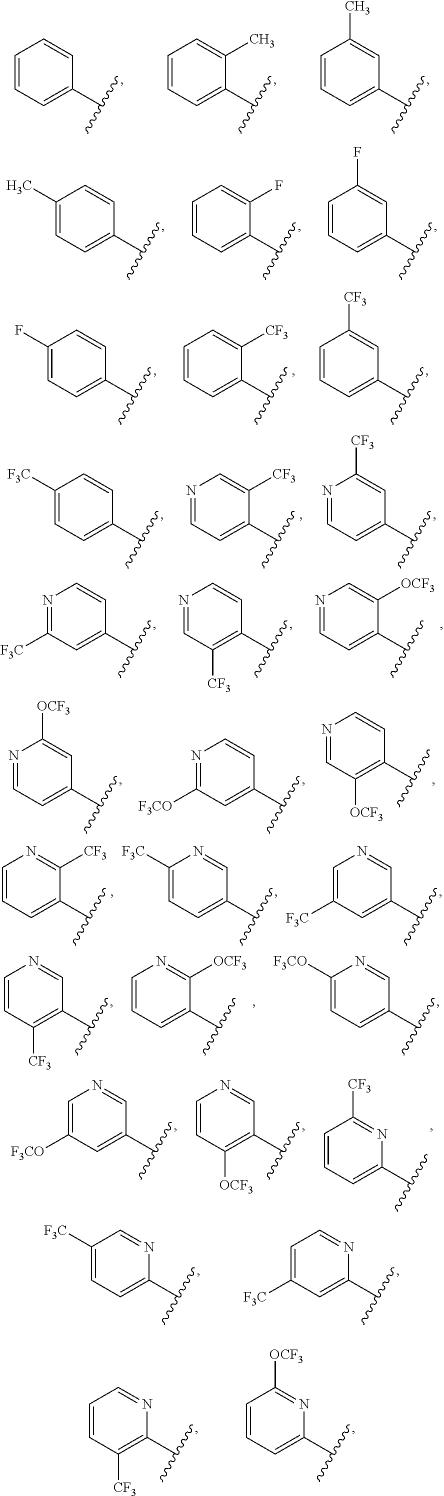

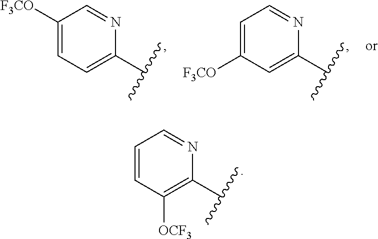 [SMILES: *c1ccc(OC(F)(F)F)cn1 ; *c1cc(OC(F)(F)F)ccn1 ; *c1ncccc1OC(F)(F)F]

Embodiment 15. The compound of any of embodiments 1-6, or a pharmaceutically acceptable salt thereof, wherein: R¹ is

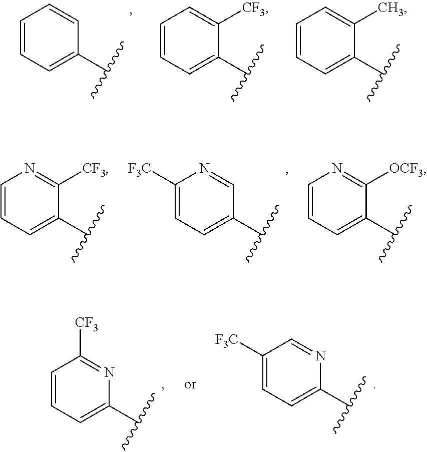

Embodiment 16. The compound of any of embodiments 1-6, or a pharmaceutically acceptable salt thereof, wherein: R¹ is

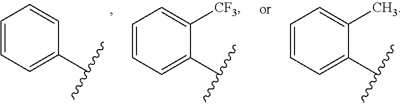 [SMILES: *c1ccccc1 ; *c1ccccc1C(F)(F)F ; *c1ccccc1C]

Embodiment 17. The compound of any of embodiments 1-6, or a pharmaceutically acceptable salt thereof, wherein: R¹ is

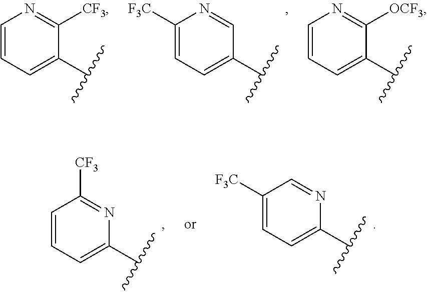 [SMILES: *c1cccnc1C(F)(F)F ; *c1ccc(C(F)(F)F)nc1 ; *c1cccnc1OC(F)(F)F ; *c1cccc(C(F)(F)F)n1 ; *c1ccc(C(F)(F)F)cn1]

Embodiment 18. The compound of any of embodiments 1-17, or a pharmaceutically acceptable salt thereof, wherein: G is —O—.

Embodiment 19. The compound of any of embodiments 1-17, or a pharmaceutically acceptable salt thereof, wherein: G is —CR²R³

Embodiment 20. The compound of any of embodiments 1-17, or a pharmaceutically acceptable salt thereof, wherein: G is —CH₂— or —CH(CH₃)—.

Embodiment 21. The compound of any of embodiments 1-17, or a pharmaceutically acceptable salt thereof, wherein: G is —CH₂—.

Embodiment 22. The compound of any of embodiments 1-17, or a pharmaceutically acceptable salt thereof, wherein: G is —CH(CH₃)—.

Embodiment 23. The compound of any of embodiments 1-22, or a pharmaceutically acceptable salt thereof, wherein: n is 1.

Embodiment 24. The compound of any of embodiments 1-22, or a pharmaceutically acceptable salt thereof, wherein: n is 0.

Embodiment 25. The compound of any of embodiments 1-24, or a pharmaceutically acceptable salt thereof, wherein: when n is 0, then A is

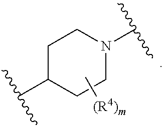

Embodiment 26. The compound of any of embodiments 1-24, or a pharmaceutically acceptable salt thereof, wherein: A is

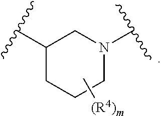

Embodiment 27. The compound of any of embodiments 1-24, or a pharmaceutically acceptable salt thereof, wherein: A is

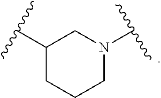 [SMILES: *C1CCCN(*)C1]

Embodiment 28. The compound of any of embodiments 1-24, or a pharmaceutically acceptable salt thereof, wherein: A is

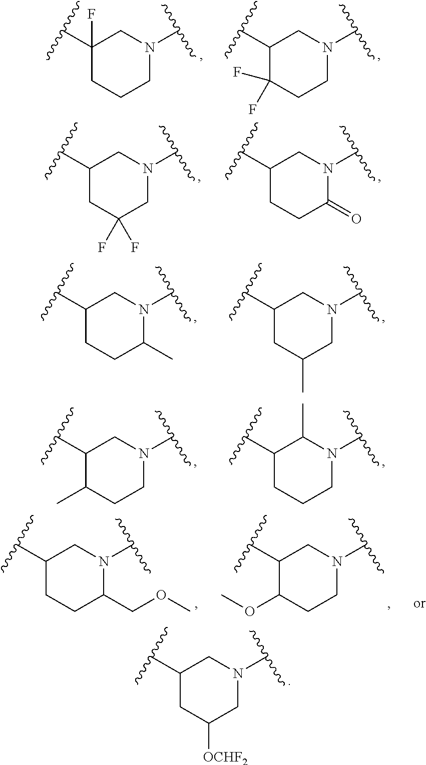

Embodiment 29. The compound of any of embodiments 1-24, or a pharmaceutically acceptable salt thereof, wherein: A is

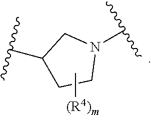

Embodiment 30. The compound of any of embodiments 1-24, or a pharmaceutically acceptable salt thereof, wherein: A is

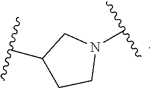 [SMILES: *C1CCN(*)C1]

Embodiment 31. The compound of any of embodiments 1-24, or a pharmaceutically acceptable salt thereof, wherein: A is

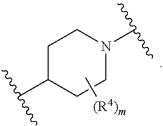

Embodiment 32. The compound of any of embodiments 1-24, or a pharmaceutically acceptable salt thereof, wherein: A is

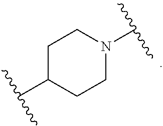 [SMILES: *C1CCN(*)CC1]

Embodiment 33. The compound of any of embodiments 1-24, or a pharmaceutically acceptable salt thereof, wherein: A is

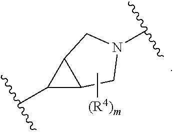

Embodiment 34. The compound of any of embodiments 1-24, or a pharmaceutically acceptable salt thereof, wherein: A is

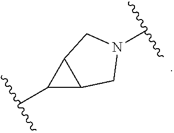 [SMILES: *C1C2CN(*)CC12]

Embodiment 35. The compound of any of embodiments 1-24, or a pharmaceutically acceptable salt thereof, wherein: A is

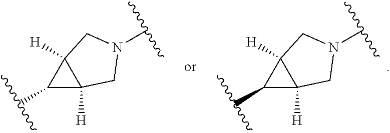 [SMILES: *[C@@H]1[C@H]2CN(*)C[C@@H]12 ; *[C@H]1[C@H]2CN(*)C[C@@H]12]

Embodiment 36. The compound of any of embodiments 1-24, or a pharmaceutically acceptable salt thereof, wherein: A is

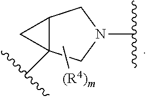

Embodiment 37. The compound of any of embodiments 1-24, or a pharmaceutically acceptable salt thereof, wherein: A is

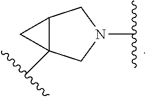 [SMILES: *N1CC2CC2(*)C1]

Embodiment 38. The compound of any of embodiments 1-37, or a pharmaceutically acceptable salt thereof, wherein: each R⁴ is independently halogen, or two instances of R⁴ on the same carbon form with that carbon a carbonyl.

Embodiment 39. The compound of any of embodiments 1-38, or a pharmaceutically acceptable salt thereof, wherein: each R⁴ is independently R⁴ is fluoro, or two instances of R⁴ on the same carbon form with that carbon a carbonyl.

Embodiment 40. The compound of any of embodiments 1-39, or a pharmaceutically acceptable salt thereof, wherein: R⁴ is fluoro.

Embodiment 41. The compound of any of embodiments 1-37, or a pharmaceutically acceptable salt thereof, wherein: each R⁴ is independently fluoro, methyl, CH₃OCH₂—, methoxy, difluoromethoxy, or two instances of R⁴ on the same carbon form with that carbon a carbonyl.

Embodiment 42. The compound of any of embodiments 1-41, or a pharmaceutically acceptable salt thereof, wherein: m is 0.

Embodiment 43. The compound of any of embodiments 1-41, or a pharmaceutically acceptable salt thereof, wherein: m is 2.

Embodiment 44. The compound of any of embodiments 1-43, or a pharmaceutically acceptable salt thereof, wherein: L is a bond.

Embodiment 45. The compound of any of embodiments 1-43, or a pharmaceutically acceptable salt thereof, wherein: L is —C(═O)—.

Embodiment 46. The compound of any of embodiments 1-43, or a pharmaceutically acceptable salt thereof, wherein: L is —C(═O)CH₂—.

Embodiment 47. The compound of any of embodiments 1-43 or a pharmaceutically acceptable salt thereof, wherein: L is —C(═O)CH₂O—.

Embodiment 48. The compound of any of embodiments 1-47, or a pharmaceutically acceptable salt thereof, wherein: R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted imidazopyridinyl, substituted or unsubstituted triazolopyridinyl, substituted or unsubstituted pyrazolopyrimidinyl, substituted or unsubstituted pyrrolopyrimidinyl, substituted or unsubstituted chromenonyl, substituted or unsubstituted isochromanyl, substituted or unsubstituted indolyl, substituted or unsubstituted quinoxalinyl, substituted or unsubstituted benzofuranyl, substituted or unsubstituted benzothiophenyl, substituted or unsubstituted benzimidazolyl, substituted or unsubstituted benzoxazolyl, substituted or unsubstituted 5,6,7,8-tetrahydro-[1,2,4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1,4]dioxinyl, substituted or unsubstituted tetrahydronaphthalenyl, substituted or unsubstituted isoquinolinonyl, substituted or unsubstituted quinolinyl, substituted or unsubstituted naphthyridinyl, substituted or unsubstituted naphthyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted imidazolyl, substituted or unsubstituted pyrrolyl, substituted or unsubstituted thiophenyl, substituted or unsubstituted furanyl, substituted or unsubstituted isothiazolyl, substituted or unsubstituted isoxazolyl, substituted or unsubstituted isoxazolonyl, substituted or unsubstituted 3,4-dihydro-1H-pyrrolo[2,1-c][1.4]thiazin-8-yl, substituted or unsubstituted pyrrolidinonyl, substituted or unsubstituted pyrrolidinyl, substituted or unsubstituted 1,4-diazepanyl, substituted or unsubstituted dioxolanonyl, substituted or unsubstituted pyridinyl, substituted or unsubstituted pyrazinyl, substituted or unsubstituted pyridazinyl, substituted or unsubstituted pyrimidinyl, substituted or unsubstituted phenyl, substituted or unsubstituted tetrahydropyranyl, substituted or unsubstituted tetrahydrofuranyl, substituted or unsubstituted morpholinyl, substituted or unsubstituted cyclooctyl, substituted or unsubstituted cyclohexyl, substituted or unsubstituted cyclopentyl, substituted or unsubstituted cyclobutyl, substituted or unsubstituted cyclopropyl, substituted or unsubstituted bicyclo[3.3.1]nonanyl, substituted or unsubstituted bicyclo[2.2.1]heptanyl, substituted or unsubstituted 7-oxaspiro[3.5]non-1-en-2-yl, substituted or unsubstituted hexahydro-1H-cyclopenta[c]furan-5-yl, substituted or unsubstituted adamantyl, substituted or unsubstituted spiro[2.5]octan-4-yl, methyl, ethyl, butyl, pentyl, t-butyl, —CH₂CH₂CH(CH₃)₂, —SCF₃, or —OCH₂CH(CH₃)₂.

Embodiment 49. The compound of any of embodiments 1-47, or a pharmaceutically acceptable salt thereof, wherein: R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted pyrazolopyrimidinyl, substituted or unsubstituted indolyl, substituted or unsubstituted quinoxalinyl, substituted or unsubstituted benzofuranyl, substituted or unsubstituted 5,6,7,8-tetrahydro-[1,2,4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1,4]dioxinyl, substituted or unsubstituted tetrahydronaphthalenyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted pyrazinyl, substituted or unsubstituted pyrimidinyl, substituted or unsubstituted phenyl, substituted or unsubstituted tetrahydropyranyl, substituted or unsubstituted morpholinyl, or substituted or unsubstituted cyclopentyl.

Embodiment 50. The compound of any of embodiments 1-49, or a pharmaceutically acceptable salt thereof, wherein: R⁵ is substituted or unsubstituted pyrazolopyrazinyl, substituted or unsubstituted pyrrolopyrazinyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted pyrazolylmethyl, substituted or unsubstituted indolylmethyl, substituted or unsubstituted cyclohexyl, or substituted or unsubstituted phenyloxyalkyl.

Embodiment 51. The compound of any of embodiments 1-50, or a pharmaceutically acceptable salt thereof, wherein: R⁵ is substituted pyrazolopyrazinyl, substituted pyrrolopyrazinyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl.

Embodiment 52. The compound of any of embodiments 1-51, or a pharmaceutically acceptable salt thereof, wherein: R⁵ is substituted pyrazolopyrazinyl, substituted pyrrolopyrazinyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl, wherein each substituted R⁵ is substituted with haloalkyl, cycloalkyl, heteroaryl, aryl, halogen, arylalkyl, alkoxy, alkyl, heterocyclylalkyl, or heterocyclyl.

Embodiment 53. The compound of any of embodiments 1-47, or a pharmaceutically acceptable salt thereof, wherein: R⁵ is

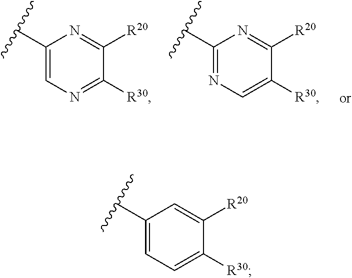

R²⁰ and R³⁰ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R²⁰ and R³⁰ together with the atoms to which they are attached form a substituted or unsubstituted carbocyclyl, substituted or unsubstituted heterocyclyl, substituted or unsubstituted aryl, or substituted or unsubstituted heteroaryl.

Embodiment 54. The compound of embodiment 53, or a pharmaceutically acceptable salt thereof, wherein: A is

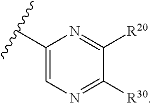

Embodiment 55. The compound of embodiment 53 or 54, or a pharmaceutically acceptable salt thereof, wherein: R²⁰ and R³⁰ are each independently hydrogen or substituted or unsubstituted heteroaryl; or R²⁰ and R³⁰ together with the atoms to which they are attached form a substituted or unsubstituted aryl, or substituted or unsubstituted heteroaryl.

Embodiment 56. The compound of any of embodiments 53-55, or a pharmaceutically acceptable salt thereof, wherein: R²⁰ is heteroaryl.

Embodiment 57. The compound of any of embodiments 53-56, or a pharmaceutically acceptable salt thereof, wherein: R²⁰ is thiadiazolyl.

Embodiment 58. The compound of any of embodiments 53-57, or a pharmaceutically acceptable salt thereof, wherein: R³⁰ is hydrogen.

Embodiment 59. The compound of any of embodiments 53-55, or a pharmaceutically acceptable salt thereof, wherein: R²⁰ and R³⁰ together with the atoms to which they are attached form a substituted or unsubstituted aryl, or substituted or unsubstituted heteroaryl.

Embodiment 60. The compound of any of embodiments 53-55, or a pharmaceutically acceptable salt thereof, wherein: R²⁰ and R³⁰ together with the atoms to which they are attached form a substituted or unsubstituted heteroaryl.

Embodiment 61. The compound of any of embodiments 53-55, or a pharmaceutically acceptable salt thereof, wherein: R²⁰ and R³⁰ together with the atoms to which they are attached form a substituted or unsubstituted imidazolyl, substituted or unsubstituted pyrrolyl, or a substituted or unsubstituted pyrazolyl.

Embodiment 62. The compound of any of embodiments 53-55, or a pharmaceutically acceptable salt thereof, wherein: R²⁰ and R³⁰ together with the atoms to which they are attached form a substituted or unsubstituted pyrazolyl.

Embodiment 63. The compound of any of embodiments 1-53, or a pharmaceutically acceptable salt thereof, wherein: R⁵ is

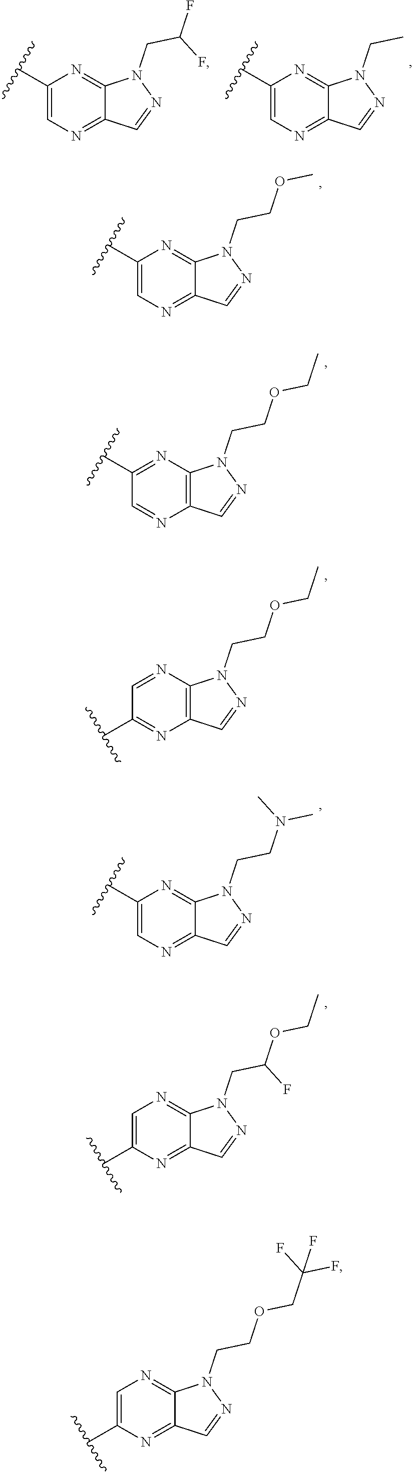

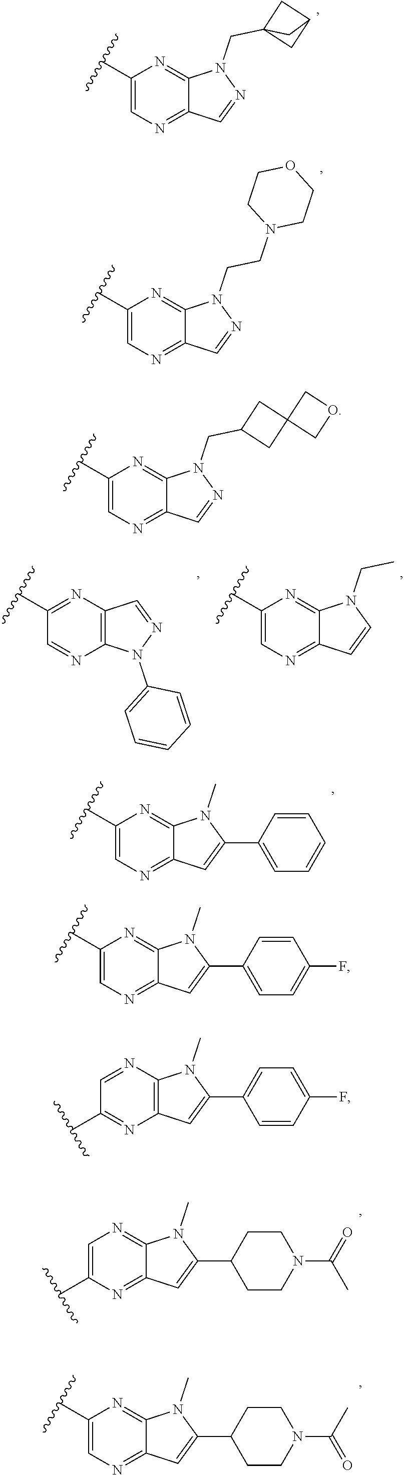

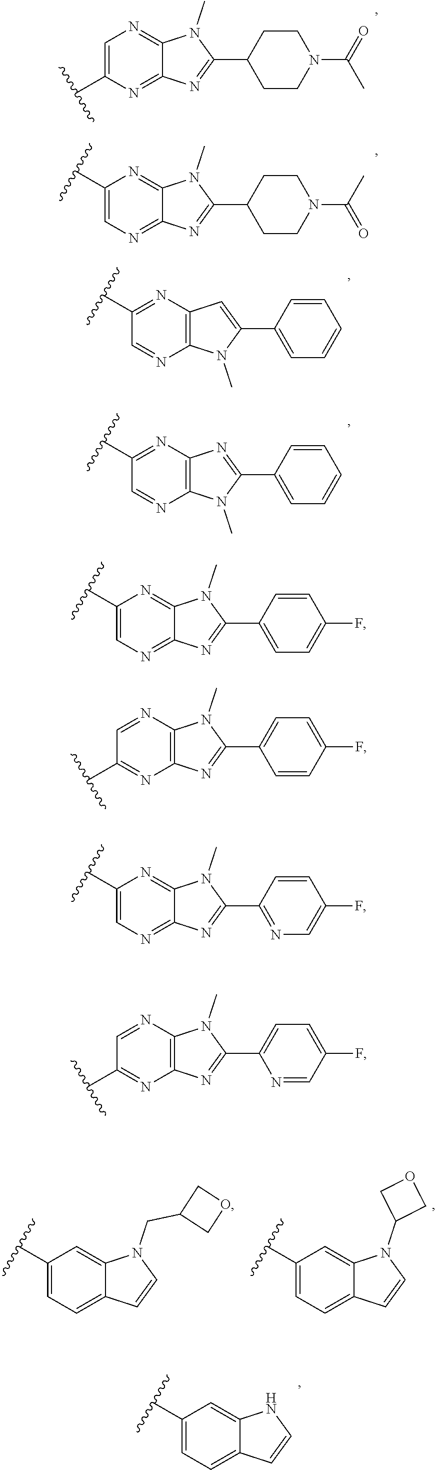

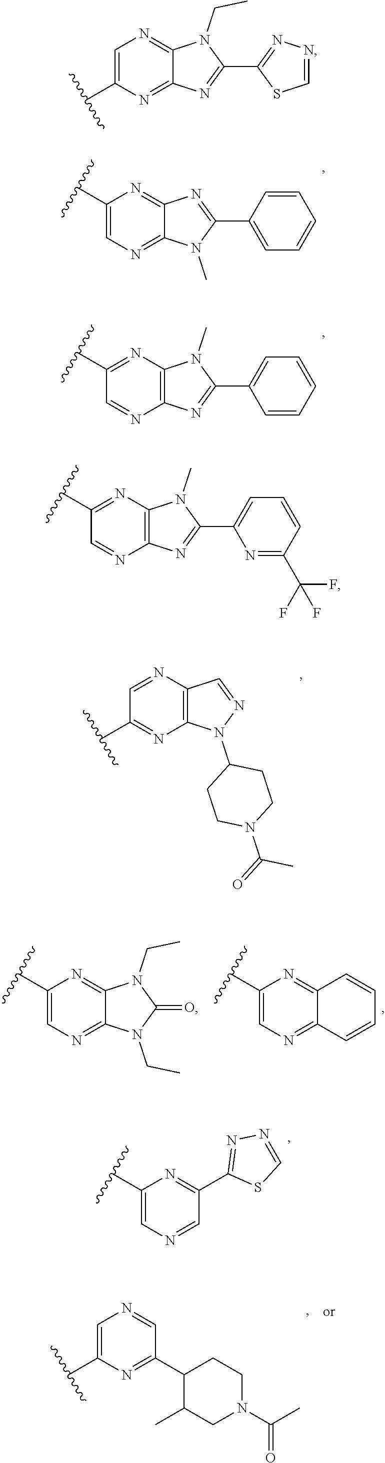

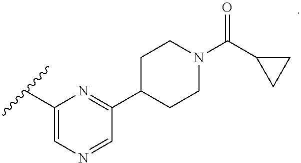 [SMILES: *c1cncc(C2CCN(C(=O)C3CC3)CC2)n1]

Embodiment 64. The compound of any of embodiments 1-53, or a pharmaceutically acceptable salt thereof, wherein: R⁵ is

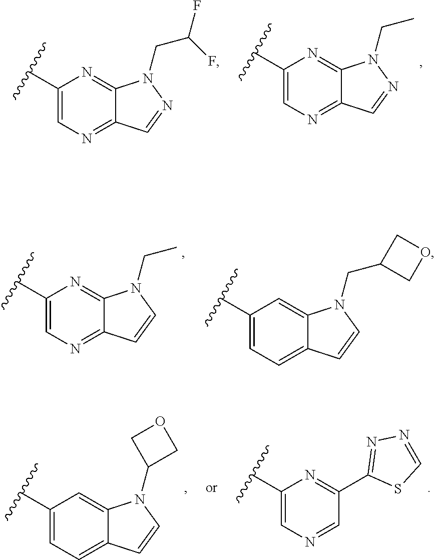

Embodiment 65. The compound of any of embodiments 1-53, or a pharmaceutically acceptable salt thereof, wherein: R⁵ is

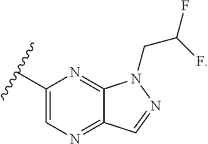 [SMILES: *c1cnc2cnn(CC(F)F)c2n1]

Embodiment 66. The compound of any of embodiments 1-65, or a pharmaceutically acceptable salt thereof, wherein: R² and R³ are each independently hydrogen, or substituted or unsubstituted alkyl.

Embodiment 67. The compound of any of embodiments 1-66, or a pharmaceutically acceptable salt thereof, wherein: R³ is hydrogen.

Embodiment 68. The compound of any of embodiments 1-66, or a pharmaceutically acceptable salt thereof, wherein: R³ is substituted or unsubstituted alkyl.

Embodiment 69. The compound of any of embodiments 1-68, or a pharmaceutically acceptable salt thereof, wherein: R³ is unsubstituted alkyl.

Embodiment 70. The compound of any of embodiments 1-3, wherein the compound is of formula (I-a):

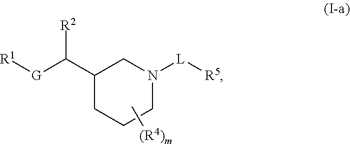

or a pharmaceutically acceptable salt thereof.

Embodiment 71. The compound of any of embodiments 1-3, wherein the compound is of formula (I-b):

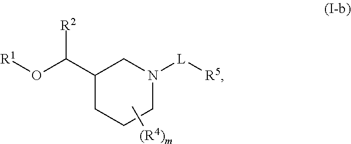

or a pharmaceutically acceptable salt thereof.

Embodiment 72. The compound of any of embodiments 1-3, wherein the compound is of formula (I-c):

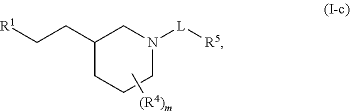

or a pharmaceutically acceptable salt thereof.

Embodiment 73. The compound of any of embodiments 1-3, wherein the compound is of formula (I-d):

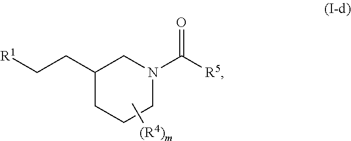

or a pharmaceutically acceptable salt thereof.

Embodiment 74. The compound of any of embodiments 1-3, wherein the compound is of formula (I-e):

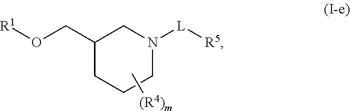

or a pharmaceutically acceptable salt thereof.

Embodiment 75. The compound of any of embodiments 1-3, wherein the compound is of formula (I-f):

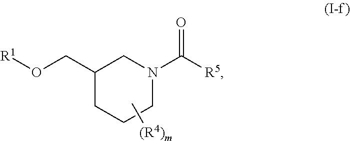

or a pharmaceutically acceptable salt thereof.

Embodiment 76. The compound of any of embodiments 1-3, wherein the compound is of formula (I-g):

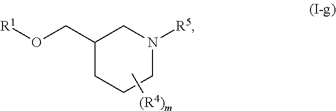

or a pharmaceutically acceptable thereof.

Embodiment 77. The compound of any of embodiments 1-3, wherein the compound is of formula (I-h):

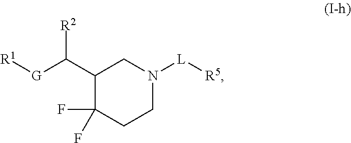

or a pharmaceutically acceptable salt thereof.

Embodiment 78. The compound of any of embodiments 1-3, wherein the compound is of formula (I-i):

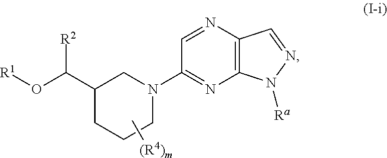

or a pharmaceutically acceptable salt thereof, wherein: R² is hydrogen or alkyl; and R^{a} is substituted or unsubstituted alkyl, or substituted or unsubstituted heterocyclyl.

Embodiment 79. The compound of embodiment 1 or 2, wherein the compound is of formula (II-a):

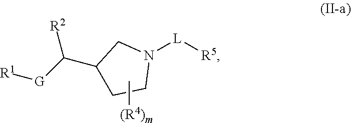

or a pharmaceutically acceptable salt thereof.

Embodiment 80. The compound of embodiment 1 or 2, wherein the compound is of formula (II-b):

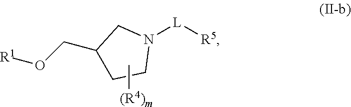

or a pharmaceutically acceptable salt thereof.

Embodiment 81. The compound of embodiment 1 or 2, wherein the compound is of formula (II-c):

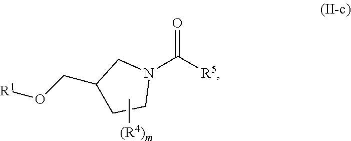

or a pharmaceutically acceptable salt thereof.

Embodiment 82. The compound of embodiment 1 or 2, wherein the compound is of formula (II-d):

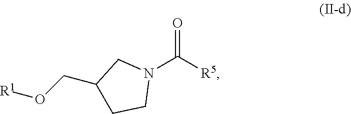

or a pharmaceutically acceptable salt thereof.

Embodiment 83. The compound of embodiment 1 or 2, wherein the compound is of formula (III-a):

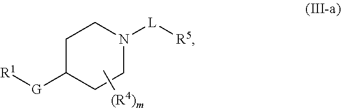

or a pharmaceutically acceptable salt thereof.

Embodiment 84. The compound of embodiment 1 or 2, wherein the compound is of formula (III-b):

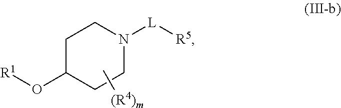

or a pharmaceutically acceptable salt thereof.

Embodiment 85. The compound of embodiment 1 or 2, wherein the compound is of formula (III-c):

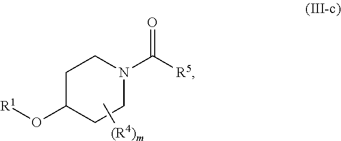

or a pharmaceutically acceptable salt thereof.

Embodiment 86. The compound of embodiment 1 or 2, wherein the compound is of formula (III-d):

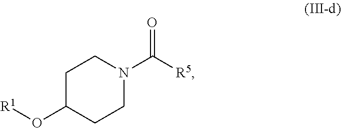

or a pharmaceutically acceptable salt thereof.

Embodiment 87. The compound of any of embodiments 1-3, wherein the compound is of formula (IV-a):

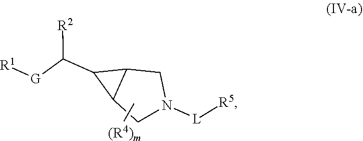

or a pharmaceutically acceptable salt thereof.

Embodiment 88. The compound of any of embodiments 1-3, wherein the compound is of formula (IV-b):

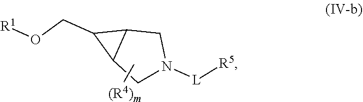

or a pharmaceutically acceptable salt thereof.

Embodiment 89. The compound of any of embodiments 1-3, wherein the compound is of formula (IV-c):

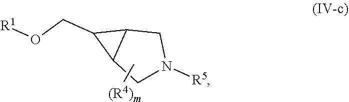

or a pharmaceutically salt thereof.

Embodiment 90. The compound of any of embodiments 1-3, wherein the compound is of formula (IV-d):

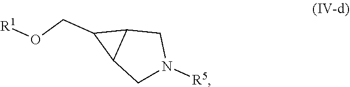

or a pharmaceutically salt thereof.

Embodiment 91. The compound of any of embodiments 1-3, wherein the compound is of formula (IV-e):

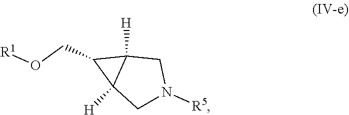

or a pharmaceutically acceptable salt thereof.

Embodiment 92. The compound of embodiment 1 or 2, wherein the compound is of formula (V-a):

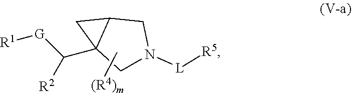

or a pharmaceutically acceptable salt thereof.

Embodiment 93. The compound of embodiment 1 or 2, wherein the compound is of formula (V-b):

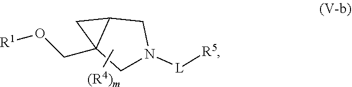

or a pharmaceutically acceptable salt thereof.

Embodiment 94. The compound of embodiment 1 or 2, wherein the compound is of formula (V-c):

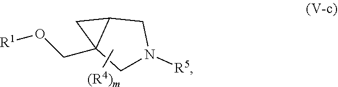

or a pharmaceutically acceptable salt thereof.

Embodiment 95. The compound of embodiment 1 or 2, wherein the compound is of formula (V-d):

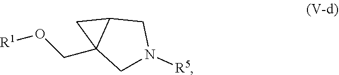

or a pharmaceutically acceptable salt thereof.

Embodiment 96. The compound of any of embodiments 1-3, wherein the compound is a compound of Table 1, or a pharmaceutically acceptable salt thereof.

Embodiment 97. The compound of any of embodiments 1-3, wherein the compound is a compound of Table 2, or a pharmaceutically acceptable salt thereof.

Embodiment 98. The compound of any of embodiments 1-95, wherein the compound is not one or more compound of Table 2, or a pharmaceutically acceptable salt thereof.

Embodiment 99. A pharmaceutical composition comprising a compound of any of embodiments 1-98, or a pharmaceutically acceptable salt thereof, and a pharmaceutically acceptable excipient.

Embodiment 100. A kit comprising a compound of any of embodiments 1-98, or a pharmaceutically acceptable salt thereof, or a pharmaceutical composition of Embodiment 99, and instructions for administering the compound or pharmaceutical composition to a subject in need thereof.

Embodiment 101. A method of treating a disease or disorder in a subject in need thereof, the method comprising administering an effective amount of a compound of any of Embodiments 1-98, or pharmaceutically acceptable salt thereof, or a pharmaceutical composition of embodiment 99.

Embodiment 102. The method of embodiment 101, wherein the disease or disorder is associated with glucocerebrosidase activity.

Embodiment 103. The method of embodiment 101 or 102, wherein the disease or disorder is a neurological disease or disorder.

Embodiment 104. The method of embodiment 103, wherein the neurological disease or disorder is Parkinson's disease or Gaucher's disease.

Embodiment 105. A method of activating glucocerebrosidase, the method comprising contacting glucocerebrosidase with an effective amount of a compound of any of embodiments 1-98, or pharmaceutically acceptable salt thereof, or a pharmaceutical composition of embodiment 99.

Embodiment 106. The method of embodiment 105, wherein the contacting is in vitro.

Embodiment 107. The method of embodiment 105, wherein the contacting is in vivo.

What is claimed is:

1. A compound of Formula (I): 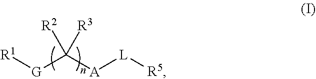
or a pharmaceutically acceptable salt thereof, wherein:
R¹ is substituted or unsubstituted heteroaryl, substituted or unsubstituted aryl, substituted or unsubstituted carbocyclyl, substituted or unsubstituted heterocyclyl, pentyl, butyl, methyl,
—CH₂CH₂CH(CH₃)₂, or hydrogen, or optionally a heterocyclyl forming a spirocyclic ring system with A when n is 0 and G is a bond;
G is a bond, —S(O)₂—, —NR²—, —CH₂CH₂O—, —CH₂O—, —O— or —CR²R³—;
R² and R³ are each independently hydrogen, halogen, or substituted or unsubstituted alkyl, or R² and R³ on the same carbon form with that carbon a carbonyl;
n is 1 or 0;
A is 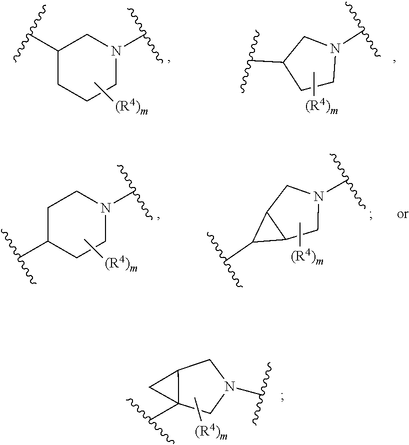
each R⁴ is independently halogen, substituted or unsubstituted alkyl, substituted or unsubstituted alkoxy, hydroxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, —C(═O)CF₂—, —C(═O)CH(Ph)—, —C(═O)CH(iPr)—, —C(═O)CH(Et)—, —C(═O)CH(Me)—, —C(═O)C(CH₃)₂—, —C(═O)CH(OMe)—, —C(═O)CH₂CH₂—, —C(═O)CH₂CH₂CH₂—, —C(═O)CH₂CH₂CH₂O—, —C(═O)CH(CH₃)CH₂—, —C(═O)CH₂O—, —C(═O)CH₂OCH₂—, —C(═O)CH(CH₃)O—, —C(═O)CH₂CH═CH—, —C(═O)NHCH₂CH₂CH₂—, —C(═O)NHCH₂CH₂—, —CH₂—, —CH₂CH₂CH₂—, —CH₂C(CH₃)₂—, —C(═O)NH—, or —CH₂C(═O)NH—; and
R⁵ is substituted or unsubstituted heteroaryl, substituted or unsubstituted heterocyclyl, substituted or unsubstituted heteroarylalkyl, substituted or unsubstituted carbocyclyl, substituted or unsubstituted aryl, methyl, ethyl, butyl, pentyl, t-butyl, —CH₂CH₂CH(CH₃)₂, —SCF₃, or —OCH₂CH(CH₃)₂.

2. A compound of Formula (I): 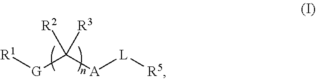
or a pharmaceutically acceptable salt thereof, wherein:
R¹ is substituted or unsubstituted heteroaryl, substituted or unsubstituted aryl, substituted or unsubstituted carbocyclyl, or substituted or unsubstituted heterocyclyl, pentyl, butyl, or
—CH₂CH₂CH(CH₃)₂;
G is —S(O)₂—, —NR²—, —CH₂CH₂O—, —CH₂O—, —O— or —CR²R³—;
R² and R³ are each independently hydrogen, halogen, or substituted or unsubstituted alkyl;
n is 1 or 0;
A is 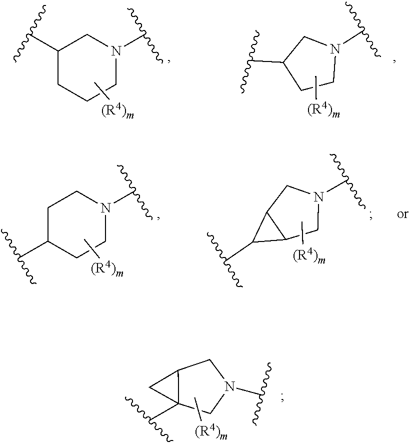
each R⁴ is independently halogen, substituted or unsubstituted alkyl, substituted or unsubstituted alkoxy, or two instances of R⁴ join to form a bridged ring, or two instances of R⁴ on the same carbon form with that carbon a carbonyl;
m is 0, 1, 2, 3, or 4;
L is a bond, —C(═O)—, —C(═O)CH₂—, or —C(═O)CH₂O—; and
R⁵ is substituted or unsubstituted heteroaryl, substituted or unsubstituted heterocyclyl, substituted or unsubstituted heteroarylalkyl, substituted or unsubstituted aryl, substituted or unsubstituted carbocyclyl, or substituted or unsubstituted aryloxyalkyl.

3. A compound of Formula (I): 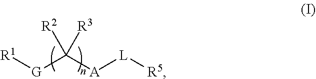
or a pharmaceutically acceptable salt thereof, wherein:
R¹ is substituted or unsubstituted pyridinyl, or substituted or unsubstituted phenyl;
G is —O— or —CR²R³—;
R² and R³ are each independently hydrogen, halogen, or substituted or unsubstituted alkyl;
n is 1 or 0;
A is 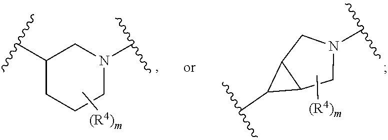
each R⁴ is independently halogen, substituted or unsubstituted alkyl, or two instances of R⁴ on the same carbon form with that carbon a carbonyl;
m is 0, 1, 2, 3, or 4;
L is a bond or —C(═O)—; and
R⁵ is substituted or unsubstituted pyrazolopyrazinyl, substituted or unsubstituted indolyl, substituted or unsubstituted oxadiazolyl, substituted or unsubstituted triazolyl, or substituted or unsubstituted pyrazinyl.

4. The compound of any of claims 1-3, or a pharmaceutically acceptable salt thereof, wherein:
R¹ is substituted or unsubstituted pyrazinyl, substituted or unsubstituted pyridinyl, or substituted or unsubstituted phenyl.

5. The compound of any of claims 1-3, or a pharmaceutically acceptable salt thereof, wherein:
R¹ is substituted pyridinyl, or substituted or unsubstituted phenyl.

6. The compound of any of claims 1-3, or a pharmaceutically acceptable salt thereof, wherein:
R¹ is pyridinyl substituted with haloalkyl or haloalkoxy, unsubstituted phenyl, or phenyl substituted with halogen, haloalkyl, or alkyl.

7. The compound of any of claims 1-4, or a pharmaceutically acceptable salt thereof, wherein:
R¹ is pyridinyl substituted with haloalkyl or haloalkoxy.

8. The compound of any of claims 1-7, or a pharmaceutically acceptable salt thereof, wherein:
R¹ is pyridinyl substituted with haloalkyl.

9. The compound of any of claims 1-6, or a pharmaceutically acceptable salt thereof, wherein:
R¹ is unsubstituted phenyl.

10. The compound of any of claims 1-6, or a pharmaceutically acceptable salt thereof, wherein:
R¹ is phenyl substituted with halogen, haloalkyl, or alkyl.

11. The compound of any of claims 1-6, or a pharmaceutically acceptable salt thereof, wherein:
R¹ is phenyl substituted with haloalkyl.

12. The compound of any of claims 1-3, or a pharmaceutically acceptable salt thereof, wherein:
R¹ is hydrogen, methyl, butyl, pentyl, —CH₂CH₂CH(CH₃)₂,

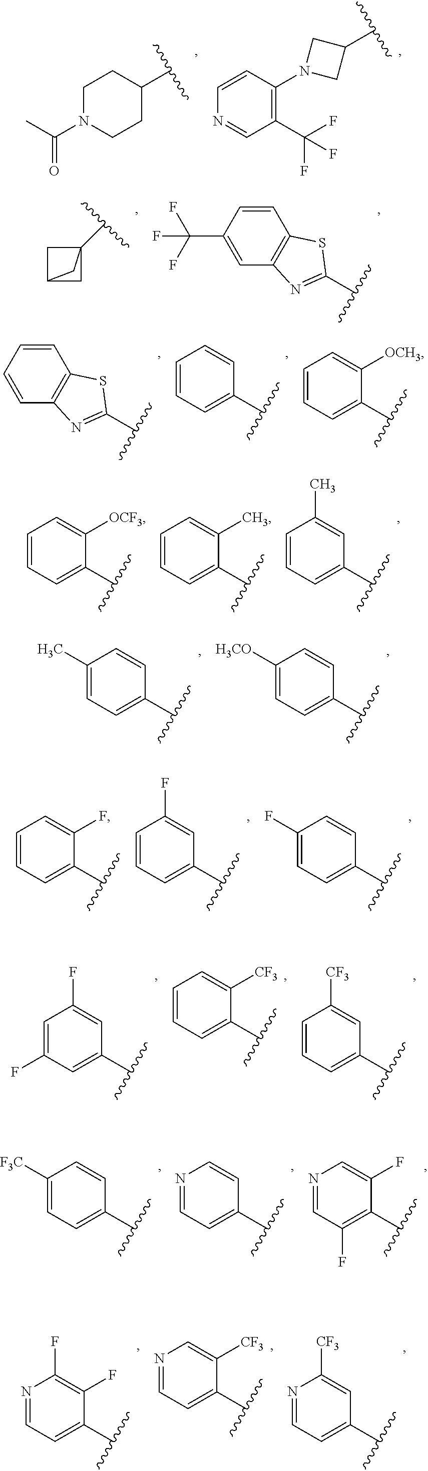

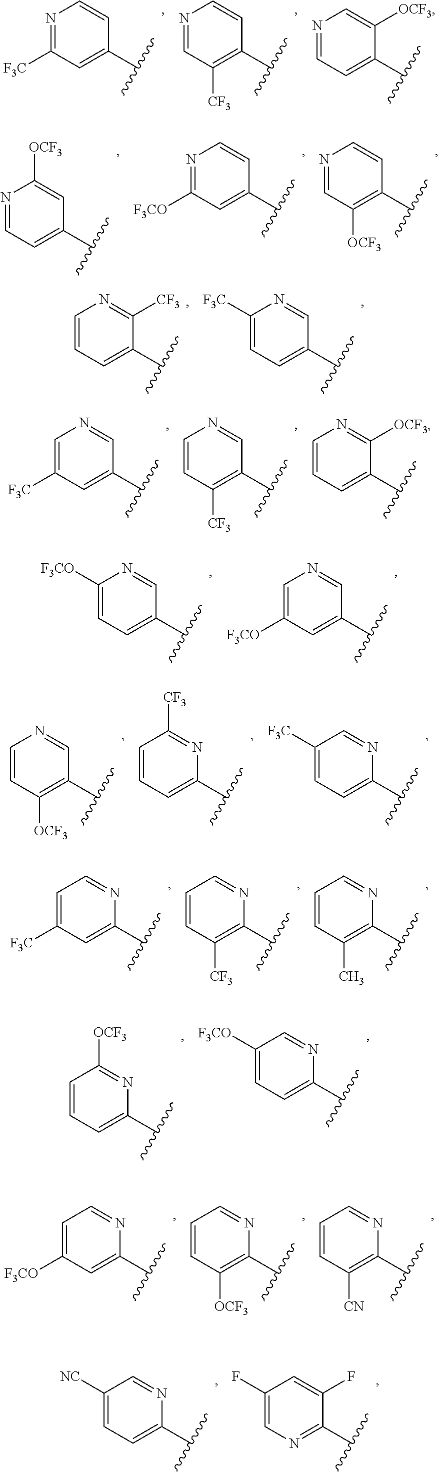

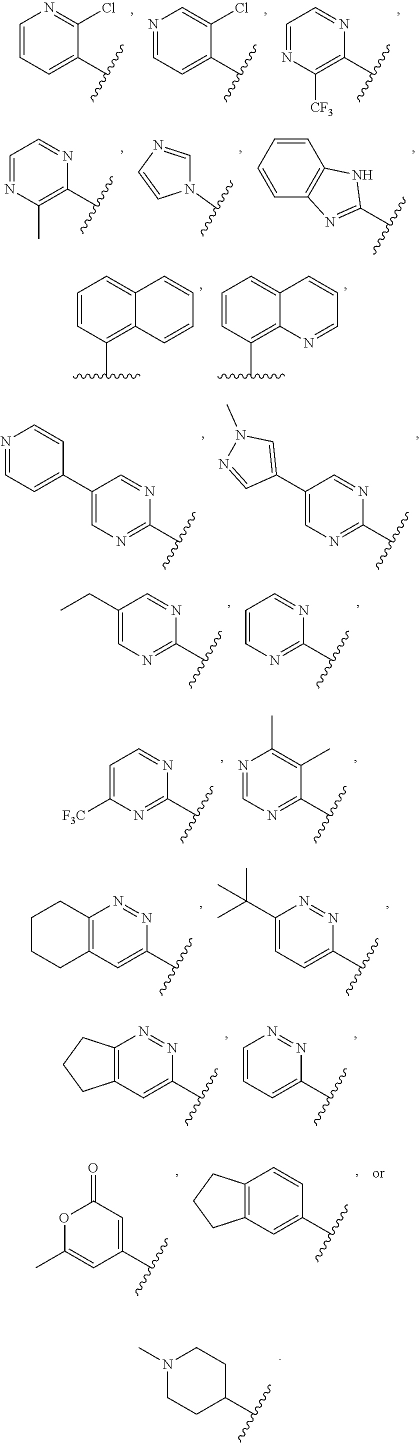

13. The compound of any of claims 1-3, or a pharmaceutically acceptable salt thereof, wherein:
R¹ is butyl, pentyl, —CH₂CH₂CH(CH₃)₂,

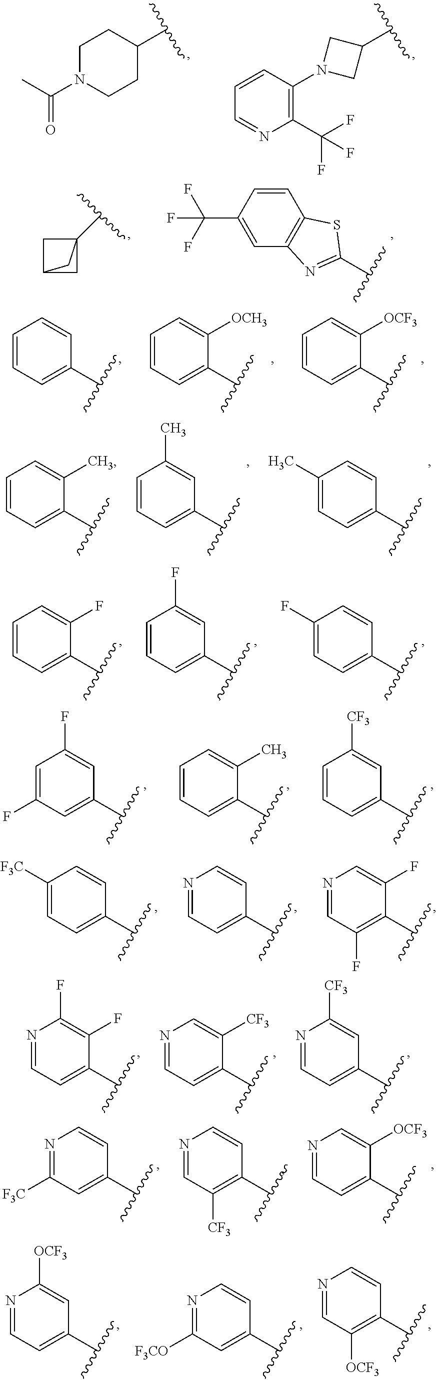

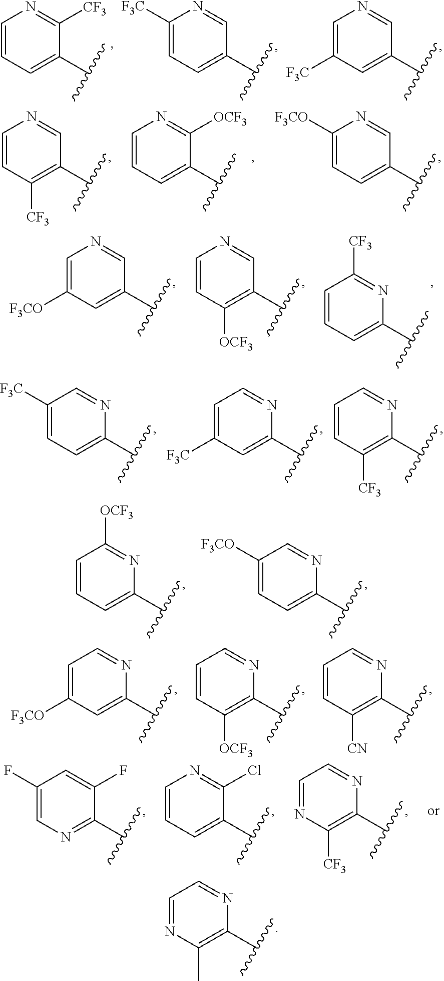

14. The compound of any of claims 1-6, or a pharmaceutically acceptable salt thereof, wherein:
R¹ is

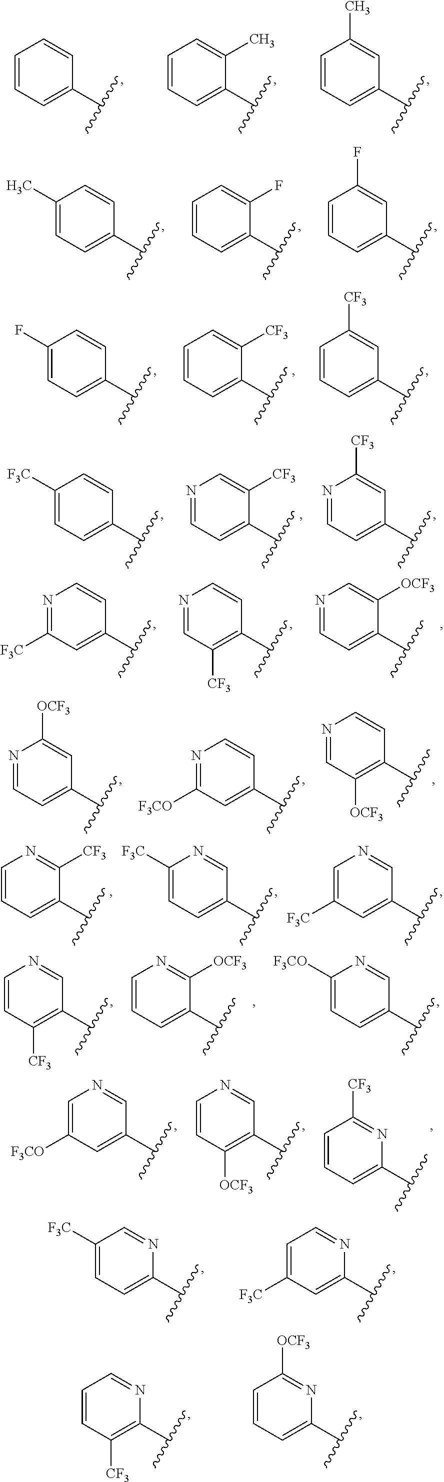

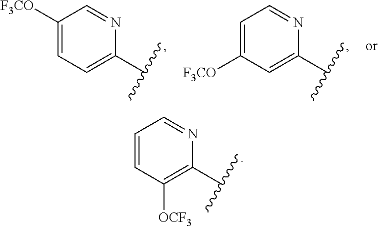 [SMILES: *c1ccc(OC(F)(F)F)cn1 ; *c1cc(OC(F)(F)F)ccn1 ; *c1ncccc1OC(F)(F)F]

15. The compound of any of claims 1-6, or a pharmaceutically acceptable salt thereof, wherein:
R¹ is

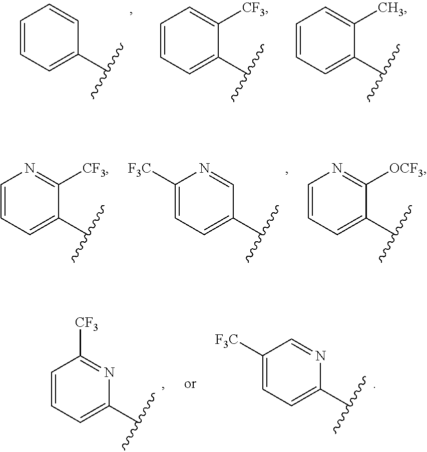

16. The compound of any of claims 1-6, or a pharmaceutically acceptable salt thereof, wherein:
R¹ is

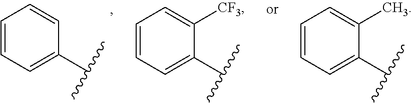 [SMILES: *c1ccccc1 ; *c1ccccc1C(F)(F)F ; *c1ccccc1C]

17. The compound of any of claims 1-6, or a pharmaceutically acceptable salt thereof, wherein:
R¹ is

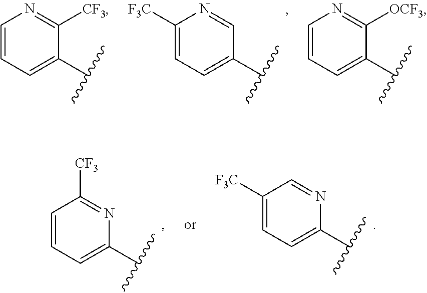 [SMILES: *c1cccnc1C(F)(F)F ; *c1ccc(C(F)(F)F)nc1 ; *c1cccnc1OC(F)(F)F ; *c1cccc(C(F)(F)F)n1 ; *c1ccc(C(F)(F)F)cn1]

18. The compound of any of claims 1-17, or a pharmaceutically acceptable salt thereof, wherein:
G is —O—.

19. The compound of any of claims 1-17, or a pharmaceutically acceptable salt thereof, wherein:
G is —CR²R³—.

20. The compound of any of claims 1-17, or a pharmaceutically acceptable salt thereof, wherein:
G is —CH₂— or —CH(CH₃)—.

21. The compound of any of claims 1-17, or a pharmaceutically acceptable salt thereof, wherein:
G is —CH₂—.

22. The compound of any of claims 1-17, or a pharmaceutically acceptable salt thereof, wherein:
G is —CH(CH₃)—.

23. The compound of any of claims 1-22, or a pharmaceutically acceptable salt thereof, wherein:
n is 1.

24. The compound of any of claims 1-22, or a pharmaceutically acceptable salt thereof, wherein:
n is 0.

25. The compound of any of claims 1-24, or a pharmaceutically acceptable salt thereof, wherein:
when n is 0, then A is

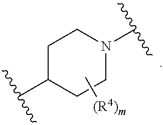

26. The compound of any of claims 1-24, or a pharmaceutically acceptable salt thereof, wherein:
A is

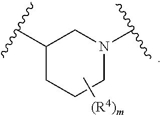

27. The compound of any of claims 1-24, or a pharmaceutically acceptable salt thereof, wherein:
A is

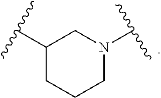 [SMILES: *C1CCCN(*)C1]

28. The compound of any of claims 1-24, or a pharmaceutically acceptable salt thereof, wherein:
A is

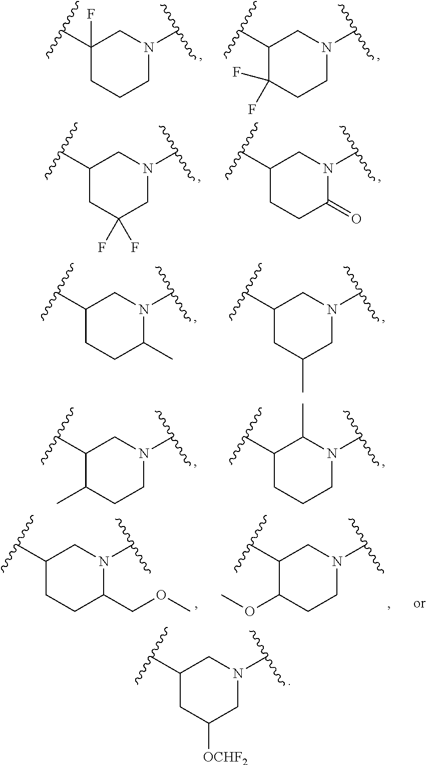

29. The compound of any of claims 1-24, or a pharmaceutically acceptable salt thereof, wherein:
A is

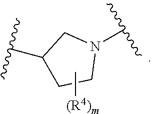

30. The compound of any of claims 1-24, or a pharmaceutically acceptable salt thereof, wherein:
A is

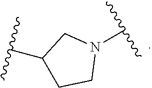 [SMILES: *C1CCN(*)C1]

31. The compound of any of claims 1-24, or a pharmaceutically acceptable salt thereof, wherein:
A is

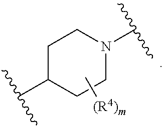

32. The compound of any of claims 1-24, or a pharmaceutically acceptable salt thereof, wherein:
A is

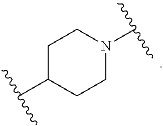 [SMILES: *C1CCN(*)CC1]

33. The compound of any of claims 1-24, or a pharmaceutically acceptable salt thereof, wherein:
A is

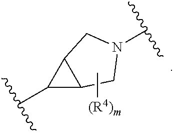

34. The compound of any of claims 1-24, or a pharmaceutically acceptable salt thereof, wherein:
A is

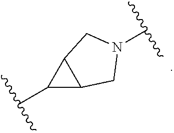 [SMILES: *C1C2CN(*)CC12]

35. The compound of any of claims 1-24, or a pharmaceutically acceptable salt thereof, wherein:
A is

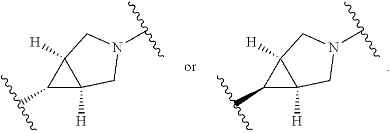 [SMILES: *[C@@H]1[C@H]2CN(*)C[C@@H]12 ; *[C@H]1[C@H]2CN(*)C[C@@H]12]

36. The compound of any of claims 1-24, or a pharmaceutically acceptable salt thereof, wherein:
A is

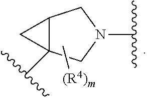

37. The compound of any of claims 1-24, or a pharmaceutically acceptable salt thereof, wherein:
A is

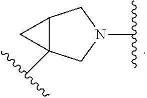 [SMILES: *N1CC2CC2(*)C1]

38. The compound of any of claims 1-37, or a pharmaceutically acceptable salt thereof, wherein:
each R⁴ is independently halogen, or two instances of R⁴ on the same carbon form with that carbon a carbonyl.

39. The compound of any of claims 1-38, or a pharmaceutically acceptable salt thereof, wherein:
each R⁴ is independently R⁴ is fluoro, or two instances of R⁴ on the same carbon form with that carbon a carbonyl.

40. The compound of any of claims 1-39, or a pharmaceutically acceptable salt thereof, wherein:
R⁴ is fluoro.

41. The compound of any of claims 1-37, or a pharmaceutically acceptable salt thereof, wherein:
each R⁴ is independently fluoro, methyl, CH₃OCH₂—, methoxy, difluoromethoxy, or two instances of R⁴ on the same carbon form with that carbon a carbonyl.

42. The compound of any of claims 1-41, or a pharmaceutically acceptable salt thereof, wherein:
m is 0.

43. The compound of any of claims 1-41, or a pharmaceutically acceptable salt thereof, wherein:
m is 2.

44. The compound of any of claims 1-43, or a pharmaceutically acceptable salt thereof, wherein:
L is a bond.

45. The compound of any of claims 1-43, or a pharmaceutically acceptable salt thereof, wherein:
L is —C(═O)—.

46. The compound of any of claims 1-43, or a pharmaceutically acceptable salt thereof, wherein:
L is —C(═O)CH₂—.

47. The compound of any of claims 1-43 or a pharmaceutically acceptable salt thereof, wherein:
L is —C(═O)CH₂O—.

48. The compound of any of claims 1-47, or a pharmaceutically acceptable salt thereof, wherein:
R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted imidazopyridinyl, substituted or unsubstituted triazolopyridinyl, substituted or unsubstituted pyrazolopyrimidinyl, substituted or unsubstituted pyrrolopyrimidinyl, substituted or unsubstituted chromenonyl, substituted or unsubstituted isochromanyl, substituted or unsubstituted indolyl, substituted or unsubstituted quinoxalinyl, substituted or unsubstituted benzofuranyl, substituted or unsubstituted benzothiophenyl, substituted or unsubstituted benzimidazolyl, substituted or unsubstituted benzoxazolyl, substituted or unsubstituted 5,6,7,8-tetrahydro-[1.2.4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1.4]dioxinyl, substituted or unsubstituted tetrahydronaphthalenyl, substituted or unsubstituted isoquinolinonyl, substituted or unsubstituted quinolinyl, substituted or unsubstituted naphthyridinyl, substituted or unsubstituted naphthyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted imidazolyl, substituted or unsubstituted pyrrolyl, substituted or unsubstituted thiophenyl, substituted or unsubstituted furanyl, substituted or unsubstituted isothiazolyl, substituted or unsubstituted isoxazolyl, substituted or unsubstituted isoxazolonyl, substituted or unsubstituted 3,4-dihydro-1H-pyrrolo[2,1-c][1.4]thiazin-8-yl, substituted or unsubstituted pyrrolidinonyl, substituted or unsubstituted pyrrolidinyl, substituted or unsubstituted 1,4-diazepanyl, substituted or unsubstituted dioxolanonyl, substituted or unsubstituted pyridinyl, substituted or unsubstituted pyrazinyl, substituted or unsubstituted pyridazinyl, substituted or unsubstituted pyrimidinyl, substituted or unsubstituted phenyl, substituted or unsubstituted tetrahydropyranyl, substituted or unsubstituted tetrahydrofuranyl, substituted or unsubstituted morpholinyl, substituted or unsubstituted cyclooctyl, substituted or unsubstituted cyclohexyl, substituted or unsubstituted cyclopentyl, substituted or unsubstituted cyclobutyl, substituted or unsubstituted cyclopropyl, substituted or unsubstituted bicyclo[3.3.1]nonanyl, substituted or unsubstituted bicyclo[2.2.1]heptanyl, substituted or unsubstituted 7-oxaspiro[3.5]non-1-en-2-yl, substituted or unsubstituted hexahydro-1H-cyclopenta[c]furan-5-yl, substituted or unsubstituted adamantyl, substituted or unsubstituted spiro[2.5]octan-4-yl, methyl, ethyl, butyl, pentyl, t-butyl,
—CH₂CH₂CH(CH₃)₂, —SCF₃, or —OCH₂CH(CH₃)₂.

49. The compound of any of claims 1-47, or a pharmaceutically acceptable salt thereof, wherein:
R⁵ is substituted or unsubstituted pyrazolopyrazinyl, substituted or unsubstituted pyrrolopyrazinyl, substituted or unsubstituted imidazopyrazinyl, substituted or unsubstituted pyrazolopyridinyl, substituted or unsubstituted pyrrolopyridinyl, substituted or unsubstituted pyrazolopyrimidinyl, substituted or unsubstituted indolyl, substituted or unsubstituted quinoxalinyl, substituted or unsubstituted benzofuranyl, substituted or unsubstituted 5,6,7,8-tetrahydro-[1,2,4]triazolo[4,3-a]pyrazinyl, substituted or unsubstituted pyrrolo[3,2-c]pyridin-4-onyl, substituted or unsubstituted 7,8-dihydropyrrolo[1,2-a]pyrimidin-4(6H)-onyl, substituted or unsubstituted 1,5-dihydro-4H-pyrazolo[4,3-c]pyridin-4-onyl, substituted or unsubstituted 2,3-dihydrobenzo[b][1,4]dioxinyl, substituted or unsubstituted tetrahydronaphthalenyl, substituted or unsubstituted pyridazinonyl, substituted or unsubstituted pyridinonyl, substituted or unsubstituted oxadiazolyl, substituted or unsubstituted thiadiazolyl, substituted or unsubstituted thiazolyl, substituted or unsubstituted pyrazolyl, substituted or unsubstituted triazolyl, substituted or unsubstituted pyrazinyl, substituted or unsubstituted pyrimidinyl, substituted or unsubstituted phenyl, substituted or unsubstituted tetrahydropyranyl, substituted or unsubstituted morpholinyl, or substituted or unsubstituted cyclopentyl.

50. The compound of any of claims 1-49, or a pharmaceutically acceptable salt thereof, wherein:
R⁵ is substituted or unsubstituted pyrazolopyrazinyl, substituted or unsubstituted pyrrolopyrazinyl, substituted or unsubstituted indolyl, substituted or unsubstituted oxadiazolyl, substituted or unsubstituted pyrazolyl, substituted or unsubstituted triazolyl, substituted or unsubstituted pyrazinyl, substituted or unsubstituted tetrahydropyranyl, substituted or unsubstituted pyrazolylmethyl, substituted or unsubstituted indolylmethyl, substituted or unsubstituted cyclohexyl, or substituted or unsubstituted phenyloxyalkyl.

51. The compound of any of claims 1-50, or a pharmaceutically acceptable salt thereof, wherein:
R⁵ is substituted pyrazolopyrazinyl, substituted pyrrolopyrazinyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl.

52. The compound of any of claims 1-51, or a pharmaceutically acceptable salt thereof, wherein:
R⁵ is substituted pyrazolopyrazinyl, substituted pyrrolopyrazinyl, substituted indolyl, substituted oxadiazolyl, substituted pyrazolyl, substituted triazolyl, substituted pyrazinyl, substituted tetrahydropyranyl, substituted pyrazolylmethyl, unsubstituted indolylmethyl, substituted cyclohexyl, or substituted phenyloxypropyl, wherein each substituted R⁵ is substituted with haloalkyl, cycloalkyl, heteroaryl, aryl, halogen, arylalkyl, alkoxy, alkyl, heterocyclylalkyl, or heterocyclyl.

53. The compound of any of claims 1-47, or a pharmaceutically acceptable salt thereof, wherein:
R⁵ is 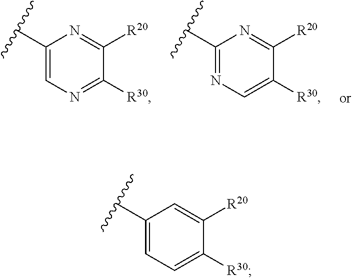
R²⁰ and R³⁰ are each independently hydrogen, halogen, substituted or unsubstituted alkyl, substituted or unsubstituted carbocyclyl, substituted or unsubstituted heterocyclyl, substituted or unsubstituted aryl, or substituted or unsubstituted heteroaryl; or R²⁰ and R³⁰ together with the atoms to which they are attached form a substituted or unsubstituted carbocyclyl, substituted or unsubstituted heterocyclyl, substituted or unsubstituted aryl, or substituted or unsubstituted heteroaryl.

54. The compound of claim 53, or a pharmaceutically acceptable salt thereof, wherein:
A is

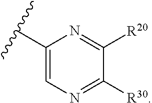

55. The compound of claim 53 or 54, or a pharmaceutically acceptable salt thereof, wherein:
R²⁰ and R³⁰ are each independently hydrogen or substituted or unsubstituted heteroaryl; or R²⁰ and R³⁰ together with the atoms to which they are attached form a substituted or unsubstituted aryl, or substituted or unsubstituted heteroaryl.

56. The compound of any of claims 53-55, or a pharmaceutically acceptable salt thereof, wherein:
R²⁰ is heteroaryl.

57. The compound of any of claims 53-56, or a pharmaceutically acceptable salt thereof, wherein:
R²⁰ is thiadiazolyl.

58. The compound of any of claims 53-57, or a pharmaceutically acceptable salt thereof, wherein:
R³⁰ is hydrogen.

59. The compound of any of claims 53-55, or a pharmaceutically acceptable salt thereof, wherein:
R²⁰ and R³⁰ together with the atoms to which they are attached form a substituted or unsubstituted aryl, or substituted or unsubstituted heteroaryl.

60. The compound of any of claims 53-55, or a pharmaceutically acceptable salt thereof, wherein:
R²⁰ and R³⁰ together with the atoms to which they are attached form a substituted or unsubstituted heteroaryl.

61. The compound of any of claims 53-55, or a pharmaceutically acceptable salt thereof, wherein:
R²⁰ and R³⁰ together with the atoms to which they are attached form a substituted or unsubstituted imidazolyl, substituted or unsubstituted pyrrolyl, or a substituted or unsubstituted pyrazolyl.

62. The compound of any of claims 53-55, or a pharmaceutically acceptable salt thereof, wherein:
R²⁰ and R³⁰ together with the atoms to which they are attached form a substituted or unsubstituted pyrazolyl.

63. The compound of any of claims 1-53, or a pharmaceutically acceptable salt thereof, wherein:
R⁵ is

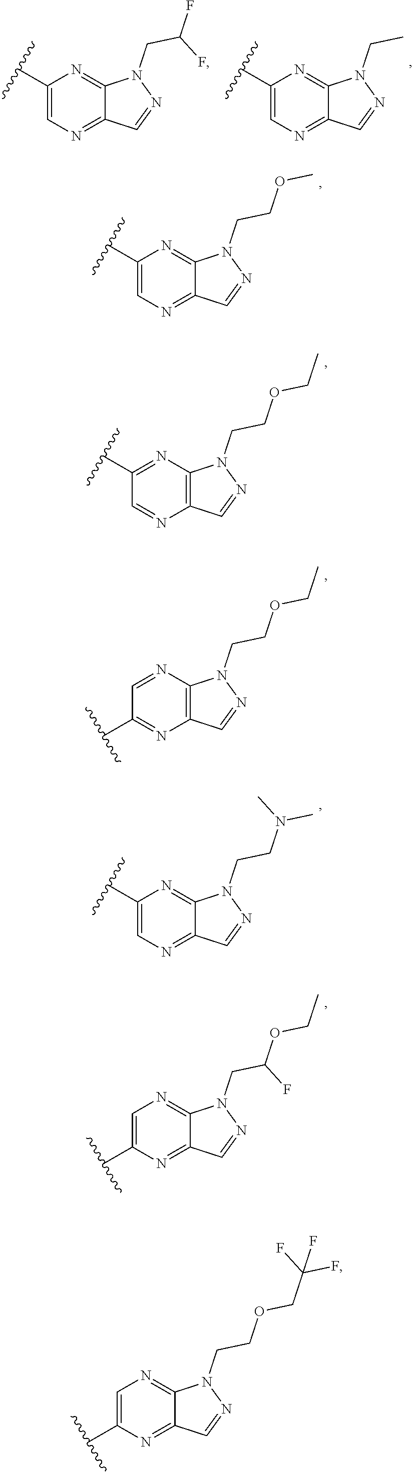

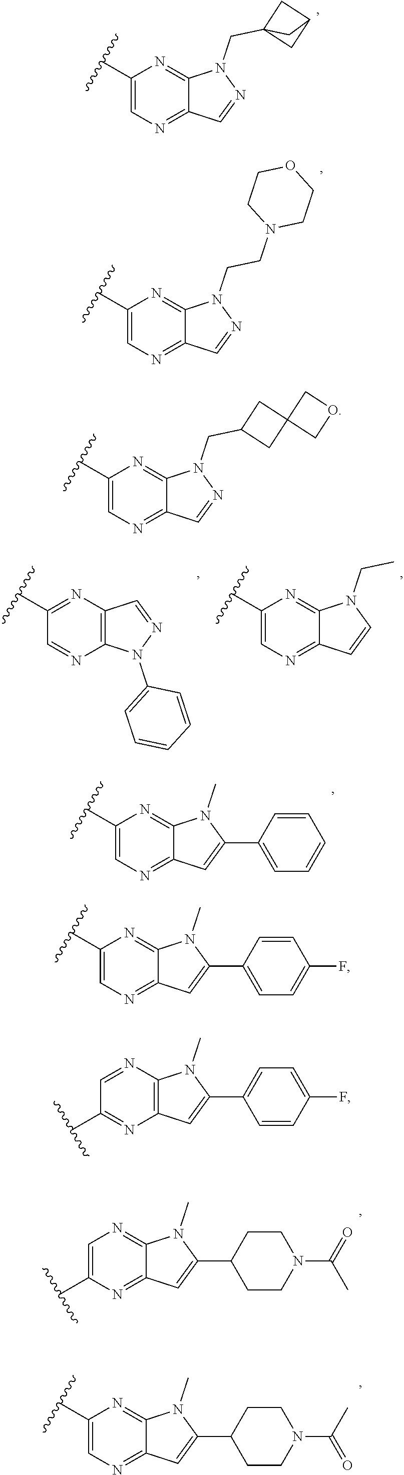

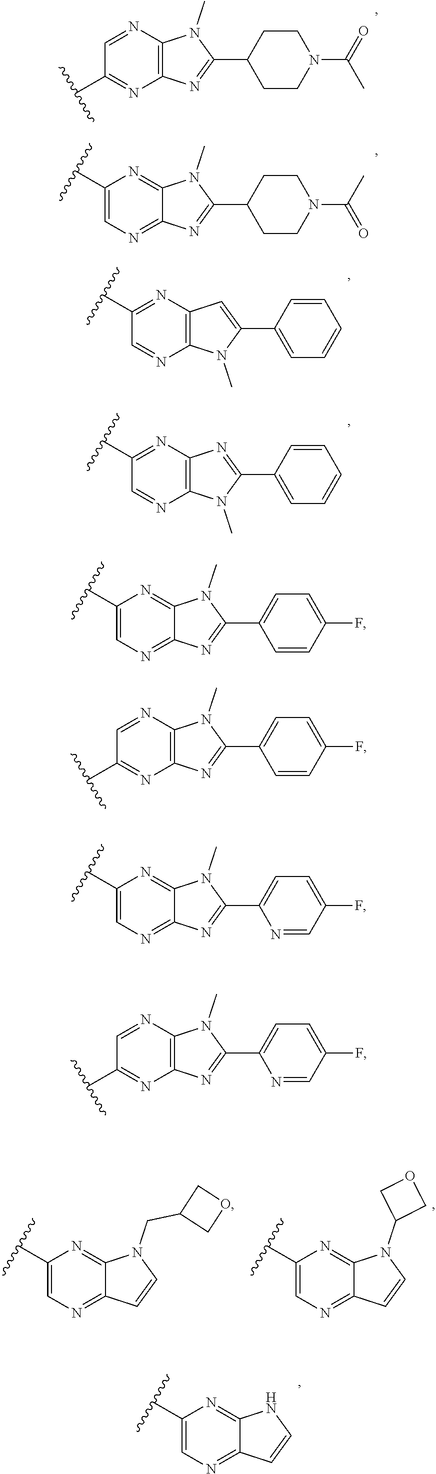

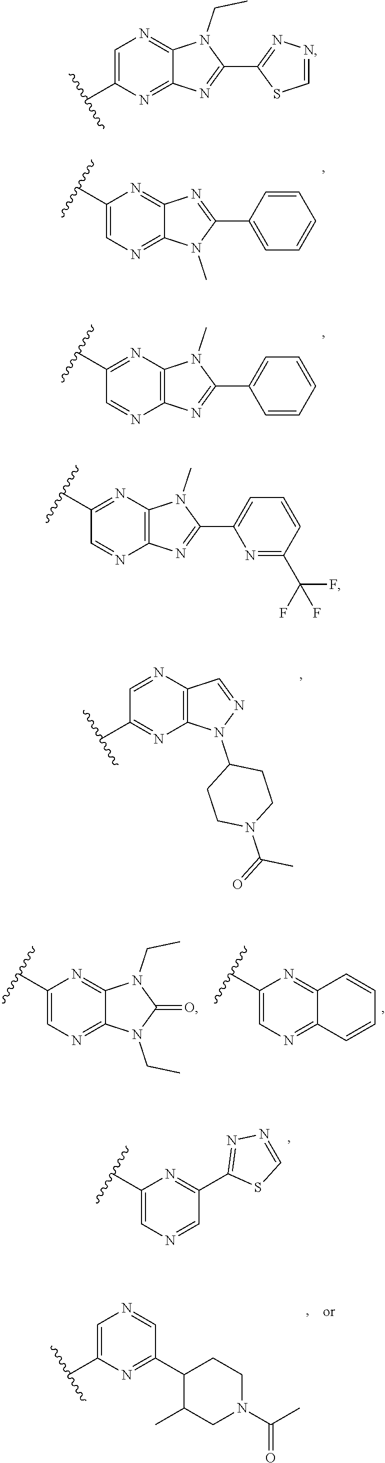

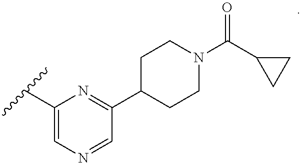 [SMILES: *c1cncc(C2CCN(C(=O)C3CC3)CC2)n1]

64. The compound of any of claims 1-53, or a pharmaceutically acceptable salt thereof, wherein:
R⁵ is

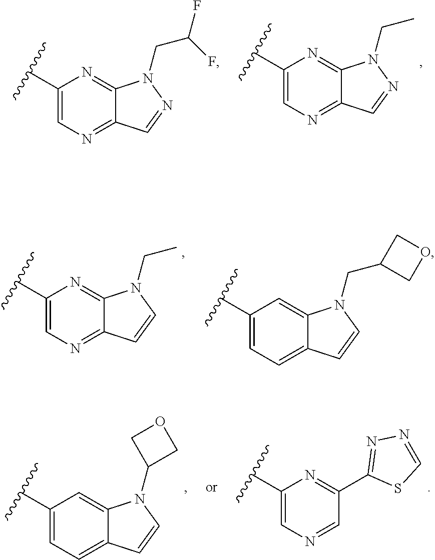

65. The compound of any of claims 1-53, or a pharmaceutically acceptable salt thereof, wherein:
R⁵ is

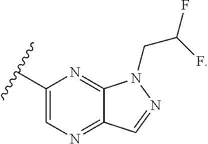 [SMILES: *c1cnc2cnn(CC(F)F)c2n1]

66. The compound of any of claims 1-65, or a pharmaceutically acceptable salt thereof, wherein:
R² and R³ are each independently hydrogen, or substituted or unsubstituted alkyl.

67. The compound of any of claims 1-66, or a pharmaceutically acceptable salt thereof, wherein:
R³ is hydrogen.

68. The compound of any of claims 1-66, or a pharmaceutically acceptable salt thereof, wherein:
R³ is substituted or unsubstituted alkyl.

69. The compound of any of claims 1-68, or a pharmaceutically acceptable salt thereof, wherein:
R³ is unsubstituted alkyl.

70. The compound of any of claims 1-3, wherein the compound is of formula (I-a): 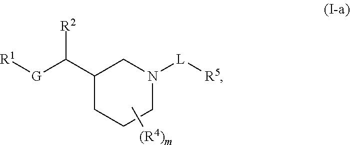
or a pharmaceutically acceptable salt thereof.

71. The compound of any of claims 1-3, wherein the compound is of formula (I-b): 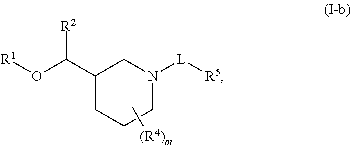
or a pharmaceutically acceptable salt thereof.

72. The compound of any of claims 1-3, wherein the compound is of formula (I-c): 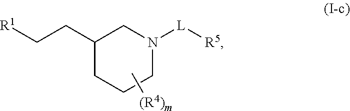
or a pharmaceutically acceptable salt thereof.

73. The compound of any of claims 1-3, wherein the compound is of formula (I-d): 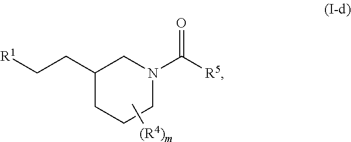
or a pharmaceutically acceptable salt thereof.

74. The compound of any of claims 1-3, wherein the compound is of formula (I-e): 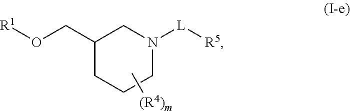
or a pharmaceutically acceptable salt thereof.

75. The compound of any of claims 1-3, wherein the compound is of formula (I-f): 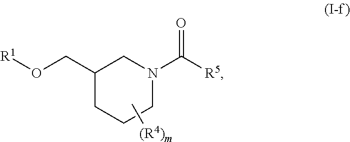
or a pharmaceutically acceptable salt thereof.

76. The compound of any of claims 1-3, wherein the compound is of formula (I-g): 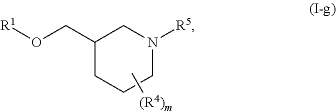
or a pharmaceutically acceptable salt thereof.

77. The compound of any of claims 1-3, wherein the compound is of formula (I-h): 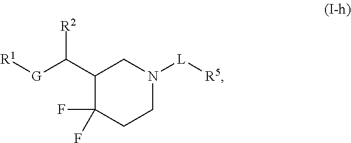
or a pharmaceutically acceptable salt thereof.

78. The compound of any of claims 1-3, wherein the compound is of formula (I-i): 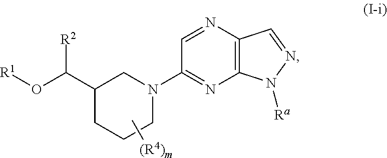
or a pharmaceutically acceptable salt thereof, wherein:
R² is hydrogen or alkyl; and
R^{a} is substituted or unsubstituted alkyl, or substituted or unsubstituted heterocyclyl.

79. The compound of claim 1 or 2, wherein the compound is of formula (II-a): 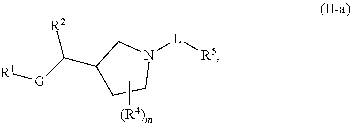
or a pharmaceutically acceptable salt thereof.

80. The compound of claim 1 or 2, wherein the compound is of formula (II-b): 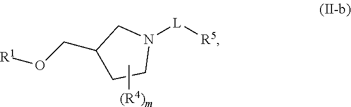
or a pharmaceutically acceptable salt thereof.

81. The compound of claim 1 or 2, wherein the compound is of formula (II-c): 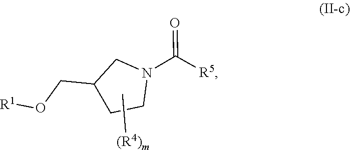
or a pharmaceutically acceptable salt thereof.

82. The compound of claim 1 or 2, wherein the compound is of formula (II-d): 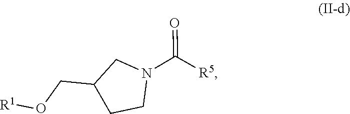
or a pharmaceutically acceptable salt thereof.

83. The compound of claim 1 or 2, wherein the compound is of formula (III-a): 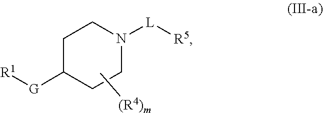
or a pharmaceutically acceptable salt thereof.

84. The compound of claim 1 or 2, wherein the compound is of formula (III-b): 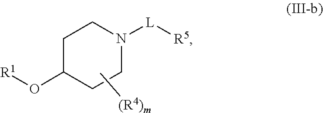
or a pharmaceutically acceptable salt thereof.

85. The compound of claim 1 or 2, wherein the compound is of formula (III-c): 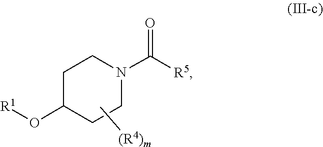
or a pharmaceutically acceptable salt thereof.

86. The compound of claim 1 or 2, wherein the compound is of formula (III-d): 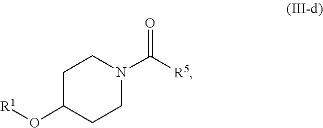
or a pharmaceutically acceptable salt thereof.

87. The compound of any of claims 1-3, wherein the compound is of formula (IV-a): 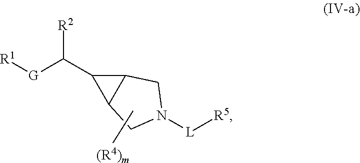
or a pharmaceutically acceptable salt thereof.

88. The compound of any of claims 1-3, wherein the compound is of formula (IV-b): 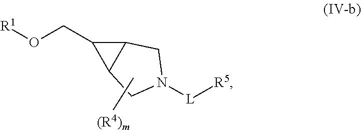
or a pharmaceutically acceptable salt thereof.

89. The compound of any of claims 1-3, wherein the compound is of formula (IV-c): 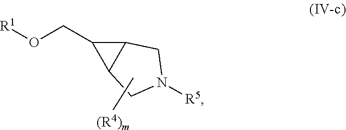
or a pharmaceutically acceptable salt thereof.

90. The compound of any of claims 1-3, wherein the compound is of formula (IV-d): 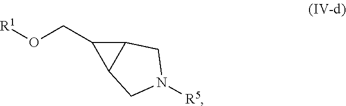
or a pharmaceutically acceptable salt thereof.

91. The compound of any of claims 1-3, wherein the compound is of formula (IV-e): 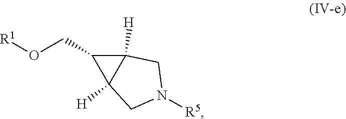
or a pharmaceutically acceptable salt thereof.

92. The compound of claim 1 or 2, wherein the compound is of formula (V-a): 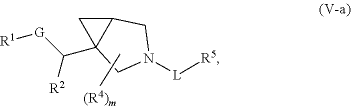
or a pharmaceutically acceptable salt thereof.

93. The compound of claim 1 or 2, wherein the compound is of formula (V-b): 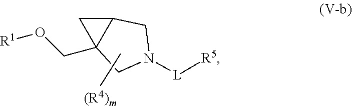
or a pharmaceutically acceptable salt thereof.

94. The compound of claim 1 or 2, wherein the compound is of formula (V-c): 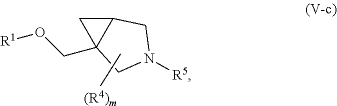
or a pharmaceutically acceptable salt thereof.

95. The compound of claim 1 or 2, wherein the compound is of formula (V-d): 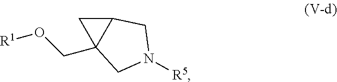
or a pharmaceutically acceptable salt thereof.

96. The compound of any of claims 1-3, wherein the compound is a compound of Table 1, or a pharmaceutically acceptable salt thereof.

97. The compound of any of claims 1-3, wherein the compound is a compound of Table 2, or a pharmaceutically acceptable salt thereof.

98. The compound of any of claims 1-95, wherein the compound is not one or more compound of Table 2, or a pharmaceutically acceptable salt thereof.

99. A pharmaceutical composition comprising a compound of any of claims 1-98, or a pharmaceutically acceptable salt thereof, and a pharmaceutically acceptable excipient.

100. A kit comprising a compound of any of claims 1-98, or a pharmaceutically acceptable salt thereof, or a pharmaceutical composition of claim 99, and instructions for administering the compound or pharmaceutical composition to a subject in need thereof.

101. A method of treating a disease or disorder in a subject in need thereof, the method comprising administering an effective amount of a compound of any of claims 1-98, or pharmaceutically acceptable salt thereof, or a pharmaceutical composition of claim 99.

102. The method of claim 101, wherein the disease or disorder is associated with glucocerebrosidase activity.

103. The method of claim 101 or 102, wherein the disease or disorder is a neurological disease or disorder.

104. The method of claim 103, wherein the neurological disease or disorder is Parkinson's disease or Gaucher's disease.

105. A method of activating glucocerebrosidase, the method comprising contacting glucocerebrosidase with an effective amount of a compound of any of claims 1-98, or pharmaceutically acceptable salt thereof, or a pharmaceutical composition of claim 99.

106. The method of claim 105, wherein the contacting is in vitro.

107. The method of claim 105, wherein the contacting is in vivo.
